(19) United States
(12) Patent Application Publication
(10) Pub. No.: US 2026/0221227 A1
(43) Pub. Date: Jul. 30, 2026

(54) SYSTEMS AND METHODS FOR MOLECULAR ENDOTYPING OF RHEUMATOID ARTHRITIS

(71) Applicant: AMPEL BioSolutions, LLC, Charlottesville, VA (US)

(72) Inventors: Peter E. LIPSKY, Charlottesville, VA (US); Prathyusha BACHALI, Charlottesville, VA (US); Robert ROBL, Charlottesville, VA (US); Amrie C. GRAMMER, Charlottesville, VA (US)

(21) Appl. No.: 19/407,806

(22) Filed: Dec. 3, 2025

Related U.S. Application Data
(60) Provisional application No. 63/727,799, filed on Dec. 4, 2024.

(51) Int. Cl. G16B 25/10; A61K 45/00; C12Q 1/686; G16B 40/20; G16H 20/10; G16H 50/20

(52) U.S. Cl. CPC G16B 25/10; A61K 45/05; C12Q 1/686; G16B 40/20; G16H 20/10; G16H 50/20; C12Q 2600/106; G01N 2800/102

(57) ABSTRACT

Provided herein are systems and methods for identifying a disease or disorder of a patient, identifying if a patient is likely to respond to a treatment for the disease or disorder, and/or predicting the clinical outcome of the disease or disorder of a patient. Systems and methods described herein may be directed to patients with different chronic conditions, inflammatory conditions, and/or autoimmune conditions. Systems and methods described herein may be directed to patients with arthritis. Systems and methods described herein may comprise analyzing a data set comprising or derived from gene expression data from at least 2 genes, where the gene expression data results from assaying a biological sample from a patient.

### CROSS-REFERENCE

This application claims the benefit of U.S. Provisional Application No. 63/727,799, filed Dec. 4, 2024, which is incorporated herein by reference in its entirety.

### BACKGROUND

Many systemic diseases, such as chronic, inflammatory, and/or autoimmune diseases, are heterogeneous in nature, and have variable causation, course and responsiveness to therapy. At least for these reasons, clinically relevant methods for determining a progression of a disease or disorder as well as the effectiveness of treatments are being explored. Understanding molecular mechanisms of disease variation and sorting patients based on underlying molecular mechanisms can be useful in developing targeted personalized therapy.

### SUMMARY

The present disclosure relates generally to compositions, systems, devices, kits, and methods for disease prediction. Methods for disease prediction comprise methods for predicting the clinical outcome of a patient. Methods described herein comprise treating, preventing, or inhibiting a disease or disorder in a patient.

The present disclosure relates generally to compositions, systems, devices, kits, and methods for disease prediction, and uses thereof. In general, compositions, systems, devices, kits, and methods described herein may be useful in developing targeted personalized treatment for patients with chronic, inflammatory, and/or autoimmune diseases. In some embodiments, methods for treating, preventing, or inhibiting a disease or disorder in a patient comprise identifying subsets of patients or different disease phenotypes as related to gene expression data. In some embodiments, methods for treating, preventing, or inhibiting a disease or disorder in a patient comprise disease prediction of different subsets of patients as related to gene expression data. In some embodiments, methods for predicting the clinical outcome of a patient comprise identifying subsets of patients based on gene expression data. In some embodiments, methods described herein comprise complex data analysis for disease prediction. In some embodiments, disease prediction involves machine learning models, algorithms, and/or classifiers.

### Certain Embodiments

Provided herein are methods comprising: assaying an isolated biological sample from a patient to generate a data set comprising gene expression data, the assaying comprising: (a) performing an analysis with a microarray thereby measuring a concentration of a nucleic acid sequence from the biological sample or an amplicon thereof; (b) performing an RNA-Seq analysis to analyze the transcriptome of a biological sample by sequencing a complementary DNA (cDNA) synthesized from a nucleic acid sequence (RNA) from the biological sample or an amplicon thereof; or (c) quantitative polymerase chain reaction (qPCR) to measure the enrichment of a nucleic acid sequence from the biological sample or an amplicon thereof; and using a computer comprising a non-transitory computer-readable storage media encoded with a computer program including instructions executable by a processor to run an application for identifying and comparing (i) the gene expression data generated from assaying the isolated biological sample to (ii) a reference gene expression data from one or more gene modules; electronically outputting a report detailing the comparison of (i) the gene expression data set generated from assaying the isolated biological sample to (ii) the reference gene expression data set from one or more gene modules; wherein the report: (i) identifies an immunological state of the patient at an accuracy of at least about 70%; (ii) identifies a disease or disorder or a susceptibility thereof of the patient at an accuracy of at least about 70%; (iii) identifies if the patient is likely to respond to a treatment comprising administration of a drug comprising a immunoregulator, a immunosuppressant, a steroid, an anti-inflammatory, a JAK inhibitor, a TNF inhibitors, a baricitinib, a corticosteroid, a nonsteroidal anti-inflammatory drug (NSAID), a tofacitinib, a upadacitinib, a deucravacitinib, a brepocitinib, a disease-modifying anti-rheumatic drug (DMARD), a conventional synthetic DMARD (csDMARD), a targeted synthetic DMARD (tsDMARD), a biologic DMARD (bDMARD), a biologic treatment, a TYK2 inhibitor, a TYK2/JAK inhibitor, a combination inhibitor, a monoclonal antibody, an anti-TNF biologic, an anti-IL-6 biologic, an anti-IL-17 biologic, an anti-IL-12/23 biologic, an anti-CD28 biologic, or combinations thereof; and/or (iv) identifies an effectiveness of the treatment of the patient as compared to the disease or disorder, or the progression of the disease or disorder; wherein: one or more gene modules comprise genes with similar gene expression data, similar gene expression profile, or coexpression; one or more gene modules comprise genes associated with a disease or disorder; each of the one or more gene modules comprise 2 or more genes as shown in TABLE 2, TABLE 3, TABLE 4, or TABLE 5; each of the one or more gene modules are associated with a different disease or disorder, treatment, process, or cell type as shown in FIG. 7, FIG. 8, FIG. 9, FIG. 10, FIG. 11, or FIG. 12; or the disease or disorder is a chronic condition, an inflammatory condition, an autoimmune condition, an arthritis, a rheumatoid arthritis (RA), an early inflammatory arthritis (EIA), an inflammatory arthritis, a psoriatic arthritis (PSA), a lupus arthritis, a rhupus, an osteoarthritis, a non-inflammatory arthritis, a pausi-inflammatory arthritis, or combinations thereof; the isolated biological sample is a whole blood (WB) sample, a peripheral blood mononuclear cell (PBMC) sample, a synovial sample, a tissue sample, a purified cell sample, or combinations thereof; and optionally wherein the method comprising assaying an isolated biological sample from a patient further comprises purifying the isolated biological sample to obtain a purified cell sample.

In some embodiments, a gene module of the one or more gene modules comprises 2 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, or 50 or more genes associated with the gene module. In some embodiments, a gene module of the one or more gene modules comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 genes associated with a biological pathway. In some embodiments, the disease or disorder is the chronic condition. In some embodiments, the disease or disorder is the inflammatory condition. In some embodiments, the disease or disorder is the autoimmune condition. In some embodiments, the disease or disorder is the arthritis. In some embodiments, the disease or disorder is rheumatoid arthritis. In some embodiments, the disease or disorder is early inflammatory arthritis. In some embodiments, the disease or disorder is inflammatory arthritis. In some embodiments, the disease or disorder is psoriatic arthritis. In some embodiments, the disease or disorder is lupus arthritis. In some embodiments, the disease or disorder is rhupus. In some embodiments, the disease or disorder is osteoarthritis. In some embodiments, the disease or disorder is non-inflammatory arthritis. In some embodiments, the disease or disorder is pausi-inflammatory arthritis. In some embodiments, the one or more gene modules are associated with a molecular endotype of a disease or disorder. In some embodiments, the one or more gene modules are associated with a phenotype. In some embodiments, the treatment comprises administration of a drug to the patient. In some embodiments, the treatment comprises parenteral administration of a drug to the patient. In some embodiments, the treatment comprises oral administration of a drug to the patient. In some embodiments, the treatment comprises administration for at least zero weeks, 16 weeks, and 52 weeks, at least 1 year, at least 2 years, at least 3 years, at least 4 years, at least 5 years, at least 6 years, at least 7 years, at least 8 years, at least 9 years, 10 years, at least 15 years, at least 20 years, at least 30 years, at least 35 years, at least 40 years, at least 45 years, at least 50 years, or at least the patient lifespan. In some embodiments, the treatment is adjusted as a function of the gene expression data. In some embodiments, the gene expression data is used to identify a drug for the treatment of the disease or disorder. In some embodiments, the report comprises nucleic acid sequencing data, transcriptome data, genome data, epigenetic data, proteome data, metabolome data, virome data, metabolome data, methylome data, lipidomic data, lineage-ome data, nucleosomal occupancy data, a genetic variant, a gene fusion, an indel, or combinations thereof. In some embodiments, the report comprises different formats. In some embodiments, the report comprises data from different sources, different studies, or combinations thereof. In some embodiments, the data is used to define a phenotype. In some embodiments, the phenotype is associated with a disease or disorder, a progression of a disease or disorder, a trajectory of a disease or disorder, a deterioration, a disability, an organ involvement, a medication response, a treatment response, a treatment target, a treatment, a treatment recommendation, a molecular endotype, or combinations thereof. In some embodiments, the phenotype is associated with a treatment adjusted as a function of a progression of a disease or disorder. In some embodiments, the progression of the disease or disorder comprises rate of deterioration of the patient over time. In some embodiments, the phenotype is associated with a treatment adjusted as a function of a trajectory of a disease or disorder. In some embodiments, the trajectory of the disease or disorder comprises a sequence of diagnoses over time. In some embodiments, the trajectory of the disease or disorder comprises patient decline comprising slow decline, gradual decline, stair step decline, or rapid decline. In some embodiments, the phenotype is associated with a treatment adjusted as a function of the progression of the disease or disorder comprising inflammation, deterioration of a joint, deterioration of a bone, deterioration of a system or structure, and/or disability. In some embodiments, the phenotype is associated with a treatment adjusted to comprise a different rate of administration, a different frequency of administration, administration of a different a drug, administration of more than one drug, administration of different combinations of drugs, or combinations thereof. In some embodiments, the one or more gene modules are a plurality of gene modules. In some embodiments, the method identifies more than one disease or disorder. In some embodiments, the method identifies a first disease or disorder, a second disease or disorder, and/or a third disease or disorder. In some embodiments, the first disease or disorder is different from the second disease or disorder, the first disease or disorder is different from the third disease or disorder, and the second disease or disorder is different from the third disease or disorder.

Provided herein are kits for performing any of the methods described herein, the kit comprising a structural component as well as a composition component, wherein the composition component is a reaction mixture comprising an isolated biological sample from a patient and composition components and/or reagents for performing the assaying. In some embodiments, the structural component comprises a sample interface. In some embodiments, the structural component comprises a computer comprising a non-transitory computer-readable storage media encoded with a computer program including instructions executable by a processor to run an application for identifying and comparing (i) the gene expression data generated from assaying the isolated biological sample to (ii) the reference gene expression data from one or more gene modules.

Provided herein are methods for predicting a clinical outcome of a disease or disorder of a patient, the method comprising: (a) obtaining a first isolated biological sample from a first patient in a first patient population; (b) assaying the first isolated biological sample to generate a data set comprising gene expression data, the assaying comprising: (i) performing an analysis with a microarray thereby measuring a concentration of a nucleic acid sequence from the isolated biological sample or an amplicon thereof; (ii) performing an RNA-Seq analysis to analyze the transcriptome of the isolated biological sample by sequencing a complementary DNA (cDNA) synthesized from a nucleic acid sequence (RNA) from the isolated biological sample or an amplicon thereof; or (iii) performing quantitative polymerase chain reaction (qPCR) to measure the enrichment of a nucleic acid sequence in the isolated biological sample or an amplicon thereof; (c) obtaining a second isolated biological sample from a second patient in a first patient population; (d) assaying the second isolated biological sample to generate a data set comprising gene expression data, the assaying comprising: (i) performing an analysis with a microarray thereby measuring a concentration of a nucleic acid sequence from the isolated biological sample or an amplicon thereof; (ii) performing an RNA-Seq analysis to analyze the transcriptome of the isolated biological sample by sequencing a complementary DNA (cDNA) synthesized from a nucleic acid sequence (RNA) from the isolated biological sample or an amplicon thereof; or (iii) performing quantitative polymerase chain reaction (qPCR) to measure the enrichment of a nucleic acid sequence in the isolated biological sample or an amplicon thereof; (e) using a computer comprising a non-transitory computer-readable storage media encoded with a computer program including instructions executable by a processor to run an application for identifying and comparing the data set comprising the gene expression data of the first patient in the first patient population to the gene expression data of the second patient in the first patient population; (f) defining a signature that is predictive of transcript levels that indicate the clinical outcome of the disease or disorder of the patient from a comparison of the data set comprising the gene expression data of the first patient in the first patient population to the gene expression data of the second patient in the first patient population; (g) electronically outputting a report detailing the signature; wherein the first patient population comprises at least two subsets of patients, each of the at least two subsets of patients corresponding to a different disease phenotype of an established disease or disorder; wherein the first patient in the first patient population comprises a different disease phenotype than the second patient in the first patient population; wherein gene expression data comprises: (i) gene expression data of at least 2 genes of an isolated biological sample from a patient in a patient population; (ii) gene expression data of at least 2 genes associated with one or more gene modules of the isolated biological sample from the patient in the patient population; or (iii) gene expression data of at least 2 genes associated with one or more significant gene modules of the isolated biological sample from the patient in the patient population; wherein the disease or disorder comprises a chronic condition, an inflammatory condition, an autoimmune condition, an arthritis, a rheumatoid arthritis (RA), an early inflammatory arthritis (EIA), an inflammatory arthritis, a psoriatic arthritis (PSA), a lupus arthritis, a rhupus, an osteoarthritis, a non-inflammatory arthritis, a pauci-inflammatory arthritis, or combinations thereof; wherein the first and/or second isolated biological sample is obtained/assayed in intervals of about 3 months or 6 months during at least 10 years; wherein the first and/or second isolated biological sample comprises a blood sample, a whole blood (WB) sample, a peripheral blood mononuclear cell (PBMC) sample, a tissue sample, a synovial sample, a purified cell sample, or combinations thereof; and optionally wherein the method for assaying the first and/or second isolated biological sample comprises purifying the first and/or second isolated biological sample to obtain a purified cell sample. In some embodiments, each of the at least two subsets of patients corresponding to a different disease phenotype respond to a different treatment. In some embodiments, each of the at least two subsets of patients corresponding to a different disease phenotype correlate to a different treatment group. In some embodiments, the comparison of the gene expression data of the first patient in the first patient population to the gene expression data of the second patient in the first patient population is used to identify genetic biomarkers that correlate to a clinical profile of a patient or a clinical outcome of a disease or disorder of a patient. In some embodiments, the gene expression data is systemic data or localized data. In some embodiments, the first isolated biological sample is a blood sample. In some embodiments, the second isolated biological sample is a blood sample. In some embodiments, the first isolated biological sample is a synovial sample. In some embodiments, the second isolated biological sample is a synovial sample. In some embodiments, the first isolated biological sample is a whole blood sample. In some embodiments, the second isolated biological sample is a whole blood sample. In some embodiments, the gene expression data comprises: (i) transcriptomic RNA sequencing data; (ii) RNA expression levels of genes; (iii) RNA expression levels of genes in a gene set or gene module capable of classifying the disease or disorder of a patient; (iii) RNA expression levels of genes in one or more gene modules capable of classifying the disease or disorder of a patient; or (iv) RNA expression levels of genes in one or more gene modules associated with a molecular endotype associated with a disease or disorder. In some embodiments, the gene expression data of at least 2 genes is correlated to one or more gene modules of TABLE 2, TABLE 3, TABLE 4, or TABLE 5. In some embodiments, the gene expression data of at least 2 genes is correlated to one or more significant gene modules of TABLE 2, TABLE 3, TABLE 4, or TABLE 5. In some embodiments, the one or more gene modules are a plurality of gene modules. In some embodiments, the one or more significant gene modules are a plurality of significant gene modules. In some embodiments, the plurality of significant gene modules are as shown in FIG. 7, FIG. 8, FIG. 9, FIG. 10, FIG. 11, or FIG. 12. In some embodiments, the first and/or second isolated biological sample is obtained/assayed during at least 10 years, at least 15 years, at least 20 years, at least 30 years, at least 35 years, at least 40 years, at least 45 years, at least 50 years, at least 55 years, at least 60 years, at least 65 years, at least 70 years, at least 75 years, at least 80 years, at least 85 years, at least 90 years, at least 100 years, or at least the patient lifespan. In some embodiments, the signature indicates clinical outcome. In some embodiments, the signature indicates long-term clinical outcome. In some embodiments, the clinical outcome comprises a treatment. In some embodiments, the treatment comprises administration of a drug to the patient. In some embodiments, the treatment comprises parenteral administration of a drug to the patient. In some embodiments, the treatment comprises oral administration of a drug to the patient. In some embodiments, the treatment is adjusted as a function of the gene expression data. In some embodiments, the gene expression data is used to identify a drug for a treatment of a disease or disorder. In some embodiments, the drug comprises an immunoregulator, an immunosuppressant, a steroid, an anti-inflammatory, a JAK inhibitor, a TNF inhibitor, a baricitinib, a corticosteroid, a nonsteroidal anti-inflammatory drug (NSAID), a tofacitinib, a upadacitinib, a deucravacitinib, a brepocitinib, a disease-modifying antirheumatic drug (DMARD), a conventional synthetic DMARD (csDMARD), a targeted synthetic DMARD (tsDMARD), a biologic DMARD (bDMARD), a biologic treatment, a TYK2 inhibitor, a TYK2/JAK inhibitor, a combination inhibitor, a monoclonal antibody, an anti-TNF biologic, anti-IL-6 biologic, anti-IL-17 biologic, anti-IL-12/23 biologic, anti-CD28 biologic, or combinations thereof. In some embodiments, the signature is: (i) a transcriptomic signature; (ii) predictive of clinical outcomes comprising a treatment of a disease or disorder; or (iii) predictive of clinical outcomes of a disease or disorder of a patient from gene expression data and clinical data, as detailed in FIG. 7, FIG. 8, FIG. 9, FIG. 10, FIG. 11, or FIG. 12. In some embodiments, the report comprises data used to define a phenotype. In some embodiments, the phenotype comprises a disease or disorder, an organ involvement, a medication response, or combinations thereof. In some embodiments, the report further comprises sample trait data. In some embodiments, the sample trait data comprises one or more sample traits listed in TABLE 6. In some embodiments, the sample trait data comprises high sensitivity C-reactive protein (CRP) level, blood C-reactive protein level, blood protein level, blood complement component 3 (C3) protein level, blood complement component 4 (C4) protein level, rheumatoid factor (RF) level, anti-CCP (ACPA) level, matrix metalloproteinase (MMP)-1 level, MMP-3 level, drug level, glucose level, cholesterol level, inflammatory marker level, autoimmune marker level, antibody level, blood autoimmune antibody level, blood pressure, erythrocyte sedimentation rate (ESR), disease activity score (DAS) score, disease activity score for 28 joints (DAS28 score), age, sex, ancestry, drug usage, disease duration, swollen joints, tender joints, tender joint count (TJC), polysymptomatic distress scale (PSD), fibromyalgia score, total areas of pain, or combinations thereof. In some embodiments, the sample trait data is clinical data. In some embodiments, the sample trait data is obtained/assayed in intervals of about 3 months or 6 months during at least 10 years. In some embodiments, the sample trait data is obtained/assayed during at least 10 years, at least 15 years, at least 20 years, at least 30 years, at least 35 years, at least 40 years, at least 45 years, at least 50 years, at least 55 years, at least 60 years, at least 65 years, at least 70 years, at least 75 years, at least 80 years, at least 85 years, at least 90 years, at least 100 years, or at least the patient lifespan. In some embodiments, the report comprising sample trait data is used to identify a progression of a disease or disorder of a patient. In some embodiments, the method for predicting a clinical outcome of a disease or disorder of a patient comprises more than two patients in a first patient population, more than two isolated biological samples, and/or more than two different disease phenotypes. In some embodiments, the first patient population comprises treatment-naïve patients. In some embodiments, the first patient population comprises DMARD-naïve patients. In some embodiments, the first patient population comprises TFi-naïve patients. In some embodiments, the first patient population comprises biologic naïve patients. In some embodiments, the first patient population comprises incomplete responders (IRs). In some embodiments, the first patient population comprises DMARD incomplete responders (DMARD IR), or IR to DMARD treatment. In some embodiments, the first patient population comprises TNF inhibitor incomplete responders (TNFi IR), or IR to TNFi treatment. In some embodiments, the first patient population comprises biologic treatment incomplete responders (biologic IR), or IR to biologic treatment. In some embodiments, the method identifies more than one disease or disorder. In some embodiments, the method identifies a first disease or disorder, a second disease or disorder, and/or a third disease or disorder. In some embodiments, the first disease or disorder is different from the second disease or disorder, the first disease or disorder is different from the third disease or disorder, and the second disease or disorder is different from the third disease or disorder. In some embodiments, the gene expression data of at least 2 genes is correlated to one or more gene modules associated with a molecular endotype of a disease or disorder. In some embodiments, the gene expression data of the one or more gene modules determines a molecular endotype of the patient out of a group of molecular endotypes associated with a disease or disorder, and optionally wherein classifying the disease or disorder of the patient comprises determining the molecular endotype of the patient. In some embodiments, the molecular endotype is indicative of: (i) a subtype of the disease or disorder, (ii) a cellular pathway related to the disease or disorder, (iii) a disease susceptibility of the patient to the disease or disorder; (iv) a treatment with the highest probability of success for the patient; or combinations thereof. In some embodiments, the treatment with the highest probability of success for the patient relates to the treatment that is the most likely to prevent the disease or disorder, reduce a rate of deterioration related to the disease or disorder, reduce or stop a progression of the disease or disorder, reduce or stop a symptom of the disease or disorder, increase a rate of regeneration of the patient as related to the disease or disorder, or combinations thereof. In some embodiments, the treatment with the highest probability of success is the most effective treatment.

Provided herein are kits for performing the method for predicting a clinical outcome of a disease or disorder of a patient of any of the methods described herein, the kit comprising a structural component as well as a composition component, wherein the composition component is a reaction mixture comprising a first isolated biological sample from a first patient in a first patient population and/or a second isolated biological sample from a second patient in a first patient population and composition components and/or reagents for performing the assaying. In some embodiments, the structural component comprises a sample interface. In some embodiments, the structural component comprises a computer comprising a non-transitory computer-readable storage media encoded with a computer program including instructions executable by a processor to run an application for identifying and comparing the data set comprising the gene expression data of the first patient in the first patient population to the gene expression data of the second patient in the first patient population.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1: Sunburst diagram showing ILLUM-1 top5k rowVar genes grouped by coexpression using CodeR-BP.

FIG. 2: Overlap of third generation modules (obtained from grouping ILLUM-1 top5k rowVar genes) with Ampel LuGENE gene function signature list.

FIG. 3A-FIG. 3B: FIG. 3A. Sunburst diagram showing ILLUM-1 top5K rowVar gene modules' eigengenes (ME) correlations to anti-dsDNA (yes=1 if assay >30 IU/mL else no=0). FIG. 3B. Sunburst diagram showing significant overlap (Fisher's adj.p<0.05 and >=4 gene overlaps) of ILLUM-1top5K rowVar gene modules with LuGENE functional characterization groups.

FIG. 4: Correlation of the significant genes modules (obtained from grouping inflammatory arthritis-top5k rowVar genes) with cohort (more inflammatory arthritis) and serologic features of inflammation including erythrocyte sedimentation rate (ESR) and C-Reactive Protein level (CRP). FIG. 4-1. Magnified, top portion of the right vertical axis of FIG. 4. FIG. 4-2. Magnified, bottom portion of the right vertical axis of FIG. 4. FIG. 4-3. Magnified, top portion of the left vertical axis of FIG. 4. FIG. 4-4. Magnified, bottom portion of the left vertical axis of FIG. 4.

FIG. 5: An example of a computer system 1101 that is programmed or otherwise configured to implement methods provided herein.

FIG. 6A-FIG. 6C: RLR patient scoring highly correlates to clinical findings. FIG. 6A: Correlations depicted amongst top clinical attributes of interest. FIG. 6B-FIG. 6C: Correlation of RLR to PSD score (r=0.791, p<0.0001), including the confidence interval (FIG. 6B) and the same for correlation of RLR to SLEDAI (r=−0.746, p<0.0001, FIG. 6C).

FIG. 7, shows heatmap of four patient subsets identified by K-means clustering of 97 RA samples using different immune cell type and process modules (e.g., 32 LUGENE modules (IFN to T reg), IL23 complex, JAK1, JAK2, TYK2, GC (glucocorticoid) in vitro, core GC in vitro, Hu GC, Northcott GC). The heatmap visualizes the GSVA enrichment scores of various immune cell type and process modules, with each column representing a sample and each row corresponding to a different immune cell type and process module. Columns of patients were clustered using idealized k-means clustering and four different disease phenotypes or clusters were identified (i.e., cluster 0, cluster 1, cluster 2, cluster 3).

FIG. 8, shows heatmap of five patient subsets identified by K-means clustering of 378 RA DMARD-IR (Incomplete Response) patients as compared to 20 healthy patients (control) using different immune cell type and process modules (e.g., Inflammation modules, cell modules, JAK1 modules, JAK2 modules, TYK2 modules, Steroid modules). The heatmap visualizes the GSVA enrichment scores of various immune cell type and process modules, with each column representing a sample and each row corresponding to a different immune cell type and process module. Columns of patients were clustered using idealized k-means clustering and five different disease phenotypes or clusters were identified (i.e., cluster 0, cluster 1, cluster 2, cluster 3, cluster 4).

FIG. 9, shows heatmap of five patient subsets identified by K-means clustering of 115 RA TNF-IR (Incomplete Response) patients as compared to 20 healthy patients (control) using different immune cell type and process modules (e.g., Inflammation modules, cell modules, JAK1 modules, JAK2 modules, TYK2 modules, Steroid modules). The heatmap visualizes the GSVA enrichment scores of various immune cell type and process modules, with each column representing a sample and each row corresponding to a different immune cell type and process module. Columns of patients were clustered using idealized k-means clustering and five different disease phenotypes or clusters were identified (i.e., cluster 0, cluster 1, cluster 2, cluster 3, cluster 4).

FIG. 10, shows heatmap of three patient subsets identified by K-means clustering of synovial tissue samples of 46 RA DMARD-IR (Incomplete Response) patients before being treated with a TNF inhibitor (TNFi) using various immune cell type and process modules (e.g., Endotype modules: A, B, C, D, E, F, and G). Overall, FIG. 10 shows clinical response of RA DMARD-IR patients treated with TNFi. As shown in FIG. 10A, with each column representing a sample and each row corresponding to various EULAR response criteria, the heatmap visualizes DMARD-IR patients did not respond to TNFi treatment with no color intensity, DMARD-IR patients were moderate responders to TNFi treatment with low color intensity, DMARD-IR patients were high responders to TNFi treatment with high color intensity. As shown in FIG. 10B, the heatmap visualizes the GSVA enrichment scores of various immune cell type and process modules, with each column representing a sample and each row corresponding to a different module (e.g., Endo modules: A, B, C, D, E, F, and G). Also as shown in FIG. 10A and FIG. 10B, columns of patients were clustered using idealized k-means clustering and three different disease phenotypes or clusters were identified (i.e., cluster 1, cluster 2, cluster 3). Overall, FIG. 10C details correlations between the various immune cell type and process modules (Endo modules) and the clinical criteria.

FIG. 11, shows heatmap of three patient subsets identified by K-means clustering of synovial tissue samples of 46 RA DMARD-IR patients treated with TNFi using various immune cell type and process modules (e.g., Synovium modules: A, B, C, D, E, F, G, H, I, J, K, and L). Overall, FIG. 11 shows clinical response of RA DMARD-IR patients treated with TNFi. As shown in FIG. 11A, with each column representing a sample and each row corresponding to various EULAR response criteria, the heatmap visualizes DMARD-IR patients did not respond to TNFi treatment with no color intensity, DMARD-IR patients were moderate responders to TNFi treatment with low color intensity, DMARD-IR patients were high responders to TNFi treatment with high color intensity. As shown in FIG. 11B, the heatmap visualizes the GSVA enrichment scores of various immune cell type and process modules, with each column representing a sample and each row corresponding to a different module (e.g., Synovium modules: A, B, C, D, E, F, G, H, I, J, K, and L). Also as shown in FIG. 11A and FIG. 11B, columns of patients were clustered using idealized k-means clustering and three different disease phenotypes or clusters were identified (i.e., cluster 1, cluster 2, cluster 3). Overall, FIG. 11C details correlations between the various immune cell type and process modules (Synovium modules) and the clinical criteria.

FIG. 12, shows heatmap of three patient subsets identified by K-means clustering of synovial tissue samples of 46 RA DMARD-IR patients before being treated with TNFi using various immune cell type and process modules (e.g., MEGENA modules: A, B, C, D, E, F, and G). Overall, FIG. 12 shows clinical response of RA DMARD-IR patients treated with TNFi. As shown in FIG. 12A, with each column representing a sample and each row corresponding to various EULAR response criteria, the heatmap visualizes DMARD-IR patients did not respond to TNFi treatment with no color intensity, DMARD-IR patients were moderate responders to TNFi treatment with low color intensity, DMARD-IR patients were high responders to TNFi treatment with high color intensity. As shown in FIG. 12B, the heatmap visualizes the GSVA enrichment scores of various immune cell type and process modules, with each column representing a sample and each row corresponding to a different module (e.g., MEGENA modules: A, B, C, D, E, F, and G). Also as shown in FIG. 12A and FIG. 12B, columns of patients were clustered using idealized k-means clustering and three different disease phenotypes or clusters were identified (i.e., cluster 1, cluster 2, cluster 3). Overall, FIG. 12C details correlations between the various immune cell type and process modules (Synovium modules) and the clinical criteria.

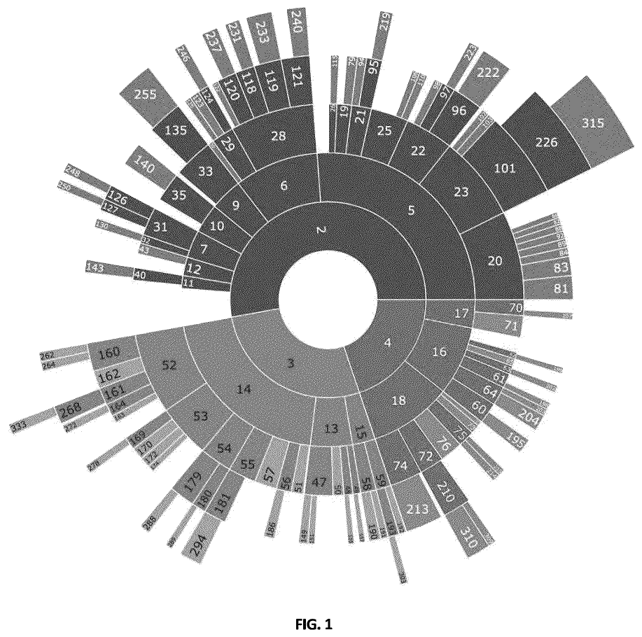

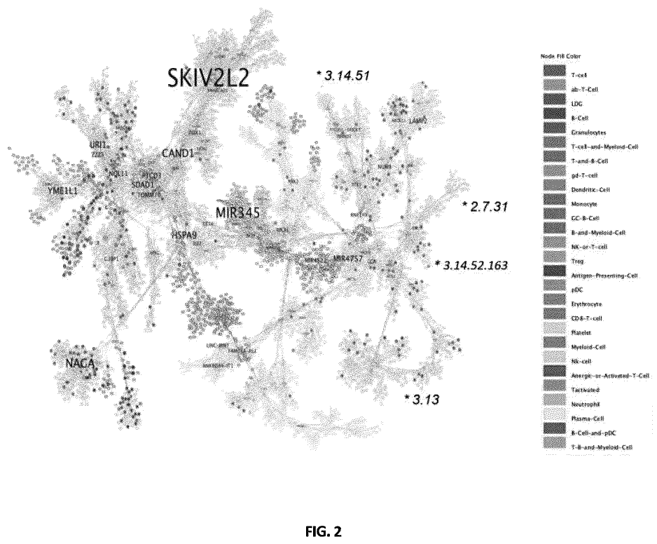

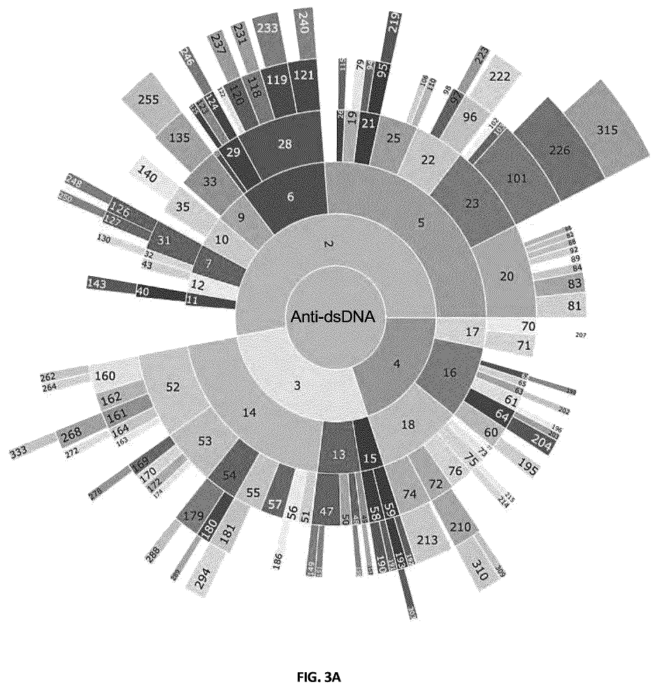

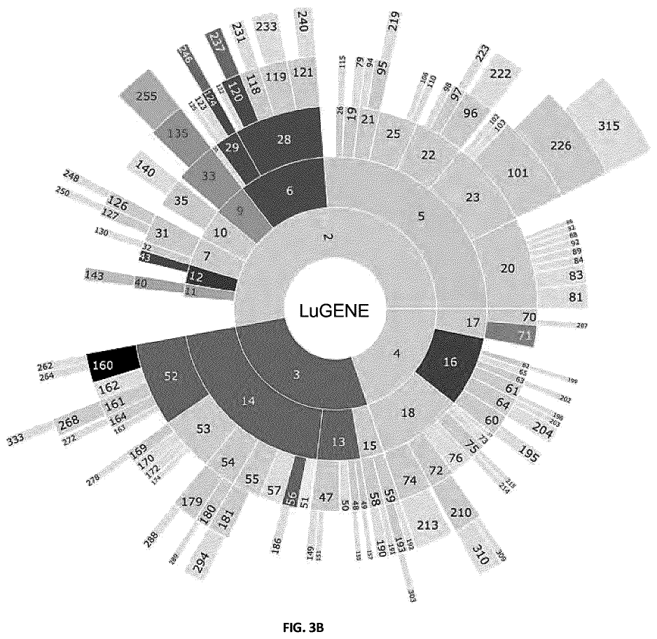

### DETAILED DESCRIPTION

It is to be understood that both the foregoing general description and the following detailed description are exemplary, and explanatory only, and are not restrictive of the disclosure.

The section headings used herein are for organizational purposes only and are not to be construed as limiting the subject matter described.

All documents, or portions of documents, cited in this application, including, but not limited to, patents, patent applications, articles, books, and treatises, are hereby expressly incorporated by reference in their entirety for any purpose.

### Definitions

Unless otherwise indicated, all technical terms used herein have the same meaning as commonly understood by one of ordinary skill in the art to which this invention belongs. Unless otherwise indicated or obvious from context, the following terms have the following meanings:

The terms, “a,” “an,” and “the,” as used herein, comprise plural references unless the context clearly dictates otherwise.

The terms, “or” and “and/or,” as used herein, comprise any and all combinations of one or more of the associated listed items.

The terms, “including,” “comprises,” “comprised,” and other forms, are not limiting.

The terms, “comprise” and its grammatical equivalents, as used herein, specify the presence of stated features, integers, steps, operations, elements, and/or components, but do not preclude the presence or addition of one or more other features, integers, steps, operations, elements, components, and/or groups thereof.

The term, “about,” as used herein in reference to a number or range of numbers, is understood to mean the stated number and numbers +/−10% thereof, or 10% below the lower listed limit and 10% above the higher listed limit for the values listed for a range.

The term, “disease,” as used herein, refers to comprising pathway conditions or pathway systems that are not conducive to cell survival, tissue survival, systemic survival, or organism survival. In some instances, the term “disease” may be used interchangeably with the term “disease state” or “disease or disorder”.

The term, “nucleic acid,” as used herein, refers to a polymer of nucleotides. A nucleic acid may comprise ribonucleotides, deoxyribonucleotides, combinations thereof. A nucleic acid may be single-stranded or double-stranded, unless specified. Non-limiting examples of nucleic acids are double stranded DNA (dsDNA), single stranded (ssDNA), messenger RNA, genomic DNA, and cDNA.

Accordingly, nucleic acids as described herein may comprise one or more mutations, one or more irregularities, or both.

The term “patient” as used herein, refers to an animal. The term “patient” may be used interchangeably with the term “subject”, “test subject”, “reference subject”, “patient”, “test patient” or “reference patient”. In some instances, the patient or subject is a mammal. In some instances, the patient or subject is a human. In some instances, the patient or subject is diagnosed or at risk for a disease. In some instances, the patient or subject is diagnosed or at risk of more than one disease. In some instances, the patient or subject has not received treatment for the disease. In some instances, the patient or subject has received treatment for the disease.

The term, “sample,” as used herein, refers to something comprising a gene of interest. In some instances, the sample is a biological sample, such as a biological fluid or tissue sample. In some instances, the sample is a biological sample isolated from a patient or subject. In some instances, the sample is a biological sample or environmental sample that is modified or manipulated. By way of non-limiting example, samples may be modified or manipulated with purification techniques, digestion techniques, heat, nucleic acid amplification, salts and buffers. In some instances, purification of a sample results in a purified cell sample.

The use of the term “set” (e.g., “a set of items”) or “subset” unless otherwise noted or contradicted by context, is to be construed as a collection comprising one or more members.

The terms, “treatment” and “treating,” as used herein, refer to a pharmaceutical or other intervention regimen for obtaining beneficial or desired results in the patient or subject receiving the treatment. Beneficial or desired results comprise but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the patient, notwithstanding that the patient may still be afflicted with the underlying disorder. A prophylactic effect comprises delaying, preventing, or eliminating the appearance of a disease or condition, delaying, or eliminating the onset of symptoms of a disease or condition, slowing, halting, or reversing a progression of a disease or disorder, or any combination thereof. For prophylactic benefit, a patient at risk of developing a particular disease, or to a patient reporting one or more of the physiological symptoms of a disease may undergo treatment, even though a diagnosis of this disease may not have been made. By way of non-limiting example, a treatment comprises a therapy. In some instances, the treatment comprises personalized therapy. In some instances, the treatment results in therapeutic benefits that are conducive to cell survival, tissue survival, systemic survival, or organism survival.

Various terms used throughout the present description may be read and understood as follows, unless the context indicates otherwise: “or” as used throughout is inclusive, as though written “and/or”; singular articles and pronouns as used throughout comprise their plural forms, and vice versa; similarly, gendered pronouns comprise their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The terms “embodiments,” “some embodiments,” “preferred embodiments,” “specific embodiments,” “some embodiments,” “an embodiment,” “one embodiment” or “other embodiments” mean that a particular feature, structure, or characteristic described in connection with the embodiments is comprised in at least some embodiments, but not necessarily all embodiments, of the present disclosure.

### Overview

Some aspects of the present disclosure are directed to methods and systems for determining a gene set or gene module capable of classifying a disease or disorder of a patient. In some embodiments, a gene module comprises genes grouped in consideration of gene expression data associated with a disease or disorder. In some embodiments, one or more gene modules comprise genes grouped in consideration of gene expression data associated with a disease or disorder. In some embodiments, one or more gene modules comprise a plurality of gene modules associated with a disease or disorder. The gene modules can be used to classify, and/or treat a disease or disorder of a patient. In some embodiments, classifying a disease or disorder of a patient comprises determining whether that patient has the disease and/or which endotype out of two or more endotypes of the disease the patient has. In some embodiments, methods described herein comprise identifying and/or providing targeted therapy for a patient based on the disease or disorder classification of the patient. In some embodiments, the methods described herein comprise the grouping of genes in consideration of gene expression data and analyzing the gene expression data of the group of genes to determine a gene module. In some embodiments, the methods described herein comprise the grouping of more than one gene module in consideration of the gene expression data of the genes in the gene module. In some embodiments, the methods described herein comprise the use of machine learning methods, machine learning models, data analysis, data categorization, or combinations thereof.

### Assays

Provided herein are assays for the generation of gene expression data from a sample from a patient. In some embodiments, the gene expression data from the sample from the patient is indicative of a disease or disorder. In some embodiments, the gene expression data from the sample from the patient is indicative of the progression of a disease or disorder. In some embodiments, the sample is a biological sample isolated from the patient. In some embodiments, the sample is a whole blood sample, a peripheral blood sample, a peripheral blood mononuclear cell sample, a synovial sample, a tissue sample, a purified cell sample, or combinations thereof. In some embodiments, the assays for the generation of gene expression data from a sample from a patient are gene expression assays.

Also provided herein are methods and systems described herein comprising assays for the generation of gene expression data from the sample from the patient, for accurate, repeatable, real-time, determinations of a disease or disorder in a patient (e.g., classifying a disease or disorder, determining progression of disease or disorder, predicting a clinical outcome of a disease or disorder), useful in developing targeted personalized treatment for patients with chronic, inflammatory, and/or autoimmune diseases as described herein.

### Gene Expression Assays

In some embodiments, systems, compositions, methods, kits, and solutions described herein comprise assaying a biological sample from a patient. In some embodiments, systems, compositions, methods, kits, and solutions described herein comprise generating gene expression data. In some embodiments, methods and systems described herein comprise assaying a biological sample from a patient. In some embodiments, methods and systems described herein comprise assaying a biological sample from a patient to generate gene expression data. In some embodiments, methods and systems described herein comprise assaying an isolated biological sample from a patient to generate a dataset comprising gene expression data. In some embodiments, the methods and systems described herein comprise the use of assays to generate gene expression data. In some embodiments, the methods and systems described herein comprise the use of assays to generate gene expression data from a biological sample from a patient. In some embodiments, the methods and systems described herein comprise the use of assays to generate gene expression data that is indicative of a disease or disorder. In some embodiments, the methods and systems described herein comprise the use of microarray assays, RNA-Seq assays, quantitative polymerase chain reaction (qPCR) assays, or combinations thereof.

### Microarray Assay

In some embodiments, methods and systems described herein comprise the use of microarray assays to generate gene expression data from a biological sample from a patient. In some embodiments, methods and systems described herein comprise the use of microarray assays to generate gene expression data that is indicative of a disease or disorder. In some embodiments, methods and systems described herein comprise the use of microarray assays to generate gene expression data from a biological sample from a patient to identify a disease or disorder in a patient.

In some embodiments, a microarray assay comprises measuring the concentration of a nucleic acid sequence in a biological sample. In some embodiments, a microarray assay comprises binding nucleic acids to a surface to measure the concentration of a nucleic acid sequence in a biological sample. In some embodiments, a microarray assay comprises binding 1000's of nucleic acids to a surface to measure the concentration of a nucleic acid sequence in a biological sample, or an amplicon thereof. In some embodiments, the microarray assay comprises binding 1000's of nucleic acids to a surface to measure the relative concentration of nucleic acid sequences in a mixture via hybridization and subsequent detection of the hybridization events. In some embodiments, the microarray assay comprises a nucleic acid array. In some embodiments, the microarray assay comprises a DNA microarray. In some embodiments, the microarray assay measures the concentration of a nucleic acid sequence of interest in a sample. In some embodiments, the concentration of the nucleic acid sequence of interest is gene expression data.

In some embodiments, the microarray assay comprises the deposit or synthesis of a nucleic acid sequence (e.g., DNA sequence). In some embodiments, the a microarray assay comprises the deposit of a nucleic acid sequence on a surface or synthesis of a nucleic acid sequence on a surface.

In some embodiments, the microarray assay comprises a solution comprising labeled nucleic acids and probes attached to a surface. In some embodiments, the microarray assay comprises the solution comprising labeled nucleic acids and probes attached to a surface, such that the binding of the labeled nucleic acids to the probes attached to the surface results in measuring the concentration of a nucleic acid sequence of interest. In some embodiments, the sample from the patient is processed such that the solution comprising labeled nucleic acids comprises nucleic acids from the patient sample from patient. In general, the microarray assay probes are used to measure the concentration of a nucleic acid sequence in a solution or sample. In some embodiments, the microarray assay comprises different probes used to measure the concentration of different types of nucleic acid sequences in a solution or sample. In some embodiments, the microarray assay probes are used to measure the concentration of different nucleic acid sequences in a solution or sample. In some embodiments, the nucleic acid as described herein is a target nucleic acid. In some embodiments, the nucleic acid from a biological sample as described herein is a target nucleic acid. In some embodiments, the nucleic acid sequence as described herein is a target nucleic acid sequence. In some embodiments, the nucleic acid sequence from a biological sample as described herein is a target nucleic acid sequence.

Provided herein are microarrays or DNA microarrays for measuring gene expression levels. In some embodiments, microarrays or DNA microarrays generate gene expression data. In some embodiments, the microarray assay comprises extracting a nucleic acid from cells in the biological sample from the patient.

In some embodiments, the microarray assay comprises isolating a nucleic acid from cells in the biological sample from the patient. In some embodiments, the microarray assay comprises isolating the nucleic acid from cells, enriching the nucleic acid, and labeling the nucleic acid with fluorescent labels or detectable tags. In some embodiments, the labeled nucleic acid is detectable by measuring detectable signals or fluorescent signals. In some embodiments, the labeled nucleic acid hybridizes to probes attached to the surface as described herein. In some embodiments, the surface as described herein is the surface of the array. In some embodiments, the array is washed to remove sample components that are not bound or did not hybridize. In some embodiments, the sample is labeled and the array is stained with a different label to generate a detectable signal. In some embodiments, detectable signal or fluorescent signal from the labeled nuclei acid is measured by scanning the surface. In some embodiments, the intensity of the signal from the labeled nucleic acid is indicative of the concentration of the labeled nucleic acid. In some embodiments, the intensity of the signal in different areas of the surface, corresponding to different probes bound to different labeled nucleic acids, is a measure of gene expression. In some embodiments, different areas of the surface correspond to a different gene.

In some embodiments, the microarray assay comprises isolating RNA from cells in the biological sample from the patient. In some embodiments, the microarray assay comprises isolating RNA from cells, enriching for mRNA, and optionally amplifying RNA. In some embodiments, the microarray assay comprises isolating RNA from cells and (i) labeling the RNA directly; (ii) converting the RNA to a labeled cDNA; or (iii) converting the RNA to an RNA promoter tailed cDNA which is further converted to cRNA. In some embodiments, the cRNA is labeled cRNA. In some embodiments, labeling the RNA comprises the incorporation of nucleotide labels such as fluorescently labeled nucleotides during synthesis. In some embodiments, labeling the RNA comprises the incorporation of fluorescently labeled nucleotides. In some embodiments, labeling the RNA comprises the incorporation of detectable tags for nucleotide detection.

In some embodiments, the nucleic acid as described herein is a target nucleic acid. In some embodiments, the nucleic acid from a biological sample as described herein is a target nucleic acid. In some embodiments, the nucleic acid sequence as described herein is a target nucleic acid sequence. In some embodiments, the nucleic acid sequence from a biological sample as described herein is a target nucleic acid sequence.

### RNA Sequencing (RNA-Seg)

Provided herein are methods for assaying a biological sample from a patient comprising sequencing a nucleic acid from a patient. In some embodiments, the nucleic acid is an RNA. In some embodiments, the methods for assaying are methods for sequencing RNA or methods of RNA sequencing (RNA-Seq).

In some embodiments, methods for sequencing RNA comprise converting RNA to DNA. In some embodiments, methods for sequencing RNA comprise the use of reverse transcriptase (RT) to synthesize complementary DNA (cDNA) from RNA. In some embodiments, methods for sequencing RNA comprise next generation sequencing (NGS) directed to RNA. In some embodiments, methods for sequencing RNA are transcriptomic techniques. In some embodiments, NGS directed to RNA profiles the transcriptome, measuring the activity of thousands of genes at once using high-throughput techniques. In some embodiments, NGS comprises High-Throughput Sequencing (HTS).

In some embodiments, RNA-Seq is a method of transcriptome profiling comprising NGS. In some embodiments, RNA-Seq comprising high-throughput techniques detects nucleic acids present in low concentrations, and/or nucleic acids comprising polymorphisms, mutations, isoforms, or other variations. In some embodiments, RNA-Seq measures non-coding RNAs such as micro-RNAs. In some embodiments, RNA-Seq comprises the detection of different RNA species, including mRNA, non-coding RNA, pathogen RNA, chimeric gene fusions, transcript isoforms, splice variants, and previously unidentified transcripts. In some embodiments, RNA-Seq comprises a comprehensive view of transcript abundance. In some embodiments, RNA-Seq comprises the detection of rare RNA transcript variants, and supports the detection of mutations and germline variation for thousands of expressed genetic variants. In some embodiments, RNA-Seq comprises the assessment of dynamic changes in gene expression in response to various stimuli. In some embodiments, RNA-Seq comprises the identification of biomarkers and/or molecular signatures associated with a disease or disorder, or a subtype of a disease or disorder. In some embodiments, RNA-Seq comprises the identification of molecular signatures associated with responses to a treatment of a disease or disorder (e.g., administration of a drug, treatment of a disease or disorder). In some embodiments, RNA-Seq comprises the identification of a molecular endotype.

In some embodiments, RNA-Seq is a rapid, precise, quantitative measurement of gene expression to generate gene expression data from a cell in an isolated biological sample from a patient. In some embodiments, RNA-Seq measures the activity of a complete set of a patient's transcribed genes.

In some embodiments, methods for sequencing RNA comprise isolating a nucleic acid from a cell in a biological sample from a patient as described herein. In some embodiments, methods for sequencing RNA comprise isolating the nucleic acid from a cell, enriching the nucleic acid, and optionally amplifying the nucleic acid as described herein. In some embodiments, the methods for sequencing RNA comprise labeling the nucleic acid with fluorescent labels or detectable tags. In some embodiments, the labeled nucleic acid is detectable by measuring detectable signals or fluorescent signals. In some embodiments, a labeled nucleic acid generate a detectable signal. In some embodiments, detectable signal or fluorescent signal from the labeled nucleic acid is measured or quantified as light intensity. In some embodiments, the intensity of the signal from the labeled nucleic acid is indicative of the concentration of the labeled nucleic acid. In some embodiments, the intensity of the signal is a measure of gene expression.

In some embodiments, methods for sequencing RNA comprise isolating RNA from a cell in an isolated biological sample as described herein. In some embodiments, methods for sequencing RNA comprise isolating RNA from a cell, enriching for mRNA, and optionally amplifying RNA. In some embodiments, methods for sequencing RNA comprise isolating RNA from a cell and (i) labeling the RNA directly; (ii) converting the RNA to a labeled cDNA; or (iii) converting the RNA to an RNA promoter tailed cDNA which is further converted to cRNA. In some embodiments, the cRNA is labeled CRNA. In some embodiments, labeling the RNA comprises the incorporation of nucleotide labels such as fluorescently labeled nucleotides during synthesis. In some embodiments, labeling the RNA comprises the incorporation of fluorescently labeled nucleotides. In some embodiments, labeling the RNA comprises the incorporation of detectable tags for nucleotide detection.

In some embodiments, methods for sequencing RNA comprise isolating RNA from an isolated biological sample as described herein. In some embodiments, the isolated biological sample is a blood sample. In some embodiments, the blood sample comprises blood cells. In some embodiments, methods for sequencing RNA comprise isolating RNA from blood cells, enriching for mRNA, and optionally amplifying RNA as described herein. In some embodiments, methods for sequencing RNA comprise isolating RNA from blood cells and (i) labeling the RNA directly; (ii) converting the RNA to a labeled cDNA; or (iii) converting the RNA to an RNA promoter tailed cDNA which is further converted to CRNA. In some embodiments, the cRNA is labeled cRNA. In some embodiments, labeling the RNA comprises the incorporation of nucleotide labels such as fluorescently labeled nucleotides during synthesis. In some embodiments, labeling the RNA comprises the incorporation of fluorescently labeled nucleotides. In some embodiments, labeling the RNA comprises the incorporation of detectable tags for nucleotide detection.

In some embodiments, RNA-Seq comprises: (i) isolating RNA from a cell, (ii) enriching for mRNA, (iii) contacting the mRNA with a reverse transcriptase (RT) which synthesize cDNA using mRNA as a template; (iv) fragmenting mRNA or cDNA to short sequences (about 200-500 base pairs of length); (v) amplifying the short sequences; (vi) sequencing the short sequences to generate reads; (vii) aligning the reads to a reference genome; (viii) determining gene expression as the quantity of reads that map to gene exons, absolute read counts, and/or normalized values such as RPKM (reads per kilobase per million mapped reads). In some embodiments, the sequences of the reads are gene transcripts. In some embodiments, reverse transcriptases (RT) is an enzyme used in RNA sequencing (RNA-Seq) to synthesize a complementary DNA (cDNA) from RNA.

In some embodiments, methods for sequencing RNA from a sample from a patient are performed in combination with methods for sequencing DNA from a sample from the patient. In some embodiments, RNA-Seq is combined with DNA sequencing. In some embodiments, RNA-Seq complements the results of DNA sequencing. In some embodiments, RNA-Seq determines whether a variant increases the risk of a disease or disorder in a patient as realed to a clinical outcome. In some embodiments, RNA-Seq determines whether gene expression profile increases the risk of a disease or disorder in a patient as realed to a clinical outcome. In some embodiments, RNA-Seq determines whether gene expression profile corresponds to a disease or disorder in a patient. In some embodiments, RNA-Seq is used to generate gene expression data as described herein. In some embodiments, RNA-Seq is used to generate gene expression data predictive of a future diagnosis. In some embodiments, RNA-Seq is used for prediagnostic screening. In some embodiments, RNA-Seq is used for early disease detection.

In some embodiments, methods for sequencing RNA are used to generate gene expression data as described herein. In some embodiments, gene expression data comprises: (i) transcriptomic RNA sequencing data; (ii) RNA expression levels of genes; or (iii) RNA expression levels of genes in a gene set or gene module capable of classifying the disease or disorder of a patient; (iii) RNA expression levels of genes in one or more gene modules capable of classifying the disease or disorder of a patient; or (iv) RNA expression levels of genes in one or more gene modules associated with a molecular endotype associated with a disease or disorder. In some embodiments, gene expression data comprises transcriptomic RNA sequencing data. In some embodiments, gene expression data comprises RNA expression levels of genes. In some embodiments, gene expression data comprises RNA expression levels of genes in a gene set or gene module capable of classifying the disease state disease or disorder of a patient. In some embodiments, gene expression data comprises RNA expression levels of genes in one or more gene modules capable of classifying the disease or disorder of a patient. In some embodiments, gene expression data comprises RNA expression levels of genes in one or more gene modules associated with a molecular endotype associated with a disease or disorder.

Provided herein are methods for sequencing RNA to generate gene expression data. In some embodiments, gene expression data is indicative of gene expression levels and/or gene expression patterns. In some embodiments, analysis of gene expression data results in the identification of similar gene expression profiles. In some embodiments, gene expression profiles are patterns of gene expression identified from gene expression data. In some embodiments, analysis of gene expression data for one or more genes results in the identification of similar expression profiles for one or more genes. In some embodiments, the gene expression profiles are associated with a disease or disorder. In some embodiments, the gene expression data is associated with a disease or disorder.

In some embodiments, RNA-Seq is used to generate gene expression data associated with a molecular endotype as described herein. In some embodiments, the molecular endotype is indicative of: (i) a subtype of the disease or disorder, (ii) a cellular pathways related to the disease or disorder, (iii) a disease susceptibility of the patient to the disease or disorder; (iv) a treatment with the highest probability of success for the patient; or combinations thereof.

qPCR

In some embodiments, systems, compositions, methods, kits, and solutions described herein comprise a quantitative polymerase chain reaction (qPCR). In some embodiments, methods and systems described herein comprise the use of a qPCR to generate gene expression data from a biological sample from a patient. In some embodiments, methods and systems described herein comprise the use of qPCR to generate gene expression data that is indicative of a disease or disorder. In some embodiments, methods and systems described herein comprise the use of qPCR to generate gene expression data from a biological sample from a patient to identify a disease or disorder in a patient.

In some embodiments, a qPCR comprises measuring the enrichment of a nucleic acid sequence in a biological sample. In some embodiments, the qPCR comprises measuring the concentration of a nucleic acid (e.g., DNA or RNA). In some embodiments, the qPCR comprises measuring the concentration of a DNA. In some embodiments, the qPCR comprises measuring the concentration of a RNA. In some embodiments, the qPCR measures the concentration of a nucleic acid sequence of interest in a sample. In some embodiments, the concentration of the nucleic acid sequence of interest is gene expression data.

In some embodiments, systems, compositions, methods, kits, and solutions described herein comprise a PCR, quantitative polymerase chain reaction (qPCR), real-Time PCR (qPCR), digital PCT, multiplexed qPCR, or combinations thereof, to generate gene expression data from a biological sample from a patient. In some embodiments, the qPCR comprises the synthesis of a nucleic acid sequence. In some embodiments, the nucleic acid as described herein is a target nucleic acid. In some embodiments, the nucleic acid from a biological sample as described herein is a target nucleic acid. In some embodiments, the nucleic acid sequence as described herein is a target nucleic acid sequence. In some embodiments, the nucleic acid sequence from a biological sample as described herein is a target nucleic acid sequence.

In some embodiments, teal-Time PCR (qPCR) is a process of monitoring a PCR reaction by recording the fluorescence generated at the end of amplification each cycle. In fluorescently multiplexed PCR, detection of multiple target nucleic acid sequences in a single reaction is accomplished by associating each nucleic acid target with a distinct fluorescent tag. In some embodiments, qPCR comprises a precise method for measuring multiple reporter signals during multiplex qPCR reactions.

In some embodiments, systems, compositions, methods, kits, and solutions described herein comprise fluorometric signal engineering for multiplexed qPCR. In some embodiments, methods described herein comprise an amplification signal generated by a polymerase chain reaction (PCR). In some embodiments, the PCR reaction comprises a sample and a reagent mixture for amplifying a nucleic acid from the sample. In some embodiments, the PCR reaction comprises a biological sample comprising a nucleic acid and a reagent mixture for amplifying the nucleic acid. In some embodiments, the PCR reaction comprises a biological sample comprising a target nucleic acid and a reagent mixture for amplifying the target nucleic acid. In some embodiments, the PCR reaction comprises a sample, a buffer solution, a reagent mixture comprising polymerases, primers, nucleotides, dNTPS, and the like. In some embodiments, the PCR reaction comprises a sample comprising a plurality of target nucleic acids, at least a first set of paired amplification oligomers configured to amplify at least a first target nucleic acid sequence, at least a second set of paired amplification oligomers configured to amplify at least a second target nucleic acid sequence, at least a first detectable probe configured to anneal to at least the first target nucleic acid sequence, and at least a second detectable probe configured to anneal to at least the second target nucleic acid sequence. In some embodiments, the at least the first set of paired amplification oligomers and at least the first detectable probe is at a different concentration than at least the second set of paired amplification oligomers and at least the second detectable probe.

In some embodiments, the PCR reaction comprises amplifying the first target nucleic acid sequence. In some embodiments, the PCR reaction comprises amplifying the first target nucleic acid sequence with a polymerase having 5′ to 3′ exonuclease activity in the presence of at least the first target nucleic acid sequence to generate at least a first detectable signal, and in the presence of at least the second target nucleic acid sequence to generate at least a second detectable signal, and wherein the amplifying step causes at least the first detectable probe bound to at least the first target nucleic acid and at least the second detectable probe bound to at least the second target nucleic acid to be degraded by the polymerase and permitting generation of at least the first and at least the second detectable signals.

In some embodiments, the PCR reaction comprises measuring the intensity of at least the first detectable signal and at least the second detectable signal upon initiating of the amplification reaction, measuring the intensity of at least the first detectable signal and at least the second detectable signal upon completion of the amplification reaction, and determining a reaction cycle at which the amplification signal first satisfies an amplification criterion. In some embodiments, the PCR reaction comprises detecting signal peak amounts in the signal, and identifying a magnitude of the amplification signal. In some embodiments, the PCR reaction comprises comparing the reaction cycle and the magnitude of the amplification signal and determining the presence or absence of at least the first target nucleic acid sequence, and the presence or absence of at least the second target nucleic acid sequence in the sample.

In some embodiments, at least the first set of paired amplification oligomers comprises at least a first forward amplification oligomer and at least a first reverse amplification oligomer. In some embodiments, the at least the second set of paired amplification oligomers comprises at least a second forward amplification oligomers and at least a second reverse amplification oligomer. In some embodiments, the at least the first detectable probe is configured to anneal to at least the first target nucleic acid sequence. In some embodiments, the at least the second detectable probe is configured to anneal to at least the second target nucleic acid sequence. In some embodiments, the amplification is a PCR. In some embodiments, the PCR is a qPCR.

In some embodiments, the qPCR comprises a mixture comprising: a) a buffer; b) a salt; c) a set of dNTPs; and d) an enzyme. In some embodiments, the salt is selected from the group consisting of: (a) magnesium chloride; (b) sodium chloride; (c) potassium chloride; or (d) sodium citrate. In some embodiments, the set of dNTPs is selected from the group consisting of: Set A comprising deoxyadenosine triphosphate (dATP), deoxy cytidine triphosphate (dCTP), deoxyguanosine triphosphate (dGTP), deoxythymidine triphosphate (dTTP); and Set B comprising adenosine triphosphate (ATP), cytidine triphosphate (CTP), guanosine triphosphate (GTP) and uridine triphosphate (UTP). In some embodiments, said enzyme is selected from the group consisting of: (a) a thermostable DNA polymerase; (b) a reverse transcriptase; and (c) a RNA polymerase.

In some embodiments, the first detectable probe and at least the second detectable probe are each independently selected from the group consisting of: a dye; a fluorescent molecule; a chemiluminescent label; and a combination thereof. In some embodiments the at least the first detectable probe and at least the second detectable probe is a dye. In some embodiments the at least the first detectable probe and at least the second detectable probe is a fluorescent molecule. In some embodiments the at least the first detectable probe and at least the second detectable probe is a chemiluminescent label. In some embodiments, the method further comprises during said amplification reaction, releasing said dye from said detection probe, thereby generating said at least a first signal and said at least a second signal. In some embodiments, the mixture comprises primers and probes for at least five nucleic acid targets.

### Amplification Reagents/Components

In some embodiments, systems, compositions, methods, kits, and solutions described herein comprise a reagent or component for amplifying a nucleic acid. Non-limiting examples of reagents for amplifying a nucleic acid comprise polymerases, primers, and nucleotides. In some embodiments, systems comprise reagents for nucleic acid amplification in a sample. In some embodiments, nucleic acid amplification improves at least one of sensitivity, specificity, or accuracy of the assay. In some embodiments, nucleic acid amplification is isothermal nucleic acid amplification, providing for the use of the system or system in remote regions or low resource settings without specialized equipment for amplification. In some embodiments, amplification of the nucleic acid increases the concentration of the nucleic acid in the sample. In some embodiments, methods and systems described herein comprise labeling the nucleic acid with fluorescent labels or detectable tags. In some embodiments, labeling the nucleic acid comprises the incorporation of nucleotide labels or fluorescent labels such as fluorescently labeled nucleotides during synthesis. In some embodiments, labeling the nucleic acid comprises the incorporation of fluorescently labeled nucleotides. In some embodiments, labeling the nucleic acid comprises the incorporation of detectable tags for nucleotide detection. In some embodiments, fluorescent labels or detectable tags generate a detectable signal. In some embodiments, a detectable signal is measured with an appropriate detection device (e.g., a fluorescent light reader, a visible light reader).

In some embodiments, the reagents for nucleic acid amplification comprise a recombinase, a primer, an oligonucleotide primer, an activator, a deoxynucleoside triphosphate (dNTP), a ribonucleoside triphosphate (rNTP), a single-stranded DNA binding (SSB) protein, Rnase inhibitor, water, a polymerase, reverse transcriptase mix, or a combination thereof that is suitable for an amplification reaction. Non-limiting examples of amplification reactions are transcription mediated amplification (TMA), helicase dependent amplification (HDA), or circular helicase dependent amplification (cHDA), strand displacement amplification (SDA), recombinase polymerase amplification (RPA), loop mediated amplification (LAMP), exponential amplification reaction (EXPAR), rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), and improved multiple displacement amplification (IMDA).

Such amplification reactions, in some embodiments, are also used in combination with reverse transcription of an RNA of interest. Accordingly, also provided herein are reagents for both the reverse transcription and amplification of nucleic acids. In some embodiments, systems, compositions, methods, kits, and solutions comprise 0.01 μL, 0.02 μL, 0.03 μL, 0.04 μL, 0.05 μL, 0.06 μL, 0.07 μL, 0.08 μL, 0.09 μL, 0.1 μL, 0.2 μL, 0.3 μL, 0.4 μL, 0.5 μL, 0.6 μL, 0.7 μL, 0.8 μL, 0.9 μL, 1 μL, 2 μL, 3 μL, 4 μL, 5 μL, 6 μL, 7 μL, 8 μL, 9 μL, 10 μL, 20 μL, 30 μL, 40 μL, 50 μL, 60 μL, 70 μL, 80 μL, 90 μL, 100 μL, 150 μL, 200 μL, 250 μL, 300 μL, 350 μL, 400 μL, 450 μL, 500 μL, or more of each amplification described herein. In some embodiments, systems, compositions, methods, kits, and solutions comprise 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, or more of each amplification reagent as described herein. In some embodiments, systems, compositions, methods, kits, and solutions comprise 1 μM, 2 μM, 3 μM, 4 μM, 5 μM, 6 μM, 7 μM, 8 μM, 9 μM, 10 μM, 20 μM, 30 M, 40 μM, 50 μM, 60 μM, 70 μM, 80 μM, 90 μM, 100 μM, 150 μM, 200 μM, 250 μM, 300 μM, 350 μM, 400 μM, 450 μM, 500 μM, or more of each amplification reagent as described herein. In some embodiments, systems, compositions, methods, kits, and solutions comprise 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or more of each amplification reagent as described herein.

In some embodiments, methods and systems described herein comprise a PCR tube, a PCR well or a PCR plate. In some embodiments, the wells of the PCR plate are pre-aliquoted with the reagent for amplifying a nucleic acid. In some embodiments, a user thus adds the biological sample of interest to a well of the pre-aliquoted PCR plate and measure for a detectable signal with a fluorescent light reader or a visible light reader.

In some embodiments, systems comprise a PCR plate and a support medium. In some embodiments, nucleic acid amplification is performed in a nucleic acid amplification region on the support medium. Alternatively, or in combination, the nucleic acid amplification is performed in a reagent chamber, and the resulting sample is applied to the support medium.

Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value 1 to 60 minutes. In some embodiments, the amplification reaction is performed for 1 to 60, 5 to 55, 10 to 50, 15 to 45, 20 to 40, or 25 to 35 minutes. In some embodiments, the amplification reaction is performed at a temperature of around 20-45° C. In some embodiments, the amplification reaction is performed at a temperature no greater than 20° C., 25° C., 30° C., 35° C., 37° C., 40° C., 45° C., 50° C., 55° C., 60° C., 65° C. or any value 20° C. to 65° C. In some embodiments, the amplification reaction is performed at a temperature of at least 20° C., 25° C., 30° C., 35° C., 37° C., 40° C., 45° C., 50° C., 55° C., 60° C., 65° C. or any value 20° C. to 65° C. In some embodiments, the amplification reaction is performed at a temperature of 20° C. to 45° C., 25° C. to 40° C., 30° C. to 40° C., 35° C. to 40° C., 40° C. to 45° C., 45° C. to 50° C., 50° C. to 55° C., 55° C. to 60° C., 55° C. to 65° C., or 60° C. to 65° C.

In some embodiments, methods and systems described herein comprise primers for amplifying a nucleic acid to produce an amplification product. The compositions for amplification of nucleic acids and methods of use thereof, as described herein, are compatible with any of the methods disclosed herein including methods of assaying a nucleic acid.

In some embodiments, methods and systems described herein comprise a PCR, qPCR, digital PCT, multiplexed qPCR, or combinations thereof, to generate gene expression data from a biological sample from a patient.

### Samples

In some embodiments, a sample is isolated from a patient. In some embodiments, sample is an isolated biological sample from a patient. In some embodiments, isolating a biological sample from a patient comprises techniques known in the art for the isolation of a biological sample as described herein. In some embodiments, the isolated biological sample is selected from a group consisting of: a whole blood (WB) sample, a peripheral blood mononuclear cell (PBMC) sample, a synovial sample, a tissue sample, and a purified cell sample.

Various sample types are consistent with the present disclosure. In some embodiments, the samples are isolated from a patient for identification of a disease or disorder. In some embodiments, the identification of a disease or disorder comprises identification of the progression of a disease or disorder or a susceptibility thereof of the patient. In some embodiments, the identification of a disease or disorder as described herein comprises the assaying of an isolated biological sample from a patient. In some embodiments, the identification of a disease or disorder as described herein comprises assaying a sample to generate a dataset comprising gene expression data. In some embodiments, the identification of a disease or disorder as described herein comprises assaying for phenotyping, genotyping, or determining patient ancestry. Generally, a sample from a patient is obtained for testing a presence of a disease or disorder, a susceptibility of a patient to a disease or disorder, a progression of a disease or disorder, a response to or effectiveness of a treatment administered to the patient with a disease or disorder, or combinations thereof.

In some embodiments, the sample comprises nucleic acids associated to pathway conditions or pathway systems that are not conducive to cell survival, tissue survival, systemic survival, or organism survival. In some embodiments, the sample comprises nucleic acids associated to a disease or disorder as described herein.

In some embodiments, the sample comprises a cell comprising pathway conditions or pathway systems that are not conducive to cell survival, tissue survival, systemic survival, or organism survival. In some embodiments, the sample comprises cells and/or cell pathway conditions associated to a disease or disorder as described herein.

In some embodiments, samples are used for diagnosing a disease. In some embodiments, the disease is a disease or disorder as described herein.

In some embodiments, a sample is used for identifying a disease status. For example, a sample is any sample described herein, and is obtained from a patient for use in identifying a disease status of a patient. In some embodiments, the disease is a systemic disease or disorder. In some embodiments, the disease is a chronic disease or disorder. In some embodiments, the disease is an inflammatory disease or disorder. In some embodiments, the disease is a non-inflammatory disease or disorder. In some embodiments, the disease is an autoimmune disease or disorder. In some embodiments, the disease is an arthritis disease or disorder. In some embodiments, the disease is a lupus disease or disorder. In some embodiments, a method comprises obtaining a sample from a patient; and identifying a disease status of the patient. In some embodiments, the sample from a patient is a whole blood (WB) sample, a peripheral blood mononuclear cell (PBMC) sample, a blood sample, a synovial sample, a tissue sample, a purified cell sample, or combinations thereof.

In some embodiments, the sample comprises a blood sample, a whole blood (WB) sample, a peripheral blood mononuclear cell (PBMC) sample, an isolated peripheral blood mononuclear cell (PBMCs) sample, a tissue sample, a tissue biopsy sample, a synovial sample, a synovial biopsy sample, a purified cell sample, a nasal fluid sample, a saliva sample, a urine sample, a stool sample, or combinations thereof.

In some embodiments, the sample comprises a blood sample, a whole blood sample (WB), a peripheral blood mononuclear cell (PBMCs) sample, a tissue sample, a synovial sample, a purified cell sample, any derivative thereof, or combinations thereof. In some instances, the blood sample is a whole blood sample or a PBMC sample. In some instances, the blood sample comprises blood cells, serum, plasma, or any combination thereof. In some embodiments, the sample is a biological sample. In some embodiments, the biological sample is a reference biological sample. In some embodiments, the biological sample is an isolated biological sample.

In some embodiments, the isolated biological sample is selected from: a whole blood (WB) sample, a peripheral blood mononuclear cell (PBMC) sample, a blood sample, a synovial sample, a tissue sample, a purified cell sample, or combinations thereof.

In some embodiments, the biological sample is selected from: a blood sample, a whole blood (WB) sample, a peripheral blood mononuclear cell (PBMC) sample, a synovial sample, a synovial biopsy sample, a tissue sample, a tissue biopsy sample, a skin biopsy sample, a purified cell sample, any derivative thereof, or combinations thereof. In some embodiments, the biological sample comprises a blood sample, or any derivative thereof. In some embodiments, the biological sample comprises a whole blood sample, or any derivative thereof. In some embodiments, the biological sample comprises a PBMC sample, or any derivative thereof. In some embodiments, the biological sample comprises a synovial sample, or any derivative thereof. In some embodiments, the biological sample comprises a synovial biopsy sample, or any derivative thereof. In some embodiments, the biological sample comprises a tissue sample, or any derivative thereof. In some embodiments, the biological sample comprises a tissue biopsy sample, or any derivative thereof. In some embodiments, the biological sample comprises a skin biopsy sample, or any derivative thereof. In some embodiments, the biological sample comprises a purified cell sample, or any derivative thereof. In some embodiments, the biological sample is an isolated biological sample.

In some embodiments, isolating a biological sample from a patient comprises techniques known in the art for the isolation of a biological sample as described herein. To isolate a blood sample, various techniques may be used, e.g., a syringe or other vacuum suction device. A blood sample can be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is isolated, or longer if frozen. When isolating a sample from a patient (e.g., blood sample), the amount can vary depending upon patient size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is isolated. In some embodiments, 1-50, 2-40, 3-30, or 4-20 μL of sample is isolated. In some embodiments, more than 5, 10, 15, 20, 25, 30, 35, 40, 45, 50, 55, 60, 65, 70, 75, 80, 85, 90, 95 or 100 μL of a sample is isolated.

In some embodiments, a sample comprises a nucleic acid. In some embodiments, the nucleic acid is from a gene with a mutation associated with a disease or disorder as described herein. In some embodiments, the nucleic acid is from a gene whose expression/overexpression/underexpression is associated with a disease or disorder as described herein. In some embodiments, the nucleic acid is encoded by a nucleic acid sequence associated with a disease or disorder as described herein. In some embodiments, the nucleic acid is an RNA or a complementary DNA synthesized from a nucleic acid (RNA) from at least one gene associated with a disease or disorder as described herein. In some embodiments, the nucleic acid is from a genomic locus, a transcribed mRNA, or a reverse transcribed mRNA, a DNA amplicon or a cDNA from at least one gene associated with a disease or disorder as described herein.

Provided herein are nucleic acids in the sample which are encoded by a nucleic acid sequence. In some embodiments, the nucleic acid sequence from a biological sample as described herein is a target nucleic acid sequence.

In some embodiments, a sample comprises a nucleic acid sequence. In some embodiments, the assaying of the sample generates a dataset comprising gene expression data. In some embodiments, the sample is the isolated biological sample from a patient. In some embodiments, the isolated biological sample from the patient comprises a nucleic acid sequence. In some embodiments, the assaying of the biological sample from the patient generates a dataset comprising gene expression data. In some embodiments, the assaying of the biological sample from the patient comprises measuring a concentration of a nucleic acid sequence. In some embodiments, the assaying of the biological sample from the patient comprises sequencing a nucleic acid sequence from the biological sample or an amplicon thereof. In some embodiments, the assaying of the biological sample from the patient comprises analyzing the transcriptome of the biological sample by sequencing a nucleic acid sequence (e.g., RNA) from the biological sample or an amplicon thereof. In some embodiments, the assaying of the biological sample from the patient comprises analyzing the transcriptome of the biological sample by sequencing a complementary DNA (e.g., cDNA) synthesized from a nucleic acid sequence (e.g., RNA) from the biological sample or an amplicon thereof. In some embodiments, the assaying of the biological sample from the patient comprises measuring the enrichment of the nucleic acid sequence in the biological sample or an amplicon thereof. In some embodiments, the assaying of the biological sample from the patient comprises a microarray assay, an RNA-Seq assay, and/or a quantitative polymerase chain reaction (qPCR).

In some embodiments, a sample comprises a nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 M, less than 6 μM, less than 7 μM, less than 8 μM, less than 9 μM, less than 10 μM, less than 100 M, or less than 1 mM. In some embodiments, the sample comprises a nucleic acid at a concentration of 1 nM to 2 nM, 2 nM to 3 nM, 3 nM to 4 nM, 4 nM to 5 nM, 5 nM to 6 nM, 6 nM to 7 nM, 7 nM to 8 nM, 8 nM to 9 nM, 9 nM to 10 nM, 10 nM to 20 nM, 20 nM to 30 nM, 30 nM to 40 nM, 40 nM to 50 nM, 50 nM to 60 nM, 60 nM to 70 nM, 70 nM to 80 nM, 80 nM to 90 nM, 90 nM to 100 nM, 100 nM to 200 nM, 200 nM to 300 nM, 300 nM to 400 nM, 400 nM to 500 nM, 500 nM to 600 nM, 600 nM to 700 nM, 700 nM to 800 nM, 800 nM to 900 nM, 900 nM to 1 μM, 1 μM to 2 μM, 2 μM to 3 μM, 3 μM to 4 μM, 4 μM to 5 μM, 5 μM to 6 μM, 6 μM to 7 μM, 7 μM to 8 μM, 8 μM to 9 μM, 9 μM to 10 μM, 10 μM to 100 μM, 100 μM to 1 mM, 1 nM to 10 nM, 1 nM to 100 nM, 1 nM to 1 μM, 1 nM to 10 μM, 1 nM to 100 μM, 1 nM to 1 mM, 10 nM to 100 nM, 10 nM to 1 μM, 10 nM to 10 μM, 10 nM to 100 μM, 10 nM to 1 mM, 100 nM to 1 μM, 100 nM to 10 M, 100 nM to 100 μM, 100 nM to 1 mM, 1 μM to 10 μM, 1 μM to 100 μM, 1 μM to 1 mM, 10 μM to 100 μM, 10 μM to 1 mM, or 100 μM to 1 mM. In some embodiments, the sample comprises a nucleic acid at a concentration of 20 nM to 200 μM, 50 nM to 100 μM, 200 nM to 50 μM, 500 nM to 20 μM, or 2 μM to 10 μM. In some embodiments, the sample comprises an amplicon of a nucleic acid. In some embodiments, the nucleic acid is not present in the sample.

In some embodiments, the sample is a biological sample, an environmental sample, or a combination thereof. Non-limiting examples of biological samples are blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, and a tissue sample (e.g., a biopsy sample). In some embodiments, a tissue sample from a patient is dissociated or liquified prior to application to detection system of the present disclosure. Non-limiting examples of environmental samples are soil, air, or water. In some embodiments, an environmental sample is taken as a swab from a surface of interest or taken directly from the surface of interest.

In some embodiments, the sample is a raw (unprocessed, unedited, unmodified) sample. In some embodiments, raw samples are assayed as described herein. In some embodiments, the sample is diluted with a buffer or a fluid or concentrated prior to assaying as described herein. Sometimes, the sample comprises no more 20 μl of buffer or fluid. The sample, in some embodiments, is comprised in no more than 0.01, 0.1, 0.5, 1, 5, 10, 15, 20, 25, 30, 35 40, 45, 50, 55, 60, 65, 70, 75, 80, 90, 100, 200, 300, 400, 500 μl, or any of value 0.01 μl to 500 μl, 0.1 μL to 100 μL, or more preferably 1 μL to 50 μL of buffer or fluid. Sometimes, the sample is comprised in more than 500 μl. In some embodiments, the compositions, systems, and methods disclosed herein are compatible with the buffers or fluid disclosed herein.

In some embodiments, the sample is taken from a single-cell eukaryotic organism; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some embodiments, the sample is taken from nematodes, protozoans, helminths, or malarial parasites. In some embodiments, the sample comprises nucleic acids from a cell lysate from a eukaryotic cell, a mammalian cell, a human cell, a prokaryotic cell, or a plant cell. In some embodiments, the sample comprises nucleic acids expressed from a cell.

### Sample Trait Data

In some embodiments, the methods described herein comprise sample trait data as described herein. In some embodiments, the sample trait data is clinical data. In some embodiments, sample trait data is used to identify a disease or disorder of a patient as described herein. In some embodiments, sample trait data is used to classify a disease or disorder of a patient as described herein. In some embodiments, sample trait data is used to identify a progression of a disease or disorder of a patient. In some embodiments, sample trait data is used to identify a treatment of a disease or disorder of a patient. In some embodiments, sample trait data comprises one or more sample traits described herein. In some embodiments, one or more sample traits are selected from the sample traits described herein. In some embodiments, sample trait data comprises one or more sample traits listed in TABLE 6. In some embodiments, the sample traits are selected from the sample traits listed in TABLE 6. In some embodiments, the one or more sample traits are selected from the sample traits listed in TABLE 6.

In some embodiments, the one or more sample traits comprise blood autoimmune antibody level, SLEDAI score, blood C3 protein level, PSD score, age, ancestry, Hispanic ancestry, African ancestry, NSAIDs usage, prednisone usage, amitriptyline usage, total areas of pain, or combinations thereof. In some embodiments, the one or more sample traits comprise blood autoimmune antibody level, SLEDAI score, blood C3 protein level, PSD score, age, ancestry, or combinations thereof. In some embodiments, the one or more sample traits comprise blood autoimmune antibody level, SLEDAI score, blood C3 protein level, PSD score, immunosuppressive drug usage, duloxetine usage, or combinations thereof. In some embodiments, the one or more sample traits comprise blood autoimmune antibody level.

In some embodiments, the sample trait data comprises one or more sample traits selected from the group consisting of: high sensitivity C-reactive protein (CRP) level, blood C-reactive protein level, blood protein level, blood complement component 3 (C3) protein level, blood complement component 4 (C4) protein level, rheumatoid factor (RF) level, anti-CCP (ACPA) level, matrix metalloproteinase (MMP)-1 level, MMP-3 level, drug level, glucose level, cholesterol level, inflammatory marker level, autoimmune marker level, antibody level, blood autoimmune antibody level, blood pressure, erythrocyte sedimentation rate (ESR), disease activity score (DAS) score, disease activity score for 28 joints (DAS28 score), age, sex, ancestry, drug usage, disease duration, swollen joints, tender joints, tender joint count (TJC), polysymptomatic distress scale (PSD), fibromyalgia score, total areas of pain, and any combination thereof.

In some embodiments, the sample trait data is clinical data. In some embodiments, the sample trait data is obtained/assayed in intervals of about 3 months or 6 months during at least 10 years. In some embodiments, the sample trait data is obtained/assayed during at least 10 years, at least 15 years, at least 20 years, at least 30 years, at least 35 years, at least 40 years, at least 45 years, at least 50 years, at least 55 years, at least 60 years, at least 65 years, at least 70 years, at least 75 years, at least 80 years, at least 85 years, at least 90 years, at least 100 years, or at least the patient lifespan. In some embodiments, the sample trait data is used to identify progression of a disease or disorder of a patient.

### Gene Module

In some embodiments, methods described herein comprise gene modules. In some embodiments, methods described herein comprise gene modules comprising at least 2 genes. In some embodiments, the gene modules described herein comprise at least 2 genes, at least 3 genes, at least 4 genes, at least 5 genes, at least 6 genes, at least 7 genes, at least 8 genes, at least 9 genes, at least 10 genes, at least 11 genes, at least 12 genes, at least 13 genes, at least 14 genes, at least 15 genes, at least 16 genes, at least 17 genes, at least 18 genes, at least 19 genes, at least 20 genes, at least 21 genes, at least 22 genes, at least 23 genes, at least 24 genes, at least 25 genes, at least 26 genes, at least 27 genes, at least 28 genes, at least 29 genes, at least 30 genes, at least 31 genes, at least 32 genes, at least 33 genes, at least 34 genes, at least 35 genes, at least 36 genes, at least 37 genes, at least 38 genes, at least 39 genes, at least 40 genes, at least 41 genes, at least 42 genes, at least 43 genes, at least 44 genes, at least 45 genes, at least 46 genes, at least 47 genes, at least 48 genes, at least 49 genes, or at least 50 genes. In some embodiments, the gene modules described herein comprise at least 2 genes, at least 5 genes, at least 10 genes, at least 15 genes, at least 20 genes, at least 25 genes, at least 30 genes, at least 35 genes, at least 40 genes, at least 45 genes, or at least 50 genes. In some embodiments, the gene modules described herein comprise 2 or more genes, 5 or more genes, 10 or more genes, 15 or more genes, 20 or more genes, 25 or more genes, 30 or more genes, 35 or more genes, 40 or more genes, 45 or more genes, or 50 or more genes. In some embodiments, the gene modules described herein comprise about 2 genes, about 5 genes, about 10 genes, about 15 genes, about 20 genes, about 25 genes, about 30 genes, about 35 genes, about 40 genes, about 45 genes, or about 50 genes. In some embodiments, the gene modules described herein comprise at least 2 genes, at least 5 genes, at least 10 genes, at least 15 genes, at least 20 genes, at least 25 genes, at least 30 genes, at least 35 genes, at least 40 genes, at least 45 genes, or at least 50 genes associated with a disease or disorder described herein. In some embodiments, the gene modules described herein comprise at least 2 genes, at least 5 genes, at least 10 genes, at least 15 genes, at least 20 genes, at least 25 genes, at least 30 genes, at least 35 genes, at least 40 genes, at least 45 genes, or at least 50 genes associated with a molecular endotype described herein. In some embodiments, the gene modules described herein comprise at least 2 genes, at least 5 genes, at least 10 genes, at least 15 genes, at least 20 genes, at least 25 genes, at least 30 genes, at least 35 genes, at least 40 genes, at least 45 genes, or at least 50 genes associated with a biological pathway. In some embodiments, the gene modules described herein comprise at least 2 genes, at least 5 genes, at least 10 genes, at least 15 genes, at least 20 genes, at least 25 genes, at least 30 genes, at least 35 genes, at least 40 genes, at least 45 genes, or at least 50 genes associated with a phenotype. In some embodiments, the phenotype is associated with a disease or disorder, a progression of a disease or disorder, a deterioration, a disability, an organ involvement, a medication response, a treatment response, a treatment target, a molecular endotype, or any combination thereof.

In some embodiments, methods described herein comprise gene modules comprising at least 2 genes associated with a disease or disorder as described herein. In some embodiments, methods described herein comprise gene modules comprising at least 2 genes as described in TABLE 2, TABLE 3, TABLE 4, or TABLE 5. In some embodiments, methods described herein comprise gene modules comprising 2 or more genes as described in TABLE 2, TABLE 3, TABLE 4, or TABLE 5. In some embodiments, the gene modules described herein comprise the genes described in TABLE 2, TABLE 3, TABLE 4, or TABLE 5. In some embodiments, the at least 2 genes as described herein comprise genes described in TABLE 2, TABLE 3, TABLE 4, or TABLE 5.

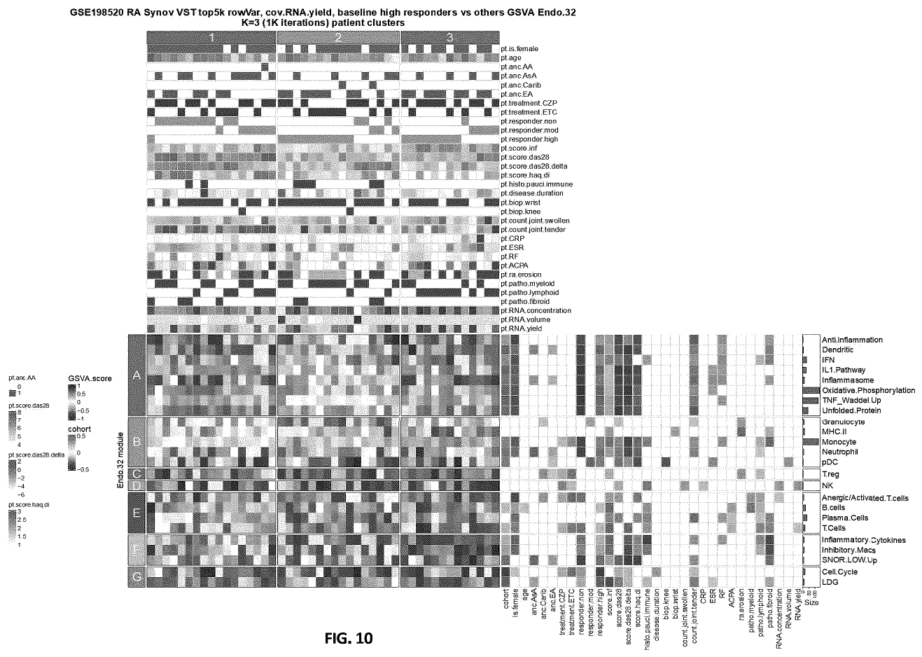

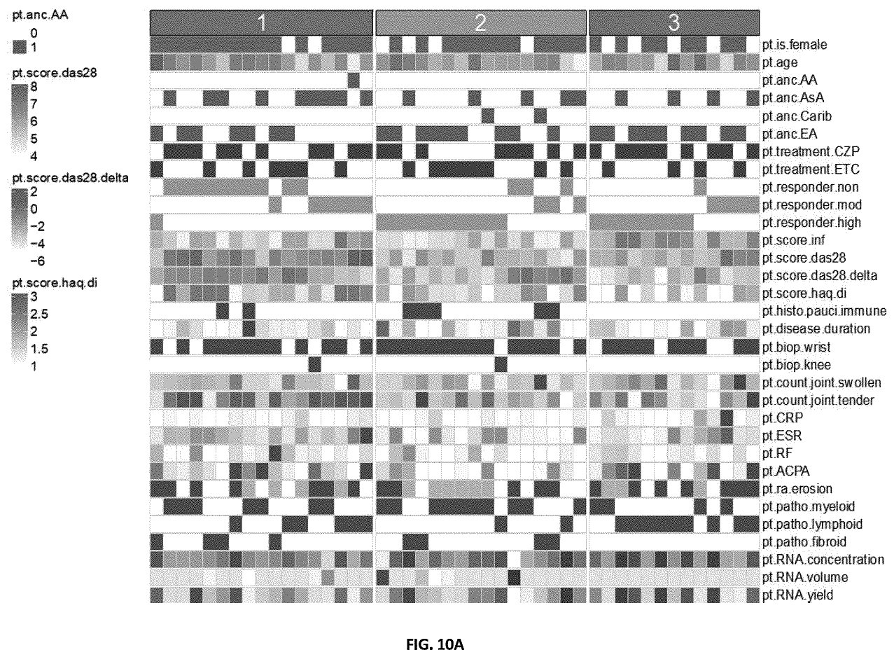

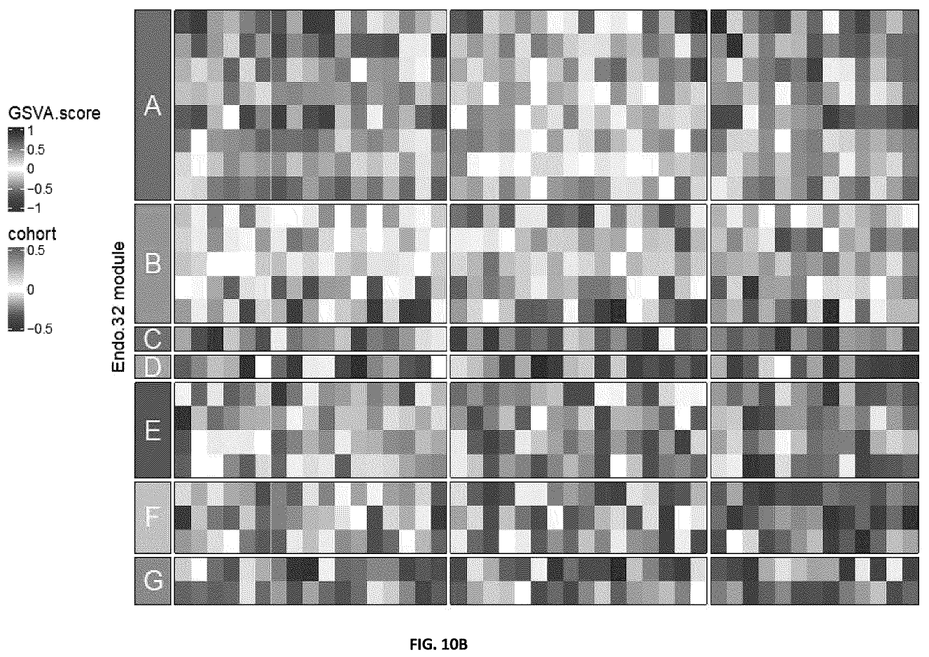

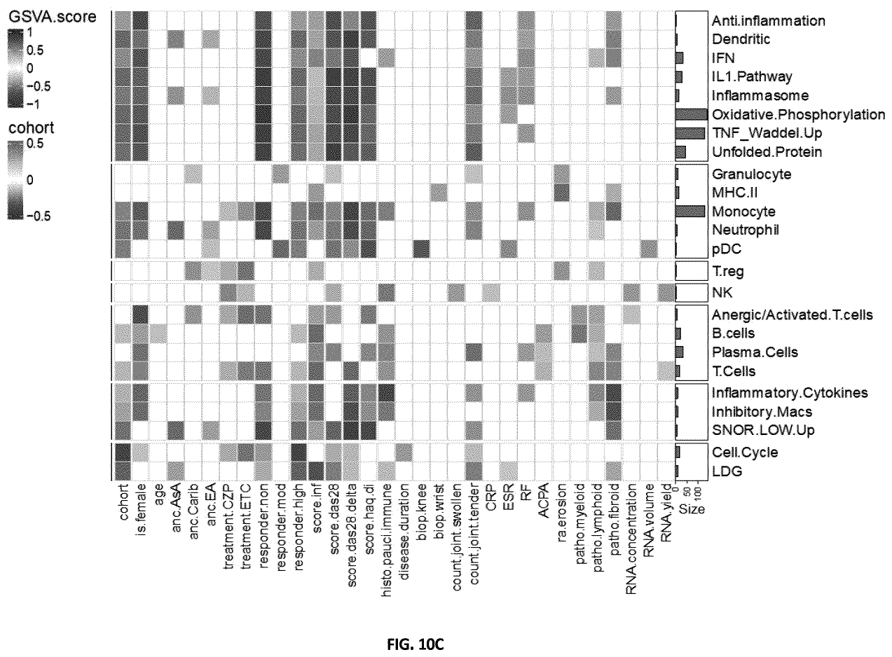

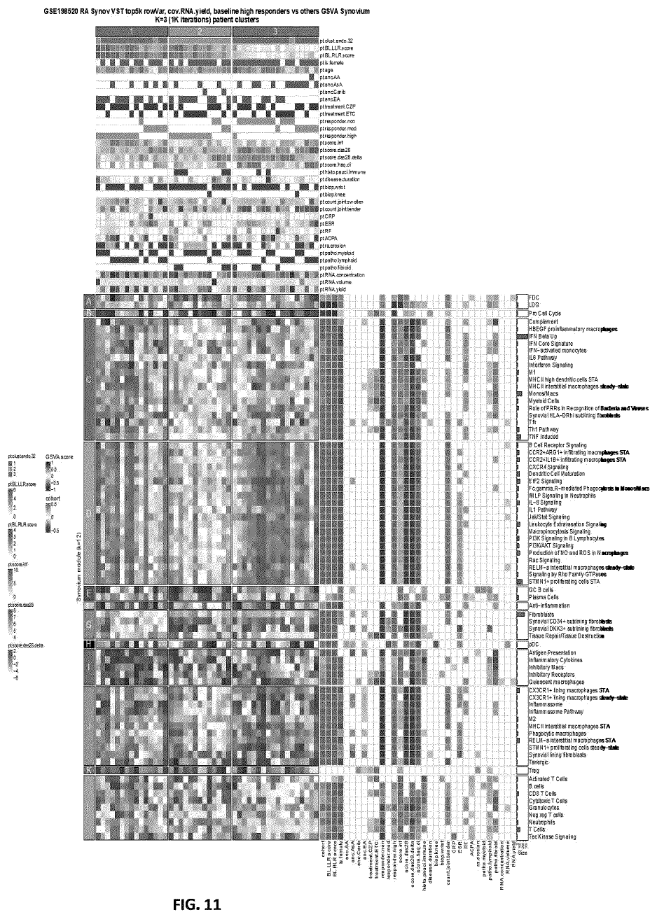

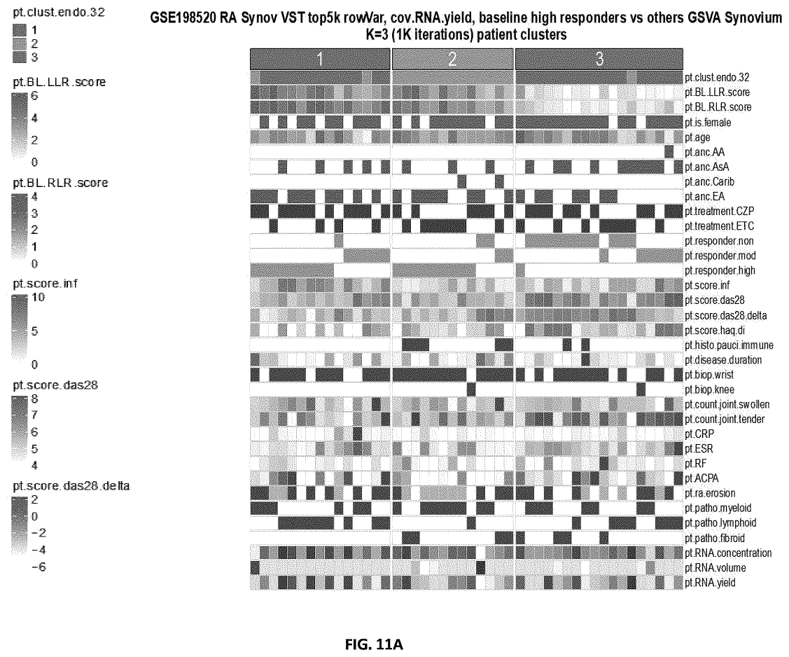

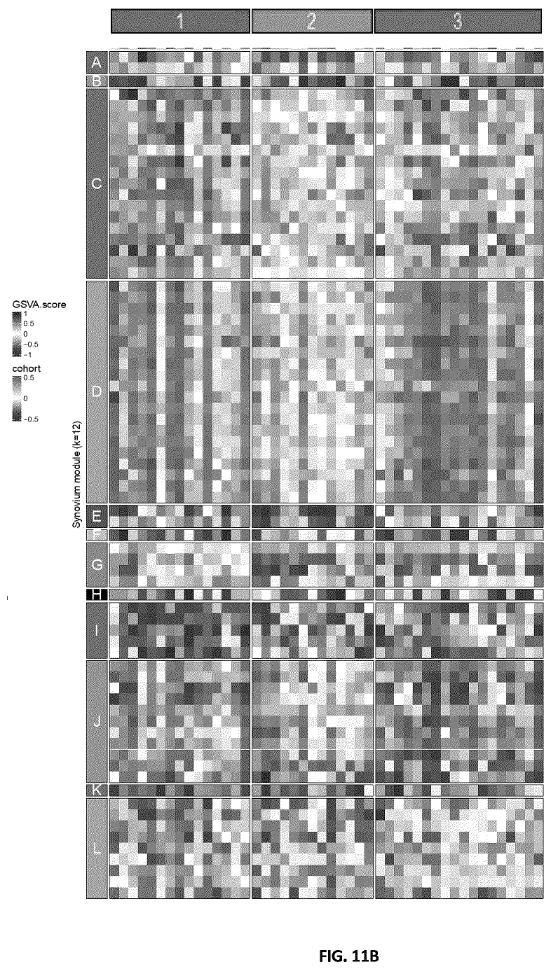

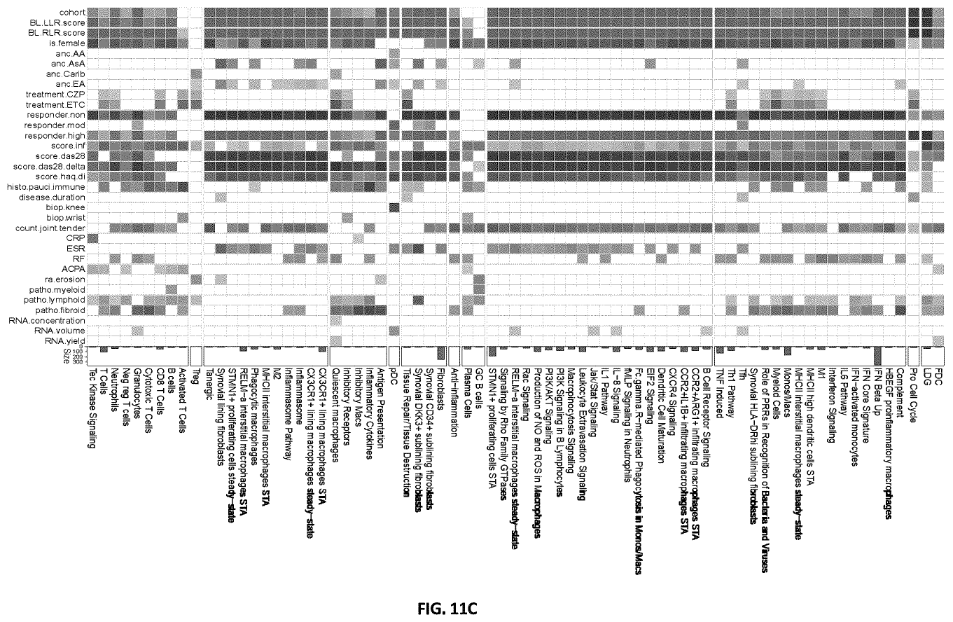

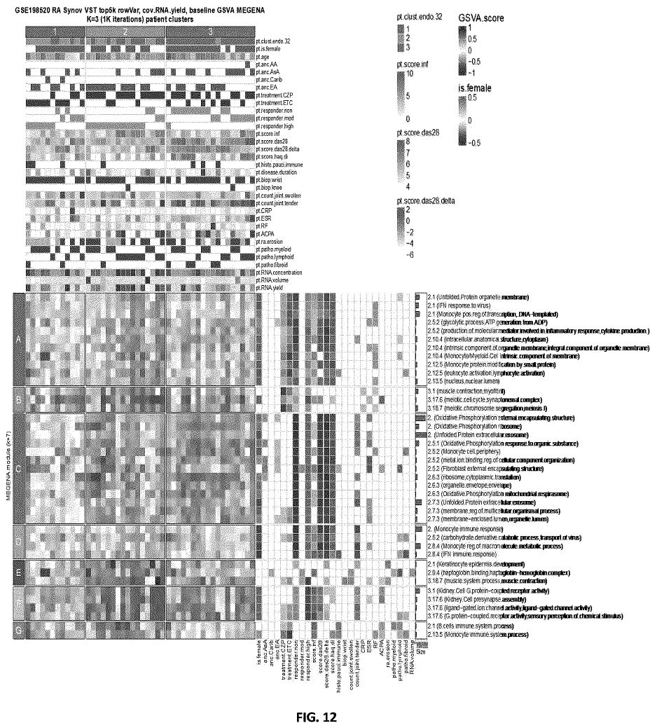

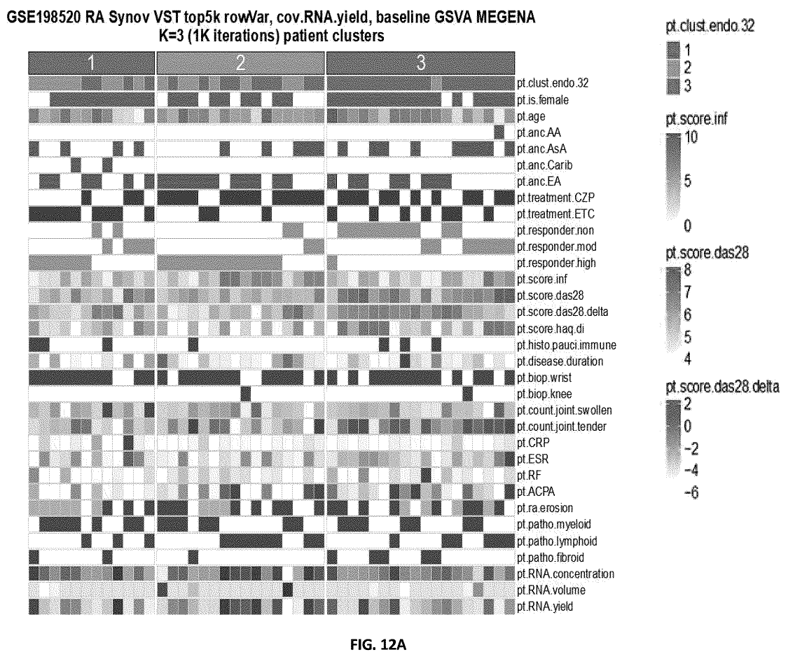

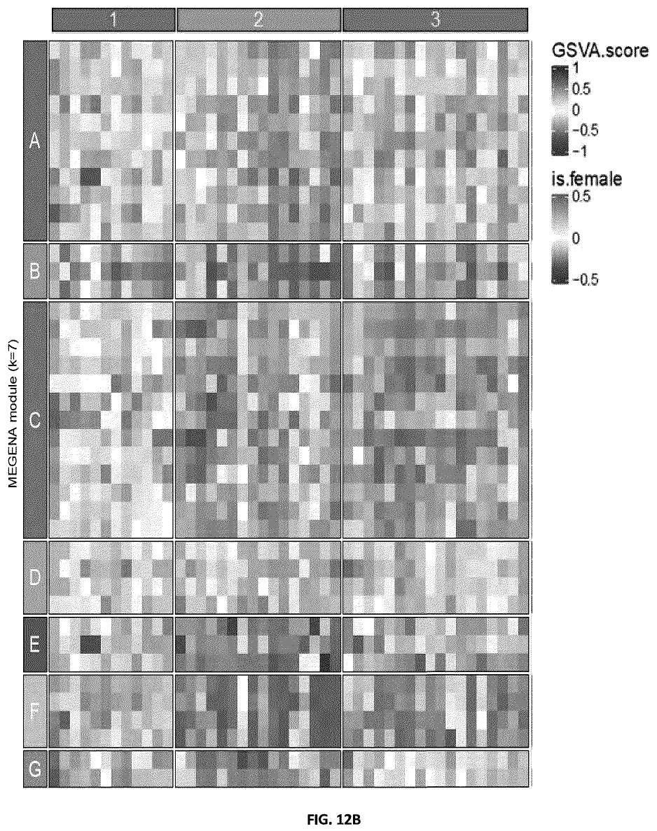

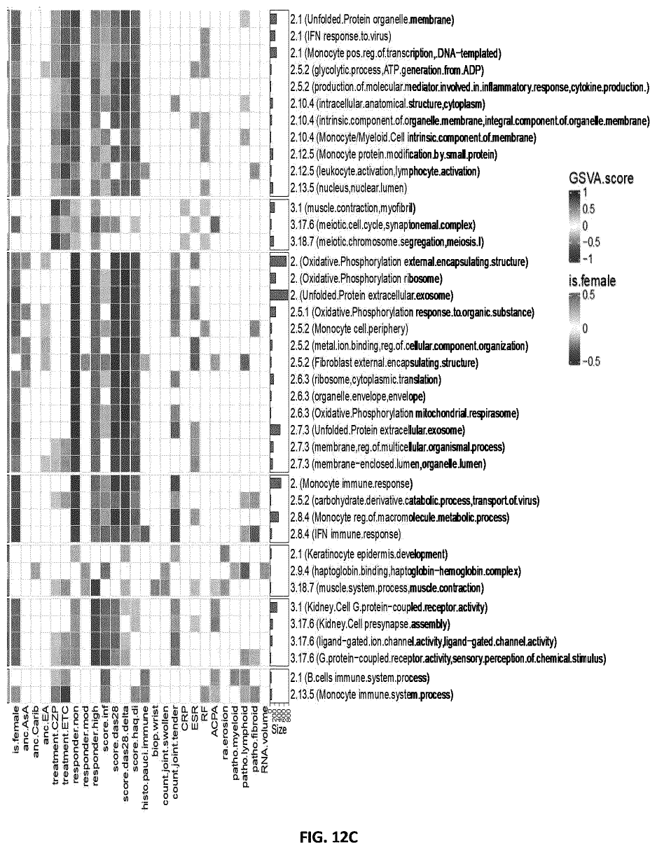

In some embodiments, the gene modules described herein comprise genes associated with a disease or disorder as described herein. In some embodiments, the gene modules described herein comprise genes associated with a disease or disorder as shown in FIG. 7, FIG. 8, FIG. 9, FIG. 10, FIG. 11, or FIG. 12. In some embodiments, the gene modules described herein are associated with a disease or disorder, treatment, process, or cell type as shown in FIG. 7, FIG. 8, FIG. 9, FIG. 10, FIG. 11, or FIG. 12. In some embodiments, a disease or disorder is associated with one or more gene modules as described herein. In some embodiments, a molecular endotype is associated with one or more gene modules as described herein. In some embodiments, a biological pathway is associated with one or more gene modules as described herein. In some embodiments, a phenotype is associated with one or more gene modules as described herein. In some embodiments, one or more gene modules as described herein are associated with the disease or disorder, the progression of a disease or disorder, the deterioration, the disability, the organ involvement, the medication response, the treatment response, the treatment target, the molecular endotype, or any combination thereof.

In some embodiments, the gene modules as described herein are significant gene modules. In some embodiments, the significant gene modules comprise a minimum number of genes. In some embodiments, the minimum number of genes as described herein is considered a threshold minimum size. In some embodiments, the threshold minimum size is about 2 genes to about 50 genes. In some embodiments, a plurality of gene modules comprise the gene modules described herein. In some embodiments, the plurality of gene modules comprise one or more gene modules described herein. In some embodiments, the plurality of gene modules comprise 2 or more gene modules. In some embodiments, the plurality of gene modules comprise 5 or more gene modules. In some embodiments, the plurality of gene modules comprise 10 or more gene modules. In some embodiments, the plurality of gene modules comprise 15 or more gene modules. In some embodiments, the plurality of gene modules comprise 20 or more gene modules. In some embodiments, the plurality of gene modules comprise 25 or more gene modules. In some embodiments, the plurality of gene modules comprise 30 or more gene modules. In some embodiments, the plurality of gene modules comprise 35 or more gene modules. In some embodiments, the plurality of gene modules comprise 40 or more gene modules. In some embodiments, the plurality of gene modules comprise 45 or more gene modules. In some embodiments, the plurality of gene modules comprise 50 or more gene modules. In some embodiments, the plurality of gene modules comprise 55 or more gene modules. In some embodiments, the plurality of gene modules comprise 60 or more gene modules. In some embodiments, the plurality of gene modules comprise 65 or more gene modules. In some embodiments, the plurality of gene modules comprise 70 or more gene modules. In some embodiments, the plurality of gene modules comprise 75 or more gene modules. In some embodiments, the plurality of gene modules comprise 80 or more gene modules.

In some embodiments, the plurality of gene modules comprises at least 2 to at least 80 gene modules. In some embodiments, the plurality of gene modules comprises at least 5 to at least 80 gene modules. In some embodiments, the plurality of gene modules comprises at least 10 to at least 80 gene modules. In some embodiments, wherein the plurality of gene modules comprises gene modules that are most strongly correlated with the one or more sample traits as described herein. In some embodiments, at least 2 to at least 80 gene modules are most strongly correlated with the one or more sample traits as described herein. In some embodiments, at least 5 to at least 80 gene modules are most strongly correlated with the one or more sample traits as described herein. In some embodiments, at least 10 to at least 80 gene modules are most strongly correlated with the one or more sample traits as described herein.

In some embodiments, the methods described herein comprise gene modules comprising genes as described herein. In some embodiments, the gene modules comprise genes of the gene set. In some embodiments, the gene modules are significant gene modules as described herein. In some embodiments, the gene modules described herein comprise genes, as shown in TABLE 2, TABLE 3, TABLE 4, or TABLE 5. In some embodiments, the significant gene modules described herein comprise genes, as shown in TABLE 2, TABLE 3, TABLE 4, or TABLE 5.

In some embodiments, the genes listed within each Table form a significant gene module. In some embodiments, genes listed within different Tables form different gene modules. In some embodiments, the genes listed within TABLE 2, TABLE 3, TABLE 4, or TABLE 5 form a gene module. In some embodiments, the genes listed within TABLE 2, TABLE 3, TABLE 4, or TABLE 5 form a significant gene module. In some embodiments, the genes listed within a different Table (e.g., TABLE 2, TABLE 3, TABLE 4, TABLE 5) form a different gene module. In some embodiments, the genes listed within a different Table (e.g., TABLE 2, TABLE 3, TABLE 4, TABLE 5) form a different significant gene module. In some embodiments, the genes listed in each of one or more Tables (e.g., TABLE 2, TABLE 3, TABLE 4, or TABLE 5) form the gene module. In some embodiments, the genes listed in each of one or more Tables (e.g., TABLE 2, TABLE 3, TABLE 4, or TABLE 5) form the significant gene module. In some embodiments, the genes listed within a Table (e.g., TABLE 2, TABLE 3, TABLE 4, or TABLE 5) form a significant gene module, and the genes listed within a different Table (e.g., TABLE 2, TABLE 3, TABLE 4, or TABLE 5) form a different significant gene module.

### I. Methods

Provided herein are methods for disease prediction and uses thereof. In general, methods described herein may be useful in developing targeted personalized treatment for patients with chronic, inflammatory, and/or autoimmune diseases. In some embodiments, methods for treating, preventing, or inhibiting a disease or disorder in a patient comprise identifying subsets of patients or different disease phenotypes as related to gene expression data. In some embodiments, methods for treating, preventing, or inhibiting a disease or disorder in a patient comprise disease prediction of different subsets of patients as related to gene expression data. In some embodiments, methods for predicting the clinical outcome of a patient comprise identifying subsets of patients based on gene expression data. In some embodiments, methods described herein comprise complex data analysis for disease prediction. In some embodiments, disease prediction involves a machine learning model, algorithm, and/or classifier. In some embodiments, disease prediction involves a machine learning model. In some embodiments, disease prediction involves a machine learning algorithm. In some embodiments, disease prediction involves a machine learning classifier. In some embodiments, methods described herein comprise analysis of gene expression data generated from assaying a sample from a patient. In some embodiments, methods describe herein comprise analyzing gene expression data from one or more genes to determine coexpression patterns. In some embodiments, methods described herein comprise analyzing gene expression data from one or more genes to determine one or more genes with similar gene expression patterns. In some embodiments, methods described herein comprise analyzing gene expression data from one or more genes to determine a molecular endotype. In some embodiments, methods described herein comprise analyzing gene expression data to determine a gene set. In some embodiments, methods described herein comprise analyzing gene expression data to determine a gene module. In some embodiments, methods described herein comprise a gene set associated to a disease or disorder. In some embodiments, methods described herein comprise a gene module associated to a disease or disorder.

### II. Methods for Determining a Gene Set

Provided herein are methods for determining a gene set or gene module capable of classifying a disease or disorder of a patient. In some embodiments, analysis of gene expression data results in the categorization of genes as gene sets or gene modules. In some embodiments, analysis of gene expression data results in the identification of similar gene expression profiles. In some embodiments, gene expression profiles are patterns of gene expression identified from gene expression data. In some embodiments, analysis of gene expression data for one or more genes results in the identification of similar expression profiles for one or more genes. In some embodiments, genes with similar expression profiles are categorized into gene sets. In some embodiments, one or more genes with similar expression profiles are categorized into gene sets. In some embodiments, one or more genes with similar expression profiles are categorized into gene modules. In some embodiments, the gene expression profiles are associated with a disease or disorder. In some embodiments, the gene expression data is associated with a disease or disorder. In some embodiments, the gene modules are associated with a disease or disorder. In some embodiments, the gene modules classify the disease or disorder in a patient. In some embodiments, the gene expression data generated from assaying a biological sample from a patient is compared to gene modules. In some embodiments, the gene expression data generated from assaying a biological sample from a patient identifies the disease or disorder in a patient. In some embodiments, the gene expression data generated from assaying a biological sample from a patient is compared to the gene modules, thus identifying the presence or absence of a disease or disorder in a patient. In some embodiments, the gene expression data generated from assaying a biological sample from a patient is compared to gene modules, thus identifying the immunological state of a patient, the susceptibility of a patient to a disease or disorder, the progression of a disease or disorder of a patient, the rate of deterioration of a patient as related to a disease or disorder, the likelihood that a patient will respond to a particular treatment for a disease or disorder, or combinations thereof. In some embodiments, the gene expression data generated from assaying a biological sample from a patient is compared to gene modules, thus predicting a clinical outcome of the disease or disorder of the patient. In some embodiments, the gene expression data generated from assaying a biological sample from a patient is used for the identification of a treatment, treatment schedule, or drug that correlates to the best clinical outcome for the patient. In some embodiments, the gene expression data generated from assaying the biological sample from the patient is used to identify different subsets of patients corresponding to a different disease phenotype. In some embodiments, the gene expression data generated from assaying the biological sample from the patient is used to identify different subsets of patients corresponding to a different clinical outcome. In some embodiments, the gene expression data generated from assaying the biological sample from the patient is used to identify different subsets of patients corresponding to a different treatment. In some embodiments, the gene expression data generated from assaying the biological sample from the patient is used to identify different clusters of patients corresponding to a different treatment. In some embodiments, the gene expression data generated from assaying the biological sample from the patient is used to identify different subsets of patients corresponding to a different disease phenotype, with the different disease phenotype requiring a different treatment. In some embodiments, the gene expression data generated from assaying the biological sample from the patient is used to identify different subsets of patients corresponding to a different molecular endotype, with the different molecular endotype requiring a different treatment. In some embodiments, the phenotype is associated with a disease or disorder, a progression of a disease or disorder, a trajectory of a disease or disorder, a deterioration, a disability, an organ involvement, a medication response, a treatment response, a treatment target, a treatment, a treatment recommendation, a molecular endotype, or combinations thereof. In some embodiments, the trajectory of the disease or disorder comprises patient decline comprising slow decline, gradual decline, stair step decline, or rapid decline.

In some embodiments, the methods described herein are methods for generating a data set comprising gene expression data by assaying a biological sample from a patient. In some embodiments, the methods described herein comprise generating gene expression data by measuring the concentration of a nucleic acid in a biological sample. In some embodiments, the methods described herein comprise generating gene expression data by analyzing the transcriptome in a biological sample. In some embodiments, the methods described herein comprise generating gene expression data by sequencing the nucleic acid in a biological sample. In some embodiments, the methods described herein comprise assaying a biological sample from a patient. In some embodiments, the methods described herein result in the identification of a molecular endotype. In some embodiments, the gene expression data from a biological sample from a patient results in the identification of the molecular endotype of the patient. In some embodiments, the molecular endotype is indicative of a subtype of a disease or disorder. In some embodiments, the molecular endotype is indicative of the cellular pathways related to a disease or disorder. In some embodiments, the molecular endotype is indicative of the disease susceptibility of the patient to a disease or disorder. In some embodiments, the molecular endotype is indicative of the treatment with the highest probability of success for a patient. In some embodiments, the treatment with the highest probability of success relates to a treatment that is the most likely to prevent a disease or disorder, reduce the rate of deterioration related to a disease or disorder, reduce or stop the progression of a disease or disorder, reduce or stop the symptoms of the disease or disorder, increase the rate of regeneration of the patient as related to the disease or disorder, or combinations thereof. In some embodiments, the treatment that has the highest probability of success is the most effective treatment.

In some embodiments, assaying a biological sample from a patient as described herein comprises measuring the concentration of a nucleic acid in a biological sample, analyzing the transcriptome in a biological sample, sequencing a nucleic acid in a biological sample, or combinations thereof. In some embodiments, assaying a biological sample from a patient as described herein comprises the use of any method for generating gene expression data. In some embodiments, assaying a biological sample from a patient as described herein comprises the use of any method for generating gene expression data that results in the identification of a molecular endotype. In some embodiments, assaying a biological sample from a patient as described herein comprises the use of sequencing, next-generation sequencing (NGS), RNA sequencing (RNA-seq), microarrays, DNA microarrays, RNA microarrays, quantitative PCR (qPCR), or combinations thereof.

Also provided herein are methods for determining a gene set capable of classifying a disease or disorder. In some embodiments, the method for determining a gene set capable of classifying a disease or disorder comprise N genes moduled into gene modules as described herein. In some embodiments, the N genes are selected from the initial gene set or the first gene set based on variation in the gene expression within the plurality of reference biological samples.

In some embodiments, analyzing a data set to select N genes from an initial gene set, where Nis an integer number. The data set can comprise gene expression data of genes of the initial gene set, from a plurality of reference biological samples. The plurality of reference biological samples can be obtained or derived from a plurality of reference patients. In some embodiments, analyzing the dataset can comprise obtaining a first gene set from the initial gene set, and selecting the N genes from the first gene set. The first gene set can be a subset of the initial gene set. Each genes of the first gene set can be mapped to at least one known protein. The first gene set can be obtained from the initial gene set by removing genes that cannot be mapped to a known protein. In some embodiments, the genes within the first gene set are protein coding genes. In some embodiments, the mapping is performed using the publicly available R BioMaRt package to query probes for any corresponding HGNC gene symbol mappings. The N genes can be selected from the initial gene set or the first gene set based on variation in the gene expression within the plurality of reference biological samples. In some embodiments, the N genes are N variably expressed genes of the initial gene set or the first gene set or both. In some embodiments, the N genes are N variably expressed genes of the initial gene set. In some embodiments, the N genes are N variably expressed genes of the first gene set. In some embodiments, the N genes are N most variably expressed genes of the initial gene set or the first gene set or both. In some embodiments, the N genes are N most variably expressed genes of the initial gene set. In some embodiments, the N genes are N most variably expressed genes of the first gene set. The variable expression can be based on gene expression in the plurality of reference biological samples. In some embodiments, the genes selected from the initial gene set and/or the first gene set comprise the N genes. In some embodiments, the genes selected from the initial gene set and/or the first gene set comprise the N genes and an additional gene. In some embodiments, the genes selected from the initial gene set and/or the first gene set comprise the N genes and no additional gene. In some embodiments, the N genes are grouped into a gene module. In some embodiments, the N genes within a gene module correlate to a gene expression profile. In some embodiments, the N genes are grouped into a plurality of gene modules. In some embodiments, one or more gene modules of the plurality of gene modules are correlated with one or more sample traits. In some embodiments, comprise one or more gene modules of the plurality of gene modules are correlated with one or more sample traits of the plurality of reference patients. In some embodiments, a plurality of significant gene modules are selected from the plurality of gene modules correlated with one or more sample traits. comprise In some embodiments, the plurality of significant gene modules are selected from the plurality of gene modules based on strength of the correlation with one or more sample traits as described herein. In some embodiments, the genes within the plurality of significant gene modules form the gene set capable of classifying the disease or disorder of the patient. In some embodiments, the gene set capable of classifying the disease or disorder of the patient comprises a characteristic gene expression profile. In some embodiments the gene set capable of classifying the disease or disorder of the patient is correlated with sample traits. In some embodiments, the gene expression data set generated from assaying an isolated biological sample comprises gene expression data from the genes within the gene modules. In some embodiments, the gene expression data set generated from assaying the isolated biological sample comprises gene expression data from the genes within the plurality of gene modules. In some embodiments, the gene expression data set generated from assaying the isolated biological sample comprises gene expression data from the genes within the plurality of significant gene modules. In some embodiments, the gene expression data is capable of classifying the disease or disorder of the patient. In some embodiments, the gene expression data of the gene set is capable of classifying the disease or disorder of the patient. In some embodiments, the gene expression data of the genes of the gene set is capable of classifying the disease or disorder of the patient.

In some embodiments, the plurality of gene modules are correlated with one or more sample traits, selecting a plurality of significant gene modules based at least on strength of the correlation, overlapping one or more significant gene modules with one or more gene function signature lists, annotating the one or more significant gene modules with one or more functional characterizations based on sufficient overlap between one or more significant gene modules and the one or more gene function signature lists such that sufficient overlap satisfies overlap of a threshold minimum number of genes, and patients with a disease or disorder are determined to correspond to different patient populations or different treatment populations. In some embodiments, patients in different patient populations receive different treatments for the disease or disorder. In some embodiments, sufficient overlap between one or more significant gene modules and a functional characterization group satisfies overlap of a threshold minimum number of genes between one or more significant gene modules and a functional characterization group. In some embodiments, the threshold minimum number of genes are about 3 genes to about 12 genes. In some embodiments, the overlap is measured by any suitable technique. In some embodiments, the overlap is measured using fisher's exact test. In some embodiments, the sufficient overlap (e.g., for the threshold minimum number of genes) has a threshold Fisher's adjusted p value. In some embodiments, the threshold Fisher's adjusted p value for sufficient overlap can be about <0.3, about <0.2, or <0.1.

In some embodiments, the one or more gene function signature lists are selected from the one or more gene function signature lists in TABLE 7.

In some embodiments, the gene function signature lists, the functional characterization groups (e.g., categories) within the list, and genes within the functional characterization groups for AMPEL Endotype.32 (Endo.32), AMPEL Ancestry (Anc), AMPEL tissues (Tis), and Biologically Informed Gene Clustering (BIG-C), are provided in Catalina, Michelle D., et al. “Patient ancestry significantly contributes to molecular heterogeneity of systemic lupus erythematosus.” JCI insight 5.15 (2020); for GO is publicly available at http://geneontology.org/; for BRETIGEA is provided in Mckenzie, Andrew T., et al. “Brain cell type specific gene expression and coexpression network architectures.” Scientific reports 8.1 (2018): 1-19; for Hallmark gene sets, KEGG Pathway Database, Reactome signature is publicly available at http://www.gsea-msigdb.org/gsea/msigdb/collections.jsp.

In some embodiments, classifying the disease or disorder of the patient comprises determining whether the patient has a disease or disorder. In some embodiments, classifying the disease or disorder of the patient comprises determining a molecular endotype. In some embodiments, classifying the disease or disorder of the patient comprises determining the patient's molecular endotype. As described herein, the terms “molecular endotype” and “endotype” may be used interchangeably. In some embodiments, classifying the disease or disorder of the patient comprises determining the patient's molecular endotype out of a group of endotypes. In some embodiments, classifying the disease or disorder of the patient comprises determining the patient's molecular endotype out of two or more endotypes. In some embodiments, classifying the disease or disorder of the patient comprises determining the group of endotypes associated with the gene expression data. In some embodiments, classifying the disease or disorder of the patient comprises determining the group of endotypes associated with the disease or disorder. In some embodiments, classifying the disease or disorder of the patient comprises determining the endotypes associated with the gene set. In some embodiments, classifying the disease or disorder of the patient comprises determining the disease endotype distribution within a patient population comprising reference patients. some comprise

In some embodiments, classifying the disease or disorder of the patient is associated with the disease endotype distribution within reference patients.

In some embodiments, the patient population comprising reference patients comprises two or more endotypes. In some embodiments, the patient population comprises a plurality of reference patients.

In some embodiments, the plurality of reference patients comprises a first plurality of reference patients having a first endotype of the disease or disorder, a second plurality of reference patients having a second endotype of the disease or disorder, and a third plurality of reference patients having a third endotype of the disease or disorder. In some embodiments, classifying the disease or disorder of the patient comprises classifying whether the patient has the first endotype of the disease or disorder, the second endotype of the disease or disorder, or the third endotype of the disease or disorder.

In some embodiments, the method optionally comprises functionally annotating the plurality of significant gene modules. In some embodiments, the method can optionally comprise functionally annotating the plurality of gene modules. In some embodiments, the group of endotypes comprises all endotypes of the disease or disorder. In some embodiments, the group of endotypes does not comprise all endotypes of the disease or disorder. In some embodiments, the two or more endotypes comprise all endotypes of the disease or disorder. In some embodiments, the two or more endotypes do not comprise all endotypes of the disease or disorder comprise. In some embodiments, the reference patients comprise healthy controls. In some embodiments, the reference patients do not comprise healthy controls. In some embodiments, the comprise methods described herein comprise the use of a computer. In some embodiments, the methods described herein comprise analyzing data with a computer. In some embodiments, the methods described herein are performed with the use of a computer. In some embodiments, the methods described herein are implemented with the use of a computer.

In some embodiments, the data set comprises a plurality of individual data sets. In some embodiments, the plurality of individual data sets is obtained from the plurality of reference patients. In some embodiments, a data set is obtained from a refence patient. In some embodiments, an individual data set is obtained from a reference patient. In some embodiments, a data set is obtained from each reference patient. In some embodiments, an individual data set of a plurality of individual data sets is obtained from each reference patient of a plurality of reference patients. In some embodiments, different individual data sets are obtained from different reference patients. In some embodiments, an individual data set comprises gene expression data. In some embodiments, an individual data set comprises gene expression data from a reference biological sample. In some embodiments, an individual data set comprises gene expression data from a reference biological sample from a reference patient. In some embodiments, an individual data set comprises reference gene expression data. In some embodiments, a data set comprises reference gene expression data. comprise In some embodiments, the data set comprises gene expression data from the genes of the initial gene set. In some embodiments, each individual data set comprises gene expression data from a reference biological sample from a reference patient of the plurality of reference patients, of the genes of the initial gene set.

In some embodiments, the genes of the initial gene set are genes, protein coding genes, transcribed genes, or subsets thereof. In some embodiments, the genes of the initial gene set are genes, protein coding genes, transcribed genes, or subsets thereof, in the plurality of reference biological samples. In some embodiments, genes in the initial gene set are genes, protein coding genes, transcribed genes, or subsets thereof, for which gene expression data from the plurality of reference biological samples is available in the data set. In some embodiments, genes in the initial gene set are genes, protein coding genes, transcribed genes, or subsets thereof, for which gene expression data from each reference biological sample of the plurality of reference biological samples is available in the data set. In some embodiments, the subsets of genes, protein coding genes, or transcribed genes are obtained by removing genes, protein coding genes, or transcribed genes, respectively. In some embodiments, removing genes as described herein comprises removing genes with low copy number. In some embodiments, removing genes as described herein comprises removing genes that one of skill in the art would want to remove from the subset of genes.

In some embodiments, the N genes are N most variably expressed genes of the initial gene set in the data set. In some embodiments, the N genes are N most variably expressed genes of the first gene set in the data set. In some embodiments, the N genes are N most variably expressed genes of the initial gene set and/or the first gene set in the data set. In some embodiments, N most variably expressed genes are selected and grouped. In some embodiments, selected and grouped N most variably expressed genes are useful for dimensionality reduction, obtaining high quality data for gene grouping and subsequent analysis, reducing noise from the data, improving speed of computer systems, or combinations thereof.

In some embodiments, the N most variably expressed genes are selected using variable expression. In some embodiments, variable expression is measured using row variance. In some embodiments, genes with higher variable expression within the plurality of reference biological samples have higher row variance. In some embodiments, average row variance is calculated. In some embodiments, average row variance is stored as a matrix. In some embodiments, the matrix comprises the averaged gene expressions of each gene. In some embodiments, the matrix comprises the averaged gene expressions of each gene (e.g., initial gene set or the first gene set) as rows. In some embodiments, the matrix comprises the averaged samples (e.g., reference patients/reference biological samples) as columns. In some embodiments, the matrix comprises the averaged gene expressions of each gene as rows, and samples as columns. In some embodiments, the matrix is sorted by decreasing average row variance. In some embodiments, the matrix is sorted and the top N genes are selected to obtain N most variably expressed genes. In some embodiments, the matrix is sorted by decreasing average row variance and the top N genes can be selected, to obtain N most variably expressed genes. In some embodiments, using row variance allows obtaining modules in an unsupervised and statistically non-biased manner. In some embodiments, using row variance allows obtaining modules in an unsupervised and statistically non-biased manner based on statistically significant gene expression data. In some embodiments, the method comprises data sets comprising healthy controls. In some embodiments, the method comprising data sets comprising no healthy controls.

In some embodiments, N is about 500 to about 10,000. In some embodiments, N is about 500 to about 10,000, most variably expressed genes of the initial gene set or the first gene set or both. In some embodiments, N is about 500 to about 1,000, 500 to about 2,000, about 500 to about 3,000, about 500 to about 4,000, about 500 to about 4,500, about 500 to about 5,000, about 500 to about 5,500, about 500 to about 6,000, about 500 to about 7,000, about 500 to about 8,000, about 500 to about 9,000, about 500 to about 10,000, 1,000 to about 2,000, about 1,000 to about 3,000, about 1,000 to about 4,000, about 1,000 to about 4,500, about 1,000 to about 5,000, about 1,000 to about 5,500, about 1,000 to about 6,000, about 1,000 to about 7,000, about 1,000 to about 8,000, about 1,000 to about 9,000, about 1,000 to about 10,000, about 2,000 to about 3,000, about 2,000 to about 4,000, about 2,000 to about 4,500, about 2,000 to about 5,000, about 2,000 to about 5,500, about 2,000 to about 6,000, about 2,000 to about 7,000, about 2,000 to about 8,000, about 2,000 to about 9,000, about 2,000 to about 10,000, about 3,000 to about 4,000, about 3,000 to about 4,500, about 3,000 to about 5,000, about 3,000 to about 5,500, about 3,000 to about 6,000, about 3,000 to about 7,000, about 3,000 to about 8,000, about 3,000 to about 9,000, about 3,000 to about 10,000, about 4,000 to about 4,500, about 4,000 to about 5,000, about 4,000 to about 5,500, about 4,000 to about 6,000, about 4,000 to about 7,000, about 4,000 to about 8,000, about 4,000 to about 9,000, about 4,000 to about 10,000, about 4,500 to about 5,000, about 4,500 to about 5,500, about 4,500 to about 6,000, about 4,500 to about 7,000, about 4,500 to about 8,000, about 4,500 to about 9,000, about 4,500 to about 10,000, about 5,000 to about 5,500, about 5,000 to about 6,000, about 5,000 to about 7,000, about 5,000 to about 8,000, about 5,000 to about 9,000, about 5,000 to about 10,000, about 5,500 to about 6,000, about 5,500 to about 7,000, about 5,500 to about 8,000, about 5,500 to about 9,000, about 5,500 to about 10,000, about 6,000 to about 7,000, about 6,000 to about 8,000, about 6,000 to about 9,000, about 6,000 to about 10,000, about 7,000 to about 8,000, about 7,000 to about 9,000, about 7,000 to about 10,000, about 8,000 to about 9,000, about 8,000 to about 10,000, or about 9,000 to about 10,000. In some embodiments, N is about 500 to about 1,000, 500 to about 2,000, about 500 to about 3,000, about 500 to about 4,000, about 500 to about 4,500, about 500 to about 5,000, about 500 to about 5,500, about 500 to about 6,000, about 500 to about 7,000, about 500 to about 8,000, about 500 to about 9,000, about 500 to about 10,000, 1,000 to about 2,000, about 1,000 to about 3,000, about 1,000 to about 4,000, about 1,000 to about 4,500, about 1,000 to about 5,000, about 1,000 to about 5,500, about 1,000 to about 6,000, about 1,000 to about 7,000, about 1,000 to about 8,000, about 1,000 to about 9,000, about 1,000 to about 10,000, about 2,000 to about 3,000, about 2,000 to about 4,000, about 2,000 to about 4,500, about 2,000 to about 5,000, about 2,000 to about 5,500, about 2,000 to about 6,000, about 2,000 to about 7,000, about 2,000 to about 8,000, about 2,000 to about 9,000, about 2,000 to about 10,000, about 3,000 to about 4,000, about 3,000 to about 4,500, about 3,000 to about 5,000, about 3,000 to about 5,500, about 3,000 to about 6,000, about 3,000 to about 7,000, about 3,000 to about 8,000, about 3,000 to about 9,000, about 3,000 to about 10,000, about 4,000 to about 4,500, about 4,000 to about 5,000, about 4,000 to about 5,500, about 4,000 to about 6,000, about 4,000 to about 7,000, about 4,000 to about 8,000, about 4,000 to about 9,000, about 4,000 to about 10,000, about 4,500 to about 5,000, about 4,500 to about 5,500, about 4,500 to about 6,000, about 4,500 to about 7,000, about 4,500 to about 8,000, about 4,500 to about 9,000, about 4,500 to about 10,000, about 5,000 to about 5,500, about 5,000 to about 6,000, about 5,000 to about 7,000, about 5,000 to about 8,000, about 5,000 to about 9,000, about 5,000 to about 10,000, about 5,500 to about 6,000, about 5,500 to about 7,000, about 5,500 to about 8,000, about 5,500 to about 9,000, about 5,500 to about 10,000, about 6,000 to about 7,000, about 6,000 to about 8,000, about 6,000 to about 9,000, about 6,000 to about 10,000, about 7,000 to about 8,000, about 7,000 to about 9,000, about 7,000 to about 10,000, about 8,000 to about 9,000, about 8,000 to about 10,000, or about 9,000 to about 10,000 most variably expressed genes of the initial gene set or the first gene set or both. In some embodiments, N is about 500, about 1,000, about 2,000, about 3,000, about 4,000, about 4,500, about 5,000, about 5,500, about 6,000, about 7,000, about 8,000, about 9,000, or about 10,000. In some embodiments, N is about 500, about 1,000, about 2,000, about 3,000, about 4,000, about 4,500, about 5,000, about 5,500, about 6,000, about 7,000, about 8,000, about 9,000, or about 10,000, most variably expressed genes of the initial gene set or the first gene set or both. In some embodiments, N is at most about 1,000, about 2,000, about 3,000, about 4,000, about 4,500, about 5,000, about 5,500, about 6,000, about 7,000, about 8,000, about 9,000, or about 10,000. In some embodiments, N is at most about 1,000, about 2,000, about 3,000, about 4,000, about 4,500, about 5,000, about 5,500, about 6,000, about 7,000, about 8,000, about 9,000, or about 10,000, most variably expressed genes of the initial gene set or the first gene set or both.

In some embodiments, the plurality of gene modules comprise N genes. In some embodiments, the gene module comprises N genes. In some embodiments, the plurality of gene modules comprise N genes, grouped in consideration of the coexpression of the N genes. In some embodiments, the coexpression of the N genes in a biological sample is assayed. In some embodiments, the coexpression of the N genes in a plurality of biological samples is assayed. In some embodiments, the co-expressed genes are grouped within a gene module. In some embodiments, genes with similar gene expression profiles are grouped within a gene module. In some embodiments, genes having similar expression profiles in the plurality of reference biological samples are grouped within a gene module. In some embodiments, coexpression of the N genes in the plurality of reference biological samples is analyzed using a gene coexpression network analysis. In some embodiments, the N genes are grouped into the plurality of gene modules based on the gene coexpression network analysis. In some embodiments, the gene coexpression network analysis is performed using multiscale embedded gene coexpression network analysis (MEGENA), and/or weighted gene coexpression network analysis (WGCNA). In some embodiments, the N genes are grouped into the plurality of gene modules using MEGENA and/or WGCNA. In some embodiments, the N genes are grouped into the plurality of gene modules using MEGENA. In some embodiments, the N genes are grouped into the plurality of gene modules using WGCNA. In some embodiments, the methods described herein comprise MEGENA as described herein. In some embodiments, the methods described herein comprise WGCNA as described herein. MEGENA, and/or WGCNA are performed using steps/methods as described herein, in the Examples, and/or as understood by one of skill in the art. In some embodiments, the N genes are grouped into the plurality of gene modules by developing a planar filtered network (PFN) graph based on gene pair coexpression of the N genes in the plurality of reference biological samples. In some embodiments, the N genes are grouped into the plurality of gene modules by developing a PFN graph based on gene pair coexpression of the N genes in the plurality of reference biological samples, and extracting multiscale modules existing within the PFN graph to form the plurality of gene modules. In some embodiments, the N genes are compared to each other to identify coexpression similarities. In some embodiments, two N genes coexpression with similar expression are paired as co-expressed genes. In some embodiments, gene comparisons to identify pairwise coexpression similarities are assigned a global false discovery rate (FDR) calculation. In some embodiments, gene pairs below a FDR p threshold are removed. In some embodiments, the FDR p threshold is <0.35, <0.3, <0.25, <0.2, <0.1, <0.05, or <0.01. In some embodiments, the FDR p threshold is <0.2. In some embodiments, the removal of gene pairs reduces the risk of a random choice affecting the strength of the correlation. In some embodiments, the gene pairs above the FDR p threshold are mapped onto a sunburst diagram. In some embodiments, the gene pairs above the FDR p threshold are mapped onto a sunburst diagram, with edges created between them. In some embodiments, the “edge” in a coexpression network is a line/connection created between two “nodes” (genes), indicating similarity of gene expression between the two genes/nodes. In some embodiments, an algorithm maps gene pairs onto a sunburst diagram and identifies whether there is a remaining gene pair that must be added to the map. In some embodiments, a gene pair with a similar gene expression to the most recently added pair is added to the sphere. In some embodiments, a gene pair with a similar gene expression to the most recently added pair is joined by a new edge. In some embodiments, the gene pair placement scheme as described herein continues throughout the sunburst diagram, such that edges do not cross each other. In some embodiments, edges do not cross each other (meaning there are no exactly similar coexpression placements) and the network can inherently grow to occupy the sunburst diagram's surface. In some embodiments, the gene pairs above the FDR p threshold are placed onto the sunburst diagram and edges are drawn between them. In some embodiments, “triangulated” edges are drawn between gene pair neighborhoods called “cliques”. In some embodiments, triangles of edges are formed between highly co-expressed gene pairs and gene pair neighborhoods or cliques. In some embodiments, triangles of edges are formed between highly co-expressed gene pairs and gene pair neighborhoods or cliques, are deemed gene modules, becoming the first “generation” of gene modules. The algorithm continues by searching for further triangulations within the first generation (gen1) of clique modules. Those genes that are most connected (given a compactness a parameter according to the algorithm) within cliques are inherited together as descendent modules (e.g., modules), whereas the disconnected genes are discarded and not inherited. This process continues as gen1 module undergo the scrutiny of the algorithm and give way to second generation (gen2) and subsequent generations until no further meaningful triangulations are found within the terminal descendants, and/or minimum threshold module size requirement is reached. In some embodiments, a “multi-scale” network of modules is created. This translates as modules of genes whose descendants become more and more densely connected by coexpression in the plurality of reference biological samples, with the implication the terminal descendants are most closely related by a shared biological regulatory network.

In some embodiments, the N genes are grouped into the plurality of gene modules by developing a planar filtered network (PFN) graph based on gene pair coexpression of the N genes, and extracting multiscale modules existing within the PFN graph to form the plurality of gene modules. In some embodiments, the PFN graph is generated by forming an adjacency matrix based on gene pair coexpression; ordering gene pairs according to strength of interaction and meeting a minimal false discovery rate; mapping gene pairs onto a sphere and add edges between them if and only if the resulting graph can still be embedded on a surface of a given genus g=k, where the edges are prohibited from crossing each other and the network wraps around on itself as the topological triangulations between cliques covering the sphere. In some embodiments, the extracting multiscale modules existing within the PFN graph comprises iteratively extracting multiscale modules from topological cliques, wherein the iteration continues until a threshold a resolution parameter is met, and the module sizes decrease and approach the minimum threshold module size requirement (e.g., a threshold minimum size). In some embodiments, a second pass of statistical stringency can be performed to eliminate modules not meeting desired module requirements including minimal and maximum module size and significant gene module compactness. In some embodiments, multiscale hub analysis (MHA) can be performed to identify module hub genes, defined as those genes with intramodular connections meeting a minimal significant hub degree.

In some embodiments, the one or more sample traits comprise clinical traits such as disease severity index, disease diagnostic parameter, etc.; of the reference patients. In some embodiments, the one or more sample traits comprise biographical traits such as age, ancestry, gender, etc.; of the reference patients. In some embodiments, the one or more sample traits comprise lifestyle traits such as some drug usage, smoking habits, drinking habits, exercise habits, etc.; of the reference patients. In some embodiments, the one or more sample traits comprise depend on the disease or disorder. In some embodiments, the one or more sample traits, depend on the endotype. In some embodiments, a sample trait of the one or more sample traits of the reference patient is a subjective sample trait or an objective sample trait. In some embodiments, the subjective sample trait comprises disease level (such as SLE Disease Activity Index (SLEDAI), a clinical index in the case of lupus), areas of pain, ancestry, gender, anecdotal features that are described by the patient or observed by a clinician but not objectively (quantifiably) measurable, or combinations thereof. In some embodiments, clinical considerations, objective laboratory assay results, and/or patient attributes for a subjective sample trait are retained as continuous numerical values, or encoded as discrete binary values (e.g., no=0 or yes=1). In some embodiments, the objective sample traits comprise blood autoimmune antibody level, blood complement component 3 (C3) protein level, age, drug usage, features that have quantifiable value, or combinations thereof.

In some embodiments, the strength of correlation of the gene modules with the sample traits as described herein is measured by a suitable method. In some embodiments, the strength of correlation of the gene modules with the one or more sample traits is measured by a suitable method. In some embodiments, the strength of correlation of the plurality of gene modules with the one or more sample traits is measured by a suitable method. In some embodiments, correlation and strength of correlation of the gene modules of the plurality of gene modules with the one or more sample traits is measured by a suitable method. In some embodiments, the one or more gene modules comprise all the gene modules of the plurality of gene modules. In some embodiments, all the gene modules of the plurality of gene modules are correlated with the one or more sample traits. In some embodiments, the one or more gene modules comprise a third generation gene module. In some embodiments, the one or more gene modules comprise a third generation gene modules of the plurality of gene modules. In some embodiments, the third generation gene modules of the plurality of gene modules are correlated with the one or more sample traits. In some embodiments, the plurality of gene modules are obtained using MEGENA. In some embodiments, the gene modules obtained using MEGENA are referred to as MEGENA modules. In some embodiments, third generation gene modules of the plurality of gene modules are correlated with the one or more sample traits, after the plurality of gene modules are obtained using MEGENA. In some embodiments, the third generation gene modules of the plurality of gene modules are MEGENA third generation gene modules. In some embodiments, the one or more gene modules comprise second, third and/or fourth generation gene modules. In some embodiments, the one or more gene modules comprise second, third and/or fourth generation gene modules of the plurality of gene modules. In some embodiments, the second, third and/or fourth generation gene modules are correlated with the one or more sample traits. In some embodiments, the second, third and/or fourth generation gene modules of the plurality of gene modules are correlated with the one or more sample traits, after the plurality of gene modules are obtained using MEGENA. In some embodiments, the second, third and/or fourth generation gene modules of the plurality of gene modules are MEGENA second, third and/or fourth generation gene modules, respectively. In some embodiments, the correlation of the one or more gene modules of the plurality of gene modules with one or more sample traits comprises correlating the module eigengenes (MEs). In some embodiments, the correlation of the one or more gene modules of the plurality of gene modules with one or more sample traits comprises correlating the MEs of the one or more gene modules with the one or more sample traits. In some embodiments, the correlation of the one or more gene modules with one or more sample traits comprises correlating the MEs of the one or more gene modules with the one or more sample traits, and selecting a plurality of significant gene modules based on the strength of correlation. In some embodiments, the correlation of the one or more gene modules with one or more sample traits comprises correlating the MEs of the one or more gene modules with one or more sample traits and selecting significant gene modules based on the strength of the correlation. In some embodiments, the MEs for each gene module is calculated. In some embodiments, the MEs for each gene module for each patient is calculated. In some embodiments, the MEs for each gene module for each reference patient is calculated. In some embodiments, the gene module MEs are correlated to one or more sample traits. In some embodiments, the gene module MEs are correlated to one or more sample traits of a reference patient. In some embodiments, the gene module MEs are correlated to one or more sample traits of a plurality of reference patients. In some embodiments, the plurality of reference patients and the gene modules are correlated, and the gene module MEs are correlated to one or more sample traits. In some embodiments, the gene module MEs of a patient are correlated to one or more sample traits of a patient. In some embodiments, wherein gene module MEs of a reference patient are correlated to one or more sample traits of the reference patient. In some embodiments, calculating the MEs of the gene module comprises gene expression data. In some embodiments, calculating the MEs of the gene module comprises a data set as described herein. In some embodiments, a group of genes is considered as a gene module for calculating the MEs of the gene module. In some embodiments, a group of genes with similar gene expression profiles is considered as a gene module for calculating the MEs of the gene module. In some embodiments, a plurality of reference patients is referred to as a cohort. In some embodiments, sample trait correlations for a plurality of reference patients or a cohort are assessed. In some embodiments, sample trait correlation that are not significant based on a threshold p value are set to zero. In some embodiments, for the plurality of reference patients, absolute value of significant correlation to cohort is ranked by row means, and gene modules with desired highest significant absolute value of mean correlations are selected as the plurality of significant gene modules. In some embodiments, for example, 30 gene modules are selected as the plurality of significant gene modules. In some embodiments, for example, the gene modules with the 30 highest significant absolute value of mean correlations are selected as the 30 gene modules. In some embodiments, the correlations are measured based on Pearson's correlation coefficient. In some embodiments, the threshold p value is 0.3, 0.25, 0.2, 0.1, 0.05, or 0.01. In some embodiments, the threshold p value is 0.2. In some embodiments, correlations with p values of <0.3, <0.25, <0.2, <0.1, <0.05, or <0.01, capture known and validated correlations and biological processes while maintaining statistical integrity and reproducibility.

In some embodiments, the plurality of significant gene modules comprise about 10 to about 80 gene modules. In some embodiments, the plurality of significant gene modules comprise about 10 gene modules to about 80 gene modules, that are most strongly correlated with the one or more sample traits. In some embodiments, the plurality of significant gene modules comprise about 10 gene modules to about 80 gene modules, that are correlated among the plurality of gene modules. In some embodiments, the plurality of significant gene modules comprise about 10 gene modules to about 80 gene modules that are correlated among the gene modules. In some embodiments, the plurality of significant gene modules comprise about 10 gene modules to about 80 gene modules that are most strongly correlated among the plurality of gene modules, with the one or more sample traits. In some embodiments, the plurality of significant gene modules comprise about 10 gene modules to about 20 gene modules, about 10 gene modules to about 25 gene modules, about 10 gene modules to about 30 gene modules, about 10 gene modules to about 35 gene modules, about 10 gene modules to about 40 gene modules, about 10 gene modules to about 45 gene modules, about 10 gene modules to about 50 gene modules, about 10 gene modules to about 55 gene modules, about 10 gene modules to about 60 gene modules, about 10 gene modules to about 70 gene modules, about 10 gene modules to about 80 gene modules, about 20 gene modules to about 25 gene modules, about 20 gene modules to about 30 gene modules, about 20 gene modules to about 35 gene modules, about 20 gene modules to about 40 gene modules, about 20 gene modules to about 45 gene modules, about 20 gene modules to about 50 gene modules, about 20 gene modules to about 55 gene modules, about 20 gene modules to about 60 gene modules, about 20 gene modules to about 70 gene modules, about 20 gene modules to about 80 gene modules, about 25 gene modules to about 30 gene modules, about 25 gene modules to about 35 gene modules, about 25 gene modules to about 40 gene modules, about 25 gene modules to about 45 gene modules, about 25 gene modules to about 50 gene modules, about 25 gene modules to about 55 gene modules, about 25 gene modules to about 60 gene modules, about 25 gene modules to about 70 gene modules, about 25 gene modules to about 80 gene modules, about 30 gene modules to about 35 gene modules, about 30 gene modules to about 40 gene modules, about 30 gene modules to about 45 gene modules, about 30 gene modules to about 50 gene modules, about 30 gene modules to about 55 gene modules, about 30 gene modules to about 60 gene modules, about 30 gene modules to about 70 gene modules, about 30 gene modules to about 80 gene modules, about 35 gene modules to about 40 gene modules, about 35 gene modules to about 45 gene modules, about 35 gene modules to about 50 gene modules, about 35 gene modules to about 55 gene modules, about 35 gene modules to about 60 gene modules, about 35 gene modules to about 70 gene modules, about 35 gene modules to about 80 gene modules, about 40 gene modules to about 45 gene modules, about 40 gene modules to about 50 gene modules, about 40 gene modules to about 55 gene modules, about 40 gene modules to about 60 gene modules, about 40 gene modules to about 70 gene modules, about 40 gene modules to about 80 gene modules, about 45 gene modules to about 50 gene modules, about 45 gene modules to about 55 gene modules, about 45 gene modules to about 60 gene modules, about 45 gene modules to about 70 gene modules, about 45 gene modules to about 80 gene modules, about 50 gene modules to about 55 gene modules, about 50 gene modules to about 60 gene modules, about 50 gene modules to about 70 gene modules, about 50 gene modules to about 80 gene modules, about 55 gene modules to about 60 gene modules, about 55 gene modules to about 70 gene modules, about 55 gene modules to about 80 gene modules, about 60 gene modules to about 70 gene modules, about 60 gene modules to about 80 gene modules, or about 70 gene modules to about 80 gene modules. In some embodiments, the plurality of significant gene modules comprise about 10 gene modules to about 20 gene modules, about 10 gene modules to about 25 gene modules, about 10 gene modules to about 30 gene modules, about 10 gene modules to about 35 gene modules, about 10 gene modules to about 40 gene modules, about 10 gene modules to about 45 gene modules, about 10 gene modules to about 50 gene modules, about 10 gene modules to about 55 gene modules, about 10 gene modules to about 60 gene modules, about 10 gene modules to about 70 gene modules, about 10 gene modules to about 80 gene modules, about 20 gene modules to about 25 gene modules, about 20 gene modules to about 30 gene modules, about 20 gene modules to about 35 gene modules, about 20 gene modules to about 40 gene modules, about 20 gene modules to about 45 gene modules, about 20 gene modules to about 50 gene modules, about 20 gene modules to about 55 gene modules, about 20 gene modules to about 60 gene modules, about 20 gene modules to about 70 gene modules, about 20 gene modules to about 80 gene modules, about 25 gene modules to about 30 gene modules, about 25 gene modules to about 35 gene modules, about 25 gene modules to about 40 gene modules, about 25 gene modules to about 45 gene modules, about 25 gene modules to about 50 gene modules, about 25 gene modules to about 55 gene modules, about 25 gene modules to about 60 gene modules, about 25 gene modules to about 70 gene modules, about 25 gene modules to about 80 gene modules, about 30 gene modules to about 35 gene modules, about 30 gene modules to about 40 gene modules, about 30 gene modules to about 45 gene modules, about 30 gene modules to about 50 gene modules, about 30 gene modules to about 55 gene modules, about 30 gene modules to about 60 gene modules, about 30 gene modules to about 70 gene modules, about 30 gene modules to about 80 gene modules, about 35 gene modules to about 40 gene modules, about 35 gene modules to about 45 gene modules, about 35 gene modules to about 50 gene modules, about 35 gene modules to about 55 gene modules, about 35 gene modules to about 60 gene modules, about 35 gene modules to about 70 gene modules, about 35 gene modules to about 80 gene modules, about 40 gene modules to about 45 gene modules, about 40 gene modules to about 50 gene modules, about 40 gene modules to about 55 gene modules, about 40 gene modules to about 60 gene modules, about 40 gene modules to about 70 gene modules, about 40 gene modules to about 80 gene modules, about 45 gene modules to about 50 gene modules, about 45 gene modules to about 55 gene modules, about 45 gene modules to about 60 gene modules, about 45 gene modules to about 70 gene modules, about 45 gene modules to about 80 gene modules, about 50 gene modules to about 55 gene modules, about 50 gene modules to about 60 gene modules, about 50 gene modules to about 70 gene modules, about 50 gene modules to about 80 gene modules, about 55 gene modules to about 60 gene modules, about 55 gene modules to about 70 gene modules, about 55 gene modules to about 80 gene modules, about 60 gene modules to about 70 gene modules, about 60 gene modules to about 80 gene modules, or about 70 gene modules to about 80 gene modules, that are most strongly correlated, among the plurality of gene modules, with the one or more sample traits. In some embodiments, the plurality of significant gene modules comprise about 10 gene modules to about 20 gene modules, about 10 gene modules to about 25 gene modules, about 10 gene modules to about 30 gene modules, about 10 gene modules to about 35 gene modules, about 10 gene modules to about 40 gene modules, about 10 gene modules to about 45 gene modules, about 10 gene modules to about 50 gene modules, about 10 gene modules to about 55 gene modules, about 10 gene modules to about 60 gene modules, about 10 gene modules to about 70 gene modules, about 10 gene modules to about 80 gene modules, about 20 gene modules to about 25 gene modules, about 20 gene modules to about 30 gene modules, about 20 gene modules to about 35 gene modules, about 20 gene modules to about 40 gene modules, about 20 gene modules to about 45 gene modules, about 20 gene modules to about 50 gene modules, about 20 gene modules to about 55 gene modules, about 20 gene modules to about 60 gene modules, about 20 gene modules to about 70 gene modules, about 20 gene modules to about 80 gene modules, about 25 gene modules to about 30 gene modules, about 25 gene modules to about 35 gene modules, about 25 gene modules to about 40 gene modules, about 25 gene modules to about 45 gene modules, about 25 gene modules to about 50 gene modules, about 25 gene modules to about 55 gene modules, about 25 gene modules to about 60 gene modules, about 25 gene modules to about 70 gene modules, about 25 gene modules to about 80 gene modules, about 30 gene modules to about 35 gene modules, about 30 gene modules to about 40 gene modules, about 30 gene modules to about 45 gene modules, about 30 gene modules to about 50 gene modules, about 30 gene modules to about 55 gene modules, about 30 gene modules to about 60 gene modules, about 30 gene modules to about 70 gene modules, about 30 gene modules to about 80 gene modules, about 35 gene modules to about 40 gene modules, about 35 gene modules to about 45 gene modules, about 35 gene modules to about 50 gene modules, about 35 gene modules to about 55 gene modules, about 35 gene modules to about 60 gene modules, about 35 gene modules to about 70 gene modules, about 35 gene modules to about 80 gene modules, about 40 gene modules to about 45 gene modules, about 40 gene modules to about 50 gene modules, about 40 gene modules to about 55 gene modules, about 40 gene modules to about 60 gene modules, about 40 gene modules to about 70 gene modules, about 40 gene modules to about 80 gene modules, about 45 gene modules to about 50 gene modules, about 45 gene modules to about 55 gene modules, about 45 gene modules to about 60 gene modules, about 45 gene modules to about 70 gene modules, about 45 gene modules to about 80 gene modules, about 50 gene modules to about 55 gene modules, about 50 gene modules to about 60 gene modules, about 50 gene modules to about 70 gene modules, about 50 gene modules to about 80 gene modules, about 55 gene modules to about 60 gene modules, about 55 gene modules to about 70 gene modules, about 55 gene modules to about 80 gene modules, about 60 gene modules to about 70 gene modules, about 60 gene modules to about 80 gene modules, or about 70 gene modules to about 80 gene modules, that are most strongly correlated, among the gene modules, with the one or more sample traits. In some embodiments, the plurality of significant gene modules comprise about 10 gene modules, about 20 gene modules, about 25 gene modules, about 30 gene modules, about 35 gene modules, about 40 gene modules, about 45 gene modules, about 50 gene modules, about 55 gene modules, about 60 gene modules, about 70 gene modules, or about 80 gene modules. In some embodiments, the plurality of significant gene modules comprise about 10 gene modules, about 20 gene modules, about 25 gene modules, about 30 gene modules, about 35 gene modules, about 40 gene modules, about 45 gene modules, about 50 gene modules, about 55 gene modules, about 60 gene modules, about 70 gene modules, or about 80 gene modules, that are most strongly correlated with the one or more sample traits. In some embodiments, the plurality of significant gene modules comprise about 10 gene modules, about 20 gene modules, about 25 gene modules, about 30 gene modules, about 35 gene modules, about 40 gene modules, about 45 gene modules, about 50 gene modules, about 55 gene modules, about 60 gene modules, about 70 gene modules, or about 80 gene modules, that are most strongly correlated, among the gene modules, with the one or more sample. In some embodiments, the plurality of significant gene modules comprise at least about 10 gene modules, about 20 gene modules, about 25 gene modules, about 30 gene modules, about 35 gene modules, about 40 gene modules, about 45 gene modules, about 50 gene modules, about 55 gene modules, about 60 gene modules, or about 70 gene modules. In some embodiments, the plurality of significant gene modules comprise at most about 20 gene modules, about 25 gene modules, about 30 gene modules, about 35 gene modules, about 40 gene modules, about 45 gene modules, about 50 gene modules, about 55 gene modules, about 60 gene modules, about 70 gene modules, or about 80 gene modules. In some embodiments, the plurality of significant gene modules comprise at least about 10 gene modules, about 20 gene modules, about 25 gene modules, about 30 gene modules, about 35 gene modules, about 40 gene modules, about 45 gene modules, about 50 gene modules, about 55 gene modules, about 60 gene modules, or about 70 gene modules, that are most strongly correlated with the one or more sample traits. In some embodiments, the plurality of significant gene modules comprise at most about 20 gene modules, about 25 gene modules, about 30 gene modules, about 35 gene modules, about 40 gene modules, about 45 gene modules, about 50 gene modules, about 55 gene modules, about 60 gene modules, about 70 gene modules, or about 80 gene modules, that are most strongly correlated with the one or more sample traits. In some embodiments, the plurality of significant gene modules comprise at least about 10 gene modules, about 20 gene modules, about 25 gene modules, about 30 gene modules, about 35 gene modules, about 40 gene modules, about 45 gene modules, about 50 gene modules, about 55 gene modules, about 60 gene modules, or about 70 gene modules, that are most strongly correlated, among the gene modules, with the one or more sample. In some embodiments, the plurality of significant gene modules comprise at most about 20 gene modules, about 25 gene modules, about 30 gene modules, about 35 gene modules, about 40 gene modules, about 45 gene modules, about 50 gene modules, about 55 gene modules, about 60 gene modules, about 70 gene modules, or about 80 gene modules, that are most strongly correlated, among the gene modules, with the one or more sample. In some embodiments, third generation gene modules are selected. In some embodiments, second, third, and/or fourth generation gene modules are selected. In some embodiments, third generation gene modules are selected, wherein the plurality of significant gene modules comprise 10 to 80 most strongly correlated third generation gene modules. In some embodiments, second, third and/or fourth generation gene modules are selected, wherein the plurality of significant gene modules comprise 10 to 80 most strongly correlated second, third, and/or fourth generation gene modules. In some embodiments, the second, third and/or fourth generation gene modules of the plurality of gene modules are correlated with the one or more sample traits, and the plurality of significant gene modules comprises second, third and/or fourth gene modules. In some embodiments, the second, third and/or fourth gene modules comprise the 20 to 50 gene modules that are most strongly correlated with the one or more sample traits among the second, third and/or fourth generation gene modules of the plurality of gene modules. In some embodiments, the third generation gene modules of the plurality of gene modules are correlated with the one or more sample traits, and the plurality of significant gene modules comprises third generation gene modules. In some embodiments, the third generation gene modules comprise the 20 to 50 gene modules that are most strongly correlated with the one or more sample traits among the third generation gene modules of the plurality of gene modules.

In some embodiments, one or more genes are determined to be redundant in consideration of gene expression data from a biological sample. In some embodiments, one or more genes are determined to be redundant in consideration of gene expression data from a plurality of biological samples. In some embodiments, one or more genes are determined to be redundant in consideration of gene expression data from a plurality of biological samples from reference patients or reference biological samples. In some embodiments, one or more genes are determined to be redundant, and are referred to as redundant genes. In some embodiments, one or more redundant genes are excluded from the methods described herein. In some embodiments, one or more redundant genes are excluded before defining gene sets. In some embodiments, one or more redundant genes are excluded before defining gene modules. In some embodiments, one or more redundant genes are excluded after defining gene modules. In some embodiments, one or more redundant genes are excluded before defining a plurality of gene modules. In some embodiments, one or more redundant genes are excluded after defining a plurality of gene modules. In some embodiments, one or more redundant genes are associated with a correlation coefficient greater than a threshold value as described herein. In some embodiments, the threshold value is 0.6, 0.65, 0.7, 0.75, 0.8, 0.85, 0.9 or 0.95.

Provided herein are methods for determining a gene set or gene module capable of classifying a disease or disorder of a patient. In some embodiments, analysis of gene expression data results in the categorization of genes as gene sets or gene modules. In some embodiments, analysis of gene expression data results in the identification of gene modules as described herein. In some embodiments, the gene modules as described herein are associated with a molecular endotype of a disease or disorder. In some embodiments, the plurality of gene modules as described herein are associated with a molecular endotype of a disease or disorder. In some embodiments, the gene modules as described herein are associated with a phenotype of a disease or disorder. In some embodiments, the plurality of gene modules as described herein are associated with a phenotype of a disease or disorder. In some embodiments, the disease or disorder described herein comprises a chronic condition, an inflammatory condition, an autoimmune condition, an arthritis, a rheumatoid arthritis (RA), an early inflammatory arthritis (EIA), an inflammatory arthritis, a psoriatic arthritis (PSA), a lupus arthritis, a rhupus, an osteoarthritis, a non-inflammatory arthritis, a pauci-inflammatory arthritis, or combinations thereof. In some embodiments, the disease or disorder described herein comprises a chronic condition, an inflammatory condition, an autoimmune condition, an arthritis, a lupus, a fibromyalgia, or combinations thereof. In some embodiments, the disease or disorder is a chronic condition. In some embodiments, the disease or disorder is an inflammatory condition. In some embodiments, the disease or disorder is an arthritis. In some embodiments, the disease or disorder is a rheumatoid arthritis. In some embodiments, the disease or disorder is a lupus.

In some embodiments, the disease or disorder is type of arthritis. In some embodiments, a type of arthritis is any disease or disorder that a person of skill in the art would consider is a representation of arthritis. In some embodiments, the disease or disorder is a type of the disease or disorder. In some embodiments, the gene set as described herein is capable of classifying a type of the disease or disorder of a patient.

In some embodiments, the methods described herein comprise a gene set as described herein. In some embodiments, the gene set is capable of classifying a disease or disorder of a patient. In some embodiments, the gene set is capable of classifying a first disease or disorder of the patient. In some embodiments, the gene set is capable of classifying a second disease or disorder of the patient. In some embodiments, the gene set is capable of classifying a third disease or disorder of the patient. In some embodiments, the gene set is capable of classifying a first disease or disorder, a second disease or disorder, or a third disease or disorder of the patient. In some embodiments, the gene set is capable of classifying whether the patient has a first disease or disorder, a second disease or disorder, or a third disease or disorder. In some embodiments, the gene set is capable of classifying a first disease or disorder, a second disease or disorder, or a third disease or disorder of a plurality of patients. In some embodiments, the gene set is capable of classifying a first disease or disorder, a second disease or disorder, or a third disease or disorder of a plurality of reference patients. In some embodiments, the gene set is capable of classifying the first plurality of patients as having the first disease or disorder. In some embodiments, the gene set is capable of classifying the second plurality of patients as having the second disease or disorder. In some embodiments, the gene set is capable of classifying the third plurality of patients as having the third disease or disorder. In some embodiments, the plurality of patients comprises the first plurality of patients having the first disease or disorder. In some embodiments, the plurality of patients comprises the second plurality of patients having the second disease or disorder. In some embodiments, the plurality of patients comprises the third plurality of patients having the third disease or disorder. In some embodiments, the plurality of patients comprises the first plurality of patients having the first disease or disorder, the second plurality of patients having the second disease or disorder, and the third plurality of patients having the third disease or disorder. In some embodiments, the plurality of patients comprises a first plurality of patients having the first disease or disorder and a second plurality of patients having the second disease or disorder, and the gene set is capable of classifying whether a patient has the first disease or disorder, or the second disease or disorder. In some embodiments, the plurality of patients comprises a plurality of reference patients. In some embodiments, the plurality of patients are classified as having a molecular endotype. In some embodiments, the first disease or disorder is a first molecular endotype. In some embodiments, the second disease or disorder is a second molecular endotype. In some embodiments, the third disease or disorder is a third molecular endotype. In some embodiments, a different disease or disorder corresponds to a different molecular endotype. In some embodiments, the gene set comprising gene expression data is capable of classifying a different molecular endotype. In some embodiments, the gene set comprising gene expression data is capable of classifying a different disease or disorder. In some embodiments, a different disease or disorder corresponds to a different phenotype.

In some embodiments, the method classifies the disease or disorder of the patient with an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method classifies the disease or disorder of the patient with a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method classifies the disease or disorder of the patient with a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method classifies the disease or disorder of the patient with a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method classifies the disease or disorder of the patient with a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method classifies the disease or disorder of the patient with an accuracy of about 85% to about 100%. In some embodiments, the method classifies the disease or disorder of the patient with an accuracy of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classifies the disease or disorder of the patient with an accuracy of about 85%, about 90%, about 92%, about 94%, about 95%, about 96%, about 98%, about 99%, about 99.3%, about 99.5%, about 99.8%, or about 100%. In some embodiments, the method classifies the disease or disorder of the patient with an accuracy of at least about 85%, about 90%, about 92%, about 94%, about 95%, about 96%, about 98%, about 99%, about 99.3%, about 99.5%, or about 99.8%.

In some embodiments, the method classifies the disease or disorder of the patient with a sensitivity of about 85% to about 100%. In some embodiments, the method classifies the disease or disorder of the patient with a sensitivity of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classifies the disease or disorder of the patient with a sensitivity of about 85%, about 90%, about 92%, about 94%, about 95%, about 96%, about 98%, about 99%, about 99.3%, about 99.5%, about 99.8%, or about 100%. In some embodiments, the method classifies the disease or disorder of the patient with a sensitivity of at least about 85%, about 90%, about 92%, about 94%, about 95%, about 96%, about 98%, about 99%, about 99.3%, about 99.5%, or about 99.8%.

In some embodiments, the method classifies the disease or disorder of the patient with a specificity of about 85% to about 100%. In some embodiments, the method classifies the disease or disorder of the patient with a specificity of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classifies the disease or disorder of the patient with a specificity of about 85%, about 90%, about 92%, about 94%, about 95%, about 96%, about 98%, about 99%, about 99.3%, about 99.5%, about 99.8%, or about 100%. In some embodiments, the method classifies the disease or disorder of the patient with a specificity of at least about 85%, about 90%, about 92%, about 94%, about 95%, about 96%, about 98%, about 99%, about 99.3%, about 99.5%, or about 99.8%.

In some embodiments, the method classifies the disease or disorder of the patient with a positive predictive value of about 85% to about 100%. In some embodiments, the method classifies the disease or disorder of the patient with a positive predictive value of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classifies the disease or disorder of the patient with a positive predictive value of about 85%, about 90%, about 92%, about 94%, about 95%, about 96%, about 98%, about 99%, about 99.3%, about 99.5%, about 99.8%, or about 100%. In some embodiments, the method classifies the disease or disorder of the patient with a positive predictive value of at least about 85%, about 90%, about 92%, about 94%, about 95%, about 96%, about 98%, about 99%, about 99.3%, about 99.5%, or about 99.8%.

In some embodiments, the method classifies the disease or disorder of the patient with a negative predictive value of about 85% to about 100%. In some embodiments, the method classifies the disease or disorder of the patient with a negative predictive value of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the method classifies the disease or disorder of the patient with a negative predictive value of about 85%, about 90%, about 92%, about 94%, about 95%, about 96%, about 98%, about 99%, about 99.3%, about 99.5%, about 99.8%, or about 100%. In some embodiments, the method classifies the disease or disorder of the patient with a negative predictive value of at least about 85%, about 90%, about 92%, about 94%, about 95%, about 96%, about 98%, about 99%, about 99.3%, about 99.5%, or about 99.8%.

In some embodiments, the gene modules comprise at least a minimum number of genes to obtain the desired accuracy, sensitivity, specificity, positive predictive value and/or negative predictive value in disease or disorder classification, such disease or disorder classification.

In some embodiments, the machine-learning model comprises the accuracy, sensitivity, specificity, positive predictive value, and/or negative predictive value, described above, and the accuracy, sensitivity, specificity, positive predictive value, and/or negative predictive value of the method is based on the classification parameters of the machine-learning model, as described herein and/or as understood by one of skill in the art.

### III. Methods Directed to a Disease or Disorder of a Patient

In some embodiments, the method described herein comprises identifying a disease or disorder as described herein, classifying a disease or disorder as described herein, identifying a patient's susceptibility to a disease or disorder as described herein, identifying the progression of the disease or disorder of the patient as described herein, identifying if the patient is likely to respond to a treatment as described herein, identifying if the patient is likely to respond to a treatment comprising administration of a drug as described herein, identifying an effectiveness of the treatment as compared to the progression of the disease or disorder of the patient as described herein, or combinations thereof.

In some embodiments, the method described herein comprises identifying a disease or disorder as described herein. In some embodiments, the method described herein comprises classifying a disease or disorder as described herein. In some embodiments, the method described herein comprises identifying a patient's susceptibility to a disease or disorder as described herein. In some embodiments, the method described herein comprises identifying the progression of the disease or disorder of the patient as described herein. In some embodiments, the method described herein comprises identifying if the patient is likely to respond to a treatment as described herein. In some embodiments, the method described herein comprises identifying if the patient is likely to respond to a treatment comprising administration of a drug as described herein. In some embodiments, the method described herein comprises identifying an effectiveness of the treatment as compared to the progression of the disease or disorder of the patient as described herein.

In some embodiments, the methods described herein comprise identifying, classifying, and/or treating a first disease or disorder, a second disease or disorder, or a third disease or disorder of the patient. In some embodiments, the methods described herein comprise identifying, classifying, and/or treating a first disease or disorder of the patient. In some embodiments, the methods described herein comprise identifying, classifying, and/or treating a second disease or disorder of the patient. In some embodiments, the methods described herein comprise identifying, classifying, and/or treating a third disease or disorder of the patient.

In some embodiments, the method comprises classifying a first disease or disorder, a second disease or disorder, or a third disease or disorder of the patient. In some embodiments, the method comprises classifying a first disease or disorder of the patient. In some embodiments, the method comprises classifying a second disease or disorder of the patient. In some embodiments, the method comprises classifying a third disease or disorder of the patient.

In some embodiments, the methods described herein comprise identifying, classifying, and/or treating a disease or disorder. In some embodiments, the methods described herein also comprise identifying the progression of the disease or disorder.

In some embodiments, the methods described herein comprise identifying, classifying, and/or treating a chronic condition, an inflammatory condition, an autoimmune condition, an arthritis, a rheumatoid arthritis (RA), an early inflammatory arthritis (EIA), an inflammatory arthritis, a psoriatic arthritis (PSA), a lupus arthritis, a rhupus, an osteoarthritis, a non-inflammatory arthritis, a pauci-inflammatory arthritis, or combinations thereof. In some embodiments, the methods described herein also comprise identifying the progression of the disease or disorder, the chronic condition, the inflammatory condition, the autoimmune condition, the arthritis, the rheumatoid arthritis (RA), the early inflammatory arthritis (EIA), the inflammatory arthritis, the psoriatic arthritis (PSA), the lupus arthritis, the rhupus, the osteoarthritis, the non-inflammatory arthritis, the pauci-inflammatory arthritis, or combinations thereof.

In some embodiments, the methods described herein comprise identifying, classifying, and/or treating an arthritis. In some embodiments, the methods described herein also comprise identifying the progression of the arthritis. In some embodiments, the method comprises classifying an arthritis of the patient.

In some embodiments, the methods described herein comprise identifying, classifying, and/or treating a rheumatoid arthritis (RA). In some embodiments, the methods described herein also comprise identifying the progression of the rheumatoid arthritis (RA).

In some embodiments, the method comprises the administration of a treatment to the patient in consideration of the classification of the disease or disorder of the patient. In some embodiments, the method comprises the classification of the disease or disorder of the patient. In some embodiments, the method comprises the administration of the treatment. In some embodiments, the treatment comprises the administration of a drug. In some embodiments, the treatment comprises administration of a treatment to the patient in consideration of the classification of the disease or disorder of the patient. In some embodiments, the treatment comprises administration of a drug to the patient in consideration of the classification of the disease or disorder of the patient.

In some embodiments, the method comprises identifying an arthritis of the patient. In some embodiments, the method comprises the classification of the arthritis of the patient. In some embodiments, the method comprises the administration of a treatment to the patient in consideration of the classification of the disease or disorder of the patient as a first disease, a second disease, or a third disease. In some embodiments, the treatment of the patient is directed to the first disease. In some embodiments, the treatment of the patient is directed to the second disease. In some embodiments, the treatment of the patient is directed to the third disease. In some embodiments, the treatment of the patient is directed to an arthritis. In some embodiments, the treatment of the patient is directed to a RA. In some embodiments, the treatment of the patient comprises the administration of a drug as described herein.

In some embodiments, the methods described herein comprise a data set. In some embodiments, the data set comprises or is derived from gene expression data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, 2000 or all genes, selected from the genes in any one of TABLE 2, TABLE 3, TABLE 4, or

TABLE 5, from the biological sample from the patient. In some embodiments, the data set comprises or is derived from gene expression data of at least 2 genes, selected from the genes in any one of TABLE 2, TABLE 3, TABLE 4, or TABLE 5, from the biological sample from the patient.

In some embodiments, the data set comprises or is derived from gene expression data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, 2000 or all genes, selected from the genes modules in any one of TABLE 2, TABLE 3, TABLE 4, or TABLE 5, from the biological sample from the patient. In some embodiments, the data set comprises or is derived from gene expression data of at least 2 genes, selected from the genes modules in any one of TABLE 2, TABLE 3, TABLE 4, or TABLE 5, from the biological sample from the patient.

In some embodiments, the genes selected from different Tables as described herein are different. In some embodiments, the genes selected from different Tables as described herein are the same.

In some embodiments, the data set comprises or is derived from gene expression data of effective number of genes selected from the genes in any one of TABLE 2, TABLE 3, TABLE 4, or TABLE 5, from the biological sample from the patient, wherein number of genes selected from different Tables may be different or the same. In some embodiments, the data set comprises or is derived from gene expression data of all genes in any one of TABLE 2, TABLE 3, TABLE 4, or TABLE 5. In some embodiments, the data set comprises or is derived from gene expression data of all genes in one or more of TABLE 2, TABLE 3, TABLE 4, or TABLE 5. In some embodiments, the data set comprises or is derived from gene expression data of all genes in one or more of Tables described herein. In some embodiments, the one or more Tables comprise at least 2, 3, 4, 5, 6, 7, 8, 9, 10, 11, 12, 13, 14, 15, 17, 18, 19, 20, 21, 22, 23, 24, 25, 27, 28, 29, or 30 Tables. In some embodiments, the one or more Tables comprise at least 2, 3, 4, 5, 6, 7, 8, 9, 10, 11, 12, 13, 14, 15, 17, 18, 19, 20, 21, 22, 23, 24, 25, 27, 28, 29, or 30 Tables selected from a list comprising TABLE 2, TABLE 3, TABLE 4, and TABLE 5. In some embodiments, Tables described herein are selected from a list comprising TABLE 2, TABLE 3, TABLE 4, and TABLE 5.

In some embodiments, the method comprises the genes modules in any one of TABLE 2, TABLE 3, TABLE 4, or TABLE 5, from the biological sample from the patient.

In some embodiments, the method comprises module eigengenes (MEs). In some embodiments, the method comprises a data set comprises MEs. In some embodiments, the MEs comprise gene expression data. In some embodiments, the MEs comprise gene expression data of genes in any one of TABLE 2, TABLE 3, TABLE 4, or TABLE 5. In some embodiments, the MEs comprise gene expression data of genes in one or more of TABLE 2, TABLE 3, TABLE 4, or TABLE 5. In some embodiments, the MEs comprise gene expression profiles in a gene module. In some embodiments, the MEs comprise gene expression profiles in a gene module comprising gene coexpression data. In some embodiments, MEs identify the relationships between gene modules. In some embodiments, the method comprises the correlation of the one or more gene modules with one or more sample traits comprises correlating the MEs. In some embodiments, the genes modules in any one of TABLE 2, TABLE 3, TABLE 4, or TABLE 5. In some embodiments, the MEs comprise gene expression data of genes modules in any one of TABLE 2, TABLE 3, TABLE 4, or TABLE 5. In some embodiments, the MEs comprise gene expression data of gene modules in one or more of TABLE 2, TABLE 3, TABLE 4, or TABLE 5. In some embodiments, the data set comprises MEs comprising gene expression data of genes modules in any one of TABLE 2, TABLE 3, TABLE 4, or TABLE 5.

In some embodiments, the data set is derived from the gene expression data using GSVA, wherein the data set comprises one or more GSVA scores of the patient, wherein the one or more GSVA scores are generated based on the gene expression data of genes or gene modules in one or more of Tables described herein. In some embodiments, the data set is derived from the gene expression data using GSVA, wherein the data set comprises one or more GSVA scores of the patient, wherein the one or more GSVA scores are generated based on the gene expression data of genes or gene modules in any one of TABLE 2, TABLE 3, TABLE 4, or TABLE 5. In some embodiments, the data set is derived from the gene expression data using GSVA, wherein the data set comprises one or more GSVA scores of the patient, wherein the one or more GSVA scores are generated based on the gene expression data of genes or gene modules in one or more of TABLE 2, TABLE 3, TABLE 4, or TABLE 5. In some embodiments, at least one GSVA score of the patient is generated based on enrichment of gene expression data of the genes selected from a Table described herein. In some embodiments, the one or more GSVA scores comprise each generated GSVA score.

In some embodiments, for each selected Table, the at least one GSVA score of the patient is generated based on enrichment of expression of an effective number of genes selected from the genes listed in the selected Table, in the biological sample.

In some embodiments, analyzing the data set comprises providing the data set as an input to a machine learning model trained to generate an inference of whether the data set is indicative of the patient having a disease or disorder. In some embodiments, the inference is used in classifying that the patient has a disease or disorder.

In some embodiments, the methods described herein comprise classifying the disease or disorder of the patient based on the inference. In some embodiments, the data set comprises the one or more GSVA scores of the patient, and the machine learning model generate the inference based at least on the one or more GSVA scores. In some embodiments, the data set comprises the MEs, and the machine learning model generate the inference based at least on the MEs. In some embodiments, the method further comprises receiving, as an output of the trained machine learning model, the inference; and/or electronically outputting a report indicating the first disease or disorder disease or disorder of the patient based on the inference. In some embodiments, the report comprises nucleic acid sequencing data, transcriptome data, genome data, epigenetic data, proteome data, metabolome data, virome data, metabolome data, methylome data, lipidomic data, lineage-ome data, nucleosomal occupancy data, a genetic variant, a gene fusion, an indel, or combinations thereof.

In some embodiments, analyzing the data set comprises generating a disease risk score of the patient based on the data set, and classifying whether the data set is indicative of the patient having the disease or disorder based on the disease risk score. The disease risk score of the patient is generated based on the one or more GSVA scores of the patient. In some embodiments, the methods described herein comprise classifying the disease or disorder of the patient with an accuracy of at least 85%. In some embodiments, the methods described herein comprise classifying the disease or disorder of the patient with a sensitivity of at least 85%. In some embodiments, the methods described herein comprise classifying the disease or disorder of the patient with a specificity of at least 85%. In some embodiments, the methods described herein comprise classifying the disease or disorder of the patient with a positive predictive value of at least 85%. In some embodiments, the methods described herein comprise classifying the disease or disorder of the patient with a negative predictive value of at least 85%.

In some embodiments, the methods described herein comprise classifying a disease or disorder of a patient by analyzing a data set comprising or derived from gene expression data of at least 2 genes selected from the genes listed in as shown in TABLE 2, TABLE 3, TABLE 4, or TABLE 5, from a biological sample obtained or derived from the patient, to classify the disease or disorder of the patient. In some embodiments, classifying the disease or disorder of the patient can comprise classifying (e.g., determining) whether the patient has the disease or disorder. In some embodiments, the data set comprises or is derived from gene expression data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, 2000 or all genes, selected from the genes listed in as shown in TABLE 2, TABLE 3, TABLE 4, or TABLE 5, from the biological sample from the patient.

In some embodiments, the data set comprises or is derived from gene expression data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, 2000, or any number of the genes listed in TABLE 2, TABLE 3, TABLE 4, or TABLE 5, from the biological sample from the patient.

In some embodiments, the data set is derived from the gene expression data using gene set variation analysis (GSVA), gene set enrichment analysis (GSEA), enrichment algorithm, multiscale embedded gene coexpression network analysis (MEGENA), weighted gene coexpression network analysis (WGCNA), differential expression analysis, Z-score, log 2 expression analysis, or any combination thereof. In some embodiments, the data set is derived from the gene expression data using GSVA. In some embodiments, the data set is derived from the gene expression data using GSVA, wherein the data set comprises one or more GSVA scores of the patient, wherein the one or more GSVA scores are generated based on genes listed in TABLE 2, TABLE 3, TABLE 4, or TABLE 5, wherein for each selected Table, at least one GSVA score of the patient is generated based on enrichment of expression of the genes selected from the selected Table, in the biological sample, and wherein the one or more GSVA scores comprise each generated GSVA score.

In some embodiments, the methods described herein comprise determining different patient populations, different patient treatment groups, and/or different patient subsets. In some embodiments, different patient populations, different patient treatment groups, and/or different patient subsets are determined in consideration of at least one gene set variation analysis (GSVA). In some embodiments, different patient populations, different patient treatment groups, and/or different patient subsets are determined in consideration of GSVA scores and k-means clustering method. In some embodiments, different patient populations, different patient treatment groups, and/or different patient subsets are determined in consideration of module eigengenes (MEs) of the significant gene clusters as described herein. In some embodiments, different patient populations, different patient treatment groups, and/or different patient subsets are determined in consideration of MEs and k-means clustering method. In some embodiments, different patient populations, different patient treatment groups, and/or different patient subsets are determined in consideration of a machine learning model as described herein. In some embodiments, the machine learning model is trained as described herein, and/or as understood by one of ordinary skill in the art.

In some embodiments, the methods described herein comprise classifying a disease or disorder of a patient. In some embodiments, the methods comprise classifying a disease or disorder of a patient comprises: analyzing a data set comprising gene expression data. In some embodiments, the methods comprise gene expression data of at least 2 genes associated with a gene module or a plurality of significant gene modules. In some embodiments, the methods comprise gene expression data of at least 2 genes from a sample from the patient to classify the disease or disorder of the patient.

In some embodiments, the methods described herein comprise gene expression data of at least 2 genes selected from the genes listed within a gene set. In some embodiments, the methods described herein comprise gene expression data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, 2000 or all genes, selected from the genes within a gene set.

In some embodiments, the methods described herein comprise gene expression data of at least 2 genes selected from the genes listed within the gene module. In some embodiments, the methods described herein comprise gene expression data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 450, 500, 550, 600, 650, 700, 750, 850, 900, 950, 1000, 1050, 1100, 1150, 1200, 1250, 1300, 1350, 1400, 1450, 1500, 1550, 1600, 1700, 1800, 1900, 2000 or all genes, selected from the genes within a gene module.

In some embodiments, the methods described herein comprise gene expression data of at least 2 genes from the genes within a gene module as described herein. In some embodiments, the methods described herein comprise gene expression data of genes within one or more gene modules as described herein. In some embodiments, the one or more gene modules are selected from the significant gene modules. In some embodiments, the methods described herein comprise gene expression data of genes selected from TABLE 2, TABLE 3, TABLE 4, or TABLE 5. In some embodiments, the methods described herein comprise gene expression data of genes associated with a gene module selected from TABLE 2, TABLE 3, TABLE 4, or TABLE 5. In some embodiments, the methods described herein comprise gene expression data of genes associated with a plurality of gene modules selected from TABLE 2, TABLE 3, TABLE 4, or TABLE 5. In some embodiments, the methods described herein comprise gene expression data of an effective number of genes associated with a plurality of significant gene modules selected from TABLE 2, TABLE 3, TABLE 4, or TABLE 5.

In some embodiments, the methods described herein comprise classifying a disease or disorder of a patient by analyzing a data set comprising or derived from gene expression data of at least 2 genes selected from the genes modules in any one of TABLE 2, TABLE 3, TABLE 4, or TABLE 5. In some embodiments, the method comprises analyzing a data set comprising gene expression data from a biological sample isolated from the patient, to classify the disease or disorder of the patient. In some embodiments, classifying the disease or disorder of the patient comprises the classification of the disease or disorder of the patient as a first disease, a second disease, or a third disease. In some embodiments, classifying the disease or disorder of the patient comprises the classification of the disease or disorder of the patient as an arthritis, a rheumatoid arthritis (RA), an early inflammatory arthritis (EIA), an inflammatory arthritis, a psoriatic arthritis (PSA), a lupus arthritis, a rhupus, an osteoarthritis, a non-inflammatory arthritis, or a pauci-inflammatory arthritis. In some embodiments, classifying the disease or disorder of the patient comprises the classification of the disease or disorder of the patient as an arthritis. In some embodiments, classifying the disease or disorder of the patient comprises the classification of the disease or disorder of the patient as a rheumatoid arthritis (RA).

In some embodiments, the methods described herein comprise selecting an effective number of genes from a Table/module (e.g., a Table from TABLE 2, TABLE 3, TABLE 4, or TABLE 5) comprises selecting at least minimum number of genes from the Table/module to obtain desired accuracy, sensitivity, specificity, positive predictive value, and/or negative predictive value in classification of the disease or disorder of the patient. In some embodiments, desired accuracy, sensitivity, specificity, positive predictive value, and/or negative predictive value, is an accuracy, sensitivity, specificity, positive predictive value, and/or negative predictive value respectively described herein. In some embodiments, the desired accuracy, sensitivity, specificity, positive predictive value, and/or negative predictive value, is at least 85%. In some embodiments, the desired accuracy, sensitivity, specificity, positive predictive value, and/or negative predictive value, is at least 90%. In some embodiments, the desired accuracy, sensitivity, specificity, positive predictive value, and/or negative predictive value, is at least 95%. In some embodiments, effective number of genes for a module/Table are determined using adjusted rand index (ARI) method. In some embodiments, effective number of genes for a module/Table are determined by performing k-means clustering on randomly selected groups of genes by standard interval based on the total number of genes of the respective Table/module. In some embodiments, similarity is measured by adjusted rand index (ARI). In some embodiments, the adjusted rand index (ARI) is calculated between K-Means module memberships from each randomly selected group of genes to the module memberships obtained using total number of genes of the respective Table/module. In some embodiments, the higher the ARI, the stronger the module memberships suggesting sufficient genes are selected. In some embodiments, the lower the ARI the weaker the module memberships suggesting more genes are required. In some embodiments, the ARI is calculated to determine the effective number of genes for each Table/module selected. In some embodiments, selecting the effective number of genes from a Table (e.g., a Table from TABLE 2, TABLE 3, TABLE 4, or TABLE 5) comprises selecting at least 60%, 70%, 80%, 90%, or all genes from the Table. In some embodiments, selecting effective number of genes from a Table (e.g., a Table from TABLE 2, TABLE 3, TABLE 4, or TABLE 5) comprises selecting at least 60%, 70%, 80%, 90%, or all genes from the Table, where the Table comprises 100 or more genes. In some embodiments, selecting effective number of genes from a Table (e.g., a Table from TABLE 2, TABLE 3, TABLE 4, or TABLE 5) comprises selecting at least 70%, genes from the Table, where the Table comprises 100 or more genes. In some embodiments, selecting effective number of genes from a Table (e.g., a Table from TABLE 2, TABLE 3, TABLE 4, or TABLE 5) comprises selecting at least 80%, 90%, 95% or all genes from the Table, where the Table comprises less than 100 genes. In some embodiments, selecting effective number of genes from a Table (e.g., a Table from TABLE 2, TABLE 3, TABLE 4, or TABLE 5) comprises selecting all genes from the Table, where the Table comprises less than 100 genes. In some embodiments, at least a minimum number of genes from the Tables (e.g., from TABLE 2, TABLE 3, TABLE 4, or TABLE 5, such as based on the absolute coefficient value of the Tables) are selected, such that the method classifies the disease or disorder of the patient with desired accuracy, sensitivity, specificity, positive predictive value and/or negative predictive value, such as at least 85% accuracy, at least 85% sensitivity, at least 85% specificity, at least 85% positive predictive value and/or at least 85% negative predictive value. In some embodiments, an effective number of genes from each of the selected Tables (e.g., from TABLE 2, TABLE 3, TABLE 4, or TABLE 5) are selected, such that the method classifies the disease or disorder of the patient with desired accuracy, sensitivity, specificity, positive predictive value and/or negative predictive value, such as at least 85% accuracy, at least 85% sensitivity, at least 85% specificity, at least 85% positive predictive value and/or at least 85% negative predictive value. In some embodiments, at least a minimum number of genes from the Tables (e.g., from TABLE 2, TABLE 3, TABLE 4, or TABLE 5, such as based on the absolute coefficient value of the Tables) and an effective number of genes from each of the selected Tables are selected, such that the method classifies the disease or disorder of the patient with desired accuracy, sensitivity, specificity, positive predictive value and/or negative predictive value, such as at least 85% accuracy, at least 85% sensitivity, at least 85% specificity, at least 85% positive predictive value and/or at least 85% negative predictive value.

Data Analysis Methods and Machine learning Model

In some embodiments, the methods described herein comprise gene modules as described herein. In some embodiments, the gene modules comprise the significant gene modules of the gene set. In some embodiments, the gene modules comprise the significant gene modules of the gene set. In some embodiments, the methods described herein comprise data analysis and a machine learning model. In some embodiments, the methods described herein comprise more than one data analysis method described herein and the machine learning model described herein. In some embodiments, the methods described herein comprise a GVSA score. In some embodiments, the methods described herein comprise the GVSA score and the machine learning model described herein.

In some embodiments, the methods described herein comprise a data set as described herein. In some embodiments, the data set is derived from the gene expression data using gene set variation analysis (GSVA), gene set enrichment analysis (GSEA), enrichment algorithm, multiscale embedded gene coexpression network analysis (MEGENA), weighted gene coexpression network analysis (WGCNA), differential expression analysis, Z-score, log 2 expression analysis, or any combination thereof. In some embodiments, the data set is derived from the gene expression data using GSVA. In some embodiments, the data set comprises one or more GSVA scores.

In some embodiments, the data set as described herein is a patient data set. In some embodiments, the patient data set is derived from the gene expression data using gene set variation analysis (GSVA), gene set enrichment analysis (GSEA), enrichment algorithm, multiscale embedded gene coexpression network analysis (MEGENA), weighted gene coexpression network analysis (WGCNA), differential expression analysis, Z-score, log 2 expression analysis, or any combination thereof. In some embodiments, the patient data set is derived from the gene expression data using GSVA. In some embodiments, the patient data set comprises one or more GSVA scores of the patient.

In some embodiments, the one or more GSVA scores as described herein are generated based on one or more gene modules selected from the significant gene modules of the gene set. In some embodiments, a selected module is one of the one or more gene modules selected from the significant gene modules. In some embodiments, a GVSA score is generated based on enrichment of gene expression data as described herein. In some embodiments, a GVSA score is generated based on enrichment of gene expression data from genes within a gene module. In some embodiments, a GVSA score is generated based on enrichment of gene expression data from one or more gene modules. In some embodiments, a GVSA score is generated based on enrichment of gene expression data from one or more significant gene modules. In some embodiments, a GVSA score is generated based on enrichment of gene expression data from a plurality of gene modules.

In some embodiments, a GVSA score is generated based on enrichment of gene expression data of at least 2 genes. In some embodiments, a GVSA score is generated based on enrichment of gene expression data of at least 2 genes associated with the gene module. In some embodiments, a GVSA score is generated based on enrichment of gene expression data of at least 2 genes associated with one or more gene module. In some embodiments, a GVSA score is generated based on enrichment of gene expression data of at least 2 genes associated with the plurality of gene modules.

In some embodiments, a GVSA score is generated based on enrichment of gene expression data from a sample. In some embodiments, a GVSA score is generated based on enrichment of gene expression data from a biological sample. In some embodiments, a GVSA score is generated based on enrichment of gene expression data from an isolated biological sample from the patient.

In some embodiments, at least one GSVA score of the patient is generated based on enrichment of gene expression data of at least 2 genes associated with the selected gene module. In some embodiments, the one or more GVSA scores as described herein also referred to as a generated GSVA score. In some embodiments, the one or more GVSA scores comprise a generated GVSA score.

In some embodiments, at least one GSVA score of the patient is generated based on enrichment of gene expression data of an effective number of genes selected from the genes listed in the selected gene module. In some embodiments, the selected gene module comprises genes present in the biological sample. In some embodiments, the selected gene module comprises genes that are equal from the genes present in the biological sample. In some embodiments, the selected gene module comprises genes that are different from the genes present in the biological sample.

In some embodiments, the methods described herein comprise analyzing the data set as described herein. In some embodiments, analyzing the data set comprises inputting the data set into a machine learning model. In some embodiments, analyzing the data set comprises providing the data set as an input to a machine learning model. In some embodiments, the machine learning model is trained to generate an inference indicative of a disease of disorder.

In some embodiments, analyzing the data set comprises providing the data set as an input to a machine learning model trained to generate an inference of whether the data set is indicative of the patient having a disease or disorder. In some embodiments, analyzing the data set comprises providing the data set as an input to a machine learning model trained to generate an inference of whether the data set is indicative of a patient having a disease or disorder. In some embodiments, the data set comprises the one or more GSVA scores of the patient, and the machine learning model generate the inference based at least on the one or more GSVA scores.

In some embodiments, analyzing the data set comprises providing the data set as an input to a machine learning model trained to generate an inference of whether the data set is indicative of the patient having a disease or disorder. In some embodiments, analyzing the data set comprises providing the data set as an input to a machine learning model trained to generate an inference of whether the data set is indicative of the patient having a disease or disorder. In some embodiments, the inference is used in classifying that the patient has a disease or disorder.

In some embodiments, analyzing the patient data set comprises providing the patient data set as an input to a machine learning model trained to generate an inference of whether the patient data set is indicative of the patient having a disease or disorder. In some embodiments, the analyzing the patient data set comprises providing the patient data set as an input to a machine learning model trained to generate an inference of whether the patient data set is indicative of the patient having the disease or disorder. In some embodiments, the patient data set comprises the one or more GSVA scores of the patient, and the machine learning model generate the inference based at least on the one or more GSVA scores.

In some embodiments, the methods described herein comprise receiving, as an output of the machine learning model, the inference; and/or electronically outputting a report indicating the disease or disorder of the patient based on the inference.

In some embodiments, the inference from the machine learning model comprises a confidence value between 0 and 1, such as, 0, 0.1, 0.2, 0.3, 0.4, 0.5, 0.6, 0.7, 0.8, 0.9 or 1, or any value or ranges there between. In some embodiments, the higher confidence values are correlated with a higher likelihood. In some embodiments, the inference from the machine learning model comprises a confidence value between 0 and 1, such as, 0, 0.1, 0.2, 0.3, 0.4, 0.5, 0.6, 0.7, 0.8, 0.9 or 1, or any value or ranges there between, such that the inference is indicative of a disease or disorder. In some embodiments, the inference from the machine learning model can comprise a confidence value between 0 and 1, such as, 0, 0.1, 0.2, 0.3, 0.4, 0.5, 0.6, 0.7, 0.8, 0.9 or 1, or any value or ranges there between, indicating the disease or disorder of a patient. In some embodiments, the inference from the machine learning model can comprise a confidence value between 0 and 1, such as, 0, 0.1, 0.2, 0.3, 0.4, 0.5, 0.6, 0.7, 0.8, 0.9 or 1, or any value or ranges there between, indicating the patient having a disease or disorder.

In some embodiments, the machine learning model has a receiver operating characteristic (ROC) curve with an Area-Under-Curve (AUC) at least 0.85. In some embodiments, the methods described herein are directed to a method of training a machine learning model. In some embodiments, the machine learning model is trained as described herein, resulting in a trained machine learning model.

In some embodiments, the machine learning model is trained using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.

In some embodiments, the machine learning model is trained using linear regression. In some embodiments, the machine learning model is trained using logistic regression (LOG). In some embodiments, the machine learning model is trained using Ridge regression. In some embodiments, the machine learning model is trained using Lasso regression. In some embodiments, the machine learning model is trained using elastic net (EN) regression. In some embodiments, the machine learning model is trained using support vector machine (SVM). In some embodiments, the machine learning model is trained using gradient boosted machine (GBM). In some embodiments, the machine learning model is trained using k nearest neighbors (kNN). In some embodiments, the machine learning model is trained using generalized linear model (GLM). In some embodiments, the machine learning model is trained using naïve Bayes (NB) classifier. In some embodiments, the machine learning model is trained using neural network. In some embodiments, the machine learning model is trained using Random Forest (RF). In some embodiments, the machine learning model is trained using deep learning algorithm, linear discriminant analysis (LDA). In some embodiments, the machine learning model is trained using decision tree learning (DTREE). In some embodiments, the machine learning model is trained using adaptive boosting (ADB). In some embodiments, the machine learning model is trained using Classification and Regression Tree (CART). In some embodiments, the machine learning model is trained using hierarchical clustering.

In some embodiments, the trained machine learning model has an accuracy of at least about 90%, at least about 91%, at least about 92%, at least about 93%, at least about 94%, at least about 95%, at least about 96%, at least about 97%, at least about 98%, at least about 99%, at least about 99%, or more than about 99.5%. In some embodiments, the trained machine learning model has a sensitivity of at least about 90%, at least about 91%, at least about 92%, at least about 93%, at least about 94%, at least about 95%, at least about 96%, at least about 97%, at least about 98%, at least about 99%, or more than about 99%. In some embodiments, the trained machine learning model has a specificity of at least about 90%, at least about 91%, at least about 92%, at least about 93%, at least about 94%, at least about 95%, at least about 96%, at least about 97%, at least about 98%, at least about 99%, or more than about 99%. In some embodiments, the trained machine learning model has a positive predictive value of at least about 90%, at least about 91%, at least about 92%, at least about 93%, at least about 94%, at least about 95%, at least about 96%, at least about 97%, at least about 98%, at least about 99%, or more than about 99%. In some embodiments, the trained machine learning model has a negative predictive value of at least about 90%, at least about 91%, at least about 92%, at least about 93%, at least about 94%, at least about 95%, at least about 96%, at least about 97%, at least about 98%, at least about 99%, or more than about 99%.

In some embodiments, the trained machine learning model has an accuracy of about 85% to about 100%. In some embodiments, the trained machine learning model has an accuracy of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the trained machine learning model has an accuracy of about 85%, about 90%, about 92%, about 94%, about 95%, about 96%, about 98%, about 99%, about 99.3%, about 99.5%, about 99.8%, or about 100%. In some embodiments, the trained machine learning model has an accuracy of at least about 85%, about 90%, about 92%, about 94%, about 95%, about 96%, about 98%, about 99%, about 99.3%, about 99.5%, or about 99.8%. In some embodiments, the trained machine learning model has an accuracy of at most about 90%, about 92%, about 94%, about 95%, about 96%, about 98%, about 99%, about 99.3%, about 99.5%, about 99.8%, or about 100%.

In some embodiments, the trained machine learning model has a sensitivity of about 85% to about 100%. In some embodiments, the trained machine learning model has a sensitivity of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the trained machine learning model has a sensitivity of about 85%, about 90%, about 92%, about 94%, about 95%, about 96%, about 98%, about 99%, about 99.3%, about 99.5%, about 99.8%, or about 100%. In some embodiments, the trained machine learning model has a sensitivity of at least about 85%, about 90%, about 92%, about 94%, about 95%, about 96%, about 98%, about 99%, about 99.3%, about 99.5%, or about 99.8%. In some embodiments, the trained machine learning model has a sensitivity of at most about 90%, about 92%, about 94%, about 95%, about 96%, about 98%, about 99%, about 99.3%, about 99.5%, about 99.8%, or about 100%.

In some embodiments, the trained machine learning model has a specificity of about 85% to about 100%. In some embodiments, the trained machine learning model has a specificity of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the trained machine learning model has a specificity of about 85%, about 90%, about 92%, about 94%, about 95%, about 96%, about 98%, about 99%, about 99.3%, about 99.5%, about 99.8%, or about 100%. In some embodiments, the trained machine learning model has a specificity of at least about 85%, about 90%, about 92%, about 94%, about 95%, about 96%, about 98%, about 99%, about 99.3%, about 99.5%, or about 99.8%. In some embodiments, the trained machine learning model has a specificity of at most about 90%, about 92%, about 94%, about 95%, about 96%, about 98%, about 99%, about 99.3%, about 99.5%, about 99.8%, or about 100%.

In some embodiments, the trained machine learning model has a positive predictive value of about 85% to about 100%. In some embodiments, the trained machine learning model has a positive predictive value of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the trained machine learning model has a positive predictive value of about 85%, about 90%, about 92%, about 94%, about 95%, about 96%, about 98%, about 99%, about 99.3%, about 99.5%, about 99.8%, or about 100%. In some embodiments, the trained machine learning model has a positive predictive value of at least about 85%, about 90%, about 92%, about 94%, about 95%, about 96%, about 98%, about 99%, about 99.3%, about 99.5%, or about 99.8%. In some embodiments, the trained machine learning model has a positive predictive value of at most about 90%, about 92%, about 94%, about 95%, about 96%, about 98%, about 99%, about 99.3%, about 99.5%, about 99.8%, or about 100%.

In some embodiments, the trained machine learning model has a negative predictive value of about 85% to about 100%. In some embodiments, the trained machine learning model has a negative predictive value of about 85% to about 90%, about 85% to about 92%, about 85% to about 94%, about 85% to about 95%, about 85% to about 96%, about 85% to about 98%, about 85% to about 99%, about 85% to about 99.3%, about 85% to about 99.5%, about 85% to about 99.8%, about 85% to about 100%, about 90% to about 92%, about 90% to about 94%, about 90% to about 95%, about 90% to about 96%, about 90% to about 98%, about 90% to about 99%, about 90% to about 99.3%, about 90% to about 99.5%, about 90% to about 99.8%, about 90% to about 100%, about 92% to about 94%, about 92% to about 95%, about 92% to about 96%, about 92% to about 98%, about 92% to about 99%, about 92% to about 99.3%, about 92% to about 99.5%, about 92% to about 99.8%, about 92% to about 100%, about 94% to about 95%, about 94% to about 96%, about 94% to about 98%, about 94% to about 99%, about 94% to about 99.3%, about 94% to about 99.5%, about 94% to about 99.8%, about 94% to about 100%, about 95% to about 96%, about 95% to about 98%, about 95% to about 99%, about 95% to about 99.3%, about 95% to about 99.5%, about 95% to about 99.8%, about 95% to about 100%, about 96% to about 98%, about 96% to about 99%, about 96% to about 99.3%, about 96% to about 99.5%, about 96% to about 99.8%, about 96% to about 100%, about 98% to about 99%, about 98% to about 99.3%, about 98% to about 99.5%, about 98% to about 99.8%, about 98% to about 100%, about 99% to about 99.3%, about 99% to about 99.5%, about 99% to about 99.8%, about 99% to about 100%, about 99.3% to about 99.5%, about 99.3% to about 99.8%, about 99.3% to about 100%, about 99.5% to about 99.8%, about 99.5% to about 100%, or about 99.8% to about 100%. In some embodiments, the trained machine learning model has a negative predictive value of about 85%, about 90%, about 92%, about 94%, about 95%, about 96%, about 98%, about 99%, about 99.3%, about 99.5%, about 99.8%, or about 100%. In some embodiments, the trained machine learning model has a negative predictive value of at least about 85%, about 90%, about 92%, about 94%, about 95%, about 96%, about 98%, about 99%, about 99.3%, about 99.5%, or about 99.8%. In some embodiments, the trained machine learning model has a negative predictive value of at most about 90%, about 92%, about 94%, about 95%, about 96%, about 98%, about 99%, about 99.3%, about 99.5%, about 99.8%, or about 100%.

In some embodiments, the trained machine learning model has a receiver operating characteristic (ROC) curve with an Area-Under-Curve (AUC) at least about 0.90, at least about 0.91, at least about 0.92, at least about 0.93, at least about 0.94, at least about 0.95, at least about 0.96, at least about 0.97, at least about 0.98, at least about 0.99, or more than about 0.99. In some embodiments, the trained machine learning model has a ROC curve with an AUC of about 0.85 to about 1. In some embodiments, the trained machine learning model has a ROC curve with an AUC of about 0.85 to about 0.9, about 0.85 to about 0.92, about 0.85 to about 0.94, about 0.85 to about 0.95, about 0.85 to about 0.96, about 0.85 to about 0.98, about 0.85 to about 0.99, about 0.85 to about 0.993, about 0.85 to about 0.995, about 0.85 to about 0.998, about 0.85 to about 1, about 0.9 to about 0.92, about 0.9 to about 0.94, about 0.9 to about 0.95, about 0.9 to about 0.96, about 0.9 to about 0.98, about 0.9 to about 0.99, about 0.9 to about 0.993, about 0.9 to about 0.995, about 0.9 to about 0.998, about 0.9 to about 1, about 0.92 to about 0.94, about 0.92 to about 0.95, about 0.92 to about 0.96, about 0.92 to about 0.98, about 0.92 to about 0.99, about 0.92 to about 0.993, about 0.92 to about 0.995, about 0.92 to about 0.998, about 0.92 to about 1, about 0.94 to about 0.95, about 0.94 to about 0.96, about 0.94 to about 0.98, about 0.94 to about 0.99, about 0.94 to about 0.993, about 0.94 to about 0.995, about 0.94 to about 0.998, about 0.94 to about 1, about 0.95 to about 0.96, about 0.95 to about 0.98, about 0.95 to about 0.99, about 0.95 to about 0.993, about 0.95 to about 0.995, about 0.95 to about 0.998, about 0.95 to about 1, about 0.96 to about 0.98, about 0.96 to about 0.99, about 0.96 to about 0.993, about 0.96 to about 0.995, about 0.96 to about 0.998, about 0.96 to about 1, about 0.98 to about 0.99, about 0.98 to about 0.993, about 0.98 to about 0.995, about 0.98 to about 0.998, about 0.98 to about 1, about 0.99 to about 0.993, about 0.99 to about 0.995, about 0.99 to about 0.998, about 0.99 to about 1, about 0.993 to about 0.995, about 0.993 to about 0.998, about 0.993 to about 1, about 0.995 to about 0.998, about 0.995 to about 1, or about 0.998 to about 1. In some embodiments, the trained machine learning model has a ROC curve with an AUC of about 0.85, about 0.9, about 0.92, about 0.94, about 0.95, about 0.96, about 0.98, about 0.99, about 0.993, about 0.995, about 0.998, or about 1. In some embodiments, the trained machine learning model has a ROC curve with an AUC of at least about 0.85, about 0.9, about 0.92, about 0.94, about 0.95, about 0.96, about 0.98, about 0.99, about 0.993, about 0.995, or about 0.998. In some embodiments, the trained machine learning model has a ROC curve with an AUC of at most about 0.9, about 0.92, about 0.94, about 0.95, about 0.96, about 0.98, about 0.99, about 0.993, about 0.995, about 0.998, or about 1.

For example, in some embodiments, patient data comprises ranges of patient data (e.g., gene expression data and/or sample trait data) are expressed as a plurality of disjoint continuous ranges of continuous measurement values, and categories of patient data (e.g., gene expression data and/or sample trait data) may be expressed as a plurality of disjoint sets of measurement values (e.g., {“high”, “low”}, {“high”, “normal”}, {“low”, “normal”}, {“high”, “borderline high”, “normal”, “low”}, {“Yes”, “No”}, {“Present”, “Absent”} etc.). In some embodiments, sample traits comprise clinical labels indicating the patient's health history, such as a diagnosis of a disease or disorder, a previous administering of a clinical treatment (e.g., a drug, a surgical treatment, chemotherapy, radiotherapy, immunotherapy, etc.), physical traits (age, sex, ancestry, etc.), behavioral factors, or other health status (e.g., hypertension or high blood pressure, hyperglycemia or high blood glucose, hypercholesterolemia or high blood cholesterol, history of allergic reaction or other adverse reaction, etc.).

Provided herein are computer systems programmed to implement the methods described herein. FIG. 5 shows a computer system 1101 that is programmed or otherwise configured to implement methods provided herein.

In some embodiments, the computer system 1101 is an electronic device of a user or a computer system that is remotely located with respect to the electronic device. In some embodiments, the electronic device is a mobile electronic device.

In some embodiments, the computer system 1101 comprises a central processing unit (CPU, also “processor” and “computer processor” herein) 1105, which is a single core or multi core processor, or a plurality of processors for parallel processing. In some embodiments, the computer system 1101 also comprises memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. In some embodiments, the memory 1110, storage unit 1115, interface 1120 and peripheral devices 1125 are in communication with the CPU 1105 through a communication bus (solid lines), such as a motherboard. In some embodiments, the storage unit 1115 is a data storage unit (or data repository) for storing data. In some embodiments, the computer system 1101 is operatively coupled to a computer network (“network”) 1130 with the aid of the communication interface 1120. In some embodiments, the network 1130 is the Internet, an internet and/or extranet, or an intranet and/or extranet that is in communication with the Internet.

In some embodiments, the network 1130 is a telecommunication and/or data network. In some embodiments, the network 1130 comprises one or more computer servers, which enable distributed computing, such as cloud computing. For example, in some embodiments, the one or more computer servers enable cloud computing over the network 1130 (“the cloud”) to perform various aspects of analysis, calculation, and generation of the present disclosure, such as, for example, obtaining a data set comprising gene expression data of genes of an initial gene set, from a plurality of patients; selecting N genes from the initial gene set, said N genes are N variably expressed genes of a first gene set, wherein the first gene set is a subset of the initial gene set, each gene of the first gene set can be mapped to at least one known protein, and N is an integer number; grouping the N genes into a plurality of gene modules based at least on coexpression of the N genes; correlating the plurality of gene modules with one or more sample traits, and selecting a plurality of significant gene modules based at least on strength of the correlation; overlapping one or more significant gene modules with one or more gene function signature lists; annotating the one or more significant gene modules with one or more functional characterizations based on sufficient overlap between one or more significant gene modules and the one or more gene function signature lists, wherein significant overlap satisfies overlap of a threshold minimum number of genes; and partitioning the plurality of patients into two or more treatment groups, wherein (i) all patients in a treatment group are associated with a set of significant gene modules, or (ii) each significant module of the set of significant gene modules is associated with the same functional characterization, or both. In some embodiments, the cloud computing is provided by cloud computing platforms such as, for example, Amazon Web Services (AWS), Microsoft Azure, Google Cloud Platform, and IBM cloud. In some embodiments, the network 1130 implements a peer-to-peer network, which enables devices coupled to the computer system 1101 to behave as a server. In some embodiments, the network 1130 and the computer system 1101 implements a peer-to-peer network, which enables devices coupled to the computer system 1101 to behave as a server.

In some embodiments, the CPU 1105 executes a sequence of machine readable instructions, which can be embodied in a program or software. In some embodiments, the instructions are stored in a memory location, such as the memory 1110. In some embodiments, the instructions are directed to the CPU 1105, which can subsequently program or otherwise configure the CPU 1105 to implement methods of the present disclosure. In some embodiments, examples of operations performed by the CPU 1105 comprise fetch, decode, execute, and writeback.

In some embodiments, the CPU 1105 is part of a circuit, such as an integrated circuit. In some embodiments, the circuit comprises one or more other components of the system 1101. In some embodiments, the circuit is an application specific integrated circuit (ASIC).

In some embodiments, the storage unit 1115 stores files, such as drivers, libraries and saved programs. The storage unit 1115 can store user data, e.g., user preferences and user programs. In some embodiments, the computer system 1101 comprise one or more additional data storage units that are external to the computer system 1101, such as located on a remote server that is in communication with the computer system 1101 through an intranet or the Internet.

In some embodiments, the computer system 1101 communicates with one or more remote computer systems through the network 1130. In some embodiments, the computer system 1101 communicates with a remote computer system of a user. In some embodiments, the remote computer systems comprise personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. In some embodiments, the user access the computer system 1101 via the network 1130.

In some embodiments, methods as described herein are implemented by way of machine (e.g., computer processor) executable code stored on an electronic storage location of the computer system 1101, such as, for example, on the memory 1110 or electronic storage unit 1115. In some embodiments, the machine executable or machine readable code is provided in the form of software. In some embodiments, the code is executed by the processor 1105. In some embodiments, the code is retrieved from the storage unit 1115 and stored on the memory 1110 for ready access by the processor 1105. In some embodiments, the electronic storage unit 1115 is precluded, and machine executable instructions are stored on memory 1110.

In some embodiments, the code is pre-compiled and configured for use with a machine having a processor adapted to execute the code, or is compiled during runtime. In some embodiments, the code is supplied in a programming language that is selected to enable the code to execute in a pre-compiled or as-compiled fashion.

In some embodiments, aspects of the systems and methods provided herein, such as the computer system 1101, are embodied in programming. In some embodiments, various aspects of the technology are thought of as “products” or “articles of manufacture” typically in the form of machine (or processor) executable code and/or associated data that is carried on or embodied in a type of machine readable medium. In some embodiments, machine executable code is stored on an electronic storage unit, such as memory (e.g., read-only memory, random-access memory, flash memory) or a hard disk. In some embodiments, machine executable code comprises a computer program including instructions executable by a processor to run an application. In some embodiments, a computer comprises storage media as described herein. In some embodiments, storage media comprises any or all of the tangible memory of a computer, processor, or the like, or associated modules thereof, such as various semiconductor memories, tape drives, disk drives and the like. In some embodiments, the storage media as described herein, provides non-transitory storage at any time for a computer program or a software program. In some embodiments, a computer as described herein comprises a computer-readable storage media or a non-transitory computer-readable storage media. In some embodiments, a non-transitory computer-readable storage media comprises non-volatile memory or non-volatile computer memory. In some embodiments, a non-transitory computer-readable storage media econdes a computer program including instructions executable by a processor to run an application for identifying and comparing gene expression data. In some embodiments, a non-transitory computer-readable storage media econdes a software program including instructions executable by a processor to run an application. In some embodiments, a non-transitory computer-readable storage media econdes a software program including instructions executable by a computer to run an application for identifying and/or comparing gene expression data. In some embodiments, all the software or portions of the software are, at times, communicated through the Internet or various other telecommunication networks. In some embodiments, communications through the Internet or various other telecommunication networks enable loading of the software from one computer or processor into another. In some embodiments, loading of the software as described herein comprises, for example, loading from a management server or host computer into the computer platform of an application server. In some embodiments, another type of media that bears the software elements comprises optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible storage media, terms such as computer or machine readable medium refer to any medium that participates in providing instructions to a processor for execution. In some embodiments, a computer as described herein comprises a program including instructions executable by a processor to run an application.

Hence, a machine readable medium, such as computer-executable code, may take many forms, including but not limited to, a tangible storage medium, a carrier wave medium or physical transmission medium. Non-volatile storage media comprise, for example, optical or magnetic disks, such as any of the storage devices in any computer(s) or the like, such as may be used to implement the databases, etc. shown in the drawings. Volatile storage media comprise dynamic memory, such as main memory of such a computer platform. Tangible transmission media comprise coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. In some embodiments, non-volative storage media is non-transitory storage media. In some embodiments, non-volative storage media is non-transitory computer-readable storage media. In some embodiments, volative storage media is transitory storage media. In some embodiments, volative storage media is transitory computer-readable storage media. In some embodiments a computer as described herein comprises non-volative storage media and volatile storage media. In some embodiments, a computer as described herein comprises non-transitory storage media and transitory storage media. In some embodiments, a computer as described herein comprises non-transitory storage media encoded with a computer program or a software comprising instructions executable by a processor to run an application. As described herein, storage media is computer-readable media. Common forms of computer-readable media therefore comprise for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer-readable media may be involved in carrying one or more sequences of one or more instructions to a processor for execution.

In some embodiments, the computer system 1101 comprises or is in communication with an electronic display 1135 that comprises a user interface (UI) 1140. Examples of user interfaces (UIs) comprise, without limitation, a graphical user interface (GUI) and web-based user interface. For example, the computer system can comprise a graphical user interface (GUI) configured to display, for example, patient data, identification of a lung nodule of the patient as a malignant lung nodule or a benign lung nodule, and/or predictions or assessments generated from patient data.

In some embodiments, the methods and systems provided herein are implemented by way of one or more algorithms. In some embodiments, the algorithm is implemented by way of software upon execution by the central processing unit 1105. In some embodiments, the algorithm, for example, obtains or assesses a data set comprising gene expression data of genes of an initial gene set, from a plurality of patients; select N genes from the initial gene set, said N genes are N variably expressed genes of a first gene set, wherein the first gene set is a subset of the initial gene set, each gene of the first gene set can be mapped to at least one known protein, and N is an integer number; group the N genes into a plurality of gene modules based at least on coexpression of the N genes; correlate the plurality of gene modules with one or more sample traits, and selecting a plurality of significant gene modules based at least on strength of the correlation; overlap one or more significant gene modules with one or more gene function signature lists; annotate the one or more significant gene modules with one or more functional characterizations based on sufficient overlap between one or more significant gene modules and the one or more gene function signature lists, wherein significant overlap satisfies overlap of a threshold minimum number of genes; and partition the plurality of patients into two or more treatment groups, wherein (i) all patients in a treatment group are associated with a set of significant gene modules, or (ii) each significant module of the set of significant gene modules is associated with the same functional characterization, or both.

### IV. Methods of Comprising Different Patient Populations

In some embodiments, the methods described herein comprise different patient populations, different patient treatment groups, and/or different patient subsets. In some embodiments, the methods described herein comprise a patient population comprising subsets of patients. In some embodiments, a patient subset is a patient cluster as described herein.

In some embodiments, different patient populations, different patient treatment groups, and/or different patient subsets are determined in consideration of gene expression data corresponding to a disease or disorder as described herein. In some embodiments, different patient populations, different patient treatment groups, and/or different patient subsets are determined in consideration of a gene module corresponding to a disease or disorder as described herein. In some embodiments, different patient populations, different patient treatment groups, and/or different patient subsets are determined in consideration of a progression of a disease or disorder. In some embodiments, analysis of gene expression data from a patient sample determines the patient population, treatment group, and/or patient subset for a patient with a disease or disorder as described herein. In some embodiments, analysis of gene expression data from a patient sample as compared to reference gene modules and sample traits determines the patient population, treatment group, and/or patient subset for a patient with a disease or disorder as described herein.

In some embodiments, different patient populations, different patient treatment groups, and/or different patient subsets are determined in consideration of at least one gene set variation analysis (GSVA). In some embodiments, two or more patient populations, two or more patient treatment groups, and/or two or more patient subsets are determined in consideration of at least one gene set variation analysis (GSVA). In some embodiments, GSVA scores are generated using the gene modules as input for GSVA. In some embodiments, GSVA scores are generated using the significant gene modules as input for GSVA. In some embodiments, GSVA scores are generated using the plurality of significant gene modules as input for GSVA. In some embodiments, GSVA scores are generated as described herein, and/or as understood by one of ordinary skill in the art. In some embodiments, different patient populations, different patient treatment groups, and/or different patient subsets are determined in consideration of GSVA scores and k-means clustering method. In some embodiments, different patient populations, different patient treatment groups, and/or different patient subsets are determined in consideration of module eigengenes (MEs) of the significant gene modules as described herein. In some embodiments, MEs are calculated using the gene modules as input. In some embodiments, MEs are calculated using the significant gene modules as input. In some embodiments, MEs are calculated using the plurality of gene modules as input. In some embodiments, MEs are calculated as described herein, and/or as understood by one of ordinary skill in the art. In some embodiments, different patient populations, different patient treatment groups, and/or different patient subsets are determined in consideration of MEs and k-means clustering method.

In some embodiments, different patient populations, different patient treatment groups, and/or different patient subsets are determined in consideration of a machine learning model as described herein. In some embodiments, different patient populations, different patient treatment groups, and/or different patient subsets are determined in consideration of a trained machine learning model as described herein. In some embodiments, the machine learning model is trained using the gene expression data as input. In some embodiments, the machine learning model is trained using the gene modules as input. In some embodiments, the machine learning model is trained using the significant gene modules as input. In some embodiments, the machine learning model is trained using the plurality of gene modules as input. In some embodiments, the machine learning model is trained as described herein, and/or as understood by one of ordinary skill in the art.

In some embodiments, different patient populations, different patient treatment groups, and/or different patient subsets are determined to be a first patient population, a second patient population, a third patient population, etc.

In some embodiments, the methods described herein comprise methods of treatment of a disease or disorder as described herein. In some embodiments, the methods of treatment of a disease or disorder comprise treatment of patients in a first patient population, and treatment of patients in a second patient population. In some embodiments, the treatment of patients in a first patient population is different from the treatment of patients in a second patient population. In some embodiments, the methods of treatment of a disease or disorder comprise treatment of a first patient in a first patient population, and a second patient in a first patient population. In some embodiments, the treatment of a first patient in a first patient population is different from the treatment of a second patient in a first patient population. In some embodiments, the molecular endotype of a first patient in a first patient population is different from the molecular endotype of a second patient in a first patient population.

In some embodiments, the methods of treatment of a disease or disorder as described herein comprise treatment of different treatment groups. In some embodiments, the methods of treatment of a disease or disorder as described herein comprise treatment of more than one treatment group. In some embodiments, a treatment group is also referred to as a patient population.

In some embodiments, a first patient population has a disease or disorder described herein, and a second patient population is a healthy control. In some embodiments, a first patient population has a disease or disorder described herein, and a second patient population is a healthy control; and the one or more sample traits comprise one or more sample traits listed in TABLE 6. In some embodiments, a first patient population has a disease or disorder described herein, and a second patient population is a healthy control; and the one or more gene function signature lists comprise one or more gene function signature lists in TABLE 7.

In some embodiments, a first patient population has a disease or disorder described herein, and a second patient population has a different disease or disorder described herein. In some embodiments, a first patient population has a disease or disorder described herein, and a second patient population has a different disease or disorder described herein; and the one or more sample traits comprise one or more sample traits listed in TABLE 6. In some embodiments, a first patient population has a disease or disorder described herein, and a second patient population has a different disease or disorder described herein; and the one or more gene function signature lists comprise one or more gene function signature lists in TABLE 7.

In some embodiments, a first patient population has a first disease or disorder described herein, and a second patient population has a second disease or disorder described herein. In some embodiments, a first patient population has a first disease or disorder described herein, and a second patient population has a second disease or disorder described herein; and the one or more sample traits comprise one or more sample traits listed in TABLE 6. In some embodiments, a first patient population has a first disease or disorder described herein, and a second patient population has a second disease or disorder described herein; and the one or more gene function signature lists comprise one or more gene function signature lists in TABLE 7.

In some embodiments, a first patient population has a chronic condition, inflammatory condition, and/or autoimmune condition, and a second patient population has a different chronic condition, inflammatory condition, and/or autoimmune condition. In some embodiments, a first patient population has a chronic condition, inflammatory condition, and/or autoimmune condition, and a second patient population has a different chronic condition, inflammatory condition, and/or autoimmune condition; and the one or more sample traits comprise one or more sample traits listed in TABLE 6. In some embodiments, a first patient population has a chronic condition, inflammatory condition, and/or autoimmune condition, and a second patient population has a different chronic condition, inflammatory condition, and/or autoimmune condition; and the one or more gene function signature lists comprise one or more gene function signature lists in TABLE 7.

In some embodiments, a first patient population has a chronic condition, and a second patient population has a different chronic condition. In some embodiments, a first patient population has a chronic condition, and a second patient population has a different chronic condition; and the one or more sample traits comprise one or more sample traits listed in TABLE 6. In some embodiments, a first patient population has a chronic condition, and a second patient population has a different chronic condition; and the one or more gene function signature lists comprise one or more gene function signature lists in TABLE 7.

In some embodiments, a first patient population has an inflammatory condition, and a second patient population has a different inflammatory condition. In some embodiments, a first patient population has an inflammatory condition, and a second patient population has a different inflammatory condition; and the one or more sample traits comprise one or more sample traits listed in TABLE 6. In some embodiments, a first patient population has an inflammatory condition, and a second patient population has a different inflammatory condition; and the one or more gene function signature lists comprise one or more gene function signature lists in TABLE 7.

In some embodiments, a first patient population has an autoimmune condition, and a second patient population has a different autoimmune condition. In some embodiments, a first patient population has an autoimmune condition, and a second patient population has a different autoimmune condition; and the one or more sample traits comprise one or more sample traits listed in TABLE 6. In some embodiments, a first patient population has an autoimmune condition, and a second patient population has a different autoimmune condition; and the one or more gene function signature lists comprise one or more gene function signature lists in TABLE 7.

In some embodiments, a first patient population has an arthritis, and a second patient population has a different arthritis. In some embodiments, a first patient population has an arthritis, and a second patient population has a different arthritis; and the one or more sample traits comprise one or more sample traits listed in TABLE 6. In some embodiments, a first patient population has an arthritis, and a second patient population has a different arthritis; and the one or more gene function signature lists comprise one or more gene function signature lists in TABLE 7.

In some embodiments, a first patient population has an inflammatory arthritis, and a second patient population has a non-inflammatory arthritis. In some embodiments, a first patient population has an inflammatory arthritis, and a second patient population has a non-inflammatory arthritis; and the one or more sample traits comprise one or more sample traits listed in TABLE 6. In some embodiments, a first patient population has an inflammatory arthritis, and a second patient population has a non-inflammatory arthritis; and the one or more gene function signature lists comprise one or more gene function signature lists in TABLE 7.

In some embodiments, a first patient population has a non-inflammatory arthritis, and a second patient population has an inflammatory arthritis. In some embodiments, a first patient population has a non-inflammatory arthritis, and a second patient population has an inflammatory arthritis; and the one or more sample traits comprise one or more sample traits listed in TABLE 6. In some embodiments, a first patient population has a non-inflammatory arthritis, and a second patient population has an inflammatory arthritis; and the one or more gene function signature lists comprise one or more gene function signature lists in TABLE 7.

In some embodiments, a first patient population has early inflammatory arthritis, and a second patient population has advanced Rheumatoid arthritis. In some embodiments, a first patient population has early inflammatory arthritis, and a second patient population has advanced Rheumatoid arthritis; and the one or more sample traits comprise one or more sample traits listed in TABLE 6. In some embodiments, a first patient population has early inflammatory arthritis, and a second patient population has advanced Rheumatoid arthritis; and the one or more gene function signature lists comprise one or more gene function signature lists in TABLE 7.

In some embodiments, the one or more gene function signature lists are selected from the one or more gene function signature lists in TABLE 7.

In some embodiments, the methods described herein comprise a patient population comprising at least two subsets of patients. In some embodiments, a patient subset is a patient cluster as described herein. In some embodiments, the methods described herein comprise a first patient population comprising at least two subsets of patients. In some embodiments, the methods described herein comprise a second patient population comprising at least two subsets of patients. In some embodiments, the methods described herein comprise a third patient population comprising at least two subsets of patients.

In some embodiments, a first patient population comprises different patient subsets, and/or different patient clusters. In some embodiments, a first patient population comprises different patient treatment groups. In some embodiments, a patient subset corresponds to a patient treatment group. In some embodiments, a patient cluster corresponds to a patient treatment group.

In some embodiments, a patient subset, as detailed in FIG. 7, FIG. 8, FIG. 9, FIG. 10, FIG. 11, or FIG. 12, corresponds to a disease phenotype of a disease or disorder described herein. In some embodiments, a patient cluster, as detailed in FIG. 7, FIG. 8, FIG. 9, FIG. 10, FIG. 11, or FIG. 12, corresponds to disease phenotype of a disease or disorder described herein. In some embodiments, a patient subset, as detailed in FIG. 7, FIG. 8, FIG. 9, FIG. 10, FIG. 11, or FIG. 12, corresponds to a patient treatment group. In some embodiments, a patient cluster, as detailed in FIG. 7, FIG. 8, FIG. 9, FIG. 10, FIG. 11, or FIG. 12, corresponds to a patient treatment group.

In some embodiments, each of the at least two subsets of patients corresponding to a different disease phenotype of a disease or disorder described herein. In some embodiments, each of the at least two subsets of patients corresponding to a different disease phenotype of an established disease or disorder.

In some embodiments, patient subsets or patient clusters are determined using idealized k-means clustering as described herein. In some embodiments, a patient subset or a patient cluster determined using idealized k-means clustering corresponds to a disease phenotype. In some embodiments, a patient subset or a patient cluster determined using idealized k-means clustering corresponds to treatment group. In some embodiments, a patient subset or a patient cluster determined using idealized k-means clustering corresponds to a clinical outcome. In some embodiments, a patient subset or a patient cluster, as detailed in FIG. 7, FIG. 8, FIG. 9, FIG. 10, FIG. 11, or FIG. 12, is determined using idealized k-means clustering. In some embodiments, stable k-means clustering revealed groupings of clinical traits and correlated molecular functions. In some embodiments, patient subsets or patient clusters are determined using a machine learning model described herein.

In some embodiments, different patient populations, different patient treatment groups, and/or different patient subsets are determined in consideration of gene expression data from a patient. In some embodiments, different patient populations, different patient treatment groups, and/or different patient subsets are determined in consideration of gene expression data from a plurality of patients. In some embodiments, different patient populations, different patient treatment groups, and/or different patient subsets are determined in consideration of gene expression data from at least 2 genes of a plurality of significant gene modules associated with a disease or disorder.

In some embodiments, the first patient population comprises a first patient, a second patient, a third patient, etc. In some embodiments, a first patient is determined to have a different molecular endotype from a second patient. In some embodiments, a first patient is determined to have a different molecular endotype from a third patient. In some embodiments, a second patient is determined to have a different molecular endotype from a third patient. In some embodiments, a first patient population comprises one or more molecular endotypes and/or one or more disease phenotypes, corresponding to one or more treatment groups. In some embodiments, the first patient population comprises one or more patient subsets. In some embodiments, the first patient population comprises patient subsets in consideration of the gene expression data as it relates to molecular endotype, disease phenotype, and treatment group. In some embodiments, the first patient population comprises one or more patient clusters. In some embodiments, the first patient population comprises patient clusters in consideration of the gene expression data as it relates to molecular endotype, disease phenotype, and treatment group.

In some embodiments, the first patient population to the gene expression data of the second patient in the first patient population is used to identify genetic biomarkers that correlate to a clinical profile of a patient or a clinical outcome of a disease or disorder of a patient. In some embodiments, the method for predicting a clinical outcome of a disease or disorder of a patient comprises more than two patients in a first patient population, more than two isolated biological samples, and/or more than two different disease phenotypes.

In some embodiments, the first patient population comprises treatment-naïve patients. In some embodiments, treatment-naïve patients are patients that have not received a treatment as described herein for a disease or disorder as described herein. In some embodiments, the first patient population comprises DMARD-naïve patients. In some embodiments, DMARD-naïve patients are patients that have not received a DMARD treatment as described herein for a disease or disorder as described herein. In some embodiments, the first patient population comprises TFi-naïve patients. In some embodiments, TFi-naïve patients are patients that have not received a TFi treatment as described herein for a disease or disorder as described herein. In some embodiments, the first patient population comprises biologic naïve patients. In some embodiments, biologic naïve patients are patients that have not received a biologic treatment as described herein for a disease or disorder as described herein. In some embodiments, the first patient population comprises incomplete responders (IRs). In some embodiments, the first patient population comprises DMARD incomplete responders (DMARD IR), or IR to DMARD treatment. In some embodiments, the first patient population comprises TNF inhibitor incomplete responders (TNFi IR), or IR to TNFi treatment. In some embodiments, the first patient population comprises biologic treatment incomplete responders (biologic IR), or IR to biologic treatment. In some embodiments, the method identifies more than one disease or disorder. In some embodiments, the method identifies a first disease or disorder, a second disease or disorder, and/or a third disease or disorder. In some embodiments, the first disease or disorder is different from the second disease or disorder, the first disease or disorder is different from the third disease or disorder, and the second disease or disorder is different from the third disease or disorder.

In some embodiments, a patient population associated with a disease or disorder as described herein is partitioned into the two or more treatment groups based at least on training a machine learning model to infer a treatment group for a reference patient. In some embodiments, the machine learning model is trained to infer a treatment group for a reference patient based on i) gene expressions of at least 2 genes of the plurality of significant gene modules, in a reference biological sample from the reference patient, and/or ii) the reference patient's one or more sample traits. In some embodiments, the machine learning model is trained to infer a treatment group for a reference patient based on GSVA scores of the reference patient. In some embodiments, the machine learning model is trained to infer a treatment group for a reference patient based on MEs of the reference patient. The GSVA scores and/or MEs of a reference patient can be calculated as described herein. In some embodiments, the machine learning model is trained to infer a treatment group for a reference patient based on i) gene expressions of at least 2 genes of the plurality of significant gene modules, in a reference biological sample from the reference patient, and ii) the reference patient's one or more sample traits.

In some embodiments, a patient population associated with a disease or disorder as described herein is partitioned into the two or more treatment groups based at least on a machine learning model trained to infer a treatment group for a patient based on the patient's one or more sample traits. In some embodiments, the machine learning model infers a treatment group for a patient based on i) gene expression data of at least 2 genes of the plurality of significant gene modules in an isolated biological sample from the patient. In some embodiments, the patient population is partitioned into the two or more treatment groups based on gene expression data of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 550, 600, 650, 700, 750, 800, 850, 900, 950, 1000, 1100, 1200, 1300, 1400, 1500, 1600, 1700, 1800, 1900, or 2000 genes of the plurality of significant gene modules in an isolated biological sample from the patient. In some embodiments, the patient population is partitioned into the two or more treatment groups based on gene expression data and/or at least 1, 2, 3, 4, 5, 6, 7, 8, 9, or 10 sample traits.

In some embodiments, the methods described herein comprise determining treatment methods for the two or more treatment groups. In some embodiments, a treatment method for a treatment group is determined in consideration of at least the functional annotation of the one or more significant gene modules associated with the treatment group. In some embodiments, the methods described herein comprise determining treatment methods for the two or more treatment groups in consideration of the progression of the disease or disorder.

In some embodiments, the progression of the disease or disorder determines the methods for treating a disease or disorder in a patient. In some embodiments, the progression of the disease or disorder determines the methods for treating, preventing, or inhibiting a disease or disorder as described herein. In some embodiments, the progression of the disease or disorder shifts clinical outcomes.

### V. Methods for Predicting a Clinical Outcome of a Disease or Disorder of a Patient

Described herein are methods for predicting a clinical outcome of a disease or disorder in a patient.

Also provided herein are methods described herein comprising assays for the generation of gene expression data from the sample from the patient, for accurate, repeatable, real-time, determinations of a disease or disorder in a patient (e.g., classifying a disease or disorder, determining progression of disease or disorder, predicting a clinical outcome of a disease or disorder), useful in developing targeted personalized treatment for patients with chronic, inflammatory, and/or autoimmune diseases as described herein.

In some embodiments, methods for predicting a clinical outcome of a disease or disorder in a patient comprise at least two subsets of patients, each of the at least two subsets of patients corresponding to a different disease phenotype of an established disease or disorder.

In some embodiment, the patient is at elevated risk of having disease or disorder. In some embodiment, the patient is suspected of having disease or disorder. In some embodiment, the patient is asymptomatic for disease or disorder. In some embodiment, the patient has disease or disorder. In some embodiment, the patient is at elevated risk of having of having inactive disease or disorder. In some embodiment, the patient is suspected of having inactive disease or disorder. In some embodiment, the patient is asymptomatic for inactive disease or disorder. In some embodiment, the patient has inactive disease or disorder. In some embodiment, the patient is at elevated risk of having of having active disease or disorder. In some embodiment, the patient is suspected of having active disease or disorder. In some embodiment, the patient is asymptomatic for active disease or disorder. In some embodiment, the patient has active disease or disorder. In some embodiments, the disease or disorder as described herein is any type of disease or disorder as described herein.

In some embodiment, the patient is at elevated risk of having more than one disease or disorder. In some embodiment, the patient is suspected of having more than one disease or disorder. In some embodiment, the patient is asymptomatic for more than one disease or disorder. In some embodiment, the patient has more than one disease or disorder. In some embodiment, the patient is at elevated risk of having of having inactive more than one disease or disorder. In some embodiment, the patient is suspected of having inactive more than one disease or disorder. In some embodiment, the patient is asymptomatic for inactive more than one disease or disorder. In some embodiment, the patient has inactive more than one disease or disorder. In some embodiment, the patient is at elevated risk of having of having active more than one disease or disorder. In some embodiment, the patient is suspected of having active more than one disease or disorder. In some embodiment, the patient is asymptomatic for active more than one disease or disorder. In some embodiment, the patient has active more than one disease or disorder. In some embodiments, the more than one disease or disorder as described herein is any type of more than one disease or disorder as described herein.

In some embodiment, the patient is at elevated risk of having arthritis. In some embodiment, the patient is suspected of having arthritis. In some embodiment, the patient is asymptomatic for arthritis. In some embodiment, the patient has arthritis. In some embodiment, the patient is at elevated risk of having of having inactive arthritis. In some embodiment, the patient is suspected of having inactive arthritis. In some embodiment, the patient is asymptomatic for inactive arthritis. In some embodiment, the patient has inactive arthritis. In some embodiment, the patient is at elevated risk of having of having active arthritis. In some embodiment, the patient is suspected of having active arthritis. In some embodiment, the patient is asymptomatic for active arthritis. In some embodiment, the patient has active arthritis. In some embodiments, the disease or disorder as described herein is any type of arthritis as described herein. In some embodiments, the arthritis is selected from: a chronic condition, an inflammatory condition, an autoimmune condition, an arthritis, a rheumatoid arthritis (RA), an early inflammatory arthritis (EIA), an inflammatory arthritis, a psoriatic arthritis (PSA), a lupus arthritis, a rhupus, an osteoarthritis, a non-inflammatory arthritis, a pauci-inflammatory arthritis, or combinations thereof. In some embodiment, the patient is at elevated risk of having more than one arthritis. In some embodiment, the patient is suspected of having more than one arthritis. In some embodiment, the patient is asymptomatic for more than one arthritis. In some embodiment, the patient has more than one arthritis. In some embodiments, the patient has and/or experiencing more than one arthritis.

### VI. Methods of Treating a Disease or Disorder

Described herein are methods for treating a disease or disorder in a patient.

In some embodiments, methods comprise treating, preventing, or inhibiting a disease or disorder as described herein. In some embodiments, methods comprise treating, preventing, or inhibiting a disease or disorder associated with a gene module. In some embodiments, methods comprise treating, preventing, or inhibiting a disease or disorder associated with one or more gene modules. In some embodiments, methods comprise treating, preventing, or inhibiting a disease or disorder associated with a plurality of gene modules. In some embodiments, methods for treating a disease or disorder comprise methods of identifying a disease or disorder as described herein. In some embodiments, methods for treating a disease or disorder comprise methods of identifying a disease or disorder or a susceptibility thereof of the patient as described herein. In some embodiments, compositions, systems, or kits described herein are for use in a method for treating a disease or disorder. In some embodiments, compositions, systems, or kits described herein are for use in the manufacture of a medicament for treating a disease or disorder. In some embodiments, compositions, systems, or kits described herein are for the administration of a drug for treating a disease or disorder as described herein. In some embodiments, compositions, systems, or kits described herein are for the administration of a composition for the treatment of a disease or disorder as described herein. In some embodiments, the composition is a pharmaceutical composition.

In some embodiments, the method for treating a disease or disorder comprises identifying the treatment the patient with a disease or disorder is likely to respond to. In some embodiments, the method for treating a disease or disorder comprises the identification of treatment effectiveness and, if necessary, treatment adjustment. In some embodiments, the method for treating a disease or disorder comprises identifying the effectiveness of the treatment as compared to a progression of a disease or disorder in a patient. In some embodiments, the method for treating a disease or disorder comprises the administration of a drug as described herein.

In some embodiments, the method for treating a disease or disorder comprises treatment adjustment in consideration of a method for identifying a report as described herein. In some embodiments, methods described herein comprise electronically outputting a report detailing the comparison of (i) the gene expression data set generated from assaying the isolated biological sample to (ii) the reference gene expression data set from one or more gene modules. In some embodiments, the report is output of a computer comprising a non-transitory computer-readable storage media encoded with a computer program including instructions executable by a processor to run an application. In some embodiments, the report (i) identifies an immunological state of the patient at an accuracy of at least about 70%; (ii) identifies a disease or disorder or a susceptibility thereof of the patient at an accuracy of at least about 70%; (iii) identifies if the patient is likely to respond to a treatment comprising administration of a drug selected from: a immunoregulator, a immunosuppressant, a steroid, an anti-inflammatory, a JAK inhibitors, a TNF inhibitors, a baricitinib, a corticosteroid, a nonsteroidal anti-inflammatory drug (NSAID), a tofacitinib, a upadacitinib, a deucravacitinib, a brepocitinib, a disease-modifying anti-rheumatic drug (DMARD), a conventional synthetic DMARD (csDMARD), a targeted synthetic DMARD (tsDMARD), a biologic DMARD (bDMARD), a biologic treatment, a TYK2 inhibitor, a TYK2/JAK inhibitor, a combination inhibitor, a monoclonal antibody, an anti-TNF biologic, anti-IL-6 biologic, anti-IL-17 biologic, anti-IL-12/23 biologic, and anti-CD28 biologic, or combinations thereof; and/or (v) identifies an effectiveness of the treatment of the patient as compared to the progression of a disease or disorder.

In some embodiments, the report identifies an immunological state of the patient at an accuracy of at least about 50%, at least about 55%, at least about 60%, at least about 65%, at least about 70%, at least about 75%, at least about 80%, at least about 85%, at least about 90%, at least about 95%, or at least about 100%.

In some embodiments, the report identifies a disease or disorder or a susceptibility thereof of the patient at an accuracy of at least about 50%, at least about 55%, at least about 60%, at least about 65%, at least about 70%, at least about 75%, at least about 80%, at least about 85%, at least about 90%, at least about 95%, or at least about 100%.

In some embodiments, the report identifies if the patient is likely to respond to a treatment comprising administration of a drug described herein. In some embodiments, the report identifies if the patient is likely to respond to a treatment comprising administration of a drug directed for the treatment of a disease or disorder as described herein.

In some embodiments, the report identifies an effectiveness of the treatment of the patient as compared to the progression of a disease or disorder. In some embodiments, the report identifies the effectiveness of the treatment as described herein. In some embodiments, the report identifies the progression of a disease or disorder.

In some embodiments, treating, preventing, or inhibiting disease or disorder in a patient comprises any of the methods described herein. In some embodiments, the methods of treating, preventing, or inhibiting a disease or disorder in a patient involves identification of a phenotype. In some embodiments, the phenotype is associated with a disease or disorder, an organ involvement, a medication response, a treatment response, a treatment target, a molecular endotype, or any combination thereof. In some embodiments, the methods of treating, preventing, or inhibiting a disease or disorder in a patient involve the identification of a molecular endotype of a disease or disorder. In some embodiments, methods of treating, preventing, or inhibiting disease or disorder in a patient comprises administration of a drug described herein. In some embodiments, methods of treating, preventing, or inhibiting disease or disorder in a patient comprises administration of a treatment to the patient described herein. In some embodiments, methods of treating, preventing, or inhibiting disease or disorder in a patient comprises parenteral administration of a drug to the patient. In some embodiments, methods of treating, preventing, or inhibiting disease or disorder in a patient comprise administration for at least zero weeks, 16 weeks, and 52 weeks, at least 1 year, at least 2 years, at least 3 years, at least 4 years, at least 5 years, at least 6 years, at least 7 years, at least 8 years, at least 9 years, 10 years, at least 15 years, at least 20 years, at least 30 years, at least 35 years, at least 40 years, at least 45 years, at least 50 years, or at least the patient lifespan. In some embodiments, the treatment is adjusted as a function of the gene expression data. In some embodiments, the gene expression data is used to identify a drug for the treatment of the disease or disorder. In some embodiments, the rate of treatment administration is adjusted as a function of the drug for the treatment of the disease or disorder. In some embodiments, the rate of treatment administration is adjusted as a function of the disease or disorder in a patient. In some embodiments, the rate of treatment administration is adjusted as a function of the progression of the disease or disorder.

Also described herein are methods for treating a disease or disorder in a patient, the methods comprising predicting a clinical outcome of the disease or disorder. In some embodiments, the clinical outcome is associated to the progression of the disease or disorder. In some embodiments, the clinical outcome is associated to preventing a disease or disorder. In some embodiments, the clinical outcome is associated to inhibiting the disease or disorder. In some embodiments, the methods of treating, preventing, or inhibiting a disease or disorder in a patient involves identification of a phenotype.

In some embodiments, treating, preventing, or inhibiting disease or disorder in a patient comprises methods for predicting a clinical outcome as described herein. In some embodiments, the clinical outcome comprises a treatment. In some embodiments, the treatment comprises administration of a drug for the treatment of a disease or disorder in a patient. In some embodiments, the treatment comprises administration of a drug to the patient as described herein. In some embodiments, the treatment comprises parenteral administration of a drug to the patient as described herein. In some embodiments, the treatment is adjusted as a function of the gene expression data generated from assaying an isolated biological sample from a patient. In some embodiments, the treatment is adjusted as a function of the gene expression data. In some embodiments, the gene expression data is used to identify a drug for a treatment of a disease or disorder. In some embodiments, the drug is selected from the group consisting of: an immunoregulator, an immunosuppressant, a steroid, an anti-inflammatory, a JAK inhibitor, a TNF inhibitor, a baricitinib, a corticosteroid, a nonsteroidal anti-inflammatory drug (NSAID), a tofacitinib, a upadacitinib, a deucravacitinib, a brepocitinib, a disease-modifying antirheumatic drug (DMARD), a conventional synthetic DMARD (csDMARD), a targeted synthetic DMARD (tsDMARD), a biologic DMARD (bDMARD), a biologic treatment, a TYK2 inhibitor, a TYK2/JAK inhibitor, a combination inhibitor, a monoclonal antibody, an anti-TNF biologic, anti-IL-6 biologic, anti-IL-17 biologic, anti-IL-12/23 biologic, and anti-CD28 biologic, and any combination thereof.

In some embodiments, the method for predicting the clinical outcome of the disease or disorder of the patient comprises defining a signature that is predictive of transcript levels that indicate the clinical outcome of the disease or disorder of the patient. In some embodiments, defining a signature as described herein comprises a comparison of the data set comprising the gene expression data of a first patient in a first patient population to the gene expression data of a second patient in the first patient population. In some embodiments, the method for predicting the clinical outcome of the disease or disorder of the patient comprises electronically outputting a report detailing the signature as described herein. In some embodiments, the signature is: (i) a transcriptomic signature; (ii) predictive of clinical outcomes comprising a treatment of a disease or disorder; or (iii) predictive of clinical outcomes of a disease or disorder of a patient from gene expression data and clinical data. In some embodiments, the signature is: (i) a transcriptomic signature; (ii) predictive of clinical outcomes comprising a treatment of a disease or disorder; or (iii) predictive of clinical outcomes of a disease or disorder of a patient from gene expression data and clinical data, as detailed in FIG. 7, FIG. 8, FIG. 9, FIG. 10, FIG. 11, or FIG. 12. In some embodiments, the method for predicting the clinical outcome of the disease or disorder comprises assaying samples from at least two subsets of patients, with each subset corresponding to a different disease phenotype of an established disease or disorder. In some embodiments, the first patient in the first patient population comprises a different disease phenotype than the second patient in the first patient population. In some embodiments, the different disease phenotypes respond to a different treatment. In some embodiments, the different disease phenotypes correlate to a different treatment group. In some embodiments, the report comprises data used to define a phenotype. In some embodiments, the phenotype comprises a disease or disorder, an organ involvement, a medication response, or any combination thereof.

In some embodiments, the methods, systems, devices and compositions described herein are used for treating, preventing, or inhibiting a disease or disorder in a patient. In some embodiments, the disease or disorder is a chronic condition, a chronic condition, an inflammatory condition, an autoimmune condition, an arthritis, a rheumatoid arthritis (RA), an early inflammatory arthritis (EIA), an inflammatory arthritis, a psoriatic arthritis (PSA), a lupus arthritis, a rhupus, an osteoarthritis, a non-inflammatory arthritis, a pauci-inflammatory arthritis, or combinations thereof. Exemplary diseases and syndromes comprise but are not limited to the diseases and syndromes listed in the section below.

In some embodiments, methods of treatment described herein are associated to at least one treatment target associated with a disease or disorder described herein. In some embodiments, the disease or disorder comprises a disease or disorder selected from the section below.

In some embodiments, the method comprises selecting, recommending and/or administering a treatment to the patient based at least in part on the classification of the disease or disorder of the patient. In some embodiments, the method comprises administering a treatment to the patient based at least in part on the classification of the disease or disorder of the patient. In some embodiments, the method comprises selecting a treatment for the patient based at least in part on the classification of the disease or disorder of the patient. In some embodiments, the method comprises recommending a treatment to the patient based at least in part on the classification of the disease or disorder of the patient. In some embodiments, the treatment for disease or disorder is configured to treat, reduce a severity of, and/or reduce a risk of having the disease or disorder. In some embodiments, the treatment for disease or disorder comprises a drug targeting one or more genes in a gene module correlated with the disease or disorder. In some embodiments, the treatment for disease or disorder is comprises one or more treatment for the disease or disorder. In some embodiments, the treatment for the disease or disorder comprises a drug targeting one or more genes in a gene module In some embodiments, the drug targeting one or more genes in a gene module (e.g., a significant gene module, a gene module from TABLE 2, TABLE 3, TABLE 4, or TABLE 5) enriched in the sample. In some embodiments, the drug targeting one or more genes in a gene module (e.g., a significant gene module, a gene module from TABLE 2, TABLE 3, TABLE 4, or TABLE 5) enriched in an isolated biological sample from a patient. In some embodiments, the treatment comprises pharmaceutical composition.

In some embodiments, the treatment of a disease or disorder comprises the administration of a drug directed to the treatment targets of the disease or disorder, as appropriate. In some embodiment, the treatment of a disease or disorder comprises the administration of a drug selected from: a immunoregulator, a immunosuppressant, a steroid, an anti-inflammatory, a JAK inhibitors, a TNF inhibitors, a baricitinib, a corticosteroid, a nonsteroidal anti-inflammatory drug (NSAID), a tofacitinib, a upadacitinib, a deucravacitinib, a brepocitinib, a biologic disease-modifying anti-rheumatic drug (DMARD), a conventional synthetic DMARD (csDMARD), a targeted synthetic DMARD (tsDMARD), a biologic DMARD (bDMARD), a biologic treatment, a TYK2 inhibitor, a TYK2/JAK inhibitor, a combination inhibitor, a monoclonal antibody, an anti-TNF biologic, anti-IL-6 biologic, anti-IL-17 biologic, anti-IL-12/23 biologic, and anti-CD28 biologic, or combinations thereof.

In some embodiments, the treatment for the disease or disorder comprises the administration of a drug selected from: an IFN inhibitor, a neutrophil function inhibitor, a monocyte inhibitor, an IL-1 inhibitor, an TNF inhibitor, T cell inhibitor, a cell cycle inhibitor, a neurotransmitter uptake inhibitor, a neurotransmitter uptake inhibitor, B cell inhibitor, a plasma cell inhibitor, an Ig chains inhibitor, neuromuscular pathways inhibitor, or any combination thereof. In some embodiments, the treatment for the disease or disorder comprises anifrolumab, deucravacitinib, adalimumab, certolizumab pegol, etanercept, golimumab, inflximab. palbociclib, ribociclib, abemaciclib, Anakinra, Canakinumab, Dasatinib, Apremilast, Roflumilast, belimumab, rituximab, obinutuzmab, ineilizumab, ocrelizumab, ofatumumab, Mycophenolate, Bortezomib, Carfilzomib, Ixazomib, Daratumumab, Isatuximab, Elotuzumab, or combinations thereof.

In some embodiments, the treatment of the disease or disorder comprises AG-879, Aloisine, Alvocidib, Aminopurvalanol A, Amiodarone, Amiselimod, Amrinone, Arachidonyltrifluoromethane, Arcyriaflavin A, Arsenic Trioxide, AT-7519, Atorvastatin, Axitinib, Batimastat, Bisindolylmaleimide, Bortezomib, Briciclib, Cabozantinib, Cediranib, Cenerimod, Chlorpromazine, Cinnarizine, Cyclosporin A, Doxycycline, Entrectinib, Felodipine, Fingolimod, Flunarizine, GW-441756, HNHA, Ibudilast, Ilomastat, Lavendustin A, Lenvatinib, Lestaurtinib, Linifanib, Mepacrine, Mibefradil, Milrinone, Mocravimod, Nifedipine, Nimesulide, Nitrendipine, Nomifensine, Oxindole-I, Ozanimod, Palbociclib, Pazopanib, PHA-793887, Purvalanol A, Ramucirumab, Ribociclib, RO-3306, Roscovitine, Simvastatin, Siponimod, Sirolimus, Sorafenib, SSR-69071, Sunitinib, Tacrolimus, Tamoxifen, Tivozanib, Trequinsin, Vandetanib, Zardaverine, Gabexate, Omalizumab, Pepstatin, PHCCC, Cediranib, ENMD-2076, GW-501516, Linifanib, Quizartinib, Roscovitine, Acetyl-Farnesyl-Cysteine, Aminopurvalanol A, Diclofenac, Midostaurin, Palbociclib, Rosiglitazone, Sirolimus, Sorafenib, Sunitinib, Lenvatinib, Venetoclax, Pexidartinib, Pioglitazone, Lestaurtinib, Aloisine, RO-3306, TCS-359, Dorsomorphin, GSK-0660, GTP-14564, GW-1929, JTE-013, Purvalanol A, T-0070907, Troglitazone, HLI-373, JNJ-26854165, Linifanib, NUTLIN-3, Serdemetan, Axitinib, Celecoxib, Chlortalidone, Diclofenamide, Docetaxel, Nilotinib, Paclitaxel, Sunitinib, Vinorelbine, MDM2 Inhibitor, GANT-58, IWR-1-Endo, PHCCC, Valdecoxib, Givinostat, Pegpleranib, Dasatinib, Midostaurin, Nilotinib, Pazopanib, Sorafenib, Sunitinib, Imatinib, Vandetanib, Cabozantinib, Lenvatinib, DL-TBOA, Alda-1, Disulfiram, Prunetin, Butein, FK-888, GR-159897, PD-173074, PRV-3279, SM201, Valziflocept, WZ-7043, XmAb5871, 2-Aminopurine, Amiodarone, Baricitinib, Butein, Cinnarizine, Corticosterone, Cucurbitacin 1, Dalcetrapib, Dexamethasone, Dextromethorphan, Ellipticine, Eplerenone, Felodipine, Filgotinib, Flunarizine, Fluticasone, HG-5-113-01, Hydrocortisone, Mibefradil, Nitrendipine, Olaparib, Oxalomalic Acid, PG-9, Prednisolone, Progesterone, RS-102895, RS-504393, Sinensetin, Solcitinib, Tofacitinib, UB-165, Upadacitinib, Verapamil, ALW-II-38-3, ALW-II-49-7, Pepstatin, Tosedostat, 16,16-Dimethylprostaglandin E2, ALW-II-38-3, Axitinib, Cediranib, Cyclosporine, ENMD-2076, GTP-14564, Imatinib, Lenvatinib, Linifanib, Loperamide, MAZ-51, Motesanib, Nilotinib, Omalizumab, Pazopanib, PD-173074, Pexidartinib, Quizartinib, Semaxanib, Sorafenib, Sunitinib, Tivozanib, Verapamil, ZM-306416, 16,16-Dimethylprostaglandin E2, AMG-592, Aminogenistein, AZD1091, Cloprostenol, Fluprostenol, Iloprost, JW-7-24-1, Latanoprost, Low-dose IL-2, LY3471851, NSC-23766, PP-2, RG7835, Sirolimus, Travoprost, Butein, Chlortalidone, Hypericin, Indatraline, Isocarboxazid, Methoxsalen, Nialamide, Nomifensine, Omalizumab, Pargyline, Phenelzine, Piretanide, Tetrindole, Carfilzomib, Hypericin, Ixazomib, KZR-616, KZR-616 CONFIDENTIAL, L-803087, NU-7441, Oligomycin-C, Somatostatin, Zosuquidar, Aloisine, Alvocidib, Aminopurvalanol A, Amonafide, Amsacrine, AT-7519, BRD-K71726959, CGP-60474, Chlorpromazine, Daratumumab, Daunorubicin, Dinaciclib, Doxorubicin, Enrofloxacin, Etoposide, Hypericin, Idarubicin, Indirubin, Isatuximab, Ispinesib, JNJ-7706621, JW-67, Kenpaullone, Letrozole, Malonoben, Mitomycin C, Mitoxantrone, NSC-663284, NSC-693868, Ofloxacin, Olomoucine, PHA-793887, Pirarubicin, Purvalanol A, Purvalanol B, Razoxane, Riluzole, RO-3306, Roscovitine, TAK-079, Teniposide, or any combination thereof.

In some embodiments, the treatment for the disease or disorder comprises Anifrolumab, Deucravacitinib, Adalimumab, Certolizumab pegol, Etanercept, Golimumab, Infliximab, Palbociclib, Ribociclib, Abemaciclib, Anakinra, Canakinumab, Dasatinib, Apremilast, Roflumilast, or any combination thereof. In some embodiments, the treatment for the disease or disorder comprises Prednisone, Hydroxychloroquine, NSAIDS, Methotrexate (MTX), Cyclophosphamide (CTX), Mycophenolate mofetil (MMF), Azathioprine (AZA), Belimumab, Anifrolumab, Voclosporin, or any combination thereof. In some embodiments, the treatment for the disease or disorder comprises Anifrolumab, Deucravacitinib, Adalimumab, Certolizumab pegol, Etanercept, Golimumab, Inflximab, Palbociclib, Ribociclib, Abemaciclib, Anakinra, Canakinumab, Dasatinib, Apremilast, Roflumilast, Prednisone, Hydroxychloroquine, NSAIDS, Methotrexate (MTX), Cyclophosphamide (CTX), Mycophenolate mofetil (MMF), Azathioprine (AZA), Belimumab, Anifrolumab, Voclosporin, or any combination thereof.

In some embodiments, the treatment for the disease or disorder comprises Heliomycin, Enalapril, Perindopril, Phenelzine, Digitoxin, BI 655064, Bleselumab, Dapirolizumab Pegol, FFP104, Iscalimab, N-Acetyl Cysteine, VAY736, AM-281, AM-404, Amylocaine, Arachidonamide, Diclofenac, Dopamine, GW-405833, JBT-101, JTE-907, JWH-015, Lamotrigine, LY3361237, Mexiletine, Oxcarbazepine, Polatuzumab Vedotin, PRV-3279, Riluzole, Disulfiram, Dopamine, Fusaric Acid, ALW-II-38-3, Amoxapine, Chlorpromazine, Clobenpropit, Clozapine, Dilazep, Dorsomorphin, Immepip, Iodophenpropit, Loxapine, N6-Cyclopentyladenosine, Troglitazone, YM-90709, Belinostat, BMS-191011, Creatine, Diazoxide, ENMD-2076, NS-1619, Panobinostat, PT-2385, SID-7969543, Sulpiride, TC-S-7009, U-74389F, Vorinostat, 2,3-DCPE, Bisbenzimide, CP-466722, GK921, Gossypol, HA-14-1, KU-55933, LDN-27219, Navitoclax, TW-37, Fingolimod, Valproic Acid, CFM-1571, Linifanib, ODQ, Quizartinib, Semaxanib, Sorafenib, Sunitinib, Vandetanib, or any combination thereof.

In some embodiments, the treatment for the disease or disorder comprises Rituximab, Obinutuzmab, Ineilizumab, Ocrelizumab, Ofatumumab, Bortezomib, Carfilzomib, Ixazomib, Daratumumab, Isatuximab, Elotuzumab, or any combination thereof. In some embodiments, the treatment for the disease or disorder comprises duloxetine, gabapentin, milnacipran, pregabalin, or any combination thereof. In some embodiments, the treatment for the disease or disorder comprises Rituximab, Obinutuzmab, Ineilizumab, Ocrelizumab, Ofatumumab, Bortezomib, Carfilzomib, Ixazomib, Daratumumab, Isatuximab, Elotuzumab, duloxetine, gabapentin, milnacipran, pregabalin, or any combination thereof. In some embodiments, the disease or disorder comprises physical therapy, occupational therapy, psychological counseling, mindfulness and/or other forms of meditation training, alcohol intake reduction, sleep hygiene training, dietary changes including observance of a keto plan (reductions of carbohydrate intake), or any combination thereof.

In some embodiments, non-limiting examples of IFN inhibitors comprise anifrolumab, and deucravacitinib. In some embodiments, non-limiting examples of TNF inhibitor comprise adalimumab, certolizumab pegol, etanercept, golimumab, and infliximab. In some embodiments, non-limiting examples of cell cycle inhibitor comprise palbociclib, ribociclib, and abemaciclib. In some embodiments, non-limiting examples of IL-1 inhibitors comprise Anakinra and Canakinumab. In some embodiments, non-limiting examples of neutrophil function inhibitors comprise Dasatinib, Apremilast, and Roflumilast. In some embodiments, the treatment for the disease or disorder comprises a B cell inhibitor, a plasma cell inhibitor, an Ig chains inhibitor, neuromuscular pathways inhibitor, or any combination thereof. In some embodiments, non-limiting examples of B cell inhibitors comprise Rituximab, Obinutuzmab, Ineilizumab, Ocrelizumab, and Ofatumumab. In some embodiments, non-limiting examples of plasma cell inhibitors comprise Bortezomib, Carfilzomib, Ixazomib, Daratumumab, Isatuximab, and Elotuzumab.

In some embodiments, one or more genes selected from the genes listed in TABLE 2, TABLE 3, TABLE 4, or TABLE 5 is enriched in the biological sample, and the treatment comprises a drug as described herein. In some embodiments, one or more genes selected from the genes listed in TABLE 2, TABLE 3, TABLE 4, or TABLE 5 is enriched in the biological sample, and the treatment comprises a drug selected from: a immunoregulator, a immunosuppressant, a steroid, an anti-inflammatory, a JAK inhibitors, a TNF inhibitors, a baricitinib, a corticosteroid, a nonsteroidal anti-inflammatory drug (NSAID), a tofacitinib, a upadacitinib, a deucravacitinib, a brepocitinib, a disease-modifying anti-rheumatic drug (DMARD), a conventional synthetic DMARD (csDMARD), a targeted synthetic DMARD (tsDMARD), a biologic DMARD (bDMARD), a biologic treatment, a TYK2 inhibitor, a TYK2/JAK inhibitor, a combination inhibitor, a monoclonal antibody, an anti-TNF biologic, anti-IL-6 biologic, anti-IL-17 biologic, anti-IL-12/23 biologic, and anti-CD28 biologic, or combinations thereof. In some embodiments, one or more genes selected from the genes listed in TABLE 2, TABLE 3, TABLE 4, or TABLE 5 is enriched in the biological sample, and the treatment comprises an immunoregulator. In some embodiments, one or more genes selected from the genes listed in TABLE 2, TABLE 3, TABLE 4, or TABLE 5 is enriched in the biological sample, and the treatment comprises an immunosuppressant. In some embodiments, one or more genes selected from the genes listed in TABLE 2, TABLE 3, TABLE 4, or TABLE 5 is enriched in the biological sample, and the treatment comprises a steroid. In some embodiments, one or more genes selected from the genes listed in TABLE 2, TABLE 3, TABLE 4, or TABLE 5 is enriched in the biological sample, and the treatment comprises an anti-inflammatory. In some embodiments, one or more genes selected from the genes listed in TABLE 2, TABLE 3, TABLE 4, or TABLE 5 is enriched in the biological sample, and the treatment comprises a JAK inhibitor. In some embodiments, one or more genes selected from the genes listed in TABLE 2, TABLE 3, TABLE 4, or TABLE 5 is enriched in the biological sample, and the treatment comprises a TNF inhibitor. In some embodiments, one or more genes selected from the genes listed in TABLE 2, TABLE 3, TABLE 4, or TABLE 5 is enriched in the biological sample, and the treatment comprises a baricitinib. In some embodiments, one or more genes selected from the genes listed in TABLE 2, TABLE 3, TABLE 4, or TABLE 5 is enriched in the biological sample, and the treatment comprises a corticosteroid. In some embodiments, one or more genes selected from the genes listed in TABLE 2, TABLE 3, TABLE 4, or TABLE 5 is enriched in the biological sample, and the treatment comprises a nonsteroidal anti-inflammatory drug (NSAID). In some embodiments, one or more genes selected from the genes listed in TABLE 2, TABLE 3, TABLE 4, or TABLE 5 is enriched in the biological sample, and the treatment comprises a tofacitinib. In some embodiments, one or more genes selected from the genes listed in TABLE 2, TABLE 3, TABLE 4, or TABLE 5 is enriched in the biological sample, and the treatment comprises a upadacitinib. In some embodiments, one or more genes selected from the genes listed in TABLE 2, TABLE 3, TABLE 4, or TABLE 5 is enriched in the biological sample, and the treatment comprises a deucravacitinib. In some embodiments, one or more genes selected from the genes listed in TABLE 2, TABLE 3, TABLE 4, or TABLE 5 is enriched in the biological sample, and the treatment comprises a brepocitinib. In some embodiments, one or more genes selected from the genes listed in TABLE 2, TABLE 3, TABLE 4, or TABLE 5 is enriched in the biological sample, and the treatment comprises a disease-modifying anti-rheumatic drug (DMARD). In some embodiments, one or more genes selected from the genes listed in TABLE 2, TABLE 3, TABLE 4, or TABLE 5 is enriched in the biological sample, and the treatment comprises a conventional synthetic DMARD (csDMARD). In some embodiments, one or more genes selected from the genes listed in TABLE 2, TABLE 3, TABLE 4, or TABLE 5 is enriched in the biological sample, and the treatment comprises a targeted synthetic DMARD (tsDMARD). In some embodiments, one or more genes selected from the genes listed in TABLE 2, TABLE 3, TABLE 4, or TABLE 5 is enriched in the biological sample, and the treatment comprises a biologic DMARD (bDMARD). In some embodiments, one or more genes selected from the genes listed in TABLE 2, TABLE 3, TABLE 4, or TABLE 5 is enriched in the biological sample, and the treatment comprises a biologic treatment. In some embodiments, one or more genes selected from the genes listed in TABLE 2, TABLE 3, TABLE 4, or TABLE 5 is enriched in the biological sample, and the treatment comprises a TYK2 inhibitor. In some embodiments, one or more genes selected from the genes listed in TABLE 2, TABLE 3, TABLE 4, or TABLE 5 is enriched in the biological sample, and the treatment comprises a TYK2/JAK inhibitor. In some embodiments, one or more genes selected from the genes listed in TABLE 2, TABLE 3, TABLE 4, or TABLE 5 is enriched in the biological sample, and the treatment comprises a combination inhibitor. In some embodiments, one or more genes selected from the genes listed in TABLE 2, TABLE 3, TABLE 4, or TABLE 5 is enriched in the biological sample, and the treatment comprises a monoclonal antibody. In some embodiments, one or more genes selected from the genes listed in TABLE 2, TABLE 3, TABLE 4, or

TABLE 5 is enriched in the biological sample, and the treatment comprises an anti-TNF biologic. In some embodiments, one or more genes selected from the genes listed in TABLE 2, TABLE 3, TABLE 4, or TABLE 5 is enriched in the biological sample, and the treatment comprises anti-IL-6 biologic. In some embodiments, one or more genes selected from the genes listed in TABLE 2, TABLE 3, TABLE 4, or TABLE 5 is enriched in the biological sample, and the treatment comprises anti-IL-17 biologic. In some embodiments, one or more genes selected from the genes listed in TABLE 2, TABLE 3, TABLE 4, or TABLE 5 is enriched in the biological sample, and the treatment comprises anti-IL-12/23 biologic. In some embodiments, one or more genes selected from the genes listed in TABLE 2, TABLE 3, TABLE 4, or TABLE 5 is enriched in the biological sample, and the treatment comprises anti-CD28 biologic. In some embodiments, one or more genes selected from the genes listed in TABLE 2, TABLE 3, TABLE 4, or TABLE 5 is enriched in the biological sample, and the treatment comprises any one of the drugs described herein. In some embodiments, one or more genes selected from the genes listed in TABLE 2, TABLE 3, TABLE 4, or TABLE 5 is enriched in the biological sample, and the treatment comprises more than one of the drugs described herein.

In some embodiments, the methods described herein comprise accurate, repeatable, real-time, determinations of a disease or disorder in a patient (e.g., classifying a disease or disorder, determining progression of disease or disorder, treatment of a disease or disorder, predicting a clinical outcome of a disease or disorder), useful in developing targeted personalized treatment for patients with chronic, inflammatory, and/or autoimmune diseases as described herein. In some embodiments, methods of treatment described herein comprise at least two subsets of patients, each of the at least two subsets of patients corresponding to a different disease phenotype of an established disease or disorder. In some embodiments, at least two subsets of patients corresponding to a different disease phenotype respond to a different treatment. In some embodiments, at least two subsets of patients corresponding to a different disease phenotype correlate to a different treatment group.

In some embodiments, the methods described herein comprise determining progression of disease or disorder, predicting a clinical outcome of a disease or disorder, and, if necessary, modifying or adjusting the treatment of a disease or disorder to improve the clinical outcome of a disease or disorder. In some embodiments, adjusting the treatment of a disease or disorder comprises an adjustment of assay frequency and/or frequency of identification of disease or disorder. In some embodiments, adjusting the treatment of a disease or disorder comprises an adjustment of the method of treatment. In some embodiments, an adjustment of a method of treatment comprises administration of a different treatment, administration of a different drug(s), a different schedule of administration, or combinations thereof. In some embodiments, the administration comprises administration of more than one drug or treatment. In some embodiments, the administration of a drug or treatment is adjusted as a function of the progression of the disease or disorder. In some embodiments, the administration schedule is adjusted as a function of the progression of the disease or disorder or patient deterioration. In some embodiments, the progression of the disease or disorder takes into account more than one clinical indication. In some embodiments, the progression of the disease or disorder is related to overall patient deterioration.

In some embodiments, the method further comprises monitoring the disease or disorder of the patient, wherein the monitoring comprises assessing the disease or disorder of the patient at a plurality of different time points. A difference in the assessment of the disease or disorder of the patient among the plurality of time points can be indicative of one or more clinical indications selected from the group consisting of: (i) a classification of the disease or disorder of the patient, (ii) a prognosis of the disease or disorder of the patient, and (iii) an efficacy or non-efficacy of a course of treatment for treating the disease or disorder of the patient. In some embodiments, the patient has not been administered a treatment, and the method can predict an efficacy or non-efficacy of the treatment for treating the disease or disorder of the patient. In some embodiments, the patient has been administered a treatment, and the method can assess an efficacy or non-efficacy of the treatment for treating the disease or disorder of the patient. In some embodiments, the patient has been administered more than one treatment, and the method can assess an efficacy or non-efficacy of the more than one treatment for treating the disease or disorder of the patient.

### Administration of Drug

In some embodiments, the methods, systems, devices and compositions described herein are used for treating, preventing, or inhibiting a disease or disorder. In some embodiments, the methods described herein comprise methods of treatment. In some embodiments, the methods described herein comprise treatment of a disease or disorder in a patient. In some embodiments, the treatment of a disease or disorder comprises the administration of a drug described herein. In some embodiments, the treatment of a disease or disorder comprises the administration of a drug directed to the treatment targets of the disease or disorder. In some embodiments, the treatment of a disease or disorder comprises administration of a drug and/or a pharmaceutical composition. In some embodiments, the treatment of a disease or disorder comprises administration of one or more drugs and/or one or more pharmaceutical compositions. In some embodiments, the treatment of a disease or disorder comprises parenteral administration or oral administration of a drug. In some embodiments, the treatment of a disease or disorder comprises parenteral administration of a drug. In some embodiments, the treatment of a disease or disorder comprises oral administration of a drug.

In some embodiments, the treatment of a disease or disorder comprises parenteral administration of a drug, a medicament, a composition or a formulation. In some embodiments, the parenteral administration comprises administration to a patient or subject by a route other than topical or oral (i.e., a non-topical and non-oral route). Examples of parenteral routes include subcutaneous, intramuscular, intravascular (including intraarterial or intravenous), intraperitoneal, intraorbital, retrobulbar, peribulbar, intranasal, intrapulmonary, intrathecal, intraventricular, intraspinal, intracisternal, intracapsular, intrasternal or intralesional administration. Parenteral administration may be, e.g., by bolus injection or continuous infusion, either constant or intermittent and/or pulsatile, and may be via a needle or via a catheter or other tubing.

In some embodiments, the treatment comprises a drug, a medicament, a composition, a pharmaceutical composition, a formulation, or combinations thereof. In some embodiments, a drug, a medicament, a composition, a pharmaceutical composition, or a formulation is suitable for parenteral administration to a patient in need thereof. In some embodiments, the treatment is suitable for parenteral administration to a patient in need thereof. In some embodiments, the treatment is suitable for parenteral administration to a patient in need thereof and includes only pharmaceutically acceptable excipients, diluents, carriers and adjuvants that are safe for parenteral administration to humans at the concentrations used, under the same or similar standards as for excipients, diluents, carriers and adjuvants deemed safe by the Federal Drug Administration or other foreign national authorities. In some embodiments, treatment comprises a ready-to-use solution form, concentrated form, or a lyophilized preparation that may be reconstituted with a directed amount of diluent suitable for parenteral injection such as water, salt solution, or buffer solution.

In some embodiments, the treatment of a disease or disorder comprises oral administration of a drug, a medicament, a composition or a formulation. In some embodiments, the oral administration comprises administration to a patient or subject by a route other than topical or parenteral (i.e., a non-topical and non-parenteral route).

In some embodiments, the treatment comprises a drug, a medicament, a composition, a pharmaceutical composition, a formulation, or combinations thereof. In some embodiments, a drug, a medicament, a composition, a pharmaceutical composition, or a formulation is suitable for oral administration to a patient in need thereof. In some embodiments, the treatment is suitable for oral administration to a patient in need thereof. In some embodiments, the treatment is suitable for oral administration to a patient in need thereof and includes only pharmaceutically acceptable excipients, carriers, diluents, and/or adjuvants that are safe for oral administration to humans at the concentrations used, under the same or similar standards as for excipients, diluents, carriers and adjuvants deemed safe by the Federal Drug Administration or other foreign national authorities. In some embodiments, treatment comprises a ready-to-use solution form, concentrated form, or a lyophilized preparation that may be reconstituted with a directed amount of diluent suitable for oral injection such as water, salt solution, or buffer solution.

In some embodiments, the pharmaceutically acceptable excipient, carrier and/or diluent, is any substance formulated alongside the active ingredient of a pharmaceutical composition that allows the active ingredient to retain biological activity and is non-reactive with the patient's immune system. In some embodiments, the pharmaceutically acceptable excipient, carrier and/or diluent is comprised for the purpose of long-term stabilization, bulking up solid formulations that comprise potent active ingredients in small amounts, or to confer a therapeutic enhancement on the active ingredient in the final dosage form, such as facilitating absorption, reducing viscosity, or enhancing solubility. In some embodiments, the pharmaceutically acceptable excipient, carrier and/or diluent is selected as appropriate as related to the route of administration and the dosage form, as well as the active ingredient and other factors. In some embodiments, a composition comprising a pharmaceutically acceptable excipient, carrier and/or diluent is formulated by suitable methods as understood in the art.

### Disease or Disorder

The present disclosure comprises methods, systems, and kits are associated to the identification and/or treatment of a disease or disorder. In some embodiments, the methods, systems, and kits described herein comprise the identification, diagnosis, treatment, prevention, or inhibition of a disease or disorder of a patient. In some embodiments, the methods, systems, and kits described herein comprise predicting a clinical outcome of a disease or disorder of the patient.

In some embodiments, the methods, systems, and kits described herein are associated to the treatment of a disease or disorder in a patient. In some embodiments, the methods, systems, and kits described herein are associated to the treatment of more than one disease or disorder in a patient. In some embodiments, the disease or disorder is a chronic condition, an inflammatory condition, an autoimmune condition, an arthritis, a rheumatoid arthritis (RA), an early inflammatory arthritis (EIA), an inflammatory arthritis, a psoriatic arthritis (PSA), a lupus arthritis, a rhupus, an osteoarthritis, a non-inflammatory arthritis, a pauci-inflammatory arthritis, or combinations thereof. In some embodiments, the disease or disorder is a non-inflammatory arthritis such as osteoarthritis.

In some embodiments, the disease or disorder is Arthritis, Rheumatoid Arthritis (RA), Early Inflammatory Arthritis (EIA), Inflammatory Arthritis, Psoriatic Arthritis (PSA), Ankylosing spondylitis, Gout, Juvenile arthritis, Juvenile idiopathic arthritis, Osteoarthritis, Reactive arthritis, Septic arthritis, rhupus, rhupus syndrome, lupus arthritis, osteoarthritis, non-inflammatory arthritis, pauci-inflammatory arthritis, lupus, systemic lupus erythematosus (SLE), Type 1 SLE, Type 2 SLE, incomplete lupus erythematosus, lupus nephritis, Minimal mesangial lupus nephritis, Mesangial proliferative lupus nephritis, Focal lupus nephritis, Diffuse segmental nephritis, Membranous lupus nephritis, Advanced sclerosing lupus nephritis, acute cutaneous lupus erythematosus (ACLE), chronic cutaneous lupus erythematosus (CCLE), discoid lupus erythematosus (DLE), chilblain lupus erythematosus, subacute cutaneous lupus erythematosus (SCLE), cutaneous lupus, neonatal lupus, drug-induced lupus, drug-induced lupus erythematosus, or asymptomatic autoimmunity.

### Kits

In some embodiments, compositions and/or system components are assembled in a kit. Accordingly, disclosed herein are kits for diagnosis, treatment, prevention, or inhibition of a disease or disorder in a patient. In some embodiments, kits are compatible with methods for identifying a disease or disorder. In some embodiments, kits are compatible with methods for diagnosis of a disease or disorder. In some embodiments, kits are compatible with methods for identifying the effectiveness of a treatment of a disease or disorder. In some embodiments, kits are compatible with methods for predicting a clinical outcome of a disease or disorder described herein. In some embodiments, kits are compatible with methods for prevention of a disease or disorder. In some embodiments, kits are compatible with methods for treatment of a disease or disorder. In some embodiments, kits are compatible with methods of identifying a disease or disorder of a patient or a susceptibility of a patient. In some embodiments, kits are compatible with methods of identifying a treatment of disease or disorder. In some embodiments, kits are compatible with methods of identifying the effectiveness of a treatment of disease or disorder. In some embodiments, the effectiveness of a treatment is measured as a function of the progression of the disease or disorder. In some embodiments, the kits described herein are associated to a disease or disorder as described herein. In some embodiments, the kits described herein are associated to a treatment of a disease or disorder as described herein. In some embodiments, kits are compatible with methods disclosed herein, including methods for predicting a clinical outcome of a disease or disorder of a patient.

Any of the kits described herein are compatible with any of the compositions, systems, kits, or methods disclosed herein. In some embodiments, the kits described herein are compatible with methods related to identifying a disease or disorder, identifying a susceptibility to a disease or disorder, diagnosis of a disease or disorder, predicting a clinical outcome of a disease or disorder of a patient, or combinations thereof. By way of non-limiting example, in some embodiments, the kits described herein are used in identifying a disease or disorder. In some embodiments, the kits described herein are used in identifying a susceptibility to a disease or disorder. In some embodiments, the kits described herein are used in diagnosis of a disease or disorder. By way of non-limiting example, in some embodiments, the kits described herein are used in predicting a clinical outcome of a disease or disorder of a patient.

In some embodiments, kits are compatible with methods as disclosed herein, wherein a kit further comprises a detectable label or a nucleic acid encoding a detectable label. In some embodiments, the components of the kit are in same compriseer. In some embodiments, the components of the kit are in separate compriseers.

In some embodiments, a kit for performing a method as described herein, the kit comprising a structural component as well as a composition component, wherein the composition component is a reaction mixture comprising an isolated biological sample from a patient and composition components and/or reagents for performing the assaying. In some embodiments, the structural component comprises a sample interface. In some embodiments, the structural component comprises a computer comprising a computer-readable storage media or a non-transitory computer-readable storage media encoded with a computer program including instructions executable by a processor to run an application for identifying and comparing (i) the gene expression data generated from assaying the isolated biological sample to (ii) a reference gene expression data set from one or more gene modules.

### Kit Components

In general, kit components comprise structural components as well as sample components, including compositions and systems described herein. Often, kits comprise one or more containers compatible for containing the samples, compositions, and systems described herein. In some embodiments, components of the samples, compositions, and systems are contained in the same container or in separate containers. In some embodiments, a container is a syringe, test wells, bottles, chambers, channels, vials, or test tubes. In one embodiment, the containers are formed from a variety of materials such as glass, plastic, or polymers. The system or systems described herein comprise packaging materials. Examples of packaging materials comprise, but are not limited to, pouches, blister packs, bottles, tubes, bags, containers, bottles, and any packaging material suitable for intended mode of use.

In some embodiments, a kit comprises components, compositions, systems, and/or reagents for performing any methods disclosed herein. In some embodiments, a kit comprises components, compositions, and/or reagents for performing an assay disclosed herein. In some embodiments, a kit comprises other therapeutic agents, carriers, buffers, containers, and/or devices for administration. In some embodiments, kits described herein comprise a solid support. In some embodiments, a nucleic acid as described herein is attached to a solid support. For example, in some embodiments, the solid support is a gel, an electrode, or a bead. In some embodiments, the solid support is a gel. In some embodiments, the bead is a magnetic bead. In some embodiments, the nucleic acid flows through a chamber into a mixture comprising a substrate. When the nucleic acid meets the substrate, a reaction occurs (e.g., a colorimetric reaction), which is then detected.

In some embodiments, the kit comprises labels and/or instructions for performing a method as described herein. In some embodiments, the kit comprises labels and/or instructions for use. In some embodiments, labeling and/or instructions comprise, for example, information concerning the amount, frequency and method of introduction and/or administration of the compositions, systems, and/or nucleic acid constructs described herein. In some embodiments, a label is on a container when letters, numbers or other characters forming the label are attached, molded, or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein. After packaging the formed product and wrapping or boxing to maintain a sterile barrier, in some embodiments, the product is terminally sterilized by heat sterilization, gas sterilization, gamma irradiation, or by electron beam sterilization. Alternatively, in some embodiments, the product is prepared and packaged by aseptic processing.

In some embodiments, the instructions for practicing the methods are recorded on a suitable recording medium. In some embodiments, the instructions are printed on a substrate, such as paper or plastic, etc. In some embodiments, the instructions are present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g., CD-ROM, diskette, flash drive, etc. In some embodiments, the actual instructions are not present in the kit, but means for obtaining the instructions from a remote source (e.g. via the Internet), are provided. In some embodiments, the kit comprises a web address where the instructions are viewed and/or from which the instructions are downloaded.

### Tables

TABLE 6 provides illustrative sample traits for systems and methods described herein.

TABLE 6
Sample Traits

high sensitivity C-reactive protein (CRP) level, blood C-reactive protein level, blood protein level,
blood complement component 3 (C3) protein level, blood complement component 4 (C4) protein
level, rheumatoid factor (RF) level, anti-CCP (ACPA) level, matrix metalloproteinase (MMP)-1
level, MMP-3 level, drug level, glucose level, cholesterol level, inflammatory marker level,
autoimmune marker level, antibody level, blood autoimmune antibody level, blood pressure,
erythrocyte sedimentation rate (ESR), disease activity score (DAS) score, disease activity score for
28 joints (DAS28 score), age, sex, ancestry, drug usage, disease duration, swollen joints, tender
joints, tender joint count (TJC), polysymptomatic distress scale (PSD), fibromyalgia score, total
areas of pain, patient gender (male being pt.is.female being 0 or white, female being pt.is.female
being 1 or colored), patient ancestral background (positive values of yes or colored indicating
African ancestry AA, Asian ancestry AsA, Caribbean ancestry Carib, European ancestry EA),
patient treatment arm (CZP, ETC), patient treatment responder level (none non, mod moderate, high
high), patient pauci-immune designation pt.histo.pauci.immune, patient biopsy location (wrist,
knee), patient indication of RA erosion, pathology designation (erosion, myeloid, lymphoid,
fibroid), patient age, patient inflammatory score (pt.score.inf), patient DAS28 score
(pt.score.das28), patient change in DAS28 score after treatment (pt.score.das28.delta), patient HAQ
score disease index (pt.score.haq.di), patient disease duration (pt.disease.duration), number of
swollen joints (pt.count.joint.swollen), number of tender joints (pt.count.joint.tender), CRP C-
reactive protein level (pt.CRP), patient erythrocyte sedimentation rate ESR (pt.ESR), patient
rheumatoid factor (pt.RF), patient ACPA level (pt.ACPA), patient blood sample RNA concentration
(pt.RNA.concentration), patient blood sample total RNA volume (pt.RNA.volume), patient blood
sample total RNA yield (pt.RNA.yield), PSD score (PSD.score), immunosuppressive drug usage,
duloxetine usage, amitriptyline usage, prednisone usage, SLEDAI, SLEDAI score, blood
autoimmune antibody level (anti.dsDNA), blood complement component 3 (C3) protein level
(complement_C3), blood C3 protein level, Age, whether of African ancestry (AA) (ancestry_AA,
yes = 1, no = 0), whether of European ancestry (EA) (ancestry_EA, yes = 1, no = 0), whether of
hispanic ancestry (HA) (ancestry_HA, yes = 1, no = 0), whether a female (is_female yes = 1, no = 0),
lu vas1 init (lu_vas1_init), lu vas2 init (lu_vas2_init), sledai arthritis (sledai_arthritis), sledai rash
(sledai_rash), sledai ulcers (sledai_ulcers), sledai pleurisy (sledai_pleurisy), sledai leukopenia
(sledai_leukopenia), sledai hematuria (sledai_hematuria), sledai pyuria (sledai_pyuria), sledai
proteinuria (sledai_proteinuria), fatigue severity (fatigue_severity), cognitive, wake unrefresh
(wake_unrefresh), headache symptom (headache_symptom), has cramps, has depression, total areas
of pain (total_areas_of_pain), total symptom severity (Total_Symptom_Severity), totalpain, month
flare (month_flare), week muscle (muscle_weak), muscle pain (muscle_pain), swell joints
(swell_joints), pain stiff joint (pain_stiff_joint), rash malar (rash_malar), rash sun (rash_sun),
vasculitis, rash_oth, weight loss (wt_loss), fatigue, fever, swollen glands (swollen_glands),
alopecia, dry eye mouth (dry_eye_mouth), sores_mouth_nose (sores_mouth_nose), Raynaud, short
breath (short_breath), pain deep breath (pain_deep_breath), forget, feel depressed (feel_depressed),
anxiety, headache, stroke, numb tingle (numb_tingle), belly pain (belly_pain), edema, hypertension,
upc, urine foamy (urine_foamy), urine pain (urine_pain), rate lupus (rate_lupus), type 1 lupus, type
2 lupus, er visit (er_visit), little interest (little_interest), depressed, sleep trouble (sleep_trouble),
tired, poor appetite (poor_appeti), disappoint, concentrate, slow fidget (slow_fidget), understand,
follow direct (follow_direct), miss dose (miss_dose), when missed (when_missed), percent med
(percent_med), hydroxychloroquine (HCQ) drug usage (drug.HCQ), HCQ drug usage, Prednisone
drug usage (drug.Prednisone), Cytoxan drug usage (drug.Cytoxan), Cellcept drug usage
(drug.CellCept), CellCept usage, mycophenolate mofetil (MMF) drug usage (drug.MMF),
Azathioprine drug usage (drug. Azathioprine), Methotrexate drug usage (drug.Methotrexate),
Benlysta drug usage (drug.Benlysta), Benlysta usage, Adalimumab drug usage (drug.Adalimumab),
Non-steroidal anti-inflammatory drugs usage, NSAIDs usage (drug.NSAIDs), Leflunomide drug
usage (drug.Leflunomide), ACE drug usage (drug.ACE), ARB drug usage (drug.ARB), Aspirin
drug usage (drug.Aspirin), Amlodipine drug usage (drug.Amlodipine), HCTZ drug usage
(drug.HCTZ), Lasix drug usage (drug.Lasix), Metoprolol drug usage (drug.Metoprolol), Coreg
drug usage (drug.Coreg), Chlorthalidone drug usage (drug.Chlorthalidone), Gabapentin drug usage
(drug.Gabapentin), Lyrica drug usage (drug.Lyrica), Flexeril drug usage (drug.Flexeril), Elavil drug
usage (drug.Elavil), Cymbalta drug usage (drug.Cymbalta), Cymbalta usage, Effexor drug usage
(drug.Effexor), Milnacipran drug usage (drug.Milnacipran), Wellbutrin drug usage
(drug.Wellbutrin), Tramadol drug usage (drug.Tramadol), or combinations thereof.

TABLE 7 provides illustrative gene function signature lists for systems and methods described herein.

TABLE 7
Gene Function Signature Lists

AMPEL LuGENE, AMPEL Ancestry (Anc), AMPEL Endotype.32 (Endo.32), Endotype.kidney,
AMPEL tissues (Tis), Biologically Informed Gene Clustering (BIG-C) signature, Gene Ontology
(GO) database, Hallmark gene sets, KEGG Pathway Database, Reactome signature, BRETIGEA
signature, Immune/Inflammation-Scope (I-Scope), Tissue-Scope (T-Scope), or combinations
thereof.

### EXAMPLES

The following illustrative examples are representative of embodiments of the software applications, systems, and methods described herein and are not meant to be limiting in any way.

### Example 1: Unsupervised Grouping of Genes into Gene Modules to Identify Subgroups of Inflammatory Arthritis Patients

Gene expression in fine-needle synovial biopsy samples from patients with early inflammatory arthritis was analyzed. Microarray data from the synovium of patients with early inflammatory arthritis underwent background correction and GCRMA normalization resulting in log 2 intensity values compiled into an expression set object (e-set). Three samples were removed for lack of patient metadata. As the ILLUM-1 analysis probes not mapping to a known HGNC protein were discarded. Averaged gene expression rows were sorted by absolute value of descending row variance and the top 5,000 row variance (inflammatory arthritis-top5k rowVar) genes among the remaining 17 early inflammatory arthritis samples were selected for further analysis. CodeR-BP technique was used to elucidate gene module enrichment patterns, regulatory networks, differentially expressed gene pairs within and between modules unique to each ancestral background, and identify specific subsets of patients, relationships with specific clinical or laboratory traits, from the study. The inflammatory arthritis top5k rowVar genes were grouped based on Gene Coexpression Network (GCN) generation and multi-scale module formation. Planar filtered network (PFN) generated requiring a correlation false discovery rate (FDR)<0.2, and ensuing multi-scale gene modules were generated using the public R MEGENA package. Minimum module size was 20 genes. A formal tree object was created to establish module lineage and assign module lineage names. Gene modules were assigned “lineage” names based on their multi-scale dependency from the root module. Module eigengenes (MEs) were calculated as the first principle component of the gene expression values within each module. Modules with MEs significantly correlated with MMP2 cohort (more inflammatory arthritis) as well as with serologic numerical assay measurements of inflammation including erythrocyte sedimentation rate (ESR), blood C-Reactive Protein level (CRP) were identified. The significant gene modules which were subsequently overlapped with various gene function signature lists selected from Immune/Inflammation-Scope (I-Scope), Tissue-Scope (T-Scope) and Biologically Informed Gene Clustering (BIG-C). I-Scope, T-Scope and BIG-C are functional aggregation tools for characterizing immune cells by type, tissue cells by type, and biologically classifying large groupings of genes, respectively. I-Scope categorizes gene transcripts into a possible 28 hematopoietic cell categories based on matching transcripts known to mark various types of immune/inflammatory cells. T-Scope is an additional aggregation tool to characterize cell types found in specific tissues. BIG-C sorts genes into 53 different groups based on their most probable biological function and/or cellular or subcellular localization. These transcriptomic signatures, along with others derived from literature [Catalina et al. 2020, Owen et al. 2020, Kingsmore et al. 2021, Daamen et al. 2021, & Culemann et al. 2019] and gene ontology (GO) terms, were utilized to calculate enrichment statistics among gene coexpression modules. Odds ratios and overlap p-values were calculated using Fisher's Exact test in R using the fisher.test( ) function. Statistical significance was obtained using an adjusted p-value≤0.2. Coexpression modules were annotated according to the top overlapping functional category with the most significant p-value and a minimum of 4 overlapping genes. In the absence of significant overlaps, “unknown” was the assigned annotation.

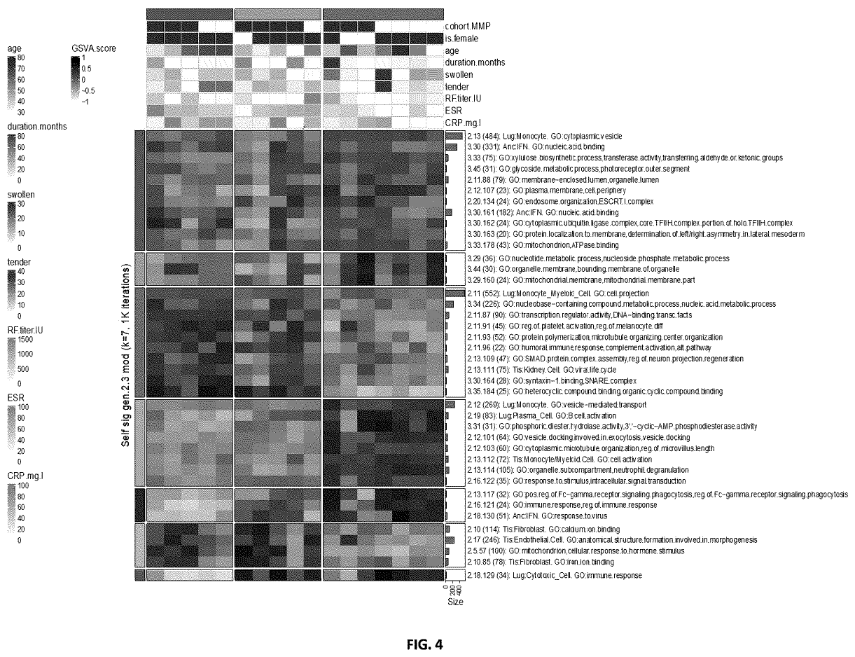

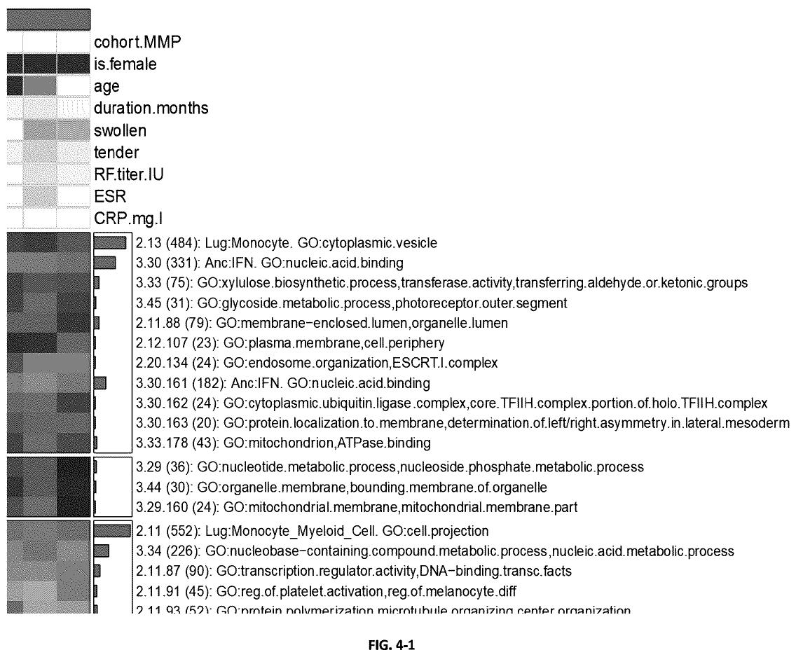

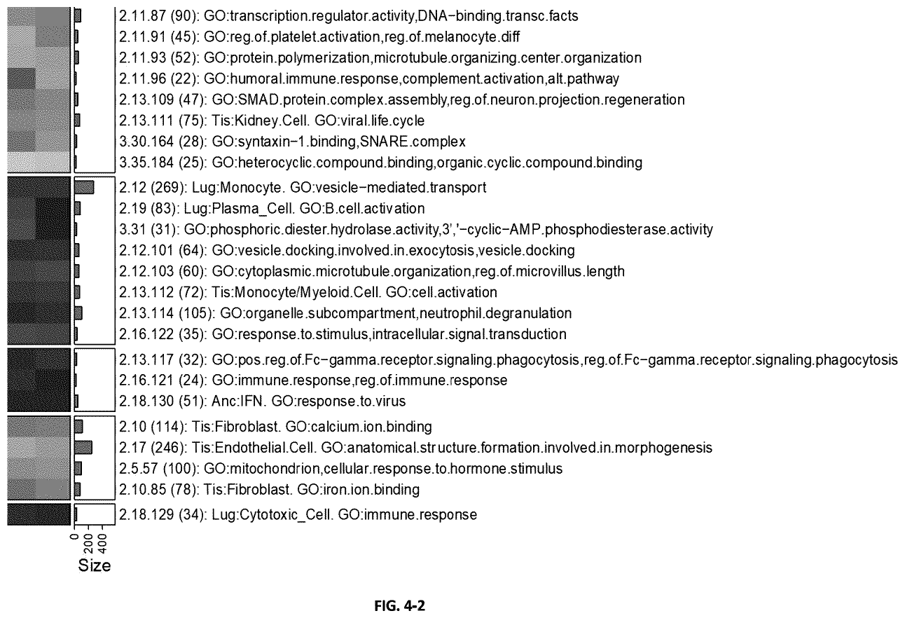

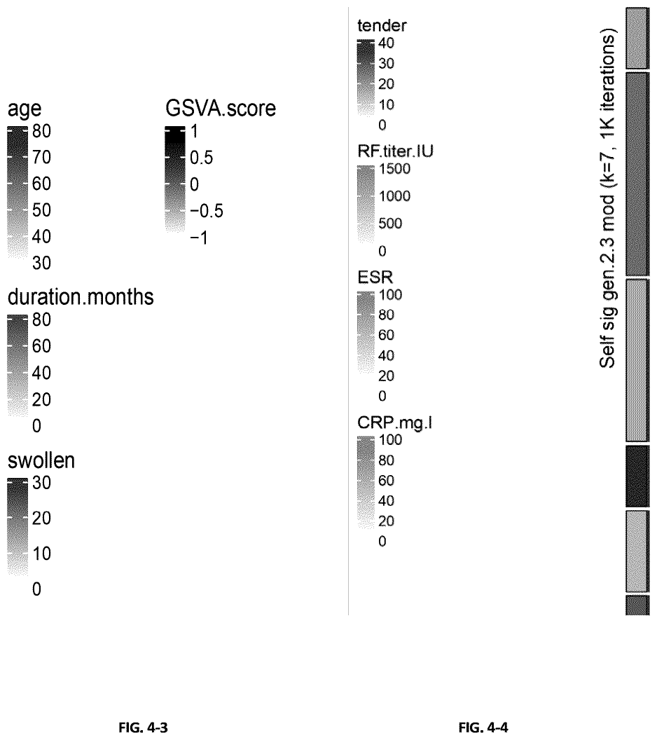

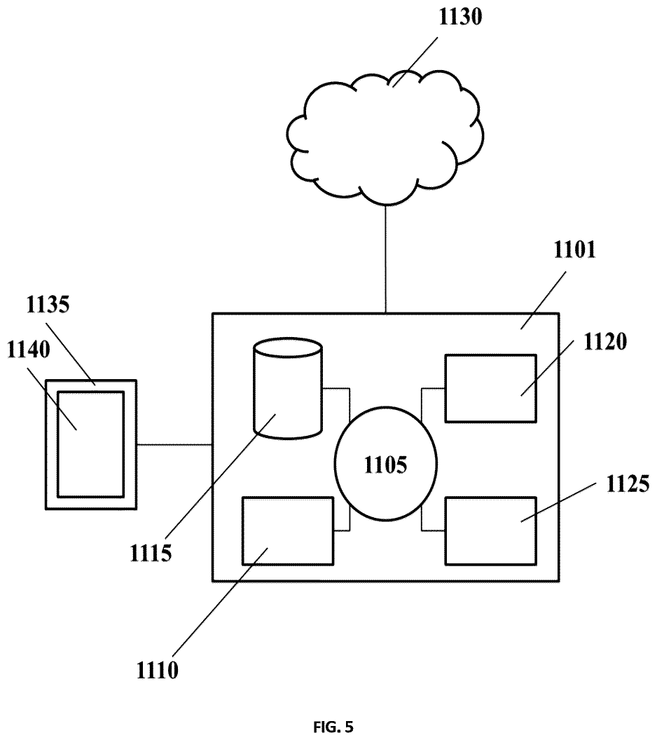

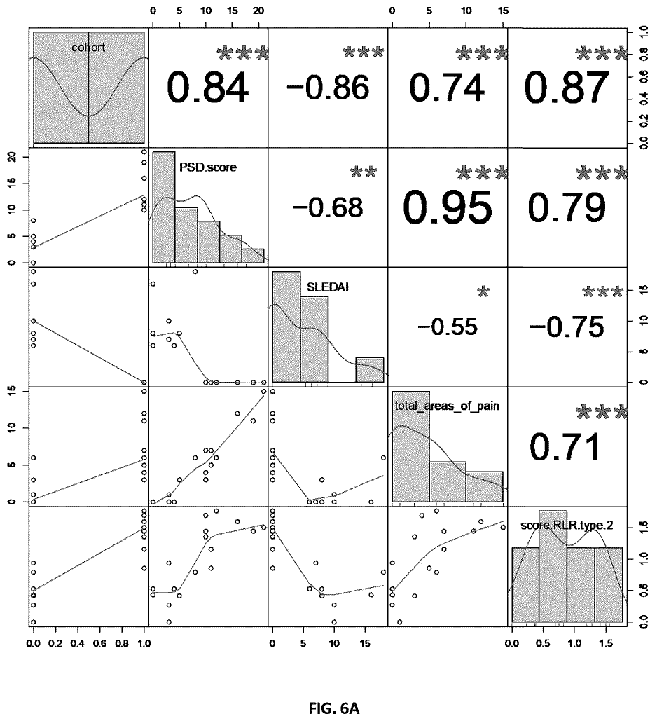

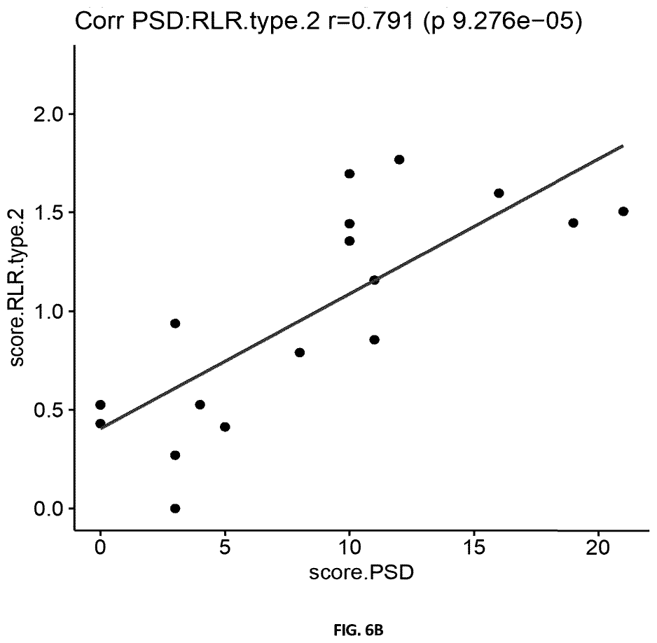

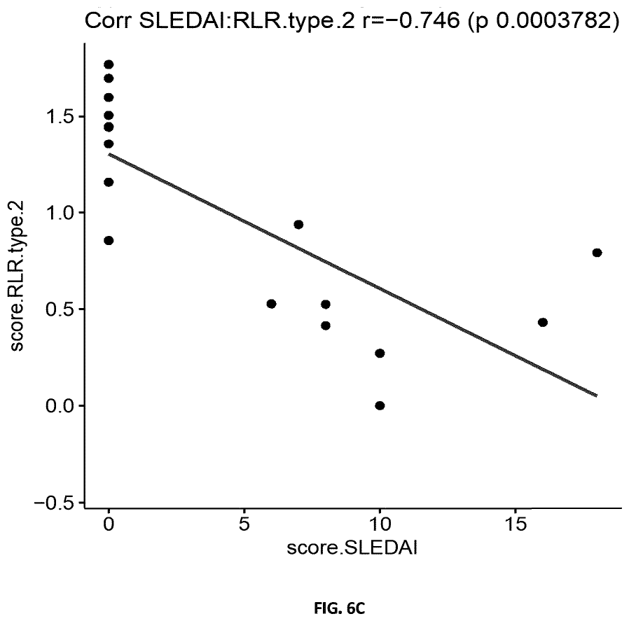

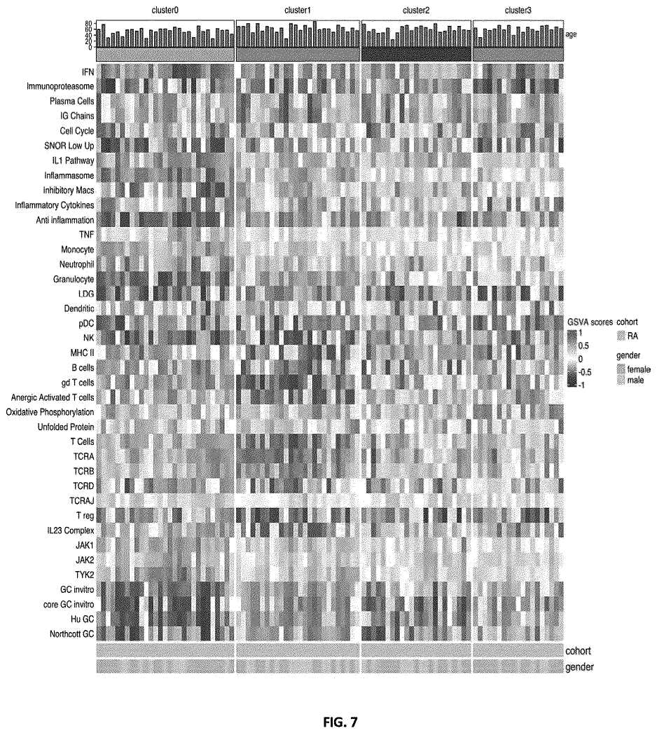

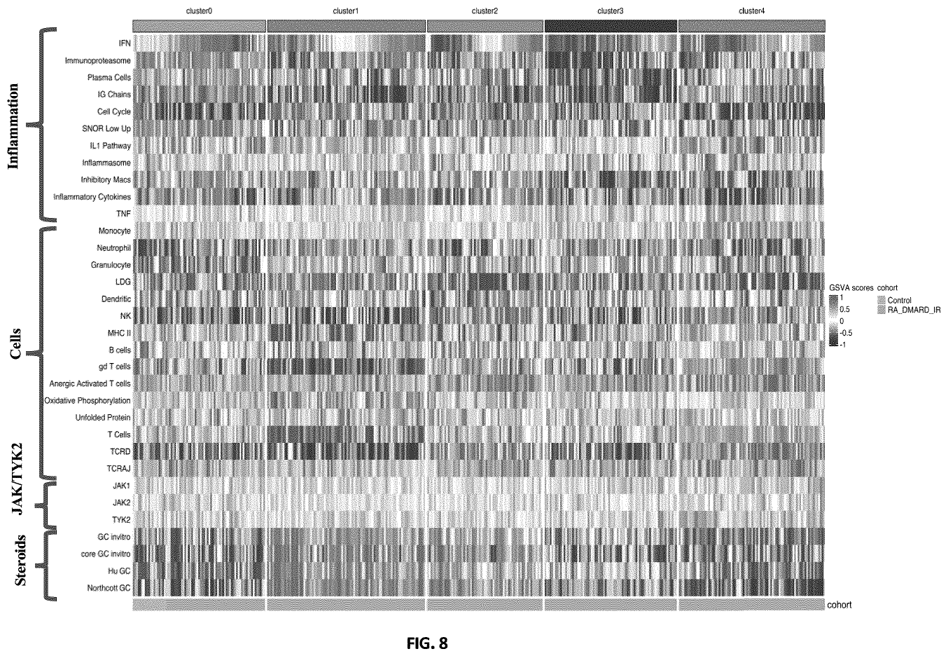

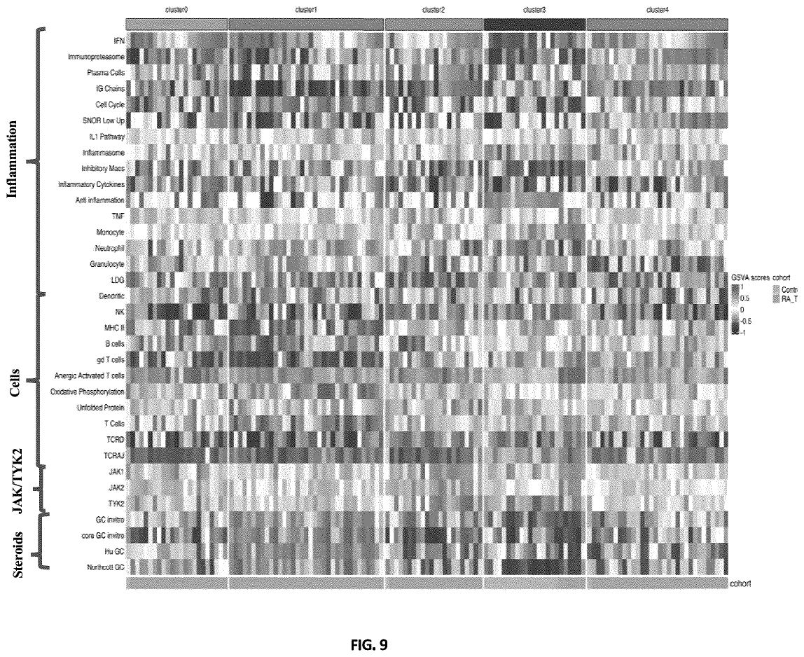

FIG. 4 shows correlation of the significant gene modules with cohort (more inflammatory arthritis) as well as with serologic features of inflammation including ESR and CRP. Numbers in each box indicate significant correlation coefficients (p.val<0.2). Column labels indicate clinical features. Rows were labeled by gene module names indicating lineage name, module size (number of genes, in parenthesis), followed by top significant cell type or functional annotations (e.g., functional characterization) selected from LuGENE, AMPEL Ancestry, AMPEL Tissue, BIG-C and GO (signature overlap >=4 gene symbols, Fisher's p.val<0.2). Table 1A, lists the significant gene modules (e.g., significantly correlated modules to anti.dsDNA) shown in FIG. 4, size (e.g., number of genes within the modules) of the modules, functional characterization groups (e.g., categories) from LuGENE, AMPEL Ancestry, AMPEL Tissue, BIG-C and GO that overlaps with the modules and respective p values, and functional annotation of the modules. Table 1B lists the genes in the significant gene modules of Table 1A.

TABLE 1A
The significant gene modules as shown in FIG. 4.

| | | | annot. | | annot. | | |
|---|---|---|---|---|---|---|---|
| | | | luGENE. | annot. | ancestry. | annot. | annot.tissue. |
| lineage | mod.size | annot.luGENE | overlaps | ancestry | overlaps | tissue | overlaps |
| 2.10 | 114 | | | | | Fibroblast | 8 |
| 2.10.85 | 78 | | | | | Fibroblast | 5 |
| 2.11 | 552 | Monocyte_ | 9 | | | | |
| | | Myeloid_Cell | | | | | |
| 2.11.87 | 90 | | | | | | |
| 2.11.88 | 79 | | | | | | |
| 2.11.91 | 45 | | | | | | |
| 2.11.93 | 52 | | | | | | |
| 2.11.96 | 22 | | | | | | |
| 2.12 | 269 | Monocyte | 4 | | | | |
| 2.12.101 | 64 | | | | | | |
| 2.12.103 | 60 | | | | | | |
| 2.12.107 | 23 | | | | | | |
| 2.13 | 484 | Monocyte | 5 | | | | |
| 2.13.109 | 47 | | | | | | |
| 2.13.111 | 75 | | | | | Kidney | 4 |
| | | | | | | Cell | |
| 2.13.112 | 72 | | | | | Monocyte/ | 5 |
| | | | | | | Myeloid | |
| | | | | | | Cell | |
| 2.13.114 | 105 | | | | | | |
| 2.13.117 | 32 | | | | | | |
| 2.16.121 | 24 | | | | | | |
| 2.16.122 | 35 | | | | | | |
| 2.17 | 246 | | | | | Endothelial | 6 |
| | | | | | | Cell | |
| 2.18.129 | 34 | Cytotoxic_ | 8 | | | | |
| | | Cell | | | | | |
| 2.18.130 | 51 | | | IFN | 19 | | |
| 2.19 | 83 | Plasma_Cell | 6 | | | | |
| 2.20.134 | 24 | | | | | | |
| 2.5.57 | 100 | | | | | | |
| 3.29 | 36 | | | | | | |
| 3.29.160 | 24 | | | | | | |
| 3.30 | 331 | | | IFN | 5 | | |
| 3.30.161 | 182 | | | IFN | 4 | | |
| 3.30.162 | 24 | | | | | | |
| 3.30.163 | 20 | | | | | | |
| 3.30.164 | 28 | | | | | | |
| 3.31 | 31 | | | | | | |
| 3.33 | 75 | | | | | | |
| 3.33.178 | 43 | | | | | | |
| 3.34 | 226 | | | | | | |
| 3.35.184 | 25 | | | | | | |
| 3.44 | 30 | | | | | | |
| 3.45 | 31 | | | | | | |
| | | annot.BIG. | | annot. | | | |
| | annot. | C.over | | GO.1. | annot. | annot. GO. | |
| Lineage | BIG.C | laps | annot.GO.1 | coverage | GO.2 | 2.coverage | annot.figures |
| 2.10 | | | calcium ion | 0.123 | positive | 0.026 | Tis:Fibroblast. |
| | | | binding | | regulation of | | GO:calcium.ion.binding |
| | | | | | megakaryocyte | | |
| | | | | | differentiation | | |
| 2.10.85 | | | iron ion | 0.064 | endothelial cell | 0.051 | Tis:Fibroblast. |
| | | | binding | | development | | GO:iron.ion.binding |
| 2.11 | | | cell | 0.174 | cytoplasmic | 0.054 | Lug:Monocyte_Myeloid_Cell. |
| | | | projection | | region | | GO:cell.projection |
| 2.11.87 | Cytoskeleton | 12 | transcription | 0.222 | DNA-binding | 0.189 | GO:transcription.regulator. |
| | | | regulator | | transcription | | activity, DNA- |
| | | | activity | | factor activity | | binding.transc.facts |
| 2.11.88 | mRNA- | 5 | membrane- | 0.506 | organelle | 0.506 | GO:membrane- |
| | Processing | | enclosed | | lumen | | enclosed.lumen,organelle. |
| | | | lumen | | | | lumen |
| 2.11.91 | Transcription- | 7 | regulation of | 0.067 | regulation of | 0.044 | GO:reg.of.platelet.activation, |
| | Factors | | platelet | | melanocyte | | reg.of.melanocyte.diff |
| | | | activation | | differentiation | | |
| 2.11.93 | Transporters | 4 | protein | 0.115 | microtubule | 0.077 | GO:protein.polymerization, |
| | | | polymeri- | | organizing | | microtubule.organizing. |
| | | | zation | | center | | center.organization |
| | | | | | organization | | |
| 2.11.96 | | | humoral | 0.136 | complement | 0.091 | GO:humoral.immune.response, |
| | | | immune | | activation, | | complement.activation, |
| | | | response | | alternative | | alt pathway |
| | | | | | pathway | | |
| 2.12 | | | vesicle- | 0.23 | leukocyte | 0.119 | Lug:Monocyte. |
| | | | mediated | | activation | | GO:vesicle-mediated.transport |
| | | | transport | | involved in | | |
| | | | | | immune | | |
| | | | | | response | | |
| 2.12.101 | Cytoskeleton | 7 | vesicle | 0.047 | vesicle docking | 0.047 | GO:vesicle.docking.involved. |
| | | | docking | | | | in.exocytosis, |
| | | | involved in | | | | vesicle.docking |
| | | | exocytosis | | | | |
| 2.12.103 | Mitochondria- | 4 | cytoplasmic | 0.05 | regulation of | 0.033 | GO:cytoplasmic.microtubule. |
| | General | | microtubule | | microvillus | | organization, reg.of. |
| | | | organization | | length | | microvillus.length |
| 2.12.107 | Immune-Cell- | 4 | plasma | 0.739 | cell periphery | 0.739 | GO:plasma.membrane, |
| | Surface | | membrane | | | | cell.periphery |
| 2.13 | | | cytoplasmic | 0.231 | leukocyte | 0.118 | Lug:Monocyte. |
| | | | vesicle | | mediated | | GO:cytoplasmic.vesicle |
| | | | | | immunity | | |
| 2.13.109 | | | SMAD | 0.043 | regulation of | 0.043 | GO:SMAD.protein.complex. |
| | | | protein | | neuron | | assembly, reg.of.neuron. |
| | | | complex | | projection | | projection.regeneration |
| | | | assembly | | regeneration | | |
| 2.13.111 | | | viral life | 0.093 | establishment | 0.08 | Tis:Kidney.Cell. |
| | | | cycle | | of vesicle | | GO:viral.life.cycle |
| | | | | | localization | | |
| 2.13.112 | | | cell | 0.319 | leukocyte | 0.25 | Tis:Monocyte/Myeloid.Cell. |
| | | | activation | | mediated | | GO:cell.activation |
| | | | | | immunity | | |
| 2.13.114 | Lysosome | 7 | organelle | 0.238 | neutrophil | 0.143 | GO:organelle.subcompartment, |
| | | | subcompart- | | degranulation | | neutrophil.degranulation |
| | | | ment | | | | |
| 2.13.117 | | | positive | 0.062 | regulation of | 0.062 | GO:pos.reg.of.Fc- |
| | | | regulation of | | Fc-gamma | | gamma.receptor.signaling. |
| | | | Fc-gamma | | receptor | | phagocytosis, reg.of. |
| | | | receptor | | signaling | | Fc-gamma.receptor. |
| | | | signaling | | pathway | | signaling.phagocytosis |
| | | | pathway | | involved in | | |
| | | | involved in | | phagocytosis | | |
| | | | phagocytosis | | | | |
| 2.16.121 | Immune-Cell- | 6 | immune | 0.542 | regulation of | 0.375 | GO:immune.response,reg.of. |
| | Surface | | response | | immune | | immune.response |
| | | | | | response | | |
| 2.16.122 | General-Cell- | 7 | response to | 0.829 | intracellular | 0.486 | GO:response.to.stimulus, |
| | Surface | | stimulus | | signal | | intracellular.signal. |
| | | | | | transduction | | transduction |
| 2.17 | | | anatomical | 0.22 | vasculature | 0.187 | Tis:Endothelial.Cell. |
| | | | structure | | development | | GO:anatomical.structure. |
| | | | formation | | | | formation.involved.in. |
| | | | involved in | | | | morphogenesis |
| | | | morpho- | | | | |
| | | | genesis | | | | |
| 2.18.129 | | | immune | 0.588 | lymphocyte | 0.382 | Lug:Cytotoxic_Cell. |
| | | | response | | activation | | GO:immune.response |
| 2.18.130 | | | response to | 0.412 | defense | 0.392 | Anc:IFN. |
| | | | virus | | response to | | GO:response.to.virus |
| | | | | | virus | | |
| 2.19 | | | B cell | 0.108 | adaptive | 0.108 | Lug:Plasma_Cell. |
| | | | activation | | immune | | GO:B.cell.activation |
| | | | | | response | | |
| 2.20.134 | | | endosome | 0.083 | ESCRT I | 0.042 | GO:endosome.organization, |
| | | | organization | | complex | | ESCRT.I.complex |
| 2.5.57 | Mitochondria | 10 | mitochondrion | 0.23 | cellular | 0.16 | GO:mitochondrion, |
| | -General | | | | response to | | cellular.response.to.hormone. |
| | | | | | hormone | | stimulus |
| | | | | | stimulus | | |
| 3.29 | Secreted-and- | 4 | nucleotide | 0.222 | nucleoside | 0.222 | GO:nucleotide.metabolic. |
| | ECM | | metabolic | | phosphate | | process,nucleoside.phosphate. |
| | | | process | | metabolic | | metabolic.process |
| | | | | | process | | |
| 3.29.160 | | | mitochondrial | 0.292 | mitochondrial | 0.208 | GO:mitochondrial.membrane, |
| | | | membrane | | membrane part | | mitochondrial.membrane. |
| | | | | | | | part |
| 3.30 | | | nucleic acid | 0.266 | RNA | 0.082 | Anc:IFN. |
| | | | binding | | processing | | GO:nucleic.acid. binding |
| 3.30.161 | | | nucleic acid | 0.308 | catalytic | 0.143 | Anc IFN. |
| | | | binding | | complex | | GO:nucleic.acid.binding |
| 3.30.162 | | | cytoplasmic | 0.042 | core TFIIH | 0.042 | GO:cytoplasmic.ubiquitin. |
| | | | ubiquitin | | complex | | ligase.complex, |
| | | | ligase | | portion of holo | | core.TFIIH.complex.portion. |
| | | | complex | | TFIIH complex | | of.holo.TFIIH.complex |
| 3.30.163 | Unknown | 4 | protein | 0.2 | determination | 0.05 | GO:protein.localization.to. |
| | | | localization | | of left/right | | membrane,determination.of. |
| | | | to membrane | | asymmetry in | | left/right.asymmetry.in.lateral. |
| | | | | | lateral | | mesoderm |
| | | | | | mesoderm | | |
| 3.30.164 | Transcription- | 4 | syntaxin-1 | 0.071 | SNARE | 0.071 | GO:syntaxin-1.binding, |
| | Factors | | binding | | complex | | SNARE.complex |
| 3.31 | Unknown | 4 | phosphoric- | 0.097 | 3′,5′-cyclic- | 0.065 | GO:phosphoric.diester. |
| | | | diester | | AMP | | hydrolase.activity,3′,′-cyclic- |
| | | | hydrolase | | phosphodies- | | AMP.phosphodiesterase. |
| | | | activity | | terase activity | | activity |
| 3.33 | Ubiquitylation- | 5 | xy lulose | 0.027 | transferase | 0.027 | GO:xylulose.biosynthetic. |
| | and- | | biosynthetic | | activity, | | process,transferase.activity, |
| | Sumoylation | | process | | transferring | | transferring.aldehyde.or. |
| | | | | | aldehyde or | | ketonic.groups |
| | | | | | ketonic groups | | |
| 3.33.178 | Unknown | 7 | mitochondrion | 0.302 | ATPase | 0.07 | GO:mitochondrion, |
| | | | | | binding | | ATPase.binding |
| 3.34 | Unknown | 39 | nucleobase- | 0.403 | nucleic acid | 0.376 | GO:nucleobase- |
| | | | containing | | metabolic | | containing.compound. |
| | | | compound | | process | | metabolic.process,nucleic. |
| | | | metabolic | | | | acid.metabolic.process |
| | | | process | | | | |
| 3.35.184 | Transcription- | 4 | heterocyclic | 0.56 | organic cyclic | 0.56 | GO:heterocyclic.compound. |
| | Factors | | compound | | compound | | binding, organic.cyclic. |
| | | | binding | | binding | | compound.binding |
| 3.44 | Golgi | 5 | organelle | 0.4 | bounding | 0.333 | GO:organelle.membrane, |
| | | | membrane | | membrane of | | bounding.membrane.of. |
| | | | | | organelle | | organelle |
| 3.45 | | | glycoside | 0.065 | photoreceptor | 0.065 | GO:glycoside.metabolic. |
| | | | metabolic | | outer segment | | process, |
| | | | process | | | | photoreceptor.outer.segment |

TABLE 1B
The genes within the significant gene modules, listed in Table 1A. (3966 Genes Listed
by: Gene Symbol | Gen 2 Module | Gene Description | RowVariance | Gen 3 Module ||)

ABLIM3 | 2.10 | actin binding LIM protein family member 3 | 0.918107416 | 210.85 || ACTG2 |
2.10 | actin, gamma 2, smooth muscle, enteric | 2.681393002 | 210.86 || ADAMTS9-AS2 | 2.10 |
ADAMTS9 antisense RNA 2 | 0.354316084 | 210.86 || ADGRL3 | 2.10 | adhesion G protein-
coupled receptor L3 | 0.321441803 | 210.85 || AIFIL | 2.10 | allograft inflammatory factor 1-like
1.238551409 | 210.85 || AOC1 | 2.10 | amine oxidase, copper containing 1 | 0.472162092 | 2.10.86 ||
ATP8A1 | 2.10 | ATPase phospholipid transporting 8A1 | 1.060010383 | 210.85 || B4GAT1 | 2.10 |
beta-1,4-glucuronyltransferase 1 | 0.373823148 | 210.86 || BCAM | 2.10 | basal cell adhesion
molecule (Lutheran blood group) | 0.512976091 | 210.86 || BEND7 | 2.10 | BEN domain containing
7 | 0.563042785 | 210.86 || BMP2K | 2.10 | BMP2 inducible kinase | 0.426726609 | 210.85 || BMX
| 2.10 | BMX non-receptor tyrosine kinase | 0.561782814 | 210.86 || C10orf128 | 2.10 | chromosome
10 open reading frame 128 | 0.544142073 | 210.85 || C1QTNF2 | 2.10 | Clq and tumor necrosis
factor related protein 2 | 1.27557369 | 210.85 || C1QTNF3 | 2.10 | C1q and tumor necrosis factor
related protein 3 | 1.779854009 | 210.85 || C2orf88 | 2.10 | chromosome 2 open reading frame 88 |
0.553845983 | 210.86 || CCDC146 | 2.10 | coiled-coil domain containing 146 | 1.196073898 |
210.85 || CEP70 | 2.10 | centrosomal protein 70kDa | 0.341250967 | 210.85 || CHL1 | 2.10 | cell
adhesion molecule LI like | 0.568801659 | 210.85 || CKB | 2.10 | creatine kinase, brain |
0.340419667 | 210.85 || CLDN5 | 2.10 | claudin 5 | 0.67316544 | 210.85 || CNKSR3 | 2.10 |
CNKSR family member 3 | 1.087384668 | 210.85 || CNN1 | 2.10 | calponin 1 | 1.226206206 |
210.86 || CRABP2 | 2.10 | cellular retinoic acid binding protein 2 | 1.179542489 | 2.10.85 ||
CSN1S1 | 2.10 | casein alpha s1 | 11.00008899 | 210.85 || CSRP2 | 2.10 | cysteine and glycine rich
protein 2 | 0.847806183 | 210.85 || CX3CL1 | 2.10 | C-X3-C motif chemokine ligand 1 |
0.583778583 | 210.86 || CYP39A1 | 2.10 | cytochrome P450 family 39 subfamily A member 1 |
0.22957737 | 210.85 || CYP4X1 | 2.10 | cytochrome P450 family 4 subfamily X member 1 |
1.492758688 | 210.85 || DMTN | 2.10 | dematin actin binding protein | 1.067084419 | 2.10.85 ||
DPY19L2 | 2.10 | dpy-19 like 2 (C. elegans) | 0.522178905 | 210.85 || EFHD1 | 2.10 | EF-hand
domain family member D1 | 0.868032029 | 210.85 || EFNA1 | 2.10 | ephrin-A1 | 0.595345339 |
210.85 || EGFL6 | 2.10 | EGF like domain multiple 6 | 2.909166402 | 210.85 || ELOVL7 | 2.10 |
ELOVL fatty acid elongase 7 | 1.099867015 | 210.85 || EMX2 | 2.10 | empty spiracles homeobox 2 |
0.368974286 | 210.86 || FAM118A | 2.10 | family with sequence similarity 118 member A |
0.533069348 | 210.86 || FAM69A | 2.10 | family with sequence similarity 69 member A |
0.75771855 | 210.85 || FASN | 2.10 | fatty acid synthase | 2.156334083 | 210.85 || FBLN2 | 2.10 |
fibulin 2 | 1.910088947 | 210.85 || FBN1 | 2.10 | fibrillin 1 | 0.329511475 | 210.85 || FCGRIB |
2.10 | Fc fragment of IgG receptor Ib | 1.963637721 | 210.86 || GALNT15 | 2.10 | polypeptide N-
acetylgalactosaminyltransferase 15 | 1.404681672 | 210.85 || GDF10 | 2.10 | growth differentiation
factor 10 | 1.037748667 | 210.86 || GFOD1 | 2.10 | glucose-fructose oxidoreductase domain
containing 1 | 0.244455616 | 210.85 || GLIPR1 | 2.10 | GLI pathogenesis related 1 | 0.579992726 |
210.86 || GPC4 | 2.10 | glypican 4 | 0.574899 | 210.86 || GPER1 | 2.10 | G protein-coupled estrogen
receptor 1 | 0.251087757 | 210.86 || GPR146 | 2.10 | G protein-coupled receptor 146 | 0.269563856
| 210.85 || GPRC5B | 2.10 | G protein-coupled receptor class C group 5 member B | 1.059447382 |
210.85 || GYPC | 2.10 | glycophorin C (Gerbich blood group) | 0.231357572 | 210.85 || HEY2 |
2.10 | hes related family bHLH transcription factor with YRPW motif 2 | 0.853286389 | 2.10.86 ||
IMMP2L | 2.10 | inner mitochondrial membrane peptidase subunit 2 | 0.303389131 | 2.10.85 ||
ITGA8 | 2.10 | integrin subunit alpha 8 | 0.490647066 | 210.86 || KCNK6 | 2.10 | potassium two
pore domain channel subfamily K member 6 | 0.280116384 | 210.85 || KL | 2.10 | klotho |
0.456734788 | 210.85 || KNSTRN | 2.10 | kinetochore-localized astrin/SPAG5 binding protein |
0.475362014 | 210.86 || LGI4 | 2.10 | leucine-rich repeat LGI family member 4 | 0.420987386 |
210.85 || LMOD1 | 2.10 | leiomodin 1 | 1.765563041 | 210.86 || LRRK2 | 2.10 | leucine-rich repeat
kinase 2 | 0.38013275 | 210.86 || MGST2 | 2.10 | microsomal glutathione S-transferase 2 |
0.302643467 | 210.85 || MS4A7 | 2.10 | membrane spanning 4-domains A7 | 0.299950563 | 2.10.85
|| MTMR11 | 2.10 | myotubularin related protein 11 | 0.216755965 | 210.86 || MY05C | 2.10 |
myosin VC | 1.284655035 | 210.85 || NAA30 | 2.10 | N(alpha)-acetyltransferase 30, NatC catalytic
subunit | 0.220156535 | 210.85 || NHSL1 | 2.10 | NHS like 1 | 1.237192591 | 210.85 || NPNT | 2.10
| nephronectin | 0.946837793 | 210.85 || NPR1 | 2.10 | natriuretic peptide receptor 1 | 0.469798282 |
210.85 || NPYIR | 2.10 | neuropeptide Y receptor Y1 | 2.188509459 | 210.85 || NR3C2 | 2.10 |
nuclear receptor subfamily 3 group C member 2 | 1.259023048 | 210.86 || NTM | 2.10 | neurotrimin
| 0.724804606 | 210.85 || OSR1 | 2.10 | odd-skipped related transciption factor 1 | 1.020581264 |
210.86 || P4HA3 | 2.10 | prolyl 4-hydroxylase subunit alpha 3 | 0.547012038 | 210.85 || PAMR1 |
2.10 | peptidase domain containing associated with muscle regeneration 1 | 0.659003856 | 2.10.85 ||
PARD3B | 2.10 | par-3 family cell polarity regulator beta | 0.254279079 | 210.85 || PCDH19 | 2.10 |
protocadherin 19 | 0.320377378 | 210.86 || PDE2A | 2.10 | phosphodiesterase 2A | 0.717288025 |
210.85 || PECAM1 | 2.10 | platelet/endothelial cell adhesion molecule 1 | 0.378678836 | 2.10.85 ||
PLCB4 | 2.10 | phospholipase C beta 4 | 1.608628586 | 210.85 || PLP1 | 2.10 | proteolipid protein 1 |
1.048854407 | 210.85 || PPP1R12B | 2.10 | protein phosphatase 1 regulatory subunit 12B |
0.990958928 | 210.86 || PPP1R14A | 2.10 | protein phosphatase 1 regulatory inhibitor subunit 14A |
0.810386166 | 210.85 || PPP2R5A | 2.10 | protein phosphatase 2 regulatory subunit B′, alpha |
0.363039828 | 210.85 || PRR5 | 2.10 | proline rich 5 | 0.404725987 | 210.85 || PRRG3 | 2.10 |
proline rich Gla (G-carboxyglutamic acid) 3 (transmembrane) | 0.45436329 | 210.86 || PXMP2 |
2.10 | peroxisomal membrane protein 2 | 0.295405095 | 210.85 || RAB7B | 2.10 | RAB7B, member
RAS oncogene family | 0.244838109 | 210.86 || RBPMS2 | 2.10 | RNA binding protein with
multiple splicing 2 | 0.22140223 | 210.86 || RPIA | 2.10 | ribose 5-phosphate isomerase A |
0.327943173 | 210.85 || SAMSN1 | 2.10 | SAM domain, SH3 domain and nuclear localization
signals 1 | 0.373414953 | 210.85 || SCAI | 2.10 | suppressor of cancer cell invasion | 0.34960713 |
210.85 || SCAPER | 2.10 | S-phase cyclin A-associated protein in the ER | 0.233490435 | 2.10.86 ||
SCD | 2.10 | stearoyl-CoA desaturase (delta-9-desaturase) | 2.135434836 | 210.85 || SCIN | 2.10 |
scinderin | 0.608164379 | 210.85 || SERPINI1 | 2.10 | serpin peptidase inhibitor, clade I
(neuroserpin), member 1 | 0.827771811 | 210.86 || SH3BGRL2 | 2.10 | SH3 domain binding
glutamate rich protein like 2 | 1.247498167 | 210.85 || SLC38A6 | 2.10 | solute carrier family 38
member 6 | 0.264391916 | 210.85 || SNCG | 2.10 | synuclein gamma | 0.847912157 | 2.10.85 ||
STAT5B | 2.10 | signal transducer and activator of transcription 5B | 0.260382058 | 2.10.85 ||
STK39 | 2.10 | serine/threonine kinase 39 | 0.92253157 | 210.85 || STRADB | 2.10 | STE20-related
kinase adaptor beta | 0.463501365 | 210.85 || TACR1 | 2.10 | tachykinin receptor 1 | 0.927781846 |
210.85 || TBC1D8 | 2.10 | TBC1 domain family member 8 | 0.420615476 | 210.85 || TCEAL7 |
2.10 | transcription elongation factor A like 7 | 0.89570466 | 210.85 || TESC | 2.10 | tescalcin |
0.222983729 | 210.86 || TF | 2.10 | transferrin | 3.804873105 | 210.85 || TIMD4 | 2.10 | T-cell
immunoglobulin and mucin domain containing 4 | 4.181045228 | 210.85 || TLE1 | 2.10 | transducin
like enhancer of split 1 | 0.29581844 | 210.85 || TNFSF8 | 2.10 | tumor necrosis factor superfamily
member 8 | 0.537943722 | 210.85 || TUB | 2.10 | tubby bipartite transcription factor | 0.596297969 |
210.86 || VRK3 | 2.10 | vaccinia related kinase 3 | 0.224945685 | 210.85 || WFS1 | 2.10 | wolframin
ER transmembrane glycoprotein | 0.423762466 | 210.85 || WTIP | 2.10 | Wilms tumor 1 interacting
protein | 0.250272167 | 210.86 || ZFPM2 | 2.10 | zinc finger protein, FOG family member 2 |
1.097936248 | 210.86 || ABLIM3 | 210.85 || actin binding LIM protein family member 3 |
0.918107416 | 210.85 || ADGRL3 | 210.85 || adhesion G protein-coupled receptor L3 | 0.321441803
| 210.85 || AIFIL | 210.85 || allograft inflammatory factor 1-like | 1.238551409 | 210.85 || ATP8A1
| 210.85 || ATPase phospholipid transporting 8A1 | 1.060010383 | 210.85 || BMP2K | 2.10.85 |
BMP2 inducible kinase | 0.426726609 | 210.85 || C10orf128 | 210.85 | chromosome 10 open
reading frame 128 | 0.544142073 | 210.85 || C1QTNF2 | 210.85 | C1q and tumor necrosis factor
related protein 2 | 1.27557369 | 210.85 || C1QTNF3 | 210.85 | C1q and tumor necrosis factor
related protein 3 | 1.779854009 | 210.85 || CCDC146 | 210.85 | coiled-coil domain containing 146 |
1.196073898 | 210.85 || CEP70 | 210.85 || centrosomal protein 70kDa | 0.341250967 | 2.10.85 ||
CHL1 | 210.85 || cell adhesion molecule LI like | 0.568801659 | 210.85 || CKB | 210.85 | creatine
kinase, brain | 0.340419667 | 210.85 || CLDN5 | 210.85 | claudin 5 | 0.67316544 | 2.10.85 ||
CNKSR3 | 210.85 || CNKSR family member 3 | 1.087384668 | 210.85 || CRABP2 | 2.10.85 |
cellular retinoic acid binding protein 2 | 1.179542489 | 210.85 || CSN1S1 | 210.85 | casein alpha s1
| 11.00008899 | 210.85 || CSRP2 | 210.85 | cysteine and glycine rich protein 2 | 0.847806183 |
210.85 || CYP39A1 | 210.85 | cytochrome P450 family 39 subfamily A member 1 | 0.22957737 |
210.85 || CYP4X1 | 210.85 | cytochrome P450 family 4 subfamily X member 1 | 1.492758688 |
210.85 || DMTN | 210.85 | dematin actin binding protein | 1.067084419 | 210.85 || DPY19L2 |
210.85 || dpy-19 like 2 (C. elegans) | 0.522178905 | 210.85 || EFHD1 | 210.85 | EF-hand domain
family member D1 | 0.868032029 | 210.85 || EFNA1 | 210.85 | ephrin-A1 | 0.595345339 | 2.10.85 ||
EGFL6 | 210.85 | EGF like domain multiple 6 | 2.909166402 | 210.85 || ELOVL7 | 2.10.85 |
ELOVL fatty acid elongase 7 | 1.099867015 | 210.85 | FAM69A | 210.85 | family with sequence
similarity 69 member A | 0.75771855 | 210.85 || FASN | 210.85 | fatty acid synthase | 2.156334083
| 210.85 || FBLN2 | 210.85 | fibulin 2 | 1.910088947 | 210.85 | FBN1 | 210.85 | fibrillin 1 |
0.329511475 | 210.85 || GALNT15 | 210.85 | polypeptide N-acetylgalactosaminyltransferase 15 |
1.404681672 | 210.85 || GFOD1 | 210.85 | glucose-fructose oxidoreductase domain containing 1 |
0.244455616 | 210.85 || GPR146 | 210.85 | G protein-coupled receptor 146 | 0.269563856 | 2.10.85
|| GPRC5B | 210.85 | G protein-coupled receptor class C group 5 member B | 1.059447382 |
210.85 || GYPC | 210.85 | glycophorin C (Gerbich blood group) | 0.231357572 | 2.10.85 ||
IMMP2L | 210.85 | inner mitochondrial membrane peptidase subunit 2 | 0.303389131 | 2.10.85 ||
KCNK6 | 210.85 | potassium two pore domain channel subfamily K member 6 | 0.280116384 |
210.85 || KL | 210.85 | klotho | 0.456734788 | 210.85 || LGI4 | 210.85 | leucine-rich repeat LGI
family member 4 | 0.420987386 | 210.85 || MGST2 | 210.85 | microsomal glutathione S-transferase
2 | 0.302643467 | 210.85 || MS4A7 | 210.85 | membrane spanning 4-domains A7 | 0.299950563 |
210.85 || MYO5C | 210.85 | myosin VC | 1.284655035 | 210.85 | NAA30 | 210.85 | N(alpha)-
acetyltransferase 30, NatC catalytic subunit | 0.220156535 | 210.85 || NHSL1 | 210.85|| NHS like 1
| 1.237192591 | 210.85 || NPNT | 210.85 | nephronectin | 0.946837793 | 210.85 || NPR1 | 2.10.85 |
natriuretic peptide receptor 1 | 0.469798282 | 210.85 || NPYIR | 210.85 | neuropeptide Y receptor
Y1 | 2.188509459 | 210.85 || NTM | 210.85 | neurotrimin | 0.724804606 | 210.85 || P4HA3 |
210.85 | prolyl 4-hydroxylase subunit alpha 3 | 0.547012038 | 210.85 | PAMR1 | 2.10.85 |
peptidase domain containing associated with muscle regeneration 1 | 0.659003856 | 2.10.85 ||
PARD3B | 210.85 | par-3 family cell polarity regulator beta | 0.254279079 | 210.85 || PDE2A |
210.85 || phosphodiesterase 2A | 0.717288025 | 210.85 || PECAM1 | 210.85 | platelet/endothelial
cell adhesion molecule 1 | 0.378678836 | 210.85 || PLCB4 | 210.85 | phospholipase C beta 4 |
1.608628586 | 210.85 || PLP1 | 210.85 | proteolipid protein 1 | 1.048854407 | 210.85 | PPP1R14A
| 210.85 | protein phosphatase 1 regulatory inhibitor subunit 14A | 0.810386166 | 2.10.85 ||
PPP2R5A | 210.85 | protein phosphatase 2 regulatory subunit B′, alpha | 0.363039828 | 2.10.85 ||
PRR5 | 210.85 | proline rich 5 | 0.404725987 | 210.85 || PXMP2 | 210.85 | peroxisomal membrane
protein 2 | 0.295405095 | 210.85 || RPIA | 210.85 | ribose 5-phosphate isomerase A | 0.327943173 |
210.85 || SAMSN1 | 210.85 | SAM domain, SH3 domain and nuclear localization signals 1 |
0.373414953 | 210.85 || SCAI | 210.85 | suppressor of cancer cell invasion | 0.34960713 | 2.10.85 ||
SCD | 210.85 | stearoyl-CoA desaturase (delta-9-desaturase) | 2.135434836 | 210.85 || SCIN |
210.85 | scinderin | 0.608164379 | 210.85 || SH3BGRL2 | 210.85 | SH3 domain binding glutamate
rich protein like 2 | 1.247498167 | 210.85 || SLC38A6 | 210.85 | solute carrier family 38 member 6
| 0.264391916 | 210.85 || SNCG | 210.85 | synuclein gamma | 0.847912157 | 210.85 || STAT5B |
210.85 | signal transducer and activator of transcription 5B | 0.260382058 | 210.85 || STK39 |
210.85 | serine/threonine kinase 39 | 0.92253157 | 210.85 || STRADB | 210.85 | STE20-related
kinase adaptor beta | 0.463501365 | 210.85 || TACR1 | 210.85 | tachykinin receptor 1 |
0.927781846 | 210.85 || TBC1D8 | 210.85 | TBC1 domain family member 8 | 0.420615476 |
210.85 || TCEAL7 | 210.85 | transcription elongation factor A like 7 | 0.89570466 | 210.85 || TF |
210.85 | transferrin | 3.804873105 | 210.85 || TIMD4 | 210.85 | T-cell immunoglobulin and mucin
domain containing 4 | 4.181045228 | 210.85 || TLE1 | 210.85 | transducin like enhancer of split 1 |
0.29581844 | 210.85 || TNFSF8 | 210.85 | tumor necrosis factor superfamily member 8 |
0.537943722 | 210.85 || VRK3 | 210.85 | vaccinia related kinase 3 | 0.224945685 | 210.85 || WFS1
| 210.85 | wolframin ER transmembrane glycoprotein | 0.423762466 | 210.85 || AASDH | 2.11 |
aminoadipate-semialdehyde dehydrogenase | 0.460028186 | - || ABAT | 2.11 | 4-aminobutyrate
aminotransferase | 0.459696688 | 2.11.91 || ABCD3 | 2.11 | ATP binding cassette subfamily D
member 3 | 0.303894131 | 2.11.88 || ABI2 | 2.11 | abl-interactor 2 | 0.750557435 | 2.11.93 || ABI3BP
| 2.11 | ABI family member 3 binding protein | 0.380939275 | 2.11.93 || ACADSB | 2.11 | acyl-CoA
dehydrogenase, short/branched chain | 0.448305681 | 2.11.87 || ACOT7 | 2.11 | acyl-CoA
thioesterase 7 | 1.191420448 | 2.11.88 || ACOX2 | 2.11 | acyl-CoA oxidase 2, branched chain |
0.589616328 | 2.11.87 || ADAM28 | 2.11 | ADAM metallopeptidase domain 28 | 4.175760629 |
2.11.94 || ADAMTS15 | 2.11 | ADAM metallopeptidase with thrombospondin type 1 motif 15 |
0.961050341 | 2.11.94 || ADAMTS5 | 2.11 | ADAM metallopeptidase with thrombospondin type 1
motif 5 | 0.787279475 | 2.11.91 || ADCY3 | 2.11 | adenylate cyclase 3 | 0.370864487 | 2.11.94 ||
ADCY9 | 2.11 | adenylate cyclase 9 | 0.329183235 | 2.11.94 || ADD3 | 2.11 | adducin 3 |
0.607985917 | 2.11.87 || AIG1 | 2.11 | androgen-induced 1 | 1.007814018 | 2.11.95 || AKRIA1 | 2.11
| aldo-keto reductase family 1, member Al (aldehyde reductase) | 0.266734234 | 2.11.88 ||
ALDH18A1 | 2.11 | aldehyde dehydrogenase 18 family member Al | 0.317919461 | 2.11.90 ||
ALDH3 A2 | 2.11 | aldehyde dehydrogenase 3 family member A2 | 0.47984049 | 2.11.95 || AMIGO2
| 2.11 | adhesion molecule with Ig-like domain 2 | 0.814772914 | 2.11.87 || AMOTL1 | 2.11 |
angiomotin like 1 | 0.402363786 | 2.11.95 || ANKRD36B | 2.11 | ankyrin repeat domain 36B |
0.705201592 | 2.11.88 || AOX1 | 2.11 | aldehyde oxidase 1 | 1.679142604 | 2.11.94 || AQP1 | 2.11 |
aquaporin 1 (Colton blood group) | 0.523004709 | - || ARHGAP25 | 2.11 | Rho GTPase activating
protein 25 | 0.653693319 | 2.11.94 || ARHGEF12 | 2.11 | Rho guanine nucleotide exchange factor 12
| 0.253338823 | 2.11.91 || ARNTL2 | 2.11 | aryl hydrocarbon receptor nuclear translocator like 2 |
0.494590322 | 2.11.96 || ARSK | 2.11 | arylsulfatase family member K | 0.245695698 | 2.11.94 ||
ATE1 | 2.11 | arginyltransferase 1 | 0.515901455 | - || ATL1 | 2.11 | atlastin GTPase 1 | 0.398278492
| 2.11.90 || ATOH8 | 2.11 | atonal bHLH transcription factor 8 | 0.342741943 | 2.11.92 || ATP2A3 |
2.11 | ATPase sarcoplasmic/endoplasmic reticulum Ca2+ transporting 3 | 0.539887479 | - ||
B3GNT5 | 2.11 | UDP-GlcNAc:betaGal beta-1,3-N-acetylglucosaminyltransferase 5 | 0.496099263 |
2.11.94 || BAX | 2.11 | BCL2-associated X protein | 0.286362311 | 2.11.88 || BBS10 | 2.11 | Bardet-
Biedl syndrome 10 | 0.259991801 | 2.11.92 || BBX | 2.11 | bobby sox homolog (Drosophila) |
0.309003234 | - || BIRC7 | 2.11 | baculoviral IAP repeat containing 7 | 0.364114946 | 2.11.89 ||
BMP4 | 2.11 | bone morphogenetic protein 4 | 1.275566837 | 2.11.94 || BMPRIA | 2.11 | bone
morphogenetic protein receptor type 1A | 0.613503708 | 2.11.87 || BNC2 | 2.11 | basonuclin 2 |
0.902943933 | 2.11.91 || BRAP | 2.11 | BRCA1 associated protein | 0.223831083 | - || BTN2A2 |
2.11 | butyrophilin subfamily 2 member A2 | 0.475629665 | 2.11.94 || C11orf96 | 2.11 | chromosome
11 open reading frame 96 | 1.09996502 | 2.11.95 || C12orf29 | 2.11 | chromosome 12 open reading
frame 29 | 0.311949406 | 2.11.95 || C15orf48 | 2.11 | chromosome 15 open reading frame 48 |
9.470344982 | 2.11.94 || C17orf80 | 2.11 | chromosome 17 open reading frame 80 | 0.268113993 |
2.11.95 || C19orf68 | 2.11 | chromosome 19 open reading frame 68 | 0.454827829 | 2.11.88 ||
C22orf39 | 2.11 | chromosome 22 open reading frame 39 | 0.215432391 | 2.11.91 || C7 | 2.11 |
complement component 7 | 5.235794833 | 2.11.96 || C7orf50 | 2.11 | chromosome 7 open reading
frame 50 | 0.563034422 | 2.11.89 || C7orf55 | 2.11 | chromosome 7 open reading frame 55 |
0.668742837 | 2.11.87 || CADPS2 | 2.11 | Ca2+ dependent secretion activator 2 | 0.573610167 |
2.11.90 || CALD1 | 2.11 | caldesmon 1 | 0.418718877 | 2.11.91 || CAMLG | 2.11 | calcium
modulating ligand | 0.285590664 | 2.11.96 || CASP6 | 2.11 | caspase 6 | 0.798729681 | 2.11.88 ||
CASR | 2.11 | calcium sensing receptor | 0.229062214 | 2.11.89 || CAV1 | 2.11 | caveolin 1 |
0.398398443 | 2.11.95 || CBR1 | 2.11 | carbonyl reductase 1 | 0.34570911 | 2.11.88 || CCAR1 | 2.11 |
cell division cycle and apoptosis regulator 1 | 0.31308275 | 2.11.88 || CCDC3 | 2.11 | coiled-coil
domain containing 3 | 1.522689954 | 2.11.95 || CCSAP | 2.11 | centriole, cilia and spindle associated
protein | 0.263171876 | 2.11.95 || CD248 | 2.11 | CD248 molecule | 0.68823608 | 2.11.91 || CD37 |
2.11 | CD37 molecule | 1.133372643 | 2.11.94 || CD38 | 2.11 | CD38 molecule | 2.150406121 |
2.11.88 || CD53 | 2.11 | CD53 molecule | 0.50285979 | 2.11.94 || CDC14B | 2.11 | cell division cycle
14B | 0.606848097 | 2.11.95 || CDC37L1 | 2.11 | cell division cycle 37-like 1 | 0.231469743 |
2.11.87 || CDC42BPA | 2.11 | CDC42 binding protein kinase alpha | 0.998600128 | 2.11.95 ||
CDKN3 | 2.11 | cyclin-dependent kinase inhibitor 3 | 0.895603292 | 2.11.90 || CDO1 | 2.11 |
cysteine dioxygenase type 1 | 0.759959385 | 2.11.95 || CEBPA-AS1 | 2.11 | CEBPA antisense RNA
1 (head to head) | 0.219803491 | 2.11.88 || CEMP1 | 2.11 | cementum protein 1 | 0.327109293 |
2.11.88 || CEP126 | 2.11 | centrosomal protein 126kDa | 1.892868245 | 2.11.88 || CEP164 | 2.11 |
centrosomal protein 164kDa | 0.955611752 | 2.11.88 || CEP290 | 2.11 | centrosomal protein 290kDa
| 0.263768045 | 2.11.87 || CEP57 | 2.11 | centrosomal protein 57kDa | 0.366056851 | 2.11.91 ||
CEP85 | 2.11 | centrosomal protein 85kDa | 0.383549817 | 2.11.93 || CFH | 2.11 | complement factor
H | 0.713023953 | 2.11.96 || CILP | 2.11 | cartilage intermediate layer protein | 4.923465697 |
2.11.92 || CLASP1 | 2.11 | cytoplasmic linker associated protein 1 | 0.271094193 | 2.11.93 ||
CLOCK | 2.11 | clock circadian regulator | 0.293393216 | 2.11.87 || CMTM4 | 2.11 | CKLF like
MARVEL transmembrane domain containing 4 | 0.314085805 | 2.11.94 || COG6 | 2.11 | component
of oligomeric golgi complex 6 | 0.286369934 | 2.11.90 || COL27A1 | 2.11 | collagen type XXVII
alpha 1 | 0.892869302 | 2.11.87 || COL7A1 | 2.11 | collagen type VII alpha 1 | 0.368213732 |
2.11.88 || CPA3 | 2.11 | carboxypeptidase A3 | 1.668080728 | 2.11.90 || CPE | 2.11 |
carboxypeptidase E | 1.901786183 | 2.11.96 || CPVL | 2.11 | carboxypeptidase, vitellogenic like |
0.287188234 | 2.11.94 || CPXM2 | 2.11 | carboxypeptidase X (M14 family), member 2 |
0.709943449 | - || CSF2RA | 2.11 | colony stimulating factor 2 receptor alpha subunit | 0.606462659
| 2.11.95 || CTGF | 2.11 | connective tissue growth factor | 0.827377472 | 2.11.92 || CTSH | 2.11 |
cathepsin H | 0.62741109 | 2.11.94 || CTSV | 2.11 | cathepsin V | 0.291857737 | 2.11.95 || CTU2 |
2.11 | cytosolic thiouridylase subunit 2 homolog (S. pombe) | 0.382667246 | 2.11.95 || CWC27 |
2.11 | CWC27 spliceosome associated protein homolog | 0.243405515 | 2.11.87 || CX3CR1 | 2.11 |
chemokine (C-X3-C motif) receptor 1 | 1.892071056 | - || CXCR4 | 2.11 | chemokine (C-X-C motif)
receptor 4 | 1.269143152 | 2.11.94 || CYB5R4 | 2.11 | cytochrome b5 reductase 4 | 0.392472681 |
2.11.94 || CYP26B1 | 2.11 | cytochrome P450 family 26 subfamily B member 1 | 2.559696531 |
2.11.92 || CYR61 | 2.11 | cysteine rich angiogenic inducer 61 | 2.036162468 | 2.11.95 || CYTH4 |
2.11 | cytohesin 4 | 0.614764481 | 2.11.94 || DAAM1 | 2.11 | dishevelled associated activator of
morphogenesis 1 | 0.471280572 | 2.11.87 || DAB2 | 2.11 | Dab, mitogen-responsive phosphoprotein,
homolog 2 (Drosophila) | 0.438747419 | 2.11.93 || DCBLD1 | 2.11 | discoidin, CUB and LCCL
domain containing 1 | 0.229067276 | 2.11.92 || DCBLD2 | 2.11 | discoidin, CUB and LCCL domain
containing 2 | 0.444713707 | 2.11.93 || DCLK2 | 2.11 | doublecortin like kinase 2 | 0.511160655 |
2.11.87 || DDR2 | 2.11 | discoidin domain receptor tyrosine kinase 2 | 0.942052657 | 2.11.87 ||
DDX10 | 2.11 | DEAD-box helicase 10 | 0.233683198 | 2.11.92 || DDX54 | 2.11 | DEAD-box
helicase 54 | 0.613719873 | 2.11.88 || DESI1 | 2.11 | desumoylating isopeptidase 1 | 0.404636144 |
2.11.96 || DHRS4-AS1 | 2.11 | DHRS4 antisense RNA 1 | 0.23517016 | 2.11.95 || DHX33 | 2.11 |
DEAH-box helicase 33 | 0.378118657 | 2.11.88 || DIAPH2 | 2.11 | diaphanous related formin 2 |
0.275314812 | 2.11.91 || DIO2 | 2.11 | deiodinase, iodothyronine, type II | 3.958061425 | 2.11.90 ||
DIP2C | 2.11 | disco interacting protein 2 homolog C | 0.519361999 | 2.11.91 || DIXDC1 | 2.11 |
DIX domain containing 1 | 0.759440001 | 2.11.87 || DLC1 | 2.11 | DLC1 Rho GTPase activating
protein | 1.330005516 | 2.11.95 || DMXL2 | 2.11 | Dmx like 2 | 0.392168413 | 2.11.95 || DNAJB9 |
2.11 | DnaJ heat shock protein family (Hsp40) member B9 | 0.375758976 | 2.11.88 || DOHH | 2.11 |
deoxyhypusine hydroxylase/monooxygenase | 0.423163335 | 2.11.94 || DPP4 | 2.11 | dipeptidyl
peptidase 4 | 1.0578736 | 2.11.88 || DPY19L3 | 2.11 | dpy-19 like 3 (C. elegans) | 0.596164066 |
2.11.93 || DPY19L4 | 2.11 | dpy-19 like 4 (C. elegans) | 0.543100176 | 2.11.93 || DSPP | 2.11 |
dentin sialophosphoprotein | 0.260053984 | 2.11.93 || DYNC2H1 | 2.11 | dynein cytoplasmic 2
heavy chain 1 | 0.277564418 | 2.11.90 || DYNC2LI1 | 2.11 | dynein cytoplasmic 2 light intermediate
chain 1 | 0.626974122 | 2.11.87 || DZIPIL | 2.11 | DAZ interacting zinc finger protein 1 like |
0.475805534 | 2.11.94 || EBF1 | 2.11 | early B-cell factor 1 | 0.937984168 | 2.11.87 || EBF2 | 2.11 |
early B-cell factor 2 | 3.351821547 | 2.11.87 || EBPL | 2.11 | emopamil binding protein like |
0.556331861 | 2.11.91 || EDEM2 | 2.11 | ER degradation enhancer, mannosidase alpha-like 2 |
0.286374549 | 2.11.88 || EDN1 | 2.11 | endothelin 1 | 0.394131287 | 2.11.94 || EFCAB7 | 2.11 | EF-
hand calcium binding domain 7 | 0.460111073 | 2.11.93 || EFS | 2.11 | embryonal Fyn-associated
substrate | 0.591158755 | 2.11.87 || EIF5A | 2.11 | eukaryotic translation initiation factor 5A |
5.16344018 | 2.11.95 || EIF5B | 2.11 | eukaryotic translation initiation factor 5B | 0.310760685 |
2.11.89 || EMSY | 2.11 | EMSY, BRCA2 interacting transcriptional repressor | 0.363617224 |
2.11.89 || EMX2OS | 2.11 | EMX2 opposite strand/antisense RNA | 0.226374468 | 2.11.94 ||
EPB41L2 | 2.11 | erythrocyte membrane protein band 4.1-like 2 | 0.261723475 | 2.11.92 || EPHA4 |
2.11 | EPH receptor A4 | 0.51724409 | 2.11.94 || EPS8 | 2.11 | epidermal growth factor receptor
pathway substrate 8 | 0.248989987 | - || ERAP2 | 2.11 | endoplasmic reticulum aminopeptidase 2 |
5.584573823 | 2.11.95 || ERGIC1 | 2.11 | endoplasmic reticulum-golgi intermediate compartment 1 |
0.22486897 | 2.11.94 || ETNK1 | 2.11 | ethanolamine kinase 1 | 0.226392511 | - || EVAIC | 2.11 |
eva-1 homolog C (C. elegans) | 1.030366337 | 2.11.96 || EVI2A | 2.11 | ecotropic viral integration
site 2A | 0.468198362 | 2.11.94 || EXT1 | 2.11 | exostosin glycosyltransferase 1 | 0.376437861 |
2.11.93 || EYA4 | 2.11 | EYA transcriptional coactivator and phosphatase 4 | 0.759943865 | 2.11.87
|| FAM107A | 2.11 | family with sequence similarity 107 member A | 1.51719541 | 2.11.94 ||
FAM122B | 2.11 | family with sequence similarity 122B | 0.290955827 | - || FAM179B | 2.11 |
family with sequence similarity 179 member B | 0.306610698 | 2.11.93 || FAM210B | 2.11 | family
with sequence similarity 210 member B | 0.277177641 | 2.11.87 || FAM228B | 2.11 | family with
sequence similarity 228 member B | 0.349991862 | 2.11.96 || FAM92A1 | 2.11 | family with
sequence similarity 92 member Al | 0.634098775 | 2.11.91 || FANCL | 2.11 | Fanconi anemia
complementation group L | 0.259473729 | 2.11.93 || FBLN5 | 2.11 | fibulin 5 | 1.139777302 |
2.11.96 || FBXL17 | 2.11 | F-box and leucine-rich repeat protein 17 | 0.321221777 | 2.11.87 ||
FBXO31 | 2.11 | F-box protein 31 | 0.378024398 | - || FCGR2A | 2.11 | Fc fragment of IgG receptor
IIa | 0.53215917 | 2.11.94 || FCGR2C | 2.11 | Fc fragment of IgG receptor IIc (gene/pseudogene) |
1.154315254 | 2.11.94 || FERMT3 | 2.11 | fermitin family member 3 | 0.485470904 | 2.11.94 || FEZ1
| 2.11 | fasciculation and elongation protein zeta 1 | 1.316610022 | 2.11.94 || FGF13 | 2.11 |
fibroblast growth factor 13 | 0.987877209 | 2.11.87 || FGF18 | 2.11 | fibroblast growth factor 18 |
0.690756108 | 2.11.94 || FIGN | 2.11 | fidgetin | 1.045218876 | 2.11.88 || FKBP7 | 2.11 | FK506
binding protein 7 | 0.484285465 | 2.11.90 || FKBP9 | 2.11 | FK506 binding protein 9 | 0.41759953 | -
|| FLJ20021 | 2.11 | uncharacterized LOC90024 | 0.284145227 | 2.11.93 || FLVCR2 | 2.11 | feline
leukemia virus subgroup C cellular receptor family member 2 | 0.587439889 | 2.11.94 || FNDC3B |
2.11 | fibronectin type III domain containing 3B | 0.227687911 | 2.11.93 || FNDC4 | 2.11 |
fibronectin type III domain containing 4 | 0.4953917 | 2.11.87 || FOCAD | 2.11 | focadhesin |
0.268845132 | 2.11.95 || FPR1 | 2.11 | formyl peptide receptor 1 | 0.886015891 | 2.11.93 || FUT5 |
2.11 | fucosyltransferase 5 | 0.385385121 | 2.11.93 || FYN | 2.11 | FYN proto-oncogene, Src family
tyrosine kinase | 0.285535416 | 2.11.87 || GABRB2 | 2.11 | gamma-aminobutyric acid type A
receptor beta2 subunit | 5.135305883 | 2.11.94 || GALNT1 | 2.11 | polypeptide N-
acetylgalactosaminyltransferase 1 | 0.256250248 | 2.11.90 || GAREM1 | 2.11 | GRB2 associated
regulator of MAPKI subtype 1 | 1.00034877 | 2.11.93 || GEM | 2.11 | GTP binding protein
overexpressed in skeletal muscle | 0.619526415 | 2.11.95 || GLDN | 2.11 | gliomedin | 2.746948959 |
2.11.92 || GLPIR | 2.11 | glucagon like peptide 1 receptor | 0.286795346 | 2.11.87 || GLT8D2 | 2.11 |
glycosyltransferase 8 domain containing 2 | 0.75143036 | 2.11.90 || GMFG | 2.11 | glia maturation
factor gamma | 0.45780896 | 2.11.93 || GMIP | 2.11 | GEM interacting protein | 0.715174351 |
2.11.94 || GNA11 | 2.11 | G protein subunit alpha 11 | 0.295970179 | 2.11.91 || GNA15 | 2.11 | G
protein subunit alpha 15 | 0.448280239 | 2.11.95 || GNG12 | 2.11 | G protein subunit gamma 12 |
0.309818361 | 2.11.91 || GOLGA2 | 2.11 | golgin A2 | 0.458222426 | 2.11.93 || GOLIM4 | 2.11 |
golgi integral membrane protein 4 | 0.322114154 | 2.11.93 || GPATCH1 | 2.11 | G-patch domain
containing 1 | 0.219040373 | 2.11.96 || GPATCH4 | 2.11 | G-patch domain containing 4 |
0.31136234 | 2.11.89 || GPHN | 2.11 | gephyrin | 0.379120059 | 2.11.87 || GPR183 | 2.11 | G protein-
coupled receptor 183 | 1.160733904 | 2.11.94 || GPR65 | 2.11 | G protein-coupled receptor 65 |
0.526628376 | 2.11.87 || GPSM2 | 2.11 | G-protein signaling modulator 2 | 0.564926901 | 2.11.91 ||
GPX8 | 2.11 | glutathione peroxidase 8 (putative) | 0.84832034 | 2.11.90 || GSAP | 2.11 | gamma-
secretase activating protein | 0.663756338 | 2.11.94 || GSTT1 | 2.11 | glutathione S-transferase theta
1 | 1.747692966 | 2.11.91 || GTPBP2 | 2.11 | GTP binding protein 2 | 1.042769987 | 2.11.88 ||
GXYLT2 | 2.11 | glucoside xylosyltransferase 2 | 0.737361809 | 2.11.94 || HAMP | 2.11 | hepcidin
antimicrobial peptide | 0.284354134 | 2.11.91 || HAUS6 | 2.11 | HAUS augmin like complex subunit
6 | 0.560054086 | 2.11.93 || HCLS1 | 2.11 | hematopoietic cell-specific Lyn substrate 1 |
0.522189768 | 2.11.94 || HDAC9 | 2.11 | histone deacetylase 9 | 0.408359818 | 2.11.94 || HEATR5A
| 2.11 | HEAT repeat containing 5A | 0.352183612 | - || HES1 | 2.11 | hes family bHLH transcription
factor 1 | 0.693985311 | 2.11.87 || HEY1 | 2.11 | hes related family bHLH transcription factor with
YRPW motif 1 | 0.458956865 | 2.11.94 || HLA-DRB6 | 2.11 | major histocompatibility complex,
class II, DR beta 6 (pseudogene) | 0.63924165 | 2.11.94 || HMGA1 | 2.11 | high mobility group AT-
hook 1 | 0.760994377 | 2.11.95 || HOXA5 | 2.11 | homeobox A5 | 0.886096098 | 2.11.93 || HOXD8 |
2.11 | homeobox D8 | 0.399510556 | 2.11.94 || HS2ST1 | 2.11 | heparan sulfate 2-O-sulfotransferase
1 | 0.314728488 | - || HSPA5 | 2.11 | heat shock protein family A (Hsp70) member 5 | 0.255106633 |
2.11.95 || IBSP | 2.11 | integrin binding sialoprotein | 0.255171768 | 2.11.87 || ICAM3 | 2.11 |
intercellular adhesion molecule 3 | 1.221039619 | - || IFT80 | 2.11 | intraflagellar transport 80 |
0.411958681 | 2.11.93 || IGDCC4 | 2.11 | immunoglobulin superfamily, DCC subclass, member 4 |
1.34717997 | 2.11.91 || IL18BP | 2.11 | interleukin 18 binding protein | 1.786824388 | 2.11.94 ||
IMPACT | 2.11 | impact RWD domain protein | 0.473801861 | - || INSR | 2.11 | insulin receptor |
1.130743908 | 2.11.88 || INTS2 | 2.11 | integrator complex subunit 2 | 0.310307903 | 2.11.88 ||
IRAKIBP1 | 2.11 | interleukin 1 receptor associated kinase 1 binding protein 1 | 0.384567859 |
2.11.88 || IRS1 | 2.11 | insulin receptor substrate 1 | 1.206137104 | 2.11.94 || ISYNA1 | 2.11 |
inositol-3-phosphate synthase 1 | 0.467806734 | 2.11.88 || ITPR2 | 2.11 | inositol 1,4,5-trisphosphate
receptor type 2 | 0.577070542 | 2.11.88 || IVNS1ABP | 2.11 | influenza virus NS1A binding protein |
0.302793942 | 2.11.88 || JPX | 2.11 | JPX transcript, XIST activator (non-protein coding) |
0.780111483 | 2.11.87 || KBTBD6 | 2.11 | kelch repeat and BTB domain containing 6 | 0.412701021
| 2.11.94 || KCNF1 | 2.11 | potassium voltage-gated channel modifier subfamily F member 1 |
0.22645375 | 2.11.89 || KCNJ2 | 2.11 | potassium voltage-gated channel subfamily J member 2 |
0.732493091 | 2.11.93 || KCNT2 | 2.11 | potassium sodium-activated channel subfamily T member
2 | 1.179764931 | 2.11.87 || KCTD1 | 2.11 | potassium channel tetramerization domain containing 1 |
0.4321372 | 2.11.94 || KCTD3 | 2.11 | potassium channel tetramerization domain containing 3 |
0.745709664 | 2.11.87 || KDELC2 | 2.11 | KDEL motif containing 2 | 0.344744974 | 2.11.91 ||
KIRREL | 2.11 | kin of IRRE like (Drosophila) | 0.51901249 | - || KITLG | 2.11 | KIT ligand |
1.097244876 | 2.11.91 || KIZ | 2.11 | kizuna centrosomal protein | 0.311161849 | 2.11.95 || KLHL20
| 2.11 | kelch like family member 20 | 0.472326564 | 2.11.88 || KLHL42 | 2.11 | kelch like family
member 42 | 0.353682523 | 2.11.95 || KLHL6 | 2.11 | kelch like family member 6 | 1.730773716 |
2.11.94 || KLHL8 | 2.11 | kelch like family member 8 | 0.377497362 | 2.11.88 || KLK15 | 2.11 |
kallikrein related peptidase 15 | 0.583177926 | 2.11.88 || KMT5A | 2.11 | lysine methyltransferase
5A | 0.451503802 | 2.11.87 || KPNA5 | 2.11 | karyopherin subunit alpha 5 | 0.471170469 | 2.11.88 ||
LACTB | 2.11 | lactamase beta | 0.523962336 | 2.11.94 || LAMP2 | 2.11 | lysosomal associated
membrane protein 2 | 0.317829107 | 2.11.88 || LAMTOR4 | 2.11 | late endosomal/lysosomal
adaptor, MAPK and MTOR activator 4 | 0.21450644 | 2.11.88 || LAPTM4B | 2.11 | lysosomal
protein transmembrane 4 beta | 0.602776882 | 2.11.91 || LARP6 | 2.11 | La ribonucleoprotein
domain family member 6 | 0.800145084 | 2.11.95 || LDOC1 | 2.11 | leucine zipper, down-regulated
in cancer 1 | 0.429951968 | 2.11.95 || LEPROTL1 | 2.11 | leptin receptor overlapping transcript-like
1 | 0.369399864 | 2.11.87 || LGALS9 | 2.11 | lectin, galactoside-binding, soluble, 9 | 0.513488433 |
2.11.94 || LIMA1 | 2.11 | LIM domain and actin binding 1 | 0.25014224 | 2.11.95 || LIMCH1 | 2.11 |
LIM and calponin homology domains 1 | 1.258866277 | 2.11.87 || LINC01139 | 2.11 | long
intergenic non-protein coding RNA 1139 | 0.581493857 | 2.11.96 || LIPT1 | 2.11 | lipoyltransferase
1 | 0.344910038 | 2.11.91 || LOC101927027 | 2.11 | uncharacterized LOC101927027 | 0.275757716
| 2.11.87 || LOC146880 | 2.11 | Rho GTPase activating protein 27 pseudogene | 0.51948776 |
2.11.88 || LOC400043 | 2.11 | uncharacterized LOC400043 | 0.328282968 | 2.11.93 || LPAR1 | 2.11 |
lysophosphatidic acid receptor 1 | 0.310764182 | 2.11.95 || LPXN | 2.11 | leupaxin | 0.914802675 |
2.11.94 || LYN | 2.11 | LYN proto-oncogene, Src family tyrosine kinase | 0.356572728 | 2.11.95 ||
LYRM5 | 2.11 | LYR motif containing 5 | 0.276852304 | 2.11.87 || LYRM7 | 2.11 | LYR motif
containing 7 | 0.547635505 | 2.11.88 || LYZ | 2.11 | lysozyme | 0.298216054 | 2.11.87 || MAB21L2 |
2.11 | mab-21-like 2 (C. elegans) | 4.062089165 | 2.11.96 || MAGEH1 | 2.11 | MAGE family
member H1 | 0.217957682 | 2.11.91 || MANIC1 | 2.11 | mannosidase alpha class 1C member 1 |
0.702672171 | 2.11.95 || MAOB | 2.11 | monoamine oxidase B | 1.976978543 | 2.11.95 || MAP2K1 |
2.11 | mitogen-activated protein kinase kinase 1 | 0.243976669 | 2.11.90 || MAP9 | 2.11 |
microtubule associated protein 9 | 1.228570929 | 2.11.87 || MAPKBP1 | 2.11 | mitogen-activated
protein kinase binding protein 1 | 0.389266249 | 2.11.95 || MATK | 2.11 | megakaryocyte-associated
tyrosine kinase | 1.377160924 | 2.11.87 || MBP | 2.11 | myelin basic protein | 0.578176871 | 2.11.96
|| MIE3 | 2.11 | malic enzyme 3, NADP(+)-dependent, mitochondrial | 0.307783143 | 2.11.92 ||
MED13 | 2.11 | mediator complex subunit 13 | 0.222155278 | 2.11.88 || METTL6 | 2.11 |
methyltransferase like 6 | 0.254287019 | 2.11.87 || MKLN1 | 2.11 | muskelin 1 | 0.25163679 |
2.11.88 || MLLT3 | 2.11 | myeloid/lymphoid or mixed-lineage leukemia; translocated to, 3 |
0.364863776 | 2.11.93 || MMAA | 2.11 | methylmalonic aciduria (cobalamin deficiency) cblA type |
0.354714539 | 2.11.88 || MMP25 | 2.11 | matrix metallopeptidase 25 | 0.259601703 | 2.11.88 ||
MOCS2 | 2.11 | molybdenum cofactor synthesis 2 | 0.610107611 | 2.11.94 || MORC4 | 2.11 | MORC
family CW-type zinc finger 4 | 1.154152263 | 2.11.92 || MPND | 2.11 | MPN domain containing |
0.254515893 | 2.11.88 || MR1 | 2.11 | major histocompatibility complex, class I-related |
0.267692959 | 2.11.88 || MRPL23 | 2.11 | mitochondrial ribosomal protein L23 | 0.305797447 |
2.11.88 || MRPS7 | 2.11 | mitochondrial ribosomal protein S7 | 0.380177879 | 2.11.88 || MRS2 | 2.11
| MRS2, magnesium transporter | 0.374654346 | 2.11.87 || MSRB3 | 2.11 | methionine sulfoxide
reductase B3 | 0.992582706 | 2.11.95 || MTFP1 | 2.11 | mitochondrial fission process 1 |
0.298762142 | 2.11.87 || MTUS1 | 2.11 | microtubule associated tumor suppressor 1 | 0.566893205 |
- || MUC3A | 2.11 | mucin 3A, cell surface associated | 0.86970088 | 2.11.89 || MYLK | 2.11 |
myosin light chain kinase | 1.49867473 | 2.11.87 || MY010 | 2.11 | myosin X | 0.814300842 |
2.11.95 || MYO1B | 2.11 | myosin IB | 0.476073037 | - || MYO6 | 2.11 | myosin VI | 0.6419066 |
2.11.91 || MYRF | 2.11 | myelin regulatory factor | 0.554442417 | 2.11.89 || NAA16 | 2.11 |
N(alpha)-acetyltransferase 16, NatA auxiliary subunit | 0.423375126 | - || NAALADL1 | 2.11 | N-
acetylated alpha-linked acidic dipeptidase-like 1 | 0.245776186 | 2.11.88 || NACC1 | 2.11 | nucleus
accumbens associated 1 | 0.237676889 | 2.11.89 || NADK2 | 2.11 | NAD kinase 2, mitochondrial |
0.239248157 | 2.11.87 || NCKAP1 | 2.11 | NCK associated protein 1 | 0.271919447 | 2.11.95 ||
NDFIP2 | 2.11 | Nedd4 family interacting protein 2 | 0.4471862 | 2.11.94 || NECAP2 | 2.11 |
NECAP endocytosis associated 2 | 0.218385326 | 2.11.94 || NEDD4 | 2.11 | neural precursor cell
expressed, developmentally down-regulated 4, E3 ubiquitin protein ligase | 0.808755476 | 2.11.91 ||
NEK1 | 2.11 | NIMA related kinase 1 | 0.734884915 | 2.11.88 || NEK6 | 2.11 | NIMA related kinase
6 | 0.448948841 | 2.11.94 || NFKBIE | 2.11 | NFKB inhibitor epsilon | 0.526990652 | 2.11.94 || NLK
| 2.11 | nemo-like kinase | 0.21872192 | 2.11.94 || NLRC4 | 2.11 | NLR family, CARD domain
containing 4 | 0.578897082 | 2.11.87 || NOD2 | 2.11 | nucleotide binding oligomerization domain
containing 2 | 1.301056187 | 2.11.95 || NOL12 | 2.11 | nucleolar protein 12 | 0.284900775 | 2.11.93 ||
NOX4 | 2.11 | NADPH oxidase 4 | 1.108116391 | 2.11.92 || NR2C1 | 2.11 | nuclear receptor
subfamily 2 group C member 1 | 0.454975321 | 2.11.87 || NRG1 | 2.11 | neuregulin 1 | 0.368595639
| 2.11.89 || NT5C3B | 2.11 | 5′-nucleotidase, cytosolic IIIB | 0.244925717 | 2.11.95 || NUCKS1 | 2.11
| nuclear casein kinase and cyclin-dependent kinase substrate 1 | 0.898450913 | 2.11.88 || NUDT16 |
2.11 | nudix hydrolase 16 | 0.326143374 | 2.11.94 || NUDT6 | 2.11 | nudix hydrolase 6 |
0.484537329 | 2.11.87 || NUP133 | 2.11 | nucleoporin 133kDa | 0.592055283 | - || ODC1 | 2.11 |
ornithine decarboxylase 1 | 0.569456629 | - || OLFM1 | 2.11 | olfactomedin 1 | 1.358666772 |
2.11.95 || OLFML1 | 2.11 | olfactomedin like 1 | 0.689591651 | 2.11.95 || OSBPL5 | 2.11 | oxysterol
binding protein like 5 | 0.273643339 | 2.11.89 || OSMR | 2.11 | oncostatin M receptor | 1.503278899
| 2.11.93 || PALD1 | 2.11 | phosphatase domain containing, paladin 1 | 0.555556096 | 2.11.95 ||
PARM1 | 2.11 | prostate androgen-regulated mucin-like protein 1 | 1.023653832 | 2.11.91 || PARVA
| 2.11 | parvin alpha | 0.468790584 | 2.11.95 || PARVG | 2.11 | parvin gamma | 0.537607892 |
2.11.94 || PCDHB14 | 2.11 | protocadherin beta 14 | 1.252426102 | 2.11.93 || PDE10A | 2.11 |
phosphodiesterase 10A | 0.593443067 | 2.11.93 || PDGFA | 2.11 | platelet derived growth factor
subunit A | 0.773068746 | 2.11.94 || PDGFRA | 2.11 | platelet derived growth factor receptor alpha |
0.736213488 | 2.11.90 || PDZD8 | 2.11 | PDZ domain containing 8 | 0.372559184 | 2.11.94 ||
PHACTR2 | 2.11 | phosphatase and actin regulator 2 | 0.37221586 | 2.11.87 || PID1 | 2.11 |
phosphotyrosine interaction domain containing 1 | 0.433982727 | 2.11.96 || PIGX | 2.11 |
phosphatidylinositol glycan anchor biosynthesis class X | 0.294978944 | 2.11.88 || PIK3AP1 | 2.11 |
phosphoinositide-3-kinase adaptor protein 1 | 0.385622164 | 2.11.94 || PLA2G12A | 2.11 |
phospholipase A2 group XIIA | 0.445961327 | 2.11.87 || PLA2R1 | 2.11 | phospholipase A2 receptor
1 | 0.644370683 | 2.11.89 || PLAC9 | 2.11 | placenta specific 9 | 0.857845635 | 2.11.95 || PLCG2 |
2.11 | phospholipase C gamma 2 | 0.220766749 | - || PLEK | 2.11 | pleckstrin | 0.551911738 |
2.11.87 || PLEKHG4 | 2.11 | pleckstrin homology and RhoGEF domain containing G4 |
0.325898829 | 2.11.92 || PLEKHO1 | 2.11 | pleckstrin homology domain containing O1 |
0.614364413 | 2.11.94 || PLS3 | 2.11 | plastin 3 | 0.294103102 | 2.11.91 || PMP22 | 2.11 | peripheral
myelin protein 22 | 0.313324977 | 2.11.92 || PNKD | 2.11 | paroxysmal nonkinesigenic dyskinesia |
0.245392908 | 2.11.95 || PPIC | 2.11 | peptidylprolyl isomerase C | 0.357138142 | 2.11.90 || PPM1H
| 2.11 | protein phosphatase, Mg2+/Mn2+ dependent 1H | 0.613125859 | 2.11.89 || PPP1R37 | 2.11 |
protein phosphatase 1 regulatory subunit 37 | 0.227052078 | 2.11.88 || PRDX1 | 2.11 | peroxiredoxin
1 | 0.316156677 | 2.11.88 || PRELP | 2.11 | proline/arginine-rich end leucine-rich repeat protein |
1.601044294 | 2.11.92 || PRICKLE2 | 2.11 | prickle planar cell polarity protein 2 | 0.782184554 |
2.11.96 || PRKG1 | 2.11 | protein kinase, cGMP-dependent, type I | 0.774538466 | 2.11.91 || PRMT6
| 2.11 | protein arginine methyltransferase 6 | 0.318595128 | 2.11.91 || PRRX2 | 2.11 | paired related
homeobox 2 | 1.039433208 | 2.11.90 || PTBP2 | 2.11 | polypyrimidine tract binding protein 2 |
0.219911518 | 2.11.95 || PTGER3 | 2.11 | prostaglandin E receptor 3 | 1.554612837 | 2.11.89 ||
PTGES2 | 2.11 | prostaglandin E synthase 2 | 0.430878063 | 2.11.88 || PTPN14 | 2.11 | protein
tyrosine phosphatase, non-receptor type 14 | 0.630354757 | 2.11.87 || PTPN7 | 2.11 | protein tyrosine
phosphatase, non-receptor type 7 | 0.261209531 | 2.11.91 || PTPRG | 2.11 | protein tyrosine
phosphatase, receptor type G | 0.965505574 | 2.11.91 || PTRF | 2.11 | polymerase I and transcript
release factor | 0.401843121 | 2.11.95 || PURA | 2.11 | purine-rich element binding protein A |
0.261900751 | 2.11.87 || PUS3 | 2.11 | pseudouridylate synthase 3 | 0.42898479 | 2.11.94 ||
PYCARD | 2.11 | PYD and CARD domain containing | 0.25248098 | 2.11.94 || QSER1 | 2.11 |
glutamine and serine rich 1 | 0.232034763 | 2.11.88 || RAB11FIP2 | 2.11 | RAB11 family interacting
protein 2 (class I) | 0.371953995 | 2.11.88 || RAB23 | 2.11 | RAB23, member RAS oncogene family
| 0.547504018 | 2.11.87 || RAB40B | 2.11 | RAB40B, member RAS oncogene family | 0.691557143
| 2.11.95 || RAI2 | 2.11 | retinoic acid induced 2 | 0.548407973 | 2.11.87 || RALBP1 | 2.11 | ralA
binding protein 1 | 0.422724086 | 2.11.88 || RALGPS2 | 2.11 | Ral GEF with PH domain and SH3
binding motif 2 | 0.646081783 | - || RAMP2 | 2.11 | receptor (G protein-coupled) activity modifying
protein 2 | 0.817071625 | - || RASAL3 | 2.11 | RAS protein activator like 3 | 0.536053481 | - ||
RASGRP2 | 2.11 | RAS guanyl releasing protein 2 | 0.253098453 | 2.11.91 || RASL10B | 2.11 | RAS
like family 10 member B | 0.225353115 | 2.11.88 || RASSF4 | 2.11 | Ras association domain family
member 4 | 0.577137313 | 2.11.95 || RBAK | 2.11 | RB associated KRAB zinc finger | 0.234868336
| 2.11.88 || RBM38 | 2.11 | RNA binding motif protein 38 | 0.649239844 | - || RBM43 | 2.11 | RNA
binding motif protein 43 | 0.800925923 | - || RELL1 | 2.11 | RELT like 1 | 0.293016625 | 2.11.95 ||
RERGL | 2.11 | RERG like | 2.458315954 | 2.11.94 || REV3L | 2.11 | REV3 like, DNA directed
polymerase zeta catalytic subunit | 0.376334466 | 2.11.92 || RFC4 | 2.11 | replication factor C
subunit 4 | 0.345789189 | - || RGMB | 2.11 | repulsive guidance molecule family member b |
0.496642884 | 2.11.95 || RIN1 | 2.11 | Ras and Rab interactor 1 | 0.339814741 | 2.11.89 || RNASE6 |
2.11 | ribonuclease A family member k6 | 0.443789107 | - || RNASET2 | 2.11 | ribonuclease T2 |
0.455239506 | 2.11.94 || RND3 | 2.11 | Rho family GTPase 3 | 0.428249245 | 2.11.95 || RNF144B |
2.11 | ring finger protein 144B | 0.35329375 | 2.11.87 || RNF180 | 2.11 | ring finger protein 180 |
0.349359912 | 2.11.94 || ROBO1 | 2.11 | roundabout guidance receptor 1 | 0.513564066 | 2.11.95 ||
ROR1 | 2.11 | receptor tyrosine kinase-like orphan receptor 1 | 0.546233645 | - || RORA | 2.11 |
RAR related orphan receptor A | 0.401012354 | 2.11.87 || RRAS2 | 2.11 | related RAS viral (r-ras)
oncogene homolog 2 | 2.046008627 | 2.11.94 || RUNX1T1 | 2.11 | runt related transcription factor 1;
translocated to, 1 (cyclin D related) | 1.548689058 | 2.11.87 || SACS | 2.11 | sacsin molecular
chaperone | 0.485582191 | 2.11.93 || SAMD4A | 2.11 | sterile alpha motif domain containing 4A |
0.447671749 | 2.11.87 || SAP30 | 2.11 | Sin3 A associated protein 30kDa | 0.605909372 | 2.11.94 ||
SASH1 | 2.11 | SAM and SH3 domain containing 1 | 0.619338966 | 2.11.91 || SCNNIA | 2.11 |
sodium channel epithelial 1 alpha subunit | 0.337628112 | 2.11.89 || SCO2 | 2.11 | SCO2
cytochrome c oxidase assembly protein | 0.762355096 | 2.11.94 || SDF2L1 | 2.11 | stromal cell
derived factor 2 like 1 | 0.349428243 | 2.11.88 || SEC16B | 2.11 | SEC16 homolog B, endoplasmic
reticulum export factor | 0.340832577 | 2.11.91 || SELP | 2.11 | selectin P | 1.790362768 | 2.11.91 ||
SEMA4A | 2.11 | semaphorin 4A | 0.365692765 | 2.11.87 || SEMA7A | 2.11 | semaphorin 7A (John
Milton Hagen blood group) | 0.302560808 | 2.11.88 || SEPT8 | 2.11 | septin 8 | 0.267651618 |
2.11.93 || SERPINB1 | 2.11 | serpin peptidase inhibitor, clade B (ovalbumin), member 1 |
0.252267199 | 2.11.95 || SFN | 2.11 | stratifin | 0.467860274 | 2.11.94 || SFRP1 | 2.11 | secreted
frizzled-related protein 1 | 2.692419347 | 2.11.94 || SFSWAP | 2.11 | splicing factor, suppressor of
white-apricot homolog | 0.375126962 | 2.11.88 || SH3PXD2A | 2.11 | SH3 and PX domains 2A |
0.386457248 | 2.11.93 || SHOX2 | 2.11 | short stature homeobox 2 | 1.268819877 | 2.11.87 ||
SIGLEC10 | 2.11 | sialic acid binding Ig like lectin 10 | 1.113832539 | 2.11.87 || SIGLEC9 | 2.11 |
sialic acid binding Ig like lectin 9 | 0.430433642 | 2.11.96 || SIKE1 | 2.11 | suppressor of IKBKE 1 |
0.386816426 | - || SIVA1 | 2.11 | SIVA1 apoptosis inducing factor | 0.295265925 | 2.11.88 || SKA2 |
2.11 | spindle and kinetochore associated complex subunit 2 | 0.243261777 | 2.11.95 || SLA | 2.11 |
Src-like-adaptor | 0.247621608 | 2.11.94 || SLAMF8 | 2.11 | SLAM family member 8 | 5.562850311
| 2.11.94 || SLC16A14 | 2.11 | solute carrier family 16 member 14 | 0.51291194 | 2.11.95 ||
SLC16A6 | 2.11 | solute carrier family 16 member 6 | 1.372102975 | 2.11.93 || SLC17A9 | 2.11 |
solute carrier family 17 member 9 | 1.29964194 | 2.11.87 || SLC22A7 | 2.11 | solute carrier family
22 member 7 | 0.320622105 | 2.11.87 || SLC2A10 | 2.11 | solute carrier family 2 member 10 |
1.284795292 | 2.11.90 || SLC2A5 | 2.11 | solute carrier family 2 member 5 | 0.855621559 | 2.11.96 ||
SLC5A3 | 2.11 | solute carrier family 5 member 3 | 1.125767521 | 2.11.93 || SLCO2A1 | 2.11 |
solute carrier organic anion transporter family member 2A1 | 0.595840495 | 2.11.93 || SLIT2 | 2.11 |
slit guidance ligand 2 | 1.534824777 | 2.11.93 || SLPI | 2.11 | secretory leukocyte peptidase inhibitor
| 0.985296136 | 2.11.94 || SMAP2 | 2.11 | small ArfGAP2 | 0.587418747 | 2.11.94 || SMARCA1 |
2.11 | SWI/SNF related, matrix associated, actin dependent regulator of chromatin, subfamily a,
member 1 | 0.518225364 | 2.11.91 || SMCO4 | 2.11 | single-pass membrane protein with coiled-coil
domains 4 | 0.567333671 | 2.11.94 || SNRNP48 | 2.11 | small nuclear ribonucleoprotein U11/U12
subunit 48 | 0.385402187 | 2.11.88 || SNX21 | 2.11 | sorting nexin family member 21 | 1.248192959
| 2.11.87 || SOBP | 2.11 | sine oculis binding protein homolog | 1.928931461 | 2.11.87 || SPA17 |
2.11 | sperm autoantigenic protein 17 | 0.285280326 | 2.11.89 || SPAG16 | 2.11 | sperm associated
antigen 16 | 0.915477114 | 2.11.87 || SPATA7 | 2.11 | spermatogenesis associated 7 | 1.368215056 |
2.11.87 || SPIN4 | 2.11 | spindlin family member 4 | 0.770418964 | - || SPOCK1 | 2.11 |
sparc/osteonectin, cwcv and kazal-like domains proteoglycan (testican) 1 | 1.725208877 | 2.11.91 ||
SRGAP2C | 2.11 | SLIT-ROBO Rho GTPase activating protein 2C | 0.480654427 | 2.11.88 ||
SSC4D | 2.11 | scavenger receptor cysteine rich family, 4 domains | 0.291927929 | 2.11.88 ||
STARD13 | 2.11 | StAR related lipid transfer domain containing 13 | 0.294783875 | 2.11.95 ||
SYBU | 2.11 | syntabulin | 0.87407809 | 2.11.92 || SYNGR2 | 2.11 | synaptogyrin 2 | 0.571686213 |
2.11.94 || SYNPO | 2.11 | synaptopodin | 0.295647298 | 2.11.87 || SYTL2 | 2.11 | synaptotagmin like
2 | 1.265626995 | 2.11.87 || TAPBPL | 2.11 | TAP binding protein like | 1.128158128 | 2.11.94 ||
TBC1D12 | 2.11 | TBC1 domain family member 12 | 0.22746469 | 2.11.92 || TBC1D16 | 2.11 |
TBC1 domain family member 16 | 0.617692994 | 2.11.92 || TBC1D8B | 2.11 | TBC1 domain family
member 8B | 0.93406443 | 2.11.93 || TBLIX | 2.11 | transducin (beta)-like 1X-linked | 0.489219089
| 2.11.88 || TBL3 | 2.11 | transducin (beta)-like 3 | 0.279280529 | 2.11.88 || TCEAL8 | 2.11 |
transcription elongation factor A like 8 | 0.244030796 | 2.11.90 || TCEAL9 | 2.11 | transcription
elongation factor A like 9 | 0.322005675 | 2.11.90 || TCF4 | 2.11 | transcription factor 4 |
0.458828535 | 2.11.87 || TFDP2 | 2.11 | transcription factor Dp-2 (E2F dimerization partner 2) |
0.736516249 | 2.11.91 || TFPI | 2.11 | tissue factor pathway inhibitor | 0.566235882 | 2.11.87 ||
TGFB1I1 | 2.11 | transforming growth factor beta 1 induced transcript 1 | 0.507765297 | - ||
TGFBR3 | 2.11 | transforming growth factor beta receptor III | 1.507257805 | 2.11.92 || THAP3 |
2.11 | THAP domain containing, apoptosis associated protein 3 | 0.568537294 | 2.11.87 || THAP5 |
2.11 | THAP domain containing 5 | 0.263448751 | - || TIMM44 | 2.11 | translocase of inner
mitochondrial membrane 44 | 0.255360129 | 2.11.88 || TLR2 | 2.11 | toll like receptor 2 |
1.299433362 | 2.11.94 || TMA16 | 2.11 | translation machinery associated 16 homolog |
0.604362157 | 2.11.88 || TMEFF2 | 2.11 | transmembrane protein with EGF like and two follistatin
like domains 2 | 0.709341931 | 2.11.91 || TMEM108 | 2.11 | transmembrane protein 108 |
2.284817953 | 2.11.87 || TMEM136 | 2.11 | transmembrane protein 136 | 0.50399416 | 2.11.93 ||
TMEM163 | 2.11 | transmembrane protein 163 | 0.269732545 | 2.11.87 || TMEM192 | 2.11 |
transmembrane protein 192 | 0.550107757 | 2.11.88 || TMEM208 | 2.11 | transmembrane protein
208 | 0.483224276 | 2.11.88 || TMEM237 | 2.11 | transmembrane protein 237 | 0.280687141 |
2.11.87 || TMEM51 | 2.11 | transmembrane protein 51 | 0.770185115 | 2.11.95 || TMOD2 | 2.11 |
tropomodulin 2 | 0.309966241 | - || TMTC3 | 2.11 | transmembrane and tetratricopeptide repeat
containing 3 | 0.501999476 | - || TNFAIP8L3 | 2.11 | TNF alpha induced protein 8 like 3 |
0.256780873 | 2.11.89 || TNFRSF11A | 2.11 | tumor necrosis factor receptor superfamily member
11a | 1.490932349 | 2.11.95 || TNFRSF21 | 2.11 | tumor necrosis factor receptor superfamily
member 21 | 0.542314989 | 2.11.96 || TNS2 | 2.11 | tensin 2 | 0.425631795 | 2.11.92 || TRERF1 |
2.11 | transcriptional regulating factor 1 | 0.700646116 | 2.11.93 || TRIB1 | 2.11 | tribbles
pseudokinase 1 | 0.406969487 | 2.11.93 || TRIM13 | 2.11 | tripartite motif containing 13 |
0.67103634 | 2.11.93 || TRIM56 | 2.11 | tripartite motif containing 56 | 0.83888073 | - || TRIP11 |
2.11 | thyroid hormone receptor interactor 11 | 0.306304647 | 2.11.87 || TRNT1 | 2.11 | tRNA
nucleotidyl transferase, CCA-adding, 1 | 0.442827291 | 2.11.93 || TRPV2 | 2.11 | transient receptor
potential cation channel subfamily V member 2 | 0.317197105 | 2.11.88 || TSC22D1 | 2.11 | TSC22
domain family member 1 | 0.371148126 | 2.11.95 || TSPAN15 | 2.11 | tetraspanin 15 | 0.512575205 |
2.11.93 || TTC25 | 2.11 | tetratricopeptide repeat domain 25 | 0.219233441 | 2.11.87 || TTC28 | 2.11 |
tetratricopeptide repeat domain 28 | 1.083496716 | 2.11.93 || TTC37 | 2.11 | tetratricopeptide repeat
domain 37 | 0.304527669 | - || TTC6 | 2.11 | tetratricopeptide repeat domain 6 | 0.666114507 |
2.11.94 || TTC8 | 2.11 | tetratricopeptide repeat domain 8 | 0.856140725 | 2.11.93 || TTLL9 | 2.11 |
tubulin tyrosine ligase like 9 | 0.393028309 | 2.11.89 || TUBA4A | 2.11 | tubulin alpha 4a |
0.631849282 | - || TWIST1 | 2.11 | twist family bHLH transcription factor 1 | 0.835937996 | 2.11.95
|| TWISTNB | 2.11 | TWIST neighbor | 3.257124484 | 2.11.90 || TXNDC11 | 2.11 | thioredoxin
domain containing 11 | 0.579050214 | 2.11.88 || TYMP | 2.11 | thymidine phosphorylase |
0.947070876 | 2.11.94 || UBN1 | 2.11 | ubinuclein 1 | 0.333970619 | 2.11.88 || UBTD2 | 2.11 |
ubiquitin domain containing 2 | 0.353079528 | 2.11.87 || UCP2 | 2.11 | uncoupling protein 2
(mitochondrial, proton carrier) | 0.322404283 | 2.11.90 || UNKL | 2.11 | unkempt family like zinc
finger | 0.228147833 | - || VCAN | 2.11 | versican | 0.446645843 | - || VGLL3 | 2.11 | vestigial like
family member 3 | 1.251616228 | 2.11.91 || VMO1 | 2.11 | vitelline membrane outer layer 1
homolog (chicken) | 0.340727067 | - || WARS2 | 2.11 | tryptophanyl tRNA synthetase 2,
mitochondrial | 0.70446101 | 2.11.96 || WLS | 2.11 | wntless Wnt ligand secretion mediator |
1.31650603 | 2.11.96 || WNT5A | 2.11 | wingless-type MMTV integration site family member 5A |
0.412515726 | 2.11.94 || WRN | 2.11 | Werner syndrome RecQ like helicase | 0.247501696 | 2.11.89
|| YAP1 | 2.11 | Yes associated protein 1 | 0.605460411 | 2.11.95 || YES1 | 2.11 | YES proto-
oncogene 1, Src family tyrosine kinase | 0.625134871 | 2.11.87 || ZBED3 | 2.11 | zinc finger BED-
type containing 3 | 0.289258338 | 2.11.87 || ZBED6 | 2.11 | zinc finger BED-type containing 6 |
0.965378396 | 2.11.87 || ZBTB37 | 2.11 | zinc finger and BTB domain containing 37 | 0.423861555 |
-| | ZBTB44 | 2.11 | zinc finger and BTB domain containing 44 | 0.66153182 | 2.11.88 || ZBTB8A |
2.11 | zinc finger and BTB domain containing 8A | 0.843068611 | 2.11.87 || ZC3H14 | 2.11 | zinc
finger CCCH-type containing 14 | 0.431930295 | 2.11.88 || ZCCHC24 | 2.11 | zinc finger CCHC-
type containing 24 | 0.958574416 | 2.11.87 || ZDHHC9 | 2.11 | zinc finger DHHC-type containing 9
| 0.360693978 | 2.11.94 || ZEB1 | 2.11 | zinc finger E-box binding homeobox 1 | 0.734907942 |
2.11.91 || ZFAS1 | 2.11 | ZNFX1 antisense RNA 1 | 0.216926924 | 2.11.88 || ZFHX4 | 2.11 | zinc
finger homeobox 4 | 1.487261107 | 2.11.96 || ZFP1 | 2.11 | ZFP1 zinc finger protein | 0.656534532 |
2.11.94 || ZFP90 | 2.11 | ZFP90 zinc finger protein | 1.647601439 | 2.11.88 || ZHX1 | 2.11 | zinc
fingers and homeoboxes 1 | 0.399846336 | 2.11.88 || ZMYM3 | 2.11 | zinc finger MYM-type
containing 3 | 0.228678583 | 2.11.89 || ZNF112 | 2.11 | zinc finger protein 112 | 0.263784849 |
2.11.87 || ZNF213 | 2.11 | zinc finger protein 213 | 0.279066572 | 2.11.95 || ZNF264 | 2.11 | zinc
finger protein 264 | 0.659488215 | - || ZNF268 | 2.11 | zinc finger protein 268 | 0.89780186 | 2.11.93
|| ZNF271P | 2.11 | zinc finger protein 271, pseudogene | 0.750163201 | 2.11.87 || ZNF329 | 2.11 |
zinc finger protein 329 | 0.232425809 | 2.11.93 || ZNF426 | 2.11 | zinc finger protein 426 |
1.54676683 | 2.11.88 || ZNF432 | 2.11 | zinc finger protein 432 | 0.249140608 | - || ZNF443 | 2.11 |
zinc finger protein 443 | 0.241857926 | 2.11.88 || ZNF45 | 2.11 | zinc finger protein 45 |
0.255425089 | 2.11.91 || ZNF462 | 2.11 | zinc finger protein 462 | 1.060098814 | 2.11.94 || ZNF503 |
2.11 | zinc finger protein 503 | 0.935714707 | 2.11.93 || ZNF507 | 2.11 | zinc finger protein 507 |
0.334228087 | 2.11.96 || ZNF512B | 2.11 | zinc finger protein 512B | 0.500698568 | 2.11.93 ||
ZNF567 | 2.11 | zinc finger protein 567 | 1.122144387 | 2.11.87 || ZNF600 | 2.11 | zinc finger
protein 600 | 0.255226768 | 2.11.91 || ZNF608 | 2.11 | zinc finger protein 608 | 1.201445355 |
2.11.91 || ZNF618 | 2.11 | zinc finger protein 618 | 1.07071906 | 2.11.93 || ZNF703 | 2.11 | zinc
finger protein 703 | 0.549888949 | 2.11.94 || ZNF823 | 2.11 | zinc finger protein 823 | 0.508288076 |
2.11.87 || ZNF827 | 2.11 | zinc finger protein 827 | 0.939881938 | 2.11.91 || ZNF83 | 2.11 | zinc
finger protein 83 | 0.311507378 | 2.11.87 || ACADSB | 2.11.87 | acyl-CoA dehydrogenase,
short/branched chain | 0.448305681 | 2.11.87 || ACOX2 | 2.11.87 | acyl-CoA oxidase 2, branched
chain | 0.589616328 | 2.11.87 || ADD3 | 2.11.87 | adducin 3 | 0.607985917 | 2.11.87 || AMIGO2 |
2.11.87 | adhesion molecule with Ig-like domain 2 | 0.814772914 | 2.11.87 || BMPRIA | 2.11.87 |
bone morphogenetic protein receptor type 1A | 0.613503708 | 2.11.87 || C7orf55 | 2.11.87 |
chromosome 7 open reading frame 55 | 0.668742837 | 2.11.87 || CDC37L1 | 2.11.87 | cell division
cycle 37-like 1 | 0.231469743 | 2.11.87 || CEP290 | 2.11.87 || centrosomal protein 290kDa |
0.263768045 | 2.11.87 || CLOCK | 2.11.87 | clock circadian regulator | 0.293393216 | 2.11.87 ||
COL27A1 | 2.11.87 | collagen type XXVII alpha 1 | 0.892869302 | 2.11.87 || CWC27 | 2.11.87 |
CWC27 spliceosome associated protein homolog | 0.243405515 | 2.11.87 || DAAM1 | 2.11.87 |
dishevelled associated activator of morphogenesis 1 | 0.471280572 | 2.11.87 || DCLK2 | 2.11.87 |
doublecortin like kinase 2 | 0.511160655 | 2.11.87 || DDR2 | 2.11.87 || discoidin domain receptor
tyrosine kinase 2 | 0.942052657 | 2.11.87 || DIXDC1 | 2.11.87 | DIX domain containing 1 |
0.759440001 | 2.11.87 || DYNC2LI1 | 2.11.87 | dynein cytoplasmic 2 light intermediate chain 1 |
0.626974122 | 2.11.87 || EBF1 | 2.11.87 | early B-cell factor 1 | 0.937984168 | 2.11.87 || EBF2 |
2.11.87 | early B-cell factor 2 | 3.351821547 | 2.11.87 | EFS | 2.11.87 | embryonal Fyn-associated
substrate | 0.591158755 | 2.11.87 || EYA4 | 2.11.87 | EYA transcriptional coactivator and
phosphatase 4 | 0.759943865 | 2.11.87 || FAM210B | 2.11.87 | family with sequence similarity 210
member B | 0.277177641 | 2.11.87 || FBXL17 | 2.11.87 | F-box and leucine-rich repeat protein 17 |
0.321221777 | 2.11.87 || FGF13 | 2.11.87 | fibroblast growth factor 13 | 0.987877209 | 2.11.87 ||
FNDC4 | 2.11.87 | fibronectin type III domain containing 4 | 0.4953917 | 2.11.87 || FYN | 2.11.87 |
FYN proto-oncogene, Src family tyrosine kinase | 0.285535416 | 2.11.87 || GLPIR | 2.11.87 |
glucagon like peptide 1 receptor | 0.286795346 | 2.11.87 || GPHN | 2.11.87 | gephyrin | 0.379120059
| 2.11.87 || GPR65 | 2.11.87 | G protein-coupled receptor 65 | 0.526628376 | 2.11.87 || HES1 |
2.11.87 | hes family bHLH transcription factor 1 | 0.693985311 | 2.11.87 || IBSP | 2.11.87 | integrin
binding sialoprotein | 0.255171768 | 2.11.87 || JPX | 2.11.87 | JPX transcript, XIST activator (non-
protein coding) | 0.780111483 | 2.11.87 || KCNT2 | 2.11.87 | potassium sodium-activated channel
subfamily T member 2 | 1.179764931 | 2.11.87 || KCTD3 | 2.11.87 | potassium channel
tetramerization domain containing 3 | 0.745709664 | 2.11.87 || KMT5A | 2.11.87 | lysine
methyltransferase 5A | 0.451503802 | 2.11.87 || LEPROTL1 | 2.11.87 | leptin receptor overlapping
transcript-like 1 | 0.369399864 | 2.11.87 || LIMCH1 | 2.11.87 | LIM and calponin homology
domains 1 | 1.258866277 | 2.11.87 || LOC101927027 | 2.11.87 | uncharacterized LOC101927027 |
0.275757716 | 2.11.87 || LYRM5 | 2.11.87 | LYR motif containing 5 | 0.276852304 | 2.11.87 || LYZ
| 2.11.87 | lysozyme | 0.298216054 | 2.11.87 || MAP9 | 2.11.87 | microtubule associated protein 9 |
1.228570929 | 2.11.87 || MATK | 2.11.87 | megakaryocyte-associated tyrosine kinase | 1.377160924
| 2.11.87 || METTL6 | 2.11.87 | methyltransferase like 6 | 0.254287019 | 2.11.87 || MRS2 | 2.11.87 |
MRS2, magnesium transporter | 0.374654346 | 2.11.87 || MTFP1 | 2.11.87 | mitochondrial fission
process 1 | 0.298762142 | 2.11.87 || MYLK | 2.11.87 | myosin light chain kinase | 1.49867473 |
2.11.87 || NADK2 | 2.11.87 | NAD kinase 2, mitochondrial | 0.239248157 | 2.11.87 || NLRC4 |
2.11.87 | NLR family, CARD domain containing 4 | 0.578897082 | 2.11.87 || NR2C1 | 2.11.87 |
nuclear receptor subfamily 2 group C member 1 | 0.454975321 | 2.11.87 || NUDT6 | 2.11.87 | nudix
hydrolase 6 | 0.484537329 | 2.11.87 || PHACTR2 | 2.11.87 | phosphatase and actin regulator 2 |
0.37221586 | 2.11.87 || PLA2G12A | 2.11.87 | phospholipase A2 group XIIA | 0.445961327 |
2.11.87 || PLEK | 2.11.87 | pleckstrin | 0.551911738 | 2.11.87 || PTPN14 | 2.11.87 | protein tyrosine
phosphatase, non-receptor type 14 | 0.630354757 | 2.11.87 || PURA | 2.11.87 | purine-rich element
binding protein A | 0.261900751 | 2.11.87 || RAB23 | 2.11.87 | RAB23, member RAS oncogene
family | 0.547504018 | 2.11.87 || RAI2 | 2.11.87 | retinoic acid induced 2 | 0.548407973 | 2.11.87 ||
RNF144B | 2.11.87 | ring finger protein 144B | 0.35329375 | 2.11.87 || RORA | 2.11.87 | RAR
related orphan receptor A | 0.401012354 | 2.11.87 || RUNX1T1 | 2.11.87 | runt related transcription
factor 1; translocated to, 1 (cyclin D related) | 1.548689058 | 2.11.87 || SAMD4A | 2.11.87 | sterile
alpha motif domain containing 4A | 0.447671749 | 2.11.87 || SEMA4A | 2.11.87 | semaphorin 4A |
0.365692765 | 2.11.87 || SHOX2 | 2.11.87 | short stature homeobox 2 | 1.268819877 | 2.11.87 ||
SIGLEC10 | 2.11.87 | sialic acid binding Ig like lectin 10 | 1.113832539 | 2.11.87 || SLC17A9 |
2.11.87 | solute carrier family 17 member 9 | 1.29964194 | 2.11.87 || SLC22A7 | 2.11.87 | solute
carrier family 22 member 7 | 0.320622105 | 2.11.87 || SNX21 | 2.11.87 | sorting nexin family
member 21 | 1.248192959 | 2.11.87 || SOBP | 2.11.87 | sine oculis binding protein homolog |
1.928931461 | 2.11.87 || SPAG16 | 2.11.87 | sperm associated antigen 16 | 0.915477114 | 2.11.87 ||
SPATA7 | 2.11.87 | spermatogenesis associated 7 | 1.368215056 | 2.11.87 || SYNPO | 2.11.87 |
synaptopodin | 0.295647298 | 2.11.87 || SYTL2 | 2.11.87 | synaptotagmin like 2 | 1.265626995 |
2.11.87 || TCF4 | 2.11.87 | transcription factor 4 | 0.458828535 | 2.11.87 || TFPI | 2.11.87 | tissue
factor pathway inhibitor | 0.566235882 | 2.11.87 || THAP3 | 2.11.87 | THAP domain containing,
apoptosis associated protein 3 | 0.568537294 | 2.11.87 || TMEM108 | 2.11.87 | transmembrane
protein 108 | 2.284817953 | 2.11.87 || TMEM163 | 2.11.87 | transmembrane protein 163 |
0.269732545 | 2.11.87 || TMEM237 | 2.11.87 | transmembrane protein 237 | 0.280687141 | 2.11.87 ||
TRIP11 | 2.11.87 | thyroid hormone receptor interactor 11 | 0.306304647 | 2.11.87 || TTC25 |
2.11.87 | tetratricopeptide repeat domain 25 | 0.219233441 | 2.11.87 || UBTD2 | 2.11.87 | ubiquitin
domain containing 2 | 0.353079528 | 2.11.87 || YES1 | 2.11.87 | YES proto-oncogene 1, Src family
tyrosine kinase | 0.625134871 | 2.11.87 || ZBED3 | 2.11.87 | zinc finger BED-type containing 3 |
0.289258338 | 2.11.87 || ZBED6 | 2.11.87 | zinc finger BED-type containing 6 | 0.965378396 |
2.11.87 || ZBTB8A | 2.11.87 | zinc finger and BTB domain containing 8A | 0.843068611 | 2.11.87 ||
ZCCHC24 | 2.11.87 | zinc finger CCHC-type containing 24 | 0.958574416 | 2.11.87 || ZNF112 |
2.11.87 | zinc finger protein 112 | 0.263784849 | 2.11.87 || ZNF271P | 2.11.87 | zinc finger protein
271, pseudogene | 0.750163201 | 2.11.87 || ZNF567 | 2.11.87 | zinc finger protein 567 | 1.122144387
| 2.11.87 || ZNF823 | 2.11.87 | zinc finger protein 823 | 0.508288076 | 2.11.87 || ZNF83 | 2.11.87 |
zinc finger protein 83 | 0.311507378 | 2.11.87 || ABCD3 | 2.11.88 | ATP binding cassette subfamily
D member 3 | 0.303894131 | 2.11.88 || ACOT7 | 2.11.88 | acyl-CoA thioesterase 7 | 1.191420448 |
2.11.88 || AKRIA1 | 2.11.88 | aldo-keto reductase family 1, member Al (aldehyde reductase) |
0.266734234 | 2.11.88 || ANKRD36B | 2.11.88 | ankyrin repeat domain 36B | 0.705201592 | 2.11.88
|| BAX | 2.11.88 | BCL2-associated X protein | 0.286362311 | 2.11.88 || C19orf68 | 2.11.88 |
chromosome 19 open reading frame 68 | 0.454827829 | 2.11.88 || CASP6 | 2.11.88 | caspase 6 |
0.798729681 | 2.11.88 || CBR1 | 2.11.88 | carbonyl reductase 1 | 0.34570911 | 2.11.88 || CCAR1 |
2.11.88 | cell division cycle and apoptosis regulator 1 | 0.31308275 | 2.11.88 || CD38 | 2.11.88 |
CD38 molecule | 2.150406121 | 2.11.88 || CEBPA-AS1 | 2.11.88 | CEBPA antisense RNA 1 (head
to head) | 0.219803491 | 2.11.88 || CEMP1 | 2.11.88 | cementum protein 1 | 0.327109293 | 2.11.88 ||
CEP126 | 2.11.88 || centrosomal protein 126kDa | 1.892868245 | 2.11.88 || CEP164 | 2.11.88 |
centrosomal protein 164kDa | 0.955611752 | 2.11.88 || COL7A1 | 2.11.88 | collagen type VII alpha
1 | 0.368213732 | 2.11.88 || DDX54 | 2.11.88 | DEAD-box helicase 54 | 0.613719873 | 2.11.88 ||
DHX33 | 2.11.88 | DEAH-box helicase 33 | 0.378118657 | 2.11.88 || DNAJB9 | 2.11.88 | DnaJ heat
shock protein family (Hsp40) member B9 | 0.375758976 | 2.11.88 || DPP4 | 2.11.88 | dipeptidyl
peptidase 4 | 1.0578736 | 2.11.88 || EDEM2 | 2.11.88 | ER degradation enhancer, mannosidase
alpha-like 2 | 0.286374549 | 2.11.88 || FIGN | 2.11.88 | fidgetin | 1.045218876 | 2.11.88 || GTPBP2 |
2.11.88 | GTP binding protein 2 | 1.042769987 | 2.11.88 || INSR | 2.11.88 | insulin receptor |
1.130743908 | 2.11.88 || INTS2 | 2.11.88 | integrator complex subunit 2 | 0.310307903 | 2.11.88 ||
IRAK1BP1 | 2.11.88 | interleukin 1 receptor associated kinase 1 binding protein 1 | 0.384567859 |
2.11.88 || ISYNA1 | 2.11.88 | inositol-3-phosphate synthase 1 | 0.467806734 | 2.11.88 || ITPR2 |
2.11.88 | inositol 1,4,5-trisphosphate receptor type 2 | 0.577070542 | 2.11.88 || IVNS1ABP | 2.11.88
| influenza virus NS1A binding protein | 0.302793942 | 2.11.88 || KLHL20 | 2.11.88 | kelch like
family member 20 | 0.472326564 | 2.11.88 || KLHL8 | 2.11.88 | kelch like family member 8 |
0.377497362 | 2.11.88 || KLK15 | 2.11.88 | kallikrein related peptidase 15 | 0.583177926 | 2.11.88 ||
KPNA5 | 2.11.88 | karyopherin subunit alpha 5 | 0.471170469 | 2.11.88 || LAMP2 | 2.11.88 |
lysosomal associated membrane protein 2 | 0.317829107 | 2.11.88 || LAMTOR4 | 2.11.88 | late
endosomal/lysosomal adaptor, MAPK and MTOR activator 4 | 0.21450644 | 2.11.88 || LOC146880 |
2.11.88 | Rho GTPase activating protein 27 pseudogene | 0.51948776 | 2.11.88 || LYRM7 | 2.11.88 |
LYR motif containing 7 | 0.547635505 | 2.11.88 || MED13 | 2.11.88 | mediator complex subunit 13 |
0.222155278 | 2.11.88 || MKLN1 | 2.11.88 | muskelin 1 | 0.25163679 | 2.11.88 || MMAA | 2.11.88 |
methylmalonic aciduria (cobalamin deficiency) cblA type | 0.354714539 | 2.11.88 || MMP25 |
2.11.88 | matrix metallopeptidase 25 | 0.259601703 | 2.11.88 || MPND | 2.11.88 | MPN domain
containing | 0.254515893 | 2.11.88 || MR1 | 2.11.88 | major histocompatibility complex, class I-
related | 0.267692959 | 2.11.88 || MRPL23 | 2.11.88 | mitochondrial ribosomal protein L23 |
0.305797447 | 2.11.88 || MRPS7 | 2.11.88 | mitochondrial ribosomal protein S7 | 0.380177879 |
2.11.88 || NAALADL1 | 2.11.88 | N-acetylated alpha-linked acidic dipeptidase-like 1 | 0.245776186
| 2.11.88 || NEK1 | 2.11.88 | NIMA related kinase 1 | 0.734884915 | 2.11.88 || NUCKS1 | 2.11.88 |
nuclear casein kinase and cyclin-dependent kinase substrate 1 | 0.898450913 | 2.11.88 || PIGX |
2.11.88 | phosphatidylinositol glycan anchor biosynthesis class X | 0.294978944 | 2.11.88 ||
PPP1R37 | 2.11.88 | protein phosphatase 1 regulatory subunit 37 | 0.227052078 | 2.11.88 || PRDX1 |
2.11.88 | peroxiredoxin 1 | 0.316156677 | 2.11.88 || PTGES2 | 2.11.88 | prostaglandin E synthase 2 |
0.430878063 | 2.11.88 || QSER1 | 2.11.88 | glutamine and serine rich 1 | 0.232034763 | 2.11.88 ||
RAB11FIP2 | 2.11.88 | RAB11 family interacting protein 2 (class I) | 0.371953995 | 2.11.88 ||
RALBP1 | 2.11.88 | ralA binding protein 1 | 0.422724086 | 2.11.88 || RASL10B | 2.11.88 | RAS like
family 10 member B | 0.225353115 | 2.11.88 || RBAK | 2.11.88 | RB associated KRAB zinc finger |
0.234868336 | 2.11.88 || SDF2L1 | 2.11.88 | stromal cell derived factor 2 like 1 | 0.349428243 |
2.11.88 || SEMA7A | 2.11.88 | semaphorin 7A (John Milton Hagen blood group) | 0.302560808 |
2.11.88 || SFSWAP | 2.11.88 | splicing factor, suppressor of white-apricot homolog | 0.375126962 |
2.11.88 || SIVA1 | 2.11.88 | SIVA1 apoptosis inducing factor | 0.295265925 | 2.11.88 || SNRNP48 |
2.11.88 | small nuclear ribonucleoprotein U11/U12 subunit 48 | 0.385402187 | 2.11.88 || SRGAP2C
| 2.11.88 | SLIT-ROBO Rho GTPase activating protein 2C | 0.480654427 | 2.11.88 || SSC4D |
2.11.88 | scavenger receptor cysteine rich family, 4 domains | 0.291927929 | 2.11.88 || TBL1X |
2.11.88 | transducin (beta)-like 1X-linked | 0.489219089 | 2.11.88 || TBL3 | 2.11.88 | transducin
(beta)-like 3 | 0.279280529 | 2.11.88 || TIMM44 | 2.11.88 | translocase of inner mitochondrial
membrane 44 | 0.255360129 | 2.11.88 || TMA16 | 2.11.88 | translation machinery associated 16
homolog | 0.604362157 | 2.11.88 || TMEM192 | 2.11.88 | transmembrane protein 192 | 0.550107757
| 2.11.88 || TMEM208 | 2.11.88 | transmembrane protein 208 | 0.483224276 | 2.11.88 || TRPV2 |
2.11.88 | transient receptor potential cation channel subfamily V member 2 | 0.317197105 | 2.11.88
|| TXNDC11 | 2.11.88 || thioredoxin domain containing 11 | 0.579050214 | 2.11.88 || UBN1 | 2.11.88
|ubinuclein 1 | 0.333970619 | 2.11.88 || ZBTB44 | 2.11.88 | zinc finger and BTB domain containing
44 | 0.66153182 | 2.11.88 || ZC3H14 | 2.11.88 | zinc finger CCCH-type containing 14 | 0.431930295
| 2.11.88 || ZFAS1 | 2.11.88 | ZNFX1 antisense RNA 1 | 0.216926924 | 2.11.88 || ZFP90 | 2.11.88 |
ZFP90 zinc finger protein | 1.647601439 | 2.11.88 || ZHX1 | 2.11.88 | zinc fingers and homeoboxes
1 | 0.399846336 | 2.11.88 || ZNF426 | 2.11.88 | zinc finger protein 426 | 1.54676683 | 2.11.88 ||
ZNF443 | 2.11.88 | zinc finger protein 443 | 0.241857926 | 2.11.88 || ABAT | 2.11.91 || 4-
aminobutyrate aminotransferase | 0.459696688 | 2.11.91 || ADAMTS5 | 2.11.91 | ADAM
metallopeptidase with thrombospondin type 1 motif 5 | 0.787279475 | 2.11.91 || ARHGEF12 |
2.11.91 | Rho guanine nucleotide exchange factor 12 | 0.253338823 | 2.11.91 || BNC2 | 2.11.91 |
basonuclin 2 | 0.902943933 | 2.11.91 || C22orf39 | 2.11.91 | chromosome 22 open reading frame 39 |
0.215432391 | 2.11.91 || CALD1 | 2.11.91 | caldesmon 1 | 0.418718877 | 2.11.91 || CD248 | 2.11.91 |
CD248 molecule | 0.68823608 | 2.11.91 || CEP57 | 2.11.91 | centrosomal protein 57kDa |
0.366056851 | 2.11.91 || DIAPH2 | 2.11.91 | diaphanous related formin 2 | 0.275314812 | 2.11.91 ||
DIP2C | 2.11.91 | disco interacting protein 2 homolog C | 0.519361999 | 2.11.91 || EBPL | 2.11.91 |
emopamil binding protein like | 0.556331861 | 2.11.91 || FAM92A1 | 2.11.91 | family with sequence
similarity 92 member Al | 0.634098775 | 2.11.91 || GNA11 | 2.11.91 | G protein subunit alpha 11 |
0.295970179 | 2.11.91 || GNG12 | 2.11.91 | G protein subunit gamma 12 | 0.309818361 | 2.11.91 ||
GPSM2 | 2.11.91 || G-protein signaling modulator 2 | 0.564926901 | 2.11.91 || GSTT1 | 2.11.91 |
glutathione S-transferase theta 1 | 1.747692966 | 2.11.91 || HAMP | 2.11.91 | hepcidin antimicrobial
peptide | 0.284354134 | 2.11.91 || IGDCC4 | 2.11.91 | immunoglobulin superfamily, DCC subclass,
member 4 | 1.34717997 | 2.11.91 || KDELC2 | 2.11.91 | KDEL motif containing 2 | 0.344744974 |
2.11.91 || KITLG | 2.11.91 | KIT ligand | 1.097244876 | 2.11.91 || LAPTM4B | 2.11.91 | lysosomal
protein transmembrane 4 beta | 0.602776882 | 2.11.91 || LIPT1 | 2.11.91 | lipoyltransferase 1 |
0.344910038 | 2.11.91 || MAGEH1 | 2.11.91 | MAGE family member H1 | 0.217957682 | 2.11.91 ||
MYO6 | 2.11.91 | myosin VI | 0.6419066 | 2.11.91 || NEDD4 | 2.11.91 | neural precursor cell
expressed, developmentally down-regulated 4, E3 ubiquitin protein ligase | 0.808755476 | 2.11.91 ||
PARM1 | 2.11.91 | prostate androgen-regulated mucin-like protein 1 | 1.023653832 | 2.11.91 || PLS3
| 2.11.91 || plastin 3 | 0.294103102 | 2.11.91 || PRKG1 | 2.11.91 | protein kinase, cGMP-dependent,
type I | 0.774538466 | 2.11.91 || PRMT6 | 2.11.91 | protein arginine methyltransferase 6 |
0.318595128 | 2.11.91 || PTPN7 | 2.11.91 | protein tyrosine phosphatase, non-receptor type 7 |
0.261209531 | 2.11.91 || PTPRG | 2.11.91 | protein tyrosine phosphatase, receptor type G |
0.965505574 | 2.11.91 || RASGRP2 | 2.11.91 | RAS guanyl releasing protein 2 | 0.253098453 |
2.11.91 || SASH1 | 2.11.91 || SAM and SH3 domain containing 1 | 0.619338966 | 2.11.91 || SEC16B
| 2.11.91 || SEC16 homolog B, endoplasmic reticulum export factor | 0.340832577 | 2.11.91 || SELP |
2.11.91 | selectin P | 1.790362768 | 2.11.91 || SMARCA1 | 2.11.91 | SWI/SNF related, matrix
associated, actin dependent regulator of chromatin, subfamily a, member 1 | 0.518225364 | 2.11.91
|| SPOCK1 | 2.11.91 | sparc/osteonectin, cwcv and kazal-like domains proteoglycan (testican) 1 |
1.725208877 | 2.11.91 || TFDP2 | 2.11.91 | transcription factor Dp-2 (E2F dimerization partner 2) |
0.736516249 | 2.11.91 || TMEFF2 | 2.11.91 | transmembrane protein with EGF like and two
follistatin like domains 2 | 0.709341931 | 2.11.91 || VGLL3 | 2.11.91 | vestigial like family member
3 | 1.251616228 | 2.11.91 || ZEB1 | 2.11.91 | zinc finger E-box binding homeobox 1 | 0.734907942 |
2.11.91 || ZNF45 | 2.11.91 | zinc finger protein 45 | 0.255425089 | 2.11.91 || ZNF600 | 2.11.91 | zinc
finger protein 600 | 0.255226768 | 2.11.91 || ZNF608 | 2.11.91 | zinc finger protein 608 |
1.201445355 | 2.11.91 || ZNF827 | 2.11.91 | zinc finger protein 827 | 0.939881938 | 2.11.91 || ABI2 |
2.11.93 | abl-interactor 2 | 0.750557435 | 2.11.93 || ABI3BP | 2.11.93 | ABI family member 3
binding protein | 0.380939275 | 2.11.93 || CEP85 | 2.11.93 | centrosomal protein 85kDa |
0.383549817 | 2.11.93 || CLASP1 | 2.11.93 | cytoplasmic linker associated protein 1 | 0.271094193 |
2.11.93 || DAB2 | 2.11.93 | Dab, mitogen-responsive phosphoprotein, homolog 2 (Drosophila) |
0.438747419 | 2.11.93 || DCBLD2 | 2.11.93 | discoidin, CUB and LCCL domain containing 2 |
0.444713707 | 2.11.93 || DPY19L3 | 2.11.93 | dpy-19 like 3 (C. elegans) | 0.596164066 | 2.11.93 ||
DPY19L4 | 2.11.93 | dpy-19 like 4 (C. elegans) | 0.543100176 | 2.11.93 || DSPP | 2.11.93 | dentin
sialophosphoprotein | 0.260053984 | 2.11.93 || EFCAB7 | 2.11.93 | EF-hand calcium binding
domain 7 | 0.460111073 | 2.11.93 || EXT1 | 2.11.93 | exostosin glycosyltransferase 1 | 0.376437861 |
2.11.93 || FAM179B | 2.11.93 | family with sequence similarity 179 member B | 0.306610698 |
2.11.93 || FANCL | 2.11.93 | Fanconi anemia complementation group L | 0.259473729 | 2.11.93 ||
FLJ20021 | 2.11.93 | uncharacterized LOC90024 | 0.284145227 | 2.11.93 || FNDC3B | 2.11.93 |
fibronectin type III domain containing 3B | 0.227687911 | 2.11.93 || FPR1 | 2.11.93 | formyl peptide
receptor 1 | 0.886015891 | 2.11.93 || FUT5 | 2.11.93 | fucosyltransferase 5 | 0.385385121 | 2.11.93 ||
GAREM1 | 2.11.93 | GRB2 associated regulator of MAPKI subtype 1 | 1.00034877 | 2.11.93 ||
GMFG | 2.11.93 | glia maturation factor gamma | 0.45780896 | 2.11.93 || GOLGA2 | 2.11.93 |
golgin A2 | 0.458222426 | 2.11.93 || GOLIM4 | 2.11.93 | golgi integral membrane protein 4 |
0.322114154 | 2.11.93 || HAUS6 | 2.11.93 | HAUS augmin like complex subunit 6 | 0.560054086 |
2.11.93 || HOXA5 | 2.11.93 | homeobox A5 | 0.886096098 | 2.11.93 || IFT80 | 2.11.93 | intraflagellar
transport 80 | 0.411958681 | 2.11.93 || KCNJ2 | 2.11.93 | potassium voltage-gated channel subfamily
J member 2 | 0.732493091 | 2.11.93 || LOC400043 | 2.11.93 | uncharacterized LOC400043 |
0.328282968 | 2.11.93 || MLLT3 | 2.11.93 | myeloid/lymphoid or mixed-lineage leukemia;
translocated to, 3 | 0.364863776 | 2.11.93 || NOL12 | 2.11.93 | nucleolar protein 12 | 0.284900775 |
2.11.93 || OSMR | 2.11.93 | oncostatin M receptor | 1.503278899 | 2.11.93 || PCDHB14 | 2.11.93 |
protocadherin beta 14 | 1.252426102 | 2.11.93 || PDE10A | 2.11.93 | phosphodiesterase 10A |
0.593443067 | 2.11.93 || SACS | 2.11.93 | sacsin molecular chaperone | 0.485582191 | 2.11.93 ||
SEPT8 | 2.11.93 | septin 8 | 0.267651618 | 2.11.93 || SH3PXD2A | 2.11.93 | SH3 and PX domains
2A | 0.386457248 | 2.11.93 || SLC16A6 | 2.11.93 | solute carrier family 16 member 6 | 1.372102975
| 2.11.93 || SLC5A3 | 2.11.93 | solute carrier family 5 member 3 | 1.125767521 | 2.11.93 ||
SLCO2A1 | 2.11.93 | solute carrier organic anion transporter family member 2A1 | 0.595840495 |
2.11.93 || SLIT2 | 2.11.93 | slit guidance ligand 2 | 1.534824777 | 2.11.93 || TBC1D8B | 2.11.93 |
TBC1 domain family member 8B | 0.93406443 | 2.11.93 || TMEM136 | 2.11.93 | transmembrane
protein 136 | 0.50399416 | 2.11.93 || TRERF1 | 2.11.93 | transcriptional regulating factor 1 |
0.700646116 | 2.11.93 || TRIB1 | 2.11.93 | tribbles pseudokinase 1 | 0.406969487 | 2.11.93 ||
TRIM13 | 2.11.93 | tripartite motif containing 13 | 0.67103634 | 2.11.93 || TRNT1 | 2.11.93 | tRNA
nucleotidyl transferase, CCA-adding, 1 | 0.442827291 | 2.11.93 || TSPAN15 | 2.11.93 | tetraspanin
15 | 0.512575205 | 2.11.93 || TTC28 | 2.11.93 | tetratricopeptide repeat domain 28 | 1.083496716 |
2.11.93 || TTC8 | 2.11.93 | tetratricopeptide repeat domain 8 | 0.856140725 | 2.11.93 || ZNF268 |
2.11.93 | zinc finger protein 268 | 0.89780186 | 2.11.93 || ZNF329 | 2.11.93 | zinc finger protein 329
| 0.232425809 | 2.11.93 || ZNF503 | 2.11.93 | zinc finger protein 503 | 0.935714707 | 2.11.93 ||
ZNF512B | 2.11.93 | zinc finger protein 512B | 0.500698568 | 2.11.93 || ZNF618 | 2.11.93 | zinc
finger protein 618 | 1.07071906 | 2.11.93 || ARNTL2 | 2.11.96 | aryl hydrocarbon receptor nuclear
translocator like 2 | 0.494590322 | 2.11.96 || C7 | 2.11.96 | complement component 7 | 5.235794833 |
2.11.96 || CAMLG | 2.11.96 | calcium modulating ligand | 0.285590664 | 2.11.96 || CFH | 2.11.96 |
complement factor H | 0.713023953 | 2.11.96 || CPE | 2.11.96 | carboxypeptidase E | 1.901786183 |
2.11.96 || DESI1 | 2.11.96 | desumoylating isopeptidase 1 | 0.404636144 | 2.11.96 || EVA1C |
2.11.96 | eva-1 homolog C (C. elegans) | 1.030366337 | 2.11.96 || FAM228B | 2.11.96 | family with
sequence similarity 228 member B | 0.349991862 | 2.11.96 || FBLN5 | 2.11.96 | fibulin 5 |
1.139777302 | 2.11.96 || GPATCH1 | 2.11.96 | G-patch domain containing 1 | 0.219040373 | 2.11.96
| LINC01139 | 2.11.96 | long intergenic non-protein coding RNA 1139 | 0.581493857 | 2.11.96 ||
MAB21L2 | 2.11.96 | mab-21-like 2 (C. elegans) | 4.062089165 | 2.11.96 || MBP | 2.11.96 | myelin
basic protein | 0.578176871 | 2.11.96 || PID1 | 2.11.96 | phosphotyrosine interaction domain
containing 1 | 0.433982727 | 2.11.96 || PRICKLE2 | 2.11.96 | prickle planar cell polarity protein 2 |
0.782184554 | 2.11.96 || SIGLEC9 | 2.11.96 | sialic acid binding Ig like lectin 9 | 0.430433642 |
2.11.96 || SLC2A5 | 2.11.96 | solute carrier family 2 member 5 | 0.855621559 | 2.11.96 ||
TNFRSF21 | 2.11.96 | tumor necrosis factor receptor superfamily member 21 | 0.542314989 |
2.11.96 || WARS2 | 2.11.96 | tryptophanyl tRNA synthetase 2, mitochondrial | 0.70446101 | 2.11.96
|| WLS | 2.11.96 | wntless Wnt ligand secretion mediator | 1.31650603 | 2.11.96 || ZFHX4 | 2.11.96 |
zinc finger homeobox 4 | 1.487261107 | 2.11.96 || ZNF507 | 2.11.96 | zinc finger protein 507|
0.334228087 | 2.11.96 || ABCA6 | 2.12 | ATP binding cassette subfamily A member 6 |
2.446980416 | 2.12.103 | ACE | 2.12 | angiotensin I converting enzyme | 0.822173231 | 2.12.101 ||
ADAP1 | 2.12 | ArfGAP with dual PH domains 1 | 0.227749312 | 2.12.101 || ADGRE5 | 2.12 |
adhesion G protein-coupled receptor E5 | 0.576993351 | 2.12.101 || AGPAT3 | 2.12 | 1-acylglycerol-
3-phosphate O-acyltransferase 3 | 0.326055015 | 2.12.101 || ALCAM | 2.12 | activated leukocyte
cell adhesion molecule | 0.749551732 | 2.12.104 || ALDH5A1 | 2.12 | aldehyde dehydrogenase 5
family member A1 | 0.281576773 | 2.12.103 || ALDH7A1 | 2.12 | aldehyde dehydrogenase 7 family
member Al | 0.234976944 | - || ALDOC | 2.12 | aldolase, fructose-bisphosphate C | 0.943730749 |
2.12.103 || ANGPT1 | 2.12 | angiopoietin 1 | 3.008389668 | 2.12.101 || ANKRD46 | 2.12 | ankyrin
repeat domain 46 | 0.780596259 | 2.12.103 || ANPEP | 2.12 | alanyl aminopeptidase, membrane |
1.337916135 | 2.12.103 || ANXA1 | 2.12 | annexin Al | 0.256970367 | 2.12.106 || APEX2 | 2.12 |
apurinic/apyrimidinic endodeoxyribonuclease 2 | 0.218435554 | 2.12.101 || APOLD1 | 2.12 |
apolipoprotein L domain containing 1 | 1.16275303 | 2.12.107 || ARHGEF10L | 2.12 | Rho guanine
nucleotide exchange factor 10 like | 0.229452534 | 2.12.101 || ARRDC4 | 2.12 | arrestin domain
containing 4 | 0.332199587 | 2.12.103 || ASPN | 2.12 | asporin | 0.761715506 | 2.12.106 || ATG4B |
2.12 | autophagy related 4B cysteine peptidase | 0.256069183 | 2.12.103 || ATP1B1 | 2.12 | ATPase
Na+/K+ transporting subunit beta 1 | 0.607829393 | 2.12.105 || B4GALT1 | 2.12 | UDP-
Gal:betaGlcNAc beta 1,4- galactosyltransferase, polypeptide 1 | 0.544686323 | 2.12.104 || BAG3 |
2.12 | BCL2 associated athanogene 3 | 0.229697872 | 2.12.105 || BAHD1 | 2.12 | bromo adjacent
homology domain containing 1 | 0.266102832 | 2.12.106 || BBIP1 | 2.12 | BBSome interacting
protein 1 | 0.216472744 | 2.12.103 || BBOF1 | 2.12 | basal body orientation factor 1 | 0.324475326 |
2.12.104 || BCAR3 | 2.12 | breast cancer anti-estrogen resistance 3 | 0.297371169 | 2.12.103 || BIN2
| 2.12 | bridging integrator 2 | 0.251622852 | 2.12.101 || C1GALT1 | 2.12 | core 1 synthase,
glycoprotein-N-acetylgalactosamine 3-beta-galactosyltransferase 1 | 1.306472927 | 2.12.104 ||
CACNA2D1 | 2.12 | calcium voltage-gated channel auxiliary subunit alpha2delta 1 | 1.548364794 |
- || CARD19 | 2.12 | caspase recruitment domain family member 19 | 0.234570383 | 2.12.101 ||
CCDC88A | 2.12 | coiled-coil domain containing 88A | 0.223034609 | 2.12.104 || CCL19 | 2.12 | C-
C motif chemokine ligand 19 | 2.41316504 | 2.12.106 || CCND3 | 2.12 | cyclin D3 | 0.366729564 |
2.12.101 || CCNG1 | 2.12 | cyclin G1 | 0.239865274 | - || CCR1 | 2.12 | chemokine (C-C motif)
receptor 1 | 0.715344552 | 2.12.104 || CCR5 | 2.12 | chemokine (C-C motif) receptor 5
(gene/pseudogene) | 1.540711162 | 2.12.104 || CD14 | 2.12 | CD14 molecule | 0.476939467 |
2.12.104 || CD300C | 2.12 | CD300c molecule | 1.120289507 | 2.12.101 || CD4 | 2.12 | CD4
molecule | 0.222176835 | 2.12.104 || CD44 | 2.12 | CD44 molecule (Indian blood group) |
0.250184608 | 2.12.101 || CD86 | 2.12 | CD86 molecule | 0.874437231 | 2.12.107 || CDC34 | 2.12 |
cell division cycle 34 | 0.227013035 | 2.12.106 || CECR1 | 2.12 | cat eye syndrome chromosome
region, candidate 1 | 1.519960814 | 2.12.104 || CHFR | 2.12 | checkpoint with forkhead and ring
finger domains, E3 ubiquitin protein ligase | 0.376419996 | 2.12.101 || CIITA | 2.12 | class II, major
histocompatibility complex, transactivator | 0.408325037 | 2.12.107 || CISD1 | 2.12 | CDGSH iron
sulfur domain 1 | 0.581864792 | 2.12.104 || CLECIA | 2.12 | C-type lectin domain family 1 member
A | 0.292741233 | 2.12.104 || CLEC4E | 2.12 | C-type lectin domain family 4 member E |
1.282428339 | 2.12.101 || CLEC7A | 2.12 | C-type lectin domain family 7 member A | 0.765737967
| 2.12.107 || CMTM7 | 2.12 | CKLF like MARVEL transmembrane domain containing 7 |
0.229634714 | 2.12.103 || CNDP2 | 2.12 | CNDP dipeptidase 2 (metallopeptidase M20 family) |
0.314323478 | 2.12.107 || CNN3 | 2.12 | calponin 3 | 1.57669861 | 2.12.101 || CRIP2 | 2.12 | cysteine
rich protein 2 | 1.358696907 | 2.12.106 || CRTAM | 2.12 | cytotoxic and regulatory T-cell molecule |
0.492695708 | 2.12.107 | CSF1R | 2.12 | colony stimulating factor 1 receptor | 0.521530062 |
2.12.104 || CSK | 2.12 | c-src tyrosine kinase | 0.421471419 | 2.12.101 || CSTB | 2.12 | cystatin B |
0.662075204 | 2.12.103 || CTSA | 2.12 | cathepsin A | 0.410870922 | 2.12.106 || CTSS | 2.12 |
cathepsin S | 0.293650137 | 2.12.107 || CXCL16 | 2.12 | C-X-C motif chemokine ligand 16 |
0.961606521 | 2.12.104 || CYP27A1 | 2.12 | cytochrome P450 family 27 subfamily A member 1 |
0.575476 | 2.12.103 || CYTH1 | 2.12 | cytohesin 1 | 0.281087033 | 2.12.104 || DBN1 | 2.12 | drebrin
1 | 0.359697531 | 2.12.107 || DCUNID1 | 2.12 | defective in cullin neddylation 1 domain containing
1 | 0.27787274 | 2.12.101 || DDIT4 | 2.12 | DNA damage inducible transcript 4 | 1.002173711 |
2.12.105 || DEF6 | 2.12 | DEF6, guanine nucleotide exchange factor | 0.256345421 | 2.12.104 ||
DENND5A | 2.12 | DENN domain containing 5A | 0.238081052 | 2.12.101 || DENND5B | 2.12 |
DENN domain containing 5B | 0.686236457 | 2.12.104 || DHRS9 | 2.12 | dehydrogenase/reductase
(SDR family) member 9 | 1.280820142 | 2.12.104 || DMD | 2.12 | dystrophin | 3.102263099 |
2.12.103 || DNAJB4 | 2.12 | DnaJ heat shock protein family (Hsp40) member B4 | 0.328615897 |
2.12.107 || DPEP2 | 2.12 | dipeptidase 2 | 1.720455863 | 2.12.104 || DSC2 | 2.12 | desmocollin 2 |
0.25527513 | 2.12.107 || DUXAP10 | 2.12 | double homeobox A pseudogene 10 | 2.713198962 |
2.12.101 || ECM1 | 2.12 | extracellular matrix protein 1 | 0.928792043 | 2.12.103 || EFNA5 | 2.12 |
ephrin-A5 | 0.949534783 | 2.12.104 || EMILIN2 | 2.12 | elastin microfibril interfacer 2 |
0.846147238 | - || ENO2 | 2.12 | enolase 2 (gamma, neuronal) | 1.925091902 | 2.12.101 || ESF1 |
2.12 | ESF1 nucleolar pre-rRNA processing protein homolog | 0.694909511 | 2.12.101 || EVI2B |
2.12 | ecotropic viral integration site 2B | 0.442802766 | 2.12.104 || EZR | 2.12 | ezrin | 0.35464123 |
2.12.103 || FAM217B | 2.12 | family with sequence similarity 217 member B | 0.408663559 |
2.12.105 || FAM76B | 2.12 | family with sequence similarity 76 member B | 0.241659847 | 2.12.103
|| FAR2 | 2.12 | fatty acyl-CoA reductase 2 | 0.977900947 | 2.12.101 || FBXL4 | 2.12 | F-box and
leucine-rich repeat protein 4 | 0.529242019 | 2.12.104 || FBXO3 | 2.12 | F-box protein 3 |
0.568917163 | 2.12.105 || FGF7 | 2.12 | fibroblast growth factor 7 | 1.359856902 | 2.12.101 || FGR |
2.12 | FGR proto-oncogene, Src family tyrosine kinase | 2.300021414 | 2.12.104 || FILIP1 | 2.12 |
filamin A interacting protein 1 | 2.150542453 | 2.12.105 || FOXP4 | 2.12 | forkhead box P4 |
0.361538465 | 2.12.106 || FRAT1 | 2.12 | frequently rearranged in advanced T-cell lymphomas 1 |
0.506487654 | 2.12.101 || FXYD1 | 2.12 | FXYD domain containing ion transport regulator 1 |
0.938165093 | - || FXYD2 | 2.12 | FXYD domain containing ion transport regulator 2 | 0.440705488
| 2.12.101 || FYCO1 | 2.12 | FYVE and coiled-coil domain containing 1 | 0.569072965 | - ||
GABRB1 | 2.12 | gamma-aminobutyric acid type A receptor betal subunit | 2.96219369 | 2.12.104 ||
GALNT12 | 2.12 | polypeptide N-acetylgalactosaminyltransferase 12 | 1.09110838 | 2.12.101 ||
GALNT6 | 2.12 | polypeptide N-acetylgalactosaminyltransferase 6 | 0.588788908 | 2.12.107 ||
GAS2L1 | 2.12 | growth arrest specific 2 like 1 | 0.602767108 | 2.12.106 || GGCX | 2.12 | gamma-
glutamyl carboxylase | 0.273747239 | 2.12.103 || GLMP | 2.12 | glycosylated lysosomal membrane
protein | 0.243880069 | 2.12.103 || GPCPD1 | 2.12 | glycerophosphocholine phosphodiesterase 1 |
0.576944971 | 2.12.103 || GPR137B | 2.12 | G protein-coupled receptor 137B | 0.253109168 |
2.12.103 || GRB2 | 2.12 | growth factor receptor bound protein 2 | 0.331028902 | 2.12.107 || GRINA
| 2.12 | glutamate ionotropic receptor NMDA type subunit associated protein 1 | 0.350327141 |
2.12.104 || GTPBP8 | 2.12 | GTP-binding protein 8 (putative) | 0.475705316 | 2.12.101 || H1F0 |
2.12 | Hl histone family member 0 | 0.364999083 | 2.12.106 || H2AFY | 2.12 | H2A histone family
member Y | 0.440754871 | 2.12.104 || HAS2 | 2.12 | hyaluronan synthase 2 | 1.207932395 | 2.12.104
|| HIBCH | 2.12 | 3-hydroxyisobutyryl-CoA hydrolase | 0.488970484 | 2.12.103 || HLA-J | 2.12 |
major histocompatibility complex, class I, J (pseudogene) | 0.504591289 | 2.12.101 || HMHA1 |
2.12 | histocompatibility (minor) HA-1 | 0.596715737 | 2.12.104 || HPCAL1 | 2.12 | hippocalcin like
1 | 0.496071858 | 2.12.101 || HRH1 | 2.12 | histamine receptor H1 | 0.48659057 | 2.12.104 || HSPA2
| 2.12 | heat shock protein family A (Hsp70) member 2 | 1.229622178 | - || HSPB2 | 2.12 | heat
shock protein family B (small) member 2 | 0.699781793 | - || HSPB8 | 2.12 | heat shock protein
family B (small) member 8 | 1.289429847 | - || IFT140 | 2.12 | intraflagellar transport 140 |
0.229390913 | 2.12.104 || IFT88 | 2.12 | intraflagellar transport 88 | 0.330102919 | 2.12.105 ||
IGFBP3 | 2.12 | insulin like growth factor binding protein 3 | 0.691039618 | - || IL10RA | 2.12 |
interleukin 10 receptor subunit alpha | 0.57834018 | 2.12.104 || ILK | 2.12 | integrin linked kinase |
0.246792144 | 2.12.106 || IMMPIL | 2.12 | inner mitochondrial membrane peptidase subunit 1 |
0.406962095 | 2.12.105 || INHBC | 2.12 | inhibin beta C | 0.264875814 | - || ITGB2 | 2.12 | integrin
subunit beta 2 | 1.491347188 | 2.12.104 || JUP | 2.12 | junction plakoglobin | 0.73926426 | 2.12.101 ||
KCNAB2 | 2.12 | potassium voltage-gated channel subfamily A regulatory beta subunit 2 |
1.204761556 | 2.12.104 || KCNMB1 | 2.12 | potassium calcium-activated channel subfamily M
regulatory beta subunit 1 | 0.62959806 | - || KDM1B | 2.12 | lysine demethylase 1B | 0.571725926 | -
|| KDM3A | 2.12 | lysine demethylase 3A | 0.534088404 | 2.12.101 || KIAA0513 | 2.12 | KIAA0513 |
0.307126608 | 2.12.101 | KIFIB | 2.12 | kinesin family member 1B | 0.413102517 | 2.12.105 | KIF9
| 2.12 | kinesin family member 9 | 0.226814631 | 2.12.105 || LBX2-AS1 | 2.12 | LBX2 antisense
RNA 1 | 0.246214046 | 2.12.101 || LGALSL | 2.12 | lectin, galactoside-binding-like | 0.698144253 |
2.12.105 || LHFPL2 | 2.12 | lipoma HMGIC fusion partner-like 2 | 0.49592026 | 2.12.103 ||
LINC00894 | 2.12 | long intergenic non-protein coding RNA 894 | 0.481509831 | 2.12.104 ||
LOC154761 | 2.12 | family with sequence similarity 115, member C pseudogene | 1.412367117 |
2.12.103 || LOC374443 | 2.12 | C-type lectin domain family 2 member D pseudogene | 0.441781327
| 2.12.101 || LONP1 | 2.12 | lon peptidase 1, mitochondrial | 0.243949601 | 2.12.101 || LPCAT2 |
2.12 | lysophosphatidylcholine acyltransferase 2 | 0.728406381 | 2.12.106 || LRP5 | 2.12 | LDL
receptor related protein 5 | 0.324201869 | 2.12.107 || LZTFL1 | 2.12 | leucine zipper transcription
factor like 1 | 0.306767959 | 2.12.105 || MANBA | 2.12 | mannosidase beta | 0.265479243 | 2.12.103
|| MAP7D1 | 2.12 | MAP7 domain containing 1 | 0.21489017 | 2.12.101 || MARCH1 | 2.12 |
membrane associated ring-CH-type finger 1 | 0.664350399 | 2.12.107 || MEIS2 | 2.12 | Meis
homeobox 2 | 1.453497642 | 2.12.101 || MERTK | 2.12 | MER proto-oncogene, tyrosine kinase |
1.090440676 | 2.12.103 || MGA | 2.12 | MGA, MAX dimerization protein | 0.345347405 | 2.12.104
|| MID1 | 2.12 | midline 1 | 1.014800351 | 2.12.103 || MIDN | 2.12 | midnolin | 0.397682982 | - ||
MIR146A | 2.12 | microRNA 146a | 0.290852577 | 2.12.103 || MPP1 | 2.12 | membrane protein,
palmitoylated 1 | 0.288529638 | 2.12.101 || MPP5 | 2.12 | membrane protein, palmitoylated 5 |
0.241556347 | 2.12.105 || MREG | 2.12 | melanoregulin | 1.498263672 | 2.12.107 || MRPL19 | 2.12 |
mitochondrial ribosomal protein L19 | 0.247269123 | 2.12.104 || MYADM | 2.12 | myeloid-
associated differentiation marker | 0.667791803 | 2.12.106 || MYC | 2.12 | v-myc avian
myelocytomatosis viral oncogene homolog | 0.346971875 | 2.12.107 || MYD88 | 2.12 | myeloid
differentiation primary response 88 | 0.599349644 | 2.12.101 || MYO1E | 2.12 | myosin IE |
0.260241201 | 2.12.101 || MYOF | 2.12 | myoferlin | 0.285690144 | 2.12.106 || NAA15 | 2.12 |
N(alpha)-acetyltransferase 15, NatA auxiliary subunit | 0.390736172 | 2.12.101 || NANOS1 | 2.12 |
nanos homolog 1 (Drosophila) | 0.54898783 | 2.12.103 || NCF2 | 2.12 | neutrophil cytosolic factor 2 |
1.048558482 | 2.12.101 || NCKIPSD | 2.12 | NCK interacting protein with SH3 domain |
0.344097509 | 2.12.106 || NDRG1 | 2.12 | N-myc downstream regulated 1 | 0.439750954 | 2.12.103
|| NECTIN3 | 2.12 | nectin cell adhesion molecule 3 | 0.41568067 | 2.12.105 || NFKBIZ | 2.12 |
NFKB inhibitor zeta | 0.371426958 | - || NOP9 | 2.12 | NOP9 nucleolar protein | 0.322998316 |
2.12.104 || OSBPL3 | 2.12 | oxysterol binding protein like 3 | 0.292025729 | 2.12.107 || OTUD1 |
2.12 | OTU deubiquitinase 1 | 0.432181994 | 2.12.103 || P2RX7 | 2.12 | purinergic receptor P2X 7 |
1.410038537 | 2.12.107 || PAPSS2 | 2.12 | 3′-phosphoadenosine 5′-phosphosulfate synthase 2 |
0.356985586 | 2.12.103 || PATL1 | 2.12 | protein associated with topoisomerase II homolog 1
(yeast) | 0.451781219 | 2.12.101 || PAWR | 2.12 | pro-apoptotic WT1 regulator | 1.674403695 |
2.12.103 || PEG3 | 2.12 | paternally expressed 3 | 0.62981622 | 2.12.107 | PELI1 | 2.12 | pellino E3
ubiquitin protein ligase 1 | 0.348654689 | 2.12.104 || PFKP | 2.12 | phosphofructokinase, platelet |
0.702107291 | 2.12.106 || PGD | 2.12 | phosphogluconate dehydrogenase | 0.53732397 | 2.12.101 ||
PIK3C2B | 2.12 | phosphatidylinositol-4-phosphate 3-kinase catalytic subunit type 2 beta |
0.63483958 | 2.12.103 || PLK3 | 2.12 | polo like kinase 3 | 0.406159757 | 2.12.103 || PNMA2 | 2.12 |
paraneoplastic Ma antigen 2 | 0.863778695 | 2.12.104 || PNO1 | 2.12 | partner of NOB1 homolog |
0.369837084 | 2.12.101 || PNPLA6 | 2.12 | patatin like phospholipase domain containing 6 |
0.461952597 | - || POGK | 2.12 | pogo transposable element with KRAB domain | 0.225286897 |
2.12.101 || PPM1M | 2.12 | protein phosphatase, Mg2+/Mn2+ dependent 1M | 1.091444897 |
2.12.106 || PTCH1 | 2.12 | patched 1 | 0.699706032 | 2.12.107 || PTGFR | 2.12 | prostaglandin F
receptor | 2.888425389 | 2.12.101 || PTK2B | 2.12 | protein tyrosine kinase 2 beta | 1.103557067 |
2.12.104 || PTPN6 | 2.12 | protein tyrosine phosphatase, non-receptor type 6 | 0.589655517 |
2.12.104 || QPCT | 2.12 | glutaminyl-peptide cyclotransferase | 1.625708384 | 2.12.101 || RAB11B |
2.12 | RAB11B, member RAS oncogene family | 0.45922951 | 2.12.101 || RAB28 | 2.12 | RAB28,
member RAS oncogene family | 0.271338912 | 2.12.105 || RAB31 | 2.12 | RAB31, member RAS
oncogene family | 0.215426784 | 2.12.107 || RAB8A | 2.12 | RAB8A, member RAS oncogene
family | 0.230331134 | 2.12.101 || RAPGEF4 | 2.12 | Rap guanine nucleotide exchange factor 4 |
0.966494078 | - || RARRES1 | 2.12 | retinoic acid receptor responder (tazarotene induced) 1 |
2.495773677 | 2.12.101 || RCSD1 | 2.12 | RCSD domain containing 1 | 0.560084528 | 2.12.104 ||
RHBDF2 | 2.12 | rhomboid 5 homolog 2 (Drosophila) | 0.712330442 | 2.12.104 || RHOBTB1 | 2.12 |
Rho related BTB domain containing 1 | 0.310172604 | - || RIN3 | 2.12 | Ras and Rab interactor 3 |
0.471907039 | 2.12.103 || RNF187 | 2.12 | ring finger protein 187 | 0.369042429 | 2.12.106 ||
RNF219 | 2.12 | ring finger protein 219 | 0.253617613 | 2.12.103 || RPRDIA | 2.12 | regulation of
nuclear pre-mRNA domain containing 1A | 0.367871682 | 2.12.103 || RUNDC3B | 2.12 | RUN
domain containing 3B | 0.455915526 | - || S100A10 | 2.12 | S100 calcium binding protein A10 |
0.221750942 | 2.12.101 || S100A11 | 2.12 | S100 calcium binding protein All | 0.313350115 |
2.12.103 || SCFD2 | 2.12 | sec1 family domain containing 2 | 0.269780851 | 2.12.101 || SELPLG |
2.12 | selectin P ligand | 0.446154855 | 2.12.104 | SGCB | 2.12 | sarcoglycan beta | 0.519046643 |
2.12.105 || SGK1 | 2.12 | serum/glucocorticoid regulated kinase 1 | 0.671092889 | 2.12.103 ||
SGMS2 | 2.12 | sphingomyelin synthase 2 | 0.568789822 | 2.12.103 || SIRPB1 | 2.12 | signal
regulatory protein beta 1 | 0.780891036 | 2.12.103 || SIRT7 | 2.12 | sirtuin 7 | 0.233507051 |
2.12.103 || SIX1 | 2.12 | SIX homeobox 1 | 1.031010244 | 2.12.105 || SLC22A15 | 2.12 | solute
carrier family 22 member 15 | 0.414116218 | 2.12.103 || SLC31A2 | 2.12 | solute carrier family 31
member 2 | 1.086103798 | 2.12.104 || SLC43A3 | 2.12 | solute carrier family 43 member 3 |
0.404004154 | 2.12.104 || SLC6A8 | 2.12 | solute carrier family 6 member 8 | 1.164768002 |
2.12.101 || SLC8A1 | 2.12 | solute carrier family 8 member Al | 0.382467359 | 2.12.107 || SLC8B1 |
2.12 | solute carrier family 8 member B1 | 0.215308981 | 2.12.103 || SP4 | 2.12 | Sp4 transcription
factor | 0.363196732 | 2.12.103 || SPINT1 | 2.12 | serine peptidase inhibitor, Kunitz type 1 |
0.409337336 | 2.12.103 || SPP1 | 2.12 | secreted phosphoprotein 1 | 4.314548464 | 2.12.105 ||
SPRY1 | 2.12 | sprouty RTK signaling antagonist 1 | 1.075125941 | 2.12.107 || SPTLC2 | 2.12 |
serine palmitoyltransferase long chain base subunit 2 | 0.461048192 | 2.12.101 || SRC | 2.12 | SRC
proto-oncogene, non-receptor tyrosine kinase | 0.703451349 | 2.12.106 || SRXN1 | 2.12 |
sulfiredoxin 1 | 0.245431157 | 2.12.106 || ST14 | 2.12 | suppression of tumorigenicity 14 |
0.668616128 | 2.12.105 || ST3GAL1 | 2.12 | ST3 beta-galactoside alpha-2,3-sialyltransferase 1 |
0.378196061 | 2.12.101 || STAB1 | 2.12 | stabilin 1 | 0.273598284 | 2.12.103 || STEAP3 | 2.12 |
STEAP3 metalloreductase | 0.884953473 | 2.12.103 || STXBP2 | 2.12 | syntaxin binding protein 2 |
0.847787556 | 2.12.101 || SULF2 | 2.12 | sulfatase 2 | 0.426220136 | 2.12.101 || SUSD6 | 2.12 | sushi
domain containing 6 | 0.380056742 | 2.12.104 || SVIL | 2.12 | supervillin | 0.812893235 | - || SVIP |
2.12 | small VCP/p97-interacting protein | 0.748015774 | 2.12.103 || SYK | 2.12 | spleen tyrosine
kinase | 0.499574814 | 2.12.104 || TACC2 | 2.12 | transforming acidic coiled-coil containing protein
2 | 0.266123476 | - || TAGLN2 | 2.12 | transgelin 2 | 0.412705258 | 2.12.106 || TBC1D4 | 2.12 |
TBC1 domain family member 4 | 0.40550098 | 2.12.103 || TBCK | 2.12 | TBC1 domain containing
kinase | 0.29938482 | 2.12.101 || TCF7 | 2.12 | transcription factor 7 (T-cell specific, HMG-box) |
0.503769999 | 2.12.103 || TCIRG1 | 2.12 | T-cell immune regulator 1, ATPase H+ transporting V0
subunit a3 | 0.594879156 | 2.12.103 || TCN2 | 2.12 | transcobalamin 2 | 0.391362798 | 2.12.104 ||
TGFBI | 2.12 | transforming growth factor beta induced | 0.614476288 | 2.12.103 || THEMIS2 | 2.12
| thymocyte selection associated family member 2 | 0.893339303 | 2.12.101 || TMCC3 | 2.12 |
transmembrane and coiled-coil domain family 3 | 0.975389749 | 2.12.103 || TMEM43 | 2.12 |
transmembrane protein 43 | 0.309809559 | 2.12.106 || TREM1 | 2.12 | triggering receptor expressed
on myeloid cells 1 | 3.022587711 | 2.12.103 || TSPAN17 | 2.12 | tetraspanin 17 | 0.241860003 |
2.12.101 || TUBB3 | 2.12 | tubulin beta 3 class III | 0.223478301 | 2.12.106 || TUBGCP2 | 2.12 |
tubulin gamma complex associated protein 2 | 0.396993298 | 2.12.103 || TWF2 | 2.12 | twinfilin
actin binding protein 2 | 0.30778344 | 2.12.103 || UNC5B | 2.12 | unc-5 netrin receptor B |
1.104647732 | 2.12.104 || USP46 | 2.12 | ubiquitin specific peptidase 46 | 0.842920331 | 2.12.104 ||
UTS2 | 2.12 | urotensin 2 | 0.355683143 | - || VAC14 | 2.12 | Vac14 homolog (S. cerevisiae) |
0.308091951 | 2.12.106 || WDFY3-AS2 | 2.12 | WDFY3 antisense RNA 2 | 0.448186957 | 2.12.105
|| YBX3 | 2.12 | Y-box binding protein 3 | 0.416102805 | 2.12.105 || YIF1B | 2.12 | Yip1 interacting
factor homolog B, membrane trafficking protein | 0.376639455 | 2.12.103 || ZAK | 2.12 | sterile
alpha motif and leucine zipper containing kinase AZK | 0.638478903 | - || ZHX2 | 2.12 | zinc fingers
and homeoboxes 2 | 0.512369898 | 2.12.105 || ZMYM5 | 2.12 | zinc finger MYM-type containing 5 |
0.664464766 | 2.12.101 || ZNF124 | 2.12 | zinc finger protein 124 | 0.715444937 | 2.12.101 ||
ZNF304 | 2.12 | zinc finger protein 304 | 0.226046247 | 2.12.103 || ZNF385A | 2.12 | zinc finger
protein 385A | 0.765763932 | 2.12.104 || ZNF616 | 2.12 | zinc finger protein 616 | 0.267943177 | - ||
ZNF687 | 2.12 | zinc finger protein 687 | 0.305127493 | 2.12.101 || ZNF91 | 2.12 | zinc finger
protein 91 | 0.856868546 | 2.12.103 || ACE | 2.12.101 | angiotensin I converting enzyme |
0.822173231 | 2.12.101 || ADAP1 | 2.12.101 | ArfGAP with dual PH domains 1 | 0.227749312 |
2.12.101 || ADGRE5 | 2.12.101 | adhesion G protein-coupled receptor E5 | 0.576993351 | 2.12.101 ||
AGPAT3 | 2.12.101 | 1-acylglycerol-3-phosphate O-acyltransferase 3 | 0.326055015 | 2.12.101 ||
ANGPT1 | 2.12.101 | angiopoietin 1 | 3.008389668 | 2.12.101 || APEX2 | 2.12.101 |
apurinic/apyrimidinic endodeoxyribonuclease 2 | 0.218435554 | 2.12.101 || ARHGEF10L | 2.12.101
| Rho guanine nucleotide exchange factor 10 like | 0.229452534 | 2.12.101 || BIN2 | 2.12.101 |
bridging integrator 2 | 0.251622852 | 2.12.101 || CARD19 | 2.12.101 | caspase recruitment domain
family member 19 | 0.234570383 | 2.12.101 || CCND3 | 2.12.101 | cyclin D3 | 0.366729564 |
2.12.101 || CD300C | 2.12.101 | CD300c molecule | 1.120289507 | 2.12.101 || CD44 | 2.12.101 |
CD44 molecule (Indian blood group) | 0.250184608 | 2.12.101 || CHFR | 2.12.101 | checkpoint with
forkhead and ring finger domains, E3 ubiquitin protein ligase | 0.376419996 | 2.12.101 || CLEC4E |
2.12.101 | C-type lectin domain family 4 member E | 1.282428339 | 2.12.101 || CNN3 | 2.12.101 |
calponin 3 | 1.57669861 | 2.12.101 || CSK | 2.12.101 | c-src tyrosine kinase | 0.421471419 | 2.12.101
|| DCUNID1 | 2.12.101 | defective in cullin neddylation 1 domain containing 1 | 0.27787274 |
2.12.101 || DENND5A | 2.12.101 | DENN domain containing 5A | 0.238081052 | 2.12.101 ||
DUXAP10 | 2.12.101 | double homeobox A pseudogene 10 | 2.713198962 | 2.12.101 || ENO2 |
2.12.101 | enolase 2 (gamma, neuronal) | 1.925091902 | 2.12.101 || ESF1 | 2.12.101 | ESF1
nucleolar pre-rRNA processing protein homolog | 0.694909511 | 2.12.101 || FAR2 | 2.12.101 | fatty
acyl-CoA reductase 2 | 0.977900947 | 2.12.101 || FGF7 | 2.12.101 | fibroblast growth factor 7 |
1.359856902 | 2.12.101 || FRAT1 | 2.12.101 | frequently rearranged in advanced T-cell lymphomas
1 | 0.506487654 | 2.12.101 || FXYD2 | 2.12.101 | FXYD domain containing ion transport regulator 2
| 0.440705488 | 2.12.101 || GALNT12 | 2.12.101 | polypeptide N-acetylgalactosaminyltransferase 12
| 1.09110838 | 2.12.101 || GTPBP8 | 2.12.101 | GTP-binding protein 8 (putative) | 0.475705316 |
2.12.101 || HLA-J | 2.12.101 | major histocompatibility complex, class I, J (pseudogene) |
0.504591289 | 2.12.101 || HPCAL1 | 2.12.101 | hippocalcin like 1 | 0.496071858 | 2.12.101 || JUP |
2.12.101 | junction plakoglobin | 0.73926426 | 2.12.101 || KDM3A | 2.12.101 | lysine demethylase
3A | 0.534088404 | 2.12.101 || KIAA0513 | 2.12.101 | KIAA0513 | 0.307126608 | 2.12.101 || LBX2-
AS1 | 2.12.101 | LBX2 antisense RNA 1 | 0.246214046 | 2.12.101 || LOC374443 | 2.12.101 | C-type
lectin domain family 2 member D pseudogene | 0.441781327 | 2.12.101 || LONP1 | 2.12.101 | lon
peptidase 1, mitochondrial | 0.243949601 | 2.12.101 || MAP7D1 | 2.12.101 | MAP7 domain
containing 1 | 0.21489017 | 2.12.101 || MEIS2 | 2.12.101 | Meis homeobox 2 | 1.453497642 |
2.12.101 || MPP1 | 2.12.101 | membrane protein, palmitoylated 1 | 0.288529638 | 2.12.101 ||
MYD88 | 2.12.101 | myeloid differentiation primary response 88 | 0.599349644 | 2.12.101 ||
MYO1E | 2.12.101 | myosin IE | 0.260241201 | 2.12.101 || NAA15 | 2.12.101 | N(alpha)-
acetyltransferase 15, NatA auxiliary subunit | 0.390736172 | 2.12.101 || NCF2 | 2.12.101 | neutrophil
cytosolic factor 2 | 1.048558482 | 2.12.101 || PATL1 | 2.12.101 | protein associated with
topoisomerase II homolog 1 (yeast) | 0.451781219 | 2.12.101 || PGD | 2.12.101 | phosphogluconate
dehydrogenase | 0.53732397 | 2.12.101 || PNO1 | 2.12.101 | partner of NOB1 homolog |
0.369837084 | 2.12.101 || POGK | 2.12.101 | pogo transposable element with KRAB domain |
0.225286897 | 2.12.101 || PTGFR | 2.12.101 | prostaglandin F receptor | 2.888425389 | 2.12.101 ||
QPCT | 2.12.101 | glutaminyl-peptide cyclotransferase | 1.625708384 | 2.12.101 || RAB11B |
2.12.101 | RAB11B, member RAS oncogene family | 0.45922951 | 2.12.101 || RAB8A | 2.12.101 |
RAB8A, member RAS oncogene family | 0.230331134 | 2.12.101 || RARRES1 | 2.12.101 | retinoic
acid receptor responder (tazarotene induced) 1 | 2.495773677 | 2.12.101 || S100A10 | 2.12.101 |
S100 calcium binding protein A10 | 0.221750942 | 2.12.101 || SCFD2 | 2.12.101 | sec1 family
domain containing 2 | 0.269780851 | 2.12.101 || SLC6A8 | 2.12.101 | solute carrier family 6 member
8 | 1.164768002 | 2.12.101 || SPTLC2 | 2.12.101 | serine palmitoyltransferase long chain base
subunit 2 | 0.461048192 | 2.12.101 || ST3GAL1 | 2.12.101 | ST3 beta-galactoside alpha-2,3-
sialyltransferase 1 | 0.378196061 | 2.12.101 || STXBP2 | 2.12.101 | syntaxin binding protein 2 |
0.847787556 | 2.12.101 || SULF2 | 2.12.101 | sulfatase 2 | 0.426220136 | 2.12.101 || TBCK |
2.12.101 | TBC1 domain containing kinase | 0.29938482 | 2.12.101 || THEMIS2 | 2.12.101 |
thymocyte selection associated family member 2 | 0.893339303 | 2.12.101 || TSPAN17 | 2.12.101 |
tetraspanin 17 | 0.241860003 | 2.12.101 || ZMYM5 | 2.12.101 | zinc finger MYM-type containing 5 |
0.664464766 | 2.12.101 || ZNF124 | 2.12.101 | zinc finger protein 124 | 0.715444937 | 2.12.101 ||
ZNF687 | 2.12.101 | zinc finger protein 687 | 0.305127493 | 2.12.101 || ABCA6 | 2.12.103 | ATP
binding cassette subfamily A member 6 | 2.446980416 | 2.12.103 || ALDH5A1 | 2.12.103 | aldehyde
dehydrogenase 5 family member Al | 0.281576773 | 2.12.103 || ALDOC | 2.12.103 | aldolase,
fructose-bisphosphate C | 0.943730749 | 2.12.103 || ANKRD46 | 2.12.103 | ankyrin repeat domain
46 | 0.780596259 | 2.12.103 || ANPEP | 2.12.103 | alanyl aminopeptidase, membrane | 1.337916135
| 2.12.103 || ARRDC4 | 2.12.103 | arrestin domain containing 4 | 0.332199587 | 2.12.103 || ATG4B |
2.12.103 | autophagy related 4B cysteine peptidase | 0.256069183 | 2.12.103 || BBIP1 | 2.12.103 |
BBSome interacting protein 1 | 0.216472744 | 2.12.103 || BCAR3 | 2.12.103 | breast cancer anti-
estrogen resistance 3 | 0.297371169 | 2.12.103 || CMTM7 | 2.12.103 | CKLF like MARVEL
transmembrane domain containing 7 | 0.229634714 | 2.12.103 || CSTB | 2.12.103 | cystatin B |
0.662075204 | 2.12.103 || CYP27A1 | 2.12.103 | cytochrome P450 family 27 subfamily A member 1
| 0.575476 | 2.12.103 || DMD | 2.12.103 | dystrophin | 3.102263099 | 2.12.103 || ECM1 | 2.12.103 |
extracellular matrix protein 1 | 0.928792043 | 2.12.103 || EZR | 2.12.103 | ezrin | 0.35464123 |
2.12.103 || FAM76B | 2.12.103 | family with sequence similarity 76 member B | 0.241659847
2.12.103 ||| GGCX | 2.12.103 | gamma-glutamyl carboxylase | 0.273747239 | 2.12.103 || GLMP |
2.12.103 | glycosylated lysosomal membrane protein | 0.243880069 | 2.12.103 || GPCPD1 | 2.12.103
| glycerophosphocholine phosphodiesterase 1 | 0.576944971 | 2.12.103 || GPR137B | 2.12.103 | G
protein-coupled receptor 137B | 0.253109168 | 2.12.103 || HIBCH | 2.12.103 | 3-hydroxyisobutyryl-
CoA hydrolase | 0.488970484 | 2.12.103 || LHFPL2 | 2.12.103 | lipoma HMGIC fusion partner-like
2 | 0.49592026 | 2.12.103 || LOC154761 | 2.12.103 | family with sequence similarity 115, member C
pseudogene | 1.412367117 | 2.12.103 || MANBA | 2.12.103 | mannosidase beta | 0.265479243 |
2.12.103 || MERTK | 2.12.103 | MER proto-oncogene, tyrosine kinase | 1.090440676 | 2.12.103 ||
MID1 | 2.12.103 | midline 1 | 1.014800351 | 2.12.103 || MIR146A | 2.12.103 | microRNA 146a |
0.290852577 | 2.12.103 || NANOS1 | 2.12.103 | nanos homolog 1 (Drosophila) | 0.54898783 |
2.12.103 || NDRG1 | 2.12.103 | N-myc downstream regulated 1 | 0.439750954 | 2.12.103 || OTUD1 |
2.12.103 | OTU deubiquitinase 1 | 0.432181994 | 2.12.103 || PAPSS2 | 2.12.103 | 3′-
phosphoadenosine 5′-phosphosulfate synthase 2 | 0.356985586 | 2.12.103 || PAWR | 2.12.103 | pro-
apoptotic WTI regulator | 1.674403695 | 2.12.103 || PIK3C2B | 2.12.103 | phosphatidylinositol-4-
phosphate 3-kinase catalytic subunit type 2 beta | 0.63483958 | 2.12.103 || PLK3 | 2.12.103 | polo
like kinase 3 | 0.406159757 | 2.12.103 || RIN3 | 2.12.103 | Ras and Rab interactor 3 | 0.471907039 |
2.12.103 || RNF219 | 2.12.103 | ring finger protein 219 | 0.253617613 | 2.12.103 || RPRDIA |
2.12.103 | regulation of nuclear pre-mRNA domain containing 1A | 0.367871682 | 2.12.103 ||
S100A11 | 2.12.103 | S100 calcium binding protein All | 0.313350115 | 2.12.103 || SGK1 |
2.12.103 | serum/glucocorticoid regulated kinase 1 | 0.671092889 | 2.12.103 || SGMS2 | 2.12.103 |
sphingomyelin synthase 2 | 0.568789822 | 2.12.103 || SIRPB1 | 2.12.103 | signal regulatory protein
beta 1 | 0.780891036 | 2.12.103 | SIRT7 | 2.12.103 | sirtuin 7 | 0.233507051 | 2.12.103| | SLC22A15
| 2.12.103 | solute carrier family 22 member 15 | 0.414116218 | 2.12.103 || SLC8B1 | 2.12.103 |
solute carrier family 8 member B1 | 0.215308981 | 2.12.103 || SP4 | 2.12.103 | Sp4 transcription
factor | 0.363196732 | 2.12.103 || SPINT1 | 2.12.103 | serine peptidase inhibitor, Kunitz type 1 |
0.409337336 | 2.12.103 || STAB1 | 2.12.103 | stabilin 1 | 0.273598284 | 2.12.103 || STEAP3 |
2.12.103 | STEAP3 metalloreductase | 0.884953473 | 2.12.103 || SVIP | 2.12.103 | small VCP/p97-
interacting protein | 0.748015774 | 2.12.103 || TBC1D4 | 2.12.103 | TBC1 domain family member 4
| 0.40550098 | 2.12.103 || TCF7 | 2.12.103 | transcription factor 7 (T-cell specific, HMG-box) |
0.503769999 | 2.12.103 || TCIRG1 | 2.12.103 | T-cell immune regulator 1, ATPase H+ transporting
V0 subunit a3 | 0.594879156 | 2.12.103 || TGFBI | 2.12.103 | transforming growth factor beta
induced | 0.614476288 | 2.12.103 || TMCC3 | 2.12.103 | transmembrane and coiled-coil domain
family 3 | 0.975389749 | 2.12.103 || TREM1 | 2.12.103 | triggering receptor expressed on myeloid
cells 1 | 3.022587711 | 2.12.103 || TUBGCP2 | 2.12.103 | tubulin gamma complex associated protein
2 | 0.396993298 | 2.12.103 || TWF2 | 2.12.103 | twinfilin actin binding protein 2 | 0.30778344 |
2.12.103 || YIF1B | 2.12.103 | Yip1 interacting factor homolog B, membrane trafficking protein |
0.376639455 | 2.12.103 || ZNF304 | 2.12.103 | zinc finger protein 304 | 0.226046247 | 2.12.103 ||
ZNF91 | 2.12.103 | zinc finger protein 91 | 0.856868546 | 2.12.103 || APOLD1 | 2.12.107 |
apolipoprotein L domain containing 1 | 1.16275303 | 2.12.107 || CD86 | 2.12.107 | CD86 molecule |
0.874437231 | 2.12.107 | CIITA | 2.12.107 | class II, major histocompatibility complex,
transactivator | 0.408325037 | 2.12.107 || CLEC7A | 2.12.107 | C-type lectin domain family 7
member A | 0.765737967 | 2.12.107 || CNDP2 | 2.12.107 | CNDP dipeptidase 2 (metallopeptidase
M20 family) | 0.314323478 | 2.12.107 || CRTAM | 2.12.107 | cytotoxic and regulatory T-cell
molecule | 0.492695708 | 2.12.107 || CTSS | 2.12.107 | cathepsin S | 0.293650137 | 2.12.107 ||
DBN1 | 2.12.107 | drebrin 1 | 0.359697531 | 2.12.107 || DNAJB4 | 2.12.107 | DnaJ heat shock
protein family (Hsp40) member B4 | 0.328615897 | 2.12.107 || DSC2 | 2.12.107 | desmocollin 2 |
0.25527513 | 2.12.107 || GALNT6 | 2.12.107 | polypeptide N-acetylgalactosaminyltransferase 6 |
0.588788908 | 2.12.107 || GRB2 | 2.12.107 | growth factor receptor bound protein 2 | 0.331028902 |
2.12.107 || LRP5 | 2.12.107 | LDL receptor related protein 5 | 0.324201869 | 2.12.107 || MARCH1 |
2.12.107 | membrane associated ring-CH-type finger 1 | 0.664350399 | 2.12.107 || MREG | 2.12.107
| melanoregulin | 1.498263672 | 2.12.107 || MYC | 2.12.107 | v-myc avian myelocytomatosis viral
oncogene homolog | 0.346971875 | 2.12.107 || OSBPL3 | 2.12.107 | oxysterol binding protein like 3
| 0.292025729 | 2.12.107 || P2RX7 | 2.12.107 | purinergic receptor P2X 7 | 1.410038537 | 2.12.107 ||
PEG3 | 2.12.107 | paternally expressed 3 | 0.62981622 | 2.12.107 || PTCH1 | 2.12.107 | patched 1 |
0.699706032 | 2.12.107 || RAB31 | 2.12.107 | RAB31, member RAS oncogene family |
0.215426784 | 2.12.107 || SLC8A1 | 2.12.107 | solute carrier family 8 member Al | 0.382467359 |
2.12.107 || SPRY1 | 2.12.107 | sprouty RTK signaling antagonist 1 | 1.075125941 | 2.12.107 ||
ABCA7 | 2.13 | ATP binding cassette subfamily A member 7 | 0.242566393 | 2.13.114 || ABCC3
2.13 | ATP binding cassette subfamily C member 3 | 0.812744141 | - || ABCD1 | 2.13 | ATP binding
cassette subfamily D member 1 | 0.3229188 | 2.13.114 || ABHD12 | 2.13 | abhydrolase domain
containing 12 | 0.39543476 | 2.13.114 || ACP2 | 2.13 | acid phosphatase 2, lysosomal | 0.664126978 |
2.13.112 || ACP5 | 2.13 | acid phosphatase 5, tartrate resistant | 1.260127755 | 2.13.115 || ACSL4 |
2.13 | acyl-CoA synthetase long-chain family member 4 | 0.363441248 | - || ACSM5 | 2.13 | acyl-
CoA synthetase medium-chain family member 5 | 0.683890782 | 2.13.111 || ADPGK | 2.13 | ADP-
dependent glucokinase | 0.292056261 | 2.13.114 || ADRBK1 | 2.13 | adrenergic, beta, receptor
kinase 1 | 0.557067614 | 2.13.111 || AEBP2 | 2.13 | AE binding protein 2 | 0.324321466 | 2.13.111 ||
AGMAT | 2.13 | agmatinase | 0.606004319 | 2.13.111 || AGTR1 | 2.13 | angiotensin II receptor type
1 | 1.435580979 | 2.13.115 || AGTRAP | 2.13 | angiotensin II receptor associated protein |
0.31645459 | 2.13.114 || AHNAK2 | 2.13 | AHNAK nucleoprotein 2 | 1.156764235 | 2.13.109 ||
AMOTL2 | 2.13 | angiomotin like 2 | 0.690294426 | 2.13.115 || ANK2 | 2.13 | ankyrin 2, neuronal
0.853893541 | 2.13.115 || ANKIB1 | 2.13 | ankyrin repeat and IBR domain containing 1 |
0.251107709 | 2.13.111 || ANKRD13B | 2.13 | ankyrin repeat domain 13B | 0.249764536 | - ||
ANKRD44 | 2.13 | ankyrin repeat domain 44 | 0.419715692 | 2.13.117 || AP1B1 | 2.13 | adaptor
related protein complex 1 beta 1 subunit | 0.313746684 | 2.13.114 || AQP9 | 2.13 | aquaporin 9 |
3.36202372 | 2.13.109 || ARAP1 | 2.13 | ArfGAP with RhoGAP domain, ankyrin repeat and PH
domain 1 | 0.270031263 | 2.13.114 || ARFRP1 | 2.13 | ADP ribosylation factor related protein 1 |
0.314641078 | 2.13.109 || ARHGAP12 | 2.13 | Rho GTPase activating protein 12 | 0.233692783
2.13.112 || ARHGAP20 | 2.13 | Rho GTPase activating protein 20 | 3.587873186 | 2.13.116 ||
ARHGAP30 | 2.13 | Rho GTPase activating protein 30 | 0.553082233 | 2.13.117 || ARHGAP5 | 2.13
| Rho GTPase activating protein 5 | 0.262890248 | 2.13.116 || ARHGDIA | 2.13 | Rho GDP
dissociation inhibitor (GDI) alpha | 0.344731536 | 2.13.114 || ARHGEF28 | 2.13 | Rho guanine
nucleotide exchange factor 28 | 0.276366556 | 2.13.114 || ARL13B | 2.13 | ADP ribosylation factor
like GTPase 13B | 0.42198126 | 2.13.116 || ARMC7 | 2.13 | armadillo repeat containing 7 |
0.335938816 | 2.13.109 || ARPC4 | 2.13 | actin related protein 2/3 complex subunit 4 | 0.338889024
| 2.13.114 || ARRB2 | 2.13 | arrestin, beta 2 | 0.667353075 | 2.13.112 || ARSA | 2.13 | arylsulfatase A
| 0.750160235 | 2.13.111 || ATP13A1 | 2.13 | ATPase 13A1 | 0.271731484 | 2.13.109 || ATP1A1 |
2.13 | ATPase Na+/K+ transporting subunit alpha 1 | 0.256338538 | 2.13.114 || ATP2A2 | 2.13 |
ATPase sarcoplasmic/endoplasmic reticulum Ca2+ transporting 2 | 0.227494642 | 2.13.109 ||
ATP6AP1 | 2.13 | ATPase H+ transporting accessory protein 1 | 0.385387572 | 2.13.114 ||
ATP6VOB | 2.13 | ATPase H+ transporting V0 subunit b | 0.285343938 | 2.13.115 || ATP6V0C |
2.13 | ATPase H+ transporting V0 subunit c | 0.258838881 | 2.13.114 || ATP6V1B2 | 2.13 | ATPase
H+ transporting VI subunit B2 | 0.372650633 | 2.13.117 || B3GALNT1 | 2.13 | beta-1,3-N-
acetylgalactosaminyltransferase 1 (globoside blood group) | 0.780057 | 2.13.111 || BCL9L | 2.13 |
B-cell CLL/lymphoma 9-like | 0.423412072 | - || BICC1 | 2.13 | BicC family RNA binding protein 1
| 0.668961195 | 2.13.116 || BLNK | 2.13 | B-cell linker | 0.435714403 | 2.13.112 || BMF | 2.13 | Bcl2
modifying factor | 0.408173976 | 2.13.114 || BOC | 2.13 | BOC cell adhesion associated, oncogene
regulated | 0.517281217 | 2.13.116 || BRI3 | 2.13 | brain protein 13 | 0.276505057 | 2.13.114 || BTK |
2.13 | Bruton tyrosine kinase | 0.249249202 | - || BTN2A1 | 2.13 | butyrophilin subfamily 2 member
A1 | 0.298190254 | - || C10orf10 | 2.13 | chromosome 10 open reading frame 10 | 1.450562961 |
2.13.112 || C11orf74 | 2.13 | chromosome 11 open reading frame 74 | 0.356840888 | 2.13.114 ||
C11orf95 | 2.13 | chromosome 11 open reading frame 95 | 0.548649164 | 2.13.116 || C15orf40 | 2.13
| chromosome 15 open reading frame 40 | 0.352275868 | 2.13.116 || C1QA | 2.13 | complement
component 1, q subcomponent, A chain | 0.421878129 | - || C1QB | 2.13 | complement component 1,
q subcomponent, B chain | 0.572935654 | - || C1QC | 2.13 | complement component 1, q
subcomponent, C chain | 0.217455591 | - || C2orf76 | 2.13 | chromosome 2 open reading frame 76 |
0.779841415 | 2.13.117 || C3AR1 | 2.13 | complement component 3a receptor 1 | 0.844804571 | - ||
C3orf38 | 2.13 | chromosome 3 open reading frame 38 | 0.507900912 | 2.13.111 || C5AR1 | 2.13 |
complement component 5a receptor 1 | 0.611938719 | 2.13.111 || C6orf203 | 2.13 | chromosome 6
open reading frame 203 | 0.273248312 | 2.13.116 || C9orf40 | 2.13 | chromosome 9 open reading
frame 40 | 0.436229202 | 2.13.112 || CAPG | 2.13 | capping actin protein, gelsolin like |
1.250053934 | 2.13.115 || CBLB | 2.13 | Cbl proto-oncogene B, E3 ubiquitin protein ligase |
0.6990346 | 2.13.114 || CCDC34 | 2.13 | coiled-coil domain containing 34 | 0.271448568 | 2.13.109
|| CCDC71L | 2.13 | coiled-coil domain containing 71-like | 0.447881502 | 2.13.114 || CD163 | 2.13 |
CD163 molecule | 0.82788387 | - || CD33 | 2.13 | CD33 molecule | 0.458006501 | 2.13.114 || CD58 |
2.13 | CD58 molecule | 0.222572712 | 2.13.112 || CD74 | 2.13 | CD74 molecule | 0.237274973 |
2.13.112 || CD84 | 2.13 | CD84 molecule | 0.687760285 | 2.13.115 || CD99P1 | 2.13 | CD99
molecule pseudogene 1 | 0.975712846 | 2.13.114 || CDC25B | 2.13 | cell division cycle 25B |
0.234935615 | 2.13.117 || CDC42 | 2.13 | cell division cycle 42 | 0.249055075 | 2.13.114 || CDH23 |
2.13 | cadherin-related 23 | 0.235240172 | 2.13.111 || CDK2 | 2.13 | cyclin-dependent kinase 2 |
0.378352115 | 2.13.111 || CDK6 | 2.13 | cyclin-dependent kinase 6 | 0.313501183 | 2.13.114 ||
CEBPD | 2.13 | CCAAT/enhancer binding protein delta | 0.311601959 | 2.13.114 || CEP68 | 2.13 |
centrosomal protein 68kDa | 0.797869674 | 2.13.115 || CHKA | 2.13 | choline kinase alpha |
0.242642167 | 2.13.114 || CHST11 | 2.13 | carbohydrate (chondroitin 4) sulfotransferase 11 |
0.741694504 | 2.13.117 || CHSY3 | 2.13 | chondroitin sulfate synthase 3 | 0.50778913 | 2.13.114 ||
CKS1B | 2.13 | CDC28 protein kinase regulatory subunit 1B | 0.45252261 | 2.13.115 || CKS2 | 2.13 |
CDC28 protein kinase regulatory subunit 2 | 1.100558823 | 2.13.114 || CLDN11 | 2.13 | claudin 11 |
1.352899095 | 2.13.112 || CMTM8 | 2.13 | CKLF like MARVEL transmembrane domain containing
8 | 0.247960622 | - || COMT | 2.13 | catechol-O-methyltransferase | 0.240369811 | 2.13.114 ||
COTL1 | 2.13 | coactosin-like F-actin binding protein 1 | 0.419338939 | 2.13.112 || CPNE1 | 2.13 |
copine 1 | 0.306504255 | 2.13.112 || CROCC | 2.13 | ciliary rootlet coiled-coil, rootletin |
0.554760445 | 2.13.115 || CROT | 2.13 | carnitine O-octanoyltransferase | 0.275350575 | 2.13.109 ||
CSPP1 | 2.13 | centrosome and spindle pole associated protein 1 | 0.298382526 | 2.13.112 || CTBP2 |
2.13 | C-terminal binding protein 2 | 0.221798346 | 2.13.111 || CTNNAL1 | 2.13 | catenin alpha-like
1 | 0.941381818 | 2.13.114 || CTSB | 2.13 | cathepsin B | 0.444678736 | 2.13.112 || CTSD | 2.13 |
cathepsin D | 0.598024566 | 2.13.115 || CTSL | 2.13 | cathepsin L | 0.791556213 | 2.13.111 ||
DAGLB | 2.13 | diacylglycerol lipase beta | 0.412141118 | 2.13.114 || DAPK1 | 2.13 | death-
associated protein kinase 1 | 0.355467029 | 2.13.112 || DCLK1 | 2.13 | doublecortin like kinase 1 |
1.823815254 | 2.13.115 || DDAH1 | 2.13 | dimethylarginine dimethylaminohydrolase 1 |
1.054038883 | 2.13.115 || DGKA | 2.13 | diacylglycerol kinase alpha | 0.235711422 | 2.13.109 ||
DHRS7 | 2.13 | dehydrogenase/reductase (SDR family) member 7 | 0.227646413 | 2.13.114 ||
DMPK | 2.13 | dystrophia myotonica protein kinase | 0.644506824 | - || DNM2 | 2.13 | dynamin 2 |
0.307178926 | 2.13.111 || DOCK2 | 2.13 | dedicator of cytokinesis 2 | 0.396958644 | 2.13.116 ||
DOCK9 | 2.13 | dedicator of cytokinesis 9 | 0.994743112 | 2.13.115 || DOK2 | 2.13 | docking protein
2 | 0.406246015 | 2.13.112 || DOK3 | 2.13 | docking protein 3 | 1.407402243 | 2.13.117 || DTD2 |
2.13 | D-tyrosyl-tRNA deacylase 2 (putative) | 0.414817763 | 2.13.111 || DUBR | 2.13 | DPPA2
upstream binding RNA | 0.550940468 | 2.13.109 || ECT2 | 2.13 | epithelial cell transforming 2 |
0.34471067 | 2.13.114 || EFNB2 | 2.13 | ephrin-B2 | 1.356697214 | 2.13.116 || EGR1 | 2.13 | early
growth response 1 | 2.184401604 | 2.13.111 || EGR3 | 2.13 | early growth response 3 | 0.802268668 |
2.13.114 || EID2 | 2.13 | EP300 interacting inhibitor of differentiation 2 | 0.334727204 | 2.13.109 ||
ENPP4 | 2.13 | ectonucleotide pyrophosphatase/phosphodiesterase 4 (putative) | 0.459210345 |
2.13.114 || ERLIN2 | 2.13 | ER lipid raft associated 2 | 0.388337469 | 2.13.116 || ESR1 | 2.13 |
estrogen receptor 1 | 0.758299814 | 2.13.116 || ETS1 | 2.13 | ETS proto-oncogene 1, transcription
factor | 0.385794104 | 2.13.115 || EVL | 2.13 | Enah/Vasp-like | 0.51540568 | 2.13.117 || F8 | 2.13 |
coagulation factor VIII | 0.969874965 | 2.13.111 || FAM105A | 2.13 | family with sequence
similarity 105 member A | 0.379660874 | 2.13.112 || FAM109A | 2.13 | family with sequence
similarity 109 member A | 0.277290623 | 2.13.114 || FAM129B | 2.13 | family with sequence
similarity 129 member B | 0.558431272 | 2.13.114 || FAM13C | 2.13 | family with sequence
similarity 13 member C | 0.978243254 | 2.13.116 || FAM171A1 | 2.13 | family with sequence
similarity 171 member Al | 0.822847258 | 2.13.116 || FAM172A | 2.13 | family with sequence
similarity 172 member A | 0.765955214 | 2.13.111 || FAM229B | 2.13 | family with sequence
similarity 229 member B | 0.620487724 | 2.13.117 || FAM53B | 2.13 | family with sequence
similarity 53 member B | 0.220473279 | 2.13.114 || FAM78A | 2.13 | family with sequence
similarity 78 member A | 0.293370287 | 2.13.116 || FAM96A | 2.13 | family with sequence
similarity 96 member A | 0.240033343 | 2.13.112 || FAT4 | 2.13 | FAT atypical cadherin 4 |
1.070560014 | 2.13.116 || FBP1 | 2.13 | fructose-bisphosphatase 1 | 4.173658928 | 2.13.115 ||
FCER1G | 2.13 | Fc fragment of IgE receptor Ig | 0.777975804 | 2.13.112 || FCGR2B | 2.13 | Fc
fragment of IgG receptor IIb | 0.953106891 | 2.13.116 || FHL5 | 2.13 | four and a half LIM domains
5 | 1.775450295 | 2.13.112 || FKBP10 | 2.13 | FK506 binding protein 10 | 1.497576934 | 2.13.114 ||
FMNL1 | 2.13 | formin like 1 | 0.645099805 | 2.13.112 || FOXO4 | 2.13 | forkhead box O4 |
0.358715734 | 2.13.114 || FPR3 | 2.13 | formyl peptide receptor 3 | 0.974917252 | 2.13.115 || FTH1 |
2.13 | ferritin, heavy polypeptide 1 | 0.235701696 | 2.13.112 || FUS | 2.13 | FUS RNA binding
protein | 0.255595073 | - || GAA | 2.13 | glucosidase, alpha; acid | 0.86547707 | 2.13.114 ||
GALNT11 | 2.13 | polypeptide N-acetylgalactosaminyltransferase 11 | 0.248601142 | 2.13.109 ||
GBGT1 | 2.13 | globoside alpha-1,3-N-acetylgalactosaminyltransferase 1 | 0.247002466 | 2.13.115 ||
GDAP1 | 2.13 | ganglioside induced differentiation associated protein 1 | 0.357898066 | 2.13.109 ||
GFRA1 | 2.13 | GDNF family receptor alpha 1 | 1.117068893 | 2.13.116 || GHDC | 2.13 | GH3
domain containing | 0.254119438 | 2.13.112 || GIMAP7 | 2.13 | GTPase, IMAP family member 7 |
0.329129516 | 2.13.115 || GLIDR | 2.13 | glioblastoma down-regulated RNA | 1.137115929 |
2.13.109 || GM2A | 2.13 | GM2 ganglioside activator | 0.355757498 | 2.13.114 || GMPR2 | 2.13 |
guanosine monophosphate reductase 2 | 0.237416458 | - || GPNMB | 2.13 | glycoprotein nmb |
0.466321045 | 2.13.114 || GPR1 | 2.13 | G protein-coupled receptor 1 | 1.220139673 | 2.13.109 ||
GRAMD3 | 2.13 | GRAM domain containing 3 | 0.446615819 | 2.13.109 || GRIPAP1 | 2.13 | GRIP1
associated protein 1 | 0.272956233 | - || GRN | 2.13 | granulin | 0.447978794 | 2.13.114 || GSPT2 |
2.13 | Gl to S phase transition 2 | 0.359818944 | 2.13.116 || GSTO1 | 2.13 | glutathione S-transferase
omega 1 | 0.265590052 | 2.13.115 || HACD3 | 2.13 | 3-hydroxyacyl-CoA dehydratase 3 |
0.366670218 | 2.13.112 || HAPLN3 | 2.13 | hyaluronan and proteoglycan link protein 3 |
0.256133193 | - || HAVCR2 | 2.13 | hepatitis A virus cellular receptor 2 | 0.809067425 | 2.13.115 ||
HCK | 2.13 | HCK proto-oncogene, Src family tyrosine kinase | 0.591574122 | 2.13.111 ||
HDGFRP3 | 2.13 | hepatoma-derived growth factor, related protein 3 | 0.355894211 | 2.13.116 ||
HECW2 | 2.13 | HECT, C2 and WW domain containing E3 ubiquitin protein ligase 2 | 1.386960007
| 2.13.112 || HEXB | 2.13 | hexosaminidase subunit beta | 0.571878974 | 2.13.114 || HGF | 2.13 |
hepatocyte growth factor | 0.882763197 | 2.13.109 || HK3 | 2.13 | hexokinase 3 | 1.301991786 |
2.13.112 || HMG20A | 2.13 | high mobility group 20A | 0.215790117 | 2.13.116 || HMOX1 | 2.13 |
heme oxygenase 1 | 1.262125375 | 2.13.115 || HOXA10 | 2.13 | homeobox A10 | 1.078769313 |
2.13.116 || HOXA3 | 2.13 | homeobox A3 | 0.456875599 | 2.13.111 || HOXB6 | 2.13 | homeobox B6
| 0.668773405 | 2.13.114 || HOXC10 | 2.13 | homeobox C10 | 1.897134168 | 2.13.109 || HPS5 | 2.13
| HPS5, biogenesis of lysosomal organelles complex 2 subunit 2 | 0.415991705 | 2.13.116 ||
HSPA12A | 2.13 | heat shock protein family A (Hsp70) member 12A | 0.473957291 | 2.13.111 ||
HTR2A | 2.13 | 5-hydroxytryptamine receptor 2A | 0.430489177 | 2.13.111 || HVCN1 | 2.13 |
hydrogen voltage gated channel 1 | 0.395585015 | 2.13.114 || IDH2 | 2.13 | isocitrate dehydrogenase
2 (NADP+), mitochondrial | 0.630488179 | 2.13.111 || IGF2BP2 | 2.13 | insulin like growth factor 2
mRNA binding protein 2 | 0.954990871 | 2.13.111 || IGIP | 2.13 | IgA-inducing protein |
0.701594348 | 2.13.116 || IGSF10 | 2.13 | immunoglobulin superfamily member 10 | 0.878588832 |
2.13.111 || IKZF4 | 2.13 | IKAROS family zinc finger 4 | 0.325366992 | 2.13.109 || IL13RA2 | 2.13 |
interleukin 13 receptor subunit alpha 2 | 2.550295806 | 2.13.112 || IL15 | 2.13 | interleukin 15 |
0.814306198 | 2.13.117 || IL17RA | 2.13 | interleukin 17 receptor A | 0.455702783 | 2.13.117 ||
INO80D | 2.13 | INO80 complex subunit D | 0.971483252 | 2.13.111 || INPP4B | 2.13 | inositol
polyphosphate-4-phosphatase type II B | 1.917873318 | 2. 13.112 || IQCK | 2.13 | IQ motif
containing K | 0.322032485 | 2.13.115 || IRF8 | 2.13 | interferon regulatory factor 8 | 0.516252625 |
2.13.117 || ITGA6 | 2.13 | integrin subunit alpha 6 | 0.400699663 | 2.13.115 || ITGB3 | 2.13 | integrin
subunit beta 3 | 1.493493017 | 2.13.115 || KBTBD7 | 2.13 | kelch repeat and BTB domain
containing 7 | 0.6206987 | 2.13.111 || KCNN4 | 2.13 | potassium calcium-activated channel
subfamily N member 4 | 1.262571821 | 2.13.115 || KCTD5 | 2.13 | potassium channel
tetramerization domain containing 5 | 0.459915482 | 2.13.109 || KHDRBS3 | 2.13 | KH domain
containing, RNA binding, signal transduction associated 3 | 1.029569179 | 2.13.115 || KIAA1468 |
2.13 | KIAA1468 | 0.253685207 | 2.13.117 || KIAA1671 | 2.13 | KIAA1671 | 0.844416438 |
2.13.116 || KIFIC | 2.13 | kinesin family member 1C | 0.499234725 | 2.13.111 || KLF3-AS1 | 2.13 |
KLF3 antisense RNA 1 | 2.13451177 | 2.13.116 || KLF9 | 2.13 | Kruppel-like factor 9 | 0.553886869
| 2.13.114 || KLHDC1 | 2.13 | kelch domain containing 1 | 0.353289329 | 2.13.109 || LAPTM5 | 2.13
| lysosomal protein transmembrane 5 | 0.463046131 | 2.13.112 || LAT2 | 2.13 | linker for activation
of T-cells family member 2 | 0.574086734 | 2.13.111 || LDLRAD4 | 2.13 | low density lipoprotein
receptor class A domain containing 4 | 0.671584873 | 2.13.109 || LHFP | 2.13 | lipoma HMGIC
fusion partner | 0.625315112 | 2.13.115 || LIFR | 2.13 | leukemia inhibitory factor receptor alpha |
2.429454608 | 2.13.112 || LINC00304 | 2.13 | long intergenic non-protein coding RNA 304 |
0.393501478 | 2.13.114 || LINC00632 | 2.13 | long intergenic non-protein coding RNA 632 |
1.047050423 | 2.13.109 || LINC00936 | 2.13 | long intergenic non-protein coding RNA 936 |
0.700387452 | 2.13.109 || LPAR4 | 2.13 | lysophosphatidic acid receptor 4 | 0.218232239 | 2.13.116
|| LRIF1 | 2.13 | ligand dependent nuclear receptor interacting factor 1 | 0.503312043 | 2.13.117 ||
LRIG3 | 2.13 | leucine-rich repeats and immunoglobulin like domains 3 | 0.499218829 | 2.13.116 ||
LRRC16A | 2.13 | leucine rich repeat containing 16A | 0.908080901 | 2.13.112 || LRRC4C | 2.13 |
leucine rich repeat containing 4C | 0.69611547 | 2.13.116 || LTA4H | 2.13 | leukotriene A4
hydrolase | 0.72641255 | 2.13.112 || LYST | 2.13 | lysosomal trafficking regulator | 0.651096213 | - ||
MAD2L1 | 2.13 | MAD2 mitotic arrest deficient-like 1 (yeast) | 1.100419452 | 2.13.114 || MAGI2 |
2.13 | membrane associated guanylate kinase, WW and PDZ domain containing 2 | 1.075266587 |
2.13.116 || MAL | 2.13 | mal T-cell differentiation protein | 0.394359158 | 2.13.109 || MAN2B1 |
2.13 | mannosidase alpha class 2B member 1 | 0.467641762 | 2.13.112 || MAPIB | 2.13 |
microtubule associated protein 1B | 1.701090816 | 2.13.112 || MAP1S | 2.13 | microtubule
associated protein 1S | 0.566819557 | - || MAPKAP1 | 2.13 | mitogen-activated protein kinase
associated protein 1 | 0.334436347 | 2.13.111 || MARCO | 2.13 | macrophage receptor with
collagenous structure | 2.002377443 | 2.13.115 || MATN2 | 2.13 | matrilin 2 | 2.369189186 |
2.13.115 || MBOAT1 | 2.13 | membrane bound O-acyltransferase domain containing 1 |
0.357216125 | 2.13.114 || MCC | 2.13 | mutated in colorectal cancers | 1.2531783 | 2.13.116 ||
MCTP1 | 2.13 | multiple C2 and transmembrane domain containing 1 | 0.988495346 | 2.13.114 ||
ME1 | 2.13 | malic enzyme 1, NADP(+)-dependent, cytosolic | 0.278326942 | 2.13.114 || MEF2A |
2.13 | myocyte enhancer factor 2A | 0.350986547 | 2.13.117 || METTL18 | 2.13 | methyltransferase
like 18 | 0.601436544 | 2.13.115 || MFSD1 | 2.13 | major facilitator superfamily domain containing 1
| 0.240993841 | 2.13.112 || MGAT1 | 2.13 | mannosyl (alpha-1,3-)-glycoprotein beta-1,2-N-
acetylglucosaminyltransferase | 0.350451991 | 2.13.114 ?| MGME1 | 2.13 | mitochondrial genome
maintenance exonuclease 1 | 0.220814438 | 2.13.117 || MICU3 | 2.13 | mitochondrial calcium
uptake family member 3 | 1.463454331 | 2.13.116 || MINA | 2.13 | MYC induced nuclear antigen |
0.283548651 | 2.13.114 || MIR99AHG | 2.13 | mir-99a-let-7c cluster host gene | 4.859450805 |
2.13.111 || MKL2 | 2.13 | MKL/myocardin-like 2 | 0.633333227 | 2.13.115 | MOV10 | 2.13 | Mov10
RISC complex RNA helicase | 0.377095973 | - || MPZL2 | 2.13 | myelin protein zero like 2 |
0.894110176 | 2.13.111 || MRPS30 | 2.13 | mitochondrial ribosomal protein S30 | 0.249330581 |
2.13.111 || MRVI1 | 2.13 | murine retrovirus integration site 1 homolog | 0.233691875 | 2.13.115 ||
MS4A14 | 2.13 | membrane spanning 4-domains A14 | 0.900422952 | 2.13.114 || MS4A2 | 2.13 |
membrane spanning 4-domains A2 | 1.073406565 | 2.13.116 || MS4A4A | 2.13 | membrane
spanning 4-domains A4A | 0.516479686 | - || MS4A6A | 2.13 | membrane spanning 4-domains A6A
| 0.363578436 | 2.13.111 || MSH2 | 2.13 | mutS homolog 2 | 0.505425044 | 2.13.112 || MSR1 | 2.13 |
macrophage scavenger receptor 1 | 0.903071728 | - || MTSS1 | 2.13 | metastasis suppressor 1 |
0.257173936 | - || MVP | 2.13 | major vault protein | 0.242014911 | 2.13.114 || MYRIP | 2.13 |
myosin VIIA and Rab interacting protein | 2.974821829 | 2.13.111 || NAGA | 2.13 | N-
acetylgalactosaminidase, alpha- | 0.418428557 | 2.13.112 || NAGPA | 2.13 | N-acetylglucosamine-1-
phosphodiester alpha-N-acetylglucosaminidase | 0.325417838 | 2.13.114 || NAPIL3 | 2.13 |
nucleosome assembly protein 1 like 3 | 1.765809462 | 2.13.111 || NAV1 | 2.13 | neuron navigator 1 |
0.606213548 | 2.13.109 || NAV2 | 2.13 | neuron navigator 2 | 0.735707401 | 2.13.109 || NCEH1 |
2.13 | neutral cholesterol ester hydrolase 1 | 0.642700746 | - || NEDD4L | 2.13 | neural precursor cell
expressed, developmentally down-regulated 4-like, E3 ubiquitin protein ligase | 0.812970685 |
2.13.111 || NEGR1 | 2.13 | neuronal growth regulator 1 | 4.682478671 | 2.13.111 || NET1 | 2.13 |
neuroepithelial cell transforming 1 | 0.867597081 | 2.13.112 | NEURL2 | 2.13 | neuralized E3
ubiquitin protein ligase 2 | 0.990915265 | - || NFAM1 | 2.13 | NFAT activating protein with ITAM
motif 1 | 0.393442758 | 2.13.116 || NFE2L3 | 2.13 | nuclear factor, erythroid 2 like 3 | 1.400239678 |
2.13.116 || NFIA | 2.13 | nuclear factor I/A | 1.269390224 | 2.13.111 || NFIB | 2.13 | nuclear factor
I/B | 1.594047921 | 2.13.111 || NINL | 2.13 | ninein like | 0.38619854 | 2.13.116 || NOVA1 | 2.13 |
neuro-oncological ventral antigen 1 | 2.94232798 | 2.13.111 || NPC1 | 2.13 | Niemann-Pick disease,
type C1 | 0.659163398 | 2.13.117 || NR1H3 | 2.13 | nuclear receptor subfamily 1 group H member 3
| 0.772364085 | 2.13.112 || NR5A2 | 2.13 | nuclear receptor subfamily 5 group A member 2 |
0.259931 | - || NSUN6 | 2.13 | NOP2/Sun RNA methyltransferase family member 6 | 0.682024825
2.13.116 || NTRK2 | 2.13 | neurotrophic tyrosine kinase, receptor, type 2 | 4.633944435 | 2.13.111 ||
NUP62 | 2.13 | nucleoporin 62kDa | 0.385630702 | 2.13.117 || ODF2L | 2.13 | outer dense fiber of
sperm tails 2 like | 0.289734932 | 2.13.112 || OMA1 | 2.13 | OMA1 zinc metallopeptidase |
0.277222184 | 2.13.111 || OSBPL10 | 2.13 | oxysterol binding protein like 10 | 0.502131041 |
2.13.115 || OSGEPL1 | 2.13 | O-sialoglycoprotein endopeptidase-like 1 | 0.601682073 | 2.13.112 ||
OSGIN2 | 2.13 | oxidative stress induced growth inhibitor family member 2 | 0.485072903 |
2.13.117 || P2RX4 | 2.13 | purinergic receptor P2X 4 | 0.778628666 | 2.13.112 || P4HB | 2.13 | prolyl
4-hydroxylase subunit beta | 0.342471626 | 2.13.114 || PARP10 | 2.13 | poly(ADP-ribose)
polymerase family member 10 | 0.543773678 | 2.13.111 || PARP3 | 2.13 | poly(ADP-ribose)
polymerase family member 3 | 0.264796188 | 2.13.112 || PARP6 | 2.13 | poly(ADP-ribose)
polymerase family member 6 | 0.25692068 | 2.13.114 || PARVB | 2.13 | parvin beta | 0.70562519 |
2.13.114 || PBX1 | 2.13 | pre-B-cell leukemia homeobox 1 | 0.688052051 | 2.13.111 || PCDH18 |
2.13 | protocadherin 18 | 1.056745674 | 2.13.115 || PCDH9 | 2.13 | protocadherin 9 | 1.739731223 |
2.13.116 || PCDHB16 | 2.13 | protocadherin beta 16 | 2.67915659 | 2.13.109 || PCK2 | 2.13 |
phosphoenolpyruvate carboxykinase 2, mitochondrial | 0.278442824 | 2.13.115 || PCSK5 | 2.13 |
proprotein convertase subtilisin/kexin type 5 | 0.734443093 | 2.13.111 || PCYOXIL | 2.13 |
prenylcysteine oxidase 1 like | 0.284605579 | 2.13.111 || PDAP1 | 2.13 | PDGFA associated protein
1 | 0.402836026 | 2.13.114 || PDIA4 | 2.13 | protein disulfide isomerase family A member 4 |
0.215029764 | - || PDK4 | 2.13 | pyruvate dehydrogenase kinase 4 | 5.958649538 | - || PEX12 | 2.13 |
peroxisomal biogenesis factor 12 | 0.563432071 | 2.13.112 || PIK3IP1 | 2.13 | phosphoinositide-3-
kinase interacting protein 1 | 0.330308467 | 2.13.114 || PIK3R5 | 2.13 | phosphoinositide-3-kinase
regulatory subunit 5 | 0.676437862 | 2.13.114 || PILRA | 2.13 | paired immunoglobin-like type 2
receptor alpha | 0.994717293 | 2.13.112 || PKM | 2.13 | pyruvate kinase, muscle | 0.485963464 |
2.13.114 || PKN2 | 2.13 | protein kinase N2 | 0.366914792 | 2.13.114 || PLA2G15 | 2.13 |
phospholipase A2 group XV | 0.308512115 | 2.13.115 || PLAUR | 2.13 | plasminogen activator,
urokinase receptor | 1.130963658 | - || PLBD1 | 2.13 | phospholipase B domain containing 1 |
0.798153641 | 2.13.114 || PLCB1 | 2.13 | phospholipase C beta 1 | 1.300908665 | 2.13.116 || PLD3 |
2.13 | phospholipase D family member 3 | 0.447032144 | 2.13.114 || PLEKHH2 | 2.13 | pleckstrin
homology, MyTH4 and FERM domain containing H2 | 1.691812098 | 2.13.115 || PLP2 | 2.13 |
proteolipid protein 2 (colonic epithelium-enriched) | 0.241328907 | 2.13.114 || PLSCR4 | 2.13 |
phospholipid scramblase 4 | 0.947501888 | 2.13.111 || PLTP | 2.13 | phospholipid transfer protein |
0.303211382 | - || POLR3K | 2.13 | polymerase (RNA) III subunit K | 0.233338628 | 2.13.114 ||
PON2 | 2.13 | paraoxonase 2 | 0.237306537 | 2.13.114 || PPDPF | 2.13 | pancreatic progenitor cell
differentiation and proliferation factor | 0.310586024 | 2.13.114 || PPL | 2.13 | periplakin |
2.509024142 | 2.13.111 || PPP1R2 | 2.13 | protein phosphatase 1 regulatory inhibitor subunit 2 |
0.219633925 | 2.13.109 || PREX1 | 2.13 | phosphatidylinositol-3,4,5-trisphosphate-dependent Rac
exchange factor 1 | 0.250217617 | 2.13.117 || PRICKLE1 | 2.13 | prickle planar cell polarity protein
1 | 0.522802685 | 2.13.111 || PRRG1 | 2.13 | proline rich Gla (G-carboxyglutamic acid) 1 |
0.653723801 | 2.13.114 || PRRT2 | 2.13 | proline rich transmembrane protein 2 | 0.234783992 |
2.13.116 || PRTFDC1 | 2.13 | phosphoribosyl transferase domain containing 1 | 1.210007463 |
2.13.116 || PTAFR | 2.13 | platelet activating factor receptor | 0.478181238 | 2.13.114 || PTGER2 |
2.13 | prostaglandin E receptor 2 | 0.628419606 | 2.13.112 || PTPN13 | 2.13 | protein tyrosine
phosphatase, non-receptor type 13 | 0.881444917 | 2.13.112 || PTPRB | 2.13 | protein tyrosine
phosphatase, receptor type B | 1.346443092 | 2.13.115 || PTPRJ | 2.13 | protein tyrosine
phosphatase, receptor type J | 0.758561814 | 2.13.117 || PTPRS | 2.13 | protein tyrosine phosphatase,
receptor type S | 1.499861491 | 2.13.109 || PUS7 | 2.13 | pseudouridylate synthase 7 (putative) |
0.302191589 | 2.13.112 || PVR | 2.13 | poliovirus receptor | 0.219811576 | 2.13.116 || QRSL1 | 2.13 |
glutaminyl-tRNA synthase (glutamine-hydrolyzing)-like 1 | 0.410479951 | 2.13.115 || QSOX1 | 2.13
| quiescin sulfhydryl oxidase 1 | 0.368927339 | 2. 13.114 || RAB30 | 2.13 | RAB30, member RAS
oncogene family | 1.077446891 | 2.13.112 || RAPGEF1 | 2.13 | Rap guanine nucleotide exchange
factor 1 | 0.572073564 | 2.13.117 || RBM47 | 2.13 | RNA binding motif protein 47 | 0.486946946 |
2.13.117 || RBMS3 | 2.13 | RNA binding motif, single stranded interacting protein 3 | 2.229894728 |
2.13.111 || RCAN2 | 2.13 | regulator of calcineurin 2 | 1.848732911 | 2.13.116 || RFX7 | 2.13 |
regulatory factor X7 | 0.222841625 | 2.13.111 || RFXAP | 2.13 | regulatory factor X associated
protein | 0.316740682 | 2.13.116 || RGCC | 2.13 | regulator of cell cycle | 0.714836677 | 2.13.112 ||
RGS14 | 2.13 | regulator of G-protein signaling 14 | 0.262440781 | 2.13.111 || RGS19 | 2.13 |
regulator of G-protein signaling 19 | 0.373091892 | 2.13.111 || RHOBTB3 | 2.13 | Rho related BTB
domain containing 3 | 0.984989625 | - || RHOQ | 2.13 | ras homolog family member Q |
0.241573272 | 2.13.114 || RMND1 | 2.13 | required for meiotic nuclear division 1 homolog |
0.27153658 | 2.13.109 || RNF130 | 2.13 | ring finger protein 130 | 0.3114708 | 2.13.114 || RNF149 |
2.13 | ring finger protein 149 | 0.242885372 | 2.13.109 || RNF166 | 2.13 | ring finger protein 166 |
0.238032034 | 2.13.112 || RNF19B | 2.13 | ring finger protein 19B | 0.527534608 | - || RNF220 | 2.13
| ring finger protein 220 | 0.446547478 | - || RNPC3 | 2.13 | RNA binding region (RNP1, RRM)
containing 3 | 0.431345942 | 2.13.111 || RNPEP | 2.13 | arginyl aminopeptidase | 0.214773118 |
2.13.112 || RTN1 | 2.13 | reticulon 1 | 1.017538562 | 2.13.111 || RUNX3 | 2.13 | runt related
transcription factor 3 | 1.275263144 | 2.13.117 || S100A9 | 2.13 | S100 calcium binding protein A9 |
1.27256495 | 2.13.109 || SCRN3 | 2.13 | secernin 3 | 0.248950373 | 2.13.114 || SDC3 | 2.13 |
syndecan 3 | 0.223505318 | 2.13.117 || SEC22B | 2.13 | SEC22 homolog B, vesicle trafficking
protein (gene/pseudogene) | 0.2457394 | 2.13.114 || SEMA3E | 2.13 | semaphorin 3E | 3.704350922
| 2.13.109 | SEMA6A | 2.13 | semaphorin 6A | 1.425697763 | 2.13.112 || SENP7 | 2.13 |
SUMO1/sentrin specific peptidase 7 | 0.340935043 | 2.13.112 || SEPT6 | 2.13 | septin 6 |
0.311496997 | 2.13.117 || SESN1 | 2.13 | sestrin 1 | 0.400746385 | - || SETBP1 | 2.13 | SET binding
protein 1 | 1.268880786 | 2.13.109 || SETD9 | 2.13 | SET domain containing 9 | 0.43506454 |
2.13.112 || SFRP2 | 2.13 | secreted frizzled-related protein 2 | 4.809938046 | 2.13.117 || SGOL2 |
2.13 | shugoshin-like 2 (S. pombe) | 0.677440331 | 2.13.114 || SH3BGRL3 | 2.13 | SH3 domain
binding glutamate rich protein like 3 | 0.332563528 | 2.13.114 || SH3TC1 | 2.13 | SH3 domain and
tetratricopeptide repeats 1 | 0.63890962 | - || SHTN1 | 2.13 | shootin 1 | 0.220706261 | - || SIAH2 |
2.13 | siah E3 ubiquitin protein ligase 2 | 0.273356021 | 2.13.109 || SIGLEC1 | 2.13 | sialic acid
binding Ig like lectin 1 | 0.544833518 | 2.13.114 || SIRPA | 2.13 | signal regulatory protein alpha |
0.518829285 | 2.13.114 || SLC11A2 | 2.13 | solute carrier family 11 member 2 | 0.215419556 |
2.13.115 || SLC15A3 | 2.13 | solute carrier family 15 member 3 | 0.867682008 | 2.13.112 ||
SLC16A7 | 2.13 | solute carrier family 16 member 7 | 1.623659067 | 2.13.114 || SLC25A19 | 2.13 |
solute carrier family 25 member 19 | 1.556127813 | 2.13.112 | SLC25A37 | 2.13 | solute carrier
family 25 member 37 | 0.814925352 | 2.13.111 || SLC25A45 | 2.13 | solute carrier family 25
member 45 | 0.265283041 | 2.13.111 || SLC29A3 | 2.13 | solute carrier family 29 member 3 |
0.289963854 | - || SLC36A1 | 2.13 | solute carrier family 36 member 1 | 0.763146391 | 2.13.109 ||
SLC37A2 | 2.13 | solute carrier family 37 member 2 | 0.490969374 | 2.13.115 || SLC44A2 | 2.13 |
solute carrier family 44 member 2 | 0.405020492 | 2.13.115 || SLC4A4 | 2.13 | solute carrier family
4 member 4 | 0.349407919 | 2.13.111 || SLC7A7 | 2.13 | solute carrier family 7 member 7 |
0.812936831 | 2.13.115 || SLC9A6 | 2.13 | solute carrier family 9 member A6 | 0.221078859 |
2.13.114 || SLCO2B1 | 2.13 | solute carrier organic anion transporter family member 2B1 |
0.464845675 | 2.13.112 || SMAD6 | 2.13 | SMAD family member 6 | 0.547174726 | 2.13.116 ||
SMAD9 | 2.13 | SMAD family member 9 | 2.670235747 | 2.13.116 || SMPDL3A | 2.13 |
sphingomyelin phosphodiesterase acid like 3A | 0.31952755 | 2.13.114 || SNX16 | 2.13 | sorting
nexin 16 | 0.407255662 | 2.13.115 || SOCS5 | 2.13 | suppressor of cytokine signaling 5 |
0.274385882 | 2.13.111 || SOWAHC | 2.13 | sosondowah ankyrin repeat domain family member C |
0.471996181 | 2.13.111 || SP140L | 2.13 | SP140 nuclear body protein like | 0.290145129 | 2.13.117
| SPI1 | 2.13 | Spi-1 proto-oncogene | 1.01704837 | 2.13.112 || SPPL2A | 2.13 | signal peptide
peptidase like 2A | 0.293854081 | 2.13.111 || SRPX | 2.13 | sushi repeat containing protein, X-linked
| 1.127971398 | 2.13.115 || SSPN | 2.13 | sarcospan | 0.631170689 | 2.13.109 || STARD8 | 2.13 |
StAR related lipid transfer domain containing 8 | 0.362554692 | 2.13.109 || STK3 | 2.13 |
serine/threonine kinase 3 | 0.325402616 | 2.13.111 || STON1 | 2.13 | stonin 1 | 1.356403913 |
2.13.111 | SUSD2 | 2.13 | sushi domain containing 2 | 1.705192024 | 2.13.115 || SYNJ2 | 2.13 |
synaptojanin 2 | 1.185253446 | 2.13.111 || SYNPO2 | 2.13 | synaptopodin 2 | 3.221199695 | 2.13.112
|| TAB1 | 2.13 | TGF-beta activated kinase 1/MAP3K7 binding protein 1 | 0.241853879 | 2.13.112 ||
TBXAS1 | 2.13 | thromboxane A synthase 1 | 0.708020188 | 2.13.115 || TCF7L2 | 2.13 |
transcription factor 7 like 2 | 0.638065185 | 2.13.115 || TCFL5 | 2.13 | transcription factor-like 5
(basic helix-loop-helix) | 0.516830521 | 2.13.116 || TFEC | 2.13 | transcription factor EC |
1.194345762 | 2.13.112 || THAP8 | 2.13 | THAP domain containing 8 | 0.216626417 | 2.13.114 ||
THRB | 2.13 | thyroid hormone receptor beta | 0.935485167 | 2.13.116 || TM6SF1 | 2.13 |
transmembrane 6 superfamily member 1 | 0.240612663 | 2.13.112 || TMEM129 | 2.13 |
transmembrane protein 129 | 0.263432145 | 2.13.114 || TMEM133 | 2.13 | transmembrane protein
133 | 1.017890961 | 2.13.115 || TMEM176A | 2.13 | transmembrane protein 176A | 0.282487188 |
2.13.114 || TMEM176B | 2.13 | transmembrane protein 176B | 0.316009486 | 2.13.114 ||
TMEM198B | 2.13 | transmembrane protein 198B (pseudogene) | 0.333031752 | 2.13.114 ||
TMEM259 | 2.13 | transmembrane protein 259 | 0.455797342 | 2.13.114 || TMEM64 | 2.13 |
transmembrane protein 64 | 0.598506919 | 2.13.114 || TMEM70 | 2.13 | transmembrane protein 70 |
0.342005197 | 2.13.109 || TMTC2 | 2.13 | transmembrane and tetratricopeptide repeat containing 2 |
0.978419575 | 2.13.114 || TNFAIP2 | 2.13 | TNF alpha induced protein 2 | 0.51361715 | 2.13.112 ||
TNFSF13 | 2.13 | tumor necrosis factor superfamily member 13 | 0.45299741 | 2.13.112 || TNK2 |
2.13 | tyrosine kinase, non-receptor, 2 | 0.227917443 | 2.13.114 || TNS3 | 2.13 | tensin 3 |
0.31387926 | 2.13.117 || TPP1 | 2.13 | tripeptidyl peptidase I | 0.279185597 | 2.13.114 || TREM2 |
2.13 | triggering receptor expressed on myeloid cells 2 | 2.389008842 | 2.13.115 || TRHDE-AS1 |
2.13 | TRHDE antisense RNA 1 | 0.962822387 | 2.13.111 || TRIM2 | 2.13 | tripartite motif
containing 2 | 2.395669817 | 2.13.111 || TRMT6 | 2.13 | tRNA methyltransferase 6 | 0.674126391 |
2.13.114 || TRPC1 | 2.13 | transient receptor potential cation channel subfamily C member 1 |
0.787398783 | 2.13.116 || TSC22D3 | 2.13 | TSC22 domain family member 3 | 0.831241773 |
2.13.114 || TSC22D4 | 2.13 | TSC22 domain family member 4 | 0.32029484 | 2.13.109 || TSHZ2 |
2.13 | teashirt zinc finger homeobox 2 | 1.052241312 | 2.13.111 || TSHZ3 | 2.13 | teashirt zinc finger
homeobox 3 | 0.887191214 | 2.13.111 || TSPAN4 | 2.13 | tetraspanin 4 | 0.248755326 | 2.13.114 ||
TSPAN7 | 2.13 | tetraspanin 7 | 2.212393459 | 2.13.115 || TSPO | 2.13 | translocator protein |
0.237547491 | 2.13.114 || TSPYL4 | 2.13 | TSPY-like 4 | 0.279529651 | 2.13.116 || TTC38 | 2.13 |
tetratricopeptide repeat domain 38 | 0.297274267 | 2.13.114 || TTC7A | 2.13 | tetratricopeptide
repeat domain 7A | 0.370564594 | 2.13.114 || TUBB2A | 2.13 | tubulin beta 2A class IIa |
0.325023776 | 2.13.117 || TULP3 | 2.13 | tubby like protein 3 | 0.97655164 | 2.13.111 || TYROBP |
2.13 | TYRO protein tyrosine kinase binding protein | 0.395515118 | 2.13.112 || UBE2D1 | 2.13 |
ubiquitin conjugating enzyme E2D 1 | 0.308882488 | 2.13.114 || UBXN11 | 2.13 | UBX domain
protein 11 | 0.290569158 | 2.13.115 || UNC93B1 | 2.13 | unc-93 homolog B1 (C. elegans) |
0.897472824 | 2.13.112 || USB1 | 2.13 | U6 snRNA biogenesis 1 | 0.252964287 | 2.13.111 || USP30 |
2.13 | ubiquitin specific peptidase 30 | 0.230007773 | 2.13.116 || USP31 | 2.13 | ubiquitin specific
peptidase 31 | 0.873679346 | 2.13.115 || UST | 2.13 | uronyl-2-sulfotransferase | 1.849184976 |
2.13.109 || VAMP8 | 2.13 | vesicle associated membrane protein 8 | 0.447244737 | 2.13.112 || VAV1
| 2.13 | vav guanine nucleotide exchange factor 1 | 0.540446216 | - || VDAC1 | 2.13 | voltage
dependent anion channel 1 | 0.256143418 | 2.13.115 || VPS9D1 | 2.13 | VPS9 domain containing 1 |
0.442328486 | 2.13.112 || VSIG4 | 2.13 | V-set and immunoglobulin domain containing 4 |
0.824504065 | - || WASF3 | 2.13 | WAS protein family member 3 | 1.618709402 | 2.13.111 ||
WDFY4 | 2.13 | WDFY family member 4 | 0.310390837 | 2.13.117 || WIPI1 | 2.13 | WD repeat
domain, phosphoinositide interacting 1 | 0.308108308 | 2.13.111 || WRAP73 | 2.13 | WD repeat
containing, antisense to TP73 | 0.273668604 | 2.13.112 || WWOX | 2.13 | WW domain containing
oxidoreductase | 0.689727208 | 2.13.116 || WWTR1 | 2.13 | WW domain containing transcription
regulator 1 | 0.729080311 | 2.13.116 || XG | 2.13 | Xg blood group | 1.753857649 | 2.13.111 ||
YWHAH | 2.13 | tyrosine 3-monooxygenase/tryptophan 5-monooxygenase activation protein eta |
0.33826808 | 2.13.112 || ZBTB10 | 2.13 | zinc finger and BTB domain containing 10 | 0.855741505 |
2.13.111 || ZBTB16 | 2.13 | zinc finger and BTB domain containing 16 | 3.507241815 | 2.13.114 ||
ZBTB20 | 2.13 | zinc finger and BTB domain containing 20 | 0.518239496 | 2.13.109 || ZC2HCIA |
2.13 | zinc finger C2HC-type containing 1A | 0.324258645 | 2.13.112 || ZC3H12D | 2.13 | zinc
finger CCCH-type containing 12D | 0.684374131 | 2.13.117 || ZDHHC7 | 2.13 | zinc finger DHHC-
type containing 7 | 0.267367989 | 2.13.114 || ZMYND15 | 2.13 | zinc finger MYND-type containing
15 | 0.466281515 | 2.13.114 || ZNF138 | 2.13 | zinc finger protein 138 | 0.308645533 | 2.13.114 ||
ZNF260 | 2.13 | zinc finger protein 260 | 0.224175965 | 2.13.112 || ZNF496 | 2.13 | zinc finger
protein 496 | 0.220785982 | 2.13.116 || ZNF521 | 2.13 | zinc finger protein 521 | 1.320710484 |
2.13.116 || ZNF542P | 2.13 | zinc finger protein 542, pseudogene | 0.215619471 | 2.13.109 ||
ZNF559 | 2.13 | zinc finger protein 559 | 0.388113122 | 2.13.116 || ZNF652 | 2.13 | zinc finger
protein 652 | 1.650701242 | - || ZNF677 | 2.13 | zinc finger protein 677 | 0.652095407 | 2.13.116 ||
ZNF70 | 2.13 | zinc finger protein 70 | 0.68226777 | 2.13.109 || ZNF844 | 2.13 | zinc finger protein
844 | 0.521160528 | 2.13.109 || ZNF880 | 2.13 | zinc finger protein 880 | 0.77964843 | 2.13.109 ||
ZSCAN26 | 2.13 | zinc finger and SCAN domain containing 26 | 0.35784647 | 2.13.116 || AHNAK2
| 2.13.109 | AHNAK nucleoprotein 2 | 1.156764235 | 2.13.109 || AQP9 | 2.13.109 | aquaporin 9 |
3.36202372 | 2.13.109 || ARFRP1 | 2.13.109 | ADP ribosylation factor related protein 1 |
0.314641078 | 2.13.109 || ARMC7 | 2.13.109 | armadillo repeat containing 7 | 0.335938816 |
2.13.109 || ATP13A1 | 2.13.109 | ATPase 13A1 | 0.271731484 | 2.13.109 | ATP2A2 | 2.13.109 ||
ATPase sarcoplasmic/endoplasmic reticulum Ca2+ transporting 2 | 0.227494642 | 2.13.109 ||
CCDC34 | 2.13.109 | coiled-coil domain containing 34 | 0.271448568 | 2.13.109 || CROT | 2.13.109
| carnitine O-octanoyltransferase | 0.275350575 | 2.13.109 || DGKA | 2.13.109 | diacylglycerol
kinase alpha | 0.235711422 | 2.13.109 || DUBR | 2.13.109 | DPPA2 upstream binding RNA |
0.550940468 | 2.13.109 || EID2 | 2.13.109 | EP300 interacting inhibitor of differentiation 2 |
0.334727204 | 2.13.109 || GALNT11 | 2.13.109 | polypeptide N-acetylgalactosaminyltransferase 11 |
0.248601142 | 2.13.109 || GDAP1 | 2.13.109 | ganglioside induced differentiation associated protein
1 | 0.357898066 | 2.13.109 || GLIDR | 2.13.109 | glioblastoma down-regulated RNA | 1.137115929 |
2.13.109 || GPR1 | 2.13.109 | G protein-coupled receptor 1 | 1.220139673 | 2.13.109 || GRAMD3 |
2.13.109 | GRAM domain containing 3 | 0.446615819 | 2.13.109 || HGF | 2.13.109 | hepatocyte
growth factor | 0.882763197 | 2.13.109 || HOXC10 | 2.13.109 | homeobox C10 | 1.897134168 |
2.13.109 || IKZF4 | 2.13.109 | IKAROS family zinc finger 4 | 0.325366992 | 2.13.109 || KCTD5 |
2.13.109 | potassium channel tetramerization domain containing 5 | 0.459915482 | 2.13.109 ||
KLHDC1 | 2.13.109 | kelch domain containing 1 | 0.353289329 | 2.13.109 || LDLRAD4 | 2.13.109 |
low density lipoprotein receptor class A domain containing 4 | 0.671584873 | 2.13.109 ||
LINC00632 | 2.13.109 | long intergenic non-protein coding RNA 632 | 1.047050423 | 2.13.109 ||
LINC00936 | 2.13.109 | long intergenic non-protein coding RNA 936 | 0.700387452 | 2.13.109 ||
MAL | 2.13.109 | mal T-cell differentiation protein | 0.394359158 | 2.13.109 || NAV1 | 2.13.109 |
neuron navigator 1 | 0.606213548 | 2.13.109 || NAV2 | 2.13.109 | neuron navigator 2 | 0.735707401 |
2.13.109 || PCDHB16 | 2.13.109 | protocadherin beta 16 | 2.67915659 | 2.13.109 || PPP1R2 |
2.13.109 | protein phosphatase 1 regulatory inhibitor subunit 2 | 0.219633925 | 2.13.109 || PTPRS |
2.13.109 | protein tyrosine phosphatase, receptor type S | 1.499861491 | 2.13.109 || RMND1 |
2.13.109 | required for meiotic nuclear division 1 homolog | 0.27153658 | 2.13.109 || RNF149 |
2.13.109 | ring finger protein 149 | 0.242885372 | 2.13.109 || S100A9 | 2.13.109 | S100 calcium
binding protein A9 | 1.27256495 | 2.13.109 || SEMA3E | 2.13.109 | semaphorin 3E | 3.704350922 |
2.13.109 || SETBP1 | 2.13.109 | SET binding protein 1 | 1.268880786 | 2.13.109 || SIAH2 | 2.13.109
| siah E3 ubiquitin protein ligase 2 | 0.273356021 | 2.13.109 || SLC36A1 | 2.13.109 | solute carrier
family 36 member 1 | 0.763146391 | 2.13.109 || SSPN | 2.13.109 | sarcospan | 0.631170689 |
2.13.109 || STARD8 | 2.13.109 | StAR related lipid transfer domain containing 8 | 0.362554692 |
2.13.109 || TMEM70 | 2.13.109 | transmembrane protein 70 | 0.342005197 | 2.13.109 || TSC22D4 |
2.13.109 | TSC22 domain family member 4 | 0.32029484 | 2.13.109 || UST | 2.13.109 | uronyl-2-
sulfotransferase | 1.849184976 | 2.13.109 || ZBTB20 | 2.13.109 | zinc finger and BTB domain
containing 20 | 0.518239496 | 2.13.109 || ZNF542P | 2.13.109 | zinc finger protein 542, pseudogene
| 0.215619471 | 2.13.109 || ZNF70 | 2.13.109 | zinc finger protein 70 | 0.68226777 | 2.13.109 ||
ZNF844 | 2.13.109 | zinc finger protein 844 | 0.521160528 | 2.13.109 || ZNF880 | 2.13.109 | zinc
finger protein 880 | 0.77964843 | 2.13.109 || ACSM5 | 2.13.111 | acyl-CoA synthetase medium-
chain family member 5 | 0.683890782 | 2.13.111 || ADRBK1 | 2.13.111 | adrenergic, beta, receptor
kinase 1 | 0.557067614 | 2.13.111 || AEBP2 | 2.13.111 | AE binding protein 2 | 0.324321466 |
2.13.111 || AGMAT | 2.13.111 | agmatinase | 0.606004319 | 2.13.111 || ANKIB1 | 2.13.111 | ankyrin
repeat and IBR domain containing 1 | 0.251107709 | 2.13.111 || ARSA | 2.13.111 | arylsulfatase A |
0.750160235 | 2.13.111 || B3GALNT1 | 2.13.111 | beta-1,3-N-acetylgalactosaminyltransferase 1
(globoside blood group) | 0.780057 | 2.13.111 || C3orf38 | 2.13.111 | chromosome 3 open reading
frame 38 | 0.507900912 | 2.13.111 || C5AR1 | 2.13.111 | complement component 5a receptor 1 |
0.611938719 | 2.13.111 || CDH23 | 2.13.111 | cadherin-related 23 | 0.235240172 | 2.13.111 || CDK2
| 2.13.111 | cyclin-dependent kinase 2 | 0.378352115 | 2.13.111 || CTBP2 | 2.13.111 | C-terminal
binding protein 2 | 0.221798346 | 2.13.111 || CTSL | 2.13.111 | cathepsin L | 0.791556213 | 2.13.111
|| DNM2 | 2.13.111 | dynamin 2 | 0.307178926 | 2.13.111 || DTD2 | 2.13.111 | D-tyrosyl-tRNA
deacylase 2 (putative) | 0.414817763 | 2.13.111 || EGR1 | 2.13.111 | early growth response 1 |
2.184401604 | 2.13.111 || F8 | 2.13.111 | coagulation factor VIII | 0.969874965 | 2.13.111 ||
FAM172A | 2.13.111 | family with sequence similarity 172 member A | 0.765955214 | 2.13.111 ||
HCK | 2.13.111 | HCK proto-oncogene, Src family tyrosine kinase | 0.591574122 | 2.13.111 ||
HOXA3 | 2.13.111 | homeobox A3 | 0.456875599 | 2.13.111 || HSPA12A | 2.13.111 | heat shock
protein family A (Hsp70) member 12A | 0.473957291 | 2.13.111 || HTR2A | 2.13.111 | 5-
hydroxytryptamine receptor 2A | 0.430489177 | 2.13.111 || IDH2 | 2.13.111 | isocitrate
dehydrogenase 2 (NADP+), mitochondrial | 0.630488179 | 2.13.111 || IGF2BP2 | 2.13.111 | insulin
like growth factor 2 mRNA binding protein 2 | 0.954990871 | 2.13.111 || IGSF10 | 2.13.111 |
immunoglobulin superfamily member 10 | 0.878588832 | 2.13.111 || INO80D | 2.13.111 | INO80
complex subunit D | 0.971483252 | 2.13.111 || KBTBD7 | 2.13.111 | kelch repeat and BTB domain
containing 7 | 0.6206987 | 2.13.111 || KIFIC | 2.13.111 | kinesin family member 1C | 0.499234725 |
2.13.111 || LAT2 | 2.13.111 | linker for activation of T-cells family member 2 | 0.574086734 |
2.13.111 || MAPKAP1 | 2.13.111 | mitogen-activated protein kinase associated protein 1 |
0.334436347 | 2.13.111 || MIR99AHG | 2.13.111 | mir-99a-let-7c cluster host gene | 4.859450805 |
2.13.111 || MPZL2 | 2.13.111 | myelin protein zero like 2 | 0.894110176 | 2.13.111 || MRPS30 |
2.13.111 | mitochondrial ribosomal protein S30 | 0.249330581 | 2.13.111 || MS4A6A | 2.13.111 |
membrane spanning 4-domains A6A | 0.363578436 | 2.13.111 || MYRIP | 2.13.111 | myosin VIIA
and Rab interacting protein | 2.974821829 | 2.13.111 || NAPIL3 | 2.13.111 | nucleosome assembly
protein 1 like 3 | 1.765809462 | 2.13.111 || NEDD4L | 2.13.111 | neural precursor cell expressed,
developmentally down-regulated 4-like, E3 ubiquitin protein ligase | 0.812970685 | 2.13.111 ||
NEGR1 | 2.13.111 | neuronal growth regulator 1 | 4.682478671 | 2.13.111 || NFIA | 2.13.111 |
nuclear factor I/A | 1.269390224 | 2.13.111 || NFIB | 2.13.111 | nuclear factor I/B | 1.594047921 |
2.13.111 || NOVA1 | 2.13.111 | neuro-oncological ventral antigen 1 | 2.94232798 | 2.13.111 ||
NTRK2 | 2.13.111 | neurotrophic tyrosine kinase, receptor, type 2 | 4.633944435 | 2.13.111 || OMA1
|2.13.111 | OMA1 zinc metallopeptidase | 0.277222184 | 2.13.111 | PARP10 | 2.13.111 |
poly(ADP-ribose) polymerase family member 10 | 0.543773678 | 2.13.111 | PBX1 | 2.13.111 | pre-
B-cell leukemia homeobox 1 | 0.688052051 | 2.13.111 || PCSK5 | 2.13.111 | proprotein convertase
subtilisin/kexin type 5 | 0.734443093 | 2.13.111 || PCYOXIL | 2.13.111 | prenylcysteine oxidase 1
like | 0.284605579 | 2.13.111 || PLSCR4 | 2.13.111 | phospholipid scramblase 4 | 0.947501888 |
2.13.111 || PPL | 2.13.111 | periplakin | 2.509024142 | 2.13.111 || PRICKLE1 | 2.13.111 | prickle
planar cell polarity protein 1 | 0.522802685 | 2.13.111 || RBMS3 | 2.13.111 | RNA binding motif,
single stranded interacting protein 3 | 2.229894728 | 2.13.111 || RFX7 | 2.13.111 | regulatory factor
X7 | 0.222841625 | 2.13.111 || RGS14 | 2.13.111 | regulator of G-protein signaling 14 | 0.262440781
| 2.13.111 || RGS19 | 2.13.111 | regulator of G-protein signaling 19 | 0.373091892 | 2.13.111 ||
RNPC3 | 2.13.111 | RNA binding region (RNP1, RRM) containing 3 | 0.431345942 | 2.13.111 ||
RTN1 | 2.13.111 | reticulon 1 | 1.017538562 | 2.13.111 || SLC25A37 | 2.13.111 | solute carrier
family 25 member 37 | 0.814925352 | 2.13.111 || SLC25A45 | 2.13.111 | solute carrier family 25
member 45 | 0.265283041 | 2.13.111 || SLC4A4 | 2.13.111 | solute carrier family 4 member 4 |
0.349407919 | 2.13.111 || SOCS5 | 2.13.111 | suppressor of cytokine signaling 5 | 0.274385882 |
2.13.111 || SOWAHC | 2.13.111 | sosondowah ankyrin repeat domain family member C |
0.471996181 | 2.13.111 || SPPL2A | 2.13.111 | signal peptide peptidase like 2A | 0.293854081 |
2.13.111 || STK3 | 2.13.111 | serine/threonine kinase 3 | 0.325402616 | 2.13.111 || STON1 | 2.13.111
| stonin 1 | 1.356403913 | 2. 13.111 || SYNJ2 | 2. 13.111 | synaptojanin 2 | 1.185253446 | 2.13.111 ||
TRHDE-AS1 | 2.13.111 | TRHDE antisense RNA 1 | 0.962822387 | 2.13.111 || TRIM2 | 2.13.111 |
tripartite motif containing 2 | 2.395669817 | 2.13.111 || TSHZ2 | 2.13.111 | teashirt zinc finger
homeobox 2 | 1.052241312 | 2.13.111 || TSHZ3 | 2.13.111 | teashirt zinc finger homeobox 3 |
0.887191214 | 2.13.111 || TULP3 | 2.13.111 | tubby like protein 3 | 0.97655164 | 2.13.111 || USB1 |
2.13.111 | U6 snRNA biogenesis 1 | 0.252964287 | 2.13.111 || WASF3 | 2.13.111 | WAS protein
family member 3 | 1.618709402 | 2.13.111 || WIPI1 | 2.13.111 | WD repeat domain,
phosphoinositide interacting 1 | 0.308108308 | 2.13.111 || XG | 2.13.111 | Xg blood group |
1.753857649 | 2.13.111 || ZBTB10 | 2.13.111 | zinc finger and BTB domain containing 10 |
0.855741505 | 2.13.111 || ACP2 | 2.13.112 | acid phosphatase 2, lysosomal | 0.664126978 | 2.13.112
|| ARHGAP12 | 2.13.112 | Rho GTPase activating protein 12 | 0.233692783 | 2.13.112 || ARRB2 |
2.13.112 | arrestin, beta 2 | 0.667353075 | 2.13.112 || BLNK | 2.13.112 | B-cell linker | 0.435714403
| 2.13.112 || C10orf10 | 2.13.112 | chromosome 10 open reading frame 10 | 1.450562961 | 2.13.112 ||
C9orf40 | 2.13.112 | chromosome 9 open reading frame 40 | 0.436229202 | 2. 13.112 || CD58 |
2.13.112 | CD58 molecule | 0.222572712 | 2.13.112 || CD74 | 2.13.112 | CD74 molecule |
0.237274973 | 2.13.112 || CLDN11 | 2.13.112 | claudin 11 | 1.352899095 | 2.13.112 || COTL1 |
2.13.112 | coactosin-like F-actin binding protein 1 | 0.419338939 | 2.13.112 || CPNE1 | 2.13.112 |
copine 1 | 0.306504255 | 2.13.112 || CSPP1 | 2.13.112 | centrosome and spindle pole associated
protein 1 | 0.298382526 | 2.13.112 || CTSB | 2.13.112 | cathepsin B | 0.444678736 | 2.13.112 ||
DAPK1 | 2.13.112 | death-associated protein kinase 1 | 0.355467029 | 2.13.112 || DOK2 | 2.13.112 |
docking protein 2 | 0.406246015 | 2.13.112 || FAM105A | 2.13.112 | family with sequence similarity
105 member A | 0.379660874 | 2.13.112 || FAM96A | 2.13.112 | family with sequence similarity 96
member A | 0.240033343 | 2.13.112 || FCERIG | 2.13.112 | Fc fragment of IgE receptor Ig |
0.777975804 | 2.13.112 || FHL5 | 2.13.112 | four and a half LIM domains 5 | 1.775450295 | 2.13.112
|| FMNL1 | 2.13.112 | formin like 1 | 0.645099805 | 2.13.112 || FTH1 | 2.13.112 | ferritin, heavy
polypeptide 1 | 0.235701696 | 2.13.112 || GHDC | 2.13.112 | GH3 domain containing | 0.254119438
| 2.13.112 || HACD3 | 2.13.112 | 3-hydroxyacyl-CoA dehydratase 3 | 0.366670218 | 2.13.112 ||
HECW2 | 2.13.112 | HECT, C2 and WW domain containing E3 ubiquitin protein ligase 2 |
1.386960007 | 2.13.112 || HK3 | 2.13.112 | hexokinase 3 | 1.301991786 | 2.13.112 || IL13RA2 |
2.13.112 | interleukin 13 receptor subunit alpha 2 | 2.550295806 | 2.13.112 || INPP4B | 2.13.112 |
inositol polyphosphate-4-phosphatase type II B | 1.917873318 | 2.13.112 || LAPTM5 | 2.13.112 |
lysosomal protein transmembrane 5 | 0.463046131 | 2.13.112 || LIFR | 2.13.112 | leukemia
inhibitory factor receptor alpha | 2.429454608 | 2.13.112 || LRRC16A | 2.13.112 | leucine rich repeat
containing 16A | 0.908080901 | 2.13.112 || LTA4H | 2.13.112 | leukotriene A4 hydrolase |
0.72641255 | 2.13.112 || MAN2B1 | 2.13.112 | mannosidase alpha class 2B member 1 |
0.467641762 | 2.13.112 || MAP1B | 2.13.112 | microtubule associated protein 1B | 1.701090816 |
2.13.112 || MFSD1 | 2.13.112 | major facilitator superfamily domain containing 1 | 0.240993841 |
2.13.112 || MSH2 | 2.13.112 | mutS homolog 2 | 0.505425044 | 2.13.112 || NAGA | 2.13.112 | N-
acetylgalactosaminidase, alpha- | 0.418428557 | 2.13.112 || NET1 | 2.13.112 | neuroepithelial cell
transforming 1 | 0.867597081 | 2.13.112 || NR1H3 | 2.13.112 | nuclear receptor subfamily 1 group H
member 3 | 0.772364085 | 2.13.112 || ODF2L | 2.13.112 | outer dense fiber of sperm tails 2 like |
0.289734932 | 2.13.112 || OSGEPL1 | 2.13.112 | O-sialoglycoprotein endopeptidase-like 1 |
0.601682073 | 2.13.112 || P2RX4 | 2.13.112 | purinergic receptor P2X 4 | 0.778628666 | 2.13.112 ||
PARP3 | 2.13.112 | poly(ADP-ribose) polymerase family member 3 | 0.264796188 | 2.13.112 ||
PEX12 | 2.13.112 | peroxisomal biogenesis factor 12 | 0.563432071 | 2.13.112 || PILRA | 2.13.112 |
paired immunoglobin-like type 2 receptor alpha | 0.994717293 | 2.13.112 || PTGER2 | 2.13.112 |
prostaglandin E receptor 2 | 0.628419606 | 2.13.112 || PTPN13 | 2.13.112 | protein tyrosine
phosphatase, non-receptor type 13 | 0.881444917 | 2.13.112 || PUS7 | 2.13.112 | pseudouridylate
synthase 7 (putative) | 0.302191589 | 2.13.112 || RAB30 | 2.13.112 | RAB30, member RAS
oncogene family | 1.077446891 | 2.13.112 || RGCC | 2.13.112 | regulator of cell cycle | 0.714836677
| 2.13.112 | RNF166 | 2.13.112 | ring finger protein 166 | 0.238032034 | 2.13.112 || RNPEP |
2.13.112 | arginyl aminopeptidase | 0.214773118 | 2.13.112 || SEMA6A | 2.13.112 | semaphorin 6A |
1.425697763 | 2.13.112 || SENP7 | 2.13.112 | SUMO1/sentrin specific peptidase 7 | 0.340935043 |
2.13.112 || SETD9 | 2.13.112 | SET domain containing 9 | 0.43506454 | 2.13.112 || SLC15A3 |
2.13.112 | solute carrier family 15 member 3 | 0.867682008 | 2.13.112 || SLC25A19 | 2.13.112 |
solute carrier family 25 member 19 | 1.556127813 | 2.13.112 || SLCO2B1 | 2.13.112 | solute carrier
organic anion transporter family member 2B1 | 0.464845675 | 2.13.112 || SPI1 | 2.13.112 | Spi-1
proto-oncogene | 1.01704837 | 2.13.112 || SYNPO2 | 2.13.112 | synaptopodin 2 | 3.221199695 |
2.13.112 || TAB1 | 2.13.112 | TGF-beta activated kinase 1/MAP3K7 binding protein 1 |
0.241853879 | 2.13.112 || TFEC | 2.13.112 | transcription factor EC | 1.194345762 | 2.13.112 ||
TM6SF1 | 2.13.112 | transmembrane 6 superfamily member 1 | 0.240612663 | 2.13.112 || TNFAIP2
| 2.13.112 | TNF alpha induced protein 2 | 0.51361715 | 2.13.112 || TNFSF13 | 2.13.112 | tumor
necrosis factor superfamily member 13 | 0.45299741 | 2.13.112 || TYROBP | 2.13.112 | TYRO
protein tyrosine kinase binding protein | 0.395515118 | 2.13.112 || UNC93B1 | 2.13.112 | unc-93
homolog B1 (C. elegans) | 0.897472824 | 2.13.112 || VAMP8 | 2.13.112 | vesicle associated
membrane protein 8 | 0.447244737 | 2.13.112 || VPS9D1 | 2.13.112 | VPS9 domain containing 1 |
0.442328486 | 2.13.112 || WRAP73 | 2.13.112 | WD repeat containing, antisense to TP73 |
0.273668604 | 2.13.112 || YWHAH | 2.13.112 | tyrosine 3-monooxygenase/tryptophan 5-
monooxygenase activation protein eta | 0.33826808 | 2.13.112 || ZC2HC1A | 2.13.112 | zinc finger
C2HC-type containing 1A | 0.324258645 | 2.13.112 || ZNF260 | 2.13.112 | zinc finger protein 260 |
0.224175965 | 2.13.112 || ABCA7 | 2.13.114 | ATP binding cassette subfamily A member 7 |
0.242566393 | 2.13.114 || ABCD1 | 2.13.114 | ATP binding cassette subfamily D member 1 |
0.3229188 | 2.13.114 || ABHD12 | 2.13.114 | abhydrolase domain containing 12 | 0.39543476 |
2.13.114 || ADPGK | 2.13.114 | ADP-dependent glucokinase | 0.292056261 | 2.13.114 || AGTRAP |
2.13.114 | angiotensin II receptor associated protein | 0.31645459 | 2.13.114 || AP1B1 | 2.13.114 |
adaptor related protein complex 1 beta 1 subunit | 0.313746684 | 2.13.114 || ARAP1 | 2.13.114 |
ArfGAP with RhoGAP domain, ankyrin repeat and PH domain 1 | 0.270031263 | 2.13.114 ||
ARHGDIA | 2.13.114 | Rho GDP dissociation inhibitor (GDI) alpha | 0.344731536 | 2.13.114 ||
ARHGEF28 | 2.13.114 | Rho guanine nucleotide exchange factor 28 | 0.276366556 | 2.13.114 ||
ARPC4 | 2.13.114 | actin related protein 2/3 complex subunit 4 | 0.338889024 | 2.13.114 || ATP1A1
| 2.13.114 | ATPase Na+/K+ transporting subunit alpha 1 | 0.256338538 | 2.13.114 || ATP6AP1 |
2.13.114 | ATPase H+ transporting accessory protein 1 | 0.385387572 | 2.13.114 || ATP6V0C |
2.13.114 | ATPase H+ transporting V0 subunit c | 0.258838881 | 2.13.114 || BMF | 2.13.114 | Bcl2
modifying factor | 0.408173976 | 2.13.114 || BRI3 | 2.13.114 | brain protein 13 | 0.276505057 |
2.13.114 || C11orf74 | 2.13.114 | chromosome 11 open reading frame 74 | 0.356840888 | 2.13.114 ||
CBLB | 2.13.114 | Cbl proto-oncogene B, E3 ubiquitin protein ligase | 0.6990346 | 2.13.114 ||
CCDC71L | 2.13.114 | coiled-coil domain containing 71-like | 0.447881502 | 2.13.114 || CD33 |
2.13.114 | CD33 molecule | 0.458006501 | 2.13.114 || CD99P1 | 2.13.114 | CD99 molecule
pseudogene 1 | 0.975712846 | 2.13.114 || CDC42 | 2.13.114 | cell division cycle 42 | 0.249055075 |
2.13.114 || CDK6 | 2.13.114 | cyclin-dependent kinase 6 | 0.313501183 | 2.13.114 || CEBPD |
2.13.114 | CCAAT/enhancer binding protein delta | 0.311601959 | 2.13.114 || CHKA | 2.13.114 |
choline kinase alpha | 0.242642167 | 2.13.114 || CHSY3 | 2.13.114 | chondroitin sulfate synthase 3 |
0.50778913 | 2.13.114 || CKS2 | 2.13.114 | CDC28 protein kinase regulatory subunit 2 |
1.100558823 | 2.13.114 || COMT | 2.13.114 | catechol-O-methyltransferase | 0.240369811 | 2.13.114
|| CTNNAL1 | 2.13.114 | catenin alpha-like 1 | 0.941381818 | 2.13.114 || DAGLB | 2.13.114 |
diacylglycerol lipase beta | 0.412141118 | 2.13.114 || DHRS7 | 2.13.114 | dehydrogenase/reductase
(SDR family) member 7 | 0.227646413 | 2.13.114 || ECT2 | 2.13.114 | epithelial cell transforming 2 |
0.34471067 | 2.13.114 || EGR3 | 2.13.114 | early growth response 3 | 0.802268668 | 2.13.114 ||
ENPP4 | 2.13.114 | ectonucleotide pyrophosphatase/phosphodiesterase 4 (putative) | 0.459210345 |
2.13.114 || FAM109A | 2.13.114 | family with sequence similarity 109 member A | 0.277290623 |
2.13.114 || FAM129B | 2.13.114 | family with sequence similarity 129 member B | 0.558431272 |
2.13.114 || FAM53B | 2.13.114 | family with sequence similarity 53 member B | 0.220473279 |
2.13.114 || FKBP10 | 2.13.114 | FK506 binding protein 10 | 1.497576934 | 2.13.114 || FOXO4 |
2.13.114 | forkhead box 04 | 0.358715734 | 2.13.114 || GAA | 2.13.114 | glucosidase, alpha; acid |
0.86547707 | 2.13.114 || GM2A | 2.13.114 | GM2 ganglioside activator | 0.355757498 | 2.13.114 ||
GPNMB | 2.13.114 | glycoprotein nmb | 0.466321045 | 2.13.114 || GRN | 2.13.114 | granulin |
0.447978794 | 2.13.114 || HEXB | 2.13.114 | hexosaminidase subunit beta | 0.571878974 | 2.13.114
|| HOXB6 | 2.13.114 | homeobox B6 | 0.668773405 | 2.13.114 || HVCN1 | 2.13.114 | hydrogen
voltage gated channel 1 | 0.395585015 | 2.13.114 || KLF9 | 2.13.114 | Kruppel-like factor 9 |
0.553886869 | 2.13.114 || LINC00304 | 2.13.114 | long intergenic non-protein coding RNA 304 |
0.393501478 | 2.13.114 || MAD2L1 | 2.13.114 | MAD2 mitotic arrest deficient-like 1 (yeast) |
1.100419452 | 2.13.114 || MBOAT1 | 2.13.114 | membrane bound O-acyltransferase domain
containing 1 | 0.357216125 | 2.13.114 || MCTP1 | 2.13.114 | multiple C2 and transmembrane
domain containing 1 | 0.988495346 | 2.13.114 || ME1 | 2.13.114 | malic enzyme 1, NADP(+)-
dependent, cytosolic | 0.278326942 | 2.13.114 || MGAT1 | 2.13.114 | mannosyl (alpha-1,3-)-
glycoprotein beta-1,2-N-acetylglucosaminyltransferase | 0.350451991 | 2.13.114 || MINA | 2.13.114
| MYC induced nuclear antigen | 0.283548651 | 2.13.114 || MS4A14 | 2.13.114 | membrane
spanning 4-domains A14 | 0.900422952 | 2.13.114 || MVP | 2.13.114 | major vault protein |
0.242014911 | 2.13.114 || NAGPA | 2.13.114 | N-acetylglucosamine-1-phosphodiester alpha-N-
acetylglucosaminidase | 0.325417838 | 2.13.114 || P4HB | 2.13.114 | prolyl 4-hydroxylase subunit
beta | 0.342471626 | 2.13.114 || PARP6 | 2.13.114 | poly(ADP-ribose) polymerase family member 6
| 0.25692068 | 2.13.114 || PARVB | 2.13.114 | parvin beta | 0.70562519 | 2.13.114 || PDAP1 |
2.13.114 | PDGFA associated protein 1 | 0.402836026 | 2.13.114 || PIK3IP1 | 2.13.114 |
phosphoinositide-3-kinase interacting protein 1 | 0.330308467 | 2.13.114 || PIK3R5 | 2.13.114|
phosphoinositide-3-kinase regulatory subunit 5 | 0.676437862 | 2.13.114 || PKM | 2.13.114 |
pyruvate kinase, muscle | 0.485963464 | 2.13.114 || PKN2 | 2.13.114 | protein kinase N2 |
0.366914792 | 2.13.114 || PLBD1 | 2.13.114 | phospholipase B domain containing 1 | 0.798153641 |
2.13.114 || PLD3 | 2.13.114 | phospholipase D family member 3 | 0.447032144 | 2.13.114 || PLP2 |
2.13.114 | proteolipid protein 2 (colonic epithelium-enriched) | 0.241328907 | 2.13.114 || POLR3K |
2.13.114 | polymerase (RNA) III subunit K | 0.233338628 | 2.13.114 || PON2 | 2.13.114 |
paraoxonase 2 | 0.237306537 | 2.13.114 || PPDPF | 2.13.114 | pancreatic progenitor cell
differentiation and proliferation factor | 0.310586024 | 2.13.114 || PRRG1 | 2.13.114 | proline rich
Gla (G-carboxyglutamic acid) 1 | 0.653723801 | 2.13.114 || PTAFR | 2.13.114 | platelet activating
factor receptor | 0.478181238 | 2.13.114 || QSOX1 | 2.13.114 | quiescin sulfhydryl oxidase 1 |
0.368927339 | 2.13.114 || RHOQ | 2.13.114 | ras homolog family member Q | 0.241573272 |
2.13.114 || RNF130 | 2.13.114 | ring finger protein 130 | 0.3114708 | 2.13.114 || SCRN3 | 2.13.114 |
secernin 3 | 0.248950373 | 2.13.114 || SEC22B | 2.13.114 | SEC22 homolog B, vesicle trafficking
protein (gene/pseudogene) | 0.2457394 | 2.13.114 || SGOL2 | 2.13.114 | shugoshin-like 2 (S. pombe)
| 0.677440331 | 2.13.114 || SH3BGRL3 | 2.13.114 | SH3 domain binding glutamate rich protein like
3 | 0.332563528 | 2.13.114 || SIGLEC1 | 2.13.114 | sialic acid binding Ig like lectin 1 | 0.544833518
| 2.13.114 || SIRPA | 2.13.114 | signal regulatory protein alpha | 0.518829285 | 2.13.114 || SLC16A7
| 2.13.114 | solute carrier family 16 member 7 | 1.623659067 | 2. 13.114 || SLC9A6 | 2.13.114 |
solute carrier family 9 member A6 | 0.221078859 | 2.13.114 || SMPDL3A | 2.13.114 |
sphingomyelin phosphodiesterase acid like 3A | 0.31952755 | 2.13.114 || THAP8 | 2.13.114 | THAP
domain containing 8 | 0.216626417 | 2.13.114 || TMEM129 | 2.13.114 | transmembrane protein 129 |
0.263432145 | 2.13.114 || TMEM176A | 2.13.114 | transmembrane protein 176A | 0.282487188 |
2.13.114 || TMEM176B | 2.13.114 | transmembrane protein 176B | 0.316009486 | 2.13.114 ||
TMEM198B | 2.13.114 | transmembrane protein 198B (pseudogene) | 0.333031752 | 2.13.114 ||
TMEM259 | 2.13.114 | transmembrane protein 259 | 0.455797342 | 2.13.114 || TMEM64 | 2.13.114 |
transmembrane protein 64 | 0.598506919 | 2.13.114 || TMTC2 | 2.13.114 | transmembrane and
tetratricopeptide repeat containing 2 | 0.978419575 | 2.13.114 || TNK2 | 2.13.114 | tyrosine kinase,
non-receptor, 2 | 0.227917443 | 2.13.114 || TPP1 | 2.13.114 | tripeptidyl peptidase I | 0.279185597 |
2.13.114 || TRMT6 | 2.13.114 | tRNA methyltransferase 6 | 0.674126391 | 2.13.114 || TSC22D3 |
2.13.114 | TSC22 domain family member 3 | 0.831241773 | 2.13.114 || TSPAN4 | 2.13.114 |
tetraspanin 4 | 0.248755326 | 2.13.114 || TSPO | 2.13.114 | translocator protein | 0.237547491 |
2.13.114 || TTC38 | 2.13.114 | tetratricopeptide repeat domain 38 | 0.297274267 | 2.13.114 || TTC7A
| 2.13.114 | tetratricopeptide repeat domain 7A | 0.370564594 | 2.13.114 || UBE2D1 | 2.13.114 |
ubiquitin conjugating enzyme E2D 1 | 0.308882488 | 2.13.114 || ZBTB16 | 2.13.114 | zinc finger
and BTB domain containing 16 | 3.507241815 | 2.13.114 || ZDHHC7 | 2.13.114 | zinc finger
DHHC-type containing 7 | 0.267367989 | 2.13.114 || ZMYND15 | 2.13.114 | zinc finger MYND-
type containing 15 | 0.466281515 | 2.13.114 || ZNF138 | 2.13.114 | zinc finger protein 138 |
0.308645533 | 2.13.114 || ANKRD44 | 2.13.117 | ankyrin repeat domain 44 | 0.419715692 |
2.13.117 || ARHGAP30 | 2.13.117 | Rho GTPase activating protein 30 | 0.553082233 | 2.13.117 ||
ATP6V1B2 | 2.13.117 | ATPase H+ transporting V1 subunit B2 | 0.372650633 | 2.13.117 || C2orf76
| 2.13.117 | chromosome 2 open reading frame 76 | 0.779841415 | 2.13.117 || CDC25B | 2.13.117 |
cell division cycle 25B | 0.234935615 | 2.13.117 || CHST11 | 2.13.117 | carbohydrate (chondroitin
4) sulfotransferase 11 | 0.741694504 | 2.13.117 || DOK3 | 2.13.117 | docking protein 3 |
1.407402243 | 2.13.117 || EVL | 2.13.117 | Enah/Vasp-like | 0.51540568 | 2.13.117 || FAM229B |
2.13.117 | family with sequence similarity 229 member B | 0.620487724 | 2.13.117 || IL15 |
2.13.117 | interleukin 15 | 0.814306198 | 2.13.117 || IL17RA | 2.13.117 | interleukin 17 receptor A |
0.455702783 | 2.13.117 || IRF8 | 2.13.117 | interferon regulatory factor 8 | 0.516252625 | 2.13.117 ||
KIAA1468 | 2.13.117 | KIAA1468 | 0.253685207 | 2.13.117 || LRIF1 | 2.13.117 | ligand dependent
nuclear receptor interacting factor 1 | 0.503312043 | 2.13.117 || MEF2A | 2.13.117 | myocyte
enhancer factor 2A | 0.350986547 | 2.13.117 || MGME1 | 2.13.117 | mitochondrial genome
maintenance exonuclease 1 | 0.220814438 | 2.13.117 || NPC1 | 2.13.117 | Niemann-Pick disease,
type C1 | 0.659163398 | 2.13.117 || NUP62 | 2.13.117 | nucleoporin 62kDa | 0.385630702 | 2.13.117
|| OSGIN2 | 2.13.117 | oxidative stress induced growth inhibitor family member 2 | 0.485072903 |
2.13.117 || PREX1 | 2.13.117 | phosphatidylinositol-3,4,5-trisphosphate-dependent Rac exchange
factor 1 | 0.250217617 | 2.13.117 || PTPRJ | 2.13.117 | protein tyrosine phosphatase, receptor type J |
0.758561814 | 2.13.117 || RAPGEF1 | 2.13.117 | Rap guanine nucleotide exchange factor 1 |
0.572073564 | 2.13.117 || RBM47 | 2.13.117 | RNA binding motif protein 47 | 0.486946946 |
2.13.117 || RUNX3 | 2.13.117 | runt related transcription factor 3 | 1.275263144 | 2.13.117 || SDC3 |
2.13.117 | syndecan 3 | 0.223505318 | 2.13.117 || SEPT6 | 2.13.117 | septin 6 | 0.311496997 |
2.13.117 || SFRP2 | 2.13.117 | secreted frizzled-related protein 2 | 4.809938046 | 2.13.117 || SP140L
| 2.13.117 | SP140 nuclear body protein like | 0.290145129 | 2. 13.117 || TNS3 | 2. 13.117 | tensin 3 |
0.31387926 | 2.13.117 || TUBB2A | 2.13.117 | tubulin beta 2A class IIa | 0.325023776 | 2.13.117 ||
WDFY4 | 2.13.117 | WDFY family member 4 | 0.310390837 | 2.13.117 || ZC3H12D | 2.13.117 |
zinc finger CCCH-type containing 12D | 0.684374131 | 2.13.117 || ARHGAP9 | 2.16.121 | Rho
GTPase activating protein 9 | 0.518721369 | 2.16.121 || CCR2 | 2.16.121 | chemokine (C-C motif)
receptor 2 | 2.238090097 | 2.16.121 || CD48 | 2.16.121 | CD48 molecule | 1.237135937 | 2.16.121 ||
CD52 | 2.16.121 | CD52 molecule | 1.862049647 | 2.16.121 || CDC42SE2 | 2.16.121 | CDC42 small
effector 2 | 0.306215443 | 2.16.121 || CORO1A | 2.16.121 | coronin 1A | 1.034299612 | 2.16.121 ||
DSTYK | 2.16.121 | dual serine/threonine and tyrosine protein kinase | 0.335087459 | 2.16.121 || F5
| 2.16.121 | coagulation factor V | 2.475642818 | 2.16.121 || FCN1 | 2.16.121 | ficolin 1 |
3.497920465 | 2.16.121 || FGD3 | 2.16.121 | FYVE, RhoGEF and PH domain containing 3 |
0.806294346 | 2.16.121 || IKZF1 | 2.16.121 | IKAROS family zinc finger 1 | 0.384997667 | 2.16.121
|| IL2RG | 2.16.121 | interleukin 2 receptor subunit gamma | 0.937783702 | 2.16.121 || ITGAL |
2.16.121 | integrin subunit alpha L | 1.473962494 | 2.16.121 || LRCH4 | 2.16.121 | leucine-rich
repeats and calponin homology (CH) domain containing 4 | 0.27162551 | 2.16.121 || MLKL |
2.16.121 | mixed lineage kinase domain-like | 0.234233086 | 2.16.121 || NAPIL5 | 2.16.121 |
nucleosome assembly protein 1 like 5 | 0.807252711 | 2.16.121 || PRKCB | 2.16.121 | protein kinase
C beta | 1.957382364 | 2.16.121 || RAC2 | 2.16.121 | ras-related C3 botulinum toxin substrate 2 (rho
family, small GTP binding protein Rac2) | 1.353567681 | 2.16.121 || RASSF5 | 2.16.121 | Ras
association domain family member 5 | 0.766637803 | 2.16.121 || SASH3 | 2.16.121 | SAM and SH3
domain containing 3 | 0.817474107 | 2.16.121 || SEMA4D | 2.16.121 | semaphorin 4D |
1.778919845 | 2.16.121 || SLAMF7 | 2.16.121 | SLAM family member 7 | 1.569509011 | 2.16.121 ||
TMC8 | 2.16.121 | transmembrane channel like 8 | 0.597734204 | 2.16.121 || TRIM6 | 2.16.121 |
tripartite motif containing 6 | 0.524072509 | 2.16.121 || BCR | 2.16.122 | breakpoint cluster region |
0.415560216 | 2.16.122 || C17orf62 | 2.16.122 | chromosome 17 open reading frame 62 |
0.255637923 | 2.16.122 || CSF2RB | 2.16.122 | colony stimulating factor 2 receptor beta common
subunit | 0.818078341 | 2.16.122 || DNASEIL3 | 2.16.122 | deoxyribonuclease I-like 3 |
2.214070063 | 2.16.122 || DOCK8 | 2.16.122 | dedicator of cytokinesis 8 | 0.42091134 | 2.16.122 ||
FOXP2 | 2.16.122 | forkhead box P2 | 2.156829532 | 2.16.122 || GPR160 | 2.16.122 | G protein-
coupled receptor 160 | 1.441278593 | 2.16.122 || GPRASP1 | 2.16.122 | G protein-coupled receptor
associated sorting protein 1 | 1.115161134 | 2.16.122 || HCST | 2.16.122 | hematopoietic cell signal
transducer | 1.151591744 | 2.16.122 || HTR2B | 2.16.122 | 5-hydroxytryptamine receptor 2B |
1.496781013 | 2.16.122 || IER5 | 2.16.122 | immediate early response 5 | 0.358472406 | 2.16.122 ||
IGF1 | 2.16.122 | insulin like growth factor 1 | 1.754852301 | 2.16.122 || ITGB2-AS1 | 2.16.122 |
ITGB2 antisense RNA 1 | 1.721017218 | 2.16.122 || KAZN | 2.16.122 | kazrin, periplakin
interacting protein | 0.929325905 | 2.16.122 || LCP1 | 2.16.122 | lymphocyte cytosolic protein 1 (L-
plastin) | 1.068888034 | 2.16.122 || MYOIF | 2.16.122 | myosin IF | 0.503103547 | 2.16.122 ||
NBEAL2 | 2.16.122 | neurobeachin like 2 | 0.410898235 | 2.16.122 || NPR3 | 2.16.122 | natriuretic
peptide receptor 3 | 1.737416682 | 2.16.122 || RASSF2 | 2.16.122 | Ras association domain family
member 2 | 0.41778909 | 2.16.122 || RCAN3 | 2.16.122 | RCAN family member 3 | 0.307824209 |
2.16.122 || RFX5 | 2.16.122 | regulatory factor X5 | 0.305684472 | 2.16.122 || RGMA | 2.16.122 |
repulsive guidance molecule family member a | 2.014608159 | 2.16.122 || SIPR3 | 2.16.122 |
sphingosine-1-phosphate receptor 3 | 1.798519463 | 2.16.122 || SECTM1 | 2.16.122 | secreted and
transmembrane 1 | 0.981491923 | 2.16.122 || SH3RF1 | 2.16.122 | SH3 domain containing ring
finger 1 | 0.910632771 | 2.16.122 || SOX9 | 2.16.122 | SRY-box 9 | 0.952326647 | 2.16.122 ||
SPON1 | 2.16.122 | spondin 1 | 2.599595358 | 2.16.122 || SRGN | 2.16.122 | serglycin | 0.356178993
| 2.16.122 | STK10 | 2.16.122 | serine/threonine kinase 10 | 0.302351805 | 2.16.122 || STK38 |
2.16.122 | serine/threonine kinase 38 | 0.233097472 | 2.16.122 || TAGAP | 2.16.122 | T-cell
activation RhoGTPase activating protein | 0.84233511 | 2.16.122 || TERT | 2.16.122 | telomerase
reverse transcriptase | 0.293475695 | 2.16.122 || TIGD1 | 2.16.122 | tigger transposable element
derived 1 | 0.229965831 | 2.16.122 || TLR8 | 2.16.122 | toll like receptor 8 | 0.706115783 | 2.16.122
|| TUBB2B | 2.16.122 | tubulin beta 2B class IIb | 2.431655015 | 2.16.122 || ACKR1 | 2.17 | atypical
chemokine receptor 1 (Duffy blood group) | 1.453950152 | 2.17.125 || ACVRL1 | 2.17 | activin A
receptor like type 1 | 0.457941698 | 2.17.126 || ADAMTS16 | 2.17 | ADAM metallopeptidase with
thrombospondin type 1 motif 16 | 0.594432283 | 2.17.124 || ADAMTS9 | 2.17 | ADAM
metallopeptidase with thrombospondin type 1 motif 9 | 0.600967661 | 2.17.126 || ADCY1 | 2.17 |
adenylate cyclase 1 (brain) | 1.589641497 | 2.17.124 || ADGRF5 | 2.17 | adhesion G protein-coupled
receptor F5 | 1.04552692 | 2.17.125 || ADGRG1 | 2.17 | adhesion G protein-coupled receptor G1 |
0.847277013 | 2.17.126 || ADGRL2 | 2.17 | adhesion G protein-coupled receptor L2 | 1.122101436 |
2.17.125 || ADGRL4 | 2.17 | adhesion G protein-coupled receptor L4 | 1.352331518 | 2.17.125 ||
AKR7A2 | 2.17 | aldo-keto reductase family 7, member A2 | 0.214630329 | 2.17.124 || ANKRD29 |
2.17 | ankyrin repeat domain 29 | 1.095693821 | 2.17.125 || ANKRD50 | 2.17 | ankyrin repeat
domain 50 | 0.978119219 | 2.17.127 || APBBIIP | 2.17 | amyloid beta precursor protein binding
family B member 1 interacting protein | 0.237345876 | 2.17.124 || ARHGAP29 | 2.17 | Rho GTPase
activating protein 29 | 1.21795146 | 2.17.125 || ARL15 | 2.17 | ADP ribosylation factor like GTPase
15 | 0.322412003 | 2.17.124 || AVPRIA | 2.17 | arginine vasopressin receptor 1A | 1.752850075 |
2.17.124 || BACE2 | 2.17 | beta-site APP-cleaving enzyme 2 | 0.51524745 | 2.17.124 || BASP1 | 2.17
| brain abundant membrane attached signal protein 1 | 0.427576478 | - || C11orf45 | 2.17 |
chromosome 11 open reading frame 45 | 0.471956294 | 2.17.124 || Clorf115 | 2.17 | chromosome 1
open reading frame 115 | 2.391779001 | 2.17.125 || Clorf162 | 2.17 | chromosome 1 open reading
frame 162 | 0.804747996 | 2.17.124 || C21orf91 | 2.17 | chromosome 21 open reading frame 91 |
0.419842289 | 2.17.124 || C3orf70 | 2.17 | chromosome 3 open reading frame 70 | 0.586569523 |
2.17.124 || C8orf4 | 2.17 | chromosome 8 open reading frame 4 | 2.034107668 | 2.17.124 || CALCRL
| 2.17 | calcitonin receptor like receptor | 1.054186733 | 2.17.125 || CC2DIA | 2.17 | coiled-coil and
C2 domain containing 1A | 0.221733565 | 2.17.124 || CCNG2 | 2.17 | cyclin G2 | 0.220557952 |
2.17.125 || CD200 | 2.17 | CD200 molecule | 1.652979997 | - || CD24 | 2.17 | CD24 molecule |
0.894409745 | 2.17.125 || CD34 | 2.17 | CD34 molecule | 1.288607951 | 2.17.125 || CDH13 | 2.17 |
cadherin 13 | 0.579778488 | 2.17.127 || CEP112 | 2.17 | centrosomal protein 112kDa | 1.319818315 |
2.17.124 || CHML | 2.17 | choroideremia-like (Rab escort protein 2) | 0.519521543 | 2.17.125 ||
CHN1 | 2.17 | chimerin 1 | 0.808407772 | - || CLIP4 | 2.17 | CAP-Gly domain containing linker
protein family member 4 | 0.494595917 | - || COBLL1 | 2.17 | cordon-bleu WH2 repeat protein like
1 | 0.974430835 | 2.17.124 || COL15A1 | 2.17 | collagen type XV alpha 1 | 0.844352503 | 2.17.126 ||
COL21A1 | 2.17 | collagen type XXI alpha 1 | 1.421131949 | 2.17.124 || COL4A1 | 2.17 | collagen
type IV alpha 1 | 0.491598467 | 2.17.126 || COL4A2 | 2.17 | collagen type IV alpha 2 | 0.537549227
| 2.17.126 || COQ2 | 2.17 | coenzyme Q2 4-hydroxybenzoate polyprenyltransferase | 0.389520277 |
2.17.124 || CPNE2 | 2.17 | copine 2 | 0.312011555 | 2.17.126 || CTDSPL | 2.17 | CTD small
phosphatase like | 0.216582787 | 2.17.125 || CYBA | 2.17 | cytochrome b-245, alpha polypeptide |
0.273760957 | 2.17.124 | CYGB | 2.17 | cytoglobin | 0.278587725 | 2.17.124 || CYYR1 | 2.17 |
cysteine/tyrosine-rich 1 | 1.907969486 | 2.17.124 || DDX6 | 2.17 | DEAD-box helicase 6 |
0.235178602 | 2.17.127 || DNAJC12 | 2.17 | DnaJ heat shock protein family (Hsp40) member C12 |
0.416158071 | 2.17.126 || DOCK6 | 2.17 | dedicator of cytokinesis 6 | 0.352211575 | 2.17.126 ||
EBF3 | 2.17 | early B-cell factor 3 | 0.821326334 | 2.17.124 || ECM2 | 2.17 | extracellular matrix
protein 2, female organ and adipocyte specific | 0.48635108 | 2.17.125 || EDNRA | 2.17 | endothelin
receptor type A | 0.941379977 | 2.17.127 | EIF2B4 | 2.17 | eukaryotic translation initiation factor 2B
subunit delta | 0.244574254 | 2.17.125 || EIF2D | 2.17 | eukaryotic translation initiation factor 2D |
0.227236737 | 2.17.124 || EMCN | 2.17 | endomucin | 1.495883234 | 2.17.124 || EMP3 | 2.17 |
epithelial membrane protein 3 | 0.409891746 | 2.17.125 || ENPEP | 2.17 | glutamyl aminopeptidase |
0.883521596 | 2.17.126 || ENPP2 | 2.17 | ectonucleotide pyrophosphatase/phosphodiesterase 2 |
1.098641394 | 2.17.124 || EPAS1 | 2.17 | endothelial PAS domain protein 1 | 0.453120964 |
2.17.125 || EPB41L4A | 2.17 | erythrocyte membrane protein band 4.1 like 4A | 0.404663911 |
2.17.125 || ETS2 | 2.17 | ETS proto-oncogene 2, transcription factor | 0.239877709 | 2.17.125 || F2R
| 2.17 | coagulation factor II thrombin receptor | 1.688783268 | 2.17.125 || FAM129A | 2.17 | family
with sequence similarity 129 member A | 0.346193165 | 2.17.126 || FAM160B1 | 2.17 | family with
sequence similarity 160 member B1 | 0.320470916 | 2.17.125 || FAM162B | 2.17 | family with
sequence similarity 162 member B | 0.577189799 | 2.17.125 || FAM171B | 2.17 | family with
sequence similarity 171 member B | 1.119407096 | 2.17.124 || FAM200A | 2.17 | family with
sequence similarity 200 member A | 0.224937704 | 2.17.124 || FAM43A | 2.17 | family with
sequence similarity 43 member A | 1.677486112 | 2.17.125 || FAM84B | 2.17 | family with sequence
similarity 84 member B | 1.449202678 | 2.17.125 || FBLIM1 | 2.17 | filamin binding LIM protein 1 |
0.41760694 | - || FGD6 | 2.17 | FYVE, RhoGEF and PH domain containing 6 | 0.358933985 | - ||
FHL2 | 2.17 | four and a half LIM domains 2 | 0.997569729 | 2. 17.124 || FILIPIL | 2.17 | filamin A
interacting protein 1-like | 0.564700768 | 2.17.126 || FKBP5 | 2.17 | FK506 binding protein 5 |
1.68023215 | 2.17.127 || FNBPIL | 2.17 | formin binding protein 1 like | 1.739539906 | 2.17.124 ||
FRMD6 | 2.17 | FERM domain containing 6 | 0.813775409 | 2.17.127 || FRS2 | 2.17 | fibroblast
growth factor receptor substrate 2 | 0.497605461 | 2.17.125 || FRY | 2.17 | FRY microtubule binding
protein | 1.374563277 | 2.17.124 || FZD6 | 2.17 | frizzled class receptor 6 | 2.143549057 | 2.17.124 ||
GAB1 | 2.17 | GRB2 associated binding protein 1 | 0.894078708 | 2.17.125 || GABPA | 2.17 | GA
binding protein transcription factor alpha subunit | 0.252598746 | 2.17.124 || GCNT1 | 2.17 |
glucosaminyl (N-acetyl) transferase 1, core 2 | 0.256167146 | 2.17.125 || GJA1 | 2.17 | gap junction
protein alpha 1 | 0.608554029 | 2.17.127 || GJA5 | 2.17 | gap junction protein alpha 5 | 0.291113658
| 2.17.125 || GJC1 | 2.17 | gap junction protein gamma 1 | 1.409491655 | 2.17.126 || GLUL | 2.17 |
glutamate-ammonia ligase | 0.274341822 | 2.17.127 || GNG11 | 2.17 | G protein subunit gamma 11 |
0.647739649 | 2.17.125 || GPM6B | 2.17 | glycoprotein M6B | 1.430720582 | 2.17.127 || GUCY1A3
| 2.17 | guanylate cyclase 1, soluble, alpha 3 | 1.97743888 | 2.17.124 || GUCY1B3 | 2.17 | guanylate
cyclase 1, soluble, beta 3 | 0.787217465 | 2.17.125 || HACE1 | 2.17 | HECT domain and ankyrin
repeat containing E3 ubiquitin protein ligase 1 | 1.264601713 | 2.17.124 || HEPH | 2.17 | hephaestin |
0.811394897 | 2.17.125 || HHEX | 2.17 | hematopoietically expressed homeobox | 0.554651618 |
2.17.126 || HOXB3 | 2.17 | homeobox B3 | 0.361887424 | 2.17.125 || HS3ST1 | 2.17 | heparan
sulfate-glucosamine 3-sulfotransferase 1 | 1.093853563 | 2.17.124 || HTRA3 | 2.17 | HtrA serine
peptidase 3 | 0.393260739 | 2.17.125 || ICA1L | 2.17 | islet cell autoantigen 1 like | 0.29295291 |
2.17.125 || ID1 | 2.17 | inhibitor of DNA binding 1, HLH protein | 1.134172555 | 2.17.124 || IER5L |
2.17 | immediate early response 5-like | 0.315954629 | 2.17.127 || IFI6 | 2.17 | interferon, alpha-
inducible protein 6 | 1.122576884 | 2.17.124 || IKBIP | 2.17 | IKBKB interacting protein |
0.460792882 | 2.17.127 || IL17RD | 2.17 | interleukin 17 receptor D | 2.247920877 | 2.17.124 || IL33
| 2.17 | interleukin 33 | 3.774111262 | 2.17.124 || INPP5F | 2.17 | inositol polyphosphate-5-
phosphatase F | 0.28349311 | 2.17.124 || ITGA1 | 2.17 | integrin subunit alpha 1 | 0.896671188 |
2.17.124 || ITGAM | 2.17 | integrin subunit alpha M | 0.815666746 | 2.17.124 || ITGB1BP1 | 2.17 |
integrin subunit beta 1 binding protein 1 | 0.297177921 | 2.17.125 || JAG1 | 2.17 | jagged 1 |
0.713930058 | 2.17.124 || JAG2 | 2.17 | jagged 2 | 0.510481438 | 2.17.126 || JAM2 | 2.17 | junctional
adhesion molecule 2 | 1.376858038 | 2.17.124 || JAZF1 | 2.17 | JAZF zinc finger 1 | 0.225918822 |
2.17.125 || KATNAL1 | 2.17 | katanin p60 subunit A like 1 | 0.60518728 | 2.17.125 || KCNJ8 | 2.17 |
potassium voltage-gated channel subfamily J member 8 | 0.817309587 | 2.17.125 || KCTD15 | 2.17 |
potassium channel tetramerization domain containing 15 | 1.396051842 | 2.17.125| | KCTD9 | 2.17 |
potassium channel tetramerization domain containing 9 | 0.263593011 | 2.17.124 || KDELC1 | 2.17 |
KDEL motif containing 1 | 0.683934832 | 2.17.127 || KDR | 2.17 | kinase insert domain receptor |
1.052951732 | 2.17.124 || KLHL7 | 2.17 | kelch like family member 7 | 0.265123513 | 2.17.125 ||
LAMA4 | 2.17 | laminin subunit alpha 4 | 0.70683101 | 2.17.124 || LAMC1 | 2.17 | laminin subunit
gamma 1 | 0.292868302 | 2.17.125 || LARS2 | 2.17 | leucyl-tRNA synthetase 2 | 0.344724682 |
2.17.125 || LATS1 | 2.17 | large tumor suppressor kinase 1 | 0.294057251 | 2.17.124 || LBH | 2.17 |
limb bud and heart development | 0.484850762 | 2.17.127 || LDB2 | 2.17 | LIM domain binding 2 |
1.683616689 | 2.17.125 || LEF1 | 2.17 | lymphoid enhancer binding factor 1 | 1.169334617 | - ||
LHX6 | 2.17 | LIM homeobox 6 | 1.178086198 | 2.17.125 || LOC730102 | 2.17 | quinone
oxidoreductase-like protein 2 pseudogene | 0.356612157 | 2.17.125 || LRRC32 | 2.17 | leucine rich
repeat containing 32 | 0.706824339 | 2.17.124 || LRRN3 | 2.17 | leucine rich repeat neuronal 3 |
1.626717762 | 2.17.125 || LY86 | 2.17 | lymphocyte antigen 86 | 0.440570375 | 2.17.125 || MALL |
2.17 | mal, T-cell differentiation protein-like | 0.585499746 | 2.17.127 || MAP3K11 | 2.17 | mitogen-
activated protein kinase kinase kinase 11 | 0.239659423 | 2.17.126 || MAP4K4 | 2.17 | mitogen-
activated protein kinase kinase kinase kinase 4 | 0.599566721 | 2.17.125 || MBTD1 | 2.17 | mbt
domain containing 1 | 0.216931757 | 2.17.125 || MECOM | 2.17 | MDS1 and EVIL complex locus |
1.723154479 | 2.17.124 || MEGF9 | 2.17 | multiple EGF like domains 9 | 0.281345659 | 2.17.126 ||
MMRN2 | 2.17 | multimerin 2 | 0.723845587 | 2.17.125 || MPDZ | 2.17 | multiple PDZ domain
crumbs cell polarity complex component | 0.834525641 | 2.17.124 || MRPL1 | 2.17 | mitochondrial
ribosomal protein L1 | 0.416587495 | 2.17.124 || MUC1 | 2.17 | mucin 1, cell surface associated |
0.315899817 | 2.17.124 || MYCT1 | 2.17 | myc target 1 | 0.725785281 | 2.17.125 || MYH10 | 2.17 |
myosin, heavy chain 10, non-muscle | 1.164370368 | 2.17.125 || MYH9 | 2.17 | myosin, heavy chain
9, non-muscle | 0.319845011 | 2.17.126 || MYL9 | 2.17 | myosin light chain 9 | 0.775610304 |
2.17.125 || MYO9A | 2.17 | myosin IXA | 0.427453096 | 2.17.125 || NAB1 | 2.17 | NGFI-A binding
protein 1 | 0.226484788 | 2.17.124 || NEURLIB | 2.17 | neuralized E3 ubiquitin protein ligase 1B |
0.887200485 | 2.17.125 || NID1 | 2.17 | nidogen 1 | 1.492436869 | 2.17.124 || NID2 | 2.17 | nidogen
2 | 0.760702668 | 2.17.127 || NOV | 2.17 | nephroblastoma overexpressed | 2.844005169 | 2.17.124 ||
NR2F2 | 2.17 | nuclear receptor subfamily 2 group F member 2 | 0.742757478 | 2.17.124 || NRN1 |
2.17 | neuritin 1 | 2.075579212 | 2.17.124 || NUAK1 | 2.17 | NUAK family kinase 1 | 0.93337636 |
2.17.125 || NUDT7 | 2.17 | nudix hydrolase 7 | 0.501954265 | 2.17.125 || NUP35 | 2.17 | nucleoporin
35kDa | 0.267511533 | 2.17.124 || ODF2 | 2.17 | outer dense fiber of sperm tails 2 | 0.241298677 |
2.17.124 || OLFML2A | 2.17 | olfactomedin like 2A | 1.226393628 | 2.17.125 || PDE5A | 2.17 |
phosphodiesterase 5A | 1.833210985 | 2.17.124 || PI15 | 2.17 | peptidase inhibitor 15 | 0.982996573 |
2.17.124 || PIBF1 | 2.17 | progesterone immunomodulatory binding factor 1 | 0.32317475 | 2.17.124
|| PKDCC | 2.17 | protein kinase domain containing, cytoplasmic | 1.548070767 | 2.17.125 || PKP4 |
2.17 | plakophilin 4 | 1.099430083 | 2.17.124 || PLA2G7 | 2.17 | phospholipase A2 group VII |
0.382480958 | 2.17.126 || PLAU | 2.17 | plasminogen activator, urokinase | 0.404867694 | 2.17.127
|| PLCL1 | 2.17 | phospholipase C like 1 | 0.308847875 | 2.17.126 || PLEK2 | 2.17 | pleckstrin 2 |
0.345893639 | 2.17.126 || PLPPR4 | 2.17 | phospholipid phosphatase related 4 | 1.825862147 |
2.17.125 || PLVAP | 2.17 | plasmalemma vesicle associated protein | 0.570043008 | 2.17.126 ||
PODN | 2.17 | podocan | 0.58543331 | 2.17.125 || PRDX2 | 2.17 | peroxiredoxin 2 | 0.263026189 |
2.17.124 || PREX2 | 2.17 | phosphatidylinositol-3,4,5-trisphosphate-dependent Rac exchange factor
2 | 1.973934179 | 2.17.124 || PRKAG2 | 2.17 | protein kinase AMP-activated non-catalytic subunit
gamma 2 | 0.229869396 | - || PRKCDBP | 2.17 | protein kinase C delta binding protein |
0.691940304 | 2.17.127 || PRKD1 | 2.17 | protein kinase D1 | 0.659899578 | 2.17.125 || PTGR1 |
2.17 | prostaglandin reductase 1 | 0.280966942 | 2.17.124 || PTK2 | 2.17 | protein tyrosine kinase 2 |
0.261036342 | 2.17.125 || PTPN21 | 2.17 | protein tyrosine phosphatase, non-receptor type 21 |
0.928169001 | 2.17.125 || PTPRM | 2.17 | protein tyrosine phosphatase, receptor type M |
0.255088825 | 2.17.125 || PWWP2A | 2.17 | PWWP domain containing 2A | 0.28773633 | 2.17.124
|| PXN | 2.17 | paxillin | 0.249626232 | 2.17.126 || RAMP3 | 2.17 | receptor (G protein-coupled)
activity modifying protein 3 | 0.84866751 | 2.17.124 || RARA | 2.17 | retinoic acid receptor alpha |
6.344036067 | 2.17.125 || RASL12 | 2.17 | RAS like family 12 | 0.470244108 | 2.17.125 || RASSF3 |
2.17 | Ras association domain family member 3 | 0.255685815 | 2.17.126 || RFTN1 | 2.17 | raftlin,
lipid raft linker 1 | 0.541508935 | 2.17.126 || RHOJ | 2.17 | ras homolog family member J |
2.003469568 | 2.17.124 || RNF123 | 2.17 | ring finger protein 123 | 0.241512255 | 2.17.124 ||
RNF144A | 2.17 | ring finger protein 144A | 0.709145952 | 2.17.124 || ROBO4 | 2.17 | roundabout
guidance receptor 4 | 0.718387723 | 2.17.125 || ROCK2 | 2.17 | Rho associated coiled-coil
containing protein kinase 2 | 0.714350491 | 2.17.125 || S100A16 | 2.17 | S100 calcium binding
protein A16 | 0.390001119 | 2.17.124 || S1PR1 | 2.17 | sphingosine-1-phosphate receptor 1 |
0.332036442 | 2.17.125 || SCN4B | 2.17 | sodium voltage-gated channel beta subunit 4 |
1.674532567 | 2.17.125 || SDPR | 2.17 | serum deprivation response | 3.070893994 | 2.17.125 ||
SERPINH1 | 2.17 | serpin peptidase inhibitor, clade H (heat shock protein 47), member 1, (collagen
binding protein 1) | 0.622630995 | - || SFRP4 | 2.17 | secreted frizzled-related protein 4 |
3.262301646 | 2.17.124 || SH2D3C | 2.17 | SH2 domain containing 3C | 0.404744321 | 2.17.126 ||
SH3BP4 | 2.17 | SH3-domain binding protein 4 | 0.656715251 | 2.17.125 || SHANK3 | 2.17 | SH3
and multiple ankyrin repeat domains 3 | 1.371147921 | 2.17.125 || SHE | 2.17 | Src homology 2
domain containing E | 0.522594512 | 2.17.125 || SIDT2 | 2.17 | SID1 transmembrane family
member 2 | 0.290703187 | - || SKP2 | 2.17 | S-phase kinase-associated protein 2, E3 ubiquitin
protein ligase | 0.505014297 | 2.17.125 || SLC12A2 | 2.17 | solute carrier family 12 member 2 |
0.614484188 | 2.17.124 || SLC1A3 | 2.17 | solute carrier family 1 member 3 | 1.689860068 |
2.17.127 || SLC30A4 | 2.17 | solute carrier family 30 member 4 | 0.215053029 | 2.17.124 ||
SLC39A10 | 2.17 | solute carrier family 39 member 10 | 0.266715622 | 2.17.124 || SLIT3 | 2.17 | slit
guidance ligand 3 | 0.70833372 | 2.17.125 || SMAD1 | 2.17 | SMAD family member 1 |
1.235149314 | 2.17.124 || SMIM3 | 2.17 | small integral membrane protein 3 | 0.330690153 |
2.17.127 || SMU1 | 2.17 | smu-1 suppressor of mec-8 and unc-52 homolog (C. elegans) | 0.22452513
| - || SNAI2 | 2.17 | snail family zinc finger 2 | 0.818480827 | - || SNCAIP | 2.17 | synuclein alpha
interacting protein | 0.588192354 | 2.17.124 || SNRK | 2.17 | SNF related kinase | 0.286185619 |
2.17.125 || SOCS2 | 2.17 | suppressor of cytokine signaling 2 | 0.913068369 | 2.17.124 || SOX4 |
2.17 | SRY-box 4 | 0.936367705 | 2.17.127 || SOX7 | 2.17 | SRY-box 7 | 1.873947217 | 2.17.125 ||
SPATS2 | 2.17 | spermatogenesis associated serine rich 2 | 0.275087211 | - || SPNS2 | 2.17 | spinster
homolog 2 (Drosophila) | 0.36332731 | 2.17.125 || STARD9 | 2.17 | StAR related lipid transfer
domain containing 9 | 0.67986022 | 2.17.125 || STON2 | 2.17 | stonin 2 | 0.851091766 | 2.17.124 ||
SYNE2 | 2.17 | spectrin repeat containing, nuclear envelope 2 | 1.976751419 | 2.17.126 || TAF12 |
2.17 | TATA-box binding protein associated factor 12 | 0.22949498 | 2.17.124 || TAGLN | 2.17 |
transgelin | 1.219161942 | 2.17.124 || TANC1 | 2.17 | tetratricopeptide repeat, ankyrin repeat and
coiled-coil containing 1 | 1.727561303 | 2.17.124 || TEK | 2.17 | TEK receptor tyrosine kinase |
1.433203869 | 2.17.124 || TGFB3 | 2.17 | transforming growth factor beta 3 | 0.504305292 |
2.17.127 || THSD7A | 2.17 | thrombospondin type 1 domain containing 7A | 2.484067083 | 2.17.125
|| THY1 | 2.17 | Thy-1 cell surface antigen | 0.688637056 | - || TM4SF1 | 2.17 | transmembrane 4 L
six family member 1 | 0.79382655 | 2.17.124 || TM4SF18 | 2.17 | transmembrane 4 L six family
member 18 | 2.53876737 | 2.17.124 || TMEM204 | 2.17 | transmembrane protein 204 | 0.484166564 |
2.17.125 || TMEM47 | 2.17 | transmembrane protein 47 | 0.970410186 | 2.17.124 || TPM1 | 2.17 |
tropomyosin 1 (alpha) | 0.498805397 | 2.17.124 || TSPAN12 | 2.17 | tetraspanin 12 | 2.208235108 |
2.17.124 || TUSC3 | 2.17 | tumor suppressor candidate 3 | 0.861076537 | 2.17.127 || TWSG1 | 2.17 |
twisted gastrulation BMP signaling modulator 1 | 0.258197131 | 2.17.127 || VWA5A | 2.17 | von
Willebrand factor A domain containing 5A | 0.491093621 | 2.17.127 || XRCC2 | 2.17 | X-ray repair
complementing defective repair in Chinese hamster cells 2 | 0.34576168 | 2.17.126 || ZBTB46 | 2.17
| zinc finger and BTB domain containing 46 | 0.267531395 | 2.17.125 || ZCCHC2 | 2.17 | zinc finger
CCHC-type containing 2 | 2.080932972 | 2.17.127 || ZMYM4 | 2.17 | zinc finger MYM-type
containing 4 | 0.227299065 | 2.17.125 || ZNF227 | 2.17 | zinc finger protein 227 | 0.286384307 |
2.17.125 || ZNF529 | 2.17 | zinc finger protein 529 | 0.358239722 | 2.17.124 || ZNF614 | 2.17 | zinc
finger protein 614 | 0.266891515 | 2.17.124 || ZNF808 | 2.17 | zinc finger protein 808 | 0.312485463
| 2.17.127 | ZNHIT6 | 2.17 | zinc finger HIT-type containing 6 | 0.291553481 | 2.17.124 || AKNA |
2.18.129 | AT-hook transcription factor | 0.464370735 | 2.18.129 || BIRC3 | 2.18.129 | baculoviral
IAP repeat containing 3 | 0.665883746 | 2.18.129 || CD2 | 2.18.129 | CD2 molecule | 1.920481085 |
2.18.129 || CD3D | 2.18.129 | CD3d molecule | 2.267328959 | 2.18.129 || CD3G | 2.18.129 | CD3g
molecule | 1.1898177 | 2.18.129 || CD8A | 2.18.129 | CD8a molecule | 1.502794773 | 2.18.129 ||
CST7 | 2.18.129 | cystatin F | 0.257916524 | 2.18.129 || CTSW | 2.18.129 | cathepsin W |
0.668527574 | 2.18.129 || CXCL13 | 2.18.129 | C-X-C motif chemokine ligand 13 | 7.675943343 |
2.18.129 || DENND2D | 2.18.129 | DENN domain containing 2D | 0.410314385 | 2.18.129 ||
EOMES | 2.18.129 | eomesodermin | 1.215836207 | 2.18.129 || GCLC | 2.18.129 | glutamate-
cysteine ligase catalytic subunit | 0.232367024 | 2.18.129 || GZMA | 2.18.129 | granzyme A |
4.131331048 | 2.18.129 || GZMH | 2.18.129 | granzyme H | 1.344984481 | 2.18.129 || GZMK |
2.18.129 | granzyme K | 4.225613474 | 2.18.129 || IL32 | 2.18.129 | interleukin 32 | 1.777840703 |
2.18.129 || ITK | 2.18.129 | IL2 inducible T-cell kinase | 1.353024592 | 2.18.129 || KIAA1551 |
2.18.129 | KIAA1551 | 0.365408529 | 2.18.129 || KLRB1 | 2.18.129 | killer cell lectin like receptor
B1 | 0.751935713 | 2.18.129 || LCK | 2.18.129 | LCK proto-oncogene, Src family tyrosine kinase |
2.104403831 | 2.18.129 || NKG7 | 2.18.129 | natural killer cell granule protein 7 | 1.708688625 |
2.18.129 || NLRC3 | 2.18.129 | NLR family, CARD domain containing 3 | 0.216441871 | 2.18.129 ||
PDCD4 | 2.18.129 | programmed cell death 4 (neoplastic transformation inhibitor) | 0.325133438 |
2.18.129 || PRF1 | 2.18.129 | perforin 1 | 1.331921421 | 2.18.129 || PTPRCAP | 2.18.129 | protein
tyrosine phosphatase, receptor type C associated protein | 0.611743348 | 2.18.129 || RASGRP1 |
2.18.129 | RAS guanyl releasing protein 1 | 1.931153541 | 2.18.129 || SH2DIA | 2.18.129 | SH2
domain containing 1A | 0.653395578 | 2.18.129 || STAMBPL1 | 2.18.129 | STAM binding protein
like 1 | 0.356935406 | 2.18.129 || TOX | 2.18.129 | thymocyte selection associated high mobility
group box | 0.245434058 | 2.18.129 || TRAF3IP3 | 2.18.129 | TRAF3 interacting protein 3 |
0.769992424 | 2.18.129 || TRAT1 | 2.18.129 | T cell receptor associated transmembrane adaptor 1 |
0.894694549 | 2.18.129 || TSPAN13 | 2.18.129 | tetraspanin 13 | 0.782661139 | 2.18.129 || VNN1 |
2.18.129 | vanin 1 | 1.310387591 | 2.18.129 || XCL1 | 2.18.129 | X-C motif chemokine ligand 1 |
0.455387609 | 2.18.129 || ADAR | 2.18.130 | adenosine deaminase, RNA-specific | 0.225307616 |
2.18.130 || APOE | 2.18.130 | apolipoprotein E | 0.991942596 | 2.18.130 || APOL1 | 2.18.130 |
apolipoprotein L1 | 0.647504829 | 2.18.130 || Clorf53 | 2.18.130 | chromosome 1 open reading
frame 53 | 0.526630676 | 2.18.130 || CD6 | 2.18.130 | CD6 molecule | 0.374716271 | 2.18.130 ||
CLEC4A | 2.18.130 | C-type lectin domain family 4 member A | 0.502901431 | 2.18.130 || CMPK2 |
2.18.130 | cytidine/uridine monophosphate kinase 2 | 1.85702603 | 2.18.130 || CTDSPL2 | 2.18.130 |
CTD small phosphatase like 2 | 0.268585085 | 2.18.130 || DDX60 | 2.18.130 | DEXD/H-box
helicase 60 | 0.346977964 | 2.18.130 || EIF2AK2 | 2.18.130 | eukaryotic translation initiation factor
2 alpha kinase 2 | 0.318282855 | 2.18.130 || FCGR3B | 2.18.130 | Fc fragment of IgG receptor IIIb |
0.9155816 | 2.18.130 || FGL2 | 2.18.130 | fibrinogen like 2 | 0.584297365 | 2.18.130 || FMO3 |
2.18.130 | flavin containing monooxygenase 3 | 1.156549149 | 2.18.130 || FST | 2.18.130 | follistatin
| 0.540062872 | 2.18.130 || HPSE | 2.18.130 | heparanase | 1.247455214 | 2.18.130 || IFI35 | 2.18.130
| interferon induced protein 35 | 0.850518409 | 2.18. 130 || IFI44 | 2.18.130 | interferon induced
protein 44 | 0.813721828 | 2.18.130 || IFI44L | 2.18.130 | interferon induced protein 44 like |
1.821139894 | 2.18.130 || IFIH1 | 2.18.130 | interferon induced, with helicase C domain 1 |
0.554506761 | 2.18.130 || IFIT1 | 2.18.130 | interferon induced protein with tetratricopeptide repeats
1 | 0.916194528 | 2.18.130 || IFIT2 | 2.18.130 | interferon induced protein with tetratricopeptide
repeats 2 | 0.79521356 | 2.18.130 || IFIT3 | 2.18.130 | interferon induced protein with
tetratricopeptide repeats 3 | 0.789995391 | 2.18.130 || INSIG1 | 2.18.130 | insulin induced gene 1 |
0.28016302 | 2.18.130 || IRF9 | 2.18.130 | interferon regulatory factor 9 | 0.254031035 | 2.18.130 ||
ISG15 | 2.18.130 | ISG15 ubiquitin-like modifier | 0.999772267 | 2.18.130 || LGALS3BP | 2.18.130
| lectin, galactoside-binding, soluble, 3 binding protein | 0.386352906 | 2.18.130 || LXN | 2.18.130 |
latexin | 0.798649782 | 2.18.130 || LYSMD2 | 2.18.130 | LysM domain containing 2 | 0.318579984 |
2.18.130 || MICAL1 | 2.18.130 | microtubule associated monooxygenase, calponin and LIM domain
containing 1 | 0.31709165 | 2.18.130 || MX1 | 2.18.130 | MX dynamin like GTPase 1 | 1.356763919
| 2.18.130 | MX2 | 2.18.130 | MX dynamin like GTPase 2 | 1.279741767 | 2.18.130 || NMI |
2.18.130 | N-myc and STAT interactor | 0.445756082 | 2.18.130 || OAS1 | 2.18.130 | 2′-5′-
oligoadenylate synthetase 1 | 1.295257967 | 2.18.130 || OAS2 | 2.18.130 | 2′-5′-oligoadenylate
synthetase 2 | 1.52539947 | 2.18.130 || OAS3 | 2.18.130 | 2′-5′-oligoadenylate synthetase 3 |
1.089135973 | 2.18.130 || OASL | 2.18.130 | 2′-5′-oligoadenylate synthetase like | 0.562245942 |
2.18.130 || PARP12 | 2.18.130 | poly(ADP-ribose) polymerase family member 12 | 0.748927974 |
2.18.130 || PARP14 | 2.18.130 | poly(ADP-ribose) polymerase family member 14 | 0.580266692 |
2.18.130 || PARP9 | 2.18.130 | poly(ADP-ribose) polymerase family member 9 | 0.3652565 |
2.18.130 || PRRG4 | 2.18.130 | proline rich Gla (G-carboxyglutamic acid) 4 (transmembrane) |
0.35055221 | 2.18.130 || RSAD2 | 2.18.130 | radical S-adenosyl methionine domain containing 2 |
1.617422099 | 2.18.130 || S100A8 | 2.18.130 | S100 calcium binding protein A8 | 1.214556196 |
2.18.130 || SAMD9L | 2.18.130 | sterile alpha motif domain containing 9-like | 0.545082576 |
2.18.130 || SDC4 | 2.18.130 | syndecan 4 | 0.483005798 | 2.18.130 | STAT2 | 2.18.130 | signal
transducer and activator of transcription 2 | 0.219550065 | 2.18.130 || STOX2 | 2.18.130 | storkhead
box 2 | 0.371157973 | 2.18.130 || SUN2 | 2.18.130 | Sad1 and UNC84 domain containing 2 |
0.300036584 | 2.18.130 || TRDMT1 | 2.18.130 | tRNA aspartic acid methyltransferase 1 |
0.285733098 | 2.18.130 || VAMP5 | 2.18.130 | vesicle associated membrane protein 5 | 0.480879097
| 2.18.130 || VRK2 | 2.18.130 | vaccinia related kinase 2 | 0.219343149 | 2.18.130 || XAF1 | 2.18.130
| XIAP associated factor 1 | 0.528186189 | 2.18.130 || AMER1 | 2.19 | APC membrane recruitment
protein 1 | 0.50274814 | 2.19.133 || ANKRD36BP2 | 2.19 | ankyrin repeat domain 36B pseudogene
2 | 0.525829363 | 2.19.133 || ANOS1 | 2.19 | anosmin 1 | 0.980796077 | 2.19.132 || APCDDIL | 2.19
| adenomatosis polyposis coli down-regulated 1-like | 0.222203679 | 2.19.132 || BHLHE22 | 2.19 |
basic helix-loop-helix family member e22 | 0.278392269 | 2.19.132 || BMS1P20 | 2.19 | BMS1,
ribosome biogenesis factor pseudogene 20 | 5.907840779 | 2.19.133 || BTG2 | 2.19 | BTG family
member 2 | 0.403392154 | 2.19.133 || C18orf54 | 2.19 | chromosome 18 open reading frame 54 |
0.46889736 | 2.19.133 || CD27 | 2.19 | CD27 molecule | 1.56949754 | 2.19.133 || CD79A | 2.19 |
CD79a molecule | 2.879891667 | 2.19.133 || CD79B | 2.19 | CD79b molecule | 0.759518551 |
2.19.133 || CHRNG | 2.19 | cholinergic receptor nicotinic gamma subunit | 0.308008286 | 2.19.133 ||
COL11A1 | 2.19 | collagen type XI alpha 1 | 4.086943037 | 2.19.132 || COL25A1 | 2.19 | collagen
type XXV alpha 1 | 0.226206641 | 2.19.133 || CPNE5 | 2.19 | copine 5 | 1.28299047 | 2.19.133 ||
CRELD2 | 2.19 | cysteine rich with EGF like domains 2 | 0.249185191 | 2.19.133 || EAF2 | 2.19 |
ELL associated factor 2 | 1.119908659 | 2.19.133 || EPB41 | 2.19 | erythrocyte membrane protein
band 4.1 | 0.250479543 | 2.19.132 || ERAP1 | 2.19 | endoplasmic reticulum aminopeptidase 1 |
0.839914071 | 2.19.132 || ERMP1 | 2.19 | endoplasmic reticulum metallopeptidase 1 | 0.260101199 |
2.19.132 || FAM46C | 2.19 | family with sequence similarity 46 member C | 2.638930709 | 2.19.133
|| FCMR | 2.19 | Fc fragment of IgM receptor | 2.283307814 | 2.19.132 || FCRL5 | 2.19 | Fc receptor
like 5 | 3.627434799 | 2.19.132 || FKBP11 | 2.19 | FK506 binding protein 11 | 0.633428957 |
2.19.132 || FRMD4B | 2.19 | FERM domain containing 4B | 0.216226296 | 2.19.133 || GCLM | 2.19
| glutamate-cysteine ligase modifier subunit | 0.266062766 | 2.19.133 || GPR26 | 2.19 | G protein-
coupled receptor 26 | 0.214475114 | 2.19.133 || GUSBP11 | 2.19 | glucuronidase, beta pseudogene
11 | 3.13291442 | 2.19.132 || HERPUD1 | 2.19 | homocysteine-inducible, endoplasmic reticulum
stress-inducible, ubiquitin-like domain member 1 | 0.282531445 | 2.19.133 || IKZF3 | 2.19 |
IKAROS family zinc finger 3 | 0.301579275 | 2.19.133 || ING2 | 2.19 | inhibitor of growth family
member 2 | 0.317272413 | 2.19.133 || ISG20 | 2.19 | interferon stimulated exonuclease gene 20kDa |
2.041973791 | 2.19.133 || ITM2C | 2.19 | integral membrane protein 2C | 1.126536183 | 2.19.133 ||
JCHAIN | 2.19 | joining chain of multimeric IgA and IgM | 5.896710789 | 2.19.132 || KNTC1 | 2.19
| kinetochore associated 1 | 0.358488124 | 2.19.133 || KRT222 | 2.19 | keratin 222 | 1.07253478 |
2.19.132 || LACC1 | 2.19 | laccase domain containing 1 | 0.374901618 | 2.19.133 || LAX1 | 2.19 |
lymphocyte transmembrane adaptor 1 | 0.782768797 | 2.19.132 || LGALS8 | 2.19 | lectin,
galactoside-binding, soluble, 8 | 0.338306562 | 2.19.133 || LGMN | 2.19 | legumain | 0.233818531 |
2.19.132 || MEI1 | 2.19 | meiotic double-stranded break formation protein 1 | 0.80281807 | 2.19.133
|| MFHAS1 | 2.19 | malignant fibrous histiocytoma amplified sequence 1 | 0.261979953 | 2.19.133 ||
MIAT | 2.19 | myocardial infarction associated transcript (non-protein coding) | 0.295785352 |
2.19.133 || MS4A1 | 2.19 | membrane spanning 4-domains Al | 5.929678189 | 2.19.132 || MZB1 |
2.19 | marginal zone B and B1 cell specific protein | 5.087395139 | 2.19.132 || NABP2 | 2.19 |
nucleic acid binding protein 2 | 0.236296146 | 2.19.133 || NELL2 | 2.19 | neural EGFL like 2 |
0.387771765 | 2.19.132 || NRP2 | 2.19 | neuropilin 2 | 0.418605035 | 2.19.133 || NTN1 | 2.19 | netrin
1 | 0.977358167 | 2.19.132 || OMD | 2.19 | osteomodulin | 2.010523976 | 2.19.132 || P2RX5 | 2.19 |
purinergic receptor P2X 5 | 0.800455048 | 2.19.133 || PDCDILG2 | 2.19 | programmed cell death 1
ligand 2 | 0.225415117 | 2.19.133 || PDK1 | 2.19 | pyruvate dehydrogenase kinase 1 | 1.265409784 |
2.19.133 || PIM2 | 2.19 | Pim-2 proto-oncogene, serine/threonine kinase | 2.54261936 | 2.19.132 ||
PLPP5 | 2.19 | phospholipid phosphatase 5 | 0.306185429 | 2.19.132 || POU2AF1 | 2.19 | POU class
2 associating factor 1 | 6.127952622 | 2.19.132 || PRDM1 | 2.19 | PR domain 1 | 1.057510938 |
2.19.132 || PRDX4 | 2.19 | peroxiredoxin 4 | 0.301002066 | 2.19.132 || RNF141 | 2.19 | ring finger
protein 141 | 0.233401383 | 2.19.132 || SDC1 | 2.19 | syndecan 1 | 2.630474379 | 2.19.133 ||
SEC11C | 2.19 | SEC11 homolog C, signal peptidase complex subunit | 0.90082139 | 2.19.133 ||
SERPINE2 | 2.19 | serpin peptidase inhibitor, clade E (nexin, plasminogen activator inhibitor type
1), member 2 | 1.567647357 | 2.19.132 || SHROOM1 | 2.19 | shroom family member 1 |
0.278403439 | 2.19.133 || SLC25A43 | 2.19 | solute carrier family 25 member 43 | 0.369260817 |
2.19.133 || SLC38A1 | 2.19 | solute carrier family 38 member 1 | 1.024186126 | 2.19.132 || SLC7A5
| 2.19 | solute carrier family 7 member 5 | 0.827271478 | 2.19.133 || SNX25 | 2.19 | sorting nexin 25
0.263921575 | 2.19.132 || SPAG4 | 2.19 | sperm associated antigen 4 | 2.366853485 | 2.19.133 ||
SPOCK2 | 2.19 | sparc/osteonectin, cwcv and kazal-like domains proteoglycan (testican) 2 |
0.341752292 | 2.19.133 || SSR4 | 2.19 | signal sequence receptor, delta | 0.458774358 | 2.19.133 ||
ST6GAL1 | 2.19 | ST6 beta-galactosamide alpha-2,6-sialyltranferase 1 | 0.582754683 | 2.19.133 ||
SYTL1 | 2.19 | synaptotagmin like 1 | 0.921967393 | 2.19.133 || TDRD3 | 2.19 | tudor domain
containing 3 | 0.459567248 | 2.19.133 || TIFA | 2.19 | TRAF interacting protein with forkhead
associated domain | 0.487811773 | 2.19.132 || TNFRSF17 | 2.19 | tumor necrosis factor receptor
superfamily member 17 | 3.462596721 | 2.19.133 || TP53INP1 | 2.19 | tumor protein p53 inducible
nuclear protein 1 | 0.308472309 | 2.19.132 || TPD52 | 2.19 | tumor protein D52 | 2.035248091 |
2.19.132 || TTC7B | 2.19 | tetratricopeptide repeat domain 7B | 0.361215418 | 2.19.133 || TXNDC15
| 2.19 | thioredoxin domain containing 15 | 0.254461953 | 2.19.133 || UHRF1BP1 | 2.19 | UHRF1
binding protein 1 | 0.402231232 | 2.19.132 || XBP1 | 2.19 | X-box binding protein 1 | 0.763304853 |
2.19.132 || XRRA1 | 2.19 | X-ray radiation resistance associated 1 | 0.793846928 | 2.19.133 || ZFP64
| 2.19 | ZFP64 zinc finger protein | 0.236269082 | 2.19.133 || CPSF2 | 2.20.134 | cleavage and
polyadenylation specific factor 2 | 0.312514552 | 2.20.134 || CTNNBIP1 | 2.20.134 | catenin beta
interacting protein 1 | 0.364519822 | 2.20.134 || CYP1A2 | 2.20.134 | cytochrome P450 family 1
subfamily A member 2 | 0.309345787 | 2.20.134 || FAM109B | 2.20.134 | family with sequence
similarity 109 member B | 0.580511341 | 2.20.134 || GMEB1 | 2.20.134 | glucocorticoid modulatory
element binding protein 1 | 0.356125841 | 2.20.134 || HMG20B | 2.20.134 | high mobility group
20B | 0.295005592 | 2.20.134 || IRF5 | 2.20.134 | interferon regulatory factor 5 | 1.044864182 |
2.20.134 || ITGB3BP | 2.20.134 | integrin subunit beta 3 binding protein | 0.719615236 | 2.20.134 ||
LOC441666 | 2.20.134 | zinc finger protein 91 pseudogene | 1.441786817 | 2.20.134 || OPHN1 |
2.20.134 | oligophrenin 1 | 0.333288257 | 2.20.134 || PANX1 | 2.20.134 || pannexin 1 | 0.279004738 |
2.20.134 || RASEF | 2.20.134 | RAS and EF-hand domain containing | 0.39832375 | 2.20.134 ||
RECK | 2.20.134 | reversion inducing cysteine rich protein with kazal motifs | 0.252057572 |
2.20.134 || SCD5 | 2.20.134 | stearoyl-CoA desaturase 5 | 0.748681998 | 2.20.134 || SH3GL3 |
2.20.134 | SH3-domain GRB2-like 3 | 1.108918296 | 2.20.134 || SLC20A1 | 2.20.134 | solute carrier
family 20 member 1 | 0.31769957 | 2.20.134 || TIMM8A | 2.20.134 | translocase of inner
mitochondrial membrane 8 homolog A (yeast) | 0.263195296 | 2.20.134 || TMEM241 | 2.20.134 |
transmembrane protein 241 | 0.411636957 | 2.20.134 || TRIB2 | 2.20.134 | tribbles pseudokinase 2 |
0.281368198 | 2.20.134 || TRIM4 | 2.20.134 | tripartite motif containing 4 | 0.530234587 | 2.20.134 ||
TRIM47 | 2.20.134 | tripartite motif containing 47 | 0.328967302 | 2.20.134 || VPS37B | 2.20.134 |
VPS37B, ESCRT-I subunit | 0.293588562 | 2.20.134 || YPEL3 | 2.20.134 | yippee like 3 | 0.3094805
| 2.20.134 || ZNF595 | 2.20.134 | zinc finger protein 595 | 4.945063628 | 2.20.134 || ACSS3 | 2.5.57 |
acyl-CoA synthetase short-chain family member 3 | 1.256611482 | 2.5.57 || ADIRF | 2.5.57 |
adipogenesis regulatory factor | 2.358053196 | 2.5.57 || ADRA2A | 2.5.57 | adrenoceptor alpha 2A |
4.385344968 | 2.5.57 || AKRIC1 | 2.5.57 | aldo-keto reductase family 1, member C1 | 1.57756812 |
2.5.57 || AKR1C2 | 2.5.57 | aldo-keto reductase family 1, member C2 | 1.899316279 | 2.5.57 ||
ALDH6A1 | 2.5.57 | aldehyde dehydrogenase 6 family member Al | 0.503721404 | 2.5.57 || AR |
2.5.57 | androgen receptor | 1.230295387 | 2.5.57 || ARHGAP42 | 2.5.57 | Rho GTPase activating
protein 42 | 0.510328956 | 2.5.57 || ATP2B4 | 2.5.57 | ATPase plasma membrane Ca2+ transporting
4 | 0.312991447 | 2.5.57 || ATP9A | 2.5.57 | ATPase phospholipid transporting 9A (putative) |
1.483950078 | 2.5.57 || BDKRB2 | 2.5.57 | bradykinin receptor B2 | 0.566291259 | 2.5.57 || BOK |
2.5.57 | BCL2-related ovarian killer | 1.599041866 | 2.5.57 || C19orf12 | 2.5.57 | chromosome 19
open reading frame 12 | 0.378582294 | 2.5.57 || Clorf198 | 2.5.57 | chromosome 1 open reading
frame 198 | 0.320478948 | 2.5.57 || Clorf21 | 2.5.57 | chromosome 1 open reading frame 21 |
0.612533147 | 2.5.57 || CCDC69 | 2.5.57 | coiled-coil domain containing 69 | 0.585257644 | 2.5.57 ||
CD300LG | 2.5.57 | CD300 molecule like family member g | 2.343699976 | 2.5.57 || CDC42EP4 |
2.5.57 | CDC42 effector protein 4 | 0.490169413 | 2.5.57 || CHRDL1 | 2.5.57 | chordin-like 1 |
6.246501097 | 2.5.57 || CLMP | 2.5.57 | CXADR-like membrane protein | 0.852551641 | 2.5.57 ||
CYB5A | 2.5.57 | cytochrome b5 type A (microsomal) | 0.738641051 | 2.5.57 || DTX4 | 2.5.57 |
deltex 4, E3 ubiquitin ligase | 0.39616286 | 2.5.57 || EHBP1 | 2.5.57 | EH domain binding protein 1 |
0.938640122 | 2.5.57 || EHD2 | 2.5.57 | EH domain containing 2 | 0.65223393 | 2.5.57 || F11R |
2.5.57 | F11 receptor | 0.449768336 | 2.5.57 || FABP4 | 2.5.57 | fatty acid binding protein 4 |
9.948984309 | 2.5.57 || FAHD2A | 2.5.57 | fumarylacetoacetate hydrolase domain containing 2A |
0.397171336 | 2.5.57 || FAM213A | 2.5.57 | family with sequence similarity 213 member A |
2.244678642 | 2.5.57 || FERMT2 | 2.5.57 | fermitin family member 2 | 0.514762935 | 2.5.57 ||
FGFBP2 | 2.5.57 | fibroblast growth factor binding protein 2 | 5.210037915 | 2.5.57 || FZD4 | 2.5.57
| frizzled class receptor 4 | 1.516536159 | 2.5.57 || GHR | 2.5.57 | growth hormone receptor |
5.216950489 | 2.5.57 || GNAI1 | 2.5.57 | G protein subunit alpha il | 3.712385374 | 2.5.57 ||
GPATCH11 | 2.5.57 | G-patch domain containing 11 | 0.248577992 | 2.5.57 || HADH | 2.5.57 |
hydroxyacyl-CoA dehydrogenase | 0.973225336 | 2.5.57 || HSDL2 | 2.5.57 | hydroxysteroid
dehydrogenase like 2 | 1.035299237 | 2.5.57 || IGFBP6 | 2.5.57 | insulin like growth factor binding
protein 6 | 1.144522243 | 2.5.57 || IGSF21 | 2.5.57 | immunoglobin superfamily member 21 |
0.817858137 | 2.5.57 || INHBB | 2.5.57 | inhibin beta B | 1.565653343 | 2.5.57 || ITGA7 | 2.5.57 |
integrin subunit alpha 7 | 1.731401261 | 2.5.57 || ITIH5 | 2.5.57 | inter-alpha-trypsin inhibitor heavy
chain family member 5 | 3.462851228 | 2.5.57 || JADE1 | 2.5.57 | jade family PHD finger 1 |
0.796229967 | 2.5.57 | KAT2B | 2.5.57 | lysine acetyltransferase 2B | 0.246457883 | 2.5.57 ||
KLF15 | 2.5.57 | Kruppel-like factor 15 | 0.404555382 | 2.5.57 || KYNU | 2.5.57 | kynureninase |
1.07034307 | 2.5.57 || LGALS12 | 2.5.57 | lectin, galactoside-binding, soluble, 12 | 1.350671351 |
2.5.57 || LGR4 | 2.5.57 | leucine-rich repeat containing G protein-coupled receptor 4 | 1.311926721 |
2.5.57 || LIN7A | 2.5.57 | lin-7 homolog A, crumbs cell polarity complex component | 0.713127587 |
2.5.57 || LINC01003 | 2.5.57 | long intergenic non-protein coding RNA 1003 | 0.611201414 | 2.5.57
|| LYRM1 | 2.5.57 | LYR motif containing 1 | 0.641753021 | 2.5.57 || MARC2 | 2.5.57 |
mitochondrial amidoxime reducing component 2 | 0.539094609 | 2.5.57 || MAST4 | 2.5.57 |
microtubule associated serine/threonine kinase family member 4 | 1.119455151 | 2.5.57 || MCCC1 |
2.5.57 | methylcrotonoyl-CoA carboxylase 1 | 0.432055636 | 2.5.57 || MEST | 2.5.57 | mesoderm
specific transcript | 5.725357017 | 2.5.57 || MFAP5 | 2.5.57 | microfibrillar associated protein 5 |
4.485133533 | 2.5.57 || MGAT4A | 2.5.57 | mannosyl (alpha-1,3-)-glycoprotein beta-1,4-N-
acetylglucosaminyltransferase, isozyme A | 0.371351088 | 2.5.57 || MGLL | 2.5.57 | monoglyceride
lipase | 0.819650779 | 2.5.57 || MGST1 | 2.5.57 | microsomal glutathione S-transferase 1 |
3.86837584 | 2.5.57 || MME | 2.5.57 | membrane metallo-endopeptidase | 0.721228403 | 2.5.57 ||
MTURN | 2.5.57 | maturin, neural progenitor differentiation regulator homolog (Xenopus) |
2.881061114 | 2.5.57 || MUT | 2.5.57 | methylmalonyl-CoA mutase | 0.26506776 | 2.5.57 || NCOA1
| 2.5.57 | nuclear receptor coactivator 1 | 0.249938333 | 2.5.57 || NNAT | 2.5.57 | neuronatin |
0.723923091 | 2.5.57 || PALMD | 2.5.57 | palmdelphin | 1.312827119 | 2.5.57 || PCCA | 2.5.57 |
propionyl-CoA carboxylase alpha subunit | 0.290492087 | 2.5.57 || PCK1 | 2.5.57 |
phosphoenolpyruvate carboxykinase 1 | 9.173211642 | 2.5.57 || PDHX | 2.5.57 | pyruvate
dehydrogenase complex component X | 0.540566351 | 2.5.57 || PERP | 2.5.57 | PERP, TP53
apoptosis effector | 1.534320177 | 2.5.57 || PET117 | 2.5.57 | PET117 homolog | 0.304053751 |
2.5.57 || PEX11A | 2.5.57 | peroxisomal biogenesis factor 11 alpha | 0.707187052 | 2.5.57 ||
PHLDB2 | 2.5.57 | pleckstrin homology like domain family B member 2 | 0.629724474 | 2.5.57 ||
PIR | 2.5.57 | pirin | 0.962374479 | 2.5.57 || PJA1 | 2.5.57 | praja ring finger ubiquitin ligase 1 |
0.265450662 | 2.5.57 | PLA2G16 | 2.5.57 | phospholipase A2 group XVI | 2.039006238 | 2.5.57 ||
PLIN5 | 2.5.57 | perilipin 5 | 0.517786657 | 2.5.57 || PLXDC2 | 2.5.57 | plexin domain containing 2 |
0.412229253 | 2.5.57 || POLI | 2.5.57 | polymerase (DNA) iota | 0.924882514 | 2.5.57 || PPARG |
2.5.57 | peroxisome proliferator activated receptor gamma | 1.453567624 | 2.5.57 || PPP2R1B |
2.5.57 | protein phosphatase 2 regulatory subunit A, beta | 1.505337477 | 2.5.57 || RALGAPA2 |
2.5.57 | Ral GTPase activating protein catalytic alpha subunit 2 | 0.738386312 | 2.5.57 || RBP4 |
2.5.57 | retinol binding protein 4 | 3.619055585 | 2.5.57 || RBPMS | 2.5.57 | RNA binding protein
with multiple splicing | 0.886181558 | 2.5.57 || SELENBP1 | 2.5.57 | selenium binding protein 1 |
1.409902949 | 2.5.57 || SEMA3G | 2.5.57 | semaphorin 3G | 1.798415431 | 2.5.57 || SESTD1 |
2.5.57 | SEC14 and spectrin domain containing 1 | 0.453768368 | 2.5.57 || SGCE | 2.5.57 |
sarcoglycan epsilon | 1.422041389 | 2.5.57 || SH3D19 | 2.5.57 | SH3 domain containing 19 |
0.590620952 | 2.5.57 || SIK2 | 2.5.57 | salt inducible kinase 2 | 1.100510344 | 2.5.57 || SLC25A20 |
2.5.57 | solute carrier family 25 member 20 | 0.287242627 | 2.5.57 || SLC25A33 | 2.5.57 | solute
carrier family 25 member 33 | 0.756888187 | 2.5.57 || SLC9A1 | 2.5.57 | solute carrier family 9
member Al | 0.22427177 | 2.5.57 || SORL1 | 2.5.57 | sortilin-related receptor, L(DLR class) A
repeats containing | 0.929365898 | 2.5.57 || SPTBN1 | 2.5.57 | spectrin beta, non-erythrocytic 1 |
0.961247885 | 2.5.57 || TAPT1 | 2.5.57 | transmembrane anterior posterior transformation 1 |
0.346661131 | 2.5.57 || TBC1D2 | 2.5.57 | TBC1 domain family member 2 | 0.609191709 | 2.5.57 ||
TEAD1 | 2.5.57 | TEA domain transcription factor 1 | 0.797688257 | 2.5.57 || TMEM135 | 2.5.57 |
transmembrane protein 135 | 1.023373064 | 2.5.57 || TOB1 | 2.5.57 | transducer of ERBB2, 1 |
0.279597861 | 2.5.57 || TSKU | 2.5.57 | tsukushi, small leucine rich proteoglycan | 0.828512375 |
2.5.57 || USF3 | 2.5.57 | upstream transcription factor family member 3 | 0.342030412 | 2.5.57 ||
ACOT13 | 3.29 | acyl-CoA thioesterase 13 | 0.370379542 | 3.29.160 || ARID3A | 3.29 | AT-rich
interaction domain 3A | 0.38604131 | 3.29.160 || DANCR | 3.29 | differentiation antagonizing non-
protein coding RNA | 0.492106201 | - || DEXI | 3.29 | Dexi homolog (mouse) | 0.358144674 | - ||
DNAJC7 | 3.29 | DnaJ heat shock protein family (Hsp40) member C7 | 0.236518842 | - || DOCK5 |
3.29 | dedicator of cytokinesis 5 | 0.818765291 | - || EP300 | 3.29 | ElA binding protein p300 |
0.380938161 | 3.29.160 || FIS1 | 3.29 | fission, mitochondrial 1 | 0.288441357 | 3.29.160 || GCNT7 |
3.29 | glucosaminyl (N-acetyl) transferase family member 7 | 0.238017495 | 3.29.160 || GPX7 | 3.29
| glutathione peroxidase 7 | 0.750372703 | 3.29.160 || LARP4B | 3.29 | La ribonucleoprotein domain
family member 4B | 0.351785718 | 3.29.160 || MCEE | 3.29 | methylmalonyl-CoA epimerase |
0.376691829 | 3.29.160 || MILR1 | 3.29 | mast cell immunoglobulin-like receptor 1 | 0.533631543 | -
|| MRPL48 | 3.29 | mitochondrial ribosomal protein L48 | 0.337494272 | 3.29.160 || N4BP2L2 | 3.29
| NEDD4 binding protein 2-like 2 | 0.300221207 | 3.29.160 || NAA38 | 3.29 | N(alpha)-
acetyltransferase 38, NatC auxiliary subunit | 0.259988906 | 3.29.160 || NAXE | 3.29 | NAD(P)HX
epimerase | 0.240081124 | - || NDUFA13 | 3.29 | NADH: ubiquinone oxidoreductase subunit A13 |
0.226874413 | 3.29.160 || NME1 | 3.29 | NME/NM23 nucleoside diphosphate kinase 1 |
0.466163737 | 3.29.160 || PEPD | 3.29 | peptidase D | 0.235898545 | - || PINK1 | 3.29 | PTEN
induced putative kinase 1 | 0.282007013 | 3.29.160 || PLGRKT | 3.29 | plasminogen receptor, C-
terminal lysine transmembrane protein | 0.405399761 | 3.29.160 || POP5 | 3.29 | POP5 homolog,
ribonuclease P/MRP subunit | 0.259027764 | 3.29.160 || PRPS1 | 3.29 | phosphoribosyl
pyrophosphate synthetase 1 | 0.233745789 | - || PTRHD1 | 3.29 | peptidyl-tRNA hydrolase domain
containing 1 | 0.279295428 | 3.29.160 || RNASEH2C | 3.29 | ribonuclease H2 subunit C |
0.341173901 | 3.29.160 || RPA3 | 3.29 | replication protein A3 | 0.267876142 | 3.29.160 || SMIM19 |
3.29 | small integral membrane protein 19 | 0.266262055 | 3.29.160 || SMYD3 | 3.29 | SET and
MYND domain containing 3 | 0.228881457 | - || TBRG1 | 3.29 | transforming growth factor beta
regulator 1 | 0.253776071 | 3.29.160 || TMEM261 | 3.29 | transmembrane protein 261 | 0.27175084 |
3.29.160 || TPSAB1 | 3.29 | tryptase alpha/beta 1 | 1.158017906 | - || TPSB2 | 3.29 | tryptase beta 2
(gene/pseudogene) | 1.616666898 | - || UQCRQ | 3.29 | ubiquinol-cytochrome c reductase complex
III subunit VII | 0.221191949 | 3.29.160 || WDR6 | 3.29 | WD repeat domain 6 | 0.253672531 | - ||
ZNF32 | 3.29 | zinc finger protein 32 | 0.241519498 | 3.29.160 || ACOT13 | 3.29.160 | acyl-CoA
thioesterase 13 | 0.370379542 | 3.29.160 || ARID3A | 3.29.160 | AT-rich interaction domain 3A |
0.38604131 | 3.29.160 || EP300 | 3.29.160 | ElA binding protein p300 | 0.380938161 | 3.29.160 ||
FIS1 | 3.29.160 || fission, mitochondrial 1 | 0.288441357 | 3.29.160 || GCNT7 | 3.29.160 |
glucosaminyl (N-acetyl) transferase family member 7 | 0.238017495 | 3.29.160 || GPX7 | 3.29.160 |
glutathione peroxidase 7 | 0.750372703 | 3.29.160 || LARP4B | 3.29.160 | La ribonucleoprotein
domain family member 4B | 0.351785718 | 3.29.160 || MCEE | 3.29.160 | methylmalonyl-CoA
epimerase | 0.376691829 | 3.29.160 || MRPL48 | 3.29.160 | mitochondrial ribosomal protein L48 |
0.337494272 | 3.29.160 || N4BP2L2 | 3.29.160 | NEDD4 binding protein 2-like 2 | 0.300221207 |
3.29.160 || NAA38 | 3.29.160 | N(alpha)-acetyltransferase 38, NatC auxiliary subunit | 0.259988906
| 3.29.160 || NDUFA13 | 3.29.160 | NADH:ubiquinone oxidoreductase subunit A13 | 0.226874413 |
3.29.160 || NME1 | 3.29.160 | NME/NM23 nucleoside diphosphate kinase 1 | 0.466163737 |
3.29.160 || PINK1 | 3.29.160 | PTEN induced putative kinase 1 | 0.282007013 | 3.29.160 || PLGRKT
| 3.29.160 | plasminogen receptor, C-terminal lysine transmembrane protein | 0.405399761 |
3.29.160 || POP5 | 3.29.160 | POP5 homolog, ribonuclease P/MRP subunit | 0.259027764 | 3.29.160
|| PTRHD1 | 3.29.160 | peptidyl-tRNA hydrolase domain containing 1 | 0.279295428 | 3.29.160 ||
RNASEH2C | 3.29.160 | ribonuclease H2 subunit C | 0.341173901 | 3.29.160 || RPA3 | 3.29.160 |
replication protein A3 | 0.267876142 | 3.29.160 || SMIM19 | 3.29.160 | small integral membrane
protein 19 | 0.266262055 | 3.29.160 || TBRG1 | 3.29.160 | transforming growth factor beta regulator
1 | 0.253776071 | 3.29.160 || TMEM261 | 3.29.160 | transmembrane protein 261 | 0.27175084 |
3.29.160 || UQCRQ | 3.29.160 | ubiquinol-cytochrome c reductase complex III subunit VII |
0.221191949 | 3.29.160 || ZNF32 | 3.29.160 | zinc finger protein 32 | 0.241519498 | 3.29.160 ||
AAAS | 3.30 | aladin WD repeat nucleoporin | 0.352373522 | 3.30.161 || AAK1 | 3.30 | AP2
associated kinase 1 | 0.358812961 | 3.30.163 || AASS | 3.30 | aminoadipate-semialdehyde synthase |
1.282105419 | 3.30.164 || ABCF2 | 3.30 | ATP binding cassette subfamily F member 2 | 0.22823075
| 3.30.163 || ACO2 | 3.30 | aconitase 2 | 0.295624058 | 3.30.161 || ADAM15 | 3.30 | ADAM
metallopeptidase domain 15 | 0.346230317 | 3.30.161 || ADAM17 | 3.30 | ADAM metallopeptidase
domain 17 | 0.31099106 | - || ADAM33 | 3.30 | ADAM metallopeptidase domain 33 | 0.254840952 |
3.30.161 || ADAT1 | 3.30 | adenosine deaminase, tRNA-specific 1 | 0.52589383 | 3.30.161 || AGA |
3.30 | aspartylglucosaminidase | 0.233971109 | 3.30.162 || AGBL5 | 3.30 | ATP/GTP binding
protein-like 5 | 0.350600851 | 3.30.161 || AGTPBP1 | 3.30 | ATP/GTP binding protein 1 |
0.408045446 | - || ALDHIB1 | 3.30 | aldehyde dehydrogenase 1 family member B1 | 0.307312348 |
3.30.161 || ALMS1 | 3.30 | ALMS1, centrosome and basal body associated protein | 0.317654559 |
3.30.161 || ALPK1 | 3.30 | alpha kinase 1 | 0.335151621 | 3.30.161 || ANKHD1 | 3.30 | ankyrin
repeat and KH domain containing 1 | 0.729834743 | - || ANKLE2 | 3.30 | ankyrin repeat and LEM
domain containing 2 | 0.456517817 | 3.30.161 || ANKRD12 | 3.30 | ankyrin repeat domain 12 |
0.318898415 | - || AP1AR | 3.30 | adaptor related protein complex 1 associated regulatory protein |
0.673226766 | 3.30.161 || APOPT1 | 3.30 | apoptogenic 1, mitochondrial | 0.268376405 | 3.30.161 ||
ARID2 | 3.30 | AT-rich interaction domain 2 | 0.330695084 | 3.30.161 || ARIH2 | 3.30 | ariadne
RBR E3 ubiquitin protein ligase 2 | 0.368630654 | 3.30.164 || ARMC9 | 3.30 | armadillo repeat
containing 9 | 0.396598752 | - || ASXL1 | 3.30 | additional sex combs like 1, transcriptional
regulator | 0.715793671 | 3.30.161 || ATAD3B | 3.30 | ATPase family, AAA domain containing 3B |
0.271465067 | 3.30.161 || ATF4 | 3.30 | activating transcription factor 4 | 0.216035027 | 3.30.161 ||
ATP8B1 | 3.30 | ATPase phospholipid transporting 8B1 | 0.43453393 | 3.30.161 || ATXN7L1 | 3.30
| ataxin 7 like 1 | 0.249254691 | - || AZI2 | 3.30 | 5-azacytidine induced 2 | 0.364985673 | 3.30.161 ||
B4GALT3 | 3.30 | UDP-Gal:betaGlcNAc beta 1,4- galactosyltransferase, polypeptide 3 |
0.262314081 | - || BCL2L1 | 3.30 | BCL2 like 1 | 0.47724347 | 3.30.161 || BLOC1S3 | 3.30 |
biogenesis of lysosomal organelles complex 1 subunit 3 | 0.239303652 | 3.30.161 || BLZF1 | 3.30 |
basic leucine zipper nuclear factor 1 | 0.358699956 || - || BMP7 | 3.30 | bone morphogenetic protein 7
| 0.245166177 | 3.30.161 || BMS1P5 | 3.30 | BMS1, ribosome biogenesis factor pseudogene 5 |
0.681368074 | 3.30.162 || BORCS6 | 3.30 | BLOC-1 related complex subunit 6 | 0.253484607 |
3.30.164 || BPTF | 3.30 | bromodomain PHD finger transcription factor | 0.305189338 | 3.30.161 ||
BSG | 3.30 | basigin (Ok blood group) | 0.316299297 | 3.30.163 || C12orf43 | 3.30 | chromosome 12
open reading frame 43 | 0.606791983 | 3.30.164 || C19orf60 | 3.30 | chromosome 19 open reading
frame 60 | 0.247303532 | 3.30.161 || C20orf194 | 3.30 | chromosome 20 open reading frame 194 |
0.496257029 | 3.30.164 || C2orf68 | 3.30 | chromosome 2 open reading frame 68 | 1.19619938 |
3.30.161 || C9orf64 | 3.30 | chromosome 9 open reading frame 64 | 0.343659592 | 3.30.161 ||
CAMKMT | 3.30 | calmodulin-lysine N-methyltransferase | 0.265474786 | - || CAPS2 | 3.30 |
calcyphosine 2 | 0.871079797 | - || CASP10 | 3.30 | caspase 10 | 0.592607805 | 3.30.161 || CCDC152
| 3.30 | coiled-coil domain containing 152 | 0.294292919 | 3.30.161 || CCM2 | 3.30 | CCM2
scaffolding protein | 0.317281828 | 3.30.163 || CCZ1B | 3.30 | CCZ1 homolog B, vacuolar protein
trafficking and biogenesis associated | 0.349017772 | 3.30.163 || CDK13 | 3.30 | cyclin-dependent
kinase 13 | 0.576340437 | 3.30.161 || CDK2AP2 | 3.30 | cyclin-dependent kinase 2 associated
protein 2 | 0.430001629 | 3.30.161 || CDK5RAP1 | 3.30 | CDK5 regulatory subunit associated
protein 1 | 0.360318468 | - || CENPBD1 | 3.30 | CENPB DNA-binding domain containing 1 |
0.374199987 | 3.30.161 || CEP152 | 3.30 | centrosomal protein 152kDa | 0.260393368 | 3.30.162 ||
CEP76 | 3.30 | centrosomal protein 76kDa | 0.267774115 | - || CEP89 | 3.30 | centrosomal protein
89kDa | 0.746193621 | 3.30.162 || CIB1 | 3.30 | calcium and integrin binding 1 | 0.330967419 | - ||
CLEC12A | 3.30 | C-type lectin domain family 12 member A | 4.275025327 | - || CNPY3 | 3.30 |
canopy FGF signaling regulator 3 | 0.274351945 | 3.30.161 || CNTLN | 3.30 | centlein | 1.530856311
| 3.30.161 || CRIPT | 3.30 | CXXC repeat containing interactor of PDZ3 domain | 0.421443808 |
3.30.163 || CRYBB2P1 | 3.30 | crystallin beta B2 pseudogene 1 | 0.261306653 | 3.30.161 || CUTC |
3.30 | cutC copper transporter | 0.486947462 | - || CXorf21 | 3.30 | chromosome X open reading
frame 21 | 0.331323197 | 3.30.162 || CYB5R1 | 3.30 | cytochrome b5 reductase 1 | 0.421170473 | - ||
CYC1 | 3.30 | cytochrome cl | 0.223575465 | 3.30.161 || DAPP1 | 3.30 | dual adaptor of
phosphotyrosine and 3-phosphoinositides | 0.35628854 | 3.30.161 || DBT | 3.30 | dihydrolipoamide
branched chain transacylase E2 | 0.310379857 | 3.30.161 || DDB2 | 3.30 | damage specific DNA
binding protein 2 | 0.241096702 | 3.30.161 || DEFB1 | 3.30 | defensin beta 1 | 4.132062917 | - ||
DHFRL1 | 3.30 | dihydrofolate reductase like 1 | 0.243966395 | 3.30.161 || DHPS | 3.30 |
deoxyhypusine synthase | 0.253298759 | 3.30.161 || DHRSX | 3.30 | dehydrogenase/reductase (SDR
family) X-linked | 0.477668615 | - || DIP2A | 3.30 | disco interacting protein 2 homolog A |
0.622451462 | 3.30.161 || DLGAP4 | 3.30 | discs large homolog associated protein 4 | 0.512951009 |
3.30.161 || DNAJC27 | 3.30 | DnaJ heat shock protein family (Hsp40) member C27 | 0.310844827 |
3.30.161 || E2F3 | 3.30 | E2F transcription factor 3 | 0.259237654 | - || EBLN3 | 3.30 | endogenous
Bornavirus-like nucleoprotein 3 | 0.259890806 | 3.30.163 || EIF3C | 3.30 | eukaryotic translation
initiation factor 3 subunit C | 1.718522589 | - || ELAVL3 | 3.30 | ELAV like neuron-specific RNA
binding protein 3 | 0.538596786 | 3.30.164 || EPHB4 | 3.30 | EPH receptor B4 | 0.526923635 |
3.30.161 || ETHE1 | 3.30 | ethylmalonic encephalopathy 1 | 0.447625879 | 3.30.162 || EXOSC2 |
3.30 | exosome component 2 | 0.285655288 | 3.30.161 || FAM155A | 3.30 | family with sequence
similarity 155 member A | 0.845409308 | - || FAM161B | 3.30 | family with sequence similarity 161
member B | 0.351430932 | 3.30.161 || FAM173B | 3.30 | family with sequence similarity 173
member B | 0.778665319 | - || FAM185A | 3.30 | family with sequence similarity 185 member A |
0.409938388 | 3.30.161 || FAM50A | 3.30 | family with sequence similarity 50 member A |
0.275008177 | 3.30.161 || FAM63A | 3.30 | family with sequence similarity 63 member A |
0.309692891 | 3.30.161 || FARSA | 3.30 | phenylalanyl-tRNA synthetase alpha subunit
0.243472131 | 3.30.161 || FBXL14 | 3.30 | F-box and leucine-rich repeat protein 14 | 0.24496978 |
3.30.164 || FBXW12 | 3.30 | F-box and WD repeat domain containing 12 | 0.215501759 | 3.30.161 ||
FCF1 | 3.30 | FCF1 rRNA-processing protein | 1.564275516 | 3.30.161 || FJX1 | 3.30 | four jointed
box 1 | 0.393358658 | - || FLJ35934 | 3.30 | FLJ35934 | 1.465558321 | 3.30.161 || FMO4 | 3.30 |
flavin containing monooxygenase 4 | 0.279914387 | - || FOLR2 | 3.30 | folate receptor 2 (fetal) |
0.224800243 | - || FOXC2 | 3.30 | forkhead box C2 | 0.494783451 | 3.30.161 || FTX | 3.30 | FTX
transcript, XIST regulator (non-protein coding) | 0.367138006 | 3.30.161 || FUCA1 | 3.30 |
fucosidase, alpha-L- 1, tissue | 0.351798571 | - || GAS2L3 | 3.30 | growth arrest specific 2 like 3 |
0.457955472 | - || GDAP2 | 3.30 | ganglioside induced differentiation associated protein 2 |
0.245697891 | 3.30.161 || GLUD2 | 3.30 | glutamate dehydrogenase 2 | 0.515300288 | 3.30.161 ||
GPATCH2L | 3.30 | G-patch domain containing 2 like | 1.960179904 | 3.30.163 || GPR180 | 3.30 | G
protein-coupled receptor 180 | 0.700540683 | 3.30.161 || GPX1 | 3.30 | glutathione peroxidase 1 |
0.217753186 | - || GRSF1 | 3.30 | G-rich RNA sequence binding factor 1 | 0.2286843 | 3.30.161 ||
GTF2H3 | 3.30 | general transcription factor IIH subunit 3 | 0.466118302 | 3.30.162 || GUCD1 | 3.30
| guanylyl cyclase domain containing 1 | 0.236375233 | 3.30.161 || H6PD | 3.30 | hexose-6-
phosphate dehydrogenase (glucose 1-dehydrogenase) | 0.420292436 | 3.30.163 || HACD4 | 3.30 | 3-
hydroxyacyl-CoA dehydratase 4 | 0.219712177 | 3.30.161 || HIC2 | 3.30 | hypermethylated in cancer
2 | 0.648115152 | - || HOMER3 | 3.30 | homer scaffolding protein 3 | 0.432097501 | 3.30.162 ||
HOXC9 | 3.30 | homeobox C9 | 0.439238775 | 3.30.164 || HSCB | 3.30 | HscB mitochondrial iron-
sulfur cluster co-chaperone | 0.401953843 | 3.30.161 || ISG20L2 | 3.30 | interferon stimulated
exonuclease gene 20kDa like 2 | 0.577257953 | 3.30.161 || KANSLIL | 3.30 | KAT8 regulatory NSL
complex subunit 1 like | 0.58843994 | 3.30.161 || KATNB1 | 3.30 | katanin p80 (WD repeat
containing) subunit B 1 | 0.282564914 | 3.30.161 || KIFC3 | 3.30 | kinesin family member C3 |
0.683956714 | - || KLF8 | 3.30 | Kruppel-like factor 8 | 0.94019415 | 3.30.161 || KXD1 | 3.30 | KxDL
motif containing 1 | 0.255973442 | 3.30.161 || LARS | 3.30 | leucyl-tRNA synthetase | 0.269634877 |
3.30.161 || LIME1 | 3.30 | Lck interacting transmembrane adaptor 1 | 0.219325616 | 3.30.161 ||
LINS1 | 3.30 | lines homolog 1 | 1.661817972 | - || LMAN2 | 3.30 | lectin, mannose binding 2 |
0.27351954 | 3.30.161 || LOC100506282 | 3.30 | uncharacterized LOC100506282 | 0.967848883 |
3.30.162 || LOC388692 | 3.30 | uncharacterized LOC388692 | 0.247042414 | 3.30.161 || LSM10 |
3.30 | LSM10, U7 small nuclear RNA associated | 0.372634322 | - || LSM4 | 3.30 | LSM4 homolog,
U6 small nuclear RNA and mRNA degradation associated | 0.295416034 | 3.30.161 || MAP3K2 |
3.30 | mitogen-activated protein kinase kinase kinase 2 | 0.239860097 | - || MAPRE3 | 3.30 |
microtubule associated protein RP/EB family member 3 | 0.357711959 | 3.30.164 || MBIP | 3.30|
MAP3K12 binding inhibitory protein 1 | 0.707044418 | 3.30.164 || MCM5 | 3.30 | minichromosome
maintenance complex component 5 | 0.311663026 | 3.30.161 || MED1 | 3.30 | mediator complex
subunit 1 | 0.437689844 | 3.30.161 || MED10 | 3.30 | mediator complex subunit 10 | 0.318993623 |
3.30.161 || MED25 | 3.30 | mediator complex subunit 25 | 0.283174309 | 3.30.161 || MFNG | 3.30 |
MFNG O-fucosylpeptide 3-beta-N-acetylglucosaminyltransferase | 0.281534973 | 3.30.161 ||
MFSD4B | 3.30 | major facilitator superfamily domain containing 4B | 0.426375024 | 3.30.162 ||
MIB1 | 3.30 | mindbomb E3 ubiquitin protein ligase 1 | 0.36953511 | 3.30.161 || MOB3B | 3.30
MOB kinase activator 3B | 0.411911627 | - || MRPL42 | 3.30 | mitochondrial ribosomal protein L42
| 0.352435304 | - || MRPS25 | 3.30 | mitochondrial ribosomal protein S25 | 0.346073909 | 3.30.161 ||
MRPS34 | 3.30 | mitochondrial ribosomal protein S34 | 0.305962574 | 3.30.161 || MTRFIL | 3.30 |
mitochondrial translational release factor 1 like | 0.512985038 | 3.30.164 || MUM1 | 3.30 |
melanoma associated antigen (mutated) 1 | 0.846361478 | 3.30.161 || MYBL2 | 3.30 | MYB proto-
oncogene like 2 | 0.321355477 | 3.30.161 || NADSYN1 | 3.30 | NAD synthetase 1 | 0.276552096 | -
|| NANOG | 3.30 | Nanog homeobox | 0.969002288 | 3.30.161 || NBEA | 3.30 | neurobeachin |
0.319732489 | 3.30.161 || NDUFAF7 | 3.30 | NADH: ubiquinone oxidoreductase complex assembly
factor 7 | 0.275815248 | 3.30.161 || NLGN4X | 3.30 | neuroligin 4, X-linked | 0.228910945 |
3.30.161 || NLN | 3.30 | neurolysin | 0.526426523 | 3.30.161 || NME6 | 3.30 | NME/NM23
nucleoside diphosphate kinase 6 | 0.620866214 | 3.30.161 || NMT1 | 3.30 | N-myristoyltransferase 1
| 0.344492238 | 3.30.164 || NOP10 | 3.30 | NOP10 ribonucleoprotein | 0.23502896 | 3.30.163 ||
NOP2 | 3.30 | NOP2 nucleolar protein | 0.274619566 | - || NOTCH2NL | 3.30 | notch 2 N-terminal
like | 0.316218069 | 3.30.161 || NOVA2 | 3.30 | neuro-oncological ventral antigen 2 | 0.332706539 |
3.30.161 || NPEPL1 | 3.30 | aminopeptidase-like 1 | 0.505830032 | 3.30.164 || NPRL2 | 3.30 | NPR2-
like, GATORI complex subunit | 0.259521118 | 3.30.162 || NPTN-IT1 | 3.30 | NPTN intronic
transcript 1 | 0.645011649 | 3.30.163 || NRIP1 | 3.30 | nuclear receptor interacting protein 1 |
0.540479072 | 3.30.164 || NUB1 | 3.30 | negative regulator of ubiquitin-like proteins 1 |
0.223485752 | - || NUBPL | 3.30 | nucleotide binding protein like | 0.35114588 | 3.30.161 || NUDT2
| 3.30 | nudix hydrolase 2 | 0.242382319 | - || NUMBL | 3.30 | numb homolog (Drosophila)-like |
0.452337826 | 3.30.164 || NUP58 | 3.30 | nucleoporin 58kDa | 0.707272837 | 3.30.161 || NUP93 |
3.30 | nucleoporin 93kDa | 0.260078771 | - || NUTM2B-AS1 | 3.30 | NUTM2B antisense RNA 1 |
0.618545548 | 3.30.161 || NXPE3 | 3.30 | neurexophilin and PC-esterase domain family member 3 |
0.655944585 | 3.30.162 || OGFR | 3.30 | opioid growth factor receptor | 0.223730018 | 3.30.161 ||
ORAI2 | 3.30 | ORAI calcium release-activated calcium modulator 2 | 0.611215362 | 3.30.161 ||
ORMDL2 | 3.30 | ORMDL sphingolipid biosynthesis regulator 2 | 0.383004896 | 3.30.161 ||
OTUD6B | 3.30 | OTU domain containing 6B | 0.408954059 | 3.30.161 || PABPN1 | 3.30 | poly(A)
binding protein, nuclear 1 | 0.433609034 | 3.30.161 || PAK1 | 3.30 | p21 protein (Cdc42/Rac)-
activated kinase 1 | 0.255512807 | 3.30.161 || PALM2 | 3.30 | paralemmin 2 | 0.624064675 |
3.30.164 || PAXIP1-AS1 | 3.30 | PAXIPI antisense RNA 1 (head to head) | 0.381161759 | 3.30.161
|| PCBD2 | 3.30 | pterin-4 alpha-carbinolamine dehydratase 2 | 0.53520521 | - || PCBP1-AS1 | 3.30 |
PCBP1 antisense RNA 1 | 0.451561689 | 3.30.161 || PDCD7 | 3.30 | programmed cell death 7 |
0.365597024 | 3.30.161 || PDCL | 3.30 | phosducin like | 0.248924835 | 3.30.161 || PDE4DIP | 3.30 |
phosphodiesterase 4D interacting protein | 1.238864848 | - || PECR | 3.30 | peroxisomal trans-2-
enoyl-CoA reductase | 0.677321678 | 3.30.161 || PGF | 3.30 | placental growth factor | 0.389492364
| 3.30.161 || PHF19 | 3.30 | PHD finger protein 19 | 0.223424043 | 3.30.161 || PHF20L1 | 3.30 | PHD
finger protein 20-like 1 | 0.420513453 | - || PIEZO2 | 3.30 | piezo type mechanosensitive ion channel
component 2 | 1.544273829 | - || POLD4 | 3.30 | polymerase (DNA) delta 4, accessory subunit |
0.3957221 | 3.30.161 || POLRIB | 3.30 | polymerase (RNA) I subunit B | 0.796356992 | 3.30.161 ||
POLRMT | 3.30 | polymerase (RNA) mitochondrial | 0.310785506 | 3.30.161 || PPA2 | 3.30 |
pyrophosphatase (inorganic) 2 | 0.687462604 | 3.30.161 || PPHLN1 | 3.30 | periphilin 1 |
0.254201029 | 3.30.163 || PPP2R5D | 3.30 | protein phosphatase 2 regulatory subunit B′, delta |
0.296598581 | 3.30.161 || PPP5C | 3.30 | protein phosphatase 5 catalytic subunit | 0.27648773 |
3.30.161 || PRR11 | 3.30 | proline rich 11 | 0.387011318 | 3.30.161 || PTHLH | 3.30 | parathyroid
hormone-like hormone | 0.237342811 | - || RAB2B | 3.30 | RAB2B, member RAS oncogene family |
0.217029099 | 3.30.161 || RABEP1 | 3.30 | rabaptin, RAB GTPase binding effector protein 1 |
0.247033339 | 3.30.163 || RABGAP1 | 3.30 | RAB GTPase activating protein 1 | 0.283328283 |
3.30.161 || RAD1 | 3.30 | RADI checkpoint DNA exonuclease | 0.550238703 | 3.30.161 || RANGRF
| 3.30 | RAN guanine nucleotide release factor | 0.36937128 | - || RAPH1 | 3.30 | Ras association
(RalGDS/AF-6) and pleckstrin homology domains 1 | 0.246647221 | 3.30.164 | RBBP5 | 3.30 |
retinoblastoma binding protein 5 | 0.383802185 | 3.30.161 || RBM26-AS1 | 3.30 | RBM26 antisense
RNA 1 | 0.33348482 | - || RDH5 | 3.30 | retinol dehydrogenase 5 | 1.348755651 | 3.30.161 || REL |
3.30 | v-rel avian reticuloendotheliosis viral oncogene homolog | 0.585549063 | - || RFX3 | 3.30 |
regulatory factor X3 | 0.398367876 | 3.30.161 || RIT1 | 3.30 | Ras-like without CAAX 1 |
0.708957957 | 3.30.162 || RNGTT | 3.30 | RNA guanylyltransferase and 5′-phosphatase |
0.345966138 | 3.30.161 || RP2 | 3.30 | retinitis pigmentosa 2 (X-linked recessive) | 0.328088093 | - ||
RPRD2 | 3.30 | regulation of nuclear pre-mRNA domain containing 2 | 0.355487833 | 3.30.164 ||
RPS19BP1 | 3.30 | ribosomal protein S19 binding protein 1 | 0.232584469 | - || RRAGD | 3.30 | Ras
related GTP binding D | 0.370224662 | 3.30.161 || RSPRY1 | 3.30 | ring finger and SPRY domain
containing 1 | 0.303005833 | - || SAMD4B | 3.30 | sterile alpha motif domain containing 4B |
0.530246528 | 3.30.161 || SCAF4 | 3.30 | SR-related CTD-associated factor 4 | 1.133357927 |
3.30.161 || SCO1 | 3.30 | SCO1 cytochrome c oxidase assembly protein | 0.408620694 | 3.30.163 ||
SDHAF1 | 3.30 | succinate dehydrogenase complex assembly factor 1 | 0.341757346 | - ||
SEC14LIP1 | 3.30 | SEC14 like 1 pseudogene 1 | 0.755378479 | 3.30.161 || SEC22A | 3.30 | SEC22
homolog A, vesicle trafficking protein | 0.390611834 | 3.30.164 || SELK | 3.30 | selenoprotein K |
0.26075525 | - || SERPING1 | 3.30 | serpin peptidase inhibitor, clade G (C1 inhibitor), member 1 |
0.333217799 | 3.30.161 || SEZ6L2 | 3.30 | seizure related 6 homolog (mouse)-like 2 | 0.372348623 |
3.30.164 || SGK494 | 3.30 | uncharacterized serine/threonine-protein kinase SgK494 | 0.664146991 |
3.30.161 || SHISA4 | 3.30 | shisa family member 4 | 1.07176908 | - || SLC26A6 | 3.30 | solute carrier
family 26 member 6 | 0.467606888 | 3.30.161 || SLC35B4 | 3.30 | solute carrier family 35 member
B4 | 0.224178696 | 3.30.161 || SLC35D1 | 3.30 | solute carrier family 35 member D1 | 0.355639249
| 3.30.162 | SLC35E1 | 3.30 | solute carrier family 35 member El | 0.297085088 | 3.30.161 ||
SMIM12 | 3.30 | small integral membrane protein 12 | 0.232600441 | - || SMIM8 | 3.30 | small
integral membrane protein 8 | 0.286061502 | 3.30.161 || SMO | 3.30 | smoothened, frizzled class
receptor | 0.627909031 | 3.30.163 || SNRNP200 | 3.30 | small nuclear ribonucleoprotein U5 subunit
200 | 0.738522656 | 3.30.161 || SNRNP25 | 3.30 | small nuclear ribonucleoprotein U11/U12 subunit
25 | 0.558607689 | 3.30.161 || SNX1 | 3.30 | sorting nexin 1 | 0.334783242 | - || SORBS1 | 3.30 |
sorbin and SH3 domain containing 1 | 0.297206207 | 3.30.161 || SPRY4-IT1 | 3.30 | SPRY4 intronic
transcript 1 | 0.272385534 | 3.30.162 || SREBF2 | 3.30 | sterol regulatory element binding
transcription factor 2 | 0.231625924 | 3.30.161 || SRFBP1 | 3.30 | serum response factor binding
protein 1 | 0.98837139 | 3.30.161 || SRGAP1 | 3.30 | SLIT-ROBO Rho GTPase activating protein 1 |
0.690167007 | 3.30.161 || SSBP3-AS1 | 3.30 | SSBP3 antisense RNA 1 | 0.373058087 | 3.30.161 |
SSNA1 | 3.30 | Sjogren syndrome nuclear autoantigen 1 | 0.228474379 | 3.30.163 || STARD10 |
3.30 | StAR related lipid transfer domain containing 10 | 0.3466871 | 3.30.161 || STIP1 | 3.30 | stress
induced phosphoprotein 1 | 0.301017828 | - || STRN | 3.30 | striatin | 0.741047152 | 3.30.161 ||
STX16 | 3.30 | syntaxin 16 | 0.412854668 | 3.30.161 || STXBP5 | 3.30 | syntaxin binding protein 5 |
1.089528566 | 3.30.164 || SUSD1 | 3.30 | sushi domain containing 1 | 0.485327403 | 3.30.161 ||
SYMPK | 3.30 | symplekin | 0.323207194 | - || TBC1D32 | 3.30 | TBC1 domain family member 32 |
0.324652475 | 3.30.164 || TBC1D5 | 3.30 | TBC1 domain family member 5 | 0.404257669 | - ||
TCAF1 | 3.30 | TRPM8 channel-associated factor 1 | 0.347099512 | 3.30.161 || TCP11L2 | 3.30 | t-
complex 11, testis-specific-like 2 | 0.363127472 | - || TCTN2 | 3.30 | tectonic family member 2 |
0.439404652 | 3.30.161 || TFAM | 3.30 | transcription factor A, mitochondrial | 0.665120986 | - ||
THUMPD3-AS1 | 3.30 | THUMPD3 antisense RNA 1 | 0.244344561 | 3.30.161 || TM9SF1 | 3.30
transmembrane 9 superfamily member 1 | 0.330628147 | 3.30.161 || TMED1 | 3.30 | transmembrane
p24 trafficking protein 1 | 0.320024357 | - || TMEM160 | 3.30 | transmembrane protein 160 |
0.38000783 | - || TMEM209 | 3.30 | transmembrane protein 209 | 0.46025373 | 3.30.163 ||
TMEM260 | 3.30 | transmembrane protein 260 | 0.320205953 | - || TMEM67 | 3.30 | transmembrane
protein 67 | 0.677453744 | 3.30.164 || TMPRSS6 | 3.30 | transmembrane protease, serine 6 |
0.399668197 | 3.30.161 || TNFRSF10D | 3.30 | tumor necrosis factor receptor superfamily member
10d | 0.339928148 | - || TOLLIP | 3.30 | toll interacting protein | 0.358870057 | 3.30.161 || TOPORS
| 3.30 | topoisomerase I binding, arginine/serine-rich, E3 ubiquitin protein ligase | 0.367545283 |
3.30.161 | TORIB | 3.30 | torsin family 1 member B | 0.293724707 | 3.30.161 || TRA2A | 3.30 |
transformer 2 alpha homolog (Drosophila) | 0.704028796 | 3.30.163 || TRIM38 | 3.30 | tripartite
motif containing 38 | 0.229195649 | 3.30.161 || TRIM66 | 3.30 | tripartite motif containing 66 |
0.875678341 | - || TRMT10B | 3.30 | tRNA methyltransferase 10B | 0.260649423 | 3.30.162 ||
TSC22D2 | 3.30 | TSC22 domain family member 2 | 0.422337416 | - || TSEN2 | 3.30 | tRNA
splicing endonuclease subunit 2 | 0.40337825 | 3.30.161 || TSR1 | 3.30 | TSR1, 20S rRNA
accumulation, homolog (S. cerevisiae) | 0.231857811 | 3.30.161 || TTC32 | 3.30 | tetratricopeptide
repeat domain 32 | 0.363074357 | 3.30.161 || TTC5 | 3.30 | tetratricopeptide repeat domain 5 |
0.310589861 | 3.30.162 || TXNDC17 | 3.30 | thioredoxin domain containing 17 | 0.220925814 |
3.30.163 || UACA | 3.30 | uveal autoantigen with coiled-coil domains and ankyrin repeats |
0.246988249 | 3.30.161 || UBAC1 | 3.30 | UBA domain containing 1 | 0.214671317 | 3.30.161 ||
UBE2G1 | 3.30 | ubiquitin conjugating enzyme E2G 1 | 0.291900447 | - || UBE2S | 3.30 | ubiquitin
conjugating enzyme E2S | 0.268642983 | 3.30.161 || UBE3D | 3.30 | ubiquitin protein ligase E3D |
0.606740871 | 3.30.162 || UBN2 | 3.30 | ubinuclein 2 | 0.436643149 | 3.30.164 || UBXN2A | 3.30 |
UBX domain protein 2A | 0.332342825 | 3.30.161 || UCKL1 | 3.30 | uridine-cytidine kinase 1-like 1
| 0.301351666 | 3.30.162 || USP34 | 3.30 | ubiquitin specific peptidase 34 | 0.440042036 | 3.30.161 ||
VAMP2 | 3.30 | vesicle associated membrane protein 2 | 0.389730645 | 3.30.164 || VGLL4 | 3.30 |
vestigial like family member 4 | 0.273340825 | 3.30.164 || VHL | 3.30 | von Hippel-Lindau tumor
suppressor | 0.599904226 | 3.30.162 || VTIIA | 3.30 | vesicle transport through interaction with t-
SNAREs 1A | 0.477960914 | 3.30.161 || WASF1 | 3.30 | WAS protein family member 1 |
0.455656022 | 3.30.164 || WDPCP | 3.30 | WD repeat containing planar cell polarity effector |
0.521484164 | 3.30.161 || WDR92 | 3.30 | WD repeat domain 92 | 0.604784359 | 3.30.161 || WSB1 |
3.30 | WD repeat and SOCS box containing 1 | 0.347045553 | - || WTAP | 3.30 | Wilms tumor 1
associated protein | 0.324388067 | 3.30.161 || WWP1 | 3.30 | WW domain containing E3 ubiquitin
protein ligase 1 | 0.267222347 | 3.30.161 || ZC3HAVIL | 3.30 | zinc finger CCCH-type containing,
antiviral 1 like | 0.955106743 | - || ZDHHC16 | 3.30 | zinc finger DHHC-type containing 16 |
0.257332465 | 3.30.162 || ZGPAT | 3.30 | zinc finger CCCH-type and G-patch domain containing |
0.35496962 | 3.30.161 || ZNF107 | 3.30 | zinc finger protein 107 | 0.48019711 | - || ZNF136 | 3.30 |
zinc finger protein 136 | 0.741766055 | 3.30.161 || ZNF160 | 3.30 | zinc finger protein 160 |
0.472219291 | 3.30.161 || ZNF185 | 3.30 | zinc finger protein 185 (LIM domain) | 0.243878375 |
3.30.161 || ZNF230 | 3.30 | zinc finger protein 230 | 0.531498798 | 3.30.162 || ZNF275 | 3.30 | zinc
finger protein 275 | 0.381879871 | 3.30.161 || ZNF281 | 3.30 | zinc finger protein 281 | 0.303494188
| - || ZNF3 | 3.30 | zinc finger protein 3 | 0.907549536 | - || ZNF333 | 3.30 | zinc finger protein 333 |
1.010036815 | 3.30.161 || ZNF37A | 3.30 | zinc finger protein 37A | 0.341349617 | 3.30.161 ||
ZNF37BP | 3.30 | zinc finger protein 37B, pseudogene | 1.791939962 | 3.30.161 || ZNF431 | 3.30 |
zinc finger protein 431 | 2.274358104 | 3.30.161 || ZNF451 | 3.30 | zinc finger protein 451 |
0.287704099 | 3.30.161 || ZNF486 | 3.30 | zinc finger protein 486 | 0.235414336 | - || ZNF514 | 3.30
| zinc finger protein 514 | 0.975589962 | 3.30.161 | ZNF518A | 3.30 | zinc finger protein 518A |
0.505753968 | 3.30.161 || ZNF528 | 3.30 | zinc finger protein 528 | 0.364951731 | 3.30.161 ||
ZNF550 | 3.30 | zinc finger protein 550 | 0.275532844 | - || ZNF551 | 3.30 | zinc finger protein 551 |
1.023046204 | 3.30.161 || ZNF562 | 3.30 | zinc finger protein 562 | 0.389253803 | - || ZNF585A |
3.30 | zinc finger protein 585A | 0.437874075 | 3.30.161 || ZNF587 | 3.30 | zinc finger protein 587 |
0.216364592 | 3.30.161 || ZNF587B | 3.30 | zinc finger protein 587B | 0.259307402 | 3.30.161 ||
ZNF669 | 3.30 | zinc finger protein 669 | 0.390451114 | 3.30.163 || ZNF706 | 3.30 | zinc finger
protein 706 | 0.29526667 | 3.30.161 || ZNF721 | 3.30 | zinc finger protein 721 | 0.299909179 |
3.30.161 || ZNF75A | 3.30 | zinc finger protein 75a | 0.228062974 | 3.30.162 || ZNF785 | 3.30 | zinc
finger protein 785 | 2.268123854 | 3.30.164 || ZNF787 | 3.30 | zinc finger protein 787 | 0.292078686
| 3.30.161 || ZNF850 | 3.30 | zinc finger protein 850 | 0.279345017 | 3.30.162 || ZNRF1 | 3.30 | zinc
and ring finger 1, E3 ubiquitin protein ligase | 0.269880812 | - || AAAS | 3.30.161 | aladin WD
repeat nucleoporin | 0.352373522 | 3.30.161 || ACO2 | 3.30.161 | aconitase 2 | 0.295624058 |
3.30.161 || ADAM15 | 3.30.161 | ADAM metallopeptidase domain 15 | 0.346230317 | 3.30.161 ||
ADAM33 | 3.30.161 | ADAM metallopeptidase domain 33 | 0.254840952 | 3.30.161 || ADAT1 |
3.30.161 | adenosine deaminase, tRNA-specific 1 | 0.52589383 | 3.30.161 || AGBL5 | 3.30.161 |
ATP/GTP binding protein-like 5 | 0.350600851 | 3.30.161 || ALDHIB1 | 3.30.161 | aldehyde
dehydrogenase 1 family member B1 | 0.307312348 | 3.30.161 || ALMS1 | 3.30.161 | ALMS1,
centrosome and basal body associated protein | 0.317654559 | 3.30.161 || ALPK1 | 3.30.161 | alpha
kinase 1 | 0.335151621 | 3.30.161 || ANKLE2 | 3.30.161 | ankyrin repeat and LEM domain
containing 2 | 0.456517817 | 3.30.161 || APIAR | 3.30.161 | adaptor related protein complex 1
associated regulatory protein | 0.673226766 | 3.30.161 || APOPT1 | 3.30.161 | apoptogenic 1,
mitochondrial | 0.268376405 | 3.30.161 || ARID2 | 3.30.161 | AT-rich interaction domain 2 |
0.330695084 | 3.30.161 || ASXL1 | 3.30.161 | additional sex combs like 1, transcriptional regulator |
0.715793671 | 3.30.161 || ATAD3B | 3.30.161 | ATPase family, AAA domain containing 3B |
0.271465067 | 3.30.161 || ATF4 | 3.30.161 | activating transcription factor 4 | 0.216035027 |
3.30.161 || ATP8B1 | 3.30.161 | ATPase phospholipid transporting 8B1 | 0.43453393 | 3.30.161 ||
AZI2 | 3.30.161 | 5-azacytidine induced 2 | 0.364985673 | 3.30.161 || BCL2L1 | 3.30.161 | BCL2
like 1 | 0.47724347 | 3.30.161 || BLOC1S3 | 3.30.161 | biogenesis of lysosomal organelles complex
1 subunit 3 | 0.239303652 | 3.30.161 || BMP7 | 3.30.161 | bone morphogenetic protein 7 |
0.245166177 | 3.30.161 || BPTF | 3.30.161 | bromodomain PHD finger transcription factor |
0.305189338 | 3.30.161 || C19orf60 | 3.30.161 | chromosome 19 open reading frame 60 |
0.247303532 | 3.30.161 || C2orf68 | 3.30.161 | chromosome 2 open reading frame 68 | 1.19619938 |
3.30.161 || C9orf64 | 3.30.161 | chromosome 9 open reading frame 64 | 0.343659592 | 3.30.161 ||
CASP10 | 3.30.161 | caspase 10 | 0.592607805 | 3.30.161 || CCDC152 | 3.30.161 | coiled-coil
domain containing 152 | 0.294292919 | 3.30.161 || CDK13 | 3.30.161 | cyclin-dependent kinase 13 |
0.576340437 | 3.30.161 || CDK2AP2 | 3.30.161 | cyclin-dependent kinase 2 associated protein 2 |
0.430001629 | 3.30.161 || CENPBD1 | 3.30.161 | CENPB DNA-binding domain containing 1 |
0.374199987 | 3.30.161 || CNPY3 | 3.30.161 | canopy FGF signaling regulator 3 | 0.274351945 |
3.30.161 || CNTLN | 3.30.161 | centlein | 1.530856311 | 3.30.161 || CRYBB2P1 | 3.30.161 |
crystallin beta B2 pseudogene 1 | 0.261306653 | 3.30.161 || CYC1 | 3.30.161 | cytochrome cl |
0.223575465 | 3.30.161 || DAPP1 | 3.30.161 | dual adaptor of phosphotyrosine and 3-
phosphoinositides | 0.35628854 | 3.30.161 || DBT | 3.30.161 | dihydrolipoamide branched chain
transacylase E2 | 0.310379857 | 3.30.161 || DDB2 | 3.30.161 | damage specific DNA binding protein
2 | 0.241096702 | 3.30.161 || DHFRL1 | 3.30.161 | dihydrofolate reductase like 1 | 0.243966395 |
3.30.161 || DHPS | 3.30.161 | deoxyhypusine synthase | 0.253298759 | 3.30.161 || DIP2A | 3.30.161 |
disco interacting protein 2 homolog A | 0.622451462 | 3.30.161 || DLGAP4 | 3.30.161 | discs large
homolog associated protein 4 | 0.512951009 | 3.30.161 || DNAJC27 | 3.30.161 | DnaJ heat shock
protein family (Hsp40) member C27 | 0.310844827 | 3.30.161 || EPHB4 | 3.30.161 | EPH receptor
B4 | 0.526923635 | 3.30.161 || EXOSC2 | 3.30.161 | exosome component 2 | 0.285655288 | 3.30.161
|| FAM161B | 3.30.161 | family with sequence similarity 161 member B | 0.351430932 | 3.30.161 ||
FAM185A | 3.30.161 | family with sequence similarity 185 member A | 0.409938388 | 3.30.161 ||
FAM50A | 3.30.161 | family with sequence similarity 50 member A | 0.275008177 | 3.30.161 ||
FAM63A | 3.30.161 | family with sequence similarity 63 member A | 0.309692891 | 3.30.161 ||
FARSA | 3.30.161 | phenylalanyl-tRNA synthetase alpha subunit | 0.243472131 | 3.30.161 ||
FBXW12 | 3.30.161 | F-box and WD repeat domain containing 12 | 0.215501759 | 3.30.161 || FCF1
| 3.30.161 | FCF1 rRNA-processing protein | 1.564275516 | 3.30.161 || FLJ35934 | 3.30.161 |
FLJ35934 | 1.465558321 | 3.30.161 || FOXC2 | 3.30.161 | forkhead box C2 | 0.494783451 | 3.30.161
|| FTX | 3.30.161 | FTX transcript, XIST regulator (non-protein coding) | 0.367138006 | 3.30.161 ||
GDAP2 | 3.30.161 | ganglioside induced differentiation associated protein 2 | 0.245697891 |
3.30.161 || GLUD2 | 3.30.161 | glutamate dehydrogenase 2 | 0.515300288 | 3.30.161 || GPR180 |
3.30.161 | G protein-coupled receptor 180 | 0.700540683 | 3.30.161 || GRSF1 | 3.30.161 | G-rich
RNA sequence binding factor 1 | 0.2286843 | 3.30.161 || GUCD1 | 3.30.161 | guanylyl cyclase
domain containing 1 | 0.236375233 | 3.30.161 || HACD4 | 3.30.161 | 3-hydroxyacyl-CoA
dehydratase 4 | 0.219712177 | 3.30.161 || HSCB | 3.30.161 | HscB mitochondrial iron-sulfur cluster
co-chaperone | 0.401953843 | 3.30.161 || ISG20L2 | 3.30.161 | interferon stimulated exonuclease
gene 20kDa like 2 | 0.577257953 | 3.30.161 || KANSLIL | 3.30.161 | KAT8 regulatory NSL
complex subunit 1 like | 0.58843994 | 3.30.161 || KATNB1 | 3.30.161 | katanin p80 (WD repeat
containing) subunit B 1 | 0.282564914 | 3.30.161 || KLF8 | 3.30.161 | Kruppel-like factor 8 |
0.94019415 | 3.30.161 || KXD1 | 3.30.161 | KxDL motif containing 1 | 0.255973442 | 3.30.161 ||
LARS | 3.30.161 | leucyl-tRNA synthetase | 0.269634877 | 3.30.161 || LIME1 | 3.30.161 | Lck
interacting transmembrane adaptor 1 | 0.219325616 | 3.30.161 || LMAN2 | 3.30.161 | lectin,
mannose binding 2 | 0.27351954 | 3.30.161 || LOC388692 | 3.30.161 | uncharacterized LOC388692 |
0.247042414 | 3.30.161 | LSM4 | 3.30.161 | LSM4 homolog, U6 small nuclear RNA and mRNA
degradation associated | 0.295416034 | 3.30.161 || MCM5 | 3.30.161 | minichromosome
maintenance complex component 5 | 0.311663026 | 3.30.161 || MED1 | 3.30.161 | mediator complex
subunit 1 | 0.437689844 | 3.30.161 || MED10 | 3.30.161 | mediator complex subunit 10 |
0.318993623 | 3.30.161 || MED25 | 3.30.161 | mediator complex subunit 25 | 0.283174309 |
3.30.161 || MFNG | 3.30.161 | MFNG O-fucosylpeptide 3-beta-N-acetylglucosaminyltransferase |
0.281534973 | 3.30.161 || MIB1 | 3.30.161 | mindbomb E3 ubiquitin protein ligase 1 | 0.36953511 |
3.30.161 || MRPS25 | 3.30.161 | mitochondrial ribosomal protein S25 | 0.346073909 | 3.30.161 ||
MRPS34 | 3.30.161 | mitochondrial ribosomal protein S34 | 0.305962574 | 3.30.161 || MUM1 |
3.30.161 | melanoma associated antigen (mutated) 1 | 0.846361478 | 3.30.161 || MYBL2 | 3.30.161 |
MYB proto-oncogene like 2 | 0.321355477 | 3.30.161 || NANOG | 3.30.161 | Nanog homeobox |
0.969002288 | 3.30.161 || NBEA | 3.30.161 | neurobeachin | 0.319732489 | 3.30.161 || NDUFAF7 |
3.30.161 | NADH:ubiquinone oxidoreductase complex assembly factor 7 | 0.275815248 | 3.30.161 ||
NLGN4X | 3.30.161 | neuroligin 4, X-linked | 0.228910945 | 3.30.161 || NLN | 3.30.161 | neurolysin
| 0.526426523 | 3.30.161 || NME6 | 3.30.161 | NME/NM23 nucleoside diphosphate kinase 6 |
0.620866214 | 3.30.161 || NOTCH2NL | 3.30.161 | notch 2 N-terminal like | 0.316218069 | 3.30.161
|| NOVA2 | 3.30.161 | neuro-oncological ventral antigen 2 | 0.332706539 | 3.30.161 || NUBPL |
3.30.161 | nucleotide binding protein like | 0.35114588 | 3.30.161 || NUP58 | 3.30.161 | nucleoporin
58kDa | 0.707272837 | 3.30.161 || NUTM2B-AS1 | 3.30.161 | NUTM2B antisense RNA 1 |
0.618545548 | 3.30.161 || OGFR | 3.30.161 | opioid growth factor receptor | 0.223730018 | 3.30.161
|| ORAI2 | 3.30.161 | ORAI calcium release-activated calcium modulator 2 | 0.611215362 | 3.30.161
|| ORMDL2 | 3.30.161 | ORMDL sphingolipid biosynthesis regulator 2 | 0.383004896 | 3.30.161 ||
OTUD6B | 3.30.161 | OTU domain containing 6B | 0.408954059 | 3.30.161 || PABPN1 | 3.30.161 |
poly(A) binding protein, nuclear 1 | 0.433609034 | 3.30.161 || PAK1 | 3.30.161 | p21 protein
(Cdc42/Rac)-activated kinase 1 | 0.255512807 | 3.30.161 || PAXIP1-AS1 | 3.30.161 | PAXIP1
antisense RNA 1 (head to head) | 0.381161759 | 3.30.161| | PCBP1-AS1 | 3.30.161 | PCBP1
antisense RNA 1 | 0.451561689 | 3.30.161 || PDCD7 | 3.30.161 | programmed cell death 7 |
0.365597024 | 3.30.161 | PDCL | 3.30.161 | phosducin like | 0.248924835 | 3.30.161 || PECR |
3.30.161 | peroxisomal trans-2-enoyl-CoA reductase | 0.677321678 | 3.30.161 || PGF | 3.30.161 |
placental growth factor | 0.389492364 | 3.30.161 || PHF19 | 3.30.161 | PHD finger protein 19 |
0.223424043 | 3.30.161 || POLD4 | 3.30.161 | polymerase (DNA) delta 4, accessory subunit |
0.3957221 | 3.30.161 || POLRIB | 3.30.161 | polymerase (RNA) I subunit B | 0.796356992 |
3.30.161 || POLRMT | 3.30.161 | polymerase (RNA) mitochondrial | 0.310785506 | 3.30.161 ||
PPA2 | 3.30.161 | pyrophosphatase (inorganic) 2 | 0.687462604 | 3.30.161 || PPP2R5D | 3.30.161 |
protein phosphatase 2 regulatory subunit B′, delta | 0.296598581 | 3.30.161 || PPP5C | 3.30.161 |
protein phosphatase 5 catalytic subunit | 0.27648773 | 3.30.161 || PRR11 | 3.30.161 | proline rich 11
| 0.387011318 | 3.30.161 || RAB2B | 3.30.161 | RAB2B, member RAS oncogene family |
0.217029099 | 3.30.161 || RABGAP1 | 3.30.161 | RAB GTPase activating protein 1 | 0.283328283 |
3.30.161 || RAD1 | 3.30.161 | RADI checkpoint DNA exonuclease | 0.550238703 | 3.30.161 ||
RBBP5 | 3.30.161 | retinoblastoma binding protein 5 | 0.383802185 | 3.30.161 || RDH5 | 3.30.161
retinol dehydrogenase 5 | 1.348755651 | 3.30.161 || RFX3 | 3.30.161 | regulatory factor X3 |
0.398367876 | 3.30.161 || RNGTT | 3.30.161 | RNA guanylyltransferase and 5′-phosphatase |
0.345966138 | 3.30.161 || RRAGD | 3.30.161 | Ras related GTP binding D | 0.370224662 | 3.30.161
|| SAMD4B | 3.30.161 | sterile alpha motif domain containing 4B | 0.530246528 | 3.30.161 || SCAF4
| 3.30.161 | SR-related CTD-associated factor 4 | 1.133357927 | 3.30.161 || SEC14LIP1 | 3.30.161 |
SEC14 like 1 pseudogene 1 | 0.755378479 | 3.30.161 || SERPING1 | 3.30.161 | serpin peptidase
inhibitor, clade G (C1 inhibitor), member 1 | 0.333217799 | 3.30.161 || SGK494 | 3.30.161 |
uncharacterized serine/threonine-protein kinase SgK494 | 0.664146991 | 3.30.161 || SLC26A6
3.30.161 | solute carrier family 26 member 6 | 0.467606888 | 3.30.161 || SLC35B4 | 3.30.161 |
solute carrier family 35 member B4 | 0.224178696 | 3.30.161 || SLC35E1 | 3.30.161 | solute carrier
family 35 member El | 0.297085088 | 3.30.161 || SMIM8 | 3.30.161 | small integral membrane
protein 8 | 0.286061502 | 3.30.161||| SNRNP200 | 3.30.161 | small nuclear ribonucleoprotein U5
subunit 200 | 0.738522656 | 3.30.161 || SNRNP25 | 3.30.161 | small nuclear ribonucleoprotein
U11/U12 subunit 25 | 0.558607689 | 3.30.161 || SORBS1 | 3.30.161 | sorbin and SH3 domain
containing 1 | 0.297206207 | 3.30.161 || SREBF2 | 3.30.161 | sterol regulatory element binding
transcription factor 2 | 0.231625924 | 3.30.161 || SRFBP1 | 3.30.161 | serum response factor binding
protein 1 | 0.98837139 | 3.30.161 || SRGAP1 | 3.30.161 | SLIT-ROBO Rho GTPase activating
protein 1 | 0.690167007 | 3.30.161 || SSBP3-AS1 | 3.30.161 | SSBP3 antisense RNA 1 |
0.373058087 | 3.30.161 || STARD10 | 3.30.161 | StAR related lipid transfer domain containing 10 |
0.3466871 | 3.30.161 || STRN | 3.30.161 | striatin | 0.741047152 | 3.30.161 || STX16 | 3.30.161 |
syntaxin 16 | 0.412854668 | 3.30.161 || SUSD1 | 3.30.161 | sushi domain containing 1 | 0.485327403
| 3.30.161 || TCAF1 | 3.30.161 | TRPM8 channel-associated factor 1 | 0.347099512 | 3.30.161 ||
TCTN2 | 3.30.161 | tectonic family member 2 | 0.439404652 | 3.30.161 || THUMPD3-AS1 |
3.30.161 | THUMPD3 antisense RNA 1 | 0.244344561 | 3.30.161 || TM9SF1 | 3.30.161 |
transmembrane 9 superfamily member 1 | 0.330628147 | 3.30.161 || TMPRSS6 | 3.30.161 |
transmembrane protease, serine 6 | 0.399668197 | 3.30.161 || TOLLIP | 3.30.161 | toll interacting
protein | 0.358870057 | 3.30.161 || TOPORS | 3.30.161 | topoisomerase I binding, arginine/serine-
rich, E3 ubiquitin protein ligase | 0.367545283 | 3.30.161 || TORIB | 3.30.161 | torsin family 1
member B | 0.293724707 | 3.30.161 || TRIM38 | 3.30.161 | tripartite motif containing 38 |
0.229195649 | 3.30.161 || TSEN2 | 3.30.161 | tRNA splicing endonuclease subunit 2 | 0.40337825 |
3.30.161 || TSR1 | 3.30.161 | TSR1, 20S rRNA accumulation, homolog (S. cerevisiae) |
0.231857811 | 3.30.161 || TTC32 | 3.30.161 | tetratricopeptide repeat domain 32 | 0.363074357 |
3.30.161 || UACA | 3.30.161 | uveal autoantigen with coiled-coil domains and ankyrin repeats |
0.246988249 | 3.30.161 || UBAC1 | 3.30.161 | UBA domain containing 1 | 0.214671317 | 3.30.161 ||
UBE2S | 3.30.161 | ubiquitin conjugating enzyme E2S | 0.268642983 | 3.30.161 || UBXN2A |
3.30.161 | UBX domain protein 2A | 0.332342825 | 3.30.161 || USP34 | 3.30.161 | ubiquitin specific
peptidase 34 | 0.440042036 | 3.30.161 || VTIIA | 3.30.161 | vesicle transport through interaction
with t-SNAREs 1A | 0.477960914 | 3.30.161 || WDPCP | 3.30.161 | WD repeat containing planar
cell polarity effector | 0.521484164 | 3.30.161 || WDR92 | 3.30.161 | WD repeat domain 92 |
0.604784359 | 3.30.161 || WTAP | 3.30.161 | Wilms tumor 1 associated protein | 0.324388067 |
3.30.161 || WWP1 | 3.30.161 | WW domain containing E3 ubiquitin protein ligase 1 | 0.267222347 |
3.30.161 || ZGPAT | 3.30.161 | zinc finger CCCH-type and G-patch domain containing |
0.35496962 | 3.30.161 || ZNF136 | 3.30.161 | zinc finger protein 136 | 0.741766055 | 3.30.161 ||
ZNF160 | 3.30.161 | zinc finger protein 160 | 0.472219291 | 3.30.161 || ZNF185 | 3.30.161 | zinc
finger protein 185 (LIM domain) | 0.243878375 | 3.30.161 || ZNF275 | 3.30.161 | zinc finger protein
275 | 0.381879871 | 3.30.161 || ZNF333 | 3.30.161 | zinc finger protein 333 | 1.010036815 | 3.30.161
|| ZNF37A | 3.30.161 | zinc finger protein 37A | 0.341349617 | 3.30.161 || ZNF37BP | 3.30.161 |
zinc finger protein 37B, pseudogene | 1.791939962 | 3.30.161 || ZNF431 | 3.30.161 | zinc finger
protein 431 | 2.274358104 | 3.30.161 || ZNF451 | 3.30.161 | zinc finger protein 451 | 0.287704099 |
3.30.161 || ZNF514 | 3.30.161 | zinc finger protein 514 | 0.975589962 | 3.30.161 || ZNF518A |
3.30.161 | zinc finger protein 518A | 0.505753968 | 3.30.161 || ZNF528 | 3.30.161 | zinc finger
protein 528 | 0.364951731 | 3.30.161 || ZNF551 | 3.30.161 | zinc finger protein 551 | 1.023046204 |
3.30.161 || ZNF585A | 3.30.161 | zinc finger protein 585A | 0.437874075 | 3.30.161 || ZNF587 |
3.30.161 | zinc finger protein 587 | 0.216364592 | 3.30.161 || ZNF587B | 3.30.161 | zinc finger
protein 587B | 0.259307402 | 3.30.161 || ZNF706 | 3.30.161 | zinc finger protein 706 | 0.29526667 |
3.30.161 || ZNF721 | 3.30.161 | zinc finger protein 721 | 0.299909179 | 3.30.161 || ZNF787 |
3.30.161 | zinc finger protein 787 | 0.292078686 | 3.30.161 || AGA | 3.30.162 |
aspartylglucosaminidase | 0.233971109 | 3.30.162 || BMS1P5 | 3.30.162 | BMS1, ribosome
biogenesis factor pseudogene 5 | 0.681368074 | 3.30.162 || CEP152 | 3.30.162 | centrosomal protein
152kDa | 0.260393368 | 3.30.162 || CEP89 | 3.30.162 | centrosomal protein 89kDa | 0.746193621 |
3.30.162 || CXorf21 | 3.30.162 | chromosome X open reading frame 21 | 0.331323197 | 3.30.162 ||
ETHE1 | 3.30.162 | ethylmalonic encephalopathy 1 | 0.447625879 | 3.30.162 || GTF2H3 | 3.30.162 |
general transcription factor IIH subunit 3 | 0.466118302 | 3.30.162 || HOMER3 | 3.30.162 | homer
scaffolding protein 3 | 0.432097501 | 3.30.162 || LOC100506282 | 3.30.162 | uncharacterized
LOC100506282 | 0.967848883 | 3.30.162 || MFSD4B | 3.30.162 | major facilitator superfamily
domain containing 4B | 0.426375024 | 3.30.162 || NPRL2 | 3.30.162 | NPR2-like, GATOR1
complex subunit | 0.259521118 | 3.30.162 || NXPE3 | 3.30.162 | neurexophilin and PC-esterase
domain family member 3 | 0.655944585 | 3.30.162 || RIT1 | 3.30.162 | Ras-like without CAAX 1 |
0.708957957 | 3.30.162 || SLC35D1 | 3.30.162 | solute carrier family 35 member D1 | 0.355639249 |
3.30.162 || SPRY4-IT1 | 3.30.162 | SPRY4 intronic transcript 1 | 0.272385534 | 3.30.162 ||
TRMT10B | 3.30.162 | tRNA methyltransferase 10B | 0.260649423 | 3.30.162 || TTC5 | 3.30.162 |
tetratricopeptide repeat domain 5 | 0.310589861 | 3.30.162 || UBE3D | 3.30.162 | ubiquitin protein
ligase E3D | 0.606740871 | 3.30.162 || UCKL1 | 3.30.162 | uridine-cytidine kinase 1-like 1 |
0.301351666 | 3.30.162 || VHL | 3.30.162 | von Hippel-Lindau tumor suppressor | 0.599904226 |
3.30.162 || ZDHHC16 | 3.30.162 | zinc finger DHHC-type containing 16 | 0.257332465 | 3.30.162 ||
ZNF230 | 3.30.162 | zinc finger protein 230 | 0.531498798 | 3.30.162 || ZNF75A | 3.30.162 | zinc
finger protein 75a | 0.228062974 | 3.30.162 || ZNF850 | 3.30.162 | zinc finger protein 850 |
0.279345017 | 3.30.162 || AAK1 | 3.30.163 | AP2 associated kinase 1 | 0.358812961 | 3.30.163 ||
ABCF2 | 3.30.163 | ATP binding cassette subfamily F member 2 | 0.22823075 | 3.30.163 || BSG |
3.30.163 | basigin (Ok blood group) | 0.316299297 | 3.30.163 || CCM2 | 3.30.163 | CCM2
scaffolding protein | 0.317281828 | 3.30.163 || CCZ1B | 3.30.163 | CCZ1 homolog B, vacuolar
protein trafficking and biogenesis associated | 0.349017772 | 3.30.163 || CRIPT | 3.30.163 | CXXC
repeat containing interactor of PDZ3 domain | 0.421443808 | 3.30.163 || EBLN3 | 3.30.163 |
endogenous Bornavirus-like nucleoprotein 3 | 0.259890806 | 3.30.163 || GPATCH2L | 3.30.163 | G-
patch domain containing 2 like | 1.960179904 | 3.30.163 || H6PD | 3.30.163 | hexose-6-phosphate
dehydrogenase (glucose 1-dehydrogenase) | 0.420292436 | 3.30.163 || NOP10 | 3.30.163 | NOP10
ribonucleoprotein | 0.23502896 | 3.30.163 || NPTN-IT1 | 3.30.163 | NPTN intronic transcript 1 |
0.645011649 | 3.30.163 || PPHLN1 | 3.30.163 | periphilin 1 | 0.254201029 | 3.30.163 || RABEP1 |
3.30.163 | rabaptin, RAB GTPase binding effector protein 1 | 0.247033339 | 3.30.163 || SCO1 |
3.30.163 | SCO1 cytochrome c oxidase assembly protein | 0.408620694 | 3.30.163 || SMO | 3.30.163
| smoothened, frizzled class receptor | 0.627909031 | 3.30.163 || SSNA1 | 3.30.163 | Sjogren
syndrome nuclear autoantigen 1 | 0.228474379 | 3.30.163 || TMEM209 | 3.30.163 | transmembrane
protein 209 | 0.46025373 | 3.30.163 || TRA2A | 3.30.163 | transformer 2 alpha homolog
(Drosophila) | 0.704028796 | 3.30.163 || TXNDC17 | 3.30.163 | thioredoxin domain containing 17 |
0.220925814 | 3.30.163 || ZNF669 | 3.30.163 | zinc finger protein 669 | 0.390451114 | 3.30.163 ||
AASS | 3.30.164 | aminoadipate-semialdehyde synthase | 1.282105419 | 3.30.164 || ARIH2 |
3.30.164 | ariadne RBR E3 ubiquitin protein ligase 2 | 0.368630654 | 3.30.164 || BORCS6 | 3.30.164
| BLOC-1 related complex subunit 6 | 0.253484607 | 3.30.164 || C12orf43 | 3.30.164 | chromosome
12 open reading frame 43 | 0.606791983 | 3.30.164 || C20orf194 | 3.30.164 | chromosome 20 open
reading frame 194 | 0.496257029 | 3.30.164 || ELAVL3 | 3.30.164 | ELAV like neuron-specific
RNA binding protein 3 | 0.538596786 | 3.30.164 || FBXL14 | 3.30.164 | F-box and leucine-rich
repeat protein 14 | 0.24496978 | 3.30.164 || HOXC9 | 3.30.164 | homeobox C9 | 0.439238775 |
3.30.164 || MAPRE3 | 3.30.164 | microtubule associated protein RP/EB family member 3 |
0.357711959 | 3.30.164 || MBIP | 3.30.164 | MAP3K12 binding inhibitory protein 1 | 0.707044418 |
3.30.164 || MTRFIL | 3.30.164 | mitochondrial translational release factor 1 like | 0.512985038 |
3.30.164 || NMT1 | 3.30.164 | N-myristoyltransferase 1 | 0.344492238 | 3.30.164 || NPEPL1 |
3.30.164 | aminopeptidase-like 1 | 0.505830032 | 3.30.164 || NRIP1 | 3.30.164 | nuclear receptor
interacting protein 1 | 0.540479072 | 3.30.164 || NUMBL | 3.30.164 | numb homolog (Drosophila)-
like | 0.452337826 | 3.30.164 || PALM2 | 3.30.164 | paralemmin 2 | 0.624064675 | 3.30.164 ||
RAPH1 | 3.30.164 | Ras association (RalGDS/AF-6) and pleckstrin homology domains 1 |
0.246647221 | 3.30.164 || RPRD2 | 3.30.164 | regulation of nuclear pre-mRNA domain containing 2
| 0.355487833 | 3.30.164 || SEC22A | 3.30.164 | SEC22 homolog A, vesicle trafficking protein |
0.390611834 | 3.30.164 || SEZ6L2 | 3.30.164 | seizure related 6 homolog (mouse)-like 2 |
0.372348623 | 3.30.164 || STXBP5 | 3.30.164 | syntaxin binding protein 5 | 1.089528566 | 3.30.164
|| TBC1D32 | 3.30.164 | TBC1 domain family member 32 | 0.324652475 | 3.30.164 || TMEM67 |
3.30.164 | transmembrane protein 67 | 0.677453744 | 3.30.164 || UBN2 | 3.30.164 | ubinuclein 2 |
0.436643149 | 3.30.164 || VAMP2 | 3.30.164 | vesicle associated membrane protein 2 | 0.389730645
| 3.30.164 || VGLL4 | 3.30.164 | vestigial like family member 4 | 0.273340825 | 3.30.164 || WASF1 |
3.30.164 | WAS protein family member 1 | 0.455656022 | 3.30.164 || ZNF785 | 3.30.164 | zinc
finger protein 785 | 2.268123854 | 3.30.164 || ADAMTS17 | 3.31 | ADAM metallopeptidase with
thrombospondin type 1 motif 17 | 0.399201568 | - || AHDC1 | 3.31 | AT-hook DNA binding motif
containing 1 | 0.501331107 | - || ARMC5 | 3.31 | armadillo repeat containing 5 | 0.428724173 | - ||
ATHL1 | 3.31 | ATHI, acid trehalase-like 1 (yeast) | 0.249640217 | - || DGCR14 | 3.31 | DiGeorge
syndrome critical region gene 14 | 0.279565174 | - || EML2 | 3.31 | echinoderm microtubule
associated protein like 2 | 0.429898141 | - || FMN2 | 3.31 | formin 2 | 0.475511702 | - || GLIS3 | 3.31
| GLIS family zinc finger 3 | 1.01236834 | - || GLTSCR2 | 3.31 | glioma tumor suppressor candidate
region gene 2 | 0.494146367 | - || HMCN2 | 3.31 | hemicentin 2 | 0.248366682 | - || IRF4 | 3.31 |
interferon regulatory factor 4 | 0.584033475 | - || JSRP1 | 3.31 | junctional sarcoplasmic reticulum
protein 1 | 0.693162624 | - || LIMD2 | 3.31 | LIM domain containing 2 | 0.223954551 | - || LRRC16B
| 3.31 | leucine rich repeat containing 16B | 0.350578075 | - || MCPH1 | 3.31 | microcephalin 1 |
0.244538247 | - || MYBL1 | 3.31 | MYB proto-oncogene like 1 | 0.576729686 | - || NBAT1 | 3.31 |
neuroblastoma associated transcript 1 | 0.508058663 | - || PAX5 | 3.31 | paired box 5 | 0.62551336 | -
|| PDE3B | 3.31 | phosphodiesterase 3B | 0.385717865 | - || PDE7B | 3.31 | phosphodiesterase 7B |
0.88226372 | - || PLCD3 | 3.31 | phospholipase C delta 3 | 0.470611566 | - || PRKD2 | 3.31 | protein
kinase D2 | 1.164808234 | - || REXO1 | 3.31 | REX1, RNA exonuclease 1 homolog | 0.592885734 | -
|| SAMD5 | 3.31 | sterile alpha motif domain containing 5 | 0.931112195 | - || SLC4A9 | 3.31 | solute
carrier family 4 member 9 | 0.372045141 | - || SPATA6L | 3.31 | spermatogenesis associated 6 like |
0.331836242 | - || TBC1D10C | 3.31 | TBC1 domain family member 10C | 0.350887391 | - ||
TBXA2R | 3.31 | thromboxane A2 receptor | 0.409684751 | - || TCF15 | 3.31 | transcription factor 15
(basic helix-loop-helix) | 0.330400545 | - || TRO | 3.31 | trophinin | 0.297771396 | - || ZXDB | 3.31 |
zinc finger, X-linked, duplicated B | 0.217360817 | - || AK2 | 3.33 | adenylate kinase 2
0.327779045 | 3.33.178 | AMFR | 3.33 | autocrine motility factor receptor, E3 ubiquitin protein
ligase | 3.041255337 | 3.33.178 || AP1G2 | 3.33 | adaptor related protein complex 1 gamma 2
subunit | 0.267688081 | - || ATAD2 | 3.33 | ATPase family, AAA domain containing 2 |
0.245118807 | - || ATF7IP2 | 3.33 | activating transcription factor 7 interacting protein 2 |
0.225243445 | - || ATOX1 | 3.33 | antioxidant 1 copper chaperone | 0.22425753 | 3.33.178 ||
ATP6VIE1 | 3.33 | ATPase H+ transporting VI subunit El | 0.238647016 | 3.33.178 | ATXN7 |
3.33 | ataxin 7 | 0.351234243 | - || AURKAIP1 | 3.33 | aurora kinase A interacting protein 1 |
0.224613317 | 3.33.178 | BCAP31 | 3.33 | B-cell receptor-associated protein 31 | 0.276306527 | - ||
C11orf80 | 3.33 | chromosome 11 open reading frame 80 | 0.684775027 | 3.33.178 | CA5BP1 | 3.33
| carbonic anhydrase VB pseudogene 1 | 0.711806365 | 3.33.178 || CARF | 3.33 | calcium responsive
transcription factor | 0.439071516 | - || CCDC28B | 3.33 | coiled-coil domain containing 28B |
0.338314322 | 3.33.178 || CLPP | 3.33 | caseinolytic mitochondrial matrix peptidase proteolytic
subunit | 0.275667193 | 3.33.178 || CYLD | 3.33 | CYLD lysine 63 deubiquitinase | 0.566163172 | -
|| DCAF8 | 3.33 | DDB1 and CUL4 associated factor 8 | 0.530852721 | 3.33.178 || DNAJA1 | 3.33 |
DnaJ heat shock protein family (Hsp40) member Al | 0.268873868 | 3.33.178 || ECSIT | 3.33 |
ECSIT signalling integrator | 0.352705202 | 3.33.178 || FAM118B | 3.33 | family with sequence
similarity 118 member B | 0.238839634 | 3.33.178 || FAM214A | 3.33 | family with sequence
similarity 214 member A | 0.302710804 | - || FAM21C | 3.33 | family with sequence similarity 21
member C | 0.27369566 | - || FBXL18 | 3.33 | F-box and leucine-rich repeat protein 18 |
0.232762532 | - || FBXO22 | 3.33 | F-box protein 22 | 1.788767882 | 3.33.178 || GATAD1 | 3.33 |
GATA zinc finger domain containing 1 | 0.356910926 | - || GLIPR2 | 3.33 | GLI pathogenesis-
related 2 | 0.665857308 | - || GPR155 | 3.33 | G protein-coupled receptor 155 | 1.604927816 |
3.33.178 || GSTK1 | 3.33 | glutathione S-transferase kappa 1 | 0.220726129 | - || GUSB | 3.33 |
glucuronidase, beta | 0.280321806 | - || HAGH | 3.33 | hydroxyacylglutathione hydrolase
0.256994681 | 3.33.178 || HELLS | 3.33 | helicase, lymphoid-specific | 0.263937318 | - || HN1 | 3.33
| hematological and neurological expressed 1 | 0.354875566 | 3.33.178 || HSPH1 | 3.33 | heat shock
protein family H (Hsp110) member 1 | 0.234026353 | 3.33.178 || ILF3-AS1 | 3.33 | ILF3 antisense
RNA 1 (head to head) | 0.307890642 | - || KCNE3 | 3.33 | potassium voltage-gated channel
subfamily E regulatory subunit 3 | 0.271632127 | - || LPIN1 | 3.33 | lipin 1 | 0.326521724 | - ||
LRPAP1 | 3.33 | LDL receptor related protein associated protein 1 | 0.233469226 | - || MAP3K8 |
3.33 | mitogen-activated protein kinase kinase kinase 8 | 1.107677445 | 3.33.178 || MCM8 | 3.33 |
minichromosome maintenance 8 homologous recombination repair factor | 0.540601513 | - ||
MIEN1 | 3.33 | migration and invasion enhancer 1 | 0.239734321 | 3.33.178 || MSRB1 | 3.33 |
methionine sulfoxide reductase B1 | 0.415707406 | 3.33.178 || N4BP2 | 3.33 | NEDD4 binding
protein 2 | 0.729558853 | - || NCBP1 | 3.33 | nuclear cap binding protein subunit 1 | 0.222253372 |
3.33.178 || NUPR1 | 3.33 | nuclear protein 1, transcriptional regulator | 0.445955944 | - || PLXNC1 |
3.33 | plexin C1 | 0.656119334 | 3.33.178 || POLR2J4 | 3.33 | polymerase (RNA) II subunit J4,
pseudogene | 0.896981432 | 3.33.178 || PRPF38B | 3.33 | pre-mRNA processing factor 38B |
1.067536004 | 3.33.178 || PUDP | 3.33 | pseudouridine 5′-phosphatase | 0.227580038 | - ||
RAPIGDS1 | 3.33 | Rap1 GTPase-GDP dissociation stimulator 1 | 0.232134376 | 3.33.178 || RBBP6
| 3.33 | retinoblastoma binding protein 6 | 0.247349278 | - || RGS10 | 3.33 | regulator of G-protein
signaling 10 | 0.218425273 | 3.33.178 || RHNO1 | 3.33 | RAD9-HUS1-RADI interacting nuclear
orphan 1 | 0.743633056 | 3.33.178 || RNF213 | 3.33 | ring finger protein 213 | 0.678545516 |
3.33.178 || SCLT1 | 3.33 | sodium channel and clathrin linker 1 | 0.265780406 | - || SMYD2 | 3.33 |
SET and MYND domain containing 2 | 0.242059162 | 3.33.178 || SYTL3 | 3.33 | synaptotagmin like
3 | 2.021400017 | 3.33.178 || SZRD1 | 3.33 | SUZ RNA binding domain containing 1 | 0.220013406
| - || TALDO1 | 3.33 | transaldolase 1 | 0.304590832 | - || TEFM | 3.33 | transcription elongation
factor, mitochondrial | 0.957503241 | 3.33.178 || THBD | 3.33 | thrombomodulin | 0.701206969 |
3.33.178 || TKT | 3.33 | transketolase | 0.402606279 | - || TMEM11 | 3.33 | transmembrane protein
11 | 0.2704814 | 3.33.178 || TMEM140 | 3.33 | transmembrane protein 140 | 0.631231263 | - ||
TMEM141 | 3.33 | transmembrane protein 141 | 0.251271929 | - || TP53RK | 3.33 | TP53 regulating
kinase | 0.340157462 | 3.33.178 || TRAPPC10 | 3.33 | trafficking protein particle complex 10 |
1.066633667 | 3.33.178 || TUSC2 | 3.33 | tumor suppressor candidate 2 | 0.283715013 | 3.33.178 ||
UNK | 3.33 | unkempt family zinc finger | 0.596736094 | 3.33.178 || UVSSA | 3.33 | UV stimulated
scaffold protein A | 0.672393502 | 3.33.178 || VASH1 | 3.33 | vasohibin 1 | 1.785854403 | 3.33.178 ||
ZCCHC7 | 3.33 | zinc finger CCHC-type containing 7 | 0.638314648 | 3.33.178 || ZMAT3 | 3.33 |
zinc finger matrin-type 3 | 0.354623939 | - || ZNF224 | 3.33 | zinc finger protein 224 | 0.87763591 |
3.33.178 || ZNF568 | 3.33 | zinc finger protein 568 | 0.236065721 | - || ZXDC | 3.33 | ZXD family
zinc finger C | 0.86392643 | 3.33.178 || AK2 | 3.33.178 | adenylate kinase 2 | 0.327779045 |
3.33.178 || AMFR | 3.33.178 | autocrine motility factor receptor, E3 ubiquitin protein ligase |
3.041255337 | 3.33.178 || ATOX1 | 3.33.178 | antioxidant 1 copper chaperone | 0.22425753 |
3.33.178 || ATP6VIE1 | 3.33.178 | ATPase H+ transporting VI subunit El | 0.238647016 | 3.33.178
|| AURKAIP1 | 3.33.178 | aurora kinase A interacting protein 1 | 0.224613317 | 3.33.178 || C11orf80
| 3.33.178 | chromosome 11 open reading frame 80 | 0.684775027 | 3.33.178 || CA5BP1 | 3.33.178
carbonic anhydrase VB pseudogene 1 | 0.711806365 | 3.33.178 || CCDC28B | 3.33.178 | coiled-coil
domain containing 28B | 0.338314322 | 3.33.178 | CLPP | 3.33.178 || caseinolytic mitochondrial
matrix peptidase proteolytic subunit | 0.275667193 | 3.33.178 || DCAF8 | 3.33.178 | DDB1 and
CUL4 associated factor 8 | 0.530852721 | 3.33.178 || DNAJA1 | 3.33.178 | DnaJ heat shock protein
family (Hsp40) member Al | 0.268873868 | 3.33.178 || ECSIT | 3.33.178 | ECSIT signalling
integrator | 0.352705202 | 3.33.178 || FAM118B | 3.33.178 | family with sequence similarity 118
member B | 0.238839634 | 3.33.178 || FBXO22 | 3.33.178 | F-box protein 22 | 1.788767882 |
3.33.178 || GPR155 | 3.33.178 | G protein-coupled receptor 155 | 1.604927816 | 3.33.178 || HAGH |
3.33.178 | hydroxyacylglutathione hydrolase | 0.256994681 | 3.33.178 || HN1 | 3.33.178 |
hematological and neurological expressed 1 | 0.354875566 | 3.33.178 || HSPH1 | 3.33.178 | heat
shock protein family H (Hsp110) member 1 | 0.234026353 | 3.33.178 || MAP3K8 | 3.33.178 |
mitogen-activated protein kinase kinase kinase 8 | 1.107677445 | 3.33.178 || MIEN1 | 3.33.178 |
migration and invasion enhancer 1 | 0.239734321 | 3.33.178 || MSRB1 | 3.33.178 | methionine
sulfoxide reductase B1 | 0.415707406 | 3.33.178 || NCBP1 | 3.33.178 | nuclear cap binding protein
subunit 1 | 0.222253372 | 3.33.178 || PLXNC1 | 3.33.178 | plexin C1 | 0.656119334 | 3.33.178 ||
POLR2J4 | 3.33.178 | polymerase (RNA) II subunit J4, pseudogene | 0.896981432 | 3.33.178 ||
PRPF38B | 3.33.178 | pre-mRNA processing factor 38B | 1.067536004 | 3.33.178 || RAPIGDS1 |
3.33.178 | Rap1 GTPase-GDP dissociation stimulator 1 | 0.232134376 | 3.33.178 || RGS10 |
3.33.178 | regulator of G-protein signaling 10 | 0.218425273 | 3.33.178 || RHNO1 | 3.33.178 |
RAD9-HUS1-RADI interacting nuclear orphan 1 | 0.743633056 | 3.33.178 || RNF213 | 3.33.178 |
ring finger protein 213 | 0.678545516 | 3.33.178 || SMYD2 | 3.33.178 | SET and MYND domain
containing 2 | 0.242059162 | 3.33.178 || SYTL3 | 3.33.178 | synaptotagmin like 3 | 2.021400017 |
3.33.178 || TEFM | 3.33.178 | transcription elongation factor, mitochondrial | 0.957503241 |
3.33.178 || THBD | 3.33.178 | thrombomodulin | 0.701206969 | 3.33.178 || TMEM11 | 3.33.178 |
transmembrane protein 11 | 0.2704814 | 3.33.178 || TP53RK | 3.33.178 | TP53 regulating kinase |
0.340157462 | 3.33.178 || TRAPPC10 | 3.33.178 | trafficking protein particle complex 10 |
1.066633667 | 3.33.178 || TUSC2 | 3.33.178 | tumor suppressor candidate 2 | 0.283715013 |
3.33.178 || UNK | 3.33.178 | unkempt family zinc finger | 0.596736094 | 3.33.178 || UVSSA |
3.33.178 | UV stimulated scaffold protein A | 0.672393502 | 3.33.178 || VASH1 | 3.33.178 |
vasohibin 1 | 1.785854403 | 3.33.178 || ZCCHC7 | 3.33.178 | zinc finger CCHC-type containing 7 |
0.638314648 | 3.33.178 || ZNF224 | 3.33.178 | zinc finger protein 224 | 0.87763591 | 3.33.178 ||
ZXDC | 3.33.178 | ZXD family zinc finger C | 0.86392643 | 3.33.178 || ABHD18 | 3.34 |
abhydrolase domain containing 18 | 0.347263669 | 3.34.183 || ACAP1 | 3.34 | ArfGAP with coiled-
coil, ankyrin repeat and PH domains 1 | 0.797943451 | 3.34.181 || ACSL5 | 3.34 | acyl-CoA
synthetase long-chain family member 5 | 0.518299994 | 3.34.183 || ADAP2 | 3.34 | ArfGAP with
dual PH domains 2 | 0.706325016 | 3.34.183| | AGO2 | 3.34 | argonaute 2, RISC catalytic
component | 0.494685271 | 3.34.183 || AKAP7 | 3.34 | A-kinase anchoring protein 7 | 0.308544402 |
3.34.181 || ALDH16A1 | 3.34 | aldehyde dehydrogenase 16 family member Al | 0.358801572 |
3.34.181 || ANKRD13D | 3.34 | ankyrin repeat domain 13 family member D | 0.496209116 |
3.34.183 || AOAH | 3.34 | acyloxyacyl hydrolase | 0.788599525 | 3.34.182 || APOL3 | 3.34 |
apolipoprotein L3 | 0.444787301 | 3.34.181 || AQP3 | 3.34 | aquaporin 3 (Gill blood group) |
0.669230649 | 3.34.181 || ARL10 | 3.34 | ADP ribosylation factor like GTPase 10 | 0.274793381 |
3.34.181 || ARMC6 | 3.34 | armadillo repeat containing 6 | 0.292418867 | 3.34.181 || ARMCX3 |
3.34 | armadillo repeat containing, X-linked 3 | 0.344611292 | 3.34.181 || ARNTL | 3.34 | aryl
hydrocarbon receptor nuclear translocator like | 0.261599179 | 3.34.182 || ATF3 | 3.34 | activating
transcription factor 3 | 3.058885398 | 3.34.181 || B3GAT3 | 3.34 | beta-1,3-glucuronyltransferase 3 |
0.362118456 | 3.34.181 || BMP8B | 3.34 | bone morphogenetic protein 8b | 0.401399873 | 3.34.183 ||
BTN3A3 | 3.34 | butyrophilin subfamily 3 member A3 | 0.359570298 | 3.34.181 || C12orf66 | 3.34 |
chromosome 12 open reading frame 66 | 1.186714159 | 3.34.181 || C14orf28 | 3.34 | chromosome 14
open reading frame 28 | 0.351876729 | 3.34.181 || C1QTNF7 | 3.34 | Clq and tumor necrosis factor
related protein 7 | 2.282375332 | 3.34.181 || C5orf56 | 3.34 | chromosome 5 open reading frame 56 |
0.401852251 | 3.34.183 || C7orf73 | 3.34 | chromosome 7 open reading frame 73 | 0.253127871 |
3.34.182 || CALM3 | 3.34 | calmodulin 3 (phosphorylase kinase, delta) | 0.505532715 | 3.34.181 |
CAMKK2 | 3.34 | calcium/calmodulin-dependent protein kinase kinase 2 | 0.522766945 | 3.34.182 ||
CASD1 | 3.34 | CASI domain containing 1 | 0.997233305 | 3.34.181 || CBFA2T2 | 3.34 | core-
binding factor, runt domain, alpha subunit 2; translocated to, 2 | 0.686453527 | 3.34.181 || CBX5 |
3.34 | chromobox 5 | 0.943629254 | 3.34.183 || CCNL1 | 3.34 | cyclin L1 | 0.718233035 | 3.34.181 ||
CD180 | 3.34 | CD180 molecule | 0.577642978 | 3.34.182 || CDKNIA | 3.34 | cyclin-dependent
kinase inhibitor 1A | 0.293563749 | 3.34.181 || CEP85L | 3.34 | centrosomal protein 85kDa-like |
0.426902691 | 3.34.181 || CFLAR | 3.34 | CASP8 and FADD like apoptosis regulator | 1.01380616 |
3.34.183 || CHMP2B | 3.34 | charged multivesicular body protein 2B | 0.226092619 | 3.34.181 ||
CNNM3 | 3.34 | cyclin and CBS domain divalent metal cation transport mediator 3 | 0.561866781 |
3.34.181 || CRAMP1 | 3.34 | cramped chromatin regulator homolog 1 | 0.279416937 | 3.34.183 ||
CSPG4 | 3.34 | chondroitin sulfate proteoglycan 4 | 0.541725567 | 3.34.182 || CTSZ | 3.34 |
cathepsin Z | 1.961412647 | 3.34.181 || CUEDC1 | 3.34 | CUE domain containing 1 | 0.249274597 |
3.34.181 || CXADR | 3.34 | coxsackie virus and adenovirus receptor | 1.789764956 | 3.34.183 ||
CXXC5 | 3.34 | CXXC finger protein 5 | 0.47215513 | 3.34.181 || DIS3L2 | 3.34 | DIS3 like 3′-5′
exoribonuclease 2 | 0.960816554 | 3.34.181 || DLEU2 | 3.34 | deleted in lymphocytic leukemia 2
(non-protein coding) | 1.814822257 | 3.34.183 || DNAJB14 | 3.34 | DnaJ heat shock protein family
(Hsp40) member B14 | 0.355043803 | 3.34.181 || DTWD1 | 3.34 | DTW domain containing 1 |
0.617079271 | 3.34.181 || EFHC1 | 3.34 | EF-hand domain (C-terminal) containing 1 | 0.400427115 |
3.34.181 || EGFR | 3.34 | epidermal growth factor receptor | 2.045949226 | 3.34.181 || EIF1AX |
3.34 | eukaryotic translation initiation factor 1A, X-linked | 0.416610933 | 3.34.182 || ENPP7 | 3.34 |
ectonucleotide pyrophosphatase/phosphodiesterase 7 | 0.278869659 | 3.34.181 || ERCC6L2 | 3.34 |
excision repair cross-complementation group 6 like 2 | 0.285980254 | 3.34.181 || ETV1 | 3.34 | ETS
variant 1 | 0.912856796 | 3.34.181 || FAM3A | 3.34 | family with sequence similarity 3 member A |
0.326794539 | 3.34.181 || FAM58A | 3.34 | family with sequence similarity 58 member A |
0.295626083 | 3.34.181 || FAM63B | 3.34 | family with sequence similarity 63 member B |
0.728481399 | 3.34.181 || FBXO21 | 3.34 | F-box protein 21 | 0.506755406 | 3.34.181 || FGD4 | 3.34
| FYVE, RhoGEF and PH domain containing 4 | 0.484661461 | 3.34.181 || FNIP2 | 3.34 | folliculin
interacting protein 2 | 0.471294846 | 3.34.181 || FRMD3 | 3.34 | FERM domain containing 3 |
1.477940515 | 3.34.181 || FUBP3 | 3.34 | far upstream element (FUSE) binding protein 3 |
0.241847541 | 3.34.182 || FXR2 | 3.34 | FMRI autosomal homolog 2 | 0.231085202 | 3.34.181 ||
GALK2 | 3.34 | galactokinase 2 | 0.343011992 | 3.34.181 || GAPLINC | 3.34 | gastric
adenocarcinoma associated, positive CD44 regulator, long intergenic non-coding RNA |
0.443804638 | 3.34.182 || GAS7 | 3.34 | growth arrest specific 7 | 0.543059417 | 3.34.182 ||
GEMIN2 | 3.34 | gem nuclear organelle associated protein 2 | 0.919606402 | 3.34.181 || GINM1 |
3.34 | glycoprotein integral membrane 1 | 0.222152336 | 3.34.181 || GOSR1 | 3.34 | golgi SNAP
receptor complex member 1 | 0.293067756 | 3.34.181 || GOSR2 | 3.34 | golgi SNAP receptor
complex member 2 | 0.316869064 | 3.34.181 || GPR137 | 3.34 | G protein-coupled receptor 137 |
0.366510951 | 3.34.181 || GSE1 | 3.34 | Gsel coiled-coil protein | 0.856456124 | 3.34.182 || GSS |
3.34 | glutathione synthetase | 0.3066072 | 3.34.181 || HCFC2 | 3.34 | host cell factor C2 |
0.503475927 | 3.34.181 || HERC5 | 3.34 | HECT and RLD domain containing E3 ubiquitin protein
ligase 5 | 0.538314901 | 3.34.181 || HMGXB3 | 3.34 | HMG-box containing 3 | 0.472704277 |
3.34.181 || HSD11B1 | 3.34 | hydroxysteroid (11-beta) dehydrogenase 1 | 0.37414609 | 3.34.183|
IFT74 | 3.34 | intraflagellar transport 74 | 0.768727706 | 3.34.181 || IL18 | 3.34 | interleukin 18 |
0.717477945 | 3.34.181 || IL34 | 3.34 | interleukin 34 | 0.311738031 | 3.34.181 || ING3 | 3.34 |
inhibitor of growth family member 3 | 0.217453446 | 3.34.181 || IPP | 3.34 | intracisternal A
particle-promoted polypeptide | 0.375329701 | 3.34.181 || IRF2BP2 | 3.34 | interferon regulatory
factor 2 binding protein 2 | 0.349979352 | 3.34.182 || ITPRIPL2 | 3.34 | inositol 1,4,5-trisphosphate
receptor interacting protein-like 2 | 0.321902861 | 3.34.181 || KCNAB1 | 3.34 | potassium voltage-
gated channel subfamily A member regulatory beta subunit 1 | 2.163453915 | 3.34.181 || KDSR |
3.34 | 3-ketodihydrosphingosine reductase | 0.255843924 | 3.34.181 || KIAA0040 | 3.34 | KIAA0040
| 0.299569126 | 3.34.183 || KIAA1715 | 3.34 | KIAA1715 | 0.554843041 | 3.34.181 || KIAA2026 |
3.34 | KIAA2026 | 0.340138639 | 3.34.181 || KLF6 | 3.34 | Kruppel-like factor 6 | 0.335741053 |
3.34.181 || KMT2E | 3.34 | lysine methyltransferase 2E | 0.391708808 | 3.34.181 || KMT5B | 3.34 |
lysine methyltransferase 5B | 0.320865943 | 3.34.181 || LITD1 | 3.34 | LINE-1 type transposase
domain containing 1 | 0.295829028 | 3.34.183 || LATS2 | 3.34 | large tumor suppressor kinase 2 |
0.416031539 | 3.34.182 || LGALS2 | 3.34 | lectin, galactoside-binding, soluble, 2 | 0.899211275 |
3.34.183 || LIN52 | 3.34 | lin-52 DREAM MuvB core complex component | 0.27931529 | 3.34.181 ||
LITAF | 3.34 | lipopolysaccharide-induced TNF factor | 0.369216561 | 3.34.183 || LMLN | 3.34 |
leishmanolysin like peptidase | 0.257046037 | 3.34.181 || LOC101927204 | 3.34 | uncharacterized
LOC101927204 | 0.772510645 | 3.34.181 || LOC102724094 | 3.34 | uncharacterized
LOC102724094 | 0.548308199 | 3.34.181 || LRRC46 | 3.34 | leucine rich repeat containing 46 |
0.451347236 | 3.34.182 || LY96 | 3.34 | lymphocyte antigen 96 | 0.314354256 | 3.34.182 || LYAR |
3.34 | Lyl antibody reactive | 0.220128558 | 3.34.183 || MAGI3 | 3.34 | membrane associated
guanylate kinase, WW and PDZ domain containing 3 | 0.277852189 | 3.34.181 || MAML2 | 3.34 |
mastermind like transcriptional coactivator 2 | 0.226580944 | 3.34.181 || MANF | 3.34 |
mesencephalic astrocyte derived neurotrophic factor | 0.307536561 | 3.34.182 || MBD4 | 3.34 |
methyl-CpG binding domain 4 DNA glycosylase | 0.249626556 | 3.34.183 || MBNL3 | 3.34 |
muscleblind like splicing regulator 3 | 0.264231685 | 3.34.181 || MCL1 | 3.34 | myeloid cell
leukemia 1 | 0.289826855 | 3.34.181 || MCOLN1 | 3.34 | mucolipin 1 | 0.352743785 | 3.34.181 ||
MDGA1 | 3.34 | MAM domain containing glycosylphosphatidylinositol anchor 1 | 0.272317702 |
3.34.182 || MDM4 | 3.34 | MDM4, p53 regulator | 0.260834197 | 3.34.181 || MFSD12 | 3.34 | major
facilitator superfamily domain containing 12 | 0.611241382 | 3.34.181 || MPHOSPH8 | 3.34 | M-
phase phosphoprotein 8 | 0.453451819 | 3.34.182 || MPHOSPH9 | 3.34 | M-phase phosphoprotein 9 |
0.789933229 | 3.34.183 || MSANTD2 | 3.34 | Myb/SANT DNA binding domain containing 2 |
0.713568311 | 3.34.181 || MTAP | 3.34 | methylthioadenosine phosphorylase | 0.720927807 |
3.34.182 || MTHFR | 3.34 | methylenetetrahydrofolate reductase (NAD(P)H) | 0.714030773 |
3.34.183 || MTPAP | 3.34 | mitochondrial poly(A) polymerase | 0.375695773 | 3.34.181 || MUC5AC
| 3.34 | mucin 5AC, oligomeric mucus/gel-forming | 1.13884726 | 3.34.181 || MYCBP2 | 3.34 |
MYC binding protein 2, E3 ubiquitin protein ligase | 0.458305109 | 3.34.181 || MYDGF | 3.34
myeloid-derived growth factor | 0.324808966 | 3.34.182 || MYLIP | 3.34 | myosin regulatory light
chain interacting protein | 0.285629863 | 3.34.181 || NAGK | 3.34 | N-acetylglucosamine kinase |
0.362682316 | 3.34.181 || NBEAL1 | 3.34 | neurobeachin like 1 | 1.746272399 | 3.34.182 || NFIL3 |
3.34 | nuclear factor, interleukin 3 regulated | 0.387157537 | 3.34.183 || NPHP3 | 3.34 |
nephronophthisis 3 (adolescent) | 1.109658891 | 3.34.181 || NR1D2 | 3.34 | nuclear receptor
subfamily 1 group D member 2 | 2.339118073 | 3.34.182 || NSD1 | 3.34 | nuclear receptor binding
SET domain protein 1 | 0.484225394 | 3.34.183 || ODF3B | 3.34 | outer dense fiber of sperm tails 3B
| 0.571785467 | 3.34.181 || OGG1 | 3.34 | 8-oxoguanine DNA glycosylase | 0.730181493 | 3.34.181
|| ORC2 | 3.34 | origin recognition complex subunit 2 | 0.252480341 | 3.34.183 || OTUD6B-AS1 |
3.34 | OTUD6B antisense RNA 1 (head to head) | 0.34976707 | 3.34.181 || PCSK6 | 3.34 |
proprotein convertase subtilisin/kexin type 6 | 3.160999748 | 3.34.183 || PCYOX1 | 3.34 |
prenylcysteine oxidase 1 | 0.329734083 | 3.34.181 || PDPR | 3.34 | pyruvate dehydrogenase
phosphatase regulatory subunit | 0.830063041 | 3.34.182 || PER2 | 3.34 | period circadian clock 2 |
0.972020801 | 3.34.182 || PER3 | 3.34 | period circadian clock 3 | 0.903397282 | 3.34.182 || PHAX |
3.34 | phosphorylated adaptor for RNA export | 0.2443868 | 3.34.181 || PIAS2 | 3.34 | protein
inhibitor of activated STAT 2 | 0.485873938 | 3.34.181 || PIM3 | 3.34 | Pim-3 proto-oncogene,
serine/threonine kinase | 0.3316686 | 3.34.183 || PLEKHM3 | 3.34 | pleckstrin homology domain
containing M3 | 0.436968042 | 3.34.181 || PM20D2 | 3.34 | peptidase M20 domain containing 2 |
0.779598912 | 3.34.181 || PNISR | 3.34 | PNN-interacting serine/arginine-rich protein | 0.767471395
| 3.34.181 || POSTN | 3.34 | periostin, osteoblast specific factor | 1.771653828 | 3.34.183 || POU6F1
| 3.34 | POU class 6 homeobox 1 | 0.344882068 | 3.34.181 || PPP1R15A | 3.34 | protein phosphatase
1 regulatory subunit 15A | 0.777418995 | 3.34.181 || PPP1R3E | 3.34 | protein phosphatase 1
regulatory subunit 3E | 0.854250805 | 3.34.181 || PPTC7 | 3.34 | PTC7 protein phosphatase homolog
| 0.287690259 | 3.34.182 || PRKCI | 3.34 | protein kinase C iota | 0.341965274 | 3.34.181 || PRX |
3.34 | periaxin | 0.244877377 | 3.34.181 || PSD3 | 3.34 | pleckstrin and Sec7 domain containing 3 |
0.985521347 | 3.34.181 || PTER | 3.34 | phosphotriesterase related | 0.685036329 | 3.34.181 ||
RAB3GAP1 | 3.34 | RAB3 GTPase activating protein catalytic subunit 1 | 0.24367303 | 3.34.181 ||
RAB5C | 3.34 | RAB5C, member RAS oncogene family | 0.708900601 | 3.34.181 || RAD51-AS1 |
3.34 | RAD51 antisense RNA 1 (head to head) | 1.117574449 | 3.34.181 || RASAL2 | 3.34 | RAS
protein activator like 2 | 0.276634505 | 3.34.181 || RB1 | 3.34 | retinoblastoma 1 | 0.221328701 |
3.34.181 || RBCK1 | 3.34 | RANBP2-type and C3HC4-type zinc finger containing 1 | 0.229269756 |
3.34.181 || RBM12B | 3.34 | RNA binding motif protein 12B | 0.523219204 | 3.34.183 || RBM25 |
3.34 | RNA binding motif protein 25 | 0.338114959 | 3.34.181 || RBM26 | 3.34 | RNA binding motif
protein 26 | 0.368093556 | 3.34.181 || RBM41 | 3.34 | RNA binding motif protein 41 | 0.867318437 |
3.34.181 || RBM6 | 3.34 | RNA binding motif protein 6 | 0.897328782 | 3.34.183 || RCBTB1 | 3.34 |
RCC1 and BTB domain containing protein 1 | 0.249974114 | 3.34.181 || RNF170 | 3.34 | ring finger
protein 170 | 0.290631795 | 3.34.181 || RPL32P3 | 3.34 | ribosomal protein L32 pseudogene 3 |
0.988371146 | 3.34.181 || RPP25 | 3.34 | ribonuclease P/MRP 25kDa subunit | 1.144822637 |
3.34.181 || RSBN1 | 3.34 | round spermatid basic protein 1 | 0.275367056 | 3.34.181 || RSF1 | 3.34 |
remodeling and spacing factor 1 | 0.513250167 | 3.34.181 || RUFY2 | 3.34 | RUN and FYVE
domain containing 2 | 1.033520713 | 3.34.181 || RUFY3 | 3.34 | RUN and FYVE domain containing
3 | 1.145867001 | 3.34.181 || SCN9A | 3.34 | sodium voltage-gated channel alpha subunit 9 |
0.414266817 | 3.34.183 || SCYL3 | 3.34 | SCY1 like pseudokinase 3 | 1.898146295 | 3.34.181 ||
SDCBP2-AS1 | 3.34 | SDCBP2 antisense RNA 1 | 1.762024139 | 3.34.183 || SEL1L3 | 3.34 | SELIL
family member 3 | 2.154903827 | 3.34.181 || SELT | 3.34 | selenoprotein T | 0.426260752 | 3.34.181
|| SESN3 | 3.34 | sestrin 3 | 0.597934441 | 3.34.181 || SETD6 | 3.34 | SET domain containing 6 |
1.813748347 | 3.34.181 || SHPRH | 3.34 | SNF2 histone linker PHD RING helicase | 0.23418674 |
3.34.181 || SLC2A13 | 3.34 | solute carrier family 2 member 13 | 0.533238603 | 3.34.181 || SMAD5 |
3.34 | SMAD family member 5 | 0.525501557 | 3.34.181 || SNAPC3 | 3.34 | small nuclear RNA
activating complex polypeptide 3 | 0.319022756 | 3.34.183 || SNX24 | 3.34 | sorting nexin 24 |
0.447892012 | 3.34.183 || SON | 3.34 | SON DNA binding protein | 0.215952043 | 3.34.181 ||
SPDYE2 | 3.34 | speedy/RINGO cell cycle regulator family member E2 | 0.514703859 | 3.34.183 ||
SPINT2 | 3.34 | serine peptidase inhibitor, Kunitz type, 2 | 0.680207775 | 3.34.181 || SPRED3 | 3.34
| sprouty related, EVHI domain containing 3 | 0.414830031 | 3.34.182 || STK26 | 3.34 |
serine/threonine protein kinase 26 | 0.242031481 | 3.34.181 || STX17 | 3.34 | syntaxin 17 |
0.224097066 | 3.34.181 || TACC1 | 3.34 | transforming acidic coiled-coil containing protein 1 |
0.434972724 | 3.34.181 || TEF | 3.34 | thyrotrophic embryonic factor | 1.331753518 | 3.34.182 |
THUMPD1 | 3.34 | THUMP domain containing 1 | 0.260783091 | 3.34.181 || TLR5 | 3.34 | toll like
receptor 5 | 0.292363259 | 3.34.183 || TMEM181 | 3.34 | transmembrane protein 181 | 0.303971929 |
3.34.181 || TMUB2 | 3.34 | transmembrane and ubiquitin-like domain containing 2 | 0.32135527 |
3.34.183 || TNPO1 | 3.34 | transportin 1 | 0.408402369 | 3.34.181 || TNPO3 | 3.34 | transportin 3 |
0.218360117 | 3.34.181 || TNRC6B | 3.34 | trinucleotide repeat containing 6B | 0.453926376 |
3.34.181 || TOB2 | 3.34 | transducer of ERBB2, 2 | 0.449271682 | 3.34.182 || TOMM40 | 3.34 |
translocase of outer mitochondrial membrane 40 | 0.432087442 | 3.34.181 || TOR3A | 3.34 | torsin
family 3 member A | 0.431391398 | 3.34.181 || TPMT | 3.34 | thiopurine S-methyltransferase |
0.3476196 | 3.34.181 || TRABD | 3.34 | TraB domain containing | 0.490335244 | 3.34.181 ||
TRIM23 | 3.34 | tripartite motif containing 23 | 0.669486913 | 3.34.181 || TRIO | 3.34 | trio Rho
guanine nucleotide exchange factor | 0.432140335 | 3.34.183 || TSPAN31 | 3.34 | tetraspanin 31 |
0.283605503 | 3.34.181 || TTF1 | 3.34 | transcription termination factor, RNA polymerase I |
0.550104915 | 3.34.181 || TXNRD2 | 3.34 | thioredoxin reductase 2 | 0.526032852 | 3.34.181 ||
UBA5 | 3.34 | ubiquitin like modifier activating enzyme 5 | 0.216705036 | 3.34.182 || UBE2H | 3.34
| ubiquitin conjugating enzyme E2H | 0.256359853 | 3.34.182 || UBXN7 | 3.34 | UBX domain
protein 7 | 1.053709113 | 3.34.182 || VCPKMT | 3.34 | valosin containing protein lysine
methyltransferase | 0.226876516 | 3.34.182 || VOPP1 | 3.34 | vesicular, overexpressed in cancer,
prosurvival protein 1 | 0.310501971 | 3.34.181 || WIPF2 | 3.34 | WAS/WASL interacting protein
family member 2 | 0.254998218 | 3.34.181 || WWC3 | 3.34 | WWC family member 3 | 0.294881978
| 3.34.182 || ZADH2 | 3.34 | zinc binding alcohol dehydrogenase domain containing 2 | 0.228537977
| 3.34.183 || ZBTB21 | 3.34 | zinc finger and BTB domain containing 21 | 0.870634529 | 3.34.182 ||
ZBTB41 | 3.34 | zinc finger and BTB domain containing 41 | 0.489728574 | 3.34.181 || ZMAT1 |
3.34 | zinc finger matrin-type 1 | 0.791358786 | 3.34.181 || ZNF397 | 3.34 | zinc finger protein 397 |
1.410398387 | 3.34.181 || ZNF420 | 3.34 | zinc finger protein 420 | 0.531606442 | 3.34.181 ||
ZNF493 | 3.34 | zinc finger protein 493 | 1.478163813 | 3.34.181 || ZNF548 | 3.34 | zinc finger
protein 548 | 0.473137177 | 3.34.181 || ZNF565 | 3.34 | zinc finger protein 565 | 0.404727635 |
3.34.181 || ZNF566 | 3.34 | zinc finger protein 566 | 0.55922715 | 3.34.181 || ZNF573 | 3.34 | zinc
finger protein 573 | 1.58382204 | 3.34.181 || ZNF814 | 3.34 | zinc finger protein 814 | 1.889444811 |
3.34.181 || ACOX1 | 3.35.184 | acyl-CoA oxidase 1, palmitoyl | 0.467937072 | 3.35.184 ||
ANKRD17 | 3.35.184 | ankyrin repeat domain 17 | 0.215145854 | 3.35.184 || ATMIN | 3.35.184 |
ATM interactor | 0.309745304 | 3.35.184 || BBS7 | 3.35.184 | Bardet-Biedl syndrome 7 |
1.007546576 | 3.35.184 || C11orf54 | 3.35.184 | chromosome 11 open reading frame 54 |
0.276428138 | 3.35.184 || CDK8 | 3.35.184 | cyclin-dependent kinase 8 | 0.321384373 | 3.35.184 ||
CENPBDIP1 | 3.35.184 | CENPB DNA-binding domains containing 1 pseudogene 1 | 0.637831024
| 3.35.184 || EPC2 | 3.35.184 | enhancer of polycomb homolog 2 | 0.261908455 | 3.35.184 ||
FAM73A | 3.35.184 | family with sequence similarity 73 member A | 0.293431031 | 3.35.184 ||
FGF14-AS2 | 3.35.184 | FGF14 antisense RNA 2 | 0.488365792 | 3.35.184 || FLVCR1 | 3.35.184 |
feline leukemia virus subgroup C cellular receptor 1 | 0.517406166 | 3.35.184 || GUCY1A2 |
3.35.184 | guanylate cyclase 1, soluble, alpha 2 | 4.978071272 | 3.35.184 || LRIG2 | 3.35.184 |
leucine-rich repeats and immunoglobulin-like domains 2 | 0.348012871 | 3.35.184 || NACC2 |
3.35.184 | NACC family member 2 | 0.452874329 | 3.35.184 || NEK4 | 3.35.184 | NIMA related
kinase 4 | 0.258849738 | 3.35.184 || NIPSNAP3A | 3.35.184 | nipsnap homolog 3A (C. elegans) |
0.595808532 | 3.35.184 || PAQR3 | 3.35.184 | progestin and adipoQ receptor family member III |
0.219424326 | 3.35.184 || RBM48 | 3.35.184 | RNA binding motif protein 48 | 0.434774992 |
3.35.184 || SLFN5 | 3.35.184 | schlafen family member 5 | 0.466848118 | 3.35.184 || TMEM55A |
3.35.184 | transmembrane protein 55A | 0.410382637 | 3.35.184 || TUBE1 | 3.35.184 | tubulin
epsilon 1 | 0.699948186 | 3.35.184 || WDR43 | 3.35.184 | WD repeat domain 43 | 0.314939307 |
3.35.184 || XPNPEP3 | 3.35.184 | X-prolyl aminopeptidase 3, mitochondrial | 0.385966784 |
3.35.184 || ZNF25 | 3.35.184 | zinc finger protein 25 | 0.288185512 | 3.35.184 || ZNF770 | 3.35.184 |
zinc finger protein 770 | 0.359009093 | 3.35.184 || ADRBK2 | 3.44 | adrenergic, beta, receptor
kinase 2 | 0.779712165 | - || B4GALT2 | 3.44 | UDP-Gal:betaGlcNAc beta 1,4-
galactosyltransferase, polypeptide 2 | 0.307548356 | - || BNIP3L | 3.44 | BCL2/adenovirus E1B
19kDa interacting protein 3-like | 0.274315627 | - || C9orf3 | 3.44 | chromosome 9 open reading
frame 3 | 0.238713228 | - || CNPY2 | 3.44 | canopy FGF signaling regulator 2 | 0.222055667 | - ||
DIRAS1 | 3.44 | DIRAS family GTP binding RAS like 1 | 0.22355553 | - || FAM107B | 3.44 | family
with sequence similarity 107 member B | 0.261914467 | - || GOLM1 | 3.44 | golgi membrane protein
1 | 1.866380879 | - || KDELR3 | 3.44 | KDEL endoplasmic reticulum protein retention receptor 3 |
0.613356719 | - || LTBP1 | 3.44 | latent transforming growth factor beta binding protein 1 |
0.341844814 | - || MAGED1 | 3.44 | MAGE family member D1 | 0.450803272 | - || MAP3K1 | 3.44 |
mitogen-activated protein kinase kinase kinase 1, E3 ubiquitin protein ligase | 0.220210865 | - ||
MLF1 | 3.44 | myeloid leukemia factor 1 | 0.818667797 | - || MRC1 | 3.44 | mannose receptor, C
type 1 | 0.27381441 | - || NDUFA4L2 | 3.44 | NADH dehydrogenase (ubiquinone) 1 alpha
subcomplex, 4-like 2 | 2.386762336 | - || NPTX2 | 3.44 | neuronal pentraxin II | 2.959906407 | - ||
NUCB2 | 3.44 | nucleobindin 2 | 0.320303426 | - || PDE3A | 3.44 | phosphodiesterase 3A |
1.109327326 | - || PRAF2 | 3.44 | PRAI domain family member 2 | 0.247056102 | - || PTS | 3.44 | 6-
pyruvoyltetrahydropterin synthase | 0.27585133 | - || PXYLP1 | 3.44 | 2-phosphoxylose phosphatase
1 | 0.561769434 | - || RABAC1 | 3.44 | Rab acceptor 1 (prenylated) | 0.224482181 | - || RCN3 | 3.44 |
reticulocalbin 3 | 0.545215855 | - || SHMT2 | 3.44 | serine hydroxymethyltransferase 2 |
0.258627156 | - || SMOC2 | 3.44 | SPARC related modular calcium binding 2 | 0.758082061 | - ||
SNAP29 | 3.44 | synaptosome associated protein 29kDa | 0.524855101 | - || TCTN3 | 3.44 | tectonic
family member 3 | 0.214553148 | - || TMED3 | 3.44 | transmembrane p24 trafficking protein 3 |
0.342471233 | - || TMEM170B | 3.44 | transmembrane protein 170B | 1.361576158 | - || VKORC1 |
3.44 | vitamin K epoxide reductase complex subunit 1 | 0.518118866 | - || AFAP1 | 3.45 | actin
filament associated protein 1 | 0.239106171 | - || AKRIB1 | 3.45 | aldo-keto reductase family 1,
member B1 (aldose reductase) | 0.338264927 | - || APOC1 | 3.45 | apolipoprotein C-I | 1.577894627
| - || ATP5S | 3.45 | ATP synthase, H+ transporting, mitochondrial Fo complex subunit s (factor B) |
0.277658806 | - || BAZIA | 3.45 | bromodomain adjacent to zinc finger domain 1A | 0.292369598 | -
|| BCL11A | 3.45 | B-cell CLL/lymphoma 11A | 0.87469962 | - || C21orf2 | 3.45 | chromosome 21
open reading frame 2 | 0.345826639 | - || CCDC102B | 3.45 | coiled-coil domain containing 102B |
2.804011819 | - || CCNE2 | 3.45 | cyclin E2 | 0.306377397 | - || CLPTM1 | 3.45 | cleft lip and palate
associated transmembrane protein 1 | 0.622622157 | - || CREB3L1 | 3.45 | cAMP responsive
element binding protein 3-like 1 | 0.486568446 | - || DPP7 | 3.45 | dipeptidyl peptidase 7 |
0.263313768 | - || GATA6 | 3.45 | GATA binding protein 6 | 0.620084904 | - || GBA2 | 3.45 |
glucosidase, beta (bile acid) 2 | 0.223333178 | - || GMPPA | 3.45 | GDP-mannose pyrophosphorylase
A | 0.663005049 | - || HM13 | 3.45 | histocompatibility (minor) 13 | 0.344611217 | - || KRIT1 | 3.45 |
KRITI, ankyrin repeat containing | 0.627055949 | - || LAYN | 3.45 | layilin | 0.277894939 | - ||
MTSSIL | 3.45 | metastasis suppressor 1-like | 0.310872987 | - || NAGLU | 3.45 | N-
acetylglucosaminidase, alpha | 0.257961073 | - || NIP7 | 3.45 | NIP7, nucleolar pre-rRNA processing
protein | 0.981361551 | - || PDIA6 | 3.45 | protein disulfide isomerase family A member 6 |
0.235972168 | - || PEG10 | 3.45 | paternally expressed 10 | 0.374344447 | - || PYHIN1 | 3.45 | pyrin
and HIN domain family member 1 | 0.680809494 | - || RAB27A | 3.45 | RAB27A, member RAS
oncogene family | 0.225251538 | - || SECISBP2 | 3.45 | SECIS binding protein 2 | 0.301027343 | - ||
STRBP | 3.45 | spermatid perinuclear RNA binding protein | 0.610263558 | - || TMED9 | 3.45 |
transmembrane p24 trafficking protein 9 | 0.21865046 | - || TNFRSF10A | 3.45 | tumor necrosis
factor receptor superfamily member 10a | 0.505708166 | - || TRAK2 | 3.45 | trafficking protein,
kinesin binding 2 | 0.423216901 | - || ZC3H12C | 3.45 | zinc finger CCCH-type containing 12C |
0.364815283 |

### Example 2: Molecular Endotypes of RA Patient Whole Blood Sample

A method was carried out to characterize the molecular landscape of patients with rheumatoid arthritis (RA) by analyzing gene expression profiles from whole blood samples.

Full transcriptomic RNA sequencing was carried out on whole blood samples from 97 patients with RA as compared to 114 healthy patients (control).

In brief, whole blood was collected in PAXgene Blood RNA tubes. After removal of ribosomal RNA and globin transcripts with the Ribo-Zero Globin Removal kit (Illumina), stranded libraries were prepared with the TruSeq Library prep kit (Illumina) and hybridized to a flow cell for sequencing with the Illumina HiSeq platform. Raw RNAseq output counts were log 2 normalized using the R DESeq2 package. The top 5,000 row variance (top5k rowVar) genes determined using standard deviation between samples were retained for further analysis.

The top 5,000 row variance genes were analyzed by a suite of gene expression technologies, including Multiscale Embedded Gene Co-expression Network Analysis (MEGENA) to generate gene coexpression modules which were functionally annotated and correlated to various demographic traits, clinical features, and laboratory assays.

In brief, the MEGENA R package was used to generate a gene coexpression network by inputting the top5k rowVar genes. MEGENA multi-scale clustering analysis (MCA) formed lineages of gene modules followed by identification of densely intraconnected hub genes using multi-scale hub analysis (MHA). Modules were assigned “lineage” names based on their multiscale pedigree from the root MEGENA module. The prcomp package was utilized to perform singular value decomposition and calculate MEGENA module eigengenes (MEs), equivalent to the first principal component calculated amongst the variance of a given MEGENA module. MEGENA MEs were correlated to the numerically encoded sample traits.

A heatmap was generated using ComplexHeatmap visualizing the top 40 sample trait correlations to the MEGENA modules that were significantly correlated to cohort (or cluster). Module gene symbols were used to programmatically query the STRING database and calculate the percentage of genes within a given module predicted to have known protein-protein interactions (PPI) ranging from 0 to 100%.)

A gene set variation analysis (GSVA) (GSVA (V1.25.0) R software package) was carried out as a non-parametric, unsupervised method for estimating the variation of pre-defined gene sets over all MEGENA module log 2 gene expression values. Input genes were employed only if the interquartile range (IQR) of their expression across the samples was greater than 0. Enrichment scores (GSVA scores) were calculated non-parametrically using a Kolmogorov Smirnoff (KS)-like random walk statistic. The enrichment scores(ES) were the largest positive and negative random walk deviations from zero, respectively, for a particular sample amongst the module gene set. The GSVA scores were used as input for unsupervised stable k-means clustering, and four different disease phenotypes or clusters were identified (i.e., cluster 0, cluster 1, cluster 2, cluster 3). GSVA was performed using the significant MEGENA modules as gene signatures.

The MEs of the significant MEGENA modules were correlated to mean gene expression of a given module per patient and visualized using Complex heatmap. As shown in FIG. 7, columns of patients were clustered using idealized k-means clustering on 97 RA samples and four different disease phenotypes or clusters were identified (i.e., cluster 0, cluster 1, cluster 2, cluster 3). Also as shown in FIG. 7, rows correspond to various immune cell type and process modules (e.g., 32 LUGENE modules (IFN to T reg), IL23 complex, JAK1, JAK2, TYK2, GC (glucocorticoid) in vitro, core GC in vitro, Hu GC, Northcott GC) used to identify the four RA patient subsets. The heatmap color intensity represents the enrichment of gene signature, with red indicating increased enrichment and blue decreased enrichment, and white representing the intermediate levels. The gender and cohort associated with each patient is visualized at the bottom of the heatmap and the bar plot represents age of each patient. For example, Cluster 1: the most abnormal, while Cluster 0: the least abnormal. Also, for example, Cluster 0 and Cluster 2: not on GC, while Cluster 1 and Cluster 3: yes on GC). In brief, the various immune cell type and process modules, including the 32 LUGENE modules as well as those for steroid response and JAK/TYK pathways (e.g., GC steroid gene markers, JAK1, JAK2, and TYK2) can be associated to subsets with enriched treatment targets. Overall, stable k-means clustering of gene coexpression modules effectively segregated RA patient subsets into four different disease phenotypes which may be associated to different treatment targets and/or responsive to different treatments. Table 2 details the genes within the LUGENE modules, as shown in FIG. 7.

TABLE_2
Genes within the LUGENE modules.

| Gene | Category | Gene | Category | Gene | Category |
|---|---|---|---|---|---|
| CD160 | Anergic_Activated_T_cells | DNMT3B | JAK_module | IGLVI-70 | Plasma_Cells |
| CD244 | Anergic_Activated_T_cells | GTF2I | JAK_module | MZB1 | Plasma_Cells |
| CTLA4 | Anergic_Activated_T_cells | GHR | JAK_module | PRDM1 | Plasma_Cells |
| HAVCR2 | Anergic_Activated_T_cells | CTLA4 | JAK_module | SDC1 | Plasma_Cells |
| ICOS | Anergic_Activated_T_cells | PTK2B | JAK_module | THEMIS2 | Plasma_Cells |
| KLRG1 | Anergic_Activated_T_cells | SLC9A1 | JAK_module | TNFRSF17 | Plasma_Cells |
| LAG3 | Anergic_Activated_T_cells | ROCK2 | JAK_module | CEACAM1 | SNOR_Low_Up |
| PDCD1 | Anergic_Activated_T_cells | ZFP36L1 | JAK_module | FCGR1A | SNOR_Low_Up |
| IL1RN | Anti_inflammation | IL6ST | JAK_module | LGALS1 | SNOR_Low_Up |
| SOCS3 | Anti_inflammation | RACK1 | JAK_module | SNORD24 | SNOR_Low_Up |
| TNFAIP3 | Anti_inflammation | PPP2R1A | JAK_module | SNORD44 | SNOR_Low_Up |
| BANK1 | B_cells | DNMT3A | JAK_module | SNORD47 | SNOR_Low_Up |
| BLK | B_cells | PAK1 | JAK_module | SNORD80 | SNOR_Low_Up |
| BLNK | B_cells | JAK1 | JAK_module | CCR3 | T_Cells |
| CD19 | B_cells | PIK3R3 | JAK_module | CD226 | T_Cells |
| CD22 | B_cells | JAK2 | JAK_module | CD247 | T_Cells |
| CD79A | B_cells | MAP3K7 | JAK_module | CD28 | T_Cells |
| CD79B | B_cells | TRAF6 | JAK_module | CD3D | T_Cells |
| DAPP1 | B_cells | SHC1 | JAK_module | CD3E | T_Cells |
| FCRL1 | B_cells | PLCG1 | JAK_module | CD3G | T_Cells |
| FCRL2 | B_cells | PTPN11 | JAK_module | CD4 | T_Cells |
| FCRL3 | B_cells | IRS2 | JAK_module | CD5 | T_Cells |
| FCRLA | B_cells | STAT5A | JAK_module | CD8A | T_Cells |
| GON4L | B_cells | STAT5B | JAK_module | CD8B | T_Cells |
| GPR183 | B_cells | RAF1 | JAK_module | ETS1 | T_Cells |
| IGHD | B_cells | FYN | JAK_module | GATA3 | T_Cells |
| IGHM | B_cells | CXCR4 | JAK_module | GRAP2 | T_Cells |
| KLHL6 | B_cells | PIK3R2 | JAK_module | LEF1 | T_Cells |
| MS4A1 | B_cells | IRS1 | JAK_module | SH2D1A | T_Cells |
| PAX5 | B_cells | STAT1 | JAK_module | TRAC | T_Cells |
| PLCL2 | B_cells | PTK2 | JAK_module | TRBC1 | T_Cells |
| SH3BP5 | B_cells | MMP2 | JAK_module | TRDC | T_Cells |
| VPREB1 | B_cells | MAPK3 | JAK_module | FOXP3 | T_reg |
| ZNF318 | B_cells | MAPK1 | JAK_module | IKZF2 | T_reg |
| ASPM | Cell_Cycle | EGFR | JAK_module | TRAV10 | TCRA |
| AURKA | Cell_Cycle | STAT3 | JAK_module | TRAV1-1 | TCRA |
| AURKB | Cell_Cycle | PIK3R1 | JAK_module | TRAV1-2 | TCRA |
| BRCA1 | Cell_Cycle | CALM1 | JAK_module | TRAV12-1 | TCRA |
| CCNB1 | Cell_Cycle | CALM2 | JAK_module | TRAV12-2 | TCRA |
| CCNB2 | Cell_Cycle | CALM3 | JAK_module | TRAV12-3 | TCRA |
| CCNE1 | Cell_Cycle | AZU1 | LDG | TRAV13-1 | TCRA |
| CDC20 | Cell_Cycle | CAMP | LDG | TRAV13-2 | TCRA |
| CENPM | Cell_Cycle | CEACAM6 | LDG | TRAV14DV4 | TCRA |
| CEP55 | Cell_Cycle | CEACAM8 | LDG | TRAV16 | TCRA |
| E2F3 | Cell_Cycle | CTSG | LDG | TRAV17 | TCRA |
| GINS2 | Cell_Cycle | DEFA4 | LDG | TRAV18 | TCRA |
| MCM10 | Cell_Cycle | ELANE | LDG | TRAV19 | TCRA |
| MCM2 | Cell_Cycle | LCN2 | LDG | TRAV2 | TCRA |
| MKI67 | Cell_Cycle | LTF | LDG | TRAV20 | TCRA |
| NCAPG | Cell_Cycle | MPO | LDG | TRAV21 | TCRA |
| NDC80 | Cell_Cycle | OLFM4 | LDG | TRAV22 | TCRA |
| PTTG1 | Cell_Cycle | RNASE3 | LDG | TRAV23DV6 | TCRA |
| TYMS | Cell_Cycle | HLA-DMA | MHC_II | TRAV24 | TCRA |
| ZBTB16 | core_GC_invitro | HLA-DMB | MHC_II | TRAV25 | TCRA |
| FKBP5 | core_GC_invitro | HLA-DPA1 | MHC_II | TRAV26-1 | TCRA |
| TSC22D3 | core_GC_invitro | HLA-DPB1 | MHC_II | TRAV26-2 | TCRA |
| CLEC10A | Dendritic | HLA-DPB2 | MHC_II | TRAV27 | TCRA |
| CLEC12A | Dendritic | HLA-DQA1 | MHC_II | TRAV29DV5 | TCRA |
| CLEC9A | Dendritic | HLA-DQA2 | MHC_II | TRAV3 | TCRA |
| CSF1R | Dendritic | HLA-DQB1 | MHC_II | TRAV30 | TCRA |
| IGIP | Dendritic | HLA-DQB2 | MHC_II | TRAV34 | TCRA |
| LILRA4 | Dendritic | HLA-DRA | MHC_II | TRAV35 | TCRA |
| LY75 | Dendritic | HLA-DRB1 | MHC_II | TRAV36DV7 | TCRA |
| XCR1 | Dendritic | HLA-DRB3 | MHC_II | TRAV38-1 | TCRA |
| ZBTB16 | GC_module_invitro | HLA-DRB4 | MHC_II | TRAV38- | TCRA |
| | | | | 2DV8 | |
| FKBP5 | GC_module_invitro | HLA-DRB5 | MHC_II | TRAV39 | TCRA |
| TSC22D3 | GC_module_invitro | HLA-DRB6 | MHC_II | TRAV4 | TCRA |
| IL1R2 | GC_module_invitro | ACE | Monocyte | TRAV40 | TCRA |
| CD163 | GC_module_invitro | ADAM8 | Monocyte | TRAV41 | TCRA |
| TCN2 | GC_module_invitro | ADAMDEC1 | Monocyte | TRAV5 | TCRA |
| ADORA3 | GC_module_invitro | ADGRE1 | Monocyte | TRAV7 | TCRA |
| SAP30 | GC_module_invitro | ADGRE2 | Monocyte | TRAV8-1 | TCRA |
| SPTLC2 | GC_module_invitro | APOBEC3B | Monocyte | TRAV8-2 | TCRA |
| PCTP | GC_module_invitro | APOBEC3G | Monocyte | TRAV8-3 | TCRA |
| CPM | GC_module_invitro | APOBR | Monocyte | TRAV8-4 | TCRA |
| CLEC4E | GC_module_invitro | ART4 | Monocyte | TRAV8-6 | TCRA |
| LYVE1 | GC_module_invitro | C1QA | Monocyte | TRAV8-7 | TCRA |
| CPD | GC_module_invitro | C1QC | Monocyte | TRAV9-1 | TCRA |
| THBSI | GC_module_invitro | C2 | Monocyte | TRAV9-2 | TCRA |
| GRB10 | GC_module_invitro | C4A | Monocyte | TRAJ10 | TCRAJ |
| TRG-AS1 | gd_T_cells | C4B | Monocyte | TRAJ11 | TCRAJ |
| BLK | gd_T_cells | C4BPA | Monocyte | TRAJ12 | TCRAJ |
| TARP | gd_T_cells | C4BPB | Monocyte | TRAJ13 | TCRAJ |
| TRDC | gd_T_cells | C5 | Monocyte | TRAJ14 | TCRAJ |
| CD3E | gd_T_cells | C6 | Monocyte | TRAJ15 | TCRAJ |
| CD3G | gd_T_cells | C8A | Monocyte | TRAJ16 | TCRAJ |
| CD177 | Granulocyte | C9 | Monocyte | TRAJ17 | TCRAJ |
| CLC | Granulocyte | CCL17 | Monocyte | TRAJ18 | TCRAJ |
| CTSS | Granulocyte | CCL18 | Monocyte | TRAJ19 | TCRAJ |
| CXCR2 | Granulocyte | CCL22 | Monocyte | TRAJ20 | TCRAJ |
| DEFA1 | Granulocyte | CCL28 | Monocyte | TRAJ21 | TCRAJ |
| FUT7 | Granulocyte | CCL7 | Monocyte | TRAJ22 | TCRAJ |
| LTB4R | Granulocyte | CCL8 | Monocyte | TRAJ23 | TCRAJ |
| MMP25 | Granulocyte | CD14 | Monocyte | TRAJ24 | TCRAJ |
| OSM | Granulocyte | CD163 | Monocyte | TRAJ25 | TCRAJ |
| RETN | Granulocyte | CD209 | Monocyte | TRAJ26 | TCRAJ |
| FKBP5 | Hu_GC_Gene_Sig | CD300C | Monocyte | TRAJ27 | TCRAJ |
| ECHDC3 | Hu_GC_Gene_Sig | CD300E | Monocyte | TRAJ28 | TCRAJ |
| IL1R2 | Hu_GC_Gene_Sig | CD5L | Monocyte | TRAJ29 | TCRAJ |
| ZBTB16 | Hu_GC_Gene_Sig | CD80 | Monocyte | TRAJ3 | TCRAJ |
| IRS2 | Hu_GC_Gene_Sig | CFB | Monocyte | TRAJ30 | TCRAJ |
| IRAK3 | Hu_GC_Gene_Sig | CFD | Monocyte | TRAJ31 | TCRAJ |
| ACSL1 | Hu_GC_Gene_Sig | CFP | Monocyte | TRAJ32 | TCRAJ |
| DUSP1 | Hu_GC_Gene_Sig | CHI3L1 | Monocyte | TRAJ33 | TCRAJ |
| EIF2AK2 | IFN | CHIT1 | Monocyte | TRAJ34 | TCRAJ |
| GBP1 | IFN | CLEC12A | Monocyte | TRAJ35 | TCRAJ |
| GBP2 | IFN | CLEC12B | Monocyte | TRAJ36 | TCRAJ |
| GBP4 | IFN | CLEC4D | Monocyte | TRAJ37 | TCRAJ |
| HERC5 | IFN | CLEC4E | Monocyte | TRAJ38 | TCRAJ |
| HERC6 | IFN | CLEC5A | Monocyte | TRAJ39 | TCRAJ |
| IFI27 | IFN | CLEC7A | Monocyte | TRAJ4 | TCRAJ |
| IFI30 | IFN | CLEC9A | Monocyte | TRAJ40 | TCRAJ |
| IFI35 | IFN | CSF1R | Monocyte | TRAJ41 | TCRAJ |
| IFI44 | IFN | CXCL1 | Monocyte | TRAJ42 | TCRAJ |
| IFI44L | IFN | CXCL10 | Monocyte | TRAJ43 | TCRAJ |
| IFI6 | IFN | CXCL11 | Monocyte | TRAJ44 | TCRAJ |
| IFIT1 | IFN | CXCL13 | Monocyte | TRAJ45 | TCRAJ |
| IFIT2 | IFN | CXCL8 | Monocyte | TRAJ46 | TCRAJ |
| IFIT3 | IFN | CXCL9 | Monocyte | TRAJ47 | TCRAJ |
| IFIT5 | IFN | CYBB | Monocyte | TRAJ48 | TCRAJ |
| IFITM1 | IFN | F12 | Monocyte | TRAJ49 | TCRAJ |
| IFITM2 | IFN | FCGR3B | Monocyte | TRAJ5 | TCRAJ |
| IFITM3 | IFN | FFAR2 | Monocyte | TRAJ50 | TCRAJ |
| ISG15 | IFN | HMMR | Monocyte | TRAJ52 | TCRAJ |
| ISG20 | IFN | HVCN1 | Monocyte | TRAJ53 | TCRAJ |
| MX1 | IFN | IGSF6 | Monocyte | TRAJ54 | TCRAJ |
| MX2 | IFN | IL10RA | Monocyte | TRAJ56 | TCRAJ |
| OAS1 | IFN | IL12A | Monocyte | TRAJ57 | TCRAJ |
| OAS2 | IFN | IL12B | Monocyte | TRAJ58 | TCRAJ |
| OAS3 | IFN | IL1RAP | Monocyte | TRAJ59 | TCRAJ |
| OASL | IFN | IL20 | Monocyte | TRAJ6 | TCRAJ |
| RSAD2 | IFN | IL23A | Monocyte | TRAJ61 | TCRAJ |
| SAMD9 | IFN | IL27 | Monocyte | TRAJ7 | TCRAJ |
| SAMD9L | IFN | IL31RA | Monocyte | TRAJ8 | TCRAJ |
| SP100 | IFN | LAMP3 | Monocyte | TRAJ9 | TCRAJ |
| SP110 | IFN | LGALS12 | Monocyte | TRBJ2-1 | TCRB |
| IGHA1 | IG_Chains | LGALS4 | Monocyte | TRBJ2-2 | TCRB |
| IGHA2 | IG_Chains | LGALS9 | Monocyte | TRBJ2-2P | TCRB |
| IGHD2-15 | IG_Chains | LGALS9B | Monocyte | TRBJ2-3 | TCRB |
| IGHD2-2 | IG_Chains | LGALS9C | Monocyte | TRBJ2-4 | TCRB |
| IGHD2-21 | IG_Chains | LILRA2 | Monocyte | TRBJ2-5 | TCRB |
| IGHD3-10 | IG_Chains | LILRA5 | Monocyte | TRBJ2-6 | TCRB |
| IGHD3-16 | IG_Chains | LILRA6 | Monocyte | TRBJ2-7 | TCRB |
| IGHD3-3 | IG_Chains | LILRB5 | Monocyte | TRBV1 | TCRB |
| IGHD3-9 | IG_Chains | LMNB1 | Monocyte | TRBV10-1 | TCRB |
| IGHG1 | IG_Chains | LY86 | Monocyte | TRBV10-2 | TCRB |
| IGHG2 | IG_Chains | LYZ | Monocyte | TRBV11-1 | TCRB |
| IGHG3 | IG_Chains | MARCO | Monocyte | TRBV11-2 | TCRB |
| IGHG4 | IG_Chains | MEGF10 | Monocyte | TRBV19 | TCRB |
| IGHJ1 | IG_Chains | MERTK | Monocyte | TRBV2 | TCRB |
| IGHJ2 | IG_Chains | MFGE8 | Monocyte | TRBV20-1 | TCRB |
| IGHJ3 | IG_Chains | MPEG1 | Monocyte | TRBV21-1 | TCRB |
| IGHJ4 | IG_Chains | MRC1 | Monocyte | TRBV23-1 | TCRB |
| IGHJ5 | IG_Chains | MS4A4A | Monocyte | TRBV24-1 | TCRB |
| IGHJ6 | IG_Chains | MSR1 | Monocyte | TRBV25-1 | TCRB |
| IGHV1-18 | IG_Chains | NTSR1 | Monocyte | TRBV27 | TCRB |
| IGHV1-2 | IG_Chains | OCM | Monocyte | TRBV28 | TCRB |
| IGHV1-24 | IG_Chains | OLR1 | Monocyte | TRBV3-1 | TCRB |
| IGHV1-3 | IG_Chains | OSCAR | Monocyte | TRBV4-1 | TCRB |
| IGHV1-45 | IG_Chains | OTOF | Monocyte | TRBV4-2 | TCRB |
| IGHV1-46 | IG_Chains | PDCD1LG2 | Monocyte | TRBV5-1 | TCRB |
| IGHV1-58 | IG_Chains | PILRA | Monocyte | TRBV5-3 | TCRB |
| IGHV1-69-2 | IG_Chains | PLA2G2D | Monocyte | TRBV5-4 | TCRB |
| IGHV2-26 | IG_Chains | PLA2G5 | Monocyte | TRBV5-5 | TCRB |
| IGHV2-5 | IG_Chains | PYHIN1 | Monocyte | TRBV5-6 | TCRB |
| IGHV2-70 | IG_Chains | S100A8 | Monocyte | TRBV5-7 | TCRB |
| IGHV3-13 | IG_Chains | S100A9 | Monocyte | TRBV6-1 | TCRB |
| IGHV3-15 | IG_Chains | S1PR5 | Monocyte | TRBV6-4 | TCRB |
| IGHV3-20 | IG_Chains | SCARB1 | Monocyte | TRBV6-5 | TCRB |
| IGHV3-21 | IG_Chains | SCARF2 | Monocyte | TRBV6-6 | TCRB |
| IGHV3-23 | IG_Chains | SECTM1 | Monocyte | TRBV6-7 | TCRB |
| IGHV3-33 | IG_Chains | SEMA4A | Monocyte | TRBV6-8 | TCRB |
| IGHV3-43 | IG_Chains | SERPINB9 | Monocyte | TRBV7-1 | TCRB |
| IGHV3-48 | IG_Chains | SERPING1 | Monocyte | TRBV7-3 | TCRB |
| IGHV3-49 | IG_Chains | SIGLEC1 | Monocyte | TRBV7-4 | TCRB |
| IGHV3-53 | IG_Chains | SIGLEC14 | Monocyte | TRBV7-5 | TCRB |
| IGHV3-62 | IG_Chains | SIGLEC5 | Monocyte | TRBV7-6 | TCRB |
| IGHV3-64 | IG_Chains | SIGLEC7 | Monocyte | TRBV7-7 | TCRB |
| IGHV3-7 | IG_Chains | SLC11A1 | Monocyte | TRBV9 | TCRB |
| IGHV3-72 | IG_Chains | SLITRK4 | Monocyte | TRDC | TCRD |
| IGHV3-73 | IG_Chains | SMPDL3B | Monocyte | TRDJ1 | TCRD |
| IGHV3-74 | IG_Chains | SPIC | Monocyte | TRDJ2 | TCRD |
| IGHV4-28 | IG_Chains | STAB2 | Monocyte | TRDJ3 | TCRD |
| IGHV4-30-2 | IG_Chains | STAP2 | Monocyte | TRDJ4 | TCRD |
| IGHV4-34 | IG_Chains | TEK | Monocyte | TRDV1 | TCRD |
| IGHV4-39 | IG_Chains | TGM2 | Monocyte | TRDV2 | TCRD |
| IGHV4-59 | IG_Chains | TIMD4 | Monocyte | TRDV3 | TCRD |
| IGHV4-61 | IG_Chains | TLR2 | Monocyte | ACLY | TNF |
| IGHV5-51 | IG_Chains | TLR8 | Monocyte | ACSL1 | TNF |
| IGHV6-1 | IG_Chains | TNF | Monocyte | ADGRE2 | TNF |
| IGHV7-81 | IG_Chains | TNFAIP8L2 | Monocyte | AK3 | TNF |
| IGKC | IG_Chains | TNFRSF1B | Monocyte | AKAP10 | TNF |
| IGKJ1 | IG_Chains | TNIP3 | Monocyte | AMPD3 | TNF |
| IGKJ2 | IG_Chains | TULP1 | Monocyte | APOL3 | TNF |
| IGKJ3 | IG_Chains | UBD | Monocyte | ARID3A | TNF |
| IGKJ4 | IG_Chains | VENTX | Monocyte | ARSE | TNF |
| IGKJ5 | IG_Chains | VSTM1 | Monocyte | ASAP1 | TNF |
| IGKV1-16 | IG_Chains | ABHD3 | Neutrophil | B4GALT5 | TNF |
| IGKV1-17 | IG_Chains | ADAM8 | Neutrophil | BCL2A1 | TNF |
| IGKV1-27 | IG_Chains | CD177 | Neutrophil | BHLHE41 | TNF |
| IGKV1-5 | IG_Chains | CD83 | Neutrophil | BHMT | TNF |
| IGKV1-6 | IG_Chains | CLEC6A | Neutrophil | BIRC3 | TNF |
| IGKV1-9 | IG_Chains | LY6E | Neutrophil | BRCA1 | TNF |
| IGKV1D-16 | IG_Chains | S100A6 | Neutrophil | CALD1 | TNF |
| IGKV1D-17 | IG_Chains | KLRF1 | NK | CASP1 | TNF |
| IGKV1D-43 | IG_Chains | NCAM1 | NK | CASP10 | TNF |
| IGKV1D-8 | IG_Chains | NCR1 | NK | CCL15 | TNF |
| IGKV2-24 | IG_Chains | NCR3 | NK | CCL20 | TNF |
| IGKV2D-26 | IG_Chains | SH2D1B | NK | CCL23 | TNF |
| IGKV2D-29 | IG_Chains | IL1R2 | Northcott_GC_Gene_Sig | CCL3L1 | TNF |
| IGKV2D-30 | IG_Chains | CD163 | Northcott_GC_Gene_Sig | CD37 | TNF |
| IGKV3-20 | IG_Chains | ALOX15B | Northcott_GC_Gene_Sig | CD38 | TNF |
| IGKV3D-20 | IG_Chains | VSIG4 | Northcott_GC_Gene_Sig | CD83 | TNF |
| IGKV3D-7 | IG_Chains | AMPH | Northcott_GC_Gene_Sig | CDKN3 | TNF |
| IGKV4-1 | IG_Chains | FLT3 | Northcott_GC_Gene_Sig | CKB | TNF |
| IGKV5-2 | IG_Chains | ATP5A1 | Oxidative_Phosphorylation | CR2 | TNF |
| IGLC2 | IG_Chains | ATP5B | Oxidative_Phosphorylation | CTNND2 | TNF |
| IGLC7 | IG_Chains | ATP5D | Oxidative_Phosphorylation | CXCL1 | INF |
| IGLJ6 | IG_Chains | ATP5E | Oxidative_Phosphorylation | CXCL2 | TNF |
| IGLV10-54 | IG_Chains | ATP5F1 | Oxidative_Phosphorylation | CXCL3 | TNF |
| IGLV1-36 | IG_Chains | ATP5G1 | Oxidative_Phosphorylation | CXCL8 | TNF |
| IGLV1-40 | IG_Chains | ATP5G2 | Oxidative_Phosphorylation | CYP27B1 | TNF |
| IGLV1-47 | IG_Chains | ATP5G3 | Oxidative_Phosphorylation | DAB2 | TNF |
| IGLV2-11 | IG_Chains | ATP5H | Oxidative_Phosphorylation | EBI3 | TNF |
| IGLV2-18 | IG_Chains | ATP5I | Oxidative_Phosphorylation | EGR1 | TNF |
| IGLV2-23 | IG_Chains | ATP5J | Oxidative_Phosphorylation | EGR2 | TNF |
| IGLV2-33 | IG_Chains | ATP5J2 | Oxidative_Phosphorylation | EPB41 | TNF |
| IGLV2-8 | IG_Chains | ATP5L | Oxidative_Phosphorylation | EREG | TNF |
| IGLV3-1 | IG_Chains | ATP5O | Oxidative_Phosphorylation | ETAA1 | TNF |
| IGLV3-10 | IG_Chains | ATP5S | Oxidative_Phosphorylation | F3 | TNF |
| IGLV3-12 | IG_Chains | BCS1L | Oxidative_Phosphorylation | FABP1 | TNF |
| IGLV3-16 | IG_Chains | CEP89 | Oxidative_Phosphorylation | FBXL2 | TNF |
| IGLV3-19 | IG_Chains | COA1 | Oxidative_Phosphorylation | FCER2 | TNF |
| IGLV3-21 | IG_Chains | COA3 | Oxidative_Phosphorylation | FCGR2A | TNF |
| IGLV3-25 | IG_Chains | COA4 | Oxidative_Phosphorylation | FLJ11129 | TNF |
| IGLV3-27 | IG_Chains | COA5 | Oxidative_Phosphorylation | FLNA | TNF |
| IGLV3-32 | IG_Chains | COA6 | Oxidative_Phosphorylation | G0S2 | TNF |
| IGLV4-3 | IG_Chains | COA7 | Oxidative_Phosphorylation | GBP1 | TNF |
| IGLV4-60 | IG_Chains | COX10 | Oxidative_Phosphorylation | GCH1 | TNF |
| IGLV4-69 | IG_Chains | COX10-AS1 | Oxidative_Phosphorylation | GJB2 | TNF |
| IGLV5-37 | IG_Chains | COX11 | Oxidative_Phosphorylation | GLS | TNF |
| IGLV5-45 | IG_Chains | COX14 | Oxidative_Phosphorylation | GMIP | TNF |
| IGLV6-57 | IG_Chains | COX15 | Oxidative_Phosphorylation | GP1BA | TNF |
| IGLV7-43 | IG_Chains | COX16 | Oxidative_Phosphorylation | GRK3 | TNF |
| IGLV8-61 | IG_Chains | COX17 | Oxidative_Phosphorylation | HCAR3 | TNF |
| IGLV9-49 | IG_Chains | COX18 | Oxidative_Phosphorylation | HHEX | TNF |
| IGLVI-70 | IG_Chains | COX19 | Oxidative_Phosphorylation | HOMER2 | TNF |
| CHUK | ILPathway | COX20 | Oxidative_Phosphorylation | HP | TNF |
| IKBKB | ILPathway | COX411 | Oxidative_Phosphorylation | ICAM1 | TNF |
| NFKB1 | ILPathway | COX412 | Oxidative_Phosphorylation | IDO1 | TNF |
| MAP2K1 | ILPathway | COX5A | Oxidative_Phosphorylation | IFI44 | TNF |
| MAP2K4 | ILPathway | COX5B | Oxidative_Phosphorylation | IKBKG | TNF |
| IKBKG | ILPathway | COX6A1 | Oxidative_Phosphorylation | IL16 | TNF |
| IL1A | ILPathway | COX6A2 | Oxidative_Phosphorylation | IL18 | TNF |
| IL1R1 | ILPathway | COX6B1 | Oxidative_Phosphorylation | IL1A | TNF |
| IRAK1 | ILPathway | COX6B2 | Oxidative_Phosphorylation | IL1B | TNF |
| MAP3K3 | ILPathway | COX6C | Oxidative_Phosphorylation | IL1RN | TNF |
| MYD88 | ILPathway | COX7A1 | Oxidative_Phosphorylation | IL6 | TNF |
| MAP2K6 | ILPathway | COX7A2 | Oxidative_Phosphorylation | INHBA | TNF |
| MAP3K7 | ILPathway | COX7A2L | Oxidative_Phosphorylation | INSIG1 | TNF |
| TRAF6 | ILPathway | COX7B | Oxidative_Phosphorylation | ITGA6 | TNF |
| TAB1 | ILPathway | COX7B2 | Oxidative_Phosphorylation | KITLG | TNF |
| TAB2 | ILPathway | COX7C | Oxidative_Phosphorylation | KLF1 | TNF |
| IRAK4 | ILPathway | COX8A | Oxidative_Phosphorylation | KMO | TNF |
| TOLLIP | ILPathway | COX8C | Oxidative_Phosphorylation | LGALS3BP | TNF |
| MAP3K8 | ILPathway | CYC1 | Oxidative_Phosphorylation | MAP3K4 | TNF |
| IL1RAP | ILPathway | CYCS | Oxidative_Phosphorylation | MARCKS | TNF |
| IRAK2 | ILPathway | DNAJC15 | Oxidative_Phosphorylation | MGLL | TNF |
| MAPK8 | ILPathway | MT-ATP6 | Oxidative_Phosphorylation | MMP19 | TNF |
| RELA | ILPathway | MT-ATP8 | Oxidative_Phosphorylation | MN1 | TNF |
| TAB3 | ILPathway | MT-CO1 | Oxidative_Phosphorylation | MRPS15 | TNF |
| IL1B | ILPathway | MT-CO2 | Oxidative_Phosphorylation | MSC | TNF |
| UBE2N | ILPathway | MT-CO3 | Oxidative_Phosphorylation | MTF1 | TNF |
| SQSTM1 | ILPathway | MT-CYB | Oxidative_Phosphorylation | MX1 | TNF |
| IRAK3 | ILPathway | MT-ND1 | Oxidative_Phosphorylation | NAMPT | TNF |
| IL12B | ILComplex | MT-ND2 | Oxidative_Phosphorylation | NELL2 | TNF |
| IL12RB1 | ILComplex | MT-ND3 | Oxidative_Phosphorylation | NFKB1 | TNF |
| IL23A | ILComplex | MT-ND4 | Oxidative_Phosphorylation | NFKB2 | TNF |
| IL23R | ILComplex | MT-ND4L | Oxidative_Phosphorylation | NFKBIA | TNF |
| PSMB10 | Immunoproteasome | MT-ND5 | Oxidative_Phosphorylation | NFKBIZ | TNF |
| PSMB8 | Immunoproteasome | MT-ND6 | Oxidative_Phosphorylation | NKX3-2 | TNF |
| PSMB9 | Immunoproteasome | NDUFA1 | Oxidative_Phosphorylation | NR3C1 | TNF |
| AIM2 | Inflammasome | NDUFA10 | Oxidative_Phosphorylation | OAS3 | TNF |
| CASP1 | Inflammasome | NDUFA11 | Oxidative_Phosphorylation | PATJ | TNF |
| CASP5 | Inflammasome | NDUFA12 | Oxidative_Phosphorylation | PDE4DIP | TNF |
| CTSB | Inflammasome | NDUFA13 | Oxidative_Phosphorylation | PDPN | TNF |
| GSDMB | Inflammasome | NDUFA2 | Oxidative_Phosphorylation | PIAS4 | TNF |
| GSDMD | Inflammasome | NDUFA3 | Oxidative_Phosphorylation | PLAUR | TNF |
| NAIP | Inflammasome | NDUFA4 | Oxidative_Phosphorylation | PTGES | TNF |
| NEK7 | Inflammasome | NDUFA4L2 | Oxidative_Phosphorylation | PTGS2 | TNF |
| NLRC4 | Inflammasome | NDUFA5 | Oxidative_Phosphorylation | RELB | TNF |
| NLRP1 | Inflammasome | NDUFA6 | Oxidative_Phosphorylation | RPGR | TNF |
| NLRP3 | Inflammasome | NDUFA7 | Oxidative_Phosphorylation | RPS9 | TNF |
| NOD2 | Inflammasome | NDUFA8 | Oxidative_Phosphorylation | SDC4 | TNF |
| P2RX7 | Inflammasome | NDUFA9 | Oxidative_Phosphorylation | SERPIND1 | TNF |
| PANX1 | Inflammasome | NDUFAB1 | Oxidative_Phosphorylation | SFRP1 | TNF |
| PYCARD | Inflammasome | NDUFAF1 | Oxidative_Phosphorylation | SH3BP5 | TNF |
| RIPK1 | Inflammasome | NDUFAF2 | Oxidative_Phosphorylation | SLAMF1 | TNF |
| IL1A | Inflammatory_Cytokines | NDUFAF3 | Oxidative_Phosphorylation | SLC30A4 | TNF |
| CXCL10 | Inflammatory_Cytokines | NDUFAF4 | Oxidative_Phosphorylation | SOD2 | TNF |
| CXCL11 | Inflammatory_Cytokines | NDUFAF5 | Oxidative_Phosphorylation | SPI1 | TNF |
| CXCL9 | Inflammatory_Cytokines | NDUFAF6 | Oxidative_Phosphorylation | SSPN | TNF |
| TNFSF13B | Inflammatory_Cytokines | NDUFAF7 | Oxidative_Phosphorylation | STAT4 | TNF |
| CCL8 | Inflammatory_Cytokines | NDUFAF8 | Oxidative_Phosphorylation | TAF15 | TNF |
| TNF | Inflammatory_Cytokines | NDUFB1 | Oxidative_Phosphorylation | TAP2 | TNF |
| IL1B | Inflammatory_Cytokines | NDUFB10 | Oxidative_Phosphorylation | TBX3 | TNF |
| IL18 | Inflammatory_Cytokines | NDUFB11 | Oxidative_Phosphorylation | TFF1 | TNF |
| AOAH | Inhibitory_Macs | NDUFB2 | Oxidative_Phosphorylation | TNF | TNF |
| BACH1 | Inhibitory_Macs | NDUFB2- | Oxidative_Phosphorylation | TNFAIP2 | TNF |
| | | AS1 | | | |
| CD200R1 | Inhibitory_Macs | NDUFB3 | Oxidative_Phosphorylation | TNFAIP3 | TNF |
| CD300A | Inhibitory_Macs | NDUFB4 | Oxidative_Phosphorylation | TNFRSF11A | TNF |
| CD163 | Inhibitory_Macs | NDUFB5 | Oxidative_Phosphorylation | TRAF1 | TNF |
| CLEC7A | Inhibitory_Macs | NDUFB6 | Oxidative_Phosphorylation | TSC22D1 | TNF |
| SCARB1 | Inhibitory_Macs | NDUFB7 | Oxidative_Phosphorylation | TYROBP | TNF |
| MS4A4A | Inhibitory_Macs | NDUFB8 | Oxidative_Phosphorylation | UBE2C | TNF |
| IL10 | Inhibitory_Macs | NDUFB9 | Oxidative_Phosphorylation | VEGFA | TNF |
| TRIM6 | JAK_module | NDUFC1 | Oxidative_Phosphorylation | WT1 | TNF |
| FES | JAK_module | NDUFC2 | Oxidative_Phosphorylation | MPL | TYK_module |
| IL9R | JAK_module | NDUFS1 | Oxidative_Phosphorylation | PTAFR | TYK_module |
| IL10RB | JAK_module | NDUFS2 | Oxidative_Phosphorylation | SIVA1 | TYK_module |
| STAM | JAK_module | NDUFS3 | Oxidative_Phosphorylation | IL10RB | TYK_module |
| IL10RA | JAK_module | NDUFS4 | Oxidative_Phosphorylation | IL10RA | TYK_module |
| CSRP2 | JAK_module | NDUFS5 | Oxidative_Phosphorylation | STAT4 | TYK_module |
| INPP5D | JAK_module | NDUFS6 | Oxidative_Phosphorylation | TYK2 | TYK_module |
| IL4R | JAK_module | NDUFS7 | Oxidative_Phosphorylation | IFNAR1 | TYK_module |
| BTK | JAK_module | NDUFS8 | Oxidative_Phosphorylation | STAT6 | TYK_module |
| TYK2 | JAK_module | NDUFV1 | Oxidative_Phosphorylation | STAT2 | TYK_module |
| IFNGR1 | JAK_module | NDUFV2 | Oxidative_Phosphorylation | EIF2AK2 | TYK_module |
| STAT6 | JAK_module | NDUFV3 | Oxidative_Phosphorylation | CRKL | TYK_module |
| IL6R | JAK_module | NUBPL | Oxidative_Phosphorylation | MYLK | TYK_module |
| PLCG2 | JAK_module | OXA1L | Oxidative_Phosphorylation | IL6ST | TYK_module |
| STAT2 | JAK_module | RFESD | Oxidative_Phosphorylation | RACK1 | TYK_module |
| PRLR | JAK_module | SCO1 | Oxidative_Phosphorylation | CBL | TYK_module |
| EIF2AK2 | JAK_module | SCO2 | Oxidative_Phosphorylation | IRS2 | TYK_module |
| LCK | JAK_module | SLC25A4 | Oxidative_Phosphorylation | STAT5A | TYK_module |
| PTK2B | JAK_module | SURF1 | Oxidative_Phosphorylation | STAT5B | TYK_module |
| IL6ST | JAK_module | TACO1 | Oxidative_Phosphorylation | RAF1 | TYK_module |
| RACK1 | JAK_module | TIMMDC1 | Oxidative_Phosphorylation | IRS1 | TYK_module |
| PIK3R3 | JAK_module | TMEM126B | Oxidative_Phosphorylation | STAT1 | TYK_module |
| PTPN11 | JAK_module | TRAP1 | Oxidative_Phosphorylation | STAT3 | TYK_module |
| IRS2 | JAK_module | TTC19 | Oxidative_Phosphorylation | B4GALT3 | Unfolded_Protein |
| STAT5A | JAK_module | UQCC1 | Oxidative_Phosphorylation | CALR | Unfolded_Protein |
| STAT5B | JAK_module | UQCC2 | Oxidative_Phosphorylation | CALU | Unfolded_Protein |
| RAF1 | JAK_module | UQCC3 | Oxidative_Phosphorylation | CANX | Unfolded_Protein |
| FYN | JAK_module | UQCR10 | Oxidative_Phosphorylation | CDS2 | Unfolded_Protein |
| PIK3R2 | JAK_module | UQCR11 | Oxidative_Phosphorylation | CHST12 | Unfolded_Protein |
| IRS1 | JAK_module | UQCRB | Oxidative_Phosphorylation | CHST2 | Unfolded_Protein |
| SIRT1 | JAK_module | UQCRC1 | Oxidative_Phosphorylation | DERL1 | Unfolded_Protein |
| GRB2 | JAK_module | UQCRC2 | Oxidative_Phosphorylation | DERL2 | Unfolded_Protein |
| STAT1 | JAK_module | UQCRFS1 | Oxidative_Phosphorylation | DNAJC3 | Unfolded_Protein |
| STAT3 | JAK_module | UQCRH | Oxidative_Phosphorylation | EDEM2 | Unfolded_Protein |
| PIK3R1 | JAK_module | UQCRHL | Oxidative_Phosphorylation | EDEM3 | Unfolded_Protein |
| SH2B2 | JAK_module | UQCRQ | Oxidative_Phosphorylation | EMC9 | Unfolded_Protein |
| MPL | JAK_module | CLEC4C | pDC | ERAP1 | Unfolded_Protein |
| TUB | JAK_module | NRP1 | pDC | ERGIC2 | Unfolded_Protein |
| MST1R | JAK_module | IL3RA | pDC | ERO1L | Unfolded_Protein |
| IL5RA | JAK_module | C19orf10 | Plasma_Cells | EXT1 | Unfolded_Protein |
| STAM | JAK_module | IGH | Plasma_Cells | GALNT2 | Unfolded_Protein |
| STAM2 | JAK_module | IGHD | Plasma_Cells | GOLT1B | Unfolded_Protein |
| KANK1 | JAK_module | IGHG1 | Plasma_Cells | HERPUD1 | Unfolded_Protein |
| LEPR | JAK_module | IGHMBP2 | Plasma_Cells | HYOU1 | Unfolded_Protein |
| KCNN4 | JAK_module | IGHV2-5 | Plasma_Cells | IER3IP1 | Unfolded_Protein |
| SH2B1 | JAK_module | IGHV3-20 | Plasma_Cells | IMPAD1 | Unfolded_Protein |
| SIRPA | JAK_module | IGHV3-23 | Plasma_Cells | KDELC1 | Unfolded_Protein |
| ARHGEF1 | JAK_module | IGHV4-28 | Plasma_Cells | KDELR2 | Unfolded_Protein |
| EPOR | JAK_module | IGHV4-31 | Plasma_Cells | LMAN2 | Unfolded_Protein |
| KDM3A | JAK_module | IGHV4-34 | Plasma_Cells | LPGAT1 | Unfolded_Protein |
| CCR2 | JAK_module | IGK | Plasma_Cells | MAN1A1 | Unfolded_Protein |
| NFIC | JAK_module | IGKC | Plasma_Cells | MANEA | Unfolded_Protein |
| RAPGEF1 | JAK_module | IGL | Plasma_Cells | MANF | Unfolded_Protein |
| CSF2RB | JAK_module | IGLJ3 | Plasma_Cells | NUCB2 | Unfolded_Protein |
| STAT4 | JAK_module | IGLL1 | Plasma_Cells | PDIA4 | Unfolded_Protein |
| PAX5 | JAK_module | IGLV@ | Plasma_Cells | PDIA6 | Unfolded_Protein |
| BTK | JAK_module | IGLV1-40 | Plasma_Cells | PIGK | Unfolded_Protein |
| VAV1 | JAK_module | IGLV1-44 | Plasma_Cells | PPIB | Unfolded_Protein |
| CYBB | JAK_module | IGLV2-14 | Plasma_Cells | SEC24D | Unfolded_Protein |
| GAB2 | JAK_module | IGLV2-5 | Plasma_Cells | SEC61G | Unfolded_Protein |
| IFNGR1 | JAK_module | IGLV3-1 | Plasma_Cells | SPCS3 | Unfolded_Protein |
| PDK1 | JAK_module | IGLV3-19 | Plasma_Cells | SSR1 | Unfolded_Protein |
| STAT6 | JAK_module | IGLV3-25 | Plasma_Cells | SSR3 | Unfolded_Protein |
| PPP2R1B | JAK_module | IGLV4-3 | Plasma_Cells | TRAM1 | Unfolded_Protein |
| SH2B3 | JAK_module | IGLV4-60 | Plasma_Cells | TRAM2 | Unfolded_Protein |
| PLCG2 | JAK_module | IGLV5-45 | Plasma_Cells | UGGT1 | Unfolded_Protein |
| STAT2 | JAK_module | IGLV6-57 | Plasma_Cells | XBP1 | Unfolded_Protein |
| PRLR | JAK_module | | | | |

### Example 3: Molecular Endotypes of RA Patient Whole Blood Sample

A method was carried out to characterize the molecular landscape of patients with rheumatoid arthritis (RA) by analyzing gene expression profiles from whole blood samples. Whole blood samples were collected from 2 patient populations: (1) patients with rheumatoid arthritis (RA) that are incomplete responders (IR) to disease-modifying antirheumatic drug (DMARD) treatment (DMARD-IR), and (2) patients with rheumatoid arthritis (RA) that are incomplete responders (IR) to TNF Inhibitor treatment (TNF-IR).

Full transcriptomic RNA sequencing was carried out on whole blood samples collected from 2 patient populations as compared to healthy patients (control). The RNA sequencing assay involved 378 RA DMARD-IR patients, 115 RA TNF-IR patients, as compared to 20 healthy patients (control).

In brief, whole blood was collected in PAXgene Blood RNA tubes. RNA quality was determined on Agilent Bioanalyzer using the Agilent RNA 6000 Nano kit and quantified on a Nanodrop ND-1000 spectrophotometer. Total RNA was used for assessing transcriptome. Total RNA was converted to complementary RNA (cRNA) and hybridized to Affymetrix HU133plus2 chips that had 54,675 probe sets corresponding to the entire human genome. Hybridized chips were scanned using an Affymetrix Genechip Scanner 3000. Data from the microarray chips was normalized and analyzed using the MAS 5.0 algorithm, then imported into Array Assist software (Stratagene) and analyzed using Significance Analysis of Microarray (SAM) analysis. Mann-Whitney U test, Chi-square test, Pearson correlation and Spearman rank correlation analyses were used as and when required. Graphpad Prism (v9.1) was used for graphical representation of the results. R packages or Ingenuity Pathway Analysis were used to perform functional network analysis as explained below.

The Weighted Gene Co-expression Network Analysis (WGCNA) algorithm was used to construct co-expressed gene network modules that were assessed further for their functional significance. Raw microarray data files underwent background correction and GCRMA normalization resulting in log 2 intensity values compiled into an expression set object (e-set). The e-set was then restricted to the top 5000 probes with the highest variance.

A scale-free topology matrix (TOM) was calculated to encode the network strength between probes with a soft thresholding power of 30. TOM distances were used to cluster probes into WGCNA modules. Resulting co-expression networks were trimmed using dynamic tree cutting and the deepSplit function in R. Partitioning around medoids (PAM) was also utilized to assign outliers to the nearest cluster. The resulting network was formed with a minimum module size of 100, cut height of 1, and merge height of 0.2. Modules were given random color assignments and expression profiles summarized by a module eigengene (ME). Final membership of probes representing the same gene were decided based on strongest within-module correlation to the ME value. For each module, ME values were correlated by Pearson correlation to the clinical data including cohort (MMP-high group=1, MMP-low group=0), ESR, CRP, age, sex, swollen joints, disease duration, tender joints, and total affected joints. Significance was determined using an adjusted p-value≤0.2. MEGENA is a multi-scale co-expression gene clustering algorithm, which was used to create additional gene expression networks by applying it on the normalized and filtered gene modules from WGCNA. Multi-scale clustering structures were identified using planar filtered networks and resultant gene co-expression modules were also correlated to clinical metadata as described for WGCNA.

In brief, the MEGENA R package was used to generate a gene coexpression network by inputting the top5k rowVar genes. MEGENA multi-scale clustering analysis (MCA) formed lineages of gene modules followed by identification of densely intraconnected hub genes using multi-scale hub analysis (MHA). Modules were assigned “lineage” names based on their multiscale pedigree from the root MEGENA module. The prcomp package was utilized to perform singular value decomposition and calculate MEGENA module eigengenes (MEs), equivalent to the first principal component calculated amongst the variance of a given MEGENA module. MEGENA MEs were correlated to the numerically encoded sample traits.

A heatmap was generated using ComplexHeatmap visualizing the top 40 sample trait correlations to the MEGENA modules that were significantly correlated to cohort (or cluster.) Module gene symbols were used to programmatically query the STRING database and calculate the percentage of genes within a given module predicted to have known protein-protein interactions (PPI) ranging from 0 to 100%.

A gene set variation analysis (GSVA) (GSVA (V1.25.0) R software package) was carried out as a non-parametric, unsupervised method for estimating the variation of pre-defined gene sets over all MEGENA module log 2 gene expression values. Input genes were employed only if the interquartile range (IQR) of their expression across the samples was greater than 0. Enrichment scores (GSVA scores) were calculated non-parametrically using a Kolmogorov Smirnoff (KS)-like random walk statistic. The enrichment scores(ES) were the largest positive and negative random walk deviations from zero, respectively, for a particular sample amongst the module gene set. The GSVA scores were used as input for unsupervised stable k-means clustering, and five different disease phenotypes or clusters were identified (i.e., cluster 0, cluster 1, cluster 2, cluster 3, cluster 4). GSVA was performed using the significant MEGENA modules as gene signatures.

The MEs of the significant MEGENA modules were correlated to mean gene expression of a given module per patient and visualized using Complex heatmap. The heatmap color intensity represents the enrichment of gene signature, with red indicating increased enrichment and blue decreased enrichment, and white representing the intermediate levels. The cohort associated with each patient is visualized at the bottom of the heatmap. As shown in FIG. 8, columns of patients with RA that are DMARD-IR were clustered using idealized k-means clustering on patient samples and five different disease phenotypes or clusters were identified (i.e., cluster 0, cluster 1, cluster 2, cluster 3, cluster 4).

Also as shown in FIG. 8, rows correspond to various immune cell type and process modules (e.g., modules associated to inflammation, modules associated to cells, modules associated to JAK/TYK2, modules associated to GC steroids) used to identify the five patient subsets. As shown in FIG. 9, columns of patients with RA that are TNF-IR were clustered using idealized k-means clustering on patient samples and five different disease phenotypes or clusters were identified (i.e., cluster 0, cluster 1, cluster 2, cluster 3, cluster 4). Also as shown in FIG. 9, rows correspond to various immune cell type and process modules (e.g., modules associated to inflammation, modules associated to cells, modules associated to JAK/TYK2, modules associated to GC steroids) used to identify the five patient subsets. In brief, the modules, including those for steroid response and JAK/TYK pathways (e.g., GC steroid gene markers, JAK1, JAK2, and TYK2) can be associated to subsets with enriched treatment targets. Overall, stable k-means clustering of gene coexpression modules effectively segregated patient subsets into five different disease phenotypes which may be associated to different treatment targets and/or responsive to different treatments.

### Example 4: Molecular Endotypes of RA Patient Synovium Sample

A method was carried out to characterize the molecular landscape of patients with rheumatoid arthritis (RA) by analyzing gene expression profiles from synovium samples. Synovium samples were collected from a patient population comprising patients with rheumatoid arthritis (RA) that are incomplete responders (IR) to DMARD treatment (RA DMARD-IR patients) treated with TNFi.

Full transcriptomic RNA sequencing was carried out on synovium samples collected from a patient population. The RNA sequencing assay involved 46 RA DMARD-IR patients treated with TNFi.

In brief, synovium samples were collected. After removal of ribosomal RNA and globin transcripts with the Ribo-Zero Globin Removal kit (Illumina), stranded libraries are prepared with the TruSeq Library prep kit (Illumina) and hybridized to a flow cell for sequencing with the Illumina HiSeq platform. Raw RNAseq output counts are log 2 normalized using the R DESeq2 package. The top 5,000 row variance (top5k rowVar) genes determined using standard deviation between samples were retained for further analysis.

The top 5,000 row variance genes were analyzed by a suite of gene expression technologies, including Multiscale Embedded Gene Co-expression Network Analysis (MEGENA) to generate gene coexpression modules which were functionally annotated and correlated to various demographic traits, clinical features, and laboratory assays.

In brief, the MEGENA R package was used to generate a gene coexpression network by inputting the top5k rowVar genes. MEGENA multi-scale clustering analysis (MCA) formed lineages of gene modules followed by identification of densely intraconnected hub genes using multi-scale hub analysis (MHA). Modules were assigned “lineage” names based on their multiscale pedigree from the root MEGENA module. The prcomp package was utilized to perform singular value decomposition and calculate MEGENA module eigengenes (MEs), equivalent to the first principal component calculated amongst the variance of a given MEGENA module. MEGENA MEs were correlated to the numerically encoded sample traits.

A heatmap was generated using ComplexHeatmap visualizing the top 40 sample trait correlations to the MEGENA modules that were significantly correlated to cohort (or cluster.) Module gene symbols were used to programmatically query the STRING database and calculate the percentage of genes within a given module predicted to have known protein-protein interactions (PPI) ranging from 0 to 100%.

A gene set variation analysis (GSVA) (GSVA (V1.25.0) R software package) was carried out as a non-parametric, unsupervised method for estimating the variation of pre-defined gene sets over all MEGENA module log 2 gene expression values. Input genes were employed only if the interquartile range (IQR) of their expression across the samples was greater than 0. Enrichment scores (GSVA scores) were calculated non-parametrically using a Kolmogorov Smirnoff (KS)-like random walk statistic. The enrichment scores(ES) were the largest positive and negative random walk deviations from zero, respectively, for a particular sample amongst the module gene set. The GSVA scores were used as input for unsupervised stable k-means clustering, and five different disease phenotypes or clusters were identified (i.e., cluster 0, cluster 1, cluster 2, cluster 3, cluster 4). GSVA was performed using the significant MEGENA modules as gene signatures.

The MEs of the significant MEGENA modules were correlated to mean gene expression of a given module per patient and visualized using Complex heatmap. As shown in FIG. 10, columns of patients with RA that are TNF-IR were clustered using idealized k-means clustering on patient samples and three different disease phenotypes or clusters were identified (i.e., cluster 1, cluster 2, cluster 3). Also as shown in FIG. 10, rows correspond to various immune cell type and process modules (e.g., Endo modules: A, B, C, D, E, F, and G) used to identify the three patient subsets. In brief, the modules, can be associated to subsets with enriched treatment targets. Overall, stable k-means clustering of gene coexpression modules effectively segregated patient subsets into three different disease phenotypes which may be associated to different treatment targets and/or responsive to different treatments. Overall, FIG. 10 shows clinical response of RA DMARD-IR patients treated with TNFi. As shown in FIG. 10A, rows correspond to various EULAR response criteria (i.e., subject trait or patient trait), used to classify individual patients as non-responders, moderate responders, or high responders, depending on the extent of change and the level of disease activity reached. As shown in FIG. 10A, rows visualize in color (color choice arbitrarily selected to ease visualization) whether the EULAR response criteria (e.g., patient traits or subject traits: patient is female (pt.is.female), patient ancestry (pt.anc.), patient ancestry African (pt.anc.AA), patient ancestry Asian (pt.anc.AsA), patient ancestry Caribean (pt.anc.Carib), patient ancestry European (pt.anc.EA), patient treatment certolizumab pegol (CZP) (pt.treatment.CZP), patient treatment ETC (pt.treatment.ETC), patient non responder (patient.responder.non), patient moderate responder (patient.responder.mod), patient high responder (patient.responder.high), patient pauci-immune designation (pt.histo.pauci.immune), patient biopsy from wrist (pt.biop.wrist), patient biopsy from knee (pt.biop.knee), patient indication of RA erosion (pt.ra.erosion), patient pathology myeloid (pt.patho.myeloid), patient pathology lymphoid (pt.patho.lymphoid), patient pathology fibroid (pt.patho.fibroid), etc.) was met. Gradations of color intensity, from low color intensity to high color intensity, indicates degree to which criteria was met (e.g., continuous values: patient age (pt.age), patient inflammatory score (pt.score.inf), patient DAS28 score (pt.score.das28), patient change in DAS28 score after treatment (pt.score.das28.delta), patient HAQ score disease index (pt.score.haq.di), patient disease duration (pt.disease.duration), number of swollen joints (pt.count.joint.swollen), number of tender joints (pt.count.joint.tender), CRP C-reactive protein level (pt.CRP), patient erythrocyte sedimentation rate (ESR pt.ESR), patient rheumatoid factor (pt.RF), patient ACPA level (pt.ACPA), patient blood sample RNA concentration (pt.RNA.concentration), patient blood sample total RNA volume (pt.RNA.volume), patient blood sample total RNA yield (pt.RNA.yield), etc.). Also as shown in FIG. 10A, no color intensity shows DMARD-IR patients did not respond to TNFi treatment, low color intensity shows DMARD-IR patients were moderate responders to TNFi treatment, high color intensity shows DMARD-IR patients were high responders to TNFi treatment. Overall, the heatmap in FIG. 10A (see dark green at the bottom) shows high responders to TNFi identified in Cluster 3, as compared to low responders to TNFi identified in Cluster 1.

As shown in FIG. 10B, the heatmap color intensity represents the enrichment of gene signature, with bright red indicating increased enrichment and blue decreased enrichment, and white representing the intermediate levels. In brief, a high GSVA score (bright red) indicates a high response to TNFi therapy. Also as shown in both FIG. 10B and FIG. 10C, rows correspond to various immune cell type and process modules (e.g., Endo modules: A, B, C, D, E, F, and G) used to identify the patient subsets. The columns in FIG. 10C correspond to various clinical criteria. Overall, FIG. 10C details correlations between the various immune cell type and process modules (Endo modules) and the clinical criteria. As shown in FIG. 10C, rows visualize correlations in color, ranging from negative (blue, or 0) to positive (red, or 1), with white representing intermediate. Table 3 details the genes within the Endo modules.

TABLE_3
Genes within the Endo modules.

| Gene | Category | Gene | Category | Gene | Category |
|---|---|---|---|---|---|
| CD160 | Anergic/Activated.T.cells | C4BPB | Monocyte | IKZF2 | T.reg |
| CD244 | Anergic/Activated.T.cells | C5 | Monocyte | TRAV10 | TCRA |
| CTLA4 | Anergic/Activated.T.cells | C6 | Monocyte | TRAV1-1 | TCRA |
| HAVCR2 | Anergic/Activated.T.cells | C8A | Monocyte | TRAV1-2 | TCRA |
| ICOS | Anergic/Activated.T.cells | C9 | Monocyte | TRAV12-1 | TCRA |
| KLRG1 | Anergic/Activated.T.cells | CCL17 | Monocyte | TRAV12-2 | TCRA |
| LAG3 | Anergic/Activated.T.cells | CCL18 | Monocyte | TRAV12-3 | TCRA |
| PDCD1 | Anergic/Activated.T.cells | CCL22 | Monocyte | TRAV13-1 | TCRA |
| IL1RN | Anti.inflammation | CCL28 | Monocyte | TRAV13-2 | TCRA |
| SOCS3 | Anti.inflammation | CCL7 | Monocyte | TRAV14DV4 | TCRA |
| TNFAIP3 | Anti.inflammation | CCL8 | Monocyte | TRAV16 | TCRA |
| BANK1 | B.cells | CD14 | Monocyte | TRAV17 | TCRA |
| BLK | B.cells | CD163 | Monocyte | TRAV18 | TCRA |
| BLNK | B.cells | CD209 | Monocyte | TRAV19 | TCRA |
| CD19 | B.cells | CD300C | Monocyte | TRAV2 | TCRA |
| CD22 | B.cells | CD300E | Monocyte | TRAV20 | TCRA |
| CD79A | B.cells | CD5L | Monocyte | TRAV21 | TCRA |
| CD79B | B.cells | CD80 | Monocyte | TRAV22 | TCRA |
| DAPP1 | B.cells | CFB | Monocyte | TRAV23DV6 | TCRA |
| FCRL1 | B.cells | CFD | Monocyte | TRAV24 | TCRA |
| FCRL2 | B.cells | CFP | Monocyte | TRAV25 | TCRA |
| FCRL3 | B.cells | CHI3L1 | Monocyte | TRAV26-1 | TCRA |
| FCRLA | B.cells | CHIT1 | Monocyte | TRAV26-2 | TCRA |
| GON4L | B.cells | CLEC12A | Monocyte | TRAV27 | TCRA |
| GPR183 | B.cells | CLEC12B | Monocyte | TRAV29DV5 | TCRA |
| IGHD | B.cells | CLEC4D | Monocyte | TRAV3 | TCRA |
| IGHM | B.cells | CLEC4E | Monocyte | TRAV30 | TCRA |
| KLHL6 | B.cells | CLEC5A | Monocyte | TRAV34 | TCRA |
| MS4A1 | B.cells | CLEC7A | Monocyte | TRAV35 | TCRA |
| PAX5 | B.cells | CLEC9A | Monocyte | TRAV36DV7 | TCRA |
| PLCL2 | B.cells | CSFIR | Monocyte | TRAV38-1 | TCRA |
| SH3BP5 | B.cells | CXCL1 | Monocyte | TRAV38-2DV8 | TCRA |
| VPREB1 | B.cells | CXCL10 | Monocyte | TRAV39 | TCRA |
| ZNF318 | B.cells | CXCL11 | Monocyte | TRAV4 | TCRA |
| ASPM | Cell.Cycle | CXCL13 | Monocyte | TRAV40 | TCRA |
| AURKA | Cell.Cycle | CXCL8 | Monocyte | TRAV41 | TCRA |
| AURKB | Cell.Cycle | CXCL9 | Monocyte | TRAV5 | TCRA |
| BRCA1 | Cell.Cycle | CYBB | Monocyte | TRAV7 | TCRA |
| CCNB1 | Cell.Cycle | F12 | Monocyte | TRAV8-1 | TCRA |
| CCNB2 | Cell.Cycle | FCGR3B | Monocyte | TRAV8-2 | TCRA |
| CCNE1 | Cell.Cycle | FFAR2 | Monocyte | TRAV8-3 | TCRA |
| CDC20 | Cell.Cycle | HMMR | Monocyte | TRAV8-4 | TCRA |
| CENPM | Cell.Cycle | HVCN1 | Monocyte | TRAV8-6 | TCRA |
| CEP55 | Cell.Cycle | IGSF6 | Monocyte | TRAV8-7 | TCRA |
| E2F3 | Cell.Cycle | IL10RA | Monocyte | TRAV9-1 | TCRA |
| GINS2 | Cell.Cycle | IL12A | Monocyte | TRAV9-2 | TCRA |
| MCM10 | Cell.Cycle | IL12B | Monocyte | TRAJ10 | TCRAJ |
| MCM2 | Cell.Cycle | IL1RAP | Monocyte | TRAJ11 | TCRAJ |
| MKI67 | Cell.Cycle | IL20 | Monocyte | TRAJ12 | TCRAJ |
| NCAPG | Cell.Cycle | IL23A | Monocyte | TRAJ13 | TCRAJ |
| NDC80 | Cell.Cycle | IL27 | Monocyte | TRAJ14 | TCRAJ |
| PTTG1 | Cell.Cycle | IL31RA | Monocyte | TRAJ15 | TCRAJ |
| TYMS | Cell.Cycle | LAMP3 | Monocyte | TRAJ16 | TCRAJ |
| CLEC10A | Dendritic | LGALS12 | Monocyte | TRAJ17 | TCRAJ |
| CLEC12A | Dendritic | LGALS4 | Monocyte | TRAJ18 | TCRAJ |
| CLEC9A | Dendritic | LGALS9 | Monocyte | TRAJ19 | TCRAJ |
| CSF1R | Dendritic | LGALS9B | Monocyte | TRAJ20 | TCRAJ |
| IGIP | Dendritic | LGALS9C | Monocyte | TRAJ21 | TCRAJ |
| LILRA4 | Dendritic | LILRA2 | Monocyte | TRAJ22 | TCRAJ |
| LY75 | Dendritic | LILRA5 | Monocyte | TRAJ23 | TCRAJ |
| XCR1 | Dendritic | LILRA6 | Monocyte | TRAJ24 | TCRAJ |
| TRG-AS1 | gd.T.cells | LILRB5 | Monocyte | TRAJ25 | TCRAJ |
| BLK | gd.T.cells | LMNB1 | Monocyte | TRAJ26 | TCRAJ |
| TARP | gd.T.cells | LY86 | Monocyte | TRAJ27 | TCRAJ |
| TRDC | gd.T.cells | LYZ | Monocyte | TRAJ28 | TCRAJ |
| CD3E | gd.T.cells | MARCO | Monocyte | TRAJ29 | TCRAJ |
| CD3G | gd.T.cells | MEGF10 | Monocyte | TRAJ3 | TCRAJ |
| CD177 | Granulocyte | MERTK | Monocyte | TRAJ30 | TCRAJ |
| CLC | Granulocyte | MFGE8 | Monocyte | TRAJ31 | TCRAJ |
| CTSS | Granulocyte | MPEG1 | Monocyte | TRAJ32 | TCRAJ |
| CXCR2 | Granulocyte | MRC1 | Monocyte | TRAJ33 | TCRAJ |
| DEFA1 | Granulocyte | MS4A4A | Monocyte | TRAJ34 | TCRAJ |
| FUT7 | Granulocyte | MSR1 | Monocyte | TRAJ35 | TCRAJ |
| LTB4R | Granulocyte | NTSR1 | Monocyte | TRAJ36 | TCRAJ |
| MMP25 | Granulocyte | OCM | Monocyte | TRAJ37 | TCRAJ |
| OSM | Granulocyte | OLR1 | Monocyte | TRAJ38 | TCRAJ |
| RETN | Granulocyte | OSCAR | Monocyte | TRAJ39 | TCRAJ |
| EIF2AK2 | IFN | OTOF | Monocyte | TRAJ4 | TCRAJ |
| GBP1 | IFN | PDCD1LG2 | Monocyte | TRAJ40 | TCRAJ |
| GBP2 | IFN | PILRA | Monocyte | TRAJ41 | TCRAJ |
| GBP4 | IFN | PLA2G2D | Monocyte | TRAJ42 | TCRAJ |
| HERC5 | IFN | PLA2G5 | Monocyte | TRAJ43 | TCRAJ |
| HERC6 | IFN | PYHIN1 | Monocyte | TRAJ44 | TCRAJ |
| IFI27 | IFN | S100A8 | Monocyte | TRAJ45 | TCRAJ |
| IFI30 | IFN | S100A9 | Monocyte | TRAJ46 | TCRAJ |
| IFI35 | IFN | S1PR5 | Monocyte | TRAJ47 | TCRAJ |
| IFI44 | IFN | SCARB1 | Monocyte | TRAJ48 | TCRAJ |
| IFI44L | IFN | SCARF2 | Monocyte | TRAJ49 | TCRAJ |
| IFI6 | IFN | SECTM1 | Monocyte | TRAJ5 | TCRAJ |
| IFIT1 | IFN | SEMA4A | Monocyte | TRAJ50 | TCRAJ |
| IFIT2 | IFN | SERPINB9 | Monocyte | TRAJ52 | TCRAJ |
| IFIT3 | IFN | SERPING1 | Monocyte | TRAJ53 | TCRAJ |
| IFIT5 | IFN | SIGLEC1 | Monocyte | TRAJ54 | TCRAJ |
| IFITM1 | IFN | SIGLEC14 | Monocyte | TRAJ56 | TCRAJ |
| IFITM2 | IFN | SIGLEC5 | Monocyte | TRAJ57 | TCRAJ |
| IFITM3 | IFN | SIGLEC7 | Monocyte | TRAJ58 | TCRAJ |
| ISG15 | IFN | SLC11A1 | Monocyte | TRAJ59 | TCRAJ |
| ISG20 | IFN | SLITRK4 | Monocyte | TRAJ6 | TCRAJ |
| MX1 | IFN | SMPDL3B | Monocyte | TRAJ61 | TCRAJ |
| MX2 | IFN | SPIC | Monocyte | TRAJ7 | TCRAJ |
| OAS1 | IFN | STAB2 | Monocyte | TRAJ8 | TCRAJ |
| OAS2 | IFN | STAP2 | Monocyte | TRAJ9 | TCRAJ |
| OAS3 | IFN | TEK | Monocyte | TRBC2 | TCRB |
| OASL | IFN | TGM2 | Monocyte | TRBJ2-1 | TCRB |
| RSAD2 | IFN | TIMD4 | Monocyte | TRBJ2-2 | TCRB |
| SAMD9 | IFN | TLR2 | Monocyte | TRBJ2-2P | TCRB |
| SAMD9L | IFN | TLR8 | Monocyte | TRBJ2-3 | TCRB |
| SP100 | IFN | TNF | Monocyte | TRBJ2-4 | TCRB |
| SP110 | IFN | TNFAIP8L2 | Monocyte | TRBJ2-5 | TCRB |
| IGHA1 | IG.CHAINS | TNFRSF1B | Monocyte | TRBJ2-6 | TCRB |
| IGHA2 | IG.CHAINS | TNIP3 | Monocyte | TRBJ2-7 | TCRB |
| IGHD2-15 | IG.CHAINS | TULP1 | Monocyte | TRBV1 | TCRB |
| IGHD2-2 | IG.CHAINS | UBD | Monocyte | TRBV10-1 | TCRB |
| IGHD2-21 | IG.CHAINS | VENTX | Monocyte | TRBV10-2 | TCRB |
| IGHD3-10 | IG.CHAINS | VSTM1 | Monocyte | TRBV11-1 | TCRB |
| IGHD3-16 | IG.CHAINS | ABHD3 | Neutrophil | TRBV11-2 | TCRB |
| IGHD3-3 | IG.CHAINS | ADAM8 | Neutrophil | TRBV19 | TCRB |
| IGHD3-9 | IG.CHAINS | CD177 | Neutrophil | TRBV2 | TCRB |
| IGHG1 | IG.CHAINS | CD83 | Neutrophil | TRBV20-1 | TCRB |
| IGHG2 | IG.CHAINS | CLEC6A | Neutrophil | TRBV21-1 | TCRB |
| IGHG3 | IG.CHAINS | LY6E | Neutrophil | TRBV23-1 | TCRB |
| IGHG4 | IG.CHAINS | S100A6 | Neutrophil | TRBV24-1 | TCRB |
| IGHJ1 | IG.CHAINS | KLRF1 | NK | TRBV25-1 | TCRB |
| IGHJ2 | IG.CHAINS | NCAM1 | NK | TRBV27 | TCRB |
| IGHJ3 | IG.CHAINS | NCR1 | NK | TRBV28 | TCRB |
| IGHJ4 | IG.CHAINS | NCR3 | NK | TRBV3-1 | TCRB |
| IGHJ5 | IG.CHAINS | SH2D1B | NK | TRBV4-1 | TCRB |
| IGHJ6 | IG.CHAINS | ATP5A1 | Oxidative.Phosphorylation | TRBV4-2 | TCRB |
| IGHV1-18 | IG.CHAINS | ATP5B | Oxidative.Phosphorylation | TRBV5-1 | TCRB |
| IGHV1-2 | IG.CHAINS | ATP5D | Oxidative.Phosphorylation | TRBV5-3 | TCRB |
| IGHV1-24 | IG.CHAINS | ATP5E | Oxidative.Phosphorylation | TRBV5-4 | TCRB |
| IGHV1-3 | IG.CHAINS | ATP5F1 | Oxidative.Phosphorylation | TRBV5-5 | TCRB |
| IGHV1-45 | IG.CHAINS | ATP5G1 | Oxidative.Phosphorylation | TRBV5-6 | TCRB |
| IGHV1-46 | IG.CHAINS | ATP5G2 | Oxidative.Phosphorylation | TRBV5-7 | TCRB |
| IGHV1-58 | IG.CHAINS | ATP5G3 | Oxidative.Phosphorylation | TRBV6-1 | TCRB |
| IGHV1-69-2 | IG.CHAINS | ATP5H | Oxidative.Phosphorylation | TRBV6-4 | TCRB |
| IGHV2-26 | IG.CHAINS | ATP5I | Oxidative.Phosphorylation | TRBV6-5 | TCRB |
| IGHV2-5 | IG.CHAINS | ATP5J | Oxidative.Phosphorylation | TRBV6-6 | TCRB |
| IGHV2-70 | IG.CHAINS | ATP5J2 | Oxidative.Phosphorylation | TRBV6-7 | TCRB |
| IGHV3-13 | IG.CHAINS | ATP5L | Oxidative.Phosphorylation | TRBV6-8 | TCRB |
| IGHV3-15 | IG.CHAINS | ATP50 | Oxidative.Phosphorylation | TRBV7-1 | TCRB |
| IGHV3-20 | IG.CHAINS | ATP5S | Oxidative.Phosphorylation | TRBV7-3 | TCRB |
| IGHV3-21 | IG.CHAINS | BCS1L | Oxidative.Phosphorylation | TRBV7-4 | TCRB |
| IGHV3-23 | IG.CHAINS | CEP89 | Oxidative.Phosphorylation | TRBV7-5 | TCRB |
| IGHV3-33 | IG.CHAINS | COA1 | Oxidative.Phosphorylation | TRBV7-6 | TCRB |
| IGHV3-43 | IG.CHAINS | COA3 | Oxidative.Phosphorylation | TRBV7-7 | TCRB |
| IGHV3-48 | IG.CHAINS | COA4 | Oxidative.Phosphorylation | TRBV9 | TCRB |
| IGHV3-49 | IG.CHAINS | COA5 | Oxidative.Phosphorylation | TRDC | TCRD |
| IGHV3-53 | IG.CHAINS | COA6 | Oxidative.Phosphorylation | TRDJ1 | TCRD |
| IGHV3-62 | IG.CHAINS | COA7 | Oxidative.Phosphorylation | TRDJ2 | TCRD |
| IGHV3-64 | IG.CHAINS | COX10 | Oxidative.Phosphorylation | TRDJ3 | TCRD |
| IGHV3-7 | IG.CHAINS | COX10-AS1 | Oxidative.Phosphorylation | TRDJ4 | TCRD |
| IGHV3-72 | IG.CHAINS | COX11 | Oxidative.Phosphorylation | TRDV1 | TCRD |
| IGHV3-73 | IG.CHAINS | COX14 | Oxidative.Phosphorylation | TRDV2 | TCRD |
| IGHV3-74 | IG.CHAINS | COX15 | Oxidative.Phosphorylation | TRDV3 | TCRD |
| IGHV4-28 | IG.CHAINS | COX16 | Oxidative.Phosphorylation | ACLY | TNF_Waddel.Up |
| IGHV4-30-2 | IG.CHAINS | COX17 | Oxidative.Phosphorylation | ACSL1 | TNF_Waddel.Up |
| IGHV4-34 | IG.CHAINS | COX18 | Oxidative.Phosphorylation | ADGRE2 | TNF_Waddel.Up |
| IGHV4-39 | IG.CHAINS | COX19 | Oxidative.Phosphorylation | AK3 | TNF_Waddel.Up |
| IGHV4-59 | IG.CHAINS | COX20 | Oxidative.Phosphorylation | AKAP10 | TNF_Waddel.Up |
| IGHV4-61 | IG.CHAINS | COX4I1 | Oxidative.Phosphorylation | AMPD3 | TNF_Waddel.Up |
| IGHV5-51 | IG.CHAINS | COX412 | Oxidative.Phosphorylation | APOL3 | TNF_Waddel.Up |
| IGHV6-1 | IG.CHAINS | COX5A | Oxidative.Phosphorylation | ARID3A | TNF_Waddel.Up |
| IGHV7-81 | IG.CHAINS | COX5B | Oxidative.Phosphorylation | ARSE | TNF_Waddel.Up |
| IGKC | IG.CHAINS | COX6A1 | Oxidative.Phosphorylation | ASAP1 | TNF_Waddel.Up |
| IGKJ1 | IG.CHAINS | COX6A2 | Oxidative.Phosphorylation | B4GALT5 | TNF_Waddel.Up |
| IGKJ2 | IG.CHAINS | COX6B1 | Oxidative.Phosphorylation | BCL2A1 | TNF_Waddel.Up |
| IGKJ3 | IG.CHAINS | COX6B2 | Oxidative.Phosphorylation | BHLHE41 | TNF_Waddel.Up |
| IGKJ4 | IG.CHAINS | COX6C | Oxidative.Phosphorylation | BHMT | TNF_Waddel.Up |
| IGKJ5 | IG.CHAINS | COX7A1 | Oxidative.Phosphorylation | BIRC3 | TNF_Waddel.Up |
| IGKV1-16 | IG.CHAINS | COX7A2 | Oxidative.Phosphorylation | BRCA1 | TNF_Waddel.Up |
| IGKV1-17 | IG.CHAINS | COX7A2L | Oxidative.Phosphorylation | CALD1 | TNF_Waddel.Up |
| IGKV1-27 | IG.CHAINS | COX7B | Oxidative.Phosphorylation | CASP1 | TNF_Waddel.Up |
| IGKV1-5 | IG.CHAINS | COX7B2 | Oxidative.Phosphorylation | CASP10 | TNF_Waddel.Up |
| IGKV1-6 | IG.CHAINS | COX7C | Oxidative.Phosphorylation | CCL15 | TNF_Waddel.Up |
| IGKV1-9 | IG.CHAINS | COX8A | Oxidative.Phosphorylation | CCL20 | TNF_Waddel.Up |
| IGKV1D-16 | IG.CHAINS | COX8C | Oxidative.Phosphorylation | CCL23 | TNF_Waddel.Up |
| IGKV1D-17 | IG.CHAINS | CYC1 | Oxidative.Phosphorylation | CCL3L1 | TNF_Waddel.Up |
| IGKV1D-43 | IG.CHAINS | CYCS | Oxidative.Phosphorylation | CD37 | TNF_Waddel.Up |
| IGKV1D-8 | IG.CHAINS | DNAJC15 | Oxidative.Phosphorylation | CD38 | TNF_Waddel.Up |
| IGKV2-24 | IG.CHAINS | MT-ATP6 | Oxidative.Phosphorylation | CD83 | TNF_Waddel.Up |
| IGKV2D-26 | IG.CHAINS | MT-ATP8 | Oxidative.Phosphorylation | CDKN3 | TNF_Waddel.Up |
| IGKV2D-29 | IG.CHAINS | MT-CO1 | Oxidative.Phosphorylation | CKB | TNF_Waddel.Up |
| IGKV2D-30 | IG.CHAINS | MT-CO2 | Oxidative.Phosphorylation | CR2 | TNF_Waddel.Up |
| IGKV3-20 | IG.CHAINS | MT-CO3 | Oxidative.Phosphorylation | CTNND2 | TNF_Waddel.Up |
| IGKV3D-20 | IG.CHAINS | MT-CYB | Oxidative.Phosphorylation | CXCL1 | TNF_Waddel.Up |
| IGKV3D-7 | IG.CHAINS | MT-ND1 | Oxidative.Phosphorylation | CXCL2 | TNF_Waddel.Up |
| IGKV4-1 | IG.CHAINS | MT-ND2 | Oxidative.Phosphorylation | CXCL3 | TNF_Waddel.Up |
| IGKV5-2 | IG.CHAINS | MT-ND3 | Oxidative.Phosphorylation | CXCL8 | TNF_Waddel.Up |
| IGLC2 | IG.CHAINS | MT-ND4 | Oxidative.Phosphorylation | CYP27B1 | TNF_Waddel.Up |
| IGLC7 | IG.CHAINS | MT-ND4L | Oxidative.Phosphorylation | DAB2 | TNF_Waddel.Up |
| IGLJ6 | IG.CHAINS | MT-ND5 | Oxidative.Phosphorylation | EBI3 | TNF_Waddel.Up |
| IGLV10-54 | IG.CHAINS | MT-ND6 | Oxidative.Phosphorylation | EGR1 | TNF_Waddel.Up |
| IGLV1-36 | IG.CHAINS | NDUFA1 | Oxidative.Phosphorylation | EGR2 | TNF_Waddel.Up |
| IGLV1-40 | IG.CHAINS | NDUFA10 | Oxidative.Phosphorylation | EPB41 | TNF_Waddel.Up |
| IGLV1-47 | IG.CHAINS | NDUFA11 | Oxidative.Phosphorylation | EREG | TNF_Waddel.Up |
| IGLV2-11 | IG.CHAINS | NDUFA12 | Oxidative.Phosphorylation | ETAA1 | TNF_Waddel.Up |
| IGLV2-18 | IG.CHAINS | NDUFA13 | Oxidative.Phosphorylation | F3 | TNF_Waddel.Up |
| IGLV2-23 | IG.CHAINS | NDUFA2 | Oxidative.Phosphorylation | FABP1 | TNF_Waddel.Up |
| IGLV2-33 | IG.CHAINS | NDUFA3 | Oxidative.Phosphorylation | FBXL2 | TNF_Waddel.Up |
| IGLV2-8 | IG.CHAINS | NDUFA4 | Oxidative.Phosphorylation | FCER2 | TNF_Waddel.Up |
| IGLV3-1 | IG.CHAINS | NDUFA4L2 | Oxidative.Phosphorylation | FCGR2A | TNF_Waddel.Up |
| IGLV3-10 | IG.CHAINS | NDUFA5 | Oxidative.Phosphorylation | FLJ11129 | TNF_Waddel.Up |
| IGLV3-12 | IG.CHAINS | NDUFA6 | Oxidative.Phosphorylation | FLNA | TNF_Waddel.Up |
| IGLV3-16 | IG.CHAINS | NDUFA7 | Oxidative.Phosphorylation | GOS2 | TNF_Waddel.Up |
| IGLV3-19 | IG.CHAINS | NDUFA8 | Oxidative.Phosphorylation | GBP1 | TNF_Waddel.Up |
| IGLV3-21 | IG.CHAINS | NDUFA9 | Oxidative.Phosphorylation | GCH1 | TNF_Waddel.Up |
| IGLV3-25 | IG.CHAINS | NDUFAB1 | Oxidative.Phosphorylation | GJB2 | TNF_Waddel.Up |
| IGLV3-27 | IG.CHAINS | NDUFAF1 | Oxidative.Phosphorylation | GLS | TNF_Waddel.Up |
| IGLV3-32 | IG.CHAINS | NDUFAF2 | Oxidative.Phosphorylation | GMIP | TNF_Waddel.Up |
| IGLV4-3 | IG.CHAINS | NDUFAF3 | Oxidative.Phosphorylation | GP1BA | TNF_Waddel.Up |
| IGLV4-60 | IG.CHAINS | NDUFAF4 | Oxidative.Phosphorylation | GRK3 | TNF_Waddel.Up |
| IGLV4-69 | IG.CHAINS | NDUFAF5 | Oxidative.Phosphorylation | HCAR3 | TNF_Waddel.Up |
| IGLV5-37 | IG.CHAINS | NDUFAF6 | Oxidative.Phosphorylation | HHEX | TNF_Waddel.Up |
| IGLV5-45 | IG.CHAINS | NDUFAF7 | Oxidative.Phosphorylation | HOMER2 | TNF_Waddel.Up |
| IGLV6-57 | IG.CHAINS | NDUFAF8 | Oxidative.Phosphorylation | HP | TNF_Waddel.Up |
| IGLV7-43 | IG.CHAINS | NDUFB1 | Oxidative.Phosphorylation | ICAM1 | TNF_Waddel.Up |
| IGLV8-61 | IG.CHAINS | NDUFB10 | Oxidative.Phosphorylation | IDO1 | TNF_Waddel.Up |
| IGLV9-49 | IG.CHAINS | NDUFB11 | Oxidative.Phosphorylation | IFI44 | TNF_Waddel.Up |
| IGLVI-70 | IG.CHAINS | NDUFB2 | Oxidative.Phosphorylation | IKBKG | TNF_Waddel.Up |
| CHUK | IL1.Pathway | NDUFB2-AS1 | Oxidative.Phosphorylation | IL16 | TNF_Waddel.Up |
| IKBKB | IL1.Pathway | NDUFB3 | Oxidative.Phosphorylation | IL18 | TNF_Waddel.Up |
| NFKB1 | IL1.Pathway | NDUFB4 | Oxidative.Phosphorylation | IL1A | TNF_Waddel.Up |
| MAP2K1 | IL1.Pathway | NDUFB5 | Oxidative.Phosphorylation | IL1B | TNF_Waddel.Up |
| MAP2K4 | IL1.Pathway | NDUFB6 | Oxidative.Phosphorylation | IL1RN | TNF_Waddel.Up |
| IKBKG | IL1.Pathway | NDUFB7 | Oxidative.Phosphorylation | IL6 | TNF_Waddel.Up |
| IL1A | IL1.Pathway | NDUFB8 | Oxidative.Phosphorylation | INHBA | TNF_Waddel.Up |
| IL1R1 | IL1.Pathway | NDUFB9 | Oxidative.Phosphorylation | INSIG1 | TNF_Waddel.Up |
| IRAK1 | IL1.Pathway | NDUFC1 | Oxidative.Phosphorylation | ITGA6 | TNF_Waddel.Up |
| MAP3K3 | IL1.Pathway | NDUFC2 | Oxidative.Phosphorylation | KITLG | TNF_Waddel.Up |
| MYD88 | IL1.Pathway | NDUFS1 | Oxidative.Phosphorylation | KLF1 | TNF_Waddel.Up |
| MAP2K6 | IL1.Pathway | NDUFS2 | Oxidative.Phosphorylation | KMO | TNF_Waddel.Up |
| MAP3K7 | IL1.Pathway | NDUFS3 | Oxidative.Phosphorylation | LGALS3BP | TNF_Waddel.Up |
| TRAF6 | IL1.Pathway | NDUFS4 | Oxidative.Phosphorylation | MAP3K4 | TNF_Waddel.Up |
| TAB1 | IL1.Pathway | NDUFS5 | Oxidative.Phosphorylation | MARCKS | TNF_Waddel.Up |
| TAB2 | IL1.Pathway | NDUFS6 | Oxidative.Phosphorylation | MGLL | TNF_Waddel.Up |
| IRAK4 | IL1.Pathway | NDUFS7 | Oxidative.Phosphorylation | MMP19 | TNF_Waddel.Up |
| TOLLIP | IL1.Pathway | NDUFS8 | Oxidative.Phosphorylation | MN1 | TNF_Waddel.Up |
| MAP3K8 | IL1.Pathway | NDUFV1 | Oxidative.Phosphorylation | MRPS15 | TNF_Waddel.Up |
| IL1RAP | IL1.Pathway | NDUFV2 | Oxidative.Phosphorylation | MSC | TNF_Waddel.Up |
| IRAK2 | IL1.Pathway | NDUFV3 | Oxidative.Phosphorylation | MTF1 | TNF_Waddel.Up |
| MAPK8 | IL1.Pathway | NUBPL | Oxidative.Phosphorylation | MX1 | TNF_Waddel.Up |
| RELA | IL1.Pathway | OXA1L | Oxidative.Phosphorylation | NAMPT | TNF_Waddel.Up |
| TAB3 | IL1.Pathway | RFESD | Oxidative.Phosphorylation | NELL2 | TNF_Waddel.Up |
| IL1B | IL1.Pathway | SCO1 | Oxidative.Phosphorylation | NFKB1 | TNF_Waddel.Up |
| UBE2N | IL1.Pathway | SCO2 | Oxidative.Phosphorylation | NFKB2 | TNF_Waddel.Up |
| SQSTM1 | IL1.Pathway | SLC25A4 | Oxidative.Phosphorylation | NFKBIA | TNF_Waddel.Up |
| IRAK3 | IL1.Pathway | SURF1 | Oxidative.Phosphorylation | NFKBIZ | TNF_Waddel.Up |
| IL12B | IL23.Complex | TACO1 | Oxidative.Phosphorylation | NKX3-2 | TNF_Waddel.Up |
| IL12RB1 | IL23.Complex | TIMMDC1 | Oxidative.Phosphorylation | NR3C1 | TNF_Waddel.Up |
| IL23A | IL23.Complex | TMEM126B | Oxidative.Phosphorylation | OAS3 | TNF_Waddel.Up |
| IL23R | IL23.Complex | TRAP1 | Oxidative.Phosphorylation | PATJ | TNF_Waddel.Up |
| PSMB10 | Immunoproteasome | TTC19 | Oxidative.Phosphorylation | PDE4DIP | TNF_Waddel.Up |
| PSMB8 | Immunoproteasome | UQCC1 | Oxidative.Phosphorylation | PDPN | TNF_Waddel.Up |
| PSMB9 | Immunoproteasome | UQCC2 | Oxidative.Phosphorylation | PIAS4 | TNF_Waddel.Up |
| AIM2 | Inflammasome | UQCC3 | Oxidative.Phosphorylation | PLAUR | TNF_Waddel.Up |
| CASP1 | Inflammasome | UQCR10 | Oxidative.Phosphorylation | PTGES | TNF_Waddel.Up |
| CASP5 | Inflammasome | UQCR11 | Oxidative.Phosphorylation | PTGS2 | TNF_Waddel.Up |
| CTSB | Inflammasome | UQCRB | Oxidative.Phosphorylation | RELB | TNF_Waddel.Up |
| GSDMB | Inflammasome | UQCRC1 | Oxidative.Phosphorylation | RPGR | TNF_Waddel.Up |
| GSDMD | Inflammasome | UQCRC2 | Oxidative.Phosphorylation | RPS9 | TNF_Waddel.Up |
| NAIP | Inflammasome | UQCRFS1 | Oxidative.Phosphorylation | SDC4 | TNF_Waddel.Up |
| NEK7 | Inflammasome | UQCRH | Oxidative.Phosphorylation | SERPIND1 | TNF_Waddel.Up |
| NLRC4 | Inflammasome | UQCRHL | Oxidative.Phosphorylation | SFRP1 | TNF_Waddel.Up |
| NLRP1 | Inflammasome | UQCRQ | Oxidative.Phosphorylation | SH3BP5 | TNF_Waddel.Up |
| NLRP3 | Inflammasome | CLEC4C | pDC | SLAMF1 | TNF_Waddel.Up |
| NOD2 | Inflammasome | NRP1 | pDC | SLC30A4 | TNF_Waddel.Up |
| P2RX7 | Inflammasome | IL3RA | pDC | SOD2 | TNF_Waddel.Up |
| PANX1 | Inflammasome | CLEC4C | pDC | SPI1 | TNF_Waddel.Up |
| PYCARD | Inflammasome | NRP1 | pDC | SSPN | TNF_Waddel.Up |
| RIPK1 | Inflammasome | C19orf10 | Plasma.Cells | STAT4 | TNF_Waddel.Up |
| IL1A | Inflammatory.Cytokines | IGH | Plasma.Cells | TAF15 | TNF_Waddel.Up |
| CXCL10 | Inflammatory.Cytokines | IGHD | Plasma.Cells | TAP2 | TNF_Waddel.Up |
| CXCL11 | Inflammatory.Cytokines | IGHG1 | Plasma.Cells | TBX3 | TNF_Waddel.Up |
| CXCL9 | Inflammatory.Cytokines | IGHMBP2 | Plasma.Cells | TFF1 | TNF_Waddel.Up |
| TNFSF13B | Inflammatory.Cytokines | IGHV2-5 | Plasma.Cells | TNF | TNF_Waddel.Up |
| CCL8 | Inflammatory.Cytokines | IGHV3-20 | Plasma.Cells | TNFAIP2 | TNF_Waddel.Up |
| TNF | Inflammatory.Cytokines | IGHV3-23 | Plasma.Cells | TNFAIP3 | TNF_Waddel.Up |
| IL1B | Inflammatory.Cytokines | IGHV4-28 | Plasma.Cells | TNFRSF11A | TNF_Waddel.Up |
| IL18 | Inflammatory.Cytokines | IGHV4-31 | Plasma.Cells | TRAF1 | TNF_Waddel.Up |
| AOAH | Inhibitory.Macs | IGHV4-34 | Plasma._Cells | TSC22D1 | TNF_Waddel.Up |
| BACH1 | Inhibitory.Macs | IGK | Plasma.Cells | TYROBP | TNF_Waddel.Up |
| CD200R1 | Inhibitory.Macs | IGKC | Plasma.Cells | UBE2C | TNF_Waddel.Up |
| CD300A | Inhibitory.Macs | IGL | Plasma.Cells | VEGFA | TNF_Waddel.Up |
| CD163 | Inhibitory.Macs | IGLJ3 | Plasma.Cells | WT1 | TNF_Waddel.Up |
| CLEC7A | Inhibitory.Macs | IGLL1 | Plasma.Cells | B4GALT3 | Unfolded.Protein |
| SCARB1 | Inhibitory.Macs | IGLV@ | Plasma.Cells | CALR | Unfolded.Protein |
| MS4A4A | Inhibitory.Macs | IGLV1-40 | Plasma.Cells | CALU | Unfolded.Protein |
| IL10 | Inhibitory.Macs | IGLV1-44 | Plasma.Cells | CANX | Unfolded.Protein |
| AZU1 | LDG | IGLV2-14 | Plasma.Cells | CDS2 | Unfolded.Protein |
| CAMP | LDG | IGLV2-5 | Plasma.Cells | CHST12 | Unfolded.Protein |
| CEACAM6 | LDG | IGLV3-1 | Plasma.Cells | CHST2 | Unfolded.Protein |
| CEACAM8 | LDG | IGLV3-19 | Plasma.Cells | DERL1 | Unfolded.Protein |
| CTSG | LDG | IGLV3-25 | Plasma.Cells | DERL2 | Unfolded.Protein |
| DEFA4 | LDG | IGLV4-3 | Plasma.Cells | DNAJC3 | Unfolded.Protein |
| ELANE | LDG | IGLV4-60 | Plasma.Cells | EDEM2 | Unfolded.Protein |
| LCN2 | LDG | IGLV5-45 | Plasma.Cells | EDEM3 | Unfolded.Protein |
| LTF | LDG | IGLV6-57 | Plasma.Cells | EMC9 | Unfolded.Protein |
| MPO | LDG | IGLVI-70 | Plasma.Cells | ERAP1 | Unfolded.Protein |
| OLFM4 | LDG | MZB1 | Plasma.Cells | ERGIC2 | Unfolded.Protein |
| RNASE3 | LDG | PRDM1 | Plasma.Cells | ERO1L | Unfolded.Protein |
| HLA-DMA | MHC.II | SDC1 | Plasma.Cells | EXT1 | Unfolded.Protein |
| HLA-DMB | MHC.II | THEMIS2 | Plasma.Cells | GALNT2 | Unfolded.Protein |
| HLA-DPA1 | MHC.II | TNFRSF17 | Plasma.Cells | GOLT1B | Unfolded.Protein |
| HLA-DPB1 | MHC.II | CEACAM1 | SNOR.LOW.Up | HERPUD1 | Unfolded.Protein |
| HLA-DPB2 | MHC.II | FCGR1A | SNOR.LOW.Up | HYOU1 | Unfolded.Protein |
| HLA-DQA1 | MHC.II | LGALS1 | SNOR.LOW.Up | IER3IP1 | Unfolded.Protein |
| HLA-DQA2 | MHC.II | SNORD24 | SNOR.LOW.Up | IMPAD1 | Unfolded.Protein |
| HLA-DQB1 | MHC.II | SNORD44 | SNOR.LOW.Up | KDELC1 | Unfolded.Protein |
| HLA-DQB2 | MHC.II | SNORD47 | SNOR.LOW.Up | KDELR2 | Unfolded.Protein |
| HLA-DRA | MHC.II | SNORD80 | SNOR.LOW.Up | LMAN2 | Unfolded.Protein |
| HLA-DRB1 | MHC.II | CCR3 | T.Cells | LPGAT1 | Unfolded.Protein |
| HLA-DRB3 | MHC.II | CD226 | T.Cells | MAN1A1 | Unfolded.Protein |
| HLA-DRB4 | MHC.II | CD247 | T.Cells | MANEA | Unfolded.Protein |
| HLA-DRB5 | MHC.II | CD28 | T.Cells | MANF | Unfolded.Protein |
| HLA-DRB6 | MHC.II | CD3D | T.Cells | NUCB2 | Unfolded.Protein |
| ACE | Monocyte | CD3E | T.Cells | PDIA4 | Unfolded.Protein |
| ADAM8 | Monocyte | CD3G | T.Cells | PDIA6 | Unfolded.Protein |
| ADAMDEC1 | Monocyte | CD4 | T.Cells | PIGK | Unfolded.Protein |
| ADGRE1 | Monocyte | CD5 | T.Cells | PPIB | Unfolded.Protein |
| ADGRE2 | Monocyte | CD8A | T.Cells | SEC24D | Unfolded.Protein |
| APOBEC3B | Monocyte | CD8B | T.Cells | SEC61G | Unfolded.Protein |
| APOBEC3G | Monocyte | ETS1 | T.Cells | SPCS3 | Unfolded.Protein |
| APOBR | Monocyte | GATA3 | T.Cells | SSR1 | Unfolded.Protein |
| ART4 | Monocyte | GRAP2 | T.Cells | SSR3 | Unfolded.Protein |
| C1QA | Monocyte | LEF1 | T.Cells | TRAM1 | Unfolded.Protein |
| C1QC | Monocyte | SH2D1A | T.Cells | TRAM2 | Unfolded.Protein |
| C2 | Monocyte | TRAC | T.Cells | UGGT1 | Unfolded.Protein |
| C4A | Monocyte | TRBC1 | T.Cells | XBP1 | Unfolded.Protein |
| C4B | Monocyte | TRDC | T.Cells | | |
| C4BPA | Monocyte | FOXP3 | T.reg | | |

### Example 5: Molecular Endotypes of RA Patient Synovium Sample

A method was carried out to characterize the molecular landscape of patients with rheumatoid arthritis (RA) by analyzing gene expression profiles from synovium samples. Synovium samples were collected from a patient population comprising patients with rheumatoid arthritis (RA) that are incomplete responders (IR) to DMARD treatment (RA DMARD-IR patients) treated with TNFi.

Full transcriptomic RNA sequencing was carried out on synovium samples collected from a patient population. The RNA sequencing assay involved 46 RA DMARD-IR patients treated with TNFi.

In brief, synovium samples were collected. After removal of ribosomal RNA and globin transcripts with the Ribo-Zero Globin Removal kit (Illumina), stranded libraries are prepared with the TruSeq Library prep kit (Illumina) and hybridized to a flow cell for sequencing with the Illumina HiSeq platform. Raw RNAseq output counts are log 2 normalized using the R DESeq2 package. The top 5,000 row variance (top5k rowVar) genes determined using standard deviation between samples were retained for further analysis.

The top 5,000 row variance genes were analyzed by a suite of gene expression technologies, including Multiscale Embedded Gene Co-expression Network Analysis (MEGENA) to generate gene coexpression modules which were functionally annotated and correlated to various demographic traits, clinical features, and laboratory assays.

In brief, the MEGENA R package was used to generate a gene coexpression network by inputting the top5k rowVar genes. MEGENA multi-scale clustering analysis (MCA) formed lineages of gene modules followed by identification of densely intraconnected hub genes using multi-scale hub analysis (MHA). Modules were assigned “lineage” names based on their multiscale pedigree from the root MEGENA module. The prcomp package was utilized to perform singular value decomposition and calculate MEGENA module eigengenes (MEs), equivalent to the first principal component calculated amongst the variance of a given MEGENA module. MEGENA MEs were correlated to the numerically encoded sample traits.

A heatmap was generated using ComplexHeatmap visualizing the top 40 sample trait correlations to the MEGENA modules that were significantly correlated to cohort (or cluster.) Module gene symbols were used to programmatically query the STRING database and calculate the percentage of genes within a given module predicted to have known protein-protein interactions (PPI) ranging from 0 to 100%.

A gene set variation analysis (GSVA) (GSVA (V1.25.0) R software package) was carried out as a non-parametric, unsupervised method for estimating the variation of pre-defined gene sets over all MEGENA module log 2 gene expression values. Input genes were employed only if the interquartile range (IQR) of their expression across the samples was greater than 0. Enrichment scores (GSVA scores) were calculated non-parametrically using a Kolmogorov Smirnoff (KS)-like random walk statistic. The enrichment scores(ES) were the largest positive and negative random walk deviations from zero, respectively, for a particular sample amongst the module gene set. The GSVA scores were used as input for unsupervised stable k-means clustering, and five different disease phenotypes or clusters were identified (i.e., cluster 0, cluster 1, cluster 2, cluster 3, cluster 4). GSVA was performed using the significant MEGENA modules as gene signatures.

The MEs of the significant MEGENA modules were correlated to mean gene expression of a given module per patient and visualized using Complex heatmap. As shown in FIG. 11, columns of patients with RA that are TNF-IR were clustered using idealized k-means clustering on patient samples and three different disease phenotypes or clusters were identified (i.e., cluster 1, cluster 2, cluster 3). Also as shown in FIG. 11, rows correspond to various immune cell type and process modules (e.g., Synovium modules: A, B, C, D, E, F, G, H, I, J, K, and L) used to identify the three patient subsets. In brief, the modules, can be associated to subsets with enriched treatment targets. Overall, stable k-means clustering of gene coexpression modules effectively segregated patient subsets into three different disease phenotypes which may be associated to different treatment targets and/or responsive to different treatments. Overall, FIG. 11 shows clinical response of RA DMARD-IR patients treated with TNFi. As shown in FIG. 11A, rows correspond to various EULAR response criteria (i.e., subject trait or patient trait), used to classify individual patients as non-responders, moderate responders, or high responders, depending on the extent of change and the level of disease activity reached. As shown in FIG. 11A, rows visualize in color (color choice arbitrarily selected to ease visualization) whether the EULAR response criteria (e.g., patient traits or subject traits: patient is female (pt.is.female), patient ancestry (pt.anc.), patient ancestry African (pt.anc.AA), patient ancestry Asian (pt.anc.AsA), patient ancestry Caribean (pt.anc.Carib), patient ancestry European (pt.anc.EA), patient treatment certolizumab pegol (CZP) (pt.treatment.CZP), patient treatment ETC (pt.treatment.ETC), patient non responder (patient.responder.non), patient moderate responder (patient.responder.mod), patient high responder (patient.responder.high), patient pauci-immune designation (pt.histo.pauci.immune), patient biopsy from wrist (pt.biop.wrist), patient biopsy from knee (pt.biop.knee), patient indication of RA erosion (pt.ra.erosion), patient pathology myeloid (pt.patho.myeloid), patient pathology lymphoid (pt.patho.lymphoid), patient pathology fibroid (pt.patho.fibroid), etc.) was met. Gradations of color intensity, from low color intensity to high color intensity, indicates degree to which criteria was met (e.g., continuous values: patient age (pt.age), patient inflammatory score (pt.score.inf), patient DAS28 score (pt.score.das28), patient change in DAS28 score after treatment (pt.score.das28.delta), patient HAQ score disease index (pt.score.haq.di), patient disease duration (pt.disease.duration), number of swollen joints (pt.count.joint.swollen), number of tender joints (pt.count.joint.tender), CRP C-reactive protein level (pt.CRP), patient erythrocyte sedimentation rate (ESR pt.ESR), patient rheumatoid factor (pt.RF), patient ACPA level (pt.ACPA), patient blood sample RNA concentration (pt.RNA.concentration), patient blood sample total RNA volume (pt.RNA.volume), patient blood sample total RNA yield (pt.RNA.yield), etc.). Also as shown in FIG. 11A, no color intensity shows DMARD-IR patients did not respond to TNFi treatment, low color intensity shows DMARD-IR patients were moderate responders to TNFi treatment, high color intensity shows DMARD-IR patients were high responders to TNFi treatment. Overall, the heatmap in FIG. 11A (see dark green at the bottom) shows high responders to TNFi identified in Cluster 1, as compared to low responders to TNFi identified in Cluster 3.

As shown in FIG. 11B, the heatmap color intensity represents the enrichment of gene signature, with bright red indicating increased enrichment and blue decreased enrichment, and white representing the intermediate levels. In brief, a high GSVA score (bright red) indicates a high response to TNFi therapy. Also as shown in both FIG. 11B and FIG. 11C, rows correspond to various immune cell type and process modules (e.g., Synovium modules: A, B, C, D, E, F, G, H, I, J, K, and L) used to identify the patient subsets. The columns in FIG. 11C correspond to various clinical criteria. Overall, FIG. 11C details correlations between the various immune cell type and process modules (Synovium modules) and the clinical criteria. As shown in FIG. 11C, rows visualize correlations in color, ranging from negative (blue, or 0) to positive (red, or 1), with white representing intermediate. Table 4 details the genes within the Synovium modules.

TABLE 4
Genes within the Synovium modules.

| Gene | Category | Gene | Category | Gene | Category |
|---|---|---|---|---|---|
| PRF1 | Activated T Cells | DUSP7 | IFN Beta Up | GABRB2 | Phagocytic |
| | | | | | macrophages |
| IFNG | Activated T Cells | DYNLT1 | IFN Beta Up | GNPNAT1 | Phagocytic |
| | | | | | macrophages |
| EOMES | Activated T Cells | DYSF | IFN Beta Up | HTRA1 | Phagocytic |
| | | | | | macrophages |
| TBX21 | Activated T Cells | E2F1 | IFN Beta Up | ITGAE | Phagocytic |
| | | | | | macrophages |
| GZMH | Activated T Cells | ECE1 | IFN Beta Up | ITGB5 | Phagocytic |
| | | | | | macrophages |
| CD69 | Activated T Cells | EDN1 | IFN Beta Up | KAL1 | Phagocytic |
| | | | | | macrophages |
| IL2RB | Activated T Cells | EGR1 | IFN Beta Up | KANK2 | Phagocytic |
| | | | | | macrophages |
| ZNF683 | Activated T Cells | EIF2B1 | IFN Beta Up | LAMC1 | Phagocytic |
| | | | | | macrophages |
| SGK1 | Activated T Cells | ELF1 | IFN Beta Up | LGI2 | Phagocytic |
| | | | | | macrophages |
| TFRC | Activated T Cells | ELF4 | IFN Beta Up | LZTR1 | Phagocytic |
| | | | | | macrophages |
| TAGAP | Activated T Cells | ENPP2 | IFN Beta Up | MLF1 | Phagocytic |
| | | | | | macrophages |
| GZMB | Activated T Cells | EPB41 | IFN Beta Up | NAT9 | Phagocytic |
| | | | | | macrophages |
| RELT | Activated T Cells | ETV4 | IFN Beta Up | OLFML3 | Phagocytic |
| | | | | | macrophages |
| RNF125 | Activated T Cells | ETV6 | IFN Beta Up | PROS1 | Phagocytic |
| | | | | | macrophages |
| SATB1 | Activated T Cells | F8 | IFN Beta Up | PTGFRN | Phagocytic |
| | | | | | macrophages |
| ZC3H12D | Activated T Cells | FAF1 | IFN Beta Up | PTPRM | Phagocytic |
| | | | | | macrophages |
| NKG7 | Activated T Cells | FAM65B | IFN Beta Up | RAB11FIP5 | Phagocytic |
| | | | | | macrophages |
| CD40 | Activated T Cells | FBXW2 | IFN Beta Up | RAE1 | Phagocytic |
| | | | | | macrophages |
| ICOS | Activated T Cells | FCGR1A | IFN Beta Up | RGL3 | Phagocytic |
| | | | | | macrophages |
| ZAP70 | Activated T Cells | FCMR | IFN Beta Up | RND3 | Phagocytic |
| | | | | | macrophages |
| IKZF1 | Activated T Cells | FGF1 | IFN Beta Up | RRAGA | Phagocytic |
| | | | | | macrophages |
| IKZF3 | Activated T Cells | FLNA | IFN Beta Up | ST5 | Phagocytic |
| | | | | | macrophages |
| AOAH | Anti-inflammation | FMR1 | IFN Beta Up | THRB | Phagocytic |
| | | | | | macrophages |
| TNFAIP3 | Anti-inflammation | FOXO1 | IFN Beta Up | TMEM119 | Phagocytic |
| | | | | | macrophages |
| SOCS3 | Anti-inflammation | FPR2 | IFN Beta Up | TUBB2A | Phagocytic |
| | | | | | macrophages |
| CD74 | Antigen Presentation | FTL | IFN Beta Up | TXNRD2 | Phagocytic |
| | | | | | macrophages |
| HLA-DMA | Antigen Presentation | FUT4 | IFN Beta Up | UNC5B | Phagocytic |
| | | | | | macrophages |
| HLA-DMB | Antigen Presentation | GADD45B | IFN Beta Up | VANGL1 | Phagocytic |
| | | | | | macrophages |
| HLA-DPA1 | Antigen Presentation | GBAP1 | IFN Beta Up | WDR12 | Phagocytic |
| | | | | | macrophages |
| HLA-DPB1 | Antigen Presentation | GBP1 | IFN Beta Up | ZBTB7C | Phagocytic |
| | | | | | macrophages |
| HLA-DQB1 | Antigen Presentation | GBP2 | IFN Beta Up | CD81 | PI3K Signaling in B |
| | | | | | Lymphocytes |
| HLA-DQB2 | Antigen Presentation | GCH1 | IFN Beta Up | IRS1 | PI3K Signaling in B |
| | | | | | Lymphocytes |
| HLA-DRA | Antigen Presentation | GCNT1 | IFN Beta Up | PLEKHA1 | PI3K Signaling in B |
| | | | | | Lymphocytes |
| HLA-DRB1 | Antigen Presentation | GLS | IFN Beta Up | PLEKHA3 | PI3K Signaling in B |
| | | | | | Lymphocytes |
| HLA-DRB6 | Antigen Presentation | GMPR | IFN Beta Up | PLEKHA4 | PI3K Signaling in B |
| | | | | | Lymphocytes |
| RFX5 | Antigen Presentation | GPI | IFN Beta Up | CBL | PI3K Signaling in B |
| | | | | | Lymphocytes |
| BCL10 | B Cell Receptor Signaling | GPR161 | IFN Beta Up | INPP5D | PI3K Signaling in B |
| | | | | | Lymphocytes |
| BCL2L1 | B Cell Receptor Signaling | GUK1 | IFN Beta Up | PTEN | PI3K Signaling in B |
| | | | | | Lymphocytes |
| CALM1 | B Cell Receptor Signaling | HCAR3 | IFN Beta Up | AKT2 | PI3K Signaling in B |
| | | | | | Lymphocytes |
| CALM2 | B Cell Receptor Signaling | HHEX | IFN Beta Up | AKT3 | PI3K Signaling in B |
| | | | | | Lymphocytes |
| CALM3 | B Cell Receptor Signaling | HIST2H2AA3 | IFN Beta Up | ATF1 | PI3K Signaling in B |
| | | | | | Lymphocytes |
| CDC42 | B Cell Receptor Signaling | HK2 | IFN Beta Up | ATF2 | PI3K Signaling in B |
| | | | | | Lymphocytes |
| CFL1 | B Cell Receptor Signaling | HLA-DOA | IFN Beta Up | ATF4 | PI3K Signaling in B |
| | | | | | Lymphocytes |
| CREB1 | B Cell Receptor Signaling | HS6ST1 | IFN Beta Up | ATF5 | PI3K Signaling in B |
| | | | | | Lymphocytes |
| DAPP1 | B Cell Receptor Signaling | HSP90AA1 | IFN Beta Up | ATF6 | PI3K Signaling in B |
| | | | | | Lymphocytes |
| EGR1 | B Cell Receptor Signaling | HSPAIL | IFN Beta Up | ATF7 | PI3K Signaling in B |
| | | | | | Lymphocytes |
| FCGR2B | B Cell Receptor Signaling | IDO1 | IFN Beta Up | BCL10 | PI3K Signaling in B |
| | | | | | Lymphocytes |
| FCGR2C | B Cell Receptor Signaling | IFI16 | IFN Beta Up | BLNK | PI3K Signaling in B |
| | | | | | Lymphocytes |
| FGFR1 | B Cell Receptor Signaling | IFI27 | IFN Beta Up | BTK | PI3K Signaling in B |
| | | | | | Lymphocytes |
| GRB2 | B Cell Receptor Signaling | IFI35 | IFN Beta Up | CALM1 | PI3K Signaling in B |
| | | | | | Lymphocytes |
| IGHM | B Cell Receptor Signaling | IFI44 | IFN Beta Up | CALM2 | PI3K Signaling in B |
| | | | | | Lymphocytes |
| INPP5D | B Cell Receptor Signaling | IFI6 | IFN Beta Up | CALM3 | PI3K Signaling in B |
| | | | | | Lymphocytes |
| KRAS | B Cell Receptor Signaling | IFIT1 | IFN Beta Up | CAMK2D | PI3K Signaling in B |
| | | | | | Lymphocytes |
| LYN | B Cell Receptor Signaling | IFIT5 | IFN Beta Up | CAMK2G | PI3K Signaling in B |
| | | | | | Lymphocytes |
| MAP2K6 | B Cell Receptor Signaling | IFITM1 | IFN Beta Up | CD19 | PI3K Signaling in B |
| | | | | | Lymphocytes |
| MAP3K1 | B Cell Receptor Signaling | IFITM2 | IFN Beta Up | CD40 | PI3K Signaling in B |
| | | | | | Lymphocytes |
| MAP3K2 | B Cell Receptor Signaling | IFITM3 | IFN Beta Up | CHP1 | PI3K Signaling in B |
| | | | | | Lymphocytes |
| MAP3K7 | B Cell Receptor Signaling | IFNG | IFN Beta Up | CREB1 | PI3K Signaling in B |
| | | | | | Lymphocytes |
| MAPK1 | B Cell Receptor Signaling | IFRD1 | IFN Beta Up | DAPP1 | PI3K Signaling in B |
| | | | | | Lymphocytes |
| MAPK3 | B Cell Receptor Signaling | IKBKE | IFN Beta Up | ELK1 | PI3K Signaling in B |
| | | | | | Lymphocytes |
| MAPK14 | B Cell Receptor Signaling | IKBKG | IFN Beta Up | HRAS | PI3K Signaling in B |
| | | | | | Lymphocytes |
| MEF2C | B Cell Receptor Signaling | IL15 | IFN Beta Up | IKBKB | PI3K Signaling in B |
| | | | | | Lymphocytes |
| NFKB2 | B Cell Receptor Signaling | IL15RA | IFN Beta Up | IKBKE | PI3K Signaling in B |
| | | | | | Lymphocytes |
| PAG1 | B Cell Receptor Signaling | IL18BP | IFN Beta Up | ITPR1 | PI3K Signaling in B |
| | | | | | Lymphocytes |
| PIK3AP1 | B Cell Receptor Signaling | IL18R1 | IFN Beta Up | ITPR2 | PI3K Signaling in B |
| | | | | | Lymphocytes |
| PIK3C2A | B Cell Receptor Signaling | IL1RN | IFN Beta Up | JUN | PI3K Signaling in B |
| | | | | | Lymphocytes |
| PIK3CB | B Cell Receptor Signaling | IL6 | IFN Beta Up | KRAS | PI3K Signaling in B |
| | | | | | Lymphocytes |
| PIK3CD | B Cell Receptor Signaling | IL7 | IFN Beta Up | LYN | PI3K Signaling in B |
| | | | | | Lymphocytes |
| PIK3CG | B Cell Receptor Signaling | INPP5D | IFN Beta Up | MAP2K1 | PI3K Signaling in B |
| | | | | | Lymphocytes |
| PIK3R1 | B Cell Receptor Signaling | INPPL1 | IFN Beta Up | MAP2K2 | PI3K Signaling in B |
| | | | | | Lymphocytes |
| PIK3R4 | B Cell Receptor Signaling | IRF1 | IFN Beta Up | MAPK1 | PI3K Signaling in B |
| | | | | | Lymphocytes |
| PPP3R1 | B Cell Receptor Signaling | IRF2 | IFN Beta Up | MAPK3 | PI3K Signaling in B |
| | | | | | Lymphocytes |
| PRKCB | B Cell Receptor Signaling | IRF4 | IFN Beta Up | NFAT5 | PI3K Signaling in B |
| | | | | | Lymphocytes |
| PTK2B | B Cell Receptor Signaling | IRF7 | IFN Beta Up | NFATC1 | PI3K Signaling in B |
| | | | | | Lymphocytes |
| PTPRC | B Cell Receptor Signaling | IRF9 | IFN Beta Up | NFATC2 | PI3K Signaling in B |
| | | | | | Lymphocytes |
| RAC1 | B Cell Receptor Signaling | ISG15 | IFN Beta Up | NFATC3 | PI3K Signaling in B |
| | | | | | Lymphocytes |
| RAC2 | B Cell Receptor Signaling | ISG20 | IFN Beta Up | NFATC4 | PI3K Signaling in B |
| | | | | | Lymphocytes |
| RAP1B | B Cell Receptor Signaling | ITGAL | IFN Beta Up | NFKB2 | PI3K Signaling in B |
| | | | | | Lymphocytes |
| RAP2B | B Cell Receptor Signaling | ITGAX | IFN Beta Up | NFKBIA | PI3K Signaling in B |
| | | | | | Lymphocytes |
| RPS6KB1 | B Cell Receptor Signaling | JAK2 | IFN Beta Up | NFKBIE | PI3K Signaling in B |
| | | | | | Lymphocytes |
| SOS2 | B Cell Receptor Signaling | JCHAIN | IFN Beta Up | NRAS | PI3K Signaling in B |
| | | | | | Lymphocytes |
| SYK | B Cell Receptor Signaling | JUP | IFN Beta Up | PDPK1 | PI3K Signaling in B |
| | | | | | Lymphocytes |
| BANK1 | B cells | KCNA3 | IFN Beta Up | PIK3AP1 | PI3K Signaling in B |
| | | | | | Lymphocytes |
| FCRL3 | B cells | KCNMB1 | IFN Beta Up | PIK3CB | PI3K Signaling in B |
| | | | | | Lymphocytes |
| FCRL5 | B cells | KDELR2 | IFN Beta Up | PIK3CD | PI3K Signaling in B |
| | | | | | Lymphocytes |
| CD79B | B cells | KIF20B | IFN Beta Up | PIK3CG | PI3K Signaling in B |
| | | | | | Lymphocytes |
| HLA-DOB | B cells | KLF2 | IFN Beta Up | PIK3R1 | PI3K Signaling in B |
| | | | | | Lymphocytes |
| IGH | B cells | KLF6 | IFN Beta Up | PLCB1 | PI3K Signaling in B |
| | | | | | Lymphocytes |
| LINC00926 | B cells | KLRB1 | IFN Beta Up | PLCB4 | PI3K Signaling in B |
| | | | | | Lymphocytes |
| MICAL3 | B cells | KPNB1 | IFN Beta Up | PLCD4 | PI3K Signaling in B |
| | | | | | Lymphocytes |
| MS4A1 | B cells | KRT8 | IFN Beta Up | PLCE1 | PI3K Signaling in B |
| | | | | | Lymphocytes |
| PAX5 | B cells | LAG3 | IFN Beta Up | PLCG1 | PI3K Signaling in B |
| | | | | | Lymphocytes |
| CD19 | B cells | LAMP3 | IFN Beta Up | PLCG2 | PI3K Signaling in B |
| | | | | | Lymphocytes |
| HLA-DOA | B cells | LANCL1 | IFN Beta Up | PLCL2 | PI3K Signaling in B |
| | | | | | Lymphocytes |
| ELF1 | B cells | LAP3 | IFN Beta Up | PPP3CA | PI3K Signaling in B |
| | | | | | Lymphocytes |
| BLNK | B cells | LBR | IFN Beta Up | PPP3CB | PI3K Signaling in B |
| | | | | | Lymphocytes |
| BTK | B cells | LEPR | IFN Beta Up | PPP3R1 | PI3K Signaling in B |
| | | | | | Lymphocytes |
| CD37 | B cells | LGALS2 | IFN Beta Up | PRKCB | PI3K Signaling in B |
| | | | | | Lymphocytes |
| FCRLB | B cells | LGALS3BP | IFN Beta Up | PRKCI | PI3K Signaling in B |
| | | | | | Lymphocytes |
| WDFY4 | B cells | LGALS9 | IFN Beta Up | PTPRC | PI3K Signaling in B |
| | | | | | Lymphocytes |
| ACTR2 | CCR2+ARG1+ | LGMN | IFN Beta Up | RAC1 | PI3K Signaling in B |
| | infiltrating macrophages | | | | Lymphocytes |
| | STA | | | | |
| AGPAT4 | CCR2+ARG1+ | LILRA1 | IFN Beta Up | RAF1 | PI3K Signaling in B |
| | infiltrating macrophages | | | | Lymphocytes |
| | STA | | | | |
| AGPAT5 | CCR2+ARG1+ | LINC00597 | IFN Beta Up | RRAS | PI3K Signaling in B |
| | infiltrating macrophages | | | | Lymphocytes |
| | STA | | | | |
| AK6 | CCR2+ARG1+ | LMNB1 | IFN Beta Up | SYK | PI3K Signaling in B |
| | infiltrating macrophages | | | | Lymphocytes |
| | STA | | | | |
| ARF6 | CCR2+ARG1+ | LMO2 | IFN Beta Up | TLR4 | PI3K Signaling in B |
| | infiltrating macrophages | | | | Lymphocytes |
| | STA | | | | |
| ATP5G1 | CCR2+ARG1+ | LTA | IFN Beta Up | VAV2 | PI3K Signaling in B |
| | infiltrating macrophages | | | | Lymphocytes |
| | STA | | | | |
| ATP6VIA | CCR2+ARG1+ | LTB4R | IFN Beta Up | FOXO1 | PI3K/AKT Signaling |
| | infiltrating macrophages | | | | |
| | STA | | | | |
| ATP6V1B2 | CCR2+ARG1+ | LY6E | IFN Beta Up | GAB1 | PI3K/AKT Signaling |
| | infiltrating macrophages | | | | |
| | STA | | | | |
| ATP6V1C1 | CCR2+ARG1+ | LYN | IFN Beta Up | LIMS1 | PI3K/AKT Signaling |
| | infiltrating macrophages | | | | |
| | STA | | | | |
| AVPI1 | CCR2+ARG1+ | MAP2K5 | IFN Beta Up | MCL1 | PI3K/AKT Signaling |
| | infiltrating macrophages | | | | |
| | STA | | | | |
| AZIN1 | CCR2+ARG1+ | MAP3K8 | IFN Beta Up | TP53 | PI3K/AKT Signaling |
| | infiltrating macrophages | | | | |
| | STA | | | | |
| B4GALT1 | CCR2+ARG1+ | MARCKS | IFN Beta Up | BAD | PI3K/AKT Signaling |
| | infiltrating macrophages | | | | |
| | STA | | | | |
| CALM1 | CCR2+ARG1+ | MBNL | IFN Beta Up | GSK3B | PI3K/AKT Signaling |
| | infiltrating macrophages | | | | |
| | STA | | | | |
| CCT2 | CCR2+ARG1+ | MCL1 | IFN Beta Up | INPP5B | PI3K/AKT Signaling |
| | infiltrating macrophages | | | | |
| | STA | | | | |
| CCT3 | CCR2+ARG1+ | MEF2A | IFN Beta Up | INPP5D | PI3K/AKT Signaling |
| | infiltrating macrophages | | | | |
| | STA | | | | |
| CCT8 | CCR2+ARG1+ | MFHAS1 | IFN Beta Up | INPP5F | PI3K/AKT Signaling |
| | infiltrating macrophages | | | | |
| | STA | | | | |
| CD44 | CCR2+ARG1+ | MGLL | IFN Beta Up | INPPL1 | PI3K/AKT Signaling |
| | infiltrating macrophages | | | | |
| | STA | | | | |
| CITED2 | CCR2+ARG1+ | MIG | IFN Beta Up | MAP3K5 | PI3K/AKT Signaling |
| | infiltrating macrophages | | | | |
| | STA | | | | |
| CXCL3 | CCR2+ARG1+ | MNDA | IFN Beta Up | OCRL | PI3K/AKT Signaling |
| | infiltrating macrophages | | | | |
| | STA | | | | |
| DNAJC2 | CCR2+ARG1+ | MRPS15 | IFN Beta Up | PIK3R1 | PI3K/AKT Signaling |
| | infiltrating macrophages | | | | |
| | STA | | | | |
| EIF2S2 | CCR2+ARG1+ | MS4A7 | IFN Beta Up | PPM1L | PI3K/AKT Signaling |
| | infiltrating macrophages | | | | |
| | STA | | | | |
| EIF4G2 | CCR2+ARG1+ | MSR1 | IFN Beta Up | PPP2CB | PI3K/AKT Signaling |
| | infiltrating macrophages | | | | |
| | STA | | | | |
| EIF6 | CCR2+ARG1+ | MX1 | IFN Beta Up | PPP2R2A | PI3K/AKT Signaling |
| | infiltrating macrophages | | | | |
| | STA | | | | |
| EMC6 | CCR2+ARG1+ | MX2 | IFN Beta Up | PPP2R5C | PI3K/AKT Signaling |
| | infiltrating macrophages | | | | |
| | STA | | | | |
| ESD | CCR2+ARG1+ | MYD88 | IFN Beta Up | PPP2R5E | PI3K/AKT Signaling |
| | infiltrating macrophages | | | | |
| | STA | | | | |
| EZR | CCR2+ARG1+ | NAMPT | IFN Beta Up | PTEN | PI3K/AKT Signaling |
| | infiltrating macrophages | | | | |
| | STA | | | | |
| FBL | CCR2+ARG1+ | NAPSA | IFN Beta Up | RAF1 | PI3K/AKT Signaling |
| | infiltrating macrophages | | | | |
| | STA | | | | |
| FCF1 | CCR2+ARG1+ | NBN | IFN Beta Up | SFN | PI3K/AKT Signaling |
| | infiltrating macrophages | | | | |
| | STA | | | | |
| G3BP1 | CCR2+ARG1+ | NCOA2 | IFN Beta Up | SYNJ2 | PI3K/AKT Signaling |
| | infiltrating macrophages | | | | |
| | STA | | | | |
| GNA13 | CCR2+ARG1+ | NEBL | IFN Beta Up | THEM4 | PI3K/AKT Signaling |
| | infiltrating macrophages | | | | |
| | STA | | | | |
| GNL3 | CCR2+ARG1+ | NEK4 | IFN Beta Up | TSC1 | PI3K/AKT Signaling |
| | infiltrating macrophages | | | | |
| | STA | | | | |
| HIF1A | CCR2+ARG1+ | NFE2L3 | IFN Beta Up | AKT2 | PI3K/AKT Signaling |
| | infiltrating macrophages | | | | |
| | STA | | | | |
| HILPDA | CCR2+ARG1+ | NKTR | IFN Beta Up | AKT3 | PI3K/AKT Signaling |
| | infiltrating macrophages | | | | |
| | STA | | | | |
| HNRNPA0 | CCR2+ARG1+ | NMI | IFN Beta Up | BCL2L1 | PI3K/AKT Signaling |
| | infiltrating macrophages | | | | |
| | STA | | | | |
| HSP90AA1 | CCR2+ARG1+ | NOTCH1 | IFN Beta Up | CCND1 | PI3K/AKT Signaling |
| | infiltrating macrophages | | | | |
| | STA | | | | |
| IFRD1 | CCR2+ARG1+ | NR3C1 | IFN Beta Up | CDC37 | PI3K/AKT Signaling |
| | infiltrating macrophages | | | | |
| | STA | | | | |
| IMPDH2 | CCR2+ARG1+ | NR4A3 | IFN Beta Up | CDKN1B | PI3K/AKT Signaling |
| | infiltrating macrophages | | | | |
| | STA | | | | |
| ITGAV | CCR2+ARG1+ | NUB1 | IFN Beta Up | EIF4E | PI3K/AKT Signaling |
| | infiltrating macrophages | | | | |
| | STA | | | | |
| LGALS3 | CCR2+ARG1+ | NUPR1 | IFN Beta Up | GRB2 | PI3K/AKT Signaling |
| | infiltrating macrophages | | | | |
| | STA | | | | |
| M6PR | CCR2+ARG1+ | OAS1 | IFN Beta Up | GYS1 | PI3K/AKT Signaling |
| | infiltrating macrophages | | | | |
| | STA | | | | |
| MAP4K4 | CCR2+ARG1+ | OAS2 | IFN Beta Up | HRAS | PI3K/AKT Signaling |
| | infiltrating macrophages | | | | |
| | STA | | | | |
| MCL1 | CCR2+ARG1+ | OAS3 | IFN Beta Up | HSP90AA1 | PI3K/AKT Signaling |
| | infiltrating macrophages | | | | |
| | STA | | | | |
| MDM2 | CCR2+ARG1+ | PATJ | IFN Beta Up | HSP90AB1 | PI3K/AKT Signaling |
| | infiltrating macrophages | | | | |
| | STA | | | | |
| MS4A7 | CCR2+ARG1+ | PAX5 | IFN Beta Up | IKBKB | PI3K/AKT Signaling |
| | infiltrating macrophages | | | | |
| | STA | | | | |
| NAA50 | CCR2+ARG1+ | PAX8 | IFN Beta Up | IKBKE | PI3K/AKT Signaling |
| | infiltrating macrophages | | | | |
| | STA | | | | |
| NDUFAB1 | CCR2+ARG1+ | PDE4B | IFN Beta Up | ITGA3 | PI3K/AKT Signaling |
| | infiltrating macrophages | | | | |
| | STA | | | | |
| NDUFS6 | CCR2+ARG1+ | PDGFB | IFN Beta Up | ITGA4 | PI3K/AKT Signaling |
| | infiltrating macrophages | | | | |
| | STA | | | | |
| NFATC1 | CCR2+ARG1+ | PDGFRL | IFN Beta Up | ITGB1 | PI3K/AKT Signaling |
| | infiltrating macrophages | | | | |
| | STA | | | | |
| NFKBIA | CCR2+ARG1+ | PFKFB3 | IFN Beta Up | JAK1 | PI3K/AKT Signaling |
| | infiltrating macrophages | | | | |
| | STA | | | | |
| NHP2 | CCR2+ARG1+ | PFKP | IFN Beta Up | JAK2 | PI3K/AKT Signaling |
| | infiltrating macrophages | | | | |
| | STA | | | | |
| NOP10 | CCR2+ARG1+ | PIM2 | IFN Beta Up | KRAS | PI3K/AKT Signaling |
| | infiltrating macrophages | | | | |
| | STA | | | | |
| NOP58 | CCR2+ARG1+ | PKD2 | IFN Beta Up | MAP2K1 | PI3K/AKT Signaling |
| | infiltrating macrophages | | | | |
| | STA | | | | |
| NPM3 | CCR2+ARG1+ | PLEK | IFN Beta Up | MAP2K2 | PI3K/AKT Signaling |
| | infiltrating macrophages | | | | |
| | STA | | | | |
| NRP2 | CCR2+ARG1+ | PLSCR1 | IFN Beta Up | MAPK1 | PI3K/AKT Signaling |
| | infiltrating macrophages | | | | |
| | STA | | | | |
| ODC1 | CCR2+ARG1+ | PMAIP1 | IFN Beta Up | MAPK3 | PI3K/AKT Signaling |
| | infiltrating macrophages | | | | |
| | STA | | | | |
| OSBPL8 | CCR2+ARG1+ | PML | IFN Beta Up | MDM2 | PI3K/AKT Signaling |
| | infiltrating macrophages | | | | |
| | STA | | | | |
| PPA1 | CCR2+ARG1+ | MED1 | IFN Beta Up | MTOR | PI3K/AKT Signaling |
| | infiltrating macrophages | | | | |
| | STA | | | | |
| PSMB5 | CCR2+ARG1+ | PPP2R2A | IFN Beta Up | NFKB2 | PI3K/AKT Signaling |
| | infiltrating macrophages | | | | |
| | STA | | | | |
| PSMB6 | CCR2+ARG1+ | PRKAG1 | IFN Beta Up | NFKBIA | PI3K/AKT Signaling |
| | infiltrating macrophages | | | | |
| | STA | | | | |
| PTBP3 | CCR2+ARG1+ | EIF2AK2 | IFN Beta Up | NFKBIE | PI3K/AKT Signaling |
| | infiltrating macrophages | | | | |
| | STA | | | | |
| PTMA | CCR2+ARG1+ | PRKRA | IFN Beta Up | NRAS | PI3K/AKT Signaling |
| | infiltrating macrophages | | | | |
| | STA | | | | |
| RAN | CCR2+ARG1+ | PRKX | IFN Beta Up | PDPK1 | PI3K/AKT Signaling |
| | infiltrating macrophages | | | | |
| | STA | | | | |
| RNF19B | CCR2+ARG1+ | PSMB8 | IFN Beta Up | PIK3CB | PI3K/AKT Signaling |
| | infiltrating macrophages | | | | |
| | STA | | | | |
| RPL22 | CCR2+ARG1+ | PSMB9 | IFN Beta Up | PIK3CD | PI3K/AKT Signaling |
| | infiltrating macrophages | | | | |
| | STA | | | | |
| RPL27 | CCR2+ARG1+ | PTCH1 | IFN Beta Up | PIK3CG | PI3K/AKT Signaling |
| | infiltrating macrophages | | | | |
| | STA | | | | |
| RPLP1 | CCR2+ARG1+ | PTGER2 | IFN Beta Up | RHEB | PI3K/AKT Signaling |
| | infiltrating macrophages | | | | |
| | STA | | | | |
| RPS15A | CCR2+ARG1+ | RALB | IFN Beta Up | RPS6KB1 | PI3K/AKT Signaling |
| | infiltrating macrophages | | | | |
| | STA | | | | |
| RPS2 | CCR2+ARG1+ | RASGRP1 | IFN Beta Up | RRAS | PI3K/AKT Signaling |
| | infiltrating macrophages | | | | |
| | STA | | | | |
| SET | CCR2+ARG1+ | RBBP6 | IFN Beta Up | SHC1 | PI3K/AKT Signaling |
| | infiltrating macrophages | | | | |
| | STA | | | | |
| SRSF3 | CCR2+ARG1+ | RBCK1 | IFN Beta Up | SOS2 | PI3K/AKT Signaling |
| | infiltrating macrophages | | | | |
| | STA | | | | |
| SRSF7 | CCR2+ARG1+ | RERE | IFN Beta Up | YWHAE | PI3K/AKT Signaling |
| | infiltrating macrophages | | | | |
| | STA | | | | |
| TIMM13 | CCR2+ARG1+ | RGS1 | IFN Beta Up | YWHAH | PI3K/AKT Signaling |
| | infiltrating macrophages | | | | |
| | STA | | | | |
| TNFAIP3 | CCR2+ARG1+ | RGS6 | IFN Beta Up | YWHAZ | PI3K/AKT Signaling |
| | infiltrating macrophages | | | | |
| | STA | | | | |
| TPM1 | CCR2+ARG1+ | RIN1 | IFN Beta Up | IRF4 | Plasma Cells |
| | infiltrating macrophages | | | | |
| | STA | | | | |
| UFM1 | CCR2+ARG1+ | RIPK1 | IFN Beta Up | PRDM1 | Plasma Cells |
| | infiltrating macrophages | | | | |
| | STA | | | | |
| UQCRQ | CCR2+ARG1+ | RIPK3 | IFN Beta Up | XBP1 | Plasma Cells |
| | infiltrating macrophages | | | | |
| | STA | | | | |
| YWHAZ | CCR2+ARG1+ | RNF114 | IFN Beta Up | IGHD | Plasma Cells |
| | infiltrating macrophages | | | | |
| | STA | | | | |
| ACTR3 | CCR2+IL1B+ infiltrating | RPS6KA5 | IFN Beta Up | IGHM | Plasma Cells |
| | macrophages STA | | | | |
| AIF1 | CCR2+IL1B+ infiltrating | RPS9 | IFN Beta Up | CD38 | Plasma Cells |
| | macrophages STA | | | | |
| ANKRD11 | CCR2+IL1B+ infiltrating | RRBP1 | IFN Beta Up | SLAMF7 | Plasma Cells |
| | macrophages STA | | | | |
| ARHGDIB | CCR2+IL1B+ infiltrating | RTP4 | IFN Beta Up | IGHG1 | Plasma Cells |
| | macrophages STA | | | | |
| ARPC4 | CCR2+IL1B+ infiltrating | SAT1 | IFN Beta Up | IGHV1-2 | Plasma Cells |
| | macrophages STA | | | | |
| BIN2 | CCR2+IL1B+ infiltrating | SCARB2 | IFN Beta Up | IGHV1-46 | Plasma Cells |
| | macrophages STA | | | | |
| BIRC3 | CCR2+IL1B+ infiltrating | SDS | IFN Beta Up | IGHV3-21 | Plasma Cells |
| | macrophages STA | | | | |
| CAPZA2 | CCR2+IL1B+ infiltrating | SELL | IFN Beta Up | IGHV3-23 | Plasma Cells |
| | macrophages STA | | | | |
| CCND3 | CCR2+IL1B+ infiltrating | SERPIND1 | IFN Beta Up | IGHV4-31 | Plasma Cells |
| | macrophages STA | | | | |
| CCR1 | CCR2+IL1B+ infiltrating | SERPING1 | IFN Beta Up | IGHV4-34 | Plasma Cells |
| | macrophages STA | | | | |
| CD53 | CCR2+IL1B+ infiltrating | SFTPB | IFN Beta Up | IGK | Plasma Cells |
| | macrophages STA | | | | |
| CFP | CCR2+IL1B+ infiltrating | SIDT2 | IFN Beta Up | IGKC | Plasma Cells |
| | macrophages STA | | | | |
| CORO1A | CCR2+IL1B+ infiltrating | SIT1 | IFN Beta Up | IGKVID-8 | Plasma Cells |
| | macrophages STA | | | | |
| CSF2RA | CCR2+IL1B+ infiltrating | SLAMF1 | IFN Beta Up | IGKV4-1 | Plasma Cells |
| | macrophages STA | | | | |
| CSF2RB | CCR2+IL1B+ infiltrating | SMO | IFN Beta Up | IGLC1 | Plasma Cells |
| | macrophages STA | | | | |
| CTSC | CCR2+IL1B+ infiltrating | SNX2 | IFN Beta Up | IGLJ3 | Plasma Cells |
| | macrophages STA | | | | |
| CTSH | CCR2+IL1B+ infiltrating | SOCS1 | IFN Beta Up | IGLV1-40 | Plasma Cells |
| | macrophages STA | | | | |
| CXCL3 | CCR2+IL1B+ infiltrating | SOS1 | IFN Beta Up | IGLV1-44 | Plasma Cells |
| | macrophages STA | | | | |
| CYTIP | CCR2+IL1B+ infiltrating | SP100 | IFN Beta Up | IGLV2-14 | Plasma Cells |
| | macrophages STA | | | | |
| EMILIN2 | CCR2+IL1B+ infiltrating | SP110 | IFN Beta Up | IGLV3-10 | Plasma Cells |
| | macrophages STA | | | | |
| ENO1 | CCR2+IL1B+ infiltrating | SP140 | IFN Beta Up | IGLV3-19 | Plasma Cells |
| | macrophages STA | | | | |
| FAM107B | CCR2+IL1B+ infiltrating | SPIB | IFN Beta Up | IGLV3-25 | Plasma Cells |
| | macrophages STA | | | | |
| FAM96A | CCR2+IL1B+ infiltrating | SPTA1 | IFN Beta Up | IGLVI-70 | Plasma Cells |
| | macrophages STA | | | | |
| FGR | CCR2+IL1B+ infiltrating | SPTLC2 | IFN Beta Up | LIME1 | Plasma Cells |
| | macrophages STA | | | | |
| FYB | CCR2+IL1B+ infiltrating | SRRM2 | IFN Beta Up | MZB1 | Plasma Cells |
| | macrophages STA | | | | |
| GLIPR2 | CCR2+IL1B+ infiltrating | SSB | IFN Beta Up | RAD51AP1 | Plasma Cells |
| | macrophages STA | | | | |
| GM2A | CCR2+IL1B+ infiltrating | ST3GAL5 | IFN Beta Up | C19orf10 | Plasma Cells |
| | macrophages STA | | | | |
| GMFG | CCR2+IL1B+ infiltrating | STAP1 | IFN Beta Up | IGH | Plasma Cells |
| | macrophages STA | | | | |
| GPR132 | CCR2+IL1B+ infiltrating | STAT1 | IFN Beta Up | IGHMBP2 | Plasma Cells |
| | macrophages STA | | | | |
| H2AFY | CCR2+IL1B+ infiltrating | STAT2 | IFN Beta Up | IGH4-34 | Plasma Cells |
| | macrophages STA | | | | |
| HCLS1 | CCR2+IL1B+ infiltrating | STOML2 | IFN Beta Up | IGLL1 | Plasma Cells |
| | macrophages STA | | | | |
| HLA-DMB | CCR2+IL1B+ infiltrating | STX11 | IFN Beta Up | IGLV@ | Plasma Cells |
| | macrophages STA | | | | |
| HPCAL1 | CCR2+IL1B+ infiltrating | SUPT3H | IFN Beta Up | IGLV3-1 | Plasma Cells |
| | macrophages STA | | | | |
| IFI16 | CCR2+IL1B+ infiltrating | TANK | IFN Beta Up | IGLV4-3 | Plasma Cells |
| | macrophages STA | | | | |
| IFNAR2 | CCR2+IL1B+ infiltrating | TAP1 | IFN Beta Up | IGH4-28 | Plasma Cells |
| | macrophages STA | | | | |
| IFNGR1 | CCR2+IL1B+ infiltrating | TAP2 | IFN Beta Up | IGLV4-60 | Plasma Cells |
| | macrophages STA | | | | |
| IGSF6 | CCR2+IL1B+ infiltrating | TAPBP | IFN Beta Up | IGLV5-45 | Plasma Cells |
| | macrophages STA | | | | |
| IL17RA | CCR2+IL1B+ infiltrating | TARBP1 | IFN Beta Up | IGLV6-57 | Plasma Cells |
| | macrophages STA | | | | |
| ITGB2 | CCR2+IL1B+ infiltrating | TBX21 | IFN Beta Up | PRDM1 | Plasma Cells |
| | macrophages STA | | | | |
| JUN | CCR2+IL1B+ infiltrating | TCN2 | IFN Beta Up | THEMIS2 | Plasma Cells |
| | macrophages STA | | | | |
| LGALS3 | CCR2+IL1B+ infiltrating | TFDP2 | IFN Beta Up | SDC1 | Plasma Cells |
| | macrophages STA | | | | |
| LILRB3 | CCR2+IL1B+ infiltrating | TFF1 | IFN Beta Up | IGHV3-47 | Plasma Cells |
| | macrophages STA | | | | |
| LMAN2 | CCR2+IL1B+ infiltrating | TGM1 | IFN Beta Up | IGHV4-28 | Plasma Cells |
| | macrophages STA | | | | |
| LY6E | CCR2+IL1B+ infiltrating | THY1 | IFN Beta Up | IGHV5-78 | Plasma Cells |
| | macrophages STA | | | | |
| LYN | CCR2+IL1B+ infiltrating | TLR1 | IFN Beta Up | IGLL3P | Plasma Cells |
| | macrophages STA | | | | |
| MYD88 | CCR2+IL1B+ infiltrating | TLR3 | IFN Beta Up | IGLV7-43 | Plasma Cells |
| | macrophages STA | | | | |
| MYL12A | CCR2+IL1B+ infiltrating | TLR7 | IFN Beta Up | BRCA1 | Pro Cell Cycle |
| | macrophages STA | | | | |
| NCF2 | CCR2+IL1B+ infiltrating | TNFAIP2 | IFN Beta Up | MCM2 | Pro Cell Cycle |
| | macrophages STA | | | | |
| NFE2L2 | CCR2+IL1B+ infiltrating | TNFRSF11A | IFN Beta Up | NDC80 | Pro Cell Cycle |
| | macrophages STA | | | | |
| NFKBIA | CCR2+IL1B+ infiltrating | TNFRSF6 | IFN Beta Up | PTTG1 | Pro Cell Cycle |
| | macrophages STA | | | | |
| NMI | CCR2+IL1B+ infiltrating | TNFSF10 | IFN Beta Up | E2F3 | Pro Cell Cycle |
| | macrophages STA | | | | |
| NRROS | CCR2+IL1B+ infiltrating | TNFSF6 | IFN Beta Up | ASPM | Pro Cell Cycle |
| | macrophages STA | | | | |
| PICALM | CCR2+IL1B+ infiltrating | TOR1B | IFN Beta Up | AURKA | Pro Cell Cycle |
| | macrophages STA | | | | |
| PLA2G7 | CCR2+IL1B+ infiltrating | TRA2B | IFN Beta Up | CCNB2 | Pro Cell Cycle |
| | macrophages STA | | | | |
| PLEK | CCR2+IL1B+ infiltrating | TRIM21 | IFN Beta Up | CCNE1 | Pro Cell Cycle |
| | macrophages STA | | | | |
| PPP1R15A | CCR2+IL1B+ infiltrating | TRIM22 | IFN Beta Up | CDC20 | Pro Cell Cycle |
| | macrophages STA | | | | |
| PPP4C | CCR2+IL1B+ infiltrating | TRIM26 | IFN Beta Up | CENPM | Pro Cell Cycle |
| | macrophages STA | | | | |
| PSMB10 | CCR2+IL1B+ infiltrating | TRIM38 | IFN Beta Up | CEP55 | Pro Cell Cycle |
| | macrophages STA | | | | |
| PSMB8 | CCR2+IL1B+ infiltrating | TSPAN15 | IFN Beta Up | GINS2 | Pro Cell Cycle |
| | macrophages STA | | | | |
| PSMB9 | CCR2+IL1B+ infiltrating | TXK | IFN Beta Up | MCM10 | Pro Cell Cycle |
| | macrophages STA | | | | |
| PSME1 | CCR2+IL1B+ infiltrating | UBE2L6 | IFN Beta Up | CCNB1 | Pro Cell Cycle |
| | macrophages STA | | | | |
| PTPN6 | CCR2+IL1B+ infiltrating | UBE2S | IFN Beta Up | TYMS | Pro Cell Cycle |
| | macrophages STA | | | | |
| PTPRC | CCR2+IL1B+ infiltrating | UBE3A | IFN Beta Up | NCAPG | Pro Cell Cycle |
| | macrophages STA | | | | |
| PYCARD | CCR2+IL1B+ infiltrating | UBQLN2 | IFN Beta Up | AURKB | Pro Cell Cycle |
| | macrophages STA | | | | |
| RAC2 | CCR2+IL1B+ infiltrating | UNC93B1 | IFN Beta Up | MKI67 | Pro Cell Cycle |
| | macrophages STA | | | | |
| RBM39 | CCR2+IL1B+ infiltrating | USP15 | IFN Beta Up | PTPN6 | Production of NO and |
| | macrophages STA | | | | ROS in Macrophages |
| RUNX3 | CCR2+IL1B+ infiltrating | USP18 | IFN Beta Up | RAP1A | Production of NO and |
| | macrophages STA | | | | ROS in Macrophages |
| SAMHD1 | CCR2+IL1B+ infiltrating | USP25 | IFN Beta Up | RAP1B | Production of NO and |
| | macrophages STA | | | | ROS in Macrophages |
| SERP1 | CCR2+IL1B+ infiltrating | USPL1 | IFN Beta Up | SPI1 | Production of NO and |
| | macrophages STA | | | | ROS in Macrophages |
| SF3B1 | CCR2+IL1B+ infiltrating | UVRAG | IFN Beta Up | ARG2 | Production of NO and |
| | macrophages STA | | | | ROS in Macrophages |
| SLC15A3 | CCR2+IL1B+ infiltrating | VAMP5 | IFN Beta Up | PPM1L | Production of NO and |
| | macrophages STA | | | | ROS in Macrophages |
| SLFN5 | CCR2+IL1B+ infiltrating | WARS | IFN Beta Up | PPP1CB | Production of NO and |
| | macrophages STA | | | | ROS in Macrophages |
| SPCS2 | CCR2+IL1B+ infiltrating | WIPF1 | IFN Beta Up | PPP1R7 | Production of NO and |
| | macrophages STA | | | | ROS in Macrophages |
| SRSF3 | CCR2+IL1B+ infiltrating | WT1 | IFN Beta Up | PPP1R10 | Production of NO and |
| | macrophages STA | | | | ROS in Macrophages |
| TKT | CCR2+IL1B+ infiltrating | XAF1 | IFN Beta Up | PPP1R12A | Production of NO and |
| | macrophages STA | | | | ROS in Macrophages |
| TLR2 | CCR2+IL1B+ infiltrating | ZNF107 | IFN Beta Up | PPP1R14A | Production of NO and |
| | macrophages STA | | | | ROS in Macrophages |
| TMBIM6 | CCR2+IL1B+ infiltrating | EIF2AK2 | IFN Core Signature | PPP1R14B | Production of NO and |
| | macrophages STA | | | | ROS in Macrophages |
| TMED2 | CCR2+IL1B+ infiltrating | GBP1 | IFN Core Signature | PPP1R3D | Production of NO and |
| | macrophages STA | | | | ROS in Macrophages |
| TOR1AIP1 | CCR2+IL1B+ infiltrating | HERC5 | IFN Core Signature | PPP2CB | Production of NO and |
| | macrophages STA | | | | ROS in Macrophages |
| TPD52 | CCR2+IL1B+ infiltrating | HERC6 | IFN Core Signature | PPP2R2A | Production of NO and |
| | macrophages STA | | | | ROS in Macrophages |
| TPM4 | CCR2+IL1B+ infiltrating | IFI27 | IFN Core Signature | PPP2R5C | Production of NO and |
| | macrophages STA | | | | ROS in Macrophages |
| TRAM1 | CCR2+IL1B+ infiltrating | IFI35 | IFN Core Signature | PPP2R5E | Production of NO and |
| | macrophages STA | | | | ROS in Macrophages |
| TRPS1 | CCR2+IL1B+ infiltrating | IFI44 | IFN Core Signature | AKT2 | Production of NO and |
| | macrophages STA | | | | ROS in Macrophages |
| UBC | CCR2+IL1B+ infiltrating | IFI44L | IFN Core Signature | AKT3 | Production of NO and |
| | macrophages STA | | | | ROS in Macrophages |
| UBE2D3 | CCR2+IL1B+ infiltrating | IFI6 | IFN Core Signature | APOB | Production of NO and |
| | macrophages STA | | | | ROS in Macrophages |
| UBE2N | CCR2+IL1B+ infiltrating | IFIT1 | IFN Core Signature | APOL1 | Production of NO and |
| | macrophages STA | | | | ROS in Macrophages |
| UCP2 | CCR2+IL1B+ infiltrating | IFIT3 | IFN Core Signature | ATM | Production of NO and |
| | macrophages STA | | | | ROS in Macrophages |
| VASP | CCR2+IL1B+ infiltrating | IFIT5 | IFN Core Signature | CLU | Production of NO and |
| | macrophages STA | | | | ROS in Macrophages |
| VMP1 | CCR2+IL1B+ infiltrating | IFITM1 | IFN Core Signature | CREBBP | Production of NO and |
| | macrophages STA | | | | ROS in Macrophages |
| ZBP1 | CCR2+IL1B+ infiltrating | IFITM3 | IFN Core Signature | CYBA | Production of NO and |
| | macrophages STA | | | | ROS in Macrophages |
| ZEB2 | CCR2+IL1B+ infiltrating | ISG20 | IFN Core Signature | CYBB | Production of NO and |
| | macrophages STA | | | | ROS in Macrophages |
| ZYX | CCR2+IL1B+ infiltrating | MX1 | IFN Core Signature | DIRAS3 | Production of NO and |
| | macrophages STA | | | | ROS in Macrophages |
| RUNX3 | CD8 T Cells | MX2 | IFN Core Signature | FGFR1 | Production of NO and |
| | | | | | ROS in Macrophages |
| CD8A | CD8 T Cells | RSAD2 | IFN Core Signature | FNBP1 | Production of NO and |
| | | | | | ROS in Macrophages |
| CD8B | CD8 T Cells | SP100 | IFN Core Signature | FRS2 | Production of NO and |
| | | | | | ROS in Macrophages |
| CD3D | CD8 T Cells | GBP2 | IFN Core Signature | GAB1 | Production of NO and |
| | | | | | ROS in Macrophages |
| TRAV6 | CD8 T Cells | GBP4 | IFN Core Signature | GRB2 | Production of NO and |
| | | | | | ROS in Macrophages |
| EOMES | CD8 T Cells | IFIT2 | IFN Core Signature | IFNGR1 | Production of NO and |
| | | | | | ROS in Macrophages |
| CST7 | CD8 T Cells | IFITM2 | IFN Core Signature | IKBKB | Production of NO and |
| | | | | | ROS in Macrophages |
| IL16 | CD8 T Cells | SAMD9 | IFN Core Signature | IKBKE | Production of NO and |
| | | | | | ROS in Macrophages |
| JAKMIP1 | CD8 T Cells | SAMD9L | IFN Core Signature | IRF1 | Production of NO and |
| | | | | | ROS in Macrophages |
| KLRB1 | CD8 T Cells | SP110 | IFN Core Signature | IRF8 | Production of NO and |
| | | | | | ROS in Macrophages |
| CD300A | CD8 T Cells | ISG15 | IFN Core Signature | IRS1 | Production of NO and |
| | | | | | ROS in Macrophages |
| NKG7 | CD8 T Cells | OAS1 | IFN Core Signature | JAK1 | Production of NO and |
| | | | | | ROS in Macrophages |
| BATF3 | CD8 T Cells | OAS2 | IFN Core Signature | JAK2 | Production of NO and |
| | | | | | ROS in Macrophages |
| IL10 | CD8 T Cells | OAS3 | IFN Core Signature | JUN | Production of NO and |
| | | | | | ROS in Macrophages |
| BATF2 | CD8 T Cells | OASL | IFN Core Signature | MAP2K1 | Production of NO and |
| | | | | | ROS in Macrophages |
| CD226 | CD8 T Cells | AQP9 | IFN-activated | MAP2K4 | Production of NO and |
| | | | monocytes | | ROS in Macrophages |
| CD247 | CD8 T Cells | C15orf48 | IFN-activated | MAP3K1 | Production of NO and |
| | | | monocytes | | ROS in Macrophages |
| CD160 | CD8 T Cells | CD52 | IFN-activated | MAP3K2 | Production of NO and |
| | | | monocytes | | ROS in Macrophages |
| CD244 | CD8 T Cells | FN1 | IFN-activated | MAP3K3 | Production of NO and |
| | | | monocytes | | ROS in Macrophages |
| IL23R | CD8 T Cells | IFI44L | IFN-activated | MAP3K4 | Production of NO and |
| | | | monocytes | | ROS in Macrophages |
| CCL5 | CD8 T Cells | IFI6 | IFN-activated | MAP3K5 | Production of NO and |
| | | | monocytes | | ROS in Macrophages |
| CTSW | CD8 T Cells | IGHM | IFN-activated | MAP3K7 | Production of NO and |
| | | | monocytes | | ROS in Macrophages |
| GZMA | CD8 T Cells | ISG15 | IFN-activated | MAP3K12 | Production of NO and |
| | | | monocytes | | ROS in Macrophages |
| GZMH | CD8 T Cells | ITGB7 | IFN-activated | MAPK1 | Production of NO and |
| | | | monocytes | | ROS in Macrophages |
| PRF1 | CD8 T Cells | LY6E | IFN-activated | MAPK3 | Production of NO and |
| | | | monocytes | | ROS in Macrophages |
| FGL2 | CD8 T Cells | SLAMF7 | IFN-activated | MAPK8 | Production of NO and |
| | | | monocytes | | ROS in Macrophages |
| SRGN | CD8 T Cells | WARS | IFN-activated | MAPK9 | Production of NO and |
| | | | monocytes | | ROS in Macrophages |
| CD96 | CD8 T Cells | AKT2 | IL-8 Signaling | MAPK14 | Production of NO and |
| | | | | | ROS in Macrophages |
| GZMB | CD8 T Cells | AKT3 | IL-8 Signaling | NCF2 | Production of NO and |
| | | | | | ROS in Macrophages |
| GZMK | CD8 T Cells | ANGPT1 | IL-8 Signaling | NCF4 | Production of NO and |
| | | | | | ROS in Macrophages |
| GNLY | CD8 T Cells | ARRB2 | IL-8 Signaling | NFKB2 | Production of NO and |
| | | | | | ROS in Macrophages |
| GZMM | CD8 T Cells | ATM | IL-8 Signaling | NFKBIA | Production of NO and |
| | | | | | ROS in Macrophages |
| KIR2DL1 | CD8 T Cells | BAX | IL-8 Signaling | NFKBIE | Production of NO and |
| | | | | | ROS in Macrophages |
| KIR2DL2 | CD8 T Cells | BCL2L1 | IL-8 Signaling | NGFR | Production of NO and |
| | | | | | ROS in Macrophages |
| KIR2DL3 | CD8 T Cells | BRAF | IL-8 Signaling | PCYOX1 | Production of NO and |
| | | | | | ROS in Macrophages |
| KIR2DL4 | CD8 T Cells | CCND1 | IL-8 Signaling | PIK3C3 | Production of NO and |
| | | | | | ROS in Macrophages |
| KIR2DL5A | CD8 T Cells | CCND2 | IL-8 Signaling | PIK3C2A | Production of NO and |
| | | | | | ROS in Macrophages |
| KIR2DS1 | CD8 T Cells | CCND3 | IL-8 Signaling | PIK3C2B | Production of NO and |
| | | | | | ROS in Macrophages |
| KIR2DS2 | CD8 T Cells | CSTB | IL-8 Signaling | PIK3CB | Production of NO and |
| | | | | | ROS in Macrophages |
| KIR2DS3 | CD8 T Cells | CXCL8 | IL-8 Signaling | PIK3CD | Production of NO and |
| | | | | | ROS in Macrophages |
| KIR2DS5 | CD8 T Cells | CYBB | IL-8 Signaling | PIK3CG | Production of NO and |
| | | | | | ROS in Macrophages |
| KIR3DL1 | CD8 T Cells | DIRAS3 | IL-8 Signaling | PIK3R1 | Production of NO and |
| | | | | | ROS in Macrophages |
| KIR3DL3 | CD8 T Cells | EGFR | IL-8 Signaling | PLCG1 | Production of NO and |
| | | | | | ROS in Macrophages |
| KIR3DS1 | CD8 T Cells | FGFR1 | IL-8 Signaling | PLCG2 | Production of NO and |
| | | | | | ROS in Macrophages |
| KIR3DX1 | CD8 T Cells | FLT1 | IL-8 Signaling | PPARA | Production of NO and |
| | | | | | ROS in Macrophages |
| KLRC3 | CD8 T Cells | FNBP1 | IL-8 Signaling | PRKCA | Production of NO and |
| | | | | | ROS in Macrophages |
| KLRC4 | CD8 T Cells | FRS2 | IL-8 Signaling | PRKCB | Production of NO and |
| | | | | | ROS in Macrophages |
| KLRD1 | CD8 T Cells | GAB1 | IL-8 Signaling | PRKCD | Production of NO and |
| | | | | | ROS in Macrophages |
| KLRG1 | CD8 T Cells | GNA13 | IL-8 Signaling | PRKCI | Production of NO and |
| | | | | | ROS in Macrophages |
| UNC13D | CD8 T Cells | GNAS | IL-8 Signaling | PRKD1 | Production of NO and |
| | | | | | ROS in Macrophages |
| C1QA | Complement | GNB1 | IL-8 Signaling | PRKD3 | Production of NO and |
| | | | | | ROS in Macrophages |
| C1QB | Complement | GNB4 | IL-8 Signaling | PTPN11 | Production of NO and |
| | | | | | ROS in Macrophages |
| C1QC | Complement | GNB5 | IL-8 Signaling | RAC1 | Production of NO and |
| | | | | | ROS in Macrophages |
| C2 | Complement | GNG2 | IL-8 Signaling | RHOA | Production of NO and |
| | | | | | ROS in Macrophages |
| C3 | Complement | GNG5 | IL-8 Signaling | RHOB | Production of NO and |
| | | | | | ROS in Macrophages |
| C4BPA | Complement | GNG11 | IL-8 Signaling | RHOH | Production of NO and |
| | | | | | ROS in Macrophages |
| C4BPB | Complement | GNG12 | IL-8 Signaling | RHOJ | Production of NO and |
| | | | | | ROS in Macrophages |
| C5 | Complement | GRB2 | IL-8 Signaling | RHOQ | Production of NO and |
| | | | | | ROS in Macrophages |
| C7 | Complement | HRAS | IL-8 Signaling | RHOT2 | Production of NO and |
| | | | | | ROS in Macrophages |
| C6 | Complement | ICAM1 | IL-8 Signaling | RHOU | Production of NO and |
| | | | | | ROS in Macrophages |
| C8a | Complement | IKBKB | IL-8 Signaling | RND3 | Production of NO and |
| | | | | | ROS in Macrophages |
| C9 | Complement | IKBKE | IL-8 Signaling | SERPINA1 | Production of NO and |
| | | | | | ROS in Macrophages |
| CFP | Complement | IQGAP1 | IL-8 Signaling | SIRPA | Production of NO and |
| | | | | | ROS in Macrophages |
| ABCA1 | CX3CR1+ lining | IRAK3 | IL-8 Signaling | STAT1 | Production of NO and |
| | macrophages STA | | | | ROS in Macrophages |
| AHNAK2 | CX3CR1+ lining | IRS1 | IL-8 Signaling | TLR2 | Production of NO and |
| | macrophages STA | | | | ROS in Macrophages |
| APP | CX3CR1+ lining | ITGAV | IL-8 Signaling | TLR4 | Production of NO and |
| | macrophages STA | | | | ROS in Macrophages |
| ARL11 | CX3CR1+ lining | ITGAX | IL-8 Signaling | TNFRSF11B | Production of NO and |
| | macrophages STA | | | | ROS in Macrophages |
| B2M | CX3CR1+ lining | ITGB2 | IL-8 Signaling | TNFRSF1B | Production of NO and |
| | macrophages STA | | | | ROS in Macrophages |
| BCL2A1 | CX3CR1+ lining | ITGB5 | IL-8 Signaling | APOL1 | Quiescent macrophages |
| | macrophages STA | | | | |
| BLNK | CX3CR1+ lining | JUN | IL-8 Signaling | CROCCP2 | Quiescent macrophages |
| | macrophages STA | | | | |
| C1QA | CX3CR1+ lining | KRAS | IL-8 Signaling | HLA-DQB2 | Quiescent macrophages |
| | macrophages STA | | | | |
| C1QB | CX3CR1+ lining | MAP2K1 | IL-8 Signaling | IFI27 | Quiescent macrophages |
| | macrophages STA | | | | |
| C1QC | CX3CR1+ lining | MAP2K2 | IL-8 Signaling | ACTR2 | Rac Signaling |
| | macrophages STA | | | | |
| CD37 | CX3CR1+ lining | MAP2K4 | IL-8 Signaling | ARPC4 | Rac Signaling |
| | macrophages STA | | | | |
| CD40 | CX3CR1+ lining | MAP4K4 | IL-8 Signaling | ARPC5 | Rac Signaling |
| | macrophages STA | | | | |
| CD83 | CX3CR1+ lining | MAPK1 | IL-8 Signaling | CD44 | Rac Signaling |
| | macrophages STA | | | | |
| CD84 | CX3CR1+ lining | MAPK3 | IL-8 Signaling | CDC42 | Rac Signaling |
| | macrophages STA | | | | |
| CHD9 | CX3CR1+ lining | MAPK8 | IL-8 Signaling | CFL1 | Rac Signaling |
| | macrophages STA | | | | |
| CLEC12A | CX3CR1+ lining | MAPK9 | IL-8 Signaling | CYBB | Rac Signaling |
| | macrophages STA | | | | |
| CLIC4 | CX3CR1+ lining | MTOR | IL-8 Signaling | FGFR1 | Rac Signaling |
| | macrophages STA | | | | |
| COMT | CX3CR1+ lining | NAPEPLD | IL-8 Signaling | GRB2 | Rac Signaling |
| | macrophages STA | | | | |
| CSF1R | CX3CR1+ lining | NCF2 | IL-8 Signaling | IQGAP1 | Rac Signaling |
| | macrophages STA | | | | |
| CSPG4 | CX3CR1+ lining | NRAS | IL-8 Signaling | IQGAP2 | Rac Signaling |
| | macrophages STA | | | | |
| CTSB | CX3CR1+ lining | PAK2 | IL-8 Signaling | KRAS | Rac Signaling |
| | macrophages STA | | | | |
| CTSS | CX3CR1+ lining | PDGFC | IL-8 Signaling | MAP3K1 | Rac Signaling |
| | macrophages STA | | | | |
| DDX3X | CX3CR1+ lining | PIK3C3 | IL-8 Signaling | MAPK1 | Rac Signaling |
| | macrophages STA | | | | |
| DSTN | CX3CR1+ lining | PIK3C2A | IL-8 Signaling | MAPK3 | Rac Signaling |
| | macrophages STA | | | | |
| DUSP3 | CX3CR1+ lining | PIK3C2B | IL-8 Signaling | NCF2 | Rac Signaling |
| | macrophages STA | | | | |
| ENPP1 | CX3CR1+ lining | PIK3CB | IL-8 Signaling | NFKB2 | Rac Signaling |
| | macrophages STA | | | | |
| ENTPD1 | CX3CR1+ lining | PIK3CD | IL-8 Signaling | PAK1 | Rac Signaling |
| | macrophages STA | | | | |
| F11R | CX3CR1+ lining | PIK3CG | IL-8 Signaling | PAK2 | Rac Signaling |
| | macrophages STA | | | | |
| FERMT2 | CX3CR1+ lining | PIK3R1 | IL-8 Signaling | PIK3C2A | Rac Signaling |
| | macrophages STA | | | | |
| FEZ2 | CX3CR1+ lining | PLD3 | IL-8 Signaling | PIK3CB | Rac Signaling |
| | macrophages STA | | | | |
| FN1 | CX3CR1+ lining | PRKCA | IL-8 Signaling | PIK3CD | Rac Signaling |
| | macrophages STA | | | | |
| GAS6 | CX3CR1+ lining | PRKCB | IL-8 Signaling | PIK3CG | Rac Signaling |
| | macrophages STA | | | | |
| GNAS | CX3CR1+ lining | PRKCD | IL-8 Signaling | PIK3R1 | Rac Signaling |
| | macrophages STA | | | | |
| GNS | CX3CR1+ lining | PRKCI | IL-8 Signaling | PIK3R4 | Rac Signaling |
| | macrophages STA | | | | |
| GRN | CX3CR1+ lining | PRKD1 | IL-8 Signaling | PIP4K2A | Rac Signaling |
| | macrophages STA | | | | |
| HEXA | CX3CR1+ lining | PRKD3 | IL-8 Signaling | PRKCI | Rac Signaling |
| | macrophages STA | | | | |
| HLA-A | CX3CR1+ lining | PTK2 | IL-8 Signaling | PTK2B | Rac Signaling |
| | macrophages STA | | | | |
| HSBP1 | CX3CR1+ lining | PTK2B | IL-8 Signaling | RAC1 | Rac Signaling |
| | macrophages STA | | | | |
| IGF1 | CX3CR1+ lining | PTPN11 | IL-8 Signaling | RHOA | Rac Signaling |
| | macrophages STA | | | | |
| ITGAV | CX3CR1+ lining | RAC1 | IL-8 Signaling | RPS6KB1 | Rac Signaling |
| | macrophages STA | | | | |
| ITGB2 | CX3CR1+ lining | RAC2 | IL-8 Signaling | AHNAK | RELM-a interstitial |
| | macrophages STA | | | | macrophages STA |
| ITGB5 | CX3CR1+ lining | RACK1 | IL-8 Signaling | AHNAK2 | RELM-a interstitial |
| | macrophages STA | | | | macrophages STA |
| KLC1 | CX3CR1+ lining | RAF1 | IL-8 Signaling | ANP32A | RELM-a interstitial |
| | macrophages STA | | | | macrophages STA |
| LAIR1 | CX3CR1+ lining | RHOA | IL-8 Signaling | AQP1 | RELM-a interstitial |
| | macrophages STA | | | | macrophages STA |
| LPCAT2 | CX3CR1+ lining | RHOB | IL-8 Signaling | C16orf54 | RELM-a interstitial |
| | macrophages STA | | | | macrophages STA |
| LRP1 | CX3CR1+ lining | RHOH | IL-8 Signaling | C1QA | RELM-a interstitial |
| | macrophages STA | | | | macrophages STA |
| MAF | CX3CR1+ lining | RHOJ | IL-8 Signaling | C1QB | RELM-a interstitial |
| | macrophages STA | | | | macrophages STA |
| MAPK3 | CX3CR1+ lining | RHOQ | IL-8 Signaling | C1QC | RELM-a interstitial |
| | macrophages STA | | | | macrophages STA |
| MEF2C | CX3CR1+ lining | RHOT2 | IL-8 Signaling | CD37 | RELM-a interstitial |
| | macrophages STA | | | | macrophages STA |
| MS4A7 | CX3CR1+ lining | RHOU | IL-8 Signaling | CD48 | RELM-a interstitial |
| | macrophages STA | | | | macrophages STA |
| MTDH | CX3CR1+ lining | RND3 | IL-8 Signaling | CLN8 | RELM-a interstitial |
| | macrophages STA | | | | macrophages STA |
| MTUS1 | CX3CR1+ lining | ROCK1 | IL-8 Signaling | CLTC | RELM-a interstitial |
| | macrophages STA | | | | macrophages STA |
| NECAP2 | CX3CR1+ lining | ROCK2 | IL-8 Signaling | COMT | RELM-a interstitial |
| | macrophages STA | | | | macrophages STA |
| NFIA | CX3CR1+ lining | RPS6KB1 | IL-8 Signaling | CTSB | RELM-a interstitial |
| | macrophages STA | | | | macrophages STA |
| NPTN | CX3CR1+ lining | RRAS | IL-8 Signaling | CTSC | RELM-a interstitial |
| | macrophages STA | | | | macrophages STA |
| OLFML3 | CX3CR1+ lining | VASP | IL-8 Signaling | DOK2 | RELM-a interstitial |
| | macrophages STA | | | | macrophages STA |
| PDLIM7 | CX3CR1+ lining | VEGFB | IL-8 Signaling | DSTN | RELM-a interstitial |
| | macrophages STA | | | | macrophages STA |
| PMEPA1 | CX3CR1+ lining | CHUK | IL1 Pathway | FEZ2 | RELM-a interstitial |
| | macrophages STA | | | | macrophages STA |
| PON2 | CX3CR1+ lining | IKBKB | IL1 Pathway | FKBP1A | RELM-a interstitial |
| | macrophages STA | | | | macrophages STA |
| PRUNE2 | CX3CR1+ lining | NFKB1 | IL1 Pathway | FNTA | RELM-a interstitial |
| | macrophages STA | | | | macrophages STA |
| PTPRA | CX3CR1+ lining | MAP2K1 | IL1 Pathway | FOXN3 | RELM-a interstitial |
| | macrophages STA | | | | macrophages STA |
| QKI | CX3CR1+ lining | MAP2K4 | IL1 Pathway | GAS6 | RELM-a interstitial |
| | macrophages STA | | | | macrophages STA |
| QPCT | CX3CR1+ lining | IKBKG | IL1 Pathway | GAS7 | RELM-a interstitial |
| | macrophages STA | | | | macrophages STA |
| RAB11FIP5 | CX3CR1+ lining | IL1A | IL1 Pathway | H2AFV | RELM-a interstitial |
| | macrophages STA | | | | macrophages STA |
| RAB31 | CX3CR1+ lining | IL1R1 | IL1 Pathway | HCST | RELM-a interstitial |
| | macrophages STA | | | | macrophages STA |
| RENBP | CX3CR1+ lining | IRAK1 | IL1 Pathway | HEXA | RELM-a interstitial |
| | macrophages STA | | | | macrophages STA |
| RGS10 | CX3CR1+ lining | MAP3K3 | IL1 Pathway | HFE | RELM-a interstitial |
| | macrophages STA | | | | macrophages STA |
| RHOA | CX3CR1+ lining | MYD88 | IL1 Pathway | HP1BP3 | RELM-a interstitial |
| | macrophages STA | | | | macrophages STA |
| RHOB | CX3CR1+ lining | MAP2K6 | IL1 Pathway | IFI16 | RELM-a interstitial |
| | macrophages STA | | | | macrophages STA |
| RNASET2 | CX3CR1+ lining | MAP3K7 | IL1 Pathway | IGF1 | RELM-a interstitial |
| | macrophages STA | | | | macrophages STA |
| RNF13 | CX3CR1+ lining | TRAF6 | IL1 Pathway | IGFBP4 | RELM-a interstitial |
| | macrophages STA | | | | macrophages STA |
| SAMD9L | CX3CR1+ lining | TAB1 | IL1 Pathway | ITPRIPL2 | RELM-a interstitial |
| | macrophages STA | | | | macrophages STA |
| SASH1 | CX3CR1+ lining | TAB2 | IL1 Pathway | ITSN1 | RELM-a interstitial |
| | macrophages STA | | | | macrophages STA |
| SAT1 | CX3CR1+ lining | IRAK4 | IL1 Pathway | LEPROT | RELM-a interstitial |
| | macrophages STA | | | | macrophages STA |
| SELENBP1 | CX3CR1+ lining | TOLLIP | IL1 Pathway | LTC4S | RELM-a interstitial |
| | macrophages STA | | | | macrophages STA |
| SERINC3 | CX3CR1+ lining | MAP3K8 | IL1 Pathway | LY96 | RELM-a interstitial |
| | macrophages STA | | | | macrophages STA |
| SH3GLB1 | CX3CR1+ lining | IL1RAP | IL1 Pathway | MAF | RELM-a interstitial |
| | macrophages STA | | | | macrophages STA |
| SLCO2B1 | CX3CR1+ lining | IRAK2 | IL1 Pathway | MAFB | RELM-a interstitial |
| | macrophages STA | | | | macrophages STA |
| SMAGP | CX3CR1+ lining | MAPK8 | IL1 Pathway | MAN1A1 | RELM-a interstitial |
| | macrophages STA | | | | macrophages STA |
| SMIM14 | CX3CR1+ lining | RELA | IL1 Pathway | MBNL1 | RELM-a interstitial |
| | macrophages STA | | | | macrophages STA |
| SNX3 | CX3CR1+ lining | TAB3 | IL1 Pathway | MEF2C | RELM-a interstitial |
| | macrophages STA | | | | macrophages STA |
| SPARC | CX3CR1+ lining | IL1B | IL1 Pathway | MTSS1 | RELM-a interstitial |
| | macrophages STA | | | | macrophages STA |
| SRGAP2 | CX3CR1+ lining | UBE2N | IL1 Pathway | NCOA4 | RELM-a interstitial |
| | macrophages STA | | | | macrophages STA |
| STAB1 | CX3CR1+ lining | SQSTM1 | IL1 Pathway | NDFIP1 | RELM-a interstitial |
| | macrophages STA | | | | macrophages STA |
| STOM | CX3CR1+ lining | IRAK3 | IL1 Pathway | NENF | RELM-a interstitial |
| | macrophages STA | | | | macrophages STA |
| SULF2 | CX3CR1+ lining | IL6ST | IL6 Pathway | NISCH | RELM-a interstitial |
| | macrophages STA | | | | macrophages STA |
| TCN2 | CX3CR1+ lining | JAK1 | IL6 Pathway | PDLIM4 | RELM-a interstitial |
| | macrophages STA | | | | macrophages STA |
| TGFBR2 | CX3CR1+ lining | JAK2 | IL6 Pathway | PEA15 | RELM-a interstitial |
| | macrophages STA | | | | macrophages STA |
| TMEM50A | CX3CR1+ lining | TYK2 | IL6 Pathway | PINK1 | RELM-a interstitial |
| | macrophages STA | | | | macrophages STA |
| TMEM9B | CX3CR1+ lining | CNTF | IL6 Pathway | PON2 | RELM-a interstitial |
| | macrophages STA | | | | macrophages STA |
| TPP1 | CX3CR1+ lining | CNTFR | IL6 Pathway | PROS1 | RELM-a interstitial |
| | macrophages STA | | | | macrophages STA |
| TSPAN3 | CX3CR1+ lining | CTF1 | IL6 Pathway | PRUNE2 | RELM-a interstitial |
| | macrophages STA | | | | macrophages STA |
| ZFHX3 | CX3CR1+ lining | IL11 | IL6 Pathway | PTAFR | RELM-a interstitial |
| | macrophages STA | | | | macrophages STA |
| CD83 | CX3CR1+ lining | IL11RA | IL6 Pathway | PTS | RELM-a interstitial |
| | macrophages steady-state | | | | macrophages STA |
| FN1 | CX3CR1+ lining | IL12A | IL6 Pathway | QPCT | RELM-a interstitial |
| | macrophages steady-state | | | | macrophages STA |
| LTC4S | CX3CR1+ lining | IL12RB2 | IL6 Pathway | RAB11FIP5 | RELM-a interstitial |
| | macrophages steady-state | | | | macrophages STA |
| NFKBIA | CX3CR1+ lining | LIF | IL6 Pathway | RAB31 | RELM-a interstitial |
| | macrophages steady-state | | | | macrophages STA |
| SPARC | CX3CR1+ lining | LIFR | IL6 Pathway | RAPGEF6 | RELM-a interstitial |
| | macrophages steady-state | | | | macrophages STA |
| TIMP2 | CX3CR1+ lining | OSM | IL6 Pathway | RASSF4 | RELM-a interstitial |
| | macrophages steady-state | | | | macrophages STA |
| CD4 | CXCR4 Signaling | OSMR | IL6 Pathway | RFK | RELM-a interstitial |
| | | | | | macrophages STA |
| CXCR4 | CXCR4 Signaling | CRLF1 | IL6 Pathway | RGS10 | RELM-a interstitial |
| | | | | | macrophages STA |
| EGR1 | CXCR4 Signaling | IL27RA | IL6 Pathway | RNF13 | RELM-a interstitial |
| | | | | | macrophages STA |
| FGFR1 | CXCR4 Signaling | EBI3 | IL6 Pathway | RNF130 | RELM-a interstitial |
| | | | | | macrophages STA |
| FNBP1 | CXCR4 Signaling | CLCF1 | IL6 Pathway | SEC14L1 | RELM-a interstitial |
| | | | | | macrophages STA |
| FOS | CXCR4 Signaling | IL27 | IL6 Pathway | SEC62 | RELM-a interstitial |
| | | | | | macrophages STA |
| GNA13 | CXCR4 Signaling | CBL | IL6 Pathway | SELENBP1 | RELM-a interstitial |
| | | | | | macrophages STA |
| GNAI3 | CXCR4 Signaling | IL6 | IL6 Pathway | SERINC3 | RELM-a interstitial |
| | | | | | macrophages STA |
| GNAQ | CXCR4 Signaling | IL6R | IL6 Pathway | SERPINB1 | RELM-a interstitial |
| | | | | | macrophages STA |
| GNAS | CXCR4 Signaling | PTPN11 | IL6 Pathway | SH3BGRL | RELM-a interstitial |
| | | | | | macrophages STA |
| GNB1 | CXCR4 Signaling | STAT1 | IL6 Pathway | SLC48A1 | RELM-a interstitial |
| | | | | | macrophages STA |
| GNB4 | CXCR4 Signaling | STAT3 | IL6 Pathway | SLCO2B1 | RELM-a interstitial |
| | | | | | macrophages STA |
| GNG12 | CXCR4 Signaling | SOCS3 | IL6 Pathway | SMIM10L1 | RELM-a interstitial |
| | | | | | macrophages STA |
| GRB2 | CXCR4 Signaling | AIM2 | Inflammasome | SNX2 | RELM-a interstitial |
| | | | | | macrophages STA |
| ITPR1 | CXCR4 Signaling | CASP1 | Inflammasome | SNX3 | RELM-a interstitial |
| | | | | | macrophages STA |
| KRAS | CXCR4 Signaling | CASP5 | Inflammasome | SPRED1 | RELM-a interstitial |
| | | | | | macrophages STA |
| LYN | CXCR4 Signaling | CTSB | Inflammasome | STAB1 | RELM-a interstitial |
| | | | | | macrophages STA |
| MAPK1 | CXCR4 Signaling | NAIP | Inflammasome | SULF2 | RELM-a interstitial |
| | | | | | macrophages STA |
| MAPK3 | CXCR4 Signaling | NLRC4 | Inflammasome | TACC1 | RELM-a interstitial |
| | | | | | macrophages STA |
| PAK1 | CXCR4 Signaling | NLRP3 | Inflammasome | TCF4 | RELM-a interstitial |
| | | | | | macrophages STA |
| PAK2 | CXCR4 Signaling | NOD2 | Inflammasome | TCN2 | RELM-a interstitial |
| | | | | | macrophages STA |
| PIK3C2A | CXCR4 Signaling | PYCARD | Inflammasome | TGFBR2 | RELM-a interstitial |
| | | | | | macrophages STA |
| PIK3CB | CXCR4 Signaling | P2RX7 | Inflammasome | TIMP2 | RELM-a interstitial |
| | | | | | macrophages STA |
| PIK3CD | CXCR4 Signaling | NEK7 | Inflammasome | TMEM50A | RELM-a interstitial |
| | | | | | macrophages STA |
| PIK3CG | CXCR4 Signaling | NLRP1 | Inflammasome | TXNIP | RELM-a interstitial |
| | | | | | macrophages STA |
| PIK3R1 | CXCR4 Signaling | PANX1 | Inflammasome | UNC93B1 | RELM-a interstitial |
| | | | | | macrophages STA |
| PIK3R4 | CXCR4 Signaling | GSDMD | Inflammasome | VKORC1 | RELM-a interstitial |
| | | | | | macrophages STA |
| PLCB2 | CXCR4 Signaling | GSDMB | Inflammasome | WWP1 | RELM-a interstitial |
| | | | | | macrophages STA |
| PRKCB | CXCR4 Signaling | IL18 | Inflammasome | ZBTB20 | RELM-a interstitial |
| | | | | | macrophages STA |
| PRKCI | CXCR4 Signaling | IL1B | Inflammasome | CXCL13 | RELM-a interstitial |
| | | | | | macrophages steady- |
| | | | | | state |
| PRKD3 | CXCR4 Signaling | RIPK1 | Inflammasome | DUSP1 | RELM-a interstitial |
| | | | | | macrophages steady- |
| | | | | | state |
| RAC1 | CXCR4 Signaling | AIM2 | Inflammasome Pathway | FOS | RELM-a interstitial |
| | | | | | macrophages steady- |
| | | | | | state |
| RHOA | CXCR4 Signaling | CASP1 | Inflammasome Pathway | CXCL3 | RELM-a interstitial |
| | | | | | macrophages steady- |
| | | | | | state |
| RHOQ | CXCR4 Signaling | CASP8 | Inflammasome Pathway | JUN | RELM-a interstitial |
| | | | | | macrophages steady- |
| | | | | | state |
| ROCK1 | CXCR4 Signaling | CTSB | Inflammasome Pathway | MAF | RELM-a interstitial |
| | | | | | macrophages steady- |
| | | | | | state |
| CCL5 | Cytotoxic T Cells | MYD88 | Inflammasome Pathway | MCL1 | RELM-a interstitial |
| | | | | | macrophages steady- |
| | | | | | state |
| IL16 | Cytotoxic T Cells | NEK7 | Inflammasome Pathway | NFKBIA | RELM-a interstitial |
| | | | | | macrophages steady- |
| | | | | | state |
| GZMA | Cytotoxic T Cells | NFKB2 | Inflammasome Pathway | CCL4 | RELM-a interstitial |
| | | | | | macrophages steady- |
| | | | | | state |
| GZMH | Cytotoxic T Cells | NLRP1 | Inflammasome Pathway | CCL13 | RELM-a interstitial |
| | | | | | macrophages steady- |
| | | | | | state |
| PRF1 | Cytotoxic T Cells | NLRP3 | Inflammasome Pathway | UBC | RELM-a interstitial |
| | | | | | macrophages steady- |
| | | | | | state |
| CTSW | Cytotoxic T Cells | P2RX7 | Inflammasome Pathway | BTG2 | RELM-a interstitial |
| | | | | | macrophages steady- |
| | | | | | state |
| SRGN | Cytotoxic T Cells | PANX1 | Inflammasome Pathway | CD83 | RELM-a interstitial |
| | | | | | macrophages steady- |
| | | | | | state |
| STXBP2 | Cytotoxic T Cells | PYCARD | Inflammasome Pathway | KLF4 | RELM-a interstitial |
| | | | | | macrophages steady- |
| | | | | | state |
| IL12RB1 | Cytotoxic T Cells | TLR4 | Inflammasome Pathway | C3AR1 | Role of PRRs in |
| | | | | | Recognition of Bacteria |
| | | | | | and Viruses |
| FCER1G | Dendritic Cell Maturation | IL1A | Inflammatory Cytokines | CLEC7A | Role of PRRs in |
| | | | | | Recognition of Bacteria |
| | | | | | and Viruses |
| IRF8 | Dendritic Cell Maturation | CXCL10 | Inflammatory Cytokines | CXCL8 | Role of PRRs in |
| | | | | | Recognition of Bacteria |
| | | | | | and Viruses |
| LY75 | Dendritic Cell Maturation | CXCL11 | Inflammatory Cytokines | IL17D | Role of PRRs in |
| | | | | | Recognition of Bacteria |
| | | | | | and Viruses |
| IL10 | Dendritic Cell Maturation | CXCL9 | Inflammatory Cytokines | IL1A | Role of PRRs in |
| | | | | | Recognition of Bacteria |
| | | | | | and Viruses |
| AKT2 | Dendritic Cell Maturation | TNFSF13B | Inflammatory Cytokines | OAS1 | Role of PRRs in |
| | | | | | Recognition of Bacteria |
| | | | | | and Viruses |
| AKT3 | Dendritic Cell Maturation | CCL8 | Inflammatory Cytokines | OAS2 | Role of PRRs in |
| | | | | | Recognition of Bacteria |
| | | | | | and Viruses |
| ATF2 | Dendritic Cell Maturation | TNF | Inflammatory Cytokines | OAS3 | Role of PRRs in |
| | | | | | Recognition of Bacteria |
| | | | | | and Viruses |
| ATF4 | Dendritic Cell Maturation | IL1B | Inflammatory Cytokines | SYK | Role of PRRs in |
| | | | | | Recognition of Bacteria |
| | | | | | and Viruses |
| ATM | Dendritic Cell Maturation | IL18 | Inflammatory Cytokines | TGFB3 | Role of PRRs in |
| | | | | | Recognition of Bacteria |
| | | | | | and Viruses |
| B2M | Dendritic Cell Maturation | AOAH | Inhibitory Macs | TLR8 | Role of PRRs in |
| | | | | | Recognition of Bacteria |
| | | | | | and Viruses |
| CD40 | Dendritic Cell Maturation | BACH1 | Inhibitory Macs | EIF2S1 | Role of PRRs in |
| | | | | | Recognition of Bacteria |
| | | | | | and Viruses |
| CD58 | Dendritic Cell Maturation | CD200R1 | Inhibitory Macs | IL10 | Role of PRRs in |
| | | | | | Recognition of Bacteria |
| | | | | | and Viruses |
| CD80 | Dendritic Cell Maturation | CD300A | Inhibitory Macs | ATM | Role of PRRs in |
| | | | | | Recognition of Bacteria |
| | | | | | and Viruses |
| CD83 | Dendritic Cell Maturation | CD163 | Inhibitory Macs | C1QA | Role of PRRs in |
| | | | | | Recognition of Bacteria |
| | | | | | and Viruses |
| CD86 | Dendritic Cell Maturation | CLEC7A | Inhibitory Macs | C1QB | Role of PRRs in |
| | | | | | Recognition of Bacteria |
| | | | | | and Viruses |
| COL11A2 | Dendritic Cell Maturation | SCARB1 | Inhibitory Macs | C1QC | Role of PRRs in |
| | | | | | Recognition of Bacteria |
| | | | | | and Viruses |
| COL18A1 | Dendritic Cell Maturation | MS4A4A | Inhibitory Macs | CASP1 | Role of PRRs in |
| | | | | | Recognition of Bacteria |
| | | | | | and Viruses |
| COL1A1 | Dendritic Cell Maturation | IL10 | Inhibitory Macs | CCL5 | Role of PRRs in |
| | | | | | Recognition of Bacteria |
| | | | | | and Viruses |
| COL1A2 | Dendritic Cell Maturation | HAVCR2 | Inhibitory Receptors | CREB1 | Role of PRRs in |
| | | | | | Recognition of Bacteria |
| | | | | | and Viruses |
| COL3A1 | Dendritic Cell Maturation | MARCO | Inhibitory Receptors | DDX58 | Role of PRRs in |
| | | | | | Recognition of Bacteria |
| | | | | | and Viruses |
| COL5A3 | Dendritic Cell Maturation | LILRB1 | Inhibitory Receptors | EIF2AK2 | Role of PRRs in |
| | | | | | Recognition of Bacteria |
| | | | | | and Viruses |
| CREB1 | Dendritic Cell Maturation | LILRB2 | Inhibitory Receptors | FGFR1 | Role of PRRs in |
| | | | | | Recognition of Bacteria |
| | | | | | and Viruses |
| CREB5 | Dendritic Cell Maturation | LILRB3 | Inhibitory Receptors | FRS2 | Role of PRRs in |
| | | | | | Recognition of Bacteria |
| | | | | | and Viruses |
| CREBBP | Dendritic Cell Maturation | CD300A | Inhibitory Receptors | GAB1 | Role of PRRs in |
| | | | | | Recognition of Bacteria |
| | | | | | and Viruses |
| FCGR2A | Dendritic Cell Maturation | PILRA | Inhibitory Receptors | GRB2 | Role of PRRs in |
| | | | | | Recognition of Bacteria |
| | | | | | and Viruses |
| FCGR2C | Dendritic Cell Maturation | IRF9 | Interferon Signaling | IFIH1 | Role of PRRs in |
| | | | | | Recognition of Bacteria |
| | | | | | and Viruses |
| FCGR3A | Dendritic Cell Maturation | MED14 | Interferon Signaling | IL6 | Role of PRRs in |
| | | | | | Recognition of Bacteria |
| | | | | | and Viruses |
| FCGR3B | Dendritic Cell Maturation | PIAS1 | Interferon Signaling | IRF7 | Role of PRRs in |
| | | | | | Recognition of Bacteria |
| | | | | | and Viruses |
| FGFR1 | Dendritic Cell Maturation | BAX | Interferon Signaling | IRS1 | Role of PRRs in |
| | | | | | Recognition of Bacteria |
| | | | | | and Viruses |
| FRS2 | Dendritic Cell Maturation | IFI6 | Interferon Signaling | MAP2K4 | Role of PRRs in |
| | | | | | Recognition of Bacteria |
| | | | | | and Viruses |
| FSCN1 | Dendritic Cell Maturation | IFI35 | Interferon Signaling | MAPK1 | Role of PRRs in |
| | | | | | Recognition of Bacteria |
| | | | | | and Viruses |
| GAB1 | Dendritic Cell Maturation | IFIT1 | Interferon Signaling | MAPK3 | Role of PRRs in |
| | | | | | Recognition of Bacteria |
| | | | | | and Viruses |
| GRB2 | Dendritic Cell Maturation | IFIT3 | Interferon Signaling | MAPK8 | Role of PRRs in |
| | | | | | Recognition of Bacteria |
| | | | | | and Viruses |
| HLA-A | Dendritic Cell Maturation | IFITM1 | Interferon Signaling | MAPK9 | Role of PRRs in |
| | | | | | Recognition of Bacteria |
| | | | | | and Viruses |
| HLA-B | Dendritic Cell Maturation | IFNAR2 | Interferon Signaling | MAVS | Role of PRRs in |
| | | | | | Recognition of Bacteria |
| | | | | | and Viruses |
| HLA-C | Dendritic Cell Maturation | IFNGR1 | Interferon Signaling | MYD88 | Role of PRRs in |
| | | | | | Recognition of Bacteria |
| | | | | | and Viruses |
| HLA-DMA | Dendritic Cell Maturation | IRF1 | Interferon Signaling | NFKB2 | Role of PRRs in |
| | | | | | Recognition of Bacteria |
| | | | | | and Viruses |
| HLA-DMB | Dendritic Cell Maturation | ISG15 | Interferon Signaling | NLRP3 | Role of PRRs in |
| | | | | | Recognition of Bacteria |
| | | | | | and Viruses |
| HLA-DOA | Dendritic Cell Maturation | JAK1 | Interferon Signaling | NOD1 | Role of PRRs in |
| | | | | | Recognition of Bacteria |
| | | | | | and Viruses |
| HLA-DOB | Dendritic Cell Maturation | JAK2 | Interferon Signaling | PIK3C3 | Role of PRRs in |
| | | | | | Recognition of Bacteria |
| | | | | | and Viruses |
| HLA-DQB1 | Dendritic Cell Maturation | MX1 | Interferon Signaling | PIK3C2A | Role of PRRs in |
| | | | | | Recognition of Bacteria |
| | | | | | and Viruses |
| HLA-DRA | Dendritic Cell Maturation | OAS1 | Interferon Signaling | PIK3C2B | Role of PRRs in |
| | | | | | Recognition of Bacteria |
| | | | | | and Viruses |
| HLA-DRB1 | Dendritic Cell Maturation | PSMB8 | Interferon Signaling | PIK3CB | Role of PRRs in |
| | | | | | Recognition of Bacteria |
| | | | | | and Viruses |
| ICAM1 | Dendritic Cell Maturation | STAT1 | Interferon Signaling | PIK3CD | Role of PRRs in |
| | | | | | Recognition of Bacteria |
| | | | | | and Viruses |
| IGHG1 | Dendritic Cell Maturation | STAT2 | Interferon Signaling | PIK3CG | Role of PRRs in |
| | | | | | Recognition of Bacteria |
| | | | | | and Viruses |
| IKBKB | Dendritic Cell Maturation | TAP1 | Interferon Signaling | PIK3R1 | Role of PRRs in |
| | | | | | Recognition of Bacteria |
| | | | | | and Viruses |
| IKBKE | Dendritic Cell Maturation | BCL2L1 | Jak/Stat Signaling | PLCG2 | Role of PRRs in |
| | | | | | Recognition of Bacteria |
| | | | | | and Viruses |
| IL6 | Dendritic Cell Maturation | FGFR1 | Jak/Stat Signaling | PRKCA | Role of PRRs in |
| | | | | | Recognition of Bacteria |
| | | | | | and Viruses |
| IL15 | Dendritic Cell Maturation | GAB1 | Jak/Stat Signaling | PRKCB | Role of PRRs in |
| | | | | | Recognition of Bacteria |
| | | | | | and Viruses |
| IL32 | Dendritic Cell Maturation | JAK1 | Jak/Stat Signaling | PRKCD | Role of PRRs in |
| | | | | | Recognition of Bacteria |
| | | | | | and Viruses |
| IL1A | Dendritic Cell Maturation | JUN | Jak/Stat Signaling | PRKCI | Role of PRRs in |
| | | | | | Recognition of Bacteria |
| | | | | | and Viruses |
| IL1RN | Dendritic Cell Maturation | NFKB2 | Jak/Stat Signaling | PRKD1 | Role of PRRs in |
| | | | | | Recognition of Bacteria |
| | | | | | and Viruses |
| IRS1 | Dendritic Cell Maturation | NRAS | Jak/Stat Signaling | PRKD3 | Role of PRRs in |
| | | | | | Recognition of Bacteria |
| | | | | | and Viruses |
| JAK2 | Dendritic Cell Maturation | PIK3CG | Jak/Stat Signaling | PTPN11 | Role of PRRs in |
| | | | | | Recognition of Bacteria |
| | | | | | and Viruses |
| LTBR | Dendritic Cell Maturation | STAT1 | Jak/Stat Signaling | RIPK2 | Role of PRRs in |
| | | | | | Recognition of Bacteria |
| | | | | | and Viruses |
| MAP2K4 | Dendritic Cell Maturation | CEACAM4 | LDG | TLR1 | Role of PRRs in |
| | | | | | Recognition of Bacteria |
| | | | | | and Viruses |
| MAPK1 | Dendritic Cell Maturation | CXCL5 | LDG | TLR2 | Role of PRRs in |
| | | | | | Recognition of Bacteria |
| | | | | | and Viruses |
| MAPK3 | Dendritic Cell Maturation | PGLYRP1 | LDG | TLR3 | Role of PRRs in |
| | | | | | Recognition of Bacteria |
| | | | | | and Viruses |
| MAPK8 | Dendritic Cell Maturation | CHIT1 | LDG | TLR4 | Role of PRRs in |
| | | | | | Recognition of Bacteria |
| | | | | | and Viruses |
| MAPK9 | Dendritic Cell Maturation | CLEC5A | LDG | TLR6 | Role of PRRs in |
| | | | | | Recognition of Bacteria |
| | | | | | and Viruses |
| MAPK14 | Dendritic Cell Maturation | LTF | LDG | ACTR2 | Signaling by Rho |
| | | | | | Family GTPases |
| MYD88 | Dendritic Cell Maturation | OLR1 | LDG | ARHGEF7 | Signaling by Rho |
| | | | | | Family GTPases |
| NFKB2 | Dendritic Cell Maturation | OTOF | LDG | ARPC4 | Signaling by Rho |
| | | | | | Family GTPases |
| NFKBIA | Dendritic Cell Maturation | S100A8 | LDG | ARPC5 | Signaling by Rho |
| | | | | | Family GTPases |
| NFKBIE | Dendritic Cell Maturation | S100A9 | LDG | CDC42 | Signaling by Rho |
| | | | | | Family GTPases |
| NGFR | Dendritic Cell Maturation | ARG1 | LDG | CDC42EP3 | Signaling by Rho |
| | | | | | Family GTPases |
| PIK3C3 | Dendritic Cell Maturation | AZU1 | LDG | CFL1 | Signaling by Rho |
| | | | | | Family GTPases |
| PIK3C2A | Dendritic Cell Maturation | BPI | LDG | CLIP1 | Signaling by Rho |
| | | | | | Family GTPases |
| PIK3C2B | Dendritic Cell Maturation | CAMP | LDG | CYBB | Signaling by Rho |
| | | | | | Family GTPases |
| PIK3CB | Dendritic Cell Maturation | CEACAM5 | LDG | EZR | Signaling by Rho |
| | | | | | Family GTPases |
| PIK3CD | Dendritic Cell Maturation | CEACAM6 | LDG | FGFR1 | Signaling by Rho |
| | | | | | Family GTPases |
| PIK3CG | Dendritic Cell Maturation | CEACAM7 | LDG | FNBP1 | Signaling by Rho |
| | | | | | Family GTPases |
| PIK3R1 | Dendritic Cell Maturation | CEACAM8 | LDG | FOS | Signaling by Rho |
| | | | | | Family GTPases |
| PLCB1 | Dendritic Cell Maturation | CRISP3 | LDG | GNA13 | Signaling by Rho |
| | | | | | Family GTPases |
| PLCB4 | Dendritic Cell Maturation | CTSG | LDG | GNAI3 | Signaling by Rho |
| | | | | | Family GTPases |
| PLCD4 | Dendritic Cell Maturation | DEFA1 | LDG | GNAQ | Signaling by Rho |
| | | | | | Family GTPases |
| PLCE1 | Dendritic Cell Maturation | DEFA1B | LDG | GNAS | Signaling by Rho |
| | | | | | Family GTPases |
| PLCG1 | Dendritic Cell Maturation | DEFA3 | LDG | GNB1 | Signaling by Rho |
| | | | | | Family GTPases |
| PLCG2 | Dendritic Cell Maturation | DEFA4 | LDG | GNB4 | Signaling by Rho |
| | | | | | Family GTPases |
| PLCL2 | Dendritic Cell Maturation | DEFB103A | LDG | GNG12 | Signaling by Rho |
| | | | | | Family GTPases |
| PTPN11 | Dendritic Cell Maturation | DEFB103B | LDG | GRB2 | Signaling by Rho |
| | | | | | Family GTPases |
| STAT1 | Dendritic Cell Maturation | DEFB106B | LDG | IQGAP1 | Signaling by Rho |
| | | | | | Family GTPases |
| STAT2 | Dendritic Cell Maturation | DEFB136 | LDG | MAPK1 | Signaling by Rho |
| | | | | | Family GTPases |
| TLR2 | Dendritic Cell Maturation | DEFB4A | LDG | MAPK3 | Signaling by Rho |
| | | | | | Family GTPases |
| TLR3 | Dendritic Cell Maturation | ELANE | LDG | NCF2 | Signaling by Rho |
| | | | | | Family GTPases |
| TLR4 | Dendritic Cell Maturation | GRAP2 | LDG | NFKB2 | Signaling by Rho |
| | | | | | Family GTPases |
| TNFRSF11B | Dendritic Cell Maturation | LBP | LDG | PAK1 | Signaling by Rho |
| | | | | | Family GTPases |
| TNFRSF1B | Dendritic Cell Maturation | LCN2 | LDG | PAK2 | Signaling by Rho |
| | | | | | Family GTPases |
| ACTA2 | EIF2 Signaling | MMP8 | LDG | PIK3C2A | Signaling by Rho |
| | | | | | Family GTPases |
| ACTB | EIF2 Signaling | MPO | LDG | PIK3CB | Signaling by Rho |
| | | | | | Family GTPases |
| ACTG2 | EIF2 Signaling | MS4A3 | LDG | PIK3CD | Signaling by Rho |
| | | | | | Family GTPases |
| AGO1 | EIF2 Signaling | OLFM4 | LDG | PIK3CG | Signaling by Rho |
| | | | | | Family GTPases |
| AGO2 | EIF2 Signaling | OSM | LDG | PIK3R1 | Signaling by Rho |
| | | | | | Family GTPases |
| AGO3 | EIF2 Signaling | PRTN3 | LDG | PIK3R4 | Signaling by Rho |
| | | | | | Family GTPases |
| EIF1 | EIF2 Signaling | RETN | LDG | PIP4K2A | Signaling by Rho |
| | | | | | Family GTPases |
| EIF5 | EIF2 Signaling | RNASE3 | LDG | PRKCI | Signaling by Rho |
| | | | | | Family GTPases |
| EIF1AX | EIF2 Signaling | S100A12 | LDG | PTK2B | Signaling by Rho |
| | | | | | Family GTPases |
| EIF2S1 | EIF2 Signaling | CLDN12 | Leukocyte | RAC1 | Signaling by Rho |
| | | | Extravasation Signaling | | Family GTPases |
| EIF2S2 | EIF2 Signaling | CTTN | Leukocyte | RDX | Signaling by Rho |
| | | | Extravasation Signaling | | Family GTPases |
| EIF2S3 | EIF2 Signaling | CYBB | Leukocyte | RHOA | Signaling by Rho |
| | | | Extravasation Signaling | | Family GTPases |
| EIF3A | EIF2 Signaling | JAM2 | Leukocyte | RHOQ | Signaling by Rho |
| | | | Extravasation Signaling | | Family GTPases |
| EIF3B | EIF2 Signaling | JAM3 | Leukocyte | ROCK1 | Signaling by Rho |
| | | | Extravasation Signaling | | Family GTPases |
| EIF3D | EIF2 Signaling | RHOH | Leukocyte | SEPTIN2 | Signaling by Rho |
| | | | Extravasation Signaling | | Family GTPases |
| EIF3E | EIF2 Signaling | SELPLG | Leukocyte | SEPTIN7 | Signaling by Rho |
| | | | Extravasation Signaling | | Family GTPases |
| EIF3F | EIF2 Signaling | SPN | Leukocyte | SEPTIN11 | Signaling by Rho |
| | | | Extravasation Signaling | | Family GTPases |
| EIF3H | EIF2 Signaling | VCL | Leukocyte | WIPF1 | Signaling by Rho |
| | | | Extravasation Signaling | | Family GTPases |
| EIF3I | EIF2 Signaling | AFDN | Leukocyte | AGPAT5 | STMN1+ proliferating |
| | | | Extravasation Signaling | | cells STA |
| EIF3J | EIF2 Signaling | ARHGAP1 | Leukocyte | AIMP2 | STMN1+ proliferating |
| | | | Extravasation Signaling | | cells STA |
| EIF3K | EIF2 Signaling | ARHGAP4 | Leukocyte | AK6 | STMN1+ proliferating |
| | | | Extravasation Signaling | | cells STA |
| EIF3L | EIF2 Signaling | ARHGAP5 | Leukocyte | ANAPC5 | STMN1+ proliferating |
| | | | Extravasation Signaling | | cells STA |
| EIF3M | EIF2 Signaling | ARHGAP9 | Leukocyte | ANP32E | STMN1+ proliferating |
| | | | Extravasation Signaling | | cells STA |
| EIF4E | EIF2 Signaling | CDH5 | Leukocyte | AP1S1 | STMN1+ proliferating |
| | | | Extravasation Signaling | | cells STA |
| EIF4G1 | EIF2 Signaling | DLC1 | Leukocyte | ARL6IP1 | STMN1+ proliferating |
| | | | Extravasation Signaling | | cells STA |
| EIF4G2 | EIF2 Signaling | EDIL3 | Leukocyte | ARL8B | STMN1+ proliferating |
| | | | Extravasation Signaling | | cells STA |
| EIF4G3 | EIF2 Signaling | TIMP1 | Leukocyte | ARPP19 | STMN1+ proliferating |
| | | | Extravasation Signaling | | cells STA |
| HNRNPA1 | EIF2 Signaling | TIMP2 | Leukocyte | ATP5G1 | STMN1+ proliferating |
| | | | Extravasation Signaling | | cells STA |
| PAIP1 | EIF2 Signaling | TIMP3 | Leukocyte | BAG1 | STMN1+ proliferating |
| | | | Extravasation Signaling | | cells STA |
| RPS2 | EIF2 Signaling | VASP | Leukocyte | BAZIA | STMN1+ proliferating |
| | | | Extravasation Signaling | | cells STA |
| RPS3 | EIF2 Signaling | ACTA2 | Leukocyte | BICD2 | STMN1+ proliferating |
| | | | Extravasation Signaling | | cells STA |
| RPS9 | EIF2 Signaling | ACTB | Leukocyte | C1QBP | STMN1+ proliferating |
| | | | Extravasation Signaling | | cells STA |
| ATF4 | EIF2 Signaling | ACTG2 | Leukocyte | CBFB | STMN1+ proliferating |
| | | | Extravasation Signaling | | cells STA |
| ATF5 | EIF2 Signaling | ATM | Leukocyte | CBX1 | STMN1+ proliferating |
| | | | Extravasation Signaling | | cells STA |
| DDIT3 | EIF2 Signaling | BCAR1 | Leukocyte | CBX5 | STMN1+ proliferating |
| | | | Extravasation Signaling | | cells STA |
| GSK3B | EIF2 Signaling | BTK | Leukocyte | CCDC25 | STMN1+ proliferating |
| | | | Extravasation Signaling | | cells STA |
| HSPA5 | EIF2 Signaling | CD44 | Leukocyte | CCDC88A | STMN1+ proliferating |
| | | | Extravasation Signaling | | cells STA |
| AKT2 | EIF2 Signaling | CDC42 | Leukocyte | CCND1 | STMN1+ proliferating |
| | | | Extravasation Signaling | | cells STA |
| AKT3 | EIF2 Signaling | CRK | Leukocyte | CCT2 | STMN1+ proliferating |
| | | | Extravasation Signaling | | cells STA |
| ATM | EIF2 Signaling | CTNNA1 | Leukocyte | CCT3 | STMN1+ proliferating |
| | | | Extravasation Signaling | | cells STA |
| CCND1 | EIF2 Signaling | CXCR4 | Leukocyte | CCT7 | STMN1+ proliferating |
| | | | Extravasation Signaling | | cells STA |
| EIF2AK2 | EIF2 Signaling | CYBA | Leukocyte | CCT8 | STMN1+ proliferating |
| | | | Extravasation Signaling | | cells STA |
| EIF2AK4 | EIF2 Signaling | EZR | Leukocyte | CD44 | STMN1+ proliferating |
| | | | Extravasation Signaling | | cells STA |
| EIF2B1 | EIF2 Signaling | F11R | Leukocyte | CISD1 | STMN1+ proliferating |
| | | | Extravasation Signaling | | cells STA |
| EIF2B3 | EIF2 Signaling | FER | Leukocyte | CITED2 | STMN1+ proliferating |
| | | | Extravasation Signaling | | cells STA |
| EIF2B4 | EIF2 Signaling | FGFR1 | Leukocyte | CLIC4 | STMN1+ proliferating |
| | | | Extravasation Signaling | | cells STA |
| EIF2B5 | EIF2 Signaling | FRS2 | Leukocyte | COQ7 | STMN1+ proliferating |
| | | | Extravasation Signaling | | cells STA |
| EIF5B | EIF2 Signaling | GAB1 | Leukocyte | DAZAP1 | STMN1+ proliferating |
| | | | Extravasation Signaling | | cells STA |
| FGFR1 | EIF2 Signaling | GRB2 | Leukocyte | DDX21 | STMN1+ proliferating |
| | | | Extravasation Signaling | | cells STA |
| FRS2 | EIF2 Signaling | ICAM1 | Leukocyte | DEK | STMN1+ proliferating |
| | | | Extravasation Signaling | | cells STA |
| GAB1 | EIF2 Signaling | IRS1 | Leukocyte | DENR | STMN1+ proliferating |
| | | | Extravasation Signaling | | cells STA |
| GRB2 | EIF2 Signaling | ITGA1 | Leukocyte | DHX9 | STMN1+ proliferating |
| | | | Extravasation Signaling | | cells STA |
| HRAS | EIF2 Signaling | ITGA3 | Leukocyte | DNAJC2 | STMN1+ proliferating |
| | | | Extravasation Signaling | | cells STA |
| IGF1R | EIF2 Signaling | ITGA4 | Leukocyte | DPY30 | STMN1+ proliferating |
| | | | Extravasation Signaling | | cells STA |
| IRS1 | EIF2 Signaling | ITGA6 | Leukocyte | DYNLL2 | STMN1+ proliferating |
| | | | Extravasation Signaling | | cells STA |
| KRAS | EIF2 Signaling | ITGAL | Leukocyte | DYNLT1 | STMN1+ proliferating |
| | | | Extravasation Signaling | | cells STA |
| MAP2K1 | EIF2 Signaling | ITGB1 | Leukocyte | EIF2S2 | STMN1+ proliferating |
| | | | Extravasation Signaling | | cells STA |
| MAP2K2 | EIF2 Signaling | ITGB2 | Leukocyte | EIF4G2 | STMN1+ proliferating |
| | | | Extravasation Signaling | | cells STA |
| MAPK1 | EIF2 Signaling | MAP2K2 | Leukocyte | EIF6 | STMN1+ proliferating |
| | | | Extravasation Signaling | | cells STA |
| MAPK3 | EIF2 Signaling | MAP2K4 | Leukocyte | FBL | STMN1+ proliferating |
| | | | Extravasation Signaling | | cells STA |
| NRAS | EIF2 Signaling | MAP3K4 | Leukocyte | FKBP4 | STMN1+ proliferating |
| | | | Extravasation Signaling | | cells STA |
| PDPK1 | EIF2 Signaling | MAPK1 | Leukocyte | FXN | STMN1+ proliferating |
| | | | Extravasation Signaling | | cells STA |
| PIK3C3 | EIF2 Signaling | MAPK8 | Leukocyte | G3BP1 | STMN1+ proliferating |
| | | | Extravasation Signaling | | cells STA |
| PIK3C2A | EIF2 Signaling | MAPK9 | Leukocyte | GADD45GIP1 | STMN1+ proliferating |
| | | | Extravasation Signaling | | cells STA |
| PIK3C2B | EIF2 Signaling | MAPK14 | Leukocyte | GAS2L3 | STMN1+ proliferating |
| | | | Extravasation Signaling | | cells STA |
| PIK3CB | EIF2 Signaling | MMP1 | Leukocyte | GJA1 | STMN1+ proliferating |
| | | | Extravasation Signaling | | cells STA |
| PIK3CD | EIF2 Signaling | MYL6 | Leukocyte | GSPT1 | STMN1+ proliferating |
| | | | Extravasation Signaling | | cells STA |
| PIK3CG | EIF2 Signaling | NCF2 | Leukocyte | H2AFV | STMN1+ proliferating |
| | | | Extravasation Signaling | | cells STA |
| PIK3R1 | EIF2 Signaling | NCF4 | Leukocyte | H2AFY | STMN1+ proliferating |
| | | | Extravasation Signaling | | cells STA |
| PPP1CB | EIF2 Signaling | PECAM1 | Leukocyte | HDAC2 | STMN1+ proliferating |
| | | | Extravasation Signaling | | cells STA |
| PPP1R15A | EIF2 Signaling | PIK3C3 | Leukocyte | HMGB1 | STMN1+ proliferating |
| | | | Extravasation Signaling | | cells STA |
| PTPN11 | EIF2 Signaling | PIK3C2A | Leukocyte | HMGB2 | STMN1+ proliferating |
| | | | Extravasation Signaling | | cells STA |
| RAF1 | EIF2 Signaling | PIK3C2B | Leukocyte | HNRNPA0 | STMN1+ proliferating |
| | | | Extravasation Signaling | | cells STA |
| RPL3 | EIF2 Signaling | PIK3CB | Leukocyte | HNRNPAB | STMN1+ proliferating |
| | | | Extravasation Signaling | | cells STA |
| RPL4 | EIF2 Signaling | PIK3CD | Leukocyte | HNRNPM | STMN1+ proliferating |
| | | | Extravasation Signaling | | cells STA |
| RPL5 | EIF2 Signaling | PIK3CG | Leukocyte | HNRNPU | STMN1+ proliferating |
| | | | Extravasation Signaling | | cells STA |
| RPL6 | EIF2 Signaling | PIK3R1 | Leukocyte | HP1BP3 | STMN1+ proliferating |
| | | | Extravasation Signaling | | cells STA |
| RPL7 | EIF2 Signaling | PLCG1 | Leukocyte | HSP90AA1 | STMN1+ proliferating |
| | | | Extravasation Signaling | | cells STA |
| RPL8 | EIF2 Signaling | PLCG2 | Leukocyte | HSPA14 | STMN1+ proliferating |
| | | | Extravasation Signaling | | cells STA |
| RPL11 | EIF2 Signaling | PRKCA | Leukocyte | HUWE1 | STMN1+ proliferating |
| | | | Extravasation Signaling | | cells STA |
| RPL12 | EIF2 Signaling | PRKCB | Leukocyte | ILF3 | STMN1+ proliferating |
| | | | Extravasation Signaling | | cells STA |
| RPL14 | EIF2 Signaling | PRKCD | Leukocyte | IMMT | STMN1+ proliferating |
| | | | Extravasation Signaling | | cells STA |
| RPL15 | EIF2 Signaling | PRKCI | Leukocyte | IMPDH2 | STMN1+ proliferating |
| | | | Extravasation Signaling | | cells STA |
| RPL18 | EIF2 Signaling | PRKD1 | Leukocyte | IPO5 | STMN1+ proliferating |
| | | | Extravasation Signaling | | cells STA |
| RPL19 | EIF2 Signaling | PRKD3 | Leukocyte | ITGAV | STMN1+ proliferating |
| | | | Extravasation Signaling | | cells STA |
| RPL22 | EIF2 Signaling | PTK2 | Leukocyte | ITPA | STMN1+ proliferating |
| | | | Extravasation Signaling | | cells STA |
| RPL24 | EIF2 Signaling | PTK2B | Leukocyte | KDELR2 | STMN1+ proliferating |
| | | | Extravasation Signaling | | cells STA |
| RPL27 | EIF2 Signaling | PTPN11 | Leukocyte | KHSRP | STMN1+ proliferating |
| | | | Extravasation Signaling | | cells STA |
| RPL29 | EIF2 Signaling | RAC1 | Leukocyte | KPNA3 | STMN1+ proliferating |
| | | | Extravasation Signaling | | cells STA |
| RPL31 | EIF2 Signaling | RAC2 | Leukocyte | KPNB1 | STMN1+ proliferating |
| | | | Extravasation Signaling | | cells STA |
| RPL34 | EIF2 Signaling | RAP1A | Leukocyte | KTN1 | STMN1+ proliferating |
| | | | Extravasation Signaling | | cells STA |
| RPL37 | EIF2 Signaling | RAP1B | Leukocyte | LUC7L3 | STMN1+ proliferating |
| | | | Extravasation Signaling | | cells STA |
| RPL10A | EIF2 Signaling | RASGRP | Leukocyte | M6PR | STMN1+ proliferating |
| | | | Extravasation Signaling | | cells STA |
| RPL35A | EIF2 Signaling | RASSF5 | Leukocyte | MAP4K4 | STMN1+ proliferating |
| | | | Extravasation Signaling | | cells STA |
| RPL36A | EIF2 Signaling | RDX | Leukocyte | MAPK1 | STMN1+ proliferating |
| | | | Extravasation Signaling | | cells STA |
| RPL37A | EIF2 Signaling | RHOA | Leukocyte | MATR3 | STMN1+ proliferating |
| | | | Extravasation Signaling | | cells STA |
| RPLP0 | EIF2 Signaling | ROCK1 | Leukocyte | MDH2 | STMN1+ proliferating |
| | | | Extravasation Signaling | | cells STA |
| RPLP1 | EIF2 Signaling | ROCK2 | Leukocyte | MINOS1 | STMN1+ proliferating |
| | | | Extravasation Signaling | | cells STA |
| RPLP2 | EIF2 Signaling | THY1 | Leukocyte | MIS12 | STMN1+ proliferating |
| | | | Extravasation Signaling | | cells STA |
| RPS6 | EIF2 Signaling | VAV2 | Leukocyte | MIS18A | STMN1+ proliferating |
| | | | Extravasation Signaling | | cells STA |
| CBL | Fc.gamma.R-mediated | WAS | Leukocyte | MIS18BP1 | STMN1+ proliferating |
| | Phagocytosis in | | Extravasation Signaling | | cells STA |
| | Monos/Macs | | | | |
| ACTA2 | Fc.gamma.R-mediated | WASL | Leukocyte | MRPL49 | STMN1+ proliferating |
| | Phagocytosis in | | Extravasation Signaling | | cells STA |
| | Monos/Macs | | | | |
| ACTB | Fc.gamma.R-mediated | WIPF1 | Leukocyte | MRPL51 | STMN1+ proliferating |
| | Phagocytosis in | | Extravasation Signaling | | cells STA |
| | Monos/Macs | | | | |
| ACTG2 | Fc.gamma.R-mediated | AIM2 | M1 | MRPS17 | STMN1+ proliferating |
| | Phagocytosis in | | | | cells STA |
| | Monos/Macs | | | | |
| ACTR2 | Fc.gamma.R-mediated | ATF1 | M1 | MRPS25 | STMN1+ proliferating |
| | Phagocytosis in | | | | cells STA |
| | Monos/Macs | | | | |
| ACTR3 | Fc.gamma.R-mediated | BIRC3 | M1 | NAA50 | STMN1+ proliferating |
| | Phagocytosis in | | | | cells STA |
| | Monos/Macs | | | | |
| AKT2 | Fc.gamma.R-mediated | CCR1 | M1 | NAP1L1 | STMN1+ proliferating |
| | Phagocytosis in | | | | cells STA |
| | Monos/Macs | | | | |
| AKT3 | Fc.gamma.R-mediated | CXCL10 | M1 | NASP | STMN1+ proliferating |
| | Phagocytosis in | | | | cells STA |
| | Monos/Macs | | | | |
| ARF6 | Fc.gamma.R-mediated | FAS | M1 | NDUFA12 | STMN1+ proliferating |
| | Phagocytosis in | | | | cells STA |
| | Monos/Macs | | | | |
| ARPC2 | Fc.gamma.R-mediated | HLA-A | M1 | NFATC1 | STMN1+ proliferating |
| | Phagocytosis in | | | | cells STA |
| | Monos/Macs | | | | |
| ARPC4 | Fc.gamma.R-mediated | HLA-E | M1 | NHP2 | STMN1+ proliferating |
| | Phagocytosis in | | | | cells STA |
| | Monos/Macs | | | | |
| ARPC5 | Fc.gamma.R-mediated | IFI16 | M1 | NOL7 | STMN1+ proliferating |
| | Phagocytosis in | | | | cells STA |
| | Monos/Macs | | | | |
| ARPC1A | Fc.gamma.R-mediated | IFIH1 | M1 | NONO | STMN1+ proliferating |
| | Phagocytosis in | | | | cells STA |
| | Monos/Macs | | | | |
| ARPC5L | Fc.gamma.R-mediated | IFNGR2 | M1 | NOP10 | STMN1+ proliferating |
| | Phagocytosis in | | | | cells STA |
| | Monos/Macs | | | | |
| CDC42 | Fc.gamma.R-mediated | IL15 | M1 | NOP58 | STMN1+ proliferating |
| | Phagocytosis in | | | | cells STA |
| | Monos/Macs | | | | |
| CRK | Fc.gamma.R-mediated | IL15RA | M1 | NPM3 | STMN1+ proliferating |
| | Phagocytosis in | | | | cells STA |
| | Monos/Macs | | | | |
| EZR | Fc.gamma.R-mediated | IRF7 | M1 | NSMCE1 | STMN1+ proliferating |
| | Phagocytosis in | | | | cells STA |
| | Monos/Macs | | | | |
| FCGR2A | Fc.gamma.R-mediated | PSMB9 | M1 | NUCKS1 | STMN1+ proliferating |
| | Phagocytosis in | | | | cells STA |
| | Monos/Macs | | | | |
| FCGR3A | Fc.gamma.R-mediated | SLC31A2 | M1 | NUDC | STMN1+ proliferating |
| | Phagocytosis in | | | | cells STA |
| | Monos/Macs | | | | |
| FCGR3B | Fc.gamma.R-mediated | AK3 | M1 | NUDT5 | STMN1+ proliferating |
| | Phagocytosis in | | | | cells STA |
| | Monos/Macs | | | | |
| FGR | Fc.gamma.R-mediated | APOL1 | M1 | NUMA1 | STMN1+ proliferating |
| | Phagocytosis in | | | | cells STA |
| | Monos/Macs | | | | |
| FYB1 | Fc.gamma.R-mediated | APOL2 | M1 | NUP85 | STMN1+ proliferating |
| | Phagocytosis in | | | | cells STA |
| | Monos/Macs | | | | |
| INPP5D | Fc.gamma.R-mediated | APOL3 | M1 | ODC1 | STMN1+ proliferating |
| | Phagocytosis in | | | | cells STA |
| | Monos/Macs | | | | |
| LCP2 | Fc.gamma.R-mediated | APOL6 | M1 | ODF2 | STMN1+ proliferating |
| | Phagocytosis in | | | | cells STA |
| | Monos/Macs | | | | |
| LYN | Fc.gamma.R-mediated | ATF3 | M1 | OXCT1 | STMN1+ proliferating |
| | Phagocytosis in | | | | cells STA |
| | Monos/Macs | | | | |
| MAPK1 | Fc.gamma.R-mediated | BCL2A1 | M1 | PAICS | STMN1+ proliferating |
| | Phagocytosis in | | | | cells STA |
| | Monos/Macs | | | | |
| MAPK3 | Fc.gamma.R-mediated | CCL19 | M1 | PARK7 | STMN1+ proliferating |
| | Phagocytosis in | | | | cells STA |
| | Monos/Macs | | | | |
| MYO5A | Fc.gamma.R-mediated | CCL20 | M1 | PBRM1 | STMN1+ proliferating |
| | Phagocytosis in | | | | cells STA |
| | Monos/Macs | | | | |
| NAPEPLD | Fc.gamma.R-mediated | CCL5 | M1 | PCBD2 | STMN1+ proliferating |
| | Phagocytosis in | | | | cells STA |
| | Monos/Macs | | | | |
| NCK1 | Fc.gamma.R-mediated | CCR7 | M1 | PDPK1 | STMN1+ proliferating |
| | Phagocytosis in | | | | cells STA |
| | Monos/Macs | | | | |
| PAK1 | Fc.gamma.R-mediated | CHI3L2 | M1 | PDS5A | STMN1+ proliferating |
| | Phagocytosis in | | | | cells STA |
| | Monos/Macs | | | | |
| PIK3CG | Fc.gamma.R-mediated | VCAN | M1 | PNN | STMN1+ proliferating |
| | Phagocytosis in | | | | cells STA |
| | Monos/Macs | | | | |
| PIK3R1 | Fc.gamma.R-mediated | CXCL11 | M1 | POLE4 | STMN1+ proliferating |
| | Phagocytosis in | | | | cells STA |
| | Monos/Macs | | | | |
| PIP5K1A | Fc.gamma.R-mediated | CXCL9 | M1 | POLR2F | STMN1+ proliferating |
| | Phagocytosis in | | | | cells STA |
| | Monos/Macs | | | | |
| PLCG1 | Fc.gamma.R-mediated | TYMP | M1 | PPA1 | STMN1+ proliferating |
| | Phagocytosis in | | | | cells STA |
| | Monos/Macs | | | | |
| PLD3 | Fc.gamma.R-mediated | EDN1 | M1 | PPID | STMN1+ proliferating |
| | Phagocytosis in | | | | cells STA |
| | Monos/Macs | | | | |
| PRKCA | Fc.gamma.R-mediated | GADD45G | M1 | PPP2R5C | STMN1+ proliferating |
| | Phagocytosis in | | | | cells STA |
| | Monos/Macs | | | | |
| PRKCB | Fc.gamma.R-mediated | HESX1 | M1 | PRIM1 | STMN1+ proliferating |
| | Phagocytosis in | | | | cells STA |
| | Monos/Macs | | | | |
| PRKCD | Fc.gamma.R-mediated | HSD11B1 | M1 | PRKAR2A | STMN1+ proliferating |
| | Phagocytosis in | | | | cells STA |
| | Monos/Macs | | | | |
| PRKCI | Fc.gamma.R-mediated | XAF1 | M1 | PSMD1 | STMN1+ proliferating |
| | Phagocytosis in | | | | cells STA |
| | Monos/Macs | | | | |
| PRKD1 | Fc.gamma.R-mediated | IGFBP4 | M1 | PTMA | STMN1+ proliferating |
| | Phagocytosis in | | | | cells STA |
| | Monos/Macs | | | | |
| PRKD3 | Fc.gamma.R-mediated | IL1A | M1 | RAD21 | STMN1+ proliferating |
| | Phagocytosis in | | | | cells STA |
| | Monos/Macs | | | | |
| PTEN | Fc.gamma.R-mediated | IL12B | M1 | RAN | STMN1+ proliferating |
| | Phagocytosis in | | | | cells STA |
| | Monos/Macs | | | | |
| PTK2B | Fc.gamma.R-mediated | IL2 | M1 | RBBP4 | STMN1+ proliferating |
| | Phagocytosis in | | | | cells STA |
| | Monos/Macs | | | | |
| RAC1 | Fc.gamma.R-mediated | IL2RA | M1 | RDX | STMN1+ proliferating |
| | Phagocytosis in | | | | cells STA |
| | Monos/Macs | | | | |
| RAC2 | Fc.gamma.R-mediated | IL6 | M1 | RFC5 | STMN1+ proliferating |
| | Phagocytosis in | | | | cells STA |
| | Monos/Macs | | | | |
| RPS6KB1 | Fc.gamma.R-mediated | IL7R | M1 | RNASEH2C | STMN1+ proliferating |
| | Phagocytosis in | | | | cells STA |
| | Monos/Macs | | | | |
| SYK | Fc.gamma.R-mediated | IDO1 | M1 | RNF19B | STMN1+ proliferating |
| | Phagocytosis in | | | | cells STA |
| | Monos/Macs | | | | |
| TLN2 | Fc.gamma.R-mediated | INHBA | M1 | RNF7 | STMN1+ proliferating |
| | Phagocytosis in | | | | cells STA |
| | Monos/Macs | | | | |
| VAMP3 | Fc.gamma.R-mediated | IRF1 | M1 | RNPS1 | STMN1+ proliferating |
| | Phagocytosis in | | | | cells STA |
| | Monos/Macs | | | | |
| VASP | Fc.gamma.R-mediated | NAMPT | M1 | ROCK2 | STMN1+ proliferating |
| | Phagocytosis in | | | | cells STA |
| | Monos/Macs | | | | |
| VAV2 | Fc.gamma.R-mediated | PDGFA | M1 | RPA1 | STMN1+ proliferating |
| | Phagocytosis in | | | | cells STA |
| | Monos/Macs | | | | |
| WAS | Fc.gamma.R-mediated | PFKFB3 | M1 | RPL10A | STMN1+ proliferating |
| | Phagocytosis in | | | | cells STA |
| | Monos/Macs | | | | |
| YES1 | Fc.gamma.R-mediated | PFKP | M1 | RPL12 | STMN1+ proliferating |
| | Phagocytosis in | | | | cells STA |
| | Monos/Macs | | | | |
| CXCL13 | FDC | PLA1A | M1 | RPL14 | STMN1+ proliferating |
| | | | | | cells STA |
| CD72 | FDC | PSMA2 | M1 | RPL18 | STMN1+ proliferating |
| | | | | | cells STA |
| VCAM1 | FDC | PTX3 | M1 | RPL35A | STMN1+ proliferating |
| | | | | | cells STA |
| FDCSP | FDC | SLCO5A1 | M1 | RPLP1 | STMN1+ proliferating |
| | | | | | cells STA |
| FCER2 | FDC | SLC2A6 | M1 | RPS15 | STMN1+ proliferating |
| | | | | | cells STA |
| ADAM33 | Fibroblasts | SLC7A5 | M1 | RPS15A | STMN1+ proliferating |
| | | | | | cells STA |
| ADAMTS1 | Fibroblasts | SPHK1 | M1 | RPS2 | STMN1+ proliferating |
| | | | | | cells STA |
| ADAMTS6 | Fibroblasts | TNF | M1 | RPS24 | STMN1+ proliferating |
| | | | | | cells STA |
| ADGRA2 | Fibroblasts | TNFSF10 | M1 | SAE1 | STMN1+ proliferating |
| | | | | | cells STA |
| ADRAID | Fibroblasts | CTSC | M2 | SDHD | STMN1+ proliferating |
| | | | | | cells STA |
| AGTR1 | Fibroblasts | DAB2 | M2 | SEC11C | STMN1+ proliferating |
| | | | | | cells STA |
| ALPK2 | Fibroblasts | IL1RN | M2 | SET | STMN1+ proliferating |
| | | | | | cells STA |
| AMPH | Fibroblasts | MS4A4A | M2 | SF3B5 | STMN1+ proliferating |
| | | | | | cells STA |
| ANGPTL2 | Fibroblasts | ADK | M2 | SFXN1 | STMN1+ proliferating |
| | | | | | cells STA |
| ANKRD45 | Fibroblasts | ALOX15 | M2 | SHMT2 | STMN1+ proliferating |
| | | | | | cells STA |
| ANO2 | Fibroblasts | CA2 | M2 | SMC3 | STMN1+ proliferating |
| | | | | | cells STA |
| ANPEP | Fibroblasts | CCL13 | M2 | SNRNP27 | STMN1+ proliferating |
| | | | | | cells STA |
| APCDD1 | Fibroblasts | CCL18 | M2 | SNRNP70 | STMN1+ proliferating |
| | | | | | cells STA |
| ARID5B | Fibroblasts | CCL23 | M2 | SNRPD1 | STMN1+ proliferating |
| | | | | | cells STA |
| ARMC9 | Fibroblasts | CD36 | M2 | SPCS3 | STMN1+ proliferating |
| | | | | | cells STA |
| ARMCX1 | Fibroblasts | CERK | M2 | SRRM1 | STMN1+ proliferating |
| | | | | | cells STA |
| ARRDC4 | Fibroblasts | CHN2 | M2 | SRSF10 | STMN1+ proliferating |
| | | | | | cells STA |
| ASPN | Fibroblasts | CLEC10A | M2 | SRSF3 | STMN1+ proliferating |
| | | | | | cells STA |
| BDKRB2 | Fibroblasts | CXCR4 | M2 | SRSF7 | STMN1+ proliferating |
| | | | | | cells STA |
| BDNF | Fibroblasts | CD209 | M2 | SSRP1 | STMN1+ proliferating |
| | | | | | cells STA |
| BMPER | Fibroblasts | CLEC7A | M2 | STUB1 | STMN1+ proliferating |
| | | | | | cells STA |
| BOC | Fibroblasts | EGR2 | M2 | SUPT16H | STMN1+ proliferating |
| | | | | | cells STA |
| BTN3A3 | Fibroblasts | FGL2 | M2 | SYNCRIP | STMN1+ proliferating |
| | | | | | cells STA |
| C11orf87 | Fibroblasts | FN1 | M2 | TCF3 | STMN1+ proliferating |
| | | | | | cells STA |
| C19orf66 | Fibroblasts | GAS7 | M2 | TIMM13 | STMN1+ proliferating |
| | | | | | cells STA |
| C1QTNF1 | Fibroblasts | P2RY13 | M2 | TIMM50 | STMN1+ proliferating |
| | | | | | cells STA |
| C1S | Fibroblasts | HEXB | M2 | TMEM97 | STMN1+ proliferating |
| | | | | | cells STA |
| C3 | Fibroblasts | HNMT | M2 | TMPO | STMN1+ proliferating |
| | | | | | cells STA |
| C4orf22 | Fibroblasts | HRH1 | M2 | TUBB2A | STMN1+ proliferating |
| | | | | | cells STA |
| C5orf30 | Fibroblasts | HS3ST1 | M2 | UBALD2 | STMN1+ proliferating |
| | | | | | cells STA |
| CATSPER3 | Fibroblasts | HS3ST2 | M2 | UCHL5 | STMN1+ proliferating |
| | | | | | cells STA |
| CCDC102B | Fibroblasts | IGF1 | M2 | UQCRFS1 | STMN1+ proliferating |
| | | | | | cells STA |
| CCDC148 | Fibroblasts | LIPA | M2 | UQCRQ | STMN1+ proliferating |
| | | | | | cells STA |
| CCDC81 | Fibroblasts | LTA4H | M2 | USP1 | STMN1+ proliferating |
| | | | | | cells STA |
| CD248 | Fibroblasts | MAF | M2 | VARS | STMN1+ proliferating |
| | | | | | cells STA |
| CDR1 | Fibroblasts | MRC1 | M2 | VMA21 | STMN1+ proliferating |
| | | | | | cells STA |
| CEMIP | Fibroblasts | MS4A6A | M2 | VRK1 | STMN1+ proliferating |
| | | | | | cells STA |
| CHAC1 | Fibroblasts | MSR1 | M2 | YWHAH | STMN1+ proliferating |
| | | | | | cells STA |
| CHRM2 | Fibroblasts | P2RY14 | M2 | GAPDH | STMN1+ proliferating |
| | | | | | cells steady-state |
| CKAP4 | Fibroblasts | LPAR6 | M2 | LDHA | STMN1+ proliferating |
| | | | | | cells steady-state |
| CLMP | Fibroblasts | SELENOP | M2 | FBLN1 | Synovial CD34+ |
| | | | | | sublining fibroblasts |
| CNN1 | Fibroblasts | SLCO2B1 | M2 | GAS6 | Synovial CD34+ |
| | | | | | sublining fibroblasts |
| CNTN3 | Fibroblasts | SLC38A6 | M2 | GSN | Synovial CD34+ |
| | | | | | sublining fibroblasts |
| COL1A2 | Fibroblasts | SLC4A7 | M2 | IGF1 | Synovial CD34+ |
| | | | | | sublining fibroblasts |
| COL5A1 | Fibroblasts | TGFBI | M2 | PDGFRL | Synovial CD34+ |
| | | | | | sublining fibroblasts |
| COL5A3 | Fibroblasts | TGFBR2 | M2 | PODN | Synovial CD34+ |
| | | | | | sublining fibroblasts |
| COLEC10 | Fibroblasts | TLR5 | M2 | PTGFR | Synovial CD34+ |
| | | | | | sublining fibroblasts |
| COLEC12 | Fibroblasts | TPST2 | M2 | RPL35A | Synovial CD34+ |
| | | | | | sublining fibroblasts |
| CPXM2 | Fibroblasts | ABI1 | Macropinocytosis | RPL37 | Synovial CD34+ |
| | | | Signaling | | sublining fibroblasts |
| CRABP2 | Fibroblasts | ARF6 | Macropinocytosis | SERPINF1 | Synovial CD34+ |
| | | | Signaling | | sublining fibroblasts |
| CXCL12 | Fibroblasts | ATM | Macropinocytosis | SFRP1 | Synovial CD34+ |
| | | | Signaling | | sublining fibroblasts |
| CYB5R2 | Fibroblasts | CDC42 | Macropinocytosis | SFRP2 | Synovial CD34+ |
| | | | Signaling | | sublining fibroblasts |
| CYBRD1 | Fibroblasts | CSF1 | Macropinocytosis | VCAN | Synovial CD34+ |
| | | | Signaling | | sublining fibroblasts |
| DCLK2 | Fibroblasts | CSF1R | Macropinocytosis | ASPN | Synovial DKK3+ |
| | | | Signaling | | sublininig fibroblasts |
| DDR2 | Fibroblasts | FGFR1 | Macropinocytosis | CADM1 | Synovial DKK3+ |
| | | | Signaling | | sublininig fibroblasts |
| DDX58 | Fibroblasts | FRS2 | Macropinocytosis | COL5A1 | Synovial DKK3+ |
| | | | Signaling | | sublininig fibroblasts |
| DDX60 | Fibroblasts | GAB1 | Macropinocytosis | COL8A2 | Synovial DKK3+ |
| | | | Signaling | | sublininig fibroblasts |
| DHX58 | Fibroblasts | GRB2 | Macropinocytosis | COMP | Synovial DKK3+ |
| | | | Signaling | | sublininig fibroblasts |
| DKK1 | Fibroblasts | HGF | Macropinocytosis | DKK3 | Synovial DKK3+ |
| | | | Signaling | | sublininig fibroblasts |
| DKK3 | Fibroblasts | HRAS | Macropinocytosis | DPT | Synovial DKK3+ |
| | | | Signaling | | sublininig fibroblasts |
| DLL4 | Fibroblasts | IRS1 | Macropinocytosis | EMP1 | Synovial DKK3+ |
| | | | Signaling | | sublininig fibroblasts |
| DMRTA1 | Fibroblasts | ITGB1 | Macropinocytosis | FMOD | Synovial DKK3+ |
| | | | Signaling | | sublininig fibroblasts |
| DRAM1 | Fibroblasts | ITGB2 | Macropinocytosis | PDGFRB | Synovial DKK3+ |
| | | | Signaling | | sublininig fibroblasts |
| DTX3L | Fibroblasts | ITGB5 | Macropinocytosis | PRELP | Synovial DKK3+ |
| | | | Signaling | | sublininig fibroblasts |
| DUSP14 | Fibroblasts | ITGB7 | Macropinocytosis | B2M | Synovial HLA-DRhi |
| | | | Signaling | | sublining fibroblasts |
| ECM1 | Fibroblasts | KRAS | Macropinocytosis | CIS | Synovial HLA-DRhi |
| | | | Signaling | | sublining fibroblasts |
| EGFL6 | Fibroblasts | MET | Macropinocytosis | CD74 | Synovial HLA-DRhi |
| | | | Signaling | | sublining fibroblasts |
| EID3 | Fibroblasts | NGF | Macropinocytosis | HLA-A | Synovial HLA-DRhi |
| | | | Signaling | | sublining fibroblasts |
| ELN | Fibroblasts | NRAS | Macropinocytosis | HLA-B | Synovial HLA-DRhi |
| | | | Signaling | | sublining fibroblasts |
| ELOVL2 | Fibroblasts | PAK1 | Macropinocytosis | HLA-DPA1 | Synovial HLA-DRhi |
| | | | Signaling | | sublining fibroblasts |
| EMILIN1 | Fibroblasts | PDGFC | Macropinocytosis | HLA-DPB1 | Synovial HLA-DRhi |
| | | | Signaling | | sublining fibroblasts |
| ENPP2 | Fibroblasts | PDGFD | Macropinocytosis | HLA-DRA | Synovial HLA-DRhi |
| | | | Signaling | | sublining fibroblasts |
| EVA1B | Fibroblasts | PIK3C3 | Macropinocytosis | IFI27 | Synovial HLA-DRhi |
| | | | Signaling | | sublining fibroblasts |
| F10 | Fibroblasts | PIK3C2A | Macropinocytosis | LGALS3BP | Synovial HLA-DRhi |
| | | | Signaling | | sublining fibroblasts |
| FAM162B | Fibroblasts | PIK3C2B | Macropinocytosis | STAT1 | Synovial HLA-DRhi |
| | | | Signaling | | sublining fibroblasts |
| FAM20A | Fibroblasts | PIK3CB | Macropinocytosis | BCAT1 | Synovial lining |
| | | | Signaling | | fibroblasts |
| FBLN1 | Fibroblasts | PIK3CD | Macropinocytosis | CD55 | Synovial lining |
| | | | Signaling | | fibroblasts |
| FBLN5 | Fibroblasts | PIK3CG | Macropinocytosis | CRTAC1 | Synovial lining |
| | | | Signaling | | fibroblasts |
| FBLN7 | Fibroblasts | PIK3R1 | Macropinocytosis | FAM49A | Synovial lining |
| | | | Signaling | | fibroblasts |
| FGF20 | Fibroblasts | PLCG1 | Macropinocytosis | FN1 | Synovial lining |
| | | | Signaling | | fibroblasts |
| FGF5 | Fibroblasts | PLCG2 | Macropinocytosis | HTRA1 | Synovial lining |
| | | | Signaling | | fibroblasts |
| FHL1 | Fibroblasts | PRKCA | Macropinocytosis | NTN4 | Synovial lining |
| | | | Signaling | | fibroblasts |
| FMN2 | Fibroblasts | PRKCB | Macropinocytosis | SMIM14 | Synovial lining |
| | | | Signaling | | fibroblasts |
| FOXF1 | Fibroblasts | PRKCD | Macropinocytosis | THBS4 | Synovial lining |
| | | | Signaling | | fibroblasts |
| FOXF2 | Fibroblasts | PRKCI | Macropinocytosis | TIMP3 | Synovial lining |
| | | | Signaling | | fibroblasts |
| FST | Fibroblasts | PRKD1 | Macropinocytosis | BATF3 | T Cells |
| | | | Signaling | | |
| FSTL1 | Fibroblasts | PRKD3 | Macropinocytosis | CRTAM | T Cells |
| | | | Signaling | | |
| GABBR2 | Fibroblasts | PTPN11 | Macropinocytosis | CXCL13 | T Cells |
| | | | Signaling | | |
| GAS1 | Fibroblasts | RAB34 | Macropinocytosis | HAVCR2 | T Cells |
| | | | Signaling | | |
| GBP3 | Fibroblasts | RAB5A | Macropinocytosis | IL10 | T Cells |
| | | | Signaling | | |
| GDF5 | Fibroblasts | RAC1 | Macropinocytosis | IL18R1 | T Cells |
| | | | Signaling | | |
| GFRA1 | Fibroblasts | RHOA | Macropinocytosis | IL27RA | T Cells |
| | | | Signaling | | |
| GLIS1 | Fibroblasts | RRAS | Macropinocytosis | TNFSF11 | T Cells |
| | | | Signaling | | |
| GLIS3 | Fibroblasts | AVPI1 | MHCII high dendritic | CFP | T Cells |
| | | | cells STA | | |
| GLT8D2 | Fibroblasts | BCL2A1 | MHCII high dendritic | LAMP3 | T Cells |
| | | | cells STA | | |
| GPAT2 | Fibroblasts | BTG2 | MHCII high dendritic | LCP2 | T Cells |
| | | | cells STA | | |
| GPR176 | Fibroblasts | C15orf48 | MHCII high dendritic | PTPN7 | T Cells |
| | | | cells STA | | |
| GRAMD3 | Fibroblasts | CCDC88A | MHCII high dendritic | BATF2 | T Cells |
| | | | cells STA | | |
| GREM1 | Fibroblasts | CCND3 | MHCII high dendritic | CD226 | T Cells |
| | | | cells STA | | |
| GREM2 | Fibroblasts | CD72 | MHCII high dendritic | CD4 | T Cells |
| | | | cells STA | | |
| GRIA3 | Fibroblasts | CD74 | MHCII high dendritic | IER3 | T Cells |
| | | | cells STA | | |
| GRIK2 | Fibroblasts | CFP | MHCII high dendritic | BCL11B | T Cells |
| | | | cells STA | | |
| GRIP2 | Fibroblasts | CORO1A | MHCII high dendritic | CD2 | T Cells |
| | | | cells STA | | |
| GRK5 | Fibroblasts | CSF2RB | MHCII high dendritic | CD247 | T Cells |
| | | | cells STA | | |
| GRP | Fibroblasts | CTSH | MHCII high dendritic | CD3D | T Cells |
| | | | cells STA | | |
| GUCY1A2 | Fibroblasts | CXCL16 | MHCII high dendritic | FYB | T Cells |
| | | | cells STA | | |
| HOXC11 | Fibroblasts | CYTIP | MHCII high dendritic | HCST | T Cells |
| | | | cells STA | | |
| HSD17B2 | Fibroblasts | DAPP1 | MHCII high dendritic | IL2RB | T Cells |
| | | | cells STA | | |
| HSPA2 | Fibroblasts | ETV3 | MHCII high dendritic | ITK | T Cells |
| | | | cells STA | | |
| HSPB3 | Fibroblasts | FAM129A | MHCII high dendritic | LCK | T Cells |
| | | | cells STA | | |
| IGFBP6 | Fibroblasts | FGR | MHCII high dendritic | PAG1 | T Cells |
| | | | cells STA | | |
| IL19 | Fibroblasts | GLIPR2 | MHCII high dendritic | PIM2 | T Cells |
| | | | cells STA | | |
| IQCD | Fibroblasts | GM2A | MHCII high dendritic | PVRIG | T Cells |
| | | | cells STA | | |
| KCNJ3 | Fibroblasts | GPR132 | MHCII high dendritic | SIT1 | T Cells |
| | | | cells STA | | |
| KCNMB2 | Fibroblasts | H2AFY | MHCII high dendritic | STK10 | T Cells |
| | | | cells STA | | |
| KCNQ5 | Fibroblasts | HLA-DMB | MHCII high dendritic | THEMIS | T Cells |
| | | | cells STA | | |
| KIAA1456 | Fibroblasts | IFNGR1 | MHCII high dendritic | TRAC | T Cells |
| | | | cells STA | | |
| KIRREL3 | Fibroblasts | IL2RG | MHCII high dendritic | TRBC1 | T Cells |
| | | | cells STA | | |
| KLF17 | Fibroblasts | ITGAX | MHCII high dendritic | AIRE | T Cells |
| | | | cells STA | | |
| KRTAP1-5 | Fibroblasts | JAK2 | MHCII high dendritic | AKNA | T Cells |
| | | | cells STA | | |
| KRTAP3-1 | Fibroblasts | LACTB | MHCII high dendritic | CAMK4 | T Cells |
| | | | cells STA | | |
| LICAM | Fibroblasts | LSR | MHCII high dendritic | CBL | T Cells |
| | | | cells STA | | |
| LAYN | Fibroblasts | MPC1 | MHCII high dendritic | CCDC88B | T Cells |
| | | | cells STA | | |
| LMOD1 | Fibroblasts | MYD88 | MHCII high dendritic | CD160 | T Cells |
| | | | cells STA | | |
| LOXL2 | Fibroblasts | PAK1 | MHCII high dendritic | CD244 | T Cells |
| | | | cells STA | | |
| LOXL4 | Fibroblasts | PLSCR1 | MHCII high dendritic | CD28 | T Cells |
| | | | cells STA | | |
| LRRN3 | Fibroblasts | PSMB8 | MHCII high dendritic | CD300A | T Cells |
| | | | cells STA | | |
| LUM | Fibroblasts | PSMB9 | MHCII high dendritic | CD3E | T Cells |
| | | | cells STA | | |
| LY6K | Fibroblasts | PSME1 | MHCII high dendritic | CD3G | T Cells |
| | | | cells STA | | |
| LYNX1 | Fibroblasts | PTPRC | MHCII high dendritic | CD5 | T Cells |
| | | | cells STA | | |
| MAMLD1 | Fibroblasts | RGS1 | MHCII high dendritic | CD6 | T Cells |
| | | | cells STA | | |
| MAP3K7CL | Fibroblasts | SLAMF7 | MHCII high dendritic | CEACAM1 | T Cells |
| | | | cells STA | | |
| MARCH4 | Fibroblasts | SYNGR2 | MHCII high dendritic | CNR2 | T Cells |
| | | | cells STA | | |
| MASP1 | Fibroblasts | TAP1 | MHCII high dendritic | DNMT1 | T Cells |
| | | | cells STA | | |
| MGARP | Fibroblasts | TMEM123 | MHCII high dendritic | ETS1 | T Cells |
| | | | cells STA | | |
| MGLL | Fibroblasts | UVRAG | MHCII high dendritic | GFI1 | T Cells |
| | | | cells STA | | |
| MICU3 | Fibroblasts | VASP | MHCII high dendritic | GPR171 | T Cells |
| | | | cells STA | | |
| MKX | Fibroblasts | VRK1 | MHCII high dendritic | GPR65 | T Cells |
| | | | cells STA | | |
| MMP10 | Fibroblasts | YWHAH | MHCII high dendritic | HELLS | T Cells |
| | | | cells STA | | |
| MOXD1 | Fibroblasts | ATP2B1 | MHCII interstitial | HIVEP3 | T Cells |
| | | | macrophages STA | | |
| MPP4 | Fibroblasts | CD74 | MHCII interstitial | HLX | T Cells |
| | | | macrophages STA | | |
| MRC2 | Fibroblasts | CLEC12A | MHCII interstitial | HSH2D | T Cells |
| | | | macrophages STA | | |
| MRGPRF | Fibroblasts | DOK2 | MHCII interstitial | IFNG | T Cells |
| | | | macrophages STA | | |
| MRVI1 | Fibroblasts | GAS7 | MHCII interstitial | IL12RB2 | T Cells |
| | | | macrophages STA | | |
| MXRA5 | Fibroblasts | IRF2BP2 | MHCII interstitial | IL17A | T Cells |
| | | | macrophages STA | | |
| MXRA8 | Fibroblasts | JUN | MHCII interstitial | IL17F | T Cells |
| | | | macrophages STA | | |
| MYL9 | Fibroblasts | LIFR | MHCII interstitial | IL18RAP | T Cells |
| | | | macrophages STA | | |
| MYPN | Fibroblasts | MAF | MHCII interstitial | IL2 | T Cells |
| | | | macrophages STA | | |
| NBPF3 | Fibroblasts | MAFB | MHCII interstitial | IL21 | T Cells |
| | | | macrophages STA | | |
| NEXN | Fibroblasts | MEF2C | MHCII interstitial | IL22 | T Cells |
| | | | macrophages STA | | |
| NFASC | Fibroblasts | MS4A7 | MHCII interstitial | IL23R | T Cells |
| | | | macrophages STA | | |
| NFKBIZ | Fibroblasts | MTSS1 | MHCII interstitial | IL26 | T Cells |
| | | | macrophages STA | | |
| NID2 | Fibroblasts | RBPJ | MHCII interstitial | IL5 | T Cells |
| | | | macrophages STA | | |
| NKAPL | Fibroblasts | RTN4 | MHCII interstitial | IL9 | T Cells |
| | | | macrophages STA | | |
| NLRP10 | Fibroblasts | SERINC3 | MHCII interstitial | KLRB1 | T Cells |
| | | | macrophages STA | | |
| NRXN3 | Fibroblasts | TXNIP | MHCII interstitial | LAT | T Cells |
| | | | macrophages STA | | |
| NTF3 | Fibroblasts | WNK1 | MHCII interstitial | LEF1 | T Cells |
| | | | macrophages STA | | |
| NXPH3 | Fibroblasts | WWP1 | MHCII interstitial | MAL | T Cells |
| | | | macrophages STA | | |
| OLFML3 | Fibroblasts | CLEC10A | MHCII interstitial | NCKAP1L | T Cells |
| | | | macrophages steady- | | |
| | | | state | | |
| OXTR | Fibroblasts | CORO1A | MHCII interstitial | NCR3 | T Cells |
| | | | macrophages steady- | | |
| | | | state | | |
| P3H3 | Fibroblasts | GM2A | MHCII interstitial | NKAP | T Cells |
| | | | macrophages steady- | | |
| | | | state | | |
| P4HA3 | Fibroblasts | HLA-DMA | MHCII interstitial | NLRC3 | T Cells |
| | | | macrophages steady- | | |
| | | | state | | |
| PAMR1 | Fibroblasts | HLA-DQB1 | MHCII interstitial | NPY1R | T Cells |
| | | | macrophages steady- | | |
| | | | state | | |
| PAX3 | Fibroblasts | LSP1 | MHCII interstitial | PSTPIP1 | T Cells |
| | | | macrophages steady- | | |
| | | | state | | |
| PCDHGA3 | Fibroblasts | CLEC4A | MHCII interstitial | PTCRA | T Cells |
| | | | macrophages steady- | | |
| | | | state | | |
| PCDHGB5 | Fibroblasts | CD74 | MHCII interstitial | SEMA7A | T Cells |
| | | | macrophages steady- | | |
| | | | state | | |
| PCOLCE | Fibroblasts | ADAMDEC1 | Monos/Macs | SH2D2A | T Cells |
| PDE5A | Fibroblasts | ADGRE2 | Monos/Macs | SHCBP1 | T Cells |
| PDE8B | Fibroblasts | APOBEC3G | Monos/Macs | SIRPG | T Cells |
| PDGFRA | Fibroblasts | CCL8 | Monos/Macs | SKAP1 | T Cells |
| PDGFRL | Fibroblasts | CFP | Monos/Macs | SPN | T Cells |
| PDZRN3 | Fibroblasts | CLEC10A | Monos/Macs | TBX21 | T Cells |
| PKD2 | Fibroblasts | CSF2RB | Monos/Macs | TCF7 | T Cells |
| PKIG | Fibroblasts | CXCL10 | Monos/Macs | TEC | T Cells |
| PLA2R1 | Fibroblasts | CXCL9 | Monos/Macs | TESPA1 | T Cells |
| PLCXD3 | Fibroblasts | FCGR2B | Monos/Macs | TIAM1 | T Cells |
| PLEKHA4 | Fibroblasts | HVCN1 | Monos/Macs | TMIGD2 | T Cells |
| PLOD2 | Fibroblasts | IDO1 | Monos/Macs | TNFRSF18 | T Cells |
| PLPP4 | Fibroblasts | IL10RA | Monos/Macs | TOX | T Cells |
| PLPPR4 | Fibroblasts | IL18BP | Monos/Macs | TRAT1 | T Cells |
| PLXDC1 | Fibroblasts | LAMP3 | Monos/Macs | TRAV12-2 | T Cells |
| PODN | Fibroblasts | LCP2 | Monos/Macs | TRAV6 | T Cells |
| PRELP | Fibroblasts | MPEG1 | Monos/Macs | TRAV8-3 | T Cells |
| PRKCDBP | Fibroblasts | PECAM1 | Monos/Macs | TRBV27 | T Cells |
| PRKG1 | Fibroblasts | PLA2G2D | Monos/Macs | TXK | T Cells |
| PRR16 | Fibroblasts | PTPN7 | Monos/Macs | UBASH3A | T Cells |
| PRRX1 | Fibroblasts | PYHIN1 | Monos/Macs | UBASH3B | T Cells |
| PRRX2 | Fibroblasts | TNFAIP3 | Monos/Macs | ZBTB32 | T Cells |
| PSG2 | Fibroblasts | TNFAIP8L2 | Monos/Macs | ZEB1 | T Cells |
| PSG5 | Fibroblasts | TNFRSF1B | Monos/Macs | ZNF101 | T Cells |
| PSG7 | Fibroblasts | UBD | Monos/Macs | ZNF683 | T Cells |
| PSG9 | Fibroblasts | ACE | Monos/Macs | ZNF831 | T Cells |
| PTGS2 | Fibroblasts | ADAM8 | Monos/Macs | EOMES | Tanergic |
| PTRF | Fibroblasts | ADGRE1 | Monos/Macs | HAVCR2 | Tanergic |
| RCN3 | Fibroblasts | APOBEC3B | Monos/Macs | NCR3 | Tanergic |
| RECK | Fibroblasts | APOBR | Monos/Macs | ZEB1 | Tanergic |
| RGMB | Fibroblasts | ART4 | Monos/Macs | CTLA4 | Tanergic |
| S100A4 | Fibroblasts | BATF2 | Monos/Macs | GNG7 | Tec Kinase Signaling |
| SCN9A | Fibroblasts | C1QA | Monos/Macs | ITGA4 | Tec Kinase Signaling |
| SEMA3A | Fibroblasts | C1QC | Monos/Macs | ITK | Tec Kinase Signaling |
| SEMA5A | Fibroblasts | C2 | Monos/Macs | JAK2 | Tec Kinase Signaling |
| SEMA6D | Fibroblasts | C4BPA | Monos/Macs | LCK | Tec Kinase Signaling |
| SGCD | Fibroblasts | C4BPB | Monos/Macs | PLCG2 | Tec Kinase Signaling |
| SH3PXD2A | Fibroblasts | C5 | Monos/Macs | PRKCB | Tec Kinase Signaling |
| SHOX | Fibroblasts | C6 | Monos/Macs | RELA | Tec Kinase Signaling |
| SLC15A3 | Fibroblasts | C8a | Monos/Macs | RHOH | Tec Kinase Signaling |
| SLC16A2 | Fibroblasts | C9 | Monos/Macs | STAT2 | Tec Kinase Signaling |
| SLC22A4 | Fibroblasts | CCL17 | Monos/Macs | STAT5B | Tec Kinase Signaling |
| SLC35G2 | Fibroblasts | CCL18 | Monos/Macs | TNFSF10 | Tec Kinase Signaling |
| SLC38A11 | Fibroblasts | CCL22 | Monos/Macs | BCL6 | Tfh |
| SLC8A1 | Fibroblasts | CCL28 | Monos/Macs | ICOS | Tfh |
| SLIT2 | Fibroblasts | CCL5 | Monos/Macs | ASCL2 | Tfh |
| SMIM2 | Fibroblasts | CCL7 | Monos/Macs | PDCD1 | Tfh |
| SP100 | Fibroblasts | CD14 | Monos/Macs | TNFSF4 | Tfh |
| SPHKAP | Fibroblasts | CD163 | Monos/Macs | CD274 | Th1 Pathway |
| SSC5D | Fibroblasts | CD209 | Monos/Macs | CD8A | Th1 Pathway |
| SSTR1 | Fibroblasts | CD226 | Monos/Macs | CXCR3 | Th1 Pathway |
| STC1 | Fibroblasts | CD4 | Monos/Macs | HAVCR2 | Th1 Pathway |
| STXBP6 | Fibroblasts | CD5L | Monos/Macs | HLA-DPA1 | Th1 Pathway |
| SULF1 | Fibroblasts | CD80 | Monos/Macs | HLA-DPB1 | Th1 Pathway |
| SUSD5 | Fibroblasts | CD86 | Monos/Macs | HLA-DQB2 | Th1 Pathway |
| SVEP1 | Fibroblasts | CEACAM4 | Monos/Macs | ICAM1 | Th1 Pathway |
| SYNC | Fibroblasts | CFB | Monos/Macs | IL10RA | Th1 Pathway |
| SYT14 | Fibroblasts | CFD | Monos/Macs | ITGB2 | Th1 Pathway |
| TBX15 | Fibroblasts | CHI3L1 | Monos/Macs | LGALS9 | Th1 Pathway |
| TBX3 | Fibroblasts | CHIT1 | Monos/Macs | NFATC1 | Th1 Pathway |
| THBS2 | Fibroblasts | CLEC12A | Monos/Macs | NFATC2 | Th1 Pathway |
| THSD4 | Fibroblasts | CLEC12B | Monos/Macs | NFATC3 | Th1 Pathway |
| TIMP3 | Fibroblasts | CLEC16A | Monos/Macs | NFATC4 | Th1 Pathway |
| TMEM119 | Fibroblasts | CLEC1A | Monos/Macs | GATA3 | Th1 Pathway |
| TMEM158 | Fibroblasts | CLEC4C | Monos/Macs | IL10 | Th1 Pathway |
| TMEM200A | Fibroblasts | CLEC4D | Monos/Macs | IRF1 | Th1 Pathway |
| TMEM204 | Fibroblasts | CLEC4E | Monos/Macs | MIR29C | Th1 Pathway |
| TMEM47 | Fibroblasts | CLEC5A | Monos/Macs | MIR29B2 | Th1 Pathway |
| TNFRSF19 | Fibroblasts | CLEC7A | Monos/Macs | MIR29B1 | Th1 Pathway |
| TPM2 | Fibroblasts | CSF1R | Monos/Macs | MIR29A | Th1 Pathway |
| TRHDE | Fibroblasts | CSF2RA | Monos/Macs | APH1A | Th1 Pathway |
| TRIM22 | Fibroblasts | CXCL1 | Monos/Macs | APH1B | Th1 Pathway |
| TRPC4 | Fibroblasts | CXCL11 | Monos/Macs | ATM | Th1 Pathway |
| UACA | Fibroblasts | CXCL8 | Monos/Macs | CCR5 | Th1 Pathway |
| UBL4B | Fibroblasts | CYBB | Monos/Macs | CD4 | Th1 Pathway |
| USP53 | Fibroblasts | EBI3 | Monos/Macs | CD40 | Th1 Pathway |
| VATIL | Fibroblasts | F12 | Monos/Macs | CD80 | Th1 Pathway |
| VEGFC | Fibroblasts | FCGR1A | Monos/Macs | CD86 | Th1 Pathway |
| VIM | Fibroblasts | FCGR1B | Monos/Macs | CD3D | Th1 Pathway |
| WNT5A | Fibroblasts | FCGR2A | Monos/Macs | DLL1 | Th1 Pathway |
| WNT5B | Fibroblasts | FCGR2C | Monos/Macs | FGFR1 | Th1 Pathway |
| ZCCHC24 | Fibroblasts | FCGR3B | Monos/Macs | FRS2 | Th1 Pathway |
| ZCCHC5 | Fibroblasts | FFAR2 | Monos/Macs | GAB1 | Th1 Pathway |
| ZFPM2 | Fibroblasts | FGL2 | Monos/Macs | GRB2 | Th1 Pathway |
| ZNF486 | Fibroblasts | FPR2 | Monos/Macs | HLA-A | Th1 Pathway |
| ZP4 | Fibroblasts | FUT7 | Monos/Macs | HLA-B | Th1 Pathway |
| ACTR2 | fMLP Signaling in | HAVCR2 | Monos/Macs | HLA-DMA | Th1 Pathway |
| | Neutrophils | | | | |
| ARPC4 | fMLP Signaling in | HMMR | Monos/Macs | HLA-DMB | Th1 Pathway |
| | Neutrophils | | | | |
| ARPC5 | fMLP Signaling in | IER3 | Monos/Macs | HLA-DOA | Th1 Pathway |
| | Neutrophils | | | | |
| CALM1 | fMLP Signaling in | IGSF6 | Monos/Macs | HLA-DOB | Th1 Pathway |
| | Neutrophils | | | | |
| CALM2 | fMLP Signaling in | IL10 | Monos/Macs | HLA-DQB1 | Th1 Pathway |
| | Neutrophils | | | | |
| CALM3 | fMLP Signaling in | IL12A | Monos/Macs | HLA-DRA | Th1 Pathway |
| | Neutrophils | | | | |
| CDC42 | fMLP Signaling in | IL12B | Monos/Macs | HLA-DRB1 | Th1 Pathway |
| | Neutrophils | | | | |
| CYBB | fMLP Signaling in | IL15 | Monos/Macs | ICOS | Th1 Pathway |
| | Neutrophils | | | | |
| FGFR1 | fMLP Signaling in | IL18 | Monos/Macs | IFNGR1 | Th1 Pathway |
| | Neutrophils | | | | |
| FPR3 | fMLP Signaling in | IL1RAP | Monos/Macs | IL6 | Th1 Pathway |
| | Neutrophils | | | | |
| GNAI3 | fMLP Signaling in | IL20 | Monos/Macs | IL12RB1 | Th1 Pathway |
| | Neutrophils | | | | |
| GNAS | fMLP Signaling in | IL23A | Monos/Macs | IL18R1 | Th1 Pathway |
| | Neutrophils | | | | |
| GNB1 | fMLP Signaling in | IL27 | Monos/Macs | IL27RA | Th1 Pathway |
| | Neutrophils | | | | |
| GNB4 | fMLP Signaling in | IL2RA | Monos/Macs | IL6R | Th1 Pathway |
| | Neutrophils | | | | |
| GNG12 | fMLP Signaling in | IL31RA | Monos/Macs | IRS1 | Th1 Pathway |
| | Neutrophils | | | | |
| GRB2 | fMLP Signaling in | LGALS12 | Monos/Macs | JAK1 | Th1 Pathway |
| | Neutrophils | | | | |
| ITPR1 | fMLP Signaling in | LGALS4 | Monos/Macs | JAK2 | Th1 Pathway |
| | Neutrophils | | | | |
| KRAS | fMLP Signaling in | LGALS9 | Monos/Macs | MAP2K6 | Th1 Pathway |
| | Neutrophils | | | | |
| MAPK1 | fMLP Signaling in | LILRA1 | Monos/Macs | NFIL3 | Th1 Pathway |
| | Neutrophils | | | | |
| MAPK3 | fMLP Signaling in | LILRA2 | Monos/Macs | NOTCH1 | Th1 Pathway |
| | Neutrophils | | | | |
| NCF2 | fMLP Signaling in | LILRA5 | Monos/Macs | NOTCH4 | Th1 Pathway |
| | Neutrophils | | | | |
| NFKB2 | fMLP Signaling in | LILRA6 | Monos/Macs | PIK3C3 | Th1 Pathway |
| | Neutrophils | | | | |
| PIK3C2A | fMLP Signaling in | LILRB5 | Monos/Macs | PIK3C2A | Th1 Pathway |
| | Neutrophils | | | | |
| PIK3CB | fMLP Signaling in | LMNB1 | Monos/Macs | PIK3C2B | Th1 Pathway |
| | Neutrophils | | | | |
| PIK3CD | fMLP Signaling in | LTB4R | Monos/Macs | PIK3CB | Th1 Pathway |
| | Neutrophils | | | | |
| PIK3CG | fMLP Signaling in | LTF | Monos/Macs | PIK3CD | Th1 Pathway |
| | Neutrophils | | | | |
| PIK3R1 | fMLP Signaling in | LY86 | Monos/Macs | PIK3CG | Th1 Pathway |
| | Neutrophils | | | | |
| PIK3R4 | fMLP Signaling in | LYZ | Monos/Macs | PIK3R1 | Th1 Pathway |
| | Neutrophils | | | | |
| PLCB2 | fMLP Signaling in | MARCH1 | Monos/Macs | PSEN1 | Th1 Pathway |
| | Neutrophils | | | | |
| PPP3R1 | fMLP Signaling in | MARCO | Monos/Macs | PSEN2 | Th1 Pathway |
| | Neutrophils | | | | |
| PRKCB | fMLP Signaling in | MEGF10 | Monos/Macs | PSENEN | Th1 Pathway |
| | Neutrophils | | | | |
| PRKCI | fMLP Signaling in | MERTK | Monos/Macs | PTPN11 | Th1 Pathway |
| | Neutrophils | | | | |
| PRKD3 | fMLP Signaling in | MFGE8 | Monos/Macs | RUNX3 | Th1 Pathway |
| | Neutrophils | | | | |
| RAC1 | fMLP Signaling in | MRC1 | Monos/Macs | STAT1 | Th1 Pathway |
| | Neutrophils | | | | |
| LRMP | GC B cells | MS4A2 | Monos/Macs | STAT3 | Th1 Pathway |
| P2RY8 | GC B cells | MS4A4A | Monos/Macs | COMP | Tissue Repair/Tissue |
| | | | | | Destruction |
| IRF4 | GC B cells | MSR1 | Monos/Macs | SCRG1 | Tissue Repair/Tissue |
| | | | | | Destruction |
| KLHL6 | GC B cells | NECTIN2 | Monos/Macs | SPON1 | Tissue Repair/Tissue |
| | | | | | Destruction |
| FCRLA | GC B cells | NTSR1 | Monos/Macs | SCG2 | Tissue Repair/Tissue |
| | | | | | Destruction |
| RGS13 | GC B cells | OLR1 | Monos/Macs | EPYC | Tissue Repair/Tissue |
| | | | | | Destruction |
| CXCR5 | GC B cells | OTOF | Monos/Macs | GJB6 | Tissue Repair/Tissue |
| | | | | | Destruction |
| GCSAM | GC B cells | PDCDILG2 | Monos/Macs | SFRP2 | Tissue Repair/Tissue |
| | | | | | Destruction |
| LMNB1 | GC B cells | PILRA | Monos/Macs | ADAM12 | Tissue Repair/Tissue |
| | | | | | Destruction |
| AICDA | GC B cells | PLA2G5 | Monos/Macs | TUBB2B | Tissue Repair/Tissue |
| | | | | | Destruction |
| IRF4 | GC B cells | RNASE6 | Monos/Macs | F5 | Tissue Repair/Tissue |
| | | | | | Destruction |
| SELL | Granulocytes | S100A8 | Monos/Macs | LRRC15 | Tissue Repair/Tissue |
| | | | | | Destruction |
| CXCR3 | Granulocytes | S100A9 | Monos/Macs | PXDN | Tissue Repair/Tissue |
| | | | | | Destruction |
| IL16 | Granulocytes | S1PR5 | Monos/Macs | PI15 | Tissue Repair/Tissue |
| | | | | | Destruction |
| CSF2RB | Granulocytes | SCARB1 | Monos/Macs | CEMIP | Tissue Repair/Tissue |
| | | | | | Destruction |
| FCGR2B | Granulocytes | SCARF2 | Monos/Macs | RGS4 | Tissue Repair/Tissue |
| | | | | | Destruction |
| PECAM1 | Granulocytes | SECTM1 | Monos/Macs | TNC | Tissue Repair/Tissue |
| | | | | | Destruction |
| CEACAM4 | Granulocytes | SERPINB8 | Monos/Macs | COL11A1 | Tissue Repair/Tissue |
| | | | | | Destruction |
| CSF2RA | Granulocytes | SERPINB9 | Monos/Macs | ACLY | TNF Induced |
| CYBB | Granulocytes | SERPING1 | Monos/Macs | GRK3 | TNF Induced |
| FPR2 | Granulocytes | SIGLEC1 | Monos/Macs | AK3 | TNF Induced |
| FUT7 | Granulocytes | SIGLEC5 | Monos/Macs | AKAP10 | TNF Induced |
| LTB4R | Granulocytes | SIGLEC7 | Monos/Macs | AMPD3 | TNF Induced |
| MS4A2 | Granulocytes | SLC11A1 | Monos/Macs | APOL3 | TNF Induced |
| CD48 | Granulocytes | SLITRK4 | Monos/Macs | ARSE | TNF Induced |
| ADGRG5 | Granulocytes | SMPDL3B | Monos/Macs | B4GALT5 | TNF Induced |
| CXCL5 | Granulocytes | SPIC | Monos/Macs | NKX3-2 | TNF Induced |
| FCAR | Granulocytes | SRGN | Monos/Macs | BCL2A1 | TNF Induced |
| FCER2 | Granulocytes | STAB2 | Monos/Macs | BHMT | TNF Induced |
| IL5RA | Granulocytes | STAP2 | Monos/Macs | BIRC3 | TNF Induced |
| MMP25 | Granulocytes | TEK | Monos/Macs | CALD1 | TNF Induced |
| OR1J2 | Granulocytes | TGM2 | Monos/Macs | CASP1 | TNF Induced |
| PRG2 | Granulocytes | TIMD4 | Monos/Macs | CASP10 | TNF Induced |
| ADAM28 | HBEGF proinflammatory | TLR2 | Monos/Macs | CD37 | TNF Induced |
| | macrophages | | | | |
| ADAMDEC1 | HBEGF proinflammatory | TLR5 | Monos/Macs | CD38 | TNF Induced |
| | macrophages | | | | |
| B4GALT1 | HBEGF proinflammatory | TLR8 | Monos/Macs | CD83 | TNF Induced |
| | macrophages | | | | |
| BIRC3 | HBEGF proinflammatory | TNF | Monos/Macs | CDKN3 | TNF Induced |
| | macrophages | | | | |
| CXCL2 | HBEGF proinflammatory | TNIP3 | Monos/Macs | CKB | TNF Induced |
| | macrophages | | | | |
| CXCL3 | HBEGF proinflammatory | TULP1 | Monos/Macs | CR2 | TNF Induced |
| | macrophages | | | | |
| DNAJB1 | HBEGF proinflammatory | VENTX | Monos/Macs | CTNND2 | TNF Induced |
| | macrophages | | | | |
| FCERIA | HBEGF proinflammatory | VSIG4 | Monos/Macs | CYP27B1 | TNF Induced |
| | macrophages | | | | |
| HNRNPU | HBEGF proinflammatory | VSTM1 | Monos/Macs | DAB2 | TNF Induced |
| | macrophages | | | | |
| HSP90AA1 | HBEGF proinflammatory | TNFSF14 | Myeloid Cells | ASAP1 | TNF Induced |
| | macrophages | | | | |
| HSPH1 | HBEGF proinflammatory | ADGRE5 | Myeloid Cells | BHLHE41 | TNF Induced |
| | macrophages | | | | |
| LDLR | HBEGF proinflammatory | AOAH | Myeloid Cells | ARID3A | TNF Induced |
| | macrophages | | | | |
| PTGER4 | HBEGF proinflammatory | BTK | Myeloid Cells | EBI3 | TNF Induced |
| | macrophages | | | | |
| RASSF5 | HBEGF proinflammatory | FYB | Myeloid Cells | EGR1 | TNF Induced |
| | macrophages | | | | |
| RHOB | HBEGF proinflammatory | SLAMF8 | Myeloid Cells | EGR2 | TNF Induced |
| | macrophages | | | | |
| SELK | HBEGF proinflammatory | ADGRE3 | Myeloid Cells | ADGRE2 | TNF Induced |
| | macrophages | | | | |
| SEMA4A | HBEGF proinflammatory | APOC1 | Myeloid Cells | EPB41 | TNF Induced |
| | macrophages | | | | |
| TNFAIP3 | HBEGF proinflammatory | BACH1 | Myeloid Cells | EREG | TNF Induced |
| | macrophages | | | | |
| USP36 | HBEGF proinflammatory | BMX | Myeloid Cells | ETAA1 | TNF Induced |
| | macrophages | | | | |
| TNK2 | IFN Beta Up | BST1 | Myeloid Cells | F3 | TNF Induced |
| ACLY | IFN Beta Up | CD101 | Myeloid Cells | FABP1 | TNF Induced |
| ACSL1 | IFN Beta Up | CD300LF | Myeloid Cells | ACSL1 | TNF Induced |
| ADAM19 | IFN Beta Up | CD33 | Myeloid Cells | FBXL2 | TNF Induced |
| ADAP2 | IFN Beta Up | CLEC2B | Myeloid Cells | FCER2 | TNF Induced |
| ADAR | IFN Beta Up | CSF3R | Myeloid Cells | FCGR2A | TNF Induced |
| ADGRE2 | IFN Beta Up | FGR | Myeloid Cells | LRRN3 | TNF Induced |
| ADM | IFN Beta Up | IFNL1 | Myeloid Cells | FLNA | TNF Induced |
| AFF3 | IFN Beta Up | IFNL2 | Myeloid Cells | GOS2 | TNF Induced |
| AGT | IFN Beta Up | IL1A | Myeloid Cells | GBP1 | TNF Induced |
| AIM2 | IFN Beta Up | IL1B | Myeloid Cells | GCH1 | TNF Induced |
| AKAP10 | IFN Beta Up | IL1F10 | Myeloid Cells | GJB2 | TNF Induced |
| ALOX12 | IFN Beta Up | IL1RN | Myeloid Cells | GLS | TNF Induced |
| ALOX5 | IFN Beta Up | IL26 | Myeloid Cells | GP1BA | TNF Induced |
| ANXA4 | IFN Beta Up | IL37 | Myeloid Cells | CXCL1 | TNF Induced |
| APOBEC3B | IFN Beta Up | ITGAM | Myeloid Cells | CXCL2 | TNF Induced |
| APOBEC3G | IFN Beta Up | ITGAX | Myeloid Cells | CXCL3 | TNF Induced |
| APOL3 | IFN Beta Up | LILRB1 | Myeloid Cells | HHEX | TNF Induced |
| ATF3 | IFN Beta Up | LILRB2 | Myeloid Cells | HCAR3 | TNF Induced |
| ATF5 | IFN Beta Up | LILRB3 | Myeloid Cells | HOMER2 | TNF Induced |
| ATM | IFN Beta Up | LILRB4 | Myeloid Cells | HP | TNF Induced |
| ATP13A1 | IFN Beta Up | MNDA | Myeloid Cells | MGLL | TNF Induced |
| B4GAT1 | IFN Beta Up | PIK3AP1 | Myeloid Cells | BRCA2 | TNF Induced |
| BAG1 | IFN Beta Up | PRAM1 | Myeloid Cells | ICAM1 | TNF Induced |
| BAK1 | IFN Beta Up | PSTPIP1 | Myeloid Cells | IKBKG | TNF Induced |
| BARD1 | IFN Beta Up | SIGLEC10 | Myeloid Cells | IL16 | TNF Induced |
| BCL11A | IFN Beta Up | SKAP2 | Myeloid Cells | IL18 | TNF Induced |
| BCL7B | IFN Beta Up | SLPI | Myeloid Cells | IL1A | TNF Induced |
| BGN | IFN Beta Up | SPI1 | Myeloid Cells | IL1B | TNF Induced |
| BLNK | IFN Beta Up | TNFSF13B | Myeloid Cells | IL1RN | TNF Induced |
| BLVRA | IFN Beta Up | TREM1 | Myeloid Cells | IL6 | TNF Induced |
| BLZF1 | IFN Beta Up | TREML2 | Myeloid Cells | CXCL8 | TNF Induced |
| BRCA1 | IFN Beta Up | TREML4 | Myeloid Cells | PATJ | TNF Induced |
| BRCA2 | IFN Beta Up | TWIST1 | Myeloid Cells | IDO1 | TNF Induced |
| BST2 | IFN Beta Up | TWIST2 | Myeloid Cells | INHBA | TNF Induced |
| BUB1 | IFN Beta Up | HSH2D | Neg reg T cells | INSIG1 | TNF Induced |
| C3AR1 | IFN Beta Up | PAG1 | Neg reg T cells | ITGA6 | TNF Induced |
| CACNA1A | IFN Beta Up | SIT1 | Neg reg T cells | PDE4DIP | TNF Induced |
| CAD | IFN Beta Up | STK10 | Neg reg T cells | KITLG | TNF Induced |
| CALD1 | IFN Beta Up | PVRIG | Neg reg T cells | KLF1 | TNF Induced |
| CAMK2A | IFN Beta Up | ZEB1 | Neg reg T cells | KMO | TNF Induced |
| CAPN2 | IFN Beta Up | SPN | Neg reg T cells | LGALS3BP | TNF Induced |
| CASP1 | IFN Beta Up | HAVCR2 | Neg reg T cells | GMIP | TNF Induced |
| CASP10 | IFN Beta Up | CD274 | Neg reg T cells | MARCKS | TNF Induced |
| CASP5 | IFN Beta Up | IL27RA | Neutrophils | NFKBIZ | TNF Induced |
| CBR1 | IFN Beta Up | CFP | Neutrophils | MAP3K4 | TNF Induced |
| CCL13 | IFN Beta Up | C6 | Neutrophils | MMP19 | TNF Induced |
| CCL4 | IFN Beta Up | FCGR3A | Neutrophils | MN1 | TNF Induced |
| CCL7 | IFN Beta Up | LILRA2 | Neutrophils | MRPS15 | TNF Induced |
| CCL8 | IFN Beta Up | LILRA5 | Neutrophils | MSC | TNF Induced |
| CCNA1 | IFN Beta Up | RNASE6 | Neutrophils | IFI44 | TNF Induced |
| CCND2 | IFN Beta Up | SIGLEC14 | Neutrophils | MTF1 | TNF Induced |
| CCR1 | IFN Beta Up | CD83 | Neutrophils | MX1 | TNF Induced |
| CCR5 | IFN Beta Up | GPR84 | Neutrophils | NELL2 | TNF Induced |
| CCRL2 | IFN Beta Up | SAMSN1 | Neutrophils | NFKB1 | TNF Induced |
| CD163 | IFN Beta Up | SELL | Neutrophils | NFKB2 | TNF Induced |
| CD164 | IFN Beta Up | ADGRG3 | Neutrophils | NFKBIA | TNF Induced |
| CD2AP | IFN Beta Up | BTNL3 | Neutrophils | NR3C1 | TNF Induced |
| CD38 | IFN Beta Up | C3 | Neutrophils | OAS3 | TNF Induced |
| CD4 | IFN Beta Up | CD177 | Neutrophils | NAMPT | TNF Induced |
| CD59 | IFN Beta Up | CD300A | Neutrophils | PIAS4 | TNF Induced |
| CD69 | IFN Beta Up | CEACAM1 | Neutrophils | PLAUR | TNF Induced |
| CD72 | IFN Beta Up | CEACAM3 | Neutrophils | PTGES | TNF Induced |
| CD86 | IFN Beta Up | CLEC4A | Neutrophils | PTGS2 | TNF Induced |
| CDK17 | IFN Beta Up | CXCR2 | Neutrophils | RELB | TNF Induced |
| CDKN1A | IFN Beta Up | DOK2 | Neutrophils | RPGR | TNF Induced |
| CENPA | IFN Beta Up | FPR1 | Neutrophils | RPS9 | TNF Induced |
| CENPE | IFN Beta Up | FUT4 | Neutrophils | CCL20 | TNF Induced |
| CFB | IFN Beta Up | HLX | Neutrophils | CCL23 | TNF Induced |
| CFLAR | IFN Beta Up | IL17RA | Neutrophils | SDC4 | TNF Induced |
| CH25H | IFN Beta Up | MMP9 | Neutrophils | SERPIND1 | TNF Induced |
| CHI3L2 | IFN Beta Up | PGLYRP1 | Neutrophils | SFRP1 | TNF Induced |
| CHKA | IFN Beta Up | PI3 | Neutrophils | SH3BP5 | TNF Induced |
| CISH | IFN Beta Up | RNASE7 | Neutrophils | SLAMF1 | TNF Induced |
| CKB | IFN Beta Up | S1PR4 | Neutrophils | SLC30A4 | TNF Induced |
| CMAHP | IFN Beta Up | SERPINB1 | Neutrophils | SOD2 | TNF Induced |
| CNTN6 | IFN Beta Up | SIGLEC9 | Neutrophils | SPI1 | TNF Induced |
| CNTRL | IFN Beta Up | SIRPB1 | Neutrophils | SSPN | TNF Induced |
| COL3A1 | IFN Beta Up | IL3RA | pDC | STAT4 | TNF Induced |
| COX17 | IFN Beta Up | CLEC4C | pDC | PDPN | TNF Induced |
| CSF2RB | IFN Beta Up | NRP1 | pDC | TAF15 | TNF Induced |
| CTSL | IFN Beta Up | ACAD9 | Phagocytic | TAP2 | TNF Induced |
| | | | macrophages | | |
| CXCL10 | IFN Beta Up | ADAM12 | Phagocytic | TBX3 | TNF Induced |
| | | | macrophages | | |
| CXCL11 | IFN Beta Up | AHNAK2 | Phagocytic | TFF1 | TNF Induced |
| | | | macrophages | | |
| CXCL2 | IFN Beta Up | ANKH | Phagocytic | TNF | TNF Induced |
| | | | macrophages | | |
| CXCR2 | IFN Beta Up | AP3M1 | Phagocytic | TNFAIP2 | TNF Induced |
| | | | macrophages | | |
| CYBB | IFN Beta Up | ARMC9 | Phagocytic | TNFAIP3 | TNF Induced |
| | | | macrophages | | |
| CYP19A1 | IFN Beta Up | ASB1 | Phagocytic | TNFRSF11A | TNF Induced |
| | | | macrophages | | |
| CYP2J2 | IFN Beta Up | CALU | Phagocytic | TRAF1 | TNF Induced |
| | | | macrophages | | |
| DAB2 | IFN Beta Up | CSPG4 | Phagocytic | TSC22D1 | TNF Induced |
| | | | macrophages | | |
| DEFB1 | IFN Beta Up | DCBLD2 | Phagocytic | TYROBP | TNF Induced |
| | | | macrophages | | |
| DHFR | IFN Beta Up | DIP2C | Phagocytic | UBE2C | TNF Induced |
| | | | macrophages | | |
| DLL1 | IFN Beta Up | DNM1 | Phagocytic | VEGFA | TNF Induced |
| | | | macrophages | | |
| DMXL1 | IFN Beta Up | EMP1 | Phagocytic | WT1 | TNF Induced |
| | | | macrophages | | |
| DNMT1 | IFN Beta Up | EXTL2 | Phagocytic | FOXP3 | Treg |
| | | | macrophages | | |
| DRAP | IFN Beta Up | FAM134B | Phagocytic | IKZF2 | Treg |
| | | | macrophages | | |
| DSC2 | IFN Beta Up | FERMT2 | Phagocytic | | |
| | | | macrophages | | |
| DUSP5 | IFN Beta Up | FGD5 | Phagocytic | | |
| | | | macrophages | | |

### Example 6: Molecular Endotypes of RA Patient

### Synovium Sample

A method was carried out to characterize the molecular landscape of patients with rheumatoid arthritis (RA) by analyzing gene expression profiles from synovium samples. Synovium samples were collected from a patient population comprising patients with rheumatoid arthritis (RA) that are incomplete responders (IR) to DMARD treatment (RA DMARD IR patients) treated with TNFi.

Full transcriptomic RNA sequencing was carried out on synovium samples collected from a patient population. The RNA sequencing assay involved 46 RA DMARD IR patients treated with TNFi.

In brief, synovium samples were collected. After removal of ribosomal RNA and globin transcripts with the Ribo-Zero Globin Removal kit (Illumina), stranded libraries are prepared with the TruSeq Library prep kit (Illumina) and hybridized to a flow cell for sequencing with the Illumina HiSeq platform. Raw RNAseq output counts are log 2 normalized using the R DESeq2 package. The top 5,000 row variance (top5k rowVar) genes determined using standard deviation between samples were retained for further analysis.

The top 5,000 row variance genes were analyzed by a suite of gene expression technologies, including Multiscale Embedded Gene Co-expression Network Analysis (MEGENA) to generate gene coexpression modules which were functionally annotated and correlated to various demographic traits, clinical features, and laboratory assays.

In brief, the MEGENA R package was used to generate a gene coexpression network by inputting the top5k rowVar genes. MEGENA multi-scale clustering analysis (MCA) formed lineages of gene modules followed by identification of densely intraconnected hub genes using multi-scale hub analysis (MHA). Modules were assigned “lineage” names based on their multiscale pedigree from the root MEGENA module. The prcomp package was utilized to perform singular value decomposition and calculate MEGENA module eigengenes (MEs), equivalent to the first principal component calculated amongst the variance of a given MEGENA module. MEGENA MEs were correlated to the numerically encoded sample traits.

A heatmap was generated using ComplexHeatmap visualizing the top 40 sample trait correlations to the MEGENA modules that were significantly correlated to cohort (or cluster.) Module gene symbols were used to programmatically query the STRING database and calculate the percentage of genes within a given module predicted to have known protein-protein interactions (PPI) ranging from 0 to 100%.

A gene set variation analysis (GSVA) (GSVA (V1.25.0) R software package) was carried out as a non-parametric, unsupervised method for estimating the variation of pre-defined gene sets over all MEGENA module log 2 gene expression values. Input genes were employed only if the interquartile range (IQR) of their expression across the samples was greater than 0. Enrichment scores (GSVA scores) were calculated non-parametrically using a Kolmogorov Smirnoff (KS)-like random walk statistic. The enrichment scores(ES) were the largest positive and negative random walk deviations from zero, respectively, for a particular sample amongst the module gene set. The GSVA scores were used as input for unsupervised stable k-means clustering, and five different disease phenotypes or clusters were identified (i.e., cluster 0, cluster 1, cluster 2, cluster 3, cluster 4). GSVA was performed using the significant MEGENA modules as gene signatures.

The MEs of the significant MEGENA modules were correlated to mean gene expression of a given module per patient and visualized using Complex heatmap. As shown in FIG. 12, columns of patients with RA that are TNF-IR were clustered using idealized k-means clustering on patient samples and three different disease phenotypes or clusters were identified (i.e., cluster 1, cluster 2, cluster 3). Also as shown in FIG. 12, rows correspond to various immune cell type and process modules (e.g., MEGENA modules: A, B, C, D, E, F, and G) used to identify the three patient subsets. In brief, the modules, can be associated to subsets with enriched treatment targets. Overall, stable k-means clustering of gene coexpression modules effectively segregated patient subsets into three different disease phenotypes which may be associated to different treatment targets and/or responsive to different treatments. Overall, FIG. 12 shows clinical response of RA DMARD-IR patients treated with TNFi. As shown in FIG. 12A, rows correspond to various EULAR response criteria (i.e., subject trait or patient trait), used to classify individual patients as non-responders, moderate responders, or high responders, depending on the extent of change and the level of disease activity reached. As shown in FIG. 12A, rows visualize in color (color choice arbitrarily selected to ease visualization) whether the EULAR response criteria (e.g., patient traits or subject traits: patient is female (pt.is.female), patient ancestry (pt.anc.), patient ancestry African (pt.anc.AA), patient ancestry Asian (pt.anc.AsA), patient ancestry Caribean (pt.anc.Carib), patient ancestry European (pt.anc.EA), patient treatment certolizumab pegol (CZP) (pt.treatment.CZP), patient treatment ETC (pt.treatment.ETC), patient non responder (patient.responder.non), patient moderate responder (patient.responder.mod), patient high responder (patient.responder.high), patient pauci-immune designation (pt.histo.pauci.immune), patient biopsy from wrist (pt.biop.wrist), patient biopsy from knee (pt.biop.knee), patient indication of RA erosion (pt.ra.erosion), patient pathology myeloid (pt.patho.myeloid), patient pathology lymphoid (pt.patho.lymphoid), patient pathology fibroid (pt.patho.fibroid), etc.) was met. Gradations of color intensity, from low color intensity to high color intensity, indicates degree to which criteria was met (e.g., continuous values: patient age (pt.age), patient inflammatory score (pt.score.inf), patient DAS28 score (pt.score.das28), patient change in DAS28 score after treatment (pt.score.das28.delta), patient HAQ score disease index (pt.score.haq.di), patient disease duration (pt.disease.duration), number of swollen joints (pt.count.joint.swollen), number of tender joints (pt.count.joint.tender), CRP C-reactive protein level (pt.CRP), patient erythrocyte sedimentation rate (ESR pt.ESR), patient rheumatoid factor (pt.RF), patient ACPA level (pt.ACPA), patient blood sample RNA concentration (pt.RNA.concentration), patient blood sample total RNA volume (pt.RNA.volume), patient blood sample total RNA yield (pt.RNA.yield), etc.). Also as shown in FIG. 12A, no color intensity shows DMARD-IR patients did not respond to TNFi treatment, low color intensity shows DMARD-IR patients were moderate responders to TNFi treatment, high color intensity shows DMARD-IR patients were high responders to TNFi treatment. Overall, the heatmap in FIG. 12A (see dark green at the bottom) shows high responders to TNFi identified in Cluster 2, as compared to low responders to TNFi identified in Cluster 3.

As shown in FIG. 12B, the heatmap color intensity represents the enrichment of gene signature, with bright red indicating increased enrichment and blue decreased enrichment, and white representing the intermediate levels. In brief, a high GSVA score (bright red) indicates a high response to TNFi therapy. Also as shown in both FIG. 12B and FIG. 12C, rows correspond to various immune cell type and process modules (e.g., MEGENA modules: A, B, C, D, E, F, and G) used to identify the patient subsets. The columns in FIG. 12C correspond to various clinical criteria. Overall, FIG. 12C details correlations between the various immune cell type and process modules (MEGENA modules) and the clinical criteria. As shown in FIG. 12C, rows visualize correlations in color, ranging from negative (blue, or 0) to positive (red, or 1), with white representing intermediate. Table 5 details the genes within the MEGENA modules.

TABLE 5
details the genes within the MEGENA modules

| Gene | Category | Gene | Category | Gene | Category | Gene | Category |
|---|---|---|---|---|---|---|---|
| MT-ATP6 | 2.5 | MED18 | 2.7 | FIBCD1 | 3.17 | RGS16 | 2.7.36 |
| MT-ND2 | 2.5 | PYURF | 2.7 | SNAI3 | 3.17 | SIGIRR | 2.7.36 |
| MT-ND1 | 2.5 | SIGIRR | 2.7 | ZNF625 | 3.17 | LIPK | 2.7.36 |
| MT-CO1 | 2.5 | TPSB2 | 2.7 | NRK | 3.17 | FZD8 | 2.7.36 |
| MT-CYB | 2.5 | LIPK | 2.7 | PPEF1 | 3.17 | WDR83OS | 2.7.36 |
| UBC | 2.5 | CBX4 | 2.7 | TRPC5 | 3.17 | CPTP | 2.7.36 |
| ACTB | 2.5 | FZD8 | 2.7 | VSIG1 | 3.17 | FAM180A | 2.7.36 |
| ZFP36L1 | 2.5 | OR10A2 | 2.7 | HS6ST2 | 3.17 | DPM3 | 2.7.36 |
| ACTG1 | 2.5 | WDR83OS | 2.7 | SRY | 3.17 | KRTAP6-1 | 2.7.36 |
| PTMA | 2.5 | CPTP | 2.7 | IL1RAPL1 | 3.17 | IL17A | 2.7.36 |
| COL3A1 | 2.5 | PCEDIA | 2.7 | BEND2 | 3.17 | CCL1 | 2.7.36 |
| RPL8 | 2.5 | HOXC12 | 2.7 | HTR2C | 3.17 | OR6T1 | 2.7.36 |
| ANP32B | 2.5 | FAM180A | 2.7 | IL1RAPL2 | 3.17 | NMB | 2.7.36 |
| COL6A1 | 2.5 | JPH2 | 2.7 | ABHD1 | 3.17 | ATG4D | 2.7.36 |
| MT-ND5 | 2.5 | DPM3 | 2.7 | LPAR4 | 3.17 | ZGPAT | 2.7.36 |
| MT-ND4L | 2.5 | CLIP3 | 2.7 | CHIC1 | 3.17 | ZBTB4 | 2.7.37 |
| PMP22 | 2.5 | RPL10L | 2.7 | GAGE1 | 3.17 | SPTAN1 | 2.7.37 |
| ARF1 | 2.5 | LDOC1 | 2.7 | FAM9A | 3.17 | OS9 | 2.7.37 |
| VIM | 2.5 | KRTAP6-1 | 2.7 | SMIM10L2A | 3.17 | HDLBP | 2.7.37 |
| TXNIP | 2.5 | SAT2 | 2.7 | SPANXN5 | 3.17 | USP22 | 2.7.37 |
| CD63 | 2.5 | KDM4E | 2.7 | TNMD | 3.17 | WDR1 | 2.7.37 |
| PKM | 2.5 | AOC3 | 2.7 | RAB41 | 3.17 | UBA1 | 2.7.37 |
| PSAP | 2.5 | TRAPPC12 | 2.7 | ARSH | 3.17 | ZMIZ1 | 2.7.37 |
| C1R | 2.5 | ERICH5 | 2.7 | OR51T1 | 3.17 | HCFC1 | 2.7.37 |
| SERPING1 | 2.5 | CDC42EP2 | 2.7 | MTIF3 | 3.17 | TMEM259 | 2.7.37 |
| TMSB10 | 2.5 | DCTPP1 | 2.7 | CHRDL1 | 3.17 | WASF2 | 2.7.37 |
| TIMP2 | 2.5 | NBPF14 | 2.7 | CXorf58 | 3.17 | DAP | 2.7.37 |
| ALDOA | 2.5 | ABHD11 | 2.7 | TCF23 | 3.17 | IRAK1 | 2.7.37 |
| CTSB | 2.5 | SOAT2 | 2.7 | MAGEB4 | 3.17 | TUBB4B | 2.7.37 |
| DDX17 | 2.5 | IL17A | 2.7 | XAGE5 | 3.17 | H6PD | 2.7.37 |
| MT-CO2 | 2.5 | RNF113B | 2.7 | RANBP1 | 3.17 | CSF1 | 2.7.37 |
| EIF1 | 2.5 | TCEAL1 | 2.7 | BMP15 | 3.17 | PSMD8 | 2.7.37 |
| TMSB4X | 2.5 | FRAT2 | 2.7 | PPP1R3F | 3.17 | TPD52L2 | 2.7.37 |
| MSN | 2.5 | CCL1 | 2.7 | LUZP4 | 3.17 | LMF2 | 2.7.37 |
| GRN | 2.5 | OR6T1 | 2.7 | FAM47A | 3.17 | RABAC1 | 2.7.37 |
| BGN | 2.5 | NTSR2 | 2.7 | RNASE7 | 3.17 | NUCB1 | 2.7.37 |
| LUC7L3 | 2.5 | IFI35 | 2.7 | TBL1Y | 3.17 | CCND1 | 2.7.37 |
| SRRM2 | 2.5 | THRSP | 2.7 | GPR173 | 3.17 | IQSEC1 | 2.7.37 |
| FTL | 2.5 | ILK | 2.7 | SNX32 | 3.17 | ALKBH5 | 2.7.37 |
| FLNA | 2.5 | ASTL | 2.7 | ZCCHC18 | 3.17 | GM2A | 2.7.37 |
| DPYSL2 | 2.5 | NOL3 | 2.7 | PSG4 | 3.17 | NDST1 | 2.7.37 |
| EEF2 | 2.5 | NMB | 2.7 | OR1E2 | 3.17 | SMARCC2 | 2.7.37 |
| CIQC | 2.5 | CST9L | 2.7 | PIP5KL1 | 3.17 | CSNKIE | 2.7.37 |
| NOTCH2 | 2.5 | AGT | 2.7 | OR4K15 | 3.17 | OTUD5 | 2.7.37 |
| CSF1R | 2.5 | PRDM13 | 2.7 | MAGEA8 | 3.17 | LDB1 | 2.7.37 |
| CD74 | 2.5 | THTPA | 2.7 | FGF16 | 3.17 | RAD23A | 2.7.37 |
| PLTP | 2.5 | OR56A4 | 2.7 | MRGPRX3 | 3.17 | CHD4 | 2.7.37 |
| ISLR | 2.5 | DVL1 | 2.7 | PAGE3 | 3.17 | GTF2F1 | 2.7.37 |
| ATP2B4 | 2.5 | KRTAP4-3 | 2.7 | GGTLC1 | 3.17 | ARHGAP1 | 2.7.37 |
| CYB5R3 | 2.5 | CELA2B | 2.7 | KCNA5 | 3.12 | CLPTM1 | 2.7.37 |
| ZFP36L2 | 2.5 | PPP1RIA | 2.7 | MLNR | 3.17 | FOSL2 | 2.7.37 |
| MMP2 | 2.5 | KCNQ2 | 2.7 | PAGE1 | 3.12 | INPPL1 | 2.7.37 |
| OAZ1 | 2.5 | NPY | 2.7 | MAGEA4 | 3.17 | MAGED2 | 2.7.37 |
| NFIC | 2.5 | SMIM10 | 2.7 | KRTAP15-1 | 3.17 | CPSF1 | 2.7.37 |
| AHNAK | 2.5 | ATG4D | 2.7 | OR2Y1 | 3.17 | UBXN1 | 2.7.37 |
| CD81 | 2.5 | GOS2 | 2.7 | TNFRSF18 | 3.17 | NRBP2 | 2.7.37 |
| GNAI2 | 2.5 | SCGB1D4 | 2.7 | SPANXN3 | 3.17 | ITM2C | 2.7.37 |
| PCBP1 | 2.5 | CAMK2N1 | 2.7 | TRIM43 | 3.17 | COL4A2 | 2.7.37 |
| CTSD | 2.5 | GAL3ST1 | 2.7 | WDR38 | 3.17 | CDK16 | 2.7.37 |
| PLEKHM2 | 2.5 | CER1 | 2.7 | EPHA2 | 3.12 | ZBTB47 | 2.7.37 |
| COL6A2 | 2.5 | HES6 | 2.7 | CT47B1 | 3.17 | CYC1 | 2.7.37 |
| NFIX | 2.5 | CACNA1H | 2.7 | OR9I1 | 3.12 | RPL10 | 2.7.37 |
| LGALS1 | 2.5 | CLEC14A | 2.7 | ASIP | 3.17 | MAP2K2 | 2.7.37 |
| COL1A2 | 2.5 | RNASE11 | 2.7 | BCKDHA | 3.17 | UBTD1 | 2.7.37 |
| DCN | 2.5 | ABHD8 | 2.7 | MTMR10 | 3.17 | JMJD8 | 2.7.37 |
| CTSA | 2.5 | ZGPAT | 2.7 | SPEF1 | 3.17 | PNPLA2 | 2.7.37 |
| HTRA1 | 2.5 | RFPL1 | 2.7 | GJA9 | 3.17 | OAF | 2.7.37 |
| COLEC12 | 2.5 | AMELX | 2.7 | TLR9 | 3.17 | TUBG2 | 2.7.37 |
| SPARC | 2.5 | FAM166B | 2.7 | IL17REL | 3.12 | OR4P4 | 2.7.37 |
| TACC1 | 2.5 | C3orf80 | 2.7 | SPDYE5 | 3.17 | ADAM15 | 2.7.37 |
| FCGR2A | 2.5 | TPTE | 2.7 | RAB40AL | 3.17 | SLC35C1 | 2.7.37 |
| FN1 | 2.5 | C16orf86 | 2.7 | ANGPTL4 | 3.17 | PRKCSH | 2.7.37 |
| JUND | 2.5 | PRSS58 | 2.7 | ARL6IP4 | 3.17 | AKT1 | 2.7.37 |
| EMP3 | 2.5 | ACOT1 | 2.7 | CSMD3 | 3.18 | F8A1 | 2.7.37 |
| IGFBP7 | 2.5 | ARHGEF18 | 2.7 | TTN | 3.18 | TMEM184B | 2.7.37 |
| PPDPF | 2.5 | FAM72D | 2.7 | CDH9 | 3.18 | NACC1 | 2.7.37 |
| COL1A1 | 2.5 | SPATA25 | 2.7 | SYCP1 | 3.18 | WBP1 | 2.7.37 |
| TMEM43 | 2.5 | SON | 2.8 | MGAT4C | 3.18 | MEOX1 | 2.7.37 |
| C1QB | 2.5 | KMT2E | 2.8 | RPS6KA6 | 3.18 | TSR3 | 2.7.37 |
| LAMP1 | 2.5 | PRRC2C | 2.8 | CCDC73 | 3.18 | INPP5E | 2.7.37 |
| CCDC80 | 2.5 | CD44 | 2.8 | SYCP2 | 3.18 | RBM42 | 2.7.37 |
| ENO1 | 2.5 | IQGAP1 | 2.8 | TECRL | 3.18 | RRP7A | 2.7.37 |
| DPYSL3 | 2.5 | TRAM1 | 2.8 | CADM2 | 3.18 | OR4S2 | 2.7.37 |
| SRSF11 | 2.5 | MTDH | 2.8 | LRRIQ3 | 3.18 | NINJ2 | 2.7.37 |
| IFITM2 | 2.5 | CCAR1 | 2.8 | GABRG1 | 3.18 | TWIST1 | 2.7.37 |
| PLBD2 | 2.5 | CAST | 2.8 | CDH7 | 3.18 | OR4C11 | 2.7.37 |
| RNASE1 | 2.5 | RBPJ | 2.8 | SPOCK3 | 3.18 | HRCT1 | 2.7.37 |
| LAMB2 | 2.5 | CANX | 2.8 | GLIPRIL2 | 3.18 | SYDE1 | 2.7.37 |
| TIMP3 | 2.5 | KMT2C | 2.8 | FSTL5 | 3.18 | IFNA10 | 2.7.37 |
| CD9 | 2.5 | TMED2 | 2.8 | LRRIQ | 3.18 | PODNL1 | 2.7.37 |
| BSG | 2.5 | PTPN12 | 2.8 | TMEFF2 | 3.18 | OR52N5 | 2.7.37 |
| SEC61A1 | 2.5 | EFCAB14 | 2.8 | SI | 3.18 | NICN1 | 2.7.37 |
| GLUL | 2.5 | ZEB2 | 2.8 | MGAT4D | 3.18 | ASIC5 | 2.7.37 |
| PRG4 | 2.5 | PICALM | 2.8 | EPHA5 | 3.18 | DYNC1H1 | 2.7.37 |
| PLP2 | 2.5 | HSP90B1 | 2.8 | STATH | 3.18 | HUWE1 | 2.7.37 |
| TNS1 | 2.5 | NPC2 | 2.8 | MYBPC1 | 3.18 | CHMP2A | 2.7.37 |
| COLGALT1 | 2.5 | SEC31A | 2.8 | ZNF711 | 3.18 | PRPF6 | 2.7.37 |
| ABHD2 | 2.5 | MBNL1 | 2.8 | EPYC | 3.18 | GOLGA3 | 2.7.37 |
| PRELP | 2.5 | EP300 | 2.8 | PI15 | 3.18 | ZNF362 | 2.7.37 |
| AXL | 2.5 | SEC63 | 2.8 | PRR27 | 3.18 | TCF25 | 2.7.37 |
| LRP1 | 2.5 | CALM2 | 2.8 | SNX16 | 3.18 | DNAJB1 | 2.7.37 |
| EMP1 | 2.5 | BPTF | 2.8 | C14orf39 | 3.18 | TALDO1 | 2.7.37 |
| CST3 | 2.5 | WNK1 | 2.8 | ANGPTL3 | 3.18 | SHC1 | 2.7.37 |
| SKI | 2.5 | CD164 | 2.8 | LUZP2 | 3.18 | FCGRT | 2.7.37 |
| FLII | 2.5 | UBXN4 | 2.8 | SLC6A15 | 3.18 | EIF4H | 2.7.37 |
| LTBP3 | 2.5 | TAOK1 | 2.8 | PLPPR5 | 3.18 | DEDD2 | 2.7.37 |
| SPARCL1 | 2.5 | SH3BGRL | 2.8 | MICU3 | 3.18 | COL4A1 | 2.7.37 |
| PLXNB2 | 2.5 | CLTC | 2.8 | GPR22 | 3.18 | GLIPR2 | 2.7.37 |
| ESYT2 | 2.5 | HP1BP3 | 2.8 | KLRC1 | 3.18 | TPRG1L | 2.7.37 |
| IGFBP4 | 2.5 | ATP6V1F | 2.8 | NEB | 3.18 | ADGRA2 | 2.7.37 |
| AEBP1 | 2.5 | ARGLU1 | 2.8 | OSTN | 3.18 | SUN2 | 2.7.37 |
| VSIG4 | 2.5 | SETX | 2.8 | CDH10 | 3.18 | OSR2 | 2.7.37 |
| MYH9 | 2.5 | ARHGDIB | 2.8 | KIF18A | 3.18 | CLUH | 2.7.37 |
| SPI1 | 2.5 | AHR | 2.8 | HCN1 | 3.18 | FPGS | 2.7.37 |
| ANGPTL2 | 2.5 | EIF3A | 2.8 | CSN3 | 3.18 | PPIH | 2.7.37 |
| PDGFRB | 2.5 | CREB3L2 | 2.8 | MYH7 | 3.18 | PKN1 | 2.7.37 |
| NDRG1 | 2.5 | EVI2B | 2.8 | RGS21 | 3.18 | ARF5 | 2.7.37 |
| PMEPA1 | 2.5 | SERINC1 | 2.8 | MYH1 | 3.18 | CRYAB | 2.7.37 |
| PLA2G2A | 2.5 | DEK | 2.8 | CA3 | 3.18 | IDUA | 2.7.37 |
| SELPLG | 2.5 | BRI3 | 2.8 | CFHR1 | 3.18 | CLIP3 | 2.7.37 |
| MAP4 | 2.5 | ASHIL | 2.8 | CKM | 3.18 | SAT2 | 2.7.37 |
| ASPN | 2.5 | LGALS3 | 2.8 | NECAB1 | 3.18 | NBPF14 | 2.7.37 |
| FPR3 | 2.5 | KLF6 | 2.8 | ACTA1 | 3.18 | FRAT2 | 2.7.37 |
| ATN1 | 2.5 | TMED10 | 2.8 | TAPBPL | 3.18 | RNASE11 | 2.7.37 |
| SCARA3 | 2.5 | SRGN | 2.8 | FANCB | 3.18 | AMELX | 2.7.37 |
| CHTF8 | 2.5 | KIF5B | 2.8 | MB | 3.18 | C3orf80 | 2.7.37 |
| FOLR2 | 2.5 | KCTD12 | 2.8 | NOL4 | 3.18 | SPATA25 | 2.7.37 |
| CRIP2 | 2.5 | EPB41L3 | 2.8 | MYL2 | 3.18 | SON | 2.8.41 |
| CTSL | 2.5 | SF1 | 2.8 | GJE1 | 3.18 | KMT2E | 2.8.41 |
| HSPG2 | 2.5 | SSR3 | 2.8 | F13B | 3.18 | PRRC2C | 2.8.41 |
| CYBRD1 | 2.5 | SUMO2 | 2.8 | MYOT | 3.18 | CD44 | 2.8.41 |
| TGFB1 | 2.5 | PTPRC | 2.8 | TNNI1 | 3.18 | IQGAP1 | 2.8.41 |
| GDI1 | 2.5 | HNRNPU | 2.8 | PLSCR5 | 3.18 | TRAM1 | 2.8.41 |
| ACKR3 | 2.5 | LCP1 | 2.8 | RAB3D | 3.18 | MTDH | 2.8.41 |
| AP1B1 | 2.5 | DNAJA1 | 2.8 | SLCO1B3 | 3.18 | CCAR1 | 2.8.41 |
| CLU | 2.5 | FAM120A | 2.8 | UGT2B7 | 3.18 | CAST | 2.8.41 |
| S100A8 | 2.5 | RAB10 | 2.8 | TRDN | 3.18 | RBPJ | 2.8.41 |
| NFE2L1 | 2.5 | HSPA5 | 2.8 | MYL1 | 3.18 | CANX | 2.8.41 |
| COL6A3 | 2.5 | PARP14 | 2.8 | KHDRBS2 | 3.18 | KMT2C | 2.8.41 |
| C3AR1 | 2.5 | LEPROT | 2.8 | NTS | 3.18 | TMED2 | 2.8.41 |
| CAMKID | 2.5 | RSU1 | 2.8 | UGT2B11 | 3.18 | PTPN12 | 2.8.41 |
| MS4A6A | 2.5 | DNAJC8 | 2.8 | ATP2A1 | 3.18 | EFCAB14 | 2.8.41 |
| FHL1 | 2.5 | HNRNPAO | 2.8 | TEX11 | 3.18 | ZEB2 | 2.8.41 |
| RAB35 | 2.5 | MTPN | 2.8 | ZDHHC24 | 3.18 | PICALM | 2.8.41 |
| CD14 | 2.5 | RABIA | 2.8 | TNNT1 | 3.18 | HSP90B1 | 2.8.41 |
| DUSP1 | 2.5 | CYBB | 2.8 | VPS37C | 3.18 | NPC2 | 2.8.41 |
| CLEC3B | 2.5 | TFG | 2.8 | PRX | 3.18 | SEC31A | 2.8.41 |
| MT-ND6 | 2.5 | STAT1 | 2.8 | MYEF2 | 3.18 | MBNL1 | 2.8.41 |
| AQP1 | 2.5 | SFPQ | 2.8 | XKR3 | 3.18 | EP300 | 2.8.41 |
| ANPEP | 2.5 | ARID5B | 2.8 | HECTD3 | 3.18 | SEC63 | 2.8.41 |
| C1S | 2.5 | EML4 | 2.8 | GKN1 | 3.18 | CALM2 | 2.8.41 |
| TBC1D14 | 2.5 | CIRBP | 2.8 | SPO11 | 3.18 | BPTF | 2.8.41 |
| MAPKAPK2 | 2.5 | FNDC3B | 2.8 | SGSM2 | 3.18 | WNK1 | 2.8.41 |
| APEX1 | 2.5 | CSNK1A1 | 2.8 | SLC37A2 | 3.18 | CD164 | 2.8.41 |
| IGFBP5 | 2.5 | CSTB | 2.8 | USP11 | 3.18 | UBXN4 | 2.8.41 |
| MGP | 2.5 | EVI2A | 2.8 | SH3BP1 | 3.18 | TAOK1 | 2.8.41 |
| DNM1 | 2.5 | MYO5A | 2.8 | HEXIM2 | 3.18 | SH3BGRL | 2.8.41 |
| ATF4 | 2.5 | ERH | 2.8 | ST14 | 3.18 | CLTC | 2.8.41 |
| FGFR1 | 2.5 | RAD23B | 2.8 | PLCD1 | 3.18 | HP1BP3 | 2.8.41 |
| SPTBN1 | 2.5 | POMP | 2.8 | AQP3 | 3.18 | ATP6V1F | 2.8.41 |
| VAT1 | 2.5 | UBE2K | 2.8 | XIRP1 | 3.18 | ARGLU1 | 2.8.41 |
| CDC37 | 2.5 | NCOA4 | 2.8 | TRMT61A | 3.18 | SETX | 2.8.41 |
| GPX3 | 2.5 | CORO1C | 2.8 | LIPI | 3.18 | AHR | 2.8.41 |
| MMP14 | 2.5 | MAPILC3B | 2.8 | IL5 | 3.18 | EIF3A | 2.8.41 |
| RRAS | 2.5 | PCNP | 2.8 | CAPS | 3.18 | CREB3L2 | 2.8.41 |
| NCOR2 | 2.5 | NOL7 | 2.8 | DGKQ | 3.18 | SERINC1 | 2.8.41 |
| ANTXR1 | 2.5 | CYP1B1 | 2.8 | ENO3 | 3.18 | DEK | 2.8.41 |
| COL5A1 | 2.5 | G3BP2 | 2.8 | SMPX | 3.18 | BRI3 | 2.8.41 |
| FTH1 | 2.5 | SAMD9L | 2.8 | OR10AG1 | 3.18 | ASHIL | 2.8.41 |
| DPT | 2.5 | FUCA1 | 2.8 | TRIM48 | 3.18 | LGALS3 | 2.8.41 |
| COL14A1 | 2.5 | EZR | 2.8 | HDAC11 | 3.18 | KLF6 | 2.8.41 |
| STAB1 | 2.5 | DBI | 2.8 | IFNA17 | 3.18 | TMED10 | 2.8.41 |
| COMP | 2.5 | FCGR3A | 2.8 | FRG2B | 3.18 | KIF5B | 2.8.41 |
| ZFP36 | 2.5 | RNF13 | 2.8 | AREG | 3.18 | KCTD12 | 2.8.41 |
| SZRD1 | 2.5 | QKI | 2.8 | TNNC1 | 3.18 | EPB41L3 | 2.8.41 |
| PLEC | 2.5 | APOL6 | 2.8 | SLCO4A1 | 3.18 | SF1 | 2.8.41 |
| BET1L | 2.5 | IRF2BP2 | 2.8 | PLEKHJ1 | 3.18 | SSR3 | 2.8.41 |
| ATP6AP1 | 2.5 | ZDHHC20 | 2.8 | KRTAP21-3 | 3.18 | SUMO2 | 2.8.41 |
| AKAP13 | 2.5 | DNAJC1 | 2.8 | POLRMT | 3.18 | HNRNPU | 2.8.41 |
| TNFRSF1A | 2.5 | STARD7 | 2.8 | DEFB112 | 3.18 | DNAJA1 | 2.8.41 |
| PFKP | 2.5 | GALNT1 | 2.8 | TMSB15B | 3.18 | FAM120A | 2.8.41 |
| EMB | 2.5 | WIPF1 | 2.8 | TNNT3 | 3.18 | RAB10 | 2.8.41 |
| CD58 | 2.5 | C5orf15 | 2.8 | NDST3 | 3.18 | HSPA5 | 2.8.41 |
| LHFPL2 | 2.5 | ITGA6 | 2.8 | TREML1 | 3.18 | LEPROT | 2.8.41 |
| EHD2 | 2.5 | APOL1 | 2.8 | SPINK1 | 3.18 | RSU1 | 2.8.41 |
| LAMC1 | 2.5 | PJA2 | 2.8 | FOSL1 | 3.18 | DNAJC8 | 2.8.41 |
| MYO1C | 2.5 | OGT | 2.8 | ALG12 | 3.18 | HNRNPAO | 2.8.41 |
| CD151 | 2.5 | PLEK | 2.8 | FBXO2 | 3.18 | MTPN | 2.8.41 |
| PFKL | 2.5 | IL1R1 | 2.8 | TEX12 | 3.18 | RABIA | 2.8.41 |
| ARHGDIA | 2.5 | CD302 | 2.8 | HLA-DRB5 | 3.18 | CYBB | 2.8.41 |
| QRICH1 | 2.5 | ARHGAP18 | 2.8 | MYL3 | 3.18 | TFG | 2.8.41 |
| PNRC1 | 2.5 | CD53 | 2.8 | LRRC29 | 3.18 | SFPQ | 2.8.41 |
| MNDA | 2.5 | METTL7A | 2.8 | C1QL4 | 3.18 | ARID5B | 2.8.41 |
| MT2A | 2.5 | LYZ | 2.8 | PRR36 | 3.18 | CIRBP | 2.8.41 |
| IDH3G | 2.5 | BASP1 | 2.8 | EEF1A2 | 3.18 | FNDC3B | 2.8.41 |
| RAB1B | 2.5 | CTCF | 2.8 | OGFOD2 | 3.18 | CSNK1A1 | 2.8.41 |
| PITPNA | 2.5 | SEMA3C | 2.8 | REEP6 | 3.18 | CSTB | 2.8.41 |
| MYD88 | 2.5 | CLK1 | 2.8 | EEF2KMT | 3.18 | EVI2A | 2.8.41 |
| CSPG4 | 2.5 | CD47 | 2.8 | CFHR3 | 3.18 | MYO5A | 2.8.41 |
| HK1 | 2.5 | GBP1 | 2.8 | NPIPB11 | 3.18 | ERH | 2.8.41 |
| CD34 | 2.5 | GBP2 | 2.8 | POTEB3 | 3.18 | RAD23B | 2.8.41 |
| CTSZ | 2.5 | CFH | 2.8 | CARD9 | 3.18 | POMP | 2.8.41 |
| TRIM8 | 2.5 | CSF2RB | 2.8 | VCX3A | 3.18 | UBE2K | 2.8.41 |
| MARCO | 2.5 | PSPC1 | 2.8 | FUOM | 3.18 | NCOA4 | 2.8.41 |
| DPP7 | 2.5 | RPS6KA3 | 2.8 | LRRC70 | 3.18 | CORO1C | 2.8.41 |
| S100A9 | 2.5 | AHCYL1 | 2.8 | SAC3D1 | 3.18 | MAPILC3B | 2.8.41 |
| CAPG | 2.5 | SLC25A3 | 2.8 | CYLC2 | 3.18 | PCNP | 2.8.41 |
| SNED1 | 2.5 | FOXN3 | 2.8 | CST1 | 3.18 | NOL7 | 2.8.41 |
| TSC22D3 | 2.5 | GNS | 2.8 | NBPF4 | 3.18 | CYP1B1 | 2.8.41 |
| SCARA5 | 2.5 | CCL2 | 2.8 | EPHA7 | 3.18 | G3BP2 | 2.8.41 |
| MRPL40 | 2.5 | SMAP2 | 2.8 | TCAP | 3.18 | FUCA1 | 2.8.41 |
| CERCAM | 2.5 | REEP5 | 2.8 | TNNC2 | 3.18 | EZR | 2.8.41 |
| PDPN | 2.5 | GDI2 | 2.8 | OR5H15 | 3.18 | DBI | 2.8.41 |
| MARVELD1 | 2.5 | LITAF | 2.8 | CTNNBIP1 | 3.18 | FCGR3A | 2.8.41 |
| GOLM1 | 2.5 | SNX6 | 2.8 | GGT1 | 3.18 | RNF13 | 2.8.41 |
| CIC | 2.5 | CHCHD2 | 2.8 | KRTAP20-4 | 3.18 | QKI | 2.8.41 |
| KANK2 | 2.5 | TRIM14 | 2.8 | TSSK6 | 3.18 | IRF2BP2 | 2.8.41 |
| CCR1 | 2.5 | DOK3 | 2.8 | E2F1 | 3.18 | ZDHHC20 | 2.8.41 |
| PIP5K1C | 2.5 | SOD2 | 2.8 | COX7B2 | 3.18 | DNAJC1 | 2.8.41 |
| CCDC71 | 2.5 | PIM3 | 2.8 | SLITRK5 | 3.18 | GALNT1 | 2.8.41 |
| CMKLR1 | 2.5 | PGRMC1 | 2.8 | HFM1 | 3.18 | C5orf15 | 2.8.41 |
| THBS3 | 2.5 | CTDNEP1 | 2.8 | TMPRSS11A | 3.18 | ITGA6 | 2.8.41 |
| GSN | 2.5 | ZBTB7A | 2.8 | CYLC1 | 3.18 | APOL1 | 2.8.41 |
| MS4A7 | 2.5 | IRF2 | 2.8 | PLCZ1 | 3.18 | PJA2 | 2.8.41 |
| TLN1 | 2.5 | VAMP2 | 2.8 | CFHR4 | 3.18 | OGT | 2.8.41 |
| TGFBR3 | 2.5 | IFI44L | 2.8 | ZSWIM2 | 3.18 | PLEK | 2.8.41 |
| HSD3B7 | 2.5 | PRPF18 | 2.8 | MYH2 | 3.18 | IL1R1 | 2.8.41 |
| GPR137B | 2.5 | PSMG2 | 2.8 | GLIPRIL1 | 3.18 | CD302 | 2.8.41 |
| ARPC4 | 2.5 | SNX10 | 2.8 | CDK1 | 3.18 | ARHGAP18 | 2.8.41 |
| SORBS3 | 2.5 | PLEKHO2 | 2.8 | GRB14 | 3.18 | METTL7A | 2.8.41 |
| HAVCR2 | 2.5 | CCND2 | 2.8 | ZCWPW2 | 3.18 | BASP1 | 2.8.41 |
| NNMT | 2.5 | CLTA | 2.8 | OR5M3 | 3.18 | CTCF | 2.8.41 |
| PER2 | 2.5 | PRELID3B | 2.8 | LRRC72 | 3.18 | SEMA3C | 2.8.41 |
| SLC2A4RG | 2.5 | CLEC2B | 2.8 | DDA1 | 3.18 | CLK1 | 2.8.41 |
| HIVEP2 | 2.5 | ABCA1 | 2.8 | FABP2 | 3.18 | CD47 | 2.8.41 |
| ITPR3 | 2.5 | GP2 | 2.8 | MAEL | 3.18 | CFH | 2.8.41 |
| CAPN2 | 2.5 | DMRTC1B | 2.8 | DDX51 | 3.18 | PSPC1 | 2.8.41 |
| FMOD | 2.5 | LAP3 | 2.8 | MYOZ1 | 3.18 | RPS6KA3 | 2.8.41 |
| PTGES | 2.5 | ITGB8 | 2.8 | MYBPC2 | 3.18 | AHCYL1 | 2.8.41 |
| LRRC15 | 2.5 | TOP1 | 2.8 | PYGM | 3.18 | SLC25A3 | 2.8.41 |
| ADAMTSL4 | 2.5 | RASSF2 | 2.8 | RIPK3 | 3.18 | FOXN3 | 2.8.41 |
| COMT | 2.5 | HIGD2A | 2.8 | CCDC137 | 3.18 | GNS | 2.8.41 |
| CDK11B | 2.5 | STRAP | 2.8 | STARD10 | 3.18 | CCL2 | 2.8.41 |
| SFXN3 | 2.5 | MRPS34 | 2.8 | DEFB113 | 3.18 | REEP5 | 2.8.41 |
| FNDC1 | 2.5 | LALBA | 2.8 | BORCS6 | 3.18 | GDI2 | 2.8.41 |
| GAS7 | 2.5 | BHLHE41 | 2.8 | PMCH | 3.18 | SNX6 | 2.8.41 |
| THY1 | 2.5 | C4orf3 | 2.8 | CCDC179 | 3.18 | CHCHD2 | 2.8.41 |
| NR2F2 | 2.5 | TNFAIP6 | 2.8 | ZNF404 | 3.18 | TRIM14 | 2.8.41 |
| Clorf162 | 2.5 | SF3B6 | 2.8 | SLC19A1 | 3.18 | PIM3 | 2.8.41 |
| KLF4 | 2.5 | CD163 | 2.8 | C1QTNF9B | 3.18 | PGRMC1 | 2.8.41 |
| TPPP | 2.5 | TBCB | 2.8 | GCSH | 3.18 | CTDNEP1 | 2.8.41 |
| TUFM | 2.5 | ROCK1 | 2.8 | TNNI2 | 3.18 | ZBTB7A | 2.8.41 |
| KLF9 | 2.5 | UBL4A | 2.8 | FITM1 | 3.18 | IRF2 | 2.8.41 |
| TRAPPC1 | 2.5 | SLC34A2 | 2.8 | FOXJ1 | 3.18 | VAMP2 | 2.8.41 |
| MXRA8 | 2.5 | CDKN2D | 2.8 | USP17L1 | 3.18 | PRPF18 | 2.8.41 |
| NBL1 | 2.5 | BAZIA | 2.8 | DNAAF3 | 3.18 | PSMG2 | 2.8.41 |
| PCOLCE2 | 2.5 | FCGR1A | 2.8 | NUTM2B | 3.18 | PLEKHO2 | 2.8.41 |
| SDF4 | 2.5 | TRIB1 | 2.8 | KRTAP12-3 | 3.18 | CCND2 | 2.8.41 |
| IGF2 | 2.5 | OR4K1 | 2.8 | LBX2 | 3.18 | CLTA | 2.8.41 |
| RBM3 | 2.5 | PARP4 | 2.8 | MT-ATP6 | 2.5.19 | PRELID3B | 2.8.41 |
| SRM | 2.5 | RPS27L | 2.8 | MT-ND2 | 2.5.19 | ABCA1 | 2.8.41 |
| STAT3 | 2.5 | IFI44 | 2.8 | MT-ND1 | 2.5.19 | GP2 | 2.8.41 |
| PHPT1 | 2.5 | SRSF1 | 2.8 | MT-CO1 | 2.5.19 | DMRTC1B | 2.8.41 |
| GFPT2 | 2.5 | HIGDIA | 2.8 | MT-CYB | 2.5.19 | ITGB8 | 2.8.41 |
| PKD1 | 2.5 | TMEM59 | 2.8 | UBC | 2.5.19 | TOP1 | 2.8.41 |
| TTYH3 | 2.5 | DEFB1 | 2.8 | ACTB | 2.5.19 | HIGD2A | 2.8.41 |
| ARL2 | 2.5 | PDCDILG2 | 2.8 | ZFP36L1 | 2.5.19 | STRAP | 2.8.41 |
| GJA1 | 2.5 | FILIP1L | 2.8 | ACTG1 | 2.5.19 | MRPS34 | 2.8.41 |
| SOD1 | 2.5 | PLBD1 | 2.8 | PTMA | 2.5.19 | LALBA | 2.8.41 |
| PLXND1 | 2.5 | CCL13 | 2.8 | COL3A1 | 2.5.19 | BHLHE41 | 2.8.41 |
| POSTN | 2.5 | PYCARD | 2.8 | RPL8 | 2.5.19 | C4orf3 | 2.8.41 |
| CALM3 | 2.5 | TAS2R60 | 2.8 | ANP32B | 2.5.19 | TNFAIP6 | 2.8.41 |
| SHARPIN | 2.5 | CPA1 | 2.8 | COL6A1 | 2.5.19 | SF3B6 | 2.8.41 |
| TMEM119 | 2.5 | EIF3J | 2.8 | MT-ND5 | 2.5.19 | CD163 | 2.8.41 |
| GNG12 | 2.5 | RAB6A | 2.8 | MT-ND4L | 2.5.19 | TBCB | 2.8.41 |
| CRLF1 | 2.5 | CDKN1B | 2.8 | PMP22 | 2.5.19 | ROCK1 | 2.8.41 |
| ZNF503 | 2.5 | ACTR2 | 2.8 | ARF1 | 2.5.19 | UBL4A | 2.8.41 |
| ZNF768 | 2.5 | SOCS3 | 2.8 | VIM | 2.5.19 | CDKN2D | 2.8.41 |
| BLVRB | 2.5 | STEAP1 | 2.8 | TXNIP | 2.5.19 | FCGRIA | 2.8.41 |
| MGAT4B | 2.5 | DRAP1 | 2.8 | CD63 | 2.5.19 | OR4K1 | 2.8.41 |
| PYGB | 2.5 | HCST | 2.8 | PSAP | 2.5.19 | PARP4 | 2.8.41 |
| ATL3 | 2.5 | STEAP4 | 2.8 | SERPING1 | 2.5.19 | RPS27L | 2.8.41 |
| LYVE1 | 2.5 | LEP | 2.8 | TMSB10 | 2.5.19 | SRSF1 | 2.8.41 |
| ACTN4 | 2.5 | TNFRSF1B | 2.8 | TIMP2 | 2.5.19 | HIGDIA | 2.8.41 |
| SLC25A5 | 2.5 | EXTL1 | 2.8 | DDX17 | 2.5.19 | TMEM59 | 2.8.41 |
| IGFBP6 | 2.5 | HOXC11 | 2.8 | MT-CO2 | 2.5.19 | DEFB1 | 2.8.41 |
| GOLGA7B | 2.5 | TPSAB1 | 2.8 | EIF1 | 2.5.19 | PDCDILG2 | 2.8.41 |
| TTC7A | 2.5 | CIB3 | 2.8 | TMSB4X | 2.5.19 | FILIP1L | 2.8.41 |
| LRRN4CL | 2.5 | OR10H5 | 2.8 | MSN | 2.5.19 | PLBD1 | 2.8.41 |
| CCNI | 2.5 | DUSP6 | 2.8 | BGN | 2.5.19 | CCL13 | 2.8.41 |
| GNA11 | 2.5 | MSR1 | 2.8 | LUC7L3 | 2.5.19 | PYCARD | 2.8.41 |
| EGR1 | 2.5 | ARL4C | 2.8 | FTL | 2.5.19 | TAS2R60 | 2.8.41 |
| CHID1 | 2.5 | CST7 | 2.8 | FLNA | 2.5.19 | CPA1 | 2.8.41 |
| THBS2 | 2.5 | SLC43A3 | 2.8 | EEF2 | 2.5.19 | EIF3J | 2.8.41 |
| NCF2 | 2.5 | OR4K5 | 2.8 | ISLR | 2.5.19 | RAB6A | 2.8.41 |
| TSPAN15 | 2.5 | DGCR6L | 2.8 | CYB5R3 | 2.5.19 | ACTR2 | 2.8.41 |
| NFIA | 2.5 | OR52M1 | 2.8 | ZFP36L2 | 2.5.19 | SOCS3 | 2.8.41 |
| NOTCH3 | 2.5 | OR4N2 | 2.8 | OAZ1 | 2.5.19 | STEAP1 | 2.8.41 |
| MAPKAPK3 | 2.5 | BCAT1 | 2.8 | AHNAK | 2.5.19 | DRAP1 | 2.8.41 |
| POLR2J | 2.5 | MS4A15 | 2.8 | CD81 | 2.5.19 | STEAP4 | 2.8.41 |
| SIGLEC1 | 2.5 | CLEC4M | 2.8 | GNAI2 | 2.5.19 | LEP | 2.8.41 |
| PTPRS | 2.5 | IFIH1 | 2.8 | PCBP1 | 2.5.19 | HOXC11 | 2.8.41 |
| KDELR2 | 2.5 | IL25 | 2.8 | COL6A2 | 2.5.19 | TPSAB1 | 2.8.41 |
| SLC12A4 | 2.5 | OR2M2 | 2.8 | NFIX | 2.5.19 | CIB3 | 2.8.41 |
| ABL1 | 2.5 | LYZL4 | 2.8 | LGALS1 | 2.5.19 | OR10H5 | 2.8.41 |
| GNB2 | 2.5 | NYX | 2.8 | COL1A2 | 2.5.19 | DUSP6 | 2.8.41 |
| FABP3 | 2.5 | PRKACG | 2.8 | HTRA1 | 2.5.19 | MSR1 | 2.8.41 |
| AGPAT2 | 2.5 | UBL4B | 2.8 | SPARC | 2.5.19 | SLC43A3 | 2.8.41 |
| RGCC | 2.5 | FOXF2 | 2.8 | FN1 | 2.5.19 | OR4K5 | 2.8.41 |
| ADCY7 | 2.5 | NR113 | 2.8 | JUND | 2.5.19 | DGCR6L | 2.8.41 |
| EIF3I | 2.5 | GPRC5D | 2.8 | IGFBP7 | 2.5.19 | OR52M1 | 2.8.41 |
| ADAMTS2 | 2.5 | SMIM24 | 2.8 | PPDPF | 2.5.19 | OR4N2 | 2.8.41 |
| LTBP4 | 2.5 | HAND1 | 2.8 | COL1A1 | 2.5.19 | BCAT1 | 2.8.41 |
| PLXNA3 | 2.5 | SOX18 | 2.8 | LAMP1 | 2.5.19 | MS4A15 | 2.8.41 |
| GALNT15 | 2.5 | RNF168 | 2.8 | SRSF11 | 2.5.19 | CLEC4M | 2.8.41 |
| DCHS1 | 2.5 | ASIC4 | 2.8 | IFITM2 | 2.5.19 | IL25 | 2.8.41 |
| CXCL14 | 2.5 | SEMA5B | 2.8 | TIMP3 | 2.5.19 | OR2M2 | 2.8.41 |
| SLC39A13 | 2.5 | CXCL10 | 2.8 | CD9 | 2.5.19 | NYX | 2.8.41 |
| TMEM47 | 2.5 | C15orf48 | 2.8 | BSG | 2.5.19 | PRKACG | 2.8.41 |
| DKK3 | 2.5 | SGCA | 2.8 | PRG4 | 2.5.19 | UBL4B | 2.8.41 |
| CDH23 | 2.5 | OR2V1 | 2.8 | PLP2 | 2.5.19 | FOXF2 | 2.8.41 |
| IGF1 | 2.5 | GZMA | 2.8 | LRP1 | 2.5.19 | NR113 | 2.8.41 |
| SFRP2 | 2.5 | PRKARIB | 2.8 | FLII | 2.5.19 | GPRC5D | 2.8.41 |
| CHI3L2 | 2.5 | GLP1R | 2.8 | SPARCL1 | 2.5.19 | SMIM24 | 2.8.41 |
| CRTAC1 | 2.5 | SPP1 | 2.8 | PLXNB2 | 2.5.19 | HAND1 | 2.8.41 |
| CILP | 2.5 | SPRR2G | 2.8 | MYH9 | 2.5.19 | RNF168 | 2.8.41 |
| SHANK3 | 2.5 | SERPINA11 | 2.8 | CHTF8 | 2.5.19 | ASIC4 | 2.8.41 |
| LOXL1 | 2.5 | PROKR2 | 2.8 | CRIP2 | 2.5.19 | SGCA | 2.8.41 |
| GPR153 | 2.5 | ARCN1 | 2.8 | TGFB1 | 2.5.19 | OR2V1 | 2.8.41 |
| SFRP4 | 2.5 | CXorf66 | 2.8 | CLU | 2.5.19 | PRKARIB | 2.8.41 |
| FKBP8 | 2.5 | COX5B | 2.8 | S100A8 | 2.5.19 | GLPIR | 2.8.41 |
| PER1 | 2.5 | GBP5 | 2.8 | COL6A3 | 2.5.19 | SPP1 | 2.8.41 |
| CORO1B | 2.5 | KCNQ4 | 2.8 | FHL1 | 2.5.19 | SPRR2G | 2.8.41 |
| COL18A1 | 2.5 | TFDP3 | 2.8 | RAB35 | 2.5.19 | SERPINA11 | 2.8.41 |
| FAM20C | 2.5 | DNAJB13 | 2.8 | DUSP1 | 2.5.19 | ARCN1 | 2.8.41 |
| HMCN2 | 2.5 | UCKL1 | 2.8 | MT-ND6 | 2.5.19 | CXorf66 | 2.8.41 |
| CNN3 | 2.5 | IGSF6 | 2.8 | IGFBP5 | 2.5.19 | COX5B | 2.8.41 |
| HCFC1R1 | 2.5 | GIMAP7 | 2.8 | ATF4 | 2.5.19 | TFDP3 | 2.8.41 |
| HADHA | 2.5 | HDDC3 | 2.8 | SPTBN1 | 2.5.19 | DNAJB13 | 2.8.41 |
| CDIPT | 2.5 | DNAI2 | 2.8 | VAT1 | 2.5.19 | UCKL1 | 2.8.41 |
| RNASE6 | 2.5 | MOS | 2.8 | CDC37 | 2.5.19 | HDDC3 | 2.8.41 |
| MRFAP1 | 2.5 | TEKT5 | 2.8 | MMP14 | 2.5.19 | OR7D4 | 2.8.41 |
| NDUFA4L2 | 2.5 | CSDC2 | 2.8 | RRAS | 2.5.19 | LY86 | 2.8.41 |
| UBACI | 2.5 | OR7D4 | 2.8 | NCOR2 | 2.5.19 | CLCNKA | 2.8.41 |
| NTN1 | 2.5 | DTX3L | 2.8 | COL5A1 | 2.5.19 | C2orf72 | 2.8.41 |
| MRPS21 | 2.5 | LY86 | 2.8 | FTH1 | 2.5.19 | C16orf54 | 2.8.41 |
| MAMDC2 | 2.5 | ANKK1 | 2.8 | STAB1 | 2.5.19 | ACBD3 | 2.8.41 |
| PPP1R15A | 2.5 | CLCNKA | 2.8 | ZFP36 | 2.5.19 | TFF1 | 2.8.41 |
| IER5 | 2.5 | C2orf72 | 2.8 | PLEC | 2.5.19 | Clorf226 | 2.8.41 |
| MED16 | 2.5 | C16orf54 | 2.8 | EHD2 | 2.5.19 | KCNE5 | 2.8.41 |
| SLC31A1 | 2.5 | ACBD3 | 2.8 | LAMC1 | 2.5.19 | DDX1 | 2.8.41 |
| TCIRG1 | 2.5 | OR10G9 | 2.8 | CD151 | 2.5.19 | KRTAP10-9 | 2.8.41 |
| ITGB4 | 2.5 | TFF1 | 2.8 | ARHGDIA | 2.5.19 | NUDT16L1 | 2.8.41 |
| VPS28 | 2.5 | Clorf226 | 2.8 | PNRC1 | 2.5.19 | HIPK4 | 2.8.41 |
| ICMT | 2.5 | NAMPT | 2.8 | MNDA | 2.5.19 | PENK | 2.8.41 |
| WDR45B | 2.5 | KCNE5 | 2.8 | MT2A | 2.5.19 | CLMP | 2.8.41 |
| CHI3L1 | 2.5 | DDX1 | 2.8 | IDH3G | 2.5.19 | HLA-B | 2.8.41 |
| RAP2B | 2.5 | KRT19 | 2.8 | CD34 | 2.5.19 | ZNF219 | 2.8.41 |
| HNRNPH2 | 2.5 | KRTAP10-9 | 2.8 | TRIM8 | 2.5.19 | IRF2BP1 | 2.8.41 |
| CEP170B | 2.5 | FAM131C | 2.8 | DPP7 | 2.5.19 | IL17B | 2.8.41 |
| ARRDC1 | 2.5 | NUDT16L1 | 2.8 | S100A9 | 2.5.19 | GPR182 | 2.8.41 |
| GADD45B | 2.5 | CCL26 | 2.8 | SNED1 | 2.5.19 | DOK7 | 2.8.41 |
| GPER1 | 2.5 | HIPK4 | 2.8 | TSC22D3 | 2.5.19 | IL36A | 2.8.41 |
| EMILIN2 | 2.5 | PENK | 2.8 | MARVELD1 | 2.5.19 | TEX101 | 2.8.41 |
| SNCG | 2.5 | EPHX3 | 2.8 | GSN | 2.5.19 | NHLH1 | 2.8.41 |
| ADM | 2.5 | CLMP | 2.8 | TLN1 | 2.5.19 | GOLGA8K | 2.8.41 |
| ADRM1 | 2.5 | HLA-B | 2.8 | SLC2A4RG | 2.5.19 | HTR1E | 2.8.41 |
| INTS3 | 2.5 | ZNF219 | 2.8 | CDK11B | 2.5.19 | OR5AU1 | 2.8.41 |
| HEXB | 2.5 | IRF2BP1 | 2.8 | FNDC1 | 2.5.19 | GSG1L2 | 2.8.41 |
| B4GALT2 | 2.5 | IL17B | 2.8 | NBL1 | 2.5.19 | IL3 | 2.8.41 |
| ALOX5AP | 2.5 | GPR182 | 2.8 | SDF4 | 2.5.19 | FAM86B1 | 2.8.41 |
| VPS9D1 | 2.5 | DOK7 | 2.8 | RBM3 | 2.5.19 | TPSD1 | 2.8.41 |
| COL8A2 | 2.5 | IL36A | 2.8 | SRM | 2.5.19 | SCX | 2.8.41 |
| AHNAK2 | 2.5 | WSCD2 | 2.8 | PHPT1 | 2.5.19 | MT1F | 2.8.41 |
| MBOAT4 | 2.5 | ACAN | 2.8 | SOD1 | 2.5.19 | CLDN6 | 2.8.41 |
| GUCD1 | 2.5 | TEX101 | 2.8 | GNG12 | 2.5.19 | KLHL30 | 2.8.41 |
| IL13RA1 | 2.5 | NHLH1 | 2.8 | ATL3 | 2.5.19 | LYG1 | 2.8.41 |
| AQP9 | 2.5 | HMX1 | 2.8 | ACTN4 | 2.5.19 | TRIM34 | 2.8.41 |
| MT-ND3 | 2.5 | GOLGA8K | 2.8 | IGFBP6 | 2.5.19 | CHAD | 2.8.41 |
| KATNB1 | 2.5 | HTR1E | 2.8 | GOLGA7B | 2.5.19 | OR4F5 | 2.8.41 |
| LSS | 2.5 | OR5AU1 | 2.8 | LRRN4CL | 2.5.19 | PDXDC1 | 2.8.41 |
| MAPIA | 2.5 | GSG1L2 | 2.8 | CCNI | 2.5.19 | TMSB15A | 2.8.41 |
| PGK1 | 2.5 | IL3 | 2.8 | EGR1 | 2.5.19 | PCBP4 | 2.8.41 |
| MFAP5 | 2.5 | FAM86B1 | 2.8 | THBS2 | 2.5.19 | C20orf27 | 2.8.41 |
| FOS | 2.5 | TPSD1 | 2.8 | NCF2 | 2.5.19 | OR3A3 | 2.8.41 |
| ABHD14B | 2.5 | SCX | 2.8 | POLR2J | 2.5.19 | VCAM1 | 2.8.41 |
| MAFK | 2.5 | MT1F | 2.8 | AGPAT2 | 2.5.19 | RNASE2 | 2.8.41 |
| OGFR | 2.5 | CLDN6 | 2.8 | ADAMTS2 | 2.5.19 | SRGAP2B | 2.8.41 |
| LAMA5 | 2.5 | KLHL30 | 2.8 | LTBP4 | 2.5.19 | FAM72C | 2.8.41 |
| NDUFV1 | 2.5 | LYG1 | 2.8 | TMEM47 | 2.5.19 | FXYD7 | 2.8.41 |
| ADIPOR1 | 2.5 | TRIM34 | 2.8 | CRTAC1 | 2.5.19 | PMVK | 2.8.41 |
| COL15A1 | 2.5 | CHAD | 2.8 | LOXL1 | 2.5.19 | IFNB1 | 2.8.41 |
| OR1J4 | 2.5 | RAB24 | 2.8 | GPR153 | 2.5.19 | DMRTC1 | 2.8.41 |
| MYO15B | 2.5 | OR4F5 | 2.8 | FKBP8 | 2.5.19 | TMEM249 | 2.8.41 |
| ST6GALNAC6 | 2.5 | PDXDC1 | 2.8 | FAM20C | 2.5.19 | TC2N | 2.8.41 |
| DYNLT1 | 2.5 | TMSB15A | 2.8 | HCFC1R1 | 2.5.19 | SPNS3 | 2.8.41 |
| REXO1 | 2.5 | PCDHGB1 | 2.8 | HADHA | 2.5.19 | SYNRG | 2.8.41 |
| SGSH | 2.5 | PCBP4 | 2.8 | MRPS21 | 2.5.19 | OR4K2 | 2.8.41 |
| PRRX2 | 2.5 | SLC35G4 | 2.8 | IER5 | 2.5.19 | KIF26A | 2.8.41 |
| PTTG2 | 2.5 | C20orf27 | 2.8 | VPS28 | 2.5.19 | FAAP24 | 2.8.41 |
| UAP1 | 2.5 | OR3A3 | 2.8 | ICMT | 2.5.19 | ZFP92 | 2.8.41 |
| ABCD1 | 2.5 | VCAM1 | 2.8 | HNRNPH2 | 2.5.19 | CBR3 | 2.8.41 |
| SERPINE1 | 2.5 | RHOD | 2.8 | GADD45B | 2.5.19 | TRPV1 | 2.8.41 |
| CIQA | 2.5 | RNASE2 | 2.8 | ADM | 2.5.19 | SP1 | 2.8.41 |
| NBPF10 | 2.5 | SRGAP2B | 2.8 | B4GALT2 | 2.5.19 | AFF4 | 2.8.41 |
| FLNC | 2.5 | FAM72C | 2.8 | AQP9 | 2.5.19 | IPO7 | 2.8.41 |
| SERPINF1 | 2.5 | PGLYRP1 | 2.8 | MT-ND3 | 2.5.19 | MEF2A | 2.8.41 |
| NKG7 | 2.5 | IGLL1 | 2.8 | FOS | 2.5.19 | PPP3CA | 2.8.41 |
| EGFL7 | 2.5 | FXYD7 | 2.8 | ADIPOR1 | 2.5.19 | ADD3 | 2.8.41 |
| ANGPTL7 | 2.5 | RRAD | 2.8 | COL15A1 | 2.5.19 | SPEN | 2.8.41 |
| RAB40C | 2.5 | CSTA | 2.8 | ST6GALNAC6 | 2.5.19 | MAF | 2.8.41 |
| CA12 | 2.5 | VCX3B | 2.8 | REXO1 | 2.5.19 | SLAIN2 | 2.8.41 |
| HOXD10 | 2.5 | PMVK | 2.8 | PRRX2 | 2.5.19 | SEC24B | 2.8.41 |
| FAM205A | 2.5 | TMEM88 | 2.8 | NKG7 | 2.5.19 | SAP18 | 2.8.41 |
| ERF | 2.5 | IFNB1 | 2.8 | ERF | 2.5.19 | C11orf58 | 2.8.41 |
| FOSB | 2.5 | ADM5 | 2.8 | FOSB | 2.5.19 | RAB2A | 2.8.41 |
| NKX2-1 | 2.5 | DMRTC1 | 2.8 | ZDHHC12 | 2.5.19 | VAMP3 | 2.8.41 |
| ZDHHC12 | 2.5 | TMEM249 | 2.8 | SMARCA4 | 2.5.19 | CTNNA1 | 2.8.41 |
| SMARCA4 | 2.5 | TC2N | 2.8 | CDC34 | 2.5.19 | NCKAPIL | 2.8.41 |
| CDC34 | 2.5 | SPNS3 | 2.8 | PRDX2 | 2.5.19 | TMEM248 | 2.8.41 |
| TUBGCP6 | 2.5 | SYNRG | 2.8 | ZBTB45 | 2.5.19 | SLFN5 | 2.8.41 |
| PRDX2 | 2.5 | GZMM | 2.8 | CRABP2 | 2.5.19 | ATP2B1 | 2.8.41 |
| CDT1 | 2.5 | ZSCAN10 | 2.8 | ZNF296 | 2.5.19 | TMPRSS2 | 2.8.41 |
| TSKU | 2.5 | SLC25A2 | 2.8 | MEGF6 | 2.5.19 | MBD2 | 2.8.41 |
| SLC52A2 | 2.5 | OR4K2 | 2.8 | URAD | 2.5.19 | ZC3H6 | 2.8.41 |
| ZBTB45 | 2.5 | KIF26A | 2.8 | NDUFB8 | 2.5.19 | MAX | 2.8.41 |
| FZD2 | 2.5 | FAAP24 | 2.8 | PPP1R16A | 2.5.19 | MBNL2 | 2.8.41 |
| TPRA1 | 2.5 | ZFP92 | 2.8 | ALPL | 2.5.19 | MRC1 | 2.8.41 |
| CRABP2 | 2.5 | LCP2 | 2.8 | ENTPD2 | 2.5.19 | DDX3X | 2.8.41 |
| LRFN4 | 2.5 | CBR3 | 2.8 | MT-CO3 | 2.5.19 | THOC7 | 2.8.41 |
| KLF13 | 2.5 | TRPV1 | 2.8 | LZTS2 | 2.5.19 | NFKBIZ | 2.8.41 |
| ZBTB17 | 2.5 | SP1 | 2.8 | LMANIL | 2.5.19 | PTGES3 | 2.8.41 |
| TINAGL1 | 2.5 | AFF4 | 2.8 | ADAM33 | 2.5.19 | MFSD14A | 2.8.41 |
| TNS2 | 2.5 | IPO7 | 2.8 | CCDC105 | 2.5.19 | DIAPH1 | 2.8.41 |
| HAGHL | 2.5 | MEF2A | 2.8 | NNAT | 2.5.19 | ARID5A | 2.8.41 |
| CPXM1 | 2.5 | PPP3CA | 2.8 | OR2T33 | 2.5.19 | HMGN3 | 2.8.41 |
| TSPAN4 | 2.5 | ADD3 | 2.8 | MT-ND4 | 2.5.19 | POLE3 | 2.8.41 |
| FZD10 | 2.5 | SPEN | 2.8 | NCL | 2.5.19 | LPXN | 2.8.41 |
| ZNF296 | 2.5 | MAF | 2.8 | GAPDH | 2.5.19 | RNF11 | 2.8.41 |
| RAB3IL1 | 2.5 | SLAIN2 | 2.8 | SH3BGRL3 | 2.5.19 | AKAP3 | 2.8.41 |
| GAS2L1 | 2.5 | SEC24B | 2.8 | SLC25A6 | 2.5.19 | AP2S1 | 2.8.41 |
| CLDN5 | 2.5 | SAP18 | 2.8 | CALHM2 | 2.5.19 | CCT4 | 2.8.41 |
| LRRC52 | 2.5 | C11orf58 | 2.8 | GPX1 | 2.5.19 | MYDGF | 2.8.41 |
| MEGF6 | 2.5 | RAB2A | 2.8 | DDX5 | 2.5.19 | KLK6 | 2.8.41 |
| HLA-DRB1 | 2.5 | VAMP3 | 2.8 | ABI3BP | 2.5.19 | KCNK9 | 2.8.41 |
| CALML4 | 2.5 | CTNNA1 | 2.8 | MCL1 | 2.5.19 | FER1L5 | 2.8.41 |
| URAD | 2.5 | NCKAPIL | 2.8 | MRC2 | 2.5.19 | KNG1 | 2.8.41 |
| TFEB | 2.5 | TMEM248 | 2.8 | TSPO | 2.5.19 | SCN5A | 2.8.41 |
| RDH5 | 2.5 | SLFN5 | 2.8 | VCL | 2.5.19 | ETHE1 | 2.8.41 |
| CILP2 | 2.5 | ATP2B1 | 2.8 | MOB3A | 2.5.19 | UBE2M | 2.8.41 |
| KAAG1 | 2.5 | TMPRSS2 | 2.8 | RHOB | 2.5.19 | CKLF | 2.8.41 |
| NDUFB8 | 2.5 | MBD2 | 2.8 | JUNB | 2.5.19 | TNFSF12 | 2.8.41 |
| CCDC3 | 2.5 | IQGAP2 | 2.8 | MT-ATP8 | 2.5.19 | AWAT1 | 2.8.41 |
| OR2AJ1 | 2.5 | ZC3H6 | 2.8 | JUN | 2.5.19 | LBX1 | 2.8.41 |
| PPP1R16A | 2.5 | MAX | 2.8 | CEBPD | 2.5.19 | UTP3 | 2.8.41 |
| EFNA4 | 2.5 | MBNL2 | 2.8 | PPP1R9B | 2.5.19 | WNT8B | 2.8.41 |
| ALPL | 2.5 | MRC1 | 2.8 | EHBP1L1 | 2.5.19 | DRGX | 2.8.41 |
| DUSP9 | 2.5 | DDX3X | 2.8 | PLS3 | 2.5.19 | ACTRT1 | 2.8.41 |
| RAC3 | 2.5 | THOC7 | 2.8 | CNPY3 | 2.5.19 | GPR61 | 2.8.41 |
| MYOC | 2.5 | PIP4K2A | 2.8 | FAM50A | 2.5.19 | TLR8 | 2.8.41 |
| STUB1 | 2.5 | NFKBIZ | 2.8 | PRDX5 | 2.5.19 | SEC14L4 | 2.8.41 |
| ENTPD2 | 2.5 | PTGES3 | 2.8 | CHMP1B | 2.5.19 | CCDC87 | 2.8.41 |
| C11orf52 | 2.5 | MFSD14A | 2.8 | KLF2 | 2.5.19 | PDZD7 | 2.8.41 |
| MT-CO3 | 2.5 | DIAPH1 | 2.8 | RBM15B | 2.5.19 | OR6M1 | 2.8.41 |
| OR4C3 | 2.5 | ARID5A | 2.8 | NPDC1 | 2.5.19 | ATP4A | 2.8.41 |
| IFNA1 | 2.5 | HMGN3 | 2.8 | MAP7D3 | 2.5.19 | OR10K1 | 2.8.41 |
| LZTS2 | 2.5 | POLE3 | 2.8 | KDELR3 | 2.5.19 | KRTAP13-2 | 2.8.41 |
| NEURL2 | 2.5 | LYN | 2.8 | TBX15 | 2.5.19 | ARMS2 | 2.8.41 |
| Clorf53 | 2.5 | LPXN | 2.8 | FPR1 | 2.5.19 | NDUFA8 | 2.8.41 |
| NR1D1 | 2.5 | RNF11 | 2.8 | BTBD2 | 2.5.19 | KTI12 | 2.8.41 |
| HSPB6 | 2.5 | AKAP3 | 2.8 | AURKAIP1 | 2.5.19 | FAM183A | 2.8.41 |
| LMAN1L | 2.5 | FCHSD2 | 2.8 | STMN3 | 2.5.19 | GPR62 | 2.8.41 |
| TMEM102 | 2.5 | AP2S1 | 2.8 | NDUFB7 | 2.5.19 | FGF19 | 2.8.41 |
| PTRHD1 | 2.5 | CCT4 | 2.8 | SOD3 | 2.5.19 | PCDHA7 | 2.8.41 |
| ADAM33 | 2.5 | MYDGF | 2.8 | UBXN6 | 2.5.19 | SMIM22 | 2.8.41 |
| FUT5 | 2.5 | KLK6 | 2.8 | SMOC2 | 2.5.19 | LGALS4 | 2.8.41 |
| ADRA2C | 2.5 | KCNK9 | 2.8 | MEPCE | 2.5.19 | MT1G | 2.8.41 |
| AATK | 2.5 | CA14 | 2.8 | NDN | 2.5.19 | IFNA13 | 2.8.41 |
| KLRC3 | 2.5 | GIMAP6 | 2.8 | CMTM3 | 2.5.19 | TEX29 | 2.8.41 |
| TELO2 | 2.5 | IRF1 | 2.8 | DBN1 | 2.5.19 | HOXC13 | 2.8.41 |
| COL12A1 | 2.5 | FER1L5 | 2.8 | TM4SF1 | 2.5.19 | OSM | 2.8.41 |
| CCDC105 | 2.5 | KNG1 | 2.8 | ITGB1BP1 | 2.5.19 | IQCF2 | 2.8.41 |
| HTRA3 | 2.5 | SCN5A | 2.8 | F5 | 2.5.19 | RSPH10B | 2.8.41 |
| NNAT | 2.5 | ETHE1 | 2.8 | PTGES2 | 2.5.19 | TNFSF13 | 2.8.41 |
| HOXD4 | 2.5 | TAGAP | 2.8 | SSNA1 | 2.5.19 | CCL16 | 2.8.41 |
| IRX5 | 2.5 | UBE2M | 2.8 | PDHA2 | 2.5.19 | SPATA45 | 2.8.41 |
| WFIKKN1 | 2.5 | CKLF | 2.8 | GOLGA8A | 2.5.19 | PNOC | 2.8.41 |
| SSC5D | 2.5 | TNFSF12 | 2.8 | TMEM205 | 2.5.19 | SOCS1 | 2.8.41 |
| TAC1 | 2.5 | AWAT1 | 2.8 | COA6 | 2.5.19 | SNURF | 2.8.41 |
| NOG | 2.5 | LBX1 | 2.8 | OR51S1 | 2.5.19 | ARHGDIB | 2.8.42 |
| KCNJ18 | 2.5 | UTP3 | 2.8 | RBFOX2 | 2.5.19 | EVI2B | 2.8.42 |
| SPON2 | 2.5 | WNT8B | 2.8 | MFAP2 | 2.5.19 | SRGN | 2.8.42 |
| MLN | 2.5 | DRGX | 2.8 | HES7 | 2.5.19 | PTPRC | 2.8.42 |
| SPATA2L | 2.5 | SERPINA5 | 2.8 | NSMF | 2.5.19 | LCP1 | 2.8.42 |
| JMJD7 | 2.5 | ACTRT1 | 2.8 | NR4A1 | 2.5.19 | PARP14 | 2.8.42 |
| OR5K2 | 2.5 | GPR61 | 2.8 | MALL | 2.5.19 | STAT1 | 2.8.42 |
| ADAM18 | 2.5 | TLR8 | 2.8 | OLFML2A | 2.5.19 | EML4 | 2.8.42 |
| MDK | 2.5 | SEC14L4 | 2.8 | MRI1 | 2.5.19 | SAMD9L | 2.8.42 |
| OR2T33 | 2.5 | CCDC87 | 2.8 | FBXO6 | 2.5.19 | APOL6 | 2.8.42 |
| HLA-DRB3 | 2.5 | PDZD7 | 2.8 | PRB2 | 2.5.19 | STARD7 | 2.8.42 |
| FBLN1 | 2.5 | OR6M1 | 2.8 | AMY2A | 2.5.19 | WIPF1 | 2.8.42 |
| ZNF521 | 2.5 | ATP4A | 2.8 | IL17D | 2.5.19 | CD53 | 2.8.42 |
| LRRC37A | 2.5 | OR10K1 | 2.8 | C1GALT1C1L | 2.5.19 | LYZ | 2.8.42 |
| OCLN | 2.5 | KRTAP13-2 | 2.8 | ALKBH7 | 2.5.19 | GBP1 | 2.8.42 |
| PPP1R14A | 2.5 | PCED1B | 2.8 | ADORA2A | 2.5.19 | GBP2 | 2.8.42 |
| CCDC125 | 2.5 | ARMS2 | 2.8 | PKM | 2.5.20 | CSF2RB | 2.8.42 |
| RPUSD1 | 2.5 | NDUFA8 | 2.8 | ALDOA | 2.5.20 | SMAP2 | 2.8.42 |
| CDH5 | 2.5 | GFRA3 | 2.8 | CTSD | 2.5.20 | LITAF | 2.8.42 |
| PAOX | 2.5 | KTI12 | 2.8 | EMP3 | 2.5.20 | DOK3 | 2.8.42 |
| RIMBP3 | 2.5 | FAM183A | 2.8 | ENO1 | 2.5.20 | SOD2 | 2.8.42 |
| OR9G1 | 2.5 | GPR32 | 2.8 | NDRG1 | 2.5.20 | IFI44L | 2.8.42 |
| MT-ND4 | 2.5 | GPR62 | 2.8 | AP1B1 | 2.5.20 | SNX10 | 2.8.42 |
| NCL | 2.5 | CCDC140 | 2.8 | ANPEP | 2.5.20 | CLEC2B | 2.8.42 |
| GAPDH | 2.5 | FGF19 | 2.8 | PFKP | 2.5.20 | LAP3 | 2.8.42 |
| SH3BGRL3 | 2.5 | PCDHA7 | 2.8 | PITPNA | 2.5.20 | RASSF2 | 2.8.42 |
| PFN1 | 2.5 | B3GNT6 | 2.8 | HK1 | 2.5.20 | SLC34A2 | 2.8.42 |
| SLC25A6 | 2.5 | KRTAP21-2 | 2.8 | CAPG | 2.5.20 | BAZIA | 2.8.42 |
| PRRX1 | 2.5 | MSMB | 2.8 | GOLM1 | 2.5.20 | TRIB1 | 2.8.42 |
| CALHM2 | 2.5 | GNAZ | 2.8 | PIP5K1C | 2.5.20 | IFI44 | 2.8.42 |
| GPX1 | 2.5 | RAB3A | 2.8 | CMKLR1 | 2.5.20 | CDKN1B | 2.8.42 |
| DDX5 | 2.5 | OTOP3 | 2.8 | PTGES | 2.5.20 | HCST | 2.8.42 |
| MAFB | 2.5 | SMIM22 | 2.8 | SFXN3 | 2.5.20 | TNFRSF1B | 2.8.42 |
| ABI3BP | 2.5 | ACER1 | 2.8 | GFPT2 | 2.5.20 | EXTL1 | 2.8.42 |
| SERPINB1 | 2.5 | LGALS4 | 2.8 | BLVRB | 2.5.20 | ARL4C | 2.8.42 |
| MYOF | 2.5 | MT1G | 2.8 | TSPAN15 | 2.5.20 | CST7 | 2.8.42 |
| TNFAIP2 | 2.5 | IFNA13 | 2.8 | MAPKAPK3 | 2.5.20 | IFIH1 | 2.8.42 |
| MCL1 | 2.5 | TEX29 | 2.8 | FABP3 | 2.5.20 | LYZL4 | 2.8.42 |
| MRC2 | 2.5 | HOXC13 | 2.8 | EIF3I | 2.5.20 | SOX18 | 2.8.42 |
| CYFIP1 | 2.5 | HAUS7 | 2.8 | CHI3L2 | 2.5.20 | SEMA5B | 2.8.42 |
| SNX9 | 2.5 | OSM | 2.8 | CDIPT | 2.5.20 | CXCL10 | 2.8.42 |
| TSPO | 2.5 | IQCF2 | 2.8 | UBACI | 2.5.20 | C15orf48 | 2.8.42 |
| VCL | 2.5 | RSPH10B | 2.8 | ARRDC1 | 2.5.20 | GZMA | 2.8.42 |
| NAV1 | 2.5 | TTC36 | 2.8 | GPER1 | 2.5.20 | GBP5 | 2.8.42 |
| MOB3A | 2.5 | TNFSF13 | 2.8 | EMILIN2 | 2.5.20 | IGSF6 | 2.8.42 |
| ADAP2 | 2.5 | CCL16 | 2.8 | VPS9D1 | 2.5.20 | GIMAP7 | 2.8.42 |
| RHOB | 2.5 | SPATA45 | 2.8 | KATNB1 | 2.5.20 | DNAI2 | 2.8.42 |
| JUNB | 2.5 | GOLGA6D | 2.8 | PGK1 | 2.5.20 | DTX3L | 2.8.42 |
| MT-ATP8 | 2.5 | PNOC | 2.8 | CA12 | 2.5.20 | ANKK1 | 2.8.42 |
| CDC42BPB | 2.5 | SOCS1 | 2.8 | TPRA1 | 2.5.20 | NAMPT | 2.8.42 |
| JUN | 2.5 | ZNHIT2 | 2.8 | GAS2L1 | 2.5.20 | FAM131C | 2.8.42 |
| OGDH | 2.5 | NT5C | 2.8 | TFEB | 2.5.20 | WSCD2 | 2.8.42 |
| CEBPD | 2.5 | SNURF | 2.8 | RAC3 | 2.5.20 | ACAN | 2.8.42 |
| FAT1 | 2.5 | CCL23 | 2.8 | HOXD4 | 2.5.20 | PCDHGB1 | 2.8.42 |
| PRKACA | 2.5 | TPP1 | 2.1 | MLN | 2.5.20 | RHOD | 2.8.42 |
| NFATC2 | 2.5 | LAPTM5 | 2.1 | RIMBP3 | 2.5.20 | IGLL1 | 2.8.42 |
| ZCCHC14 | 2.5 | TYROBP | 2.1 | OGDH | 2.5.20 | CSTA | 2.8.42 |
| DAG1 | 2.5 | CTSS | 2.1 | PRKACA | 2.5.20 | ZSCAN10 | 2.8.42 |
| LTBP2 | 2.5 | TMBIM6 | 2.1 | TUBB6 | 2.5.20 | LCP2 | 2.8.42 |
| ATP6VOB | 2.5 | CFL1 | 2.1 | NCSTN | 2.5.20 | IQGAP2 | 2.8.42 |
| TUBB6 | 2.5 | SIRPA | 2.1 | TRAPPC2L | 2.5.20 | PIP4K2A | 2.8.42 |
| PPP1R9B | 2.5 | UBE2L6 | 2.1 | SLC39A14 | 2.5.20 | LYN | 2.8.42 |
| PCDHGC3 | 2.5 | RPN2 | 2.1 | PCIF1 | 2.5.20 | FCHSD2 | 2.8.42 |
| TRIM47 | 2.5 | UCP2 | 2.1 | EFNB1 | 2.5.20 | CA14 | 2.8.42 |
| EHBP1L1 | 2.5 | GRB2 | 2.1 | MRPL24 | 2.5.20 | GIMAP6 | 2.8.42 |
| PLS3 | 2.5 | TMEM214 | 2.1 | NCK2 | 2.5.20 | IRF1 | 2.8.42 |
| NCSTN | 2.5 | SLCO2B1 | 2.1 | ODF3B | 2.5.20 | TAGAP | 2.8.42 |
| CTTN | 2.5 | ERGIC3 | 2.1 | LOXL2 | 2.5.20 | SERPINA5 | 2.8.42 |
| CNPY3 | 2.5 | ARRB2 | 2.1 | PET117 | 2.5.20 | PCED1B | 2.8.42 |
| FAM50A | 2.5 | FCER1G | 2.1 | OR4L1 | 2.5.20 | GFRA3 | 2.8.42 |
| PLPP3 | 2.5 | SH2B3 | 2.1 | TSTD1 | 2.5.20 | GPR32 | 2.8.42 |
| PRDX5 | 2.5 | AUP1 | 2.1 | HSPBP1 | 2.5.20 | CCDC140 | 2.8.42 |
| BCL6 | 2.5 | MAN2B1 | 2.1 | RNF103-CHMP3 | 2.5.20 | B3GNT6 | 2.8.42 |
| ATP8B2 | 2.5 | TPM3 | 2.1 | CIR | 2.5.21 | KRTAP21-2 | 2.8.42 |
| CYTH3 | 2.5 | SIDT2 | 2.1 | CTSB | 2.5.21 | MSMB | 2.8.42 |
| FGD5 | 2.5 | TIMP1 | 2.1 | GRN | 2.5.21 | ACER1 | 2.8.42 |
| F13A1 | 2.5 | SYNGR2 | 2.1 | C1QC | 2.5.21 | NT5C | 2.8.42 |
| CHMP1B | 2.5 | COPG1 | 2.1 | CSF1R | 2.5.21 | CCL23 | 2.8.42 |
| FBLN2 | 2.5 | SPCS1 | 2.1 | CD74 | 2.5.21 | ALAS2 | 2.9.45 |
| HDAC7 | 2.5 | ZNF385A | 2.1 | PLTP | 2.5.21 | SLC4A1 | 2.9.45 |
| UFC1 | 2.5 | ARPC1B | 2.1 | MMP2 | 2.5.21 | HBD | 2.9.45 |
| IGBP1 | 2.5 | SDC3 | 2.1 | CTSA | 2.5.21 | TUBB1 | 2.9.45 |
| STX11 | 2.5 | PTPN18 | 2.1 | FCGR2A | 2.5.21 | TNFRSF10C | 2.9.45 |
| PACS2 | 2.5 | SARAF | 2.1 | CCDC80 | 2.5.21 | DMTN | 2.9.45 |
| TRAPPC2L | 2.5 | TMEM176B | 2.1 | PLBD2 | 2.5.21 | CXCR2 | 2.9.45 |
| KLF2 | 2.5 | CD4 | 2.1 | RNASE1 | 2.5.21 | CXCR1 | 2.9.45 |
| GAS1 | 2.5 | GMFG | 2.1 | GLUL | 2.5.21 | PI3 | 2.9.45 |
| RBM15B | 2.5 | LGALS3BP | 2.1 | CST3 | 2.5.21 | CLC | 2.9.45 |
| NPDC1 | 2.5 | SYVN1 | 2.1 | IGFBP4 | 2.5.21 | HBQ1 | 2.9.45 |
| SLC39A14 | 2.5 | PTPN1 | 2.1 | SPI1 | 2.5.21 | MPND | 2.9.45 |
| MAP7D3 | 2.5 | CTBP1 | 2.1 | PLA2G2A | 2.5.21 | SPRR2A | 2.9.45 |
| KDELR3 | 2.5 | C11orf24 | 2.1 | SELPLG | 2.5.21 | CD24 | 2.9.45 |
| MVP | 2.5 | PSME2 | 2.1 | CD14 | 2.5.21 | S100P | 2.9.45 |
| MYC | 2.5 | TIMD4 | 2.1 | C1S | 2.5.21 | CMTM2 | 2.9.45 |
| TBX15 | 2.5 | ST3GAL1 | 2.1 | TNFRSF1A | 2.5.21 | NFE2 | 2.9.45 |
| FPR1 | 2.5 | PPIB | 2.1 | CD58 | 2.5.21 | S100A12 | 2.9.45 |
| BTBD2 | 2.5 | TMEM127 | 2.1 | MYD88 | 2.5.21 | TES | 2.9.45 |
| AURKAIP1 | 2.5 | GORASP2 | 2.1 | CTSZ | 2.5.21 | GP9 | 2.9.45 |
| MAP3K6 | 2.5 | IL1ORA | 2.1 | MARCO | 2.5.21 | PDZK1IP1 | 2.9.45 |
| STMN3 | 2.5 | CYBA | 2.1 | SCARA5 | 2.5.21 | HBM | 2.9.45 |
| NDUFB7 | 2.5 | DOK2 | 2.1 | GPR137B | 2.5.21 | TPP1 | 2.10.46 |
| SOD3 | 2.5 | UBE2Q1 | 2.1 | NNMT | 2.5.21 | LAPTM5 | 2.10.46 |
| UBXN6 | 2.5 | GAA | 2.1 | TUFM | 2.5.21 | CTSS | 2.10.46 |
| ANO1 | 2.5 | NEK6 | 2.1 | TTYH3 | 2.5.21 | TMBIM6 | 2.10.46 |
| SMOC2 | 2.5 | RAB8A | 2.1 | SHARPIN | 2.5.21 | CFL1 | 2.10.46 |
| FAM89B | 2.5 | CXCL16 | 2.1 | TMEM119 | 2.5.21 | UBE2L6 | 2.10.46 |
| RBM10 | 2.5 | APOBEC3C | 2.1 | SLC25A5 | 2.5.21 | RPN2 | 2.10.46 |
| MEPCE | 2.5 | ARAF | 2.1 | GNB2 | 2.5.21 | UCP2 | 2.10.46 |
| MMP19 | 2.5 | CNN2 | 2.1 | WDR45B | 2.5.21 | GRB2 | 2.10.46 |
| TSPYL2 | 2.5 | TYMP | 2.1 | CHI3L1 | 2.5.21 | SLCO2B1 | 2.10.46 |
| TBC1D2 | 2.5 | CD84 | 2.1 | ADRMI | 2.5.21 | ERGIC3 | 2.10.46 |
| TUBA1B | 2.5 | ACP2 | 2.1 | IL13RA1 | 2.5.21 | ARRB2 | 2.10.46 |
| YIPF2 | 2.5 | RPN1 | 2.1 | UAP1 | 2.5.21 | MAN2B1 | 2.10.46 |
| THBS4 | 2.5 | RHOG | 2.1 | CIQA | 2.5.21 | TPM3 | 2.10.46 |
| NDN | 2.5 | RNF114 | 2.1 | SERPINF1 | 2.5.21 | TIMP1 | 2.10.46 |
| PCIF1 | 2.5 | C5AR1 | 2.1 | TSPAN4 | 2.5.21 | SYNGR2 | 2.10.46 |
| CMTM3 | 2.5 | GATAD2A | 2.1 | HLA-DRB1 | 2.5.21 | COPG1 | 2.10.46 |
| DBN1 | 2.5 | SUMF2 | 2.1 | IRX5 | 2.5.21 | SPCS1 | 2.10.46 |
| TM4SF1 | 2.5 | SSU72 | 2.1 | TAC1 | 2.5.21 | ZNF385A | 2.10.46 |
| TRIP10 | 2.5 | B2M | 2.1 | MDK | 2.5.21 | ARPC1B | 2.10.46 |
| ITGA11 | 2.5 | ATP6VOD1 | 2.1 | PFN1 | 2.5.21 | SARAF | 2.10.46 |
| EFNB1 | 2.5 | PDIA4 | 2.1 | MAFB | 2.5.21 | CD4 | 2.10.46 |
| ITGB1BP1 | 2.5 | ERP29 | 2.1 | SERPINB1 | 2.5.21 | GMFG | 2.10.46 |
| F5 | 2.5 | ZYX | 2.1 | TNFAIP2 | 2.5.21 | LGALS3BP | 2.10.46 |
| PTGES2 | 2.5 | PRKCD | 2.1 | CYFIP1 | 2.5.21 | CTBP1 | 2.10.46 |
| SSNA1 | 2.5 | SECTM1 | 2.1 | ATP6VOB | 2.5.21 | C11orf24 | 2.10.46 |
| FOXI3 | 2.5 | TREM1 | 2.1 | F13A1 | 2.5.21 | PPIB | 2.10.46 |
| NCKIPSD | 2.5 | NOP56 | 2.1 | IGBP1 | 2.5.21 | GORASP2 | 2.10.46 |
| GSX2 | 2.5 | NAXE | 2.1 | STX11 | 2.5.21 | IL10RA | 2.10.46 |
| KPNA7 | 2.5 | TMEM106A | 2.1 | TBC1D2 | 2.5.21 | CYBA | 2.10.46 |
| PXDC1 | 2.5 | IL18BP | 2.1 | YIPF2 | 2.5.21 | DOK2 | 2.10.46 |
| PDHA2 | 2.5 | HM13 | 2.1 | EIF4EBP1 | 2.5.21 | GAA | 2.10.46 |
| MRPL24 | 2.5 | RHOC | 2.1 | EGR4 | 2.5.21 | NEK6 | 2.10.46 |
| NT5C3B | 2.5 | ACTRIB | 2.1 | FGF10 | 2.5.21 | APOBEC3C | 2.10.46 |
| NCK2 | 2.5 | AKR1A1 | 2.1 | ASCL2 | 2.5.21 | ARAF | 2.10.46 |
| EIF4EBP1 | 2.5 | CHST15 | 2.1 | CDH15 | 2.5.21 | CNN2 | 2.10.46 |
| STK11 | 2.5 | JTB | 2.1 | FNDC10 | 2.5.21 | TYMP | 2.10.46 |
| GOLGA8A | 2.5 | HGS | 2.1 | WDR18 | 2.5.21 | RPN1 | 2.10.46 |
| TLR7 | 2.5 | CADM3 | 2.1 | SAA1 | 2.5.21 | RNF114 | 2.10.46 |
| ODF3B | 2.5 | MMP3 | 2.1 | DPYSL2 | 2.5.23 | C5AR1 | 2.10.46 |
| TMEM205 | 2.5 | ACP5 | 2.1 | NOTCH2 | 2.5.23 | SSU72 | 2.10.46 |
| FUCA2 | 2.5 | IMPDH2 | 2.1 | ATP2B4 | 2.5.23 | B2M | 2.10.46 |
| LOXL2 | 2.5 | GZMB | 2.1 | NFIC | 2.5.23 | PDIA4 | 2.10.46 |
| OR7G1 | 2.5 | DNAJC5 | 2.1 | COLEC12 | 2.5.23 | ERP29 | 2.10.46 |
| HES1 | 2.5 | UNC5B | 2.1 | TACC1 | 2.5.23 | ZYX | 2.10.46 |
| COA6 | 2.5 | SPSB1 | 2.1 | TMEM43 | 2.5.23 | PRKCD | 2.10.46 |
| OR51S1 | 2.5 | MANF | 2.1 | DPYSL3 | 2.5.23 | SECTM1 | 2.10.46 |
| PET117 | 2.5 | RUVBL2 | 2.1 | TNS1 | 2.5.23 | RHOC | 2.10.46 |
| SUSD2 | 2.5 | NECAP2 | 2.1 | ABHD2 | 2.5.23 | AKR1A1 | 2.10.46 |
| MLST8 | 2.5 | YIF1A | 2.1 | AXL | 2.5.23 | CHST15 | 2.10.46 |
| ESAM | 2.5 | SIGMAR1 | 2.1 | EMP1 | 2.5.23 | GZMB | 2.10.46 |
| EGR4 | 2.5 | TCF3 | 2.1 | SKI | 2.5.23 | PSMD7 | 2.10.46 |
| SHCBP1L | 2.5 | TRAM2 | 2.1 | LTBP3 | 2.5.23 | ATP6V1G1 | 2.10.46 |
| TMEM160 | 2.5 | HMOX1 | 2.1 | ESYT2 | 2.5.23 | B4GALT1 | 2.10.46 |
| PTGIS | 2.5 | NDUFA11 | 2.1 | AEBP1 | 2.5.23 | EIF3G | 2.10.46 |
| RBFOX2 | 2.5 | DLX3 | 2.1 | ANGPTL2 | 2.5.23 | LSM7 | 2.10.46 |
| APLNR | 2.5 | SLC16A3 | 2.1 | PMEPA1 | 2.5.23 | METTL24 | 2.10.46 |
| KCTD11 | 2.5 | ZBED1 | 2.1 | MAP4 | 2.5.23 | RAB11B | 2.10.46 |
| FAM180B | 2.5 | DUSP3 | 2.1 | SCARA3 | 2.5.23 | TBCC | 2.10.46 |
| MFAP2 | 2.5 | CSK | 2.1 | HSPG2 | 2.5.23 | HCK | 2.10.46 |
| FASN | 2.5 | LYNX1 | 2.1 | ACKR3 | 2.5.23 | TXNDC12 | 2.10.46 |
| HES7 | 2.5 | SMPD1 | 2.1 | CAMK1D | 2.5.23 | PTGDS | 2.10.46 |
| NSMF | 2.5 | G6PD | 2.1 | CLEC3B | 2.5.23 | PSME1 | 2.10.46 |
| OR6Y1 | 2.5 | SF3A1 | 2.1 | FGFR1 | 2.5.23 | CXCR3 | 2.10.46 |
| APOD | 2.5 | FXYD6 | 2.1 | GPX3 | 2.5.23 | AGR3 | 2.10.46 |
| ACOT7 | 2.5 | MMP1 | 2.1 | DPT | 2.5.23 | OR6C76 | 2.10.46 |
| FGF10 | 2.5 | DDOST | 2.1 | AKAP13 | 2.5.23 | NRROS | 2.10.46 |
| ASCL2 | 2.5 | POLR2G | 2.1 | RAB1B | 2.5.23 | CHST12 | 2.10.46 |
| SPON1 | 2.5 | RARRES1 | 2.1 | CERCAM | 2.5.23 | R3HCC1 | 2.10.46 |
| ALKBH4 | 2.5 | DGAT1 | 2.1 | KANK2 | 2.5.23 | ST6GALNAC4 | 2.10.46 |
| ARL2BP | 2.5 | SLC2A6 | 2.1 | THBS3 | 2.5.23 | YDJC | 2.10.46 |
| OR4L1 | 2.5 | PSMD7 | 2.1 | SORBS3 | 2.5.23 | ATAD3A | 2.10.46 |
| CDH15 | 2.5 | ATP6V1G1 | 2.1 | PER2 | 2.5.23 | SNRPB | 2.10.46 |
| NR4A1 | 2.5 | B4GALT1 | 2.1 | HIVEP2 | 2.5.23 | SDR39U1 | 2.10.46 |
| MALL | 2.5 | PARP3 | 2.1 | CAPN2 | 2.5.23 | DEFA5 | 2.10.46 |
| OLFML2A | 2.5 | EIF3G | 2.1 | LRRC15 | 2.5.23 | SYF2 | 2.10.46 |
| FAM47B | 2.5 | LPAR5 | 2.1 | GAS7 | 2.5.23 | TSSC4 | 2.10.46 |
| XKR8 | 2.5 | PLOD3 | 2.1 | KLF4 | 2.5.23 | FOXD3 | 2.10.46 |
| ADRA2A | 2.5 | CTSF | 2.1 | TPPP | 2.5.23 | FAM78A | 2.10.46 |
| REM1 | 2.5 | TICAM1 | 2.1 | KLF9 | 2.5.23 | ASAHI | 2.10.46 |
| SLN | 2.5 | TRPV2 | 2.1 | ZNF503 | 2.5.23 | TINF2 | 2.10.46 |
| TSTD1 | 2.5 | GPAA1 | 2.1 | ZNF768 | 2.5.23 | SCAMP2 | 2.10.46 |
| FNDC10 | 2.5 | CDC42EP1 | 2.1 | PTPRS | 2.5.23 | STX5 | 2.10.46 |
| EIF4EBP3 | 2.5 | MPDU1 | 2.1 | KDELR2 | 2.5.23 | SRPRA | 2.10.46 |
| MRI1 | 2.5 | LSM7 | 2.1 | SLC12A4 | 2.5.23 | GSTK1 | 2.10.46 |
| TMUB1 | 2.5 | MAD2L2 | 2.1 | ABL1 | 2.5.23 | RIC8A | 2.10.46 |
| WDR18 | 2.5 | ZSWIM1 | 2.1 | RGCC | 2.5.23 | MLEC | 2.10.46 |
| FBXO6 | 2.5 | CHST2 | 2.1 | SLC39A13 | 2.5.23 | SH3KBP1 | 2.10.46 |
| ATOH7 | 2.5 | METTL24 | 2.1 | CDH23 | 2.5.23 | MPP1 | 2.10.46 |
| HSPBP1 | 2.5 | ERGIC1 | 2.1 | PER1 | 2.5.23 | MLKL | 2.10.46 |
| OR10A5 | 2.5 | RAB11B | 2.1 | MRFAP1 | 2.5.23 | HSF1 | 2.10.46 |
| PRB2 | 2.5 | FBP1 | 2.1 | NDUFA4L2 | 2.5.23 | MAGED1 | 2.10.46 |
| AMY2A | 2.5 | TMEM176A | 2.1 | MAMDC2 | 2.5.23 | CMTM7 | 2.10.46 |
| ACTRT2 | 2.5 | LFNG | 2.1 | COL8A2 | 2.5.23 | MRPL34 | 2.10.46 |
| SH2D1A | 2.5 | TMEM51 | 2.1 | MAP1A | 2.5.23 | SLBP | 2.10.46 |
| IL17D | 2.5 | DNPH1 | 2.1 | OR1J4 | 2.5.23 | SMCR8 | 2.10.46 |
| CSN2 | 2.5 | FZR1 | 2.1 | PTTG2 | 2.5.23 | LRRIQ4 | 2.10.46 |
| C1GALT1CIL | 2.5 | CHST11 | 2.1 | SERPINE1 | 2.5.23 | NAPA | 2.10.46 |
| NOXA1 | 2.5 | STEAP3 | 2.1 | TSKU | 2.5.23 | GRAMDIA | 2.10.46 |
| NGFR | 2.5 | TBCC | 2.1 | TNS2 | 2.5.23 | PPP4C | 2.10.46 |
| FCN2 | 2.5 | TMEM37 | 2.1 | FZD10 | 2.5.23 | ECHS1 | 2.10.46 |
| SUV39H1 | 2.5 | IL32 | 2.1 | DUSP9 | 2.5.23 | BANF1 | 2.10.46 |
| C1QL1 | 2.5 | CD300C | 2.1 | NR1D1 | 2.5.23 | KRTAP19-5 | 2.10.46 |
| LYZL2 | 2.5 | MCRS1 | 2.1 | TMEM102 | 2.5.23 | MIEN1 | 2.10.46 |
| RGPD8 | 2.5 | HCK | 2.1 | SSC5D | 2.5.23 | KRTAP20-2 | 2.10.46 |
| SAA1 | 2.5 | TXNDC12 | 2.1 | HLA-DRB3 | 2.5.23 | LSM10 | 2.10.46 |
| PHOX2A | 2.5 | DNAJC4 | 2.1 | MYOF | 2.5.23 | UTS2 | 2.10.46 |
| ALKBH7 | 2.5 | PTGDS | 2.1 | SNX9 | 2.5.23 | SCGB3A2 | 2.10.46 |
| RNF103-CHMP3 | 2.5 | GANAB | 2.1 | NAV1 | 2.5.23 | RRS1 | 2.10.46 |
| ADORA2A | 2.5 | PSME1 | 2.1 | CDC42BPB | 2.5.23 | ARL14 | 2.10.46 |
| TRIM61 | 2.5 | CXCR3 | 2.1 | NFATC2 | 2.5.23 | KIF26B | 2.10.46 |
| TCP11X2 | 2.5 | C10orf105 | 2.1 | ZCCHC14 | 2.5.23 | SIRPA | 2.10.48 |
| LRRC37A2 | 2.5 | AGR3 | 2.1 | DAG1 | 2.5.23 | TMEM214 | 2.10.48 |
| ZNF32 | 2.5 | OR6C76 | 2.1 | LTBP2 | 2.5.23 | SH2B3 | 2.10.48 |
| FOXE3 | 2.5 | BTBD6 | 2.1 | PCDHGC3 | 2.5.23 | AUP1 | 2.10.48 |
| RPS11 | 2.6 | NRROS | 2.1 | TRIM47 | 2.5.23 | SIDT2 | 2.10.48 |
| RPL11 | 2.6 | GNA15 | 2.1 | CTTN | 2.5.23 | PTPN18 | 2.10.48 |
| RPL31 | 2.6 | NXT1 | 2.1 | ATP8B2 | 2.5.23 | SYVN1 | 2.10.48 |
| RPS25 | 2.6 | CHST12 | 2.1 | FGD5 | 2.5.23 | TMEM127 | 2.10.48 |
| RPS23 | 2.6 | FAM167B | 2.1 | ANO1 | 2.5.23 | UBE2Q1 | 2.10.48 |
| RPL32 | 2.6 | R3HCC1 | 2.1 | FAM89B | 2.5.23 | CXCL16 | 2.10.48 |
| RPS20 | 2.6 | ST6GALNAC4 | 2.1 | THBS4 | 2.5.23 | GATAD2A | 2.10.48 |
| RPS16 | 2.6 | RNF26 | 2.1 | PXDC1 | 2.5.23 | SUMF2 | 2.10.48 |
| RPS21 | 2.6 | ATP13A2 | 2.1 | NT5C3B | 2.5.23 | NAXE | 2.10.48 |
| RACK1 | 2.6 | YDJC | 2.1 | HES1 | 2.5.23 | TMEM106A | 2.10.48 |
| RPL34 | 2.6 | ATAD3A | 2.1 | SHCBP1L | 2.5.23 | HM13 | 2.10.48 |
| RPL19 | 2.6 | SNRPB | 2.1 | TMEM160 | 2.5.23 | JTB | 2.10.48 |
| RPL38 | 2.6 | AGTRAP | 2.1 | OR6Y1 | 2.5.23 | CADM3 | 2.10.48 |
| RPL27 | 2.6 | CLDN34 | 2.1 | ALKBH4 | 2.5.23 | IMPDH2 | 2.10.48 |
| RPL23 | 2.6 | LSM4 | 2.1 | ARL2BP | 2.5.23 | SPSB1 | 2.10.48 |
| UBA52 | 2.6 | FIS1 | 2.1 | XKR8 | 2.5.23 | RUVBL2 | 2.10.48 |
| TPT1 | 2.6 | ICAM1 | 2.1 | SLN | 2.5.23 | SIGMAR1 | 2.10.48 |
| RPS6 | 2.6 | GJB2 | 2.1 | OR10A5 | 2.5.23 | TCF3 | 2.10.48 |
| RPL35A | 2.6 | SLC43A2 | 2.1 | PLEKHM2 | 2.5.24 | TRAM2 | 2.10.48 |
| RPL4 | 2.6 | ZFPL1 | 2.1 | SEC61A1 | 2.5.24 | NDUFA11 | 2.10.48 |
| RPS8 | 2.6 | OR51A2 | 2.1 | COLGALT1 | 2.5.24 | SLC16A3 | 2.10.48 |
| RPL10A | 2.6 | SDR39U1 | 2.1 | TBC1D14 | 2.5.24 | ZBED1 | 2.10.48 |
| BTF3 | 2.6 | SMIM3 | 2.1 | MAPKAPK2 | 2.5.24 | DUSP3 | 2.10.48 |
| RPL15 | 2.6 | RERGL | 2.1 | APEX1 | 2.5.24 | G6PD | 2.10.48 |
| RPS12 | 2.6 | ABHD15 | 2.1 | SZRD1 | 2.5.24 | DDOST | 2.10.48 |
| RPL18 | 2.6 | AKAIN1 | 2.1 | BET1L | 2.5.24 | POLR2G | 2.10.48 |
| RPL36 | 2.6 | DEFA5 | 2.1 | QRICH1 | 2.5.24 | DGAT1 | 2.10.48 |
| RPL3 | 2.6 | RGPD2 | 2.1 | CCDC71 | 2.5.24 | PARP3 | 2.10.48 |
| RPS13 | 2.6 | SYF2 | 2.1 | ARPC4 | 2.5.24 | CTSF | 2.10.48 |
| HEG1 | 2.6 | BAAT | 2.1 | ADAMTSL4 | 2.5.24 | GPAA1 | 2.10.48 |
| RPLP2 | 2.6 | TSSC4 | 2.1 | THY1 | 2.5.24 | CDC42EP1 | 2.10.48 |
| COX6B1 | 2.6 | KCNQ1 | 2.1 | TRAPPC1 | 2.5.24 | ZSWIM1 | 2.10.48 |
| RPL13A | 2.6 | TRMT2A | 2.1 | STAT3 | 2.5.24 | CHST2 | 2.10.48 |
| UBB | 2.6 | FAM20A | 2.1 | ARL2 | 2.5.24 | ERGIC1 | 2.10.48 |
| ATP6VOE1 | 2.6 | POTEH | 2.1 | GJA1 | 2.5.24 | LFNG | 2.10.48 |
| RPS3 | 2.6 | CEMIP | 2.1 | TTC7A | 2.5.24 | DNPH1 | 2.10.48 |
| RPL37A | 2.6 | MTHFS | 2.1 | CHID1 | 2.5.24 | DNAJC4 | 2.10.48 |
| UQCRQ | 2.6 | TNFAIP8L2 | 2.1 | ADCY7 | 2.5.24 | C10orf105 | 2.10.48 |
| NDUFS5 | 2.6 | CDRT15 | 2.1 | PLXNA3 | 2.5.24 | NXT1 | 2.10.48 |
| NACA | 2.6 | ARC | 2.1 | MED16 | 2.5.24 | RNF26 | 2.10.48 |
| EIF3L | 2.6 | CEBPA | 2.1 | ALOX5AP | 2.5.24 | CLDN34 | 2.10.48 |
| SHISA5 | 2.6 | GHRH | 2.1 | GUCD1 | 2.5.24 | FIS1 | 2.10.48 |
| RPL24 | 2.6 | HTRA2 | 2.1 | LSS | 2.5.24 | ICAM1 | 2.10.48 |
| CD59 | 2.6 | FOXD3 | 2.1 | NBPF10 | 2.5.24 | RGPD2 | 2.10.48 |
| PRDX1 | 2.6 | HLA-C | 2.1 | TUBGCP6 | 2.5.24 | BAAT | 2.10.48 |
| NDUFA1 | 2.6 | FAM78A | 2.1 | CDT1 | 2.5.24 | KCNQ1 | 2.10.48 |
| RPL14 | 2.6 | MPG | 2.1 | SLC52A2 | 2.5.24 | TRMT2A | 2.10.48 |
| COX7A2 | 2.6 | ASAHI | 2.1 | CPXM1 | 2.5.24 | CEBPA | 2.10.48 |
| CRTAP | 2.6 | TINF2 | 2.1 | FUT5 | 2.5.24 | MPG | 2.10.48 |
| RPS2 | 2.6 | SCAMP2 | 2.1 | AATK | 2.5.24 | ITGA5 | 2.10.48 |
| RPS24 | 2.6 | STX5 | 2.1 | HTRA3 | 2.5.24 | TCTA | 2.10.48 |
| NDUFA12 | 2.6 | SRPRA | 2.1 | SPATA2L | 2.5.24 | UBE2I | 2.10.48 |
| UBL5 | 2.6 | GSTK1 | 2.1 | ADAM18 | 2.5.24 | PSMB4 | 2.10.48 |
| FAU | 2.6 | ITGA5 | 2.1 | RPUSD1 | 2.5.24 | ZNF423 | 2.10.48 |
| RPLP1 | 2.6 | RIC8A | 2.1 | UFC1 | 2.5.24 | SLC25A28 | 2.10.48 |
| HINT1 | 2.6 | TCTA | 2.1 | MVP | 2.5.24 | UBTF | 2.10.48 |
| EIF3K | 2.6 | ITGAM | 2.1 | RBM10 | 2.5.24 | PSMC4 | 2.10.48 |
| DAD1 | 2.6 | MLEC | 2.1 | ESAM | 2.5.24 | MRPL17 | 2.10.48 |
| TMEM50A | 2.6 | SH3KBP1 | 2.1 | ATOH7 | 2.5.24 | RER1 | 2.10.48 |
| VAMP5 | 2.6 | MPP1 | 2.1 | LYZL2 | 2.5.24 | REPIN1 | 2.10.48 |
| SLC25A37 | 2.6 | UBE2I | 2.1 | TRIM61 | 2.5.24 | CD209 | 2.10.48 |
| PFDN5 | 2.6 | RAB32 | 2.1 | ZNF32 | 2.5.24 | FARSA | 2.10.48 |
| POLR2L | 2.6 | MLKL | 2.1 | DCN | 2.5.25 | ZNF205 | 2.10.48 |
| RPL36AL | 2.6 | PSMB4 | 2.1 | PRELP | 2.5.25 | DEFB121 | 2.10.48 |
| OST4 | 2.6 | GSTO1 | 2.1 | ASPN | 2.5.25 | Clorf54 | 2.10.48 |
| MYL12B | 2.6 | ZNF423 | 2.1 | CYBRD1 | 2.5.25 | SIX5 | 2.10.48 |
| NDUFB1 | 2.6 | HSF1 | 2.1 | AQP1 | 2.5.25 | SDC3 | 2.10.49 |
| SULF2 | 2.6 | SLC25A28 | 2.1 | MGP | 2.5.25 | TMEM176B | 2.10.49 |
| RPL35 | 2.6 | MAGED1 | 2.1 | COL14A1 | 2.5.25 | PTPN1 | 2.10.49 |
| SLIRP | 2.6 | CMTM7 | 2.1 | COMP | 2.5.25 | TIMD4 | 2.10.49 |
| DYNLRB1 | 2.6 | MRPL34 | 2.1 | TGFBR3 | 2.5.25 | CD84 | 2.10.49 |
| LPAR1 | 2.6 | SLBP | 2.1 | FMOD | 2.5.25 | ACP2 | 2.10.49 |
| SEC61B | 2.6 | SMCR8 | 2.1 | PCOLCE2 | 2.5.25 | TREM1 | 2.10.49 |
| RPS19 | 2.6 | NAGA | 2.1 | POSTN | 2.5.25 | MMP3 | 2.10.49 |
| C12orf57 | 2.6 | UBTF | 2.1 | CRLF1 | 2.5.25 | DLX3 | 2.10.49 |
| POLR2K | 2.6 | PSMC4 | 2.1 | LYVE1 | 2.5.25 | LYNX1 | 2.10.49 |
| RPS27A | 2.6 | LRRIQ4 | 2.1 | NFIA | 2.5.25 | FXYD6 | 2.10.49 |
| GYPC | 2.6 | RNPEP | 2.1 | GALNT15 | 2.5.25 | MMP1 | 2.10.49 |
| PFDN2 | 2.6 | MRPL17 | 2.1 | CXCL14 | 2.5.25 | SLC2A6 | 2.10.49 |
| RPS15 | 2.6 | NAPA | 2.1 | DKK3 | 2.5.25 | TRPV2 | 2.10.49 |
| CWC15 | 2.6 | RER1 | 2.1 | IGF1 | 2.5.25 | TMEM176A | 2.10.49 |
| RPL5 | 2.6 | GLMP | 2.1 | SFRP2 | 2.5.25 | STEAP3 | 2.10.49 |
| SSR4 | 2.6 | MAPK7 | 2.1 | CILP | 2.5.25 | TMEM37 | 2.10.49 |
| SLC25A39 | 2.6 | FERMT3 | 2.1 | SFRP4 | 2.5.25 | CD300C | 2.10.49 |
| RPL13 | 2.6 | REPIN1 | 2.1 | CNN3 | 2.5.25 | GANAB | 2.10.49 |
| SUPT4H1 | 2.6 | GRAMDIA | 2.1 | NTN1 | 2.5.25 | GNA15 | 2.10.49 |
| EIF3H | 2.6 | GPR183 | 2.1 | MFAP5 | 2.5.25 | LSM4 | 2.10.49 |
| COX7B | 2.6 | PPP4C | 2.1 | FAM205A | 2.5.25 | ZFPL1 | 2.10.49 |
| RPL39 | 2.6 | ECHS1 | 2.1 | RDH5 | 2.5.25 | OR51A2 | 2.10.49 |
| FAM32A | 2.6 | CD209 | 2.1 | MYOC | 2.5.25 | FAM20A | 2.10.49 |
| COX6C | 2.6 | BANF1 | 2.1 | OR4C3 | 2.5.25 | TNFAIP8L2 | 2.10.49 |
| CNBP | 2.6 | KRTAP19-5 | 2.1 | HSPB6 | 2.5.25 | ARC | 2.10.49 |
| MT1E | 2.6 | FARSA | 2.1 | COL12A1 | 2.5.25 | HLA-C | 2.10.49 |
| NRGN | 2.6 | DNASE2 | 2.1 | KCNJ18 | 2.5.25 | ITGAM | 2.10.49 |
| RPL27A | 2.6 | ALAS1 | 2.1 | ZNF521 | 2.5.25 | NAGA | 2.10.49 |
| IDH2 | 2.6 | MPV17L2 | 2.1 | PRRX1 | 2.5.25 | GLMP | 2.10.49 |
| VPS29 | 2.6 | MIEN1 | 2.1 | PLPP3 | 2.5.25 | MAPK7 | 2.10.49 |
| VAMP8 | 2.6 | KRTAP20-2 | 2.1 | FBLN2 | 2.5.25 | IFNA8 | 2.10.49 |
| TMUB2 | 2.6 | GAL3ST4 | 2.1 | GAS1 | 2.5.25 | TOR4A | 2.10.49 |
| RPL30 | 2.6 | NUBP1 | 2.1 | ITGA11 | 2.5.25 | MRPL54 | 2.10.49 |
| EDF1 | 2.6 | LSM10 | 2.1 | PTGIS | 2.5.25 | ADAR | 2.12.51 |
| RPS4X | 2.6 | ZNF205 | 2.1 | FAM180B | 2.5.25 | RNF213 | 2.12.51 |
| COA3 | 2.6 | DEFB121 | 2.1 | SPON1 | 2.5.25 | MYO9B | 2.12.51 |
| RPL37 | 2.6 | UTS2 | 2.1 | ADRA2A | 2.5.25 | MPEG1 | 2.12.51 |
| PHB2 | 2.6 | IFNA8 | 2.1 | EIF4EBP3 | 2.5.25 | EIF4G1 | 2.12.51 |
| MAPK3 | 2.6 | SCGB3A2 | 2.1 | ACTRT2 | 2.5.25 | RALGDS | 2.12.51 |
| FKBP10 | 2.6 | MIF4GD | 2.1 | FCN2 | 2.5.25 | NMT1 | 2.12.51 |
| RPLPO | 2.6 | UAP1L1 | 2.1 | SUV39H1 | 2.5.25 | IFI6 | 2.12.51 |
| COX7C | 2.6 | RRS1 | 2.1 | RGPD8 | 2.5.25 | ILF3 | 2.12.51 |
| TOB2 | 2.6 | TOR4A | 2.1 | C1QB | 2.5.26 | RAPGEF1 | 2.12.51 |
| COX8A | 2.6 | ARL14 | 2.1 | VSIG4 | 2.5.26 | ETV6 | 2.12.51 |
| RPL7A | 2.6 | MRPL54 | 2.1 | FPR3 | 2.5.26 | RCC2 | 2.12.51 |
| COX14 | 2.6 | Clorf54 | 2.1 | FOLR2 | 2.5.26 | RASA3 | 2.12.51 |
| TMEM258 | 2.6 | SPR | 2.1 | CTSL | 2.5.26 | ADGRE5 | 2.12.51 |
| UQCR10 | 2.6 | SIX5 | 2.1 | C3AR1 | 2.5.26 | SLC9A3R1 | 2.12.51 |
| RPL18A | 2.6 | GTF2H2 | 2.1 | MS4A6A | 2.5.26 | SEC16A | 2.12.51 |
| NDUFB2 | 2.6 | KIF26B | 2.1 | ATP6AP1 | 2.5.26 | TRIM25 | 2.12.51 |
| UQCR11 | 2.6 | ADAR | 2.12 | EMB | 2.5.26 | PCGF3 | 2.12.51 |
| BCL2L1 | 2.6 | RNF213 | 2.12 | LHFPL2 | 2.5.26 | DNM2 | 2.12.51 |
| TIMM8B | 2.6 | MYO9B | 2.12 | MRPL40 | 2.5.26 | SPAG7 | 2.12.51 |
| YPEL3 | 2.6 | AS2 | 2.12 | PDPN | 2.5.26 | SUGP2 | 2.12.51 |
| SCAP | 2.6 | MPEG1 | 2.12 | CCR1 | 2.5.26 | IFITM1 | 2.12.51 |
| PODN | 2.6 | EIF4G1 | 2.12 | MS4A7 | 2.5.26 | GPS1 | 2.12.51 |
| MRPL51 | 2.6 | RALGDS | 2.12 | HAVCR2 | 2.5.26 | PNPLA6 | 2.12.51 |
| C19orf53 | 2.6 | NMT1 | 2.12 | COMT | 2.5.26 | UNC93B1 | 2.12.51 |
| EIF5A | 2.6 | ITGB2 | 2.12 | Clorf162 | 2.5.26 | ORAI1 | 2.12.51 |
| FCGR3B | 2.6 | IFI6 | 2.12 | SIGLEC1 | 2.5.26 | CTSH | 2.12.51 |
| DCTN6 | 2.6 | ILF3 | 2.12 | RNASE6 | 2.5.26 | E2F4 | 2.12.51 |
| TMEM126B | 2.6 | MX1 | 2.12 | SLC31A1 | 2.5.26 | PLXNA1 | 2.12.51 |
| MSRB1 | 2.6 | RAPGEF1 | 2.12 | RAP2B | 2.5.26 | CSNK1G2 | 2.12.51 |
| NDUFB11 | 2.6 | ETV6 | 2.12 | HEXB | 2.5.26 | TYK2 | 2.12.51 |
| R3HDM4 | 2.6 | RCC2 | 2.12 | MBOAT4 | 2.5.26 | GRK2 | 2.12.51 |
| RBM38 | 2.6 | RASA3 | 2.12 | DYNLT1 | 2.5.26 | TCN2 | 2.12.51 |
| CLTB | 2.6 | ADGRE5 | 2.12 | SGSH | 2.5.26 | LARP1 | 2.12.51 |
| ID3 | 2.6 | SLC9A3R1 | 2.12 | NKX2-1 | 2.5.26 | LGALS9 | 2.12.51 |
| KRCC1 | 2.6 | XAF1 | 2.12 | KLF13 | 2.5.26 | ZNFX1 | 2.12.51 |
| POP7 | 2.6 | SEC16A | 2.12 | ADRA2C | 2.5.26 | PDAP1 | 2.12.51 |
| CDC26 | 2.6 | TRIM25 | 2.12 | ADAP2 | 2.5.26 | DUSIL | 2.12.51 |
| RWDD1 | 2.6 | PCGF3 | 2.12 | MMP19 | 2.5.26 | PPARD | 2.12.51 |
| MORF4L2 | 2.6 | OAS3 | 2.12 | TUBA1B | 2.5.26 | MAPK8IP3 | 2.12.51 |
| COX7A1 | 2.6 | THEMIS2 | 2.12 | FOXI3 | 2.5.26 | SHMT2 | 2.12.51 |
| UXT | 2.6 | DNM2 | 2.12 | GSX2 | 2.5.26 | FRMD8 | 2.12.51 |
| BLOC1S1 | 2.6 | SPAG7 | 2.12 | KPNA7 | 2.5.26 | TBC1D17 | 2.12.51 |
| SELENBP1 | 2.6 | MYO1F | 2.12 | TLR7 | 2.5.26 | HYI | 2.12.51 |
| SF3B5 | 2.6 | SUGP2 | 2.12 | FUCA2 | 2.5.26 | ARAP1 | 2.12.51 |
| B3GNT9 | 2.6 | FMNL1 | 2.12 | OR7G1 | 2.5.26 | CHPF | 2.12.51 |
| RGS10 | 2.6 | IFITM1 | 2.12 | ACOT7 | 2.5.26 | FAM53B | 2.12.51 |
| MRPL20 | 2.6 | GPS1 | 2.12 | RPS11 | 2.6.30 | U2AF2 | 2.12.51 |
| NDUFB3 | 2.6 | PNPLA6 | 2.12 | RPL11 | 2.6.30 | TMEM63B | 2.12.51 |
| UQCRH | 2.6 | UNC93B1 | 2.12 | RPL31 | 2.6.30 | SPATA20 | 2.12.51 |
| MED30 | 2.6 | ORAI1 | 2.12 | RPS25 | 2.6.30 | TMEM185B | 2.12.51 |
| MZT2B | 2.6 | IFIT3 | 2.12 | RPS23 | 2.6.30 | PTPN23 | 2.12.51 |
| CSF3R | 2.6 | CTSH | 2.12 | RPL32 | 2.6.30 | TMEM175 | 2.12.51 |
| RPL29 | 2.6 | E2F4 | 2.12 | RPS20 | 2.6.30 | SCAF1 | 2.12.51 |
| ROMO1 | 2.6 | PLXNA1 | 2.12 | RPS16 | 2.6.30 | RNF126 | 2.12.51 |
| NDUFC2 | 2.6 | NFAM1 | 2.12 | RPS21 | 2.6.30 | APOC1 | 2.12.51 |
| CRYGB | 2.6 | VOPP1 | 2.12 | RACK1 | 2.6.30 | RHOT2 | 2.12.51 |
| GALNT10 | 2.6 | COTL1 | 2.12 | RPL34 | 2.6.30 | TNS3 | 2.12.51 |
| TMA7 | 2.6 | CSNK1G2 | 2.12 | RPL19 | 2.6.30 | APOE | 2.12.51 |
| LAMTOR4 | 2.6 | VASP | 2.12 | RPL38 | 2.6.30 | SDF2L1 | 2.12.51 |
| GPR34 | 2.6 | TYK2 | 2.12 | RPL27 | 2.6.30 | MOGS | 2.12.51 |
| BAIAP3 | 2.6 | PTPN6 | 2.12 | RPL23 | 2.6.30 | TGM2 | 2.12.51 |
| KCNF1 | 2.6 | GRK2 | 2.12 | UBA52 | 2.6.30 | SAMD1 | 2.12.51 |
| ZDHHC1 | 2.6 | TCN2 | 2.12 | TPT1 | 2.6.30 | IER5L | 2.12.51 |
| TMEM256 | 2.6 | LARP1 | 2.12 | RPS6 | 2.6.30 | DNAH1 | 2.12.51 |
| MRPS36 | 2.6 | RUNX3 | 2.12 | RPL35A | 2.6.30 | MRPS2 | 2.12.51 |
| IL2 | 2.6 | ITGAX | 2.12 | RPL4 | 2.6.30 | OR10Q1 | 2.12.51 |
| PHOSPHO1 | 2.6 | NCF1 | 2.12 | RPS8 | 2.6.30 | FSCN1 | 2.12.51 |
| COX4I1 | 2.6 | BST2 | 2.12 | RPL10A | 2.6.30 | INAFM1 | 2.12.51 |
| NAA38 | 2.6 | PREX1 | 2.12 | BTF3 | 2.6.30 | RPS6KA4 | 2.12.51 |
| CRYBB3 | 2.6 | LGALS9 | 2.12 | RPL15 | 2.6.30 | HMG20B | 2.12.51 |
| RAMP3 | 2.6 | ZNFX1 | 2.12 | RPS12 | 2.6.30 | KCNE2 | 2.12.51 |
| IFITM5 | 2.6 | PDAP1 | 2.12 | RPL18 | 2.6.30 | RTN4RL1 | 2.12.51 |
| MRPL21 | 2.6 | MX2 | 2.12 | RPL36 | 2.6.30 | PARP12 | 2.12.51 |
| FGF8 | 2.6 | DUS1L | 2.12 | RPL3 | 2.6.30 | ABHD14A | 2.12.51 |
| CD177 | 2.6 | PPARD | 2.12 | RPS13 | 2.6.30 | CDA | 2.12.51 |
| RCN3 | 2.6 | MAPK8IP3 | 2.12 | RPLP2 | 2.6.30 | GIGYF1 | 2.12.51 |
| OPRD1 | 2.6 | SHMT2 | 2.12 | COX6B1 | 2.6.30 | CRACR2B | 2.12.51 |
| IGFBP1 | 2.6 | EFHD2 | 2.12 | RPL13A | 2.6.30 | KCTD17 | 2.12.51 |
| SRMS | 2.6 | ISG15 | 2.12 | UBB | 2.6.30 | TRAF7 | 2.12.51 |
| NUDT18 | 2.6 | FRMD8 | 2.12 | RPS3 | 2.6.30 | GRP | 2.12.51 |
| DPPA5 | 2.6 | TBC1D17 | 2.12 | RPL37A | 2.6.30 | CRIP1 | 2.12.51 |
| RPS5 | 2.6 | HYI | 2.12 | UQCRQ | 2.6.30 | ADAMTS10 | 2.12.51 |
| RPL12 | 2.6 | OAS1 | 2.12 | RPL24 | 2.6.30 | SPINT3 | 2.12.51 |
| AFF1 | 2.6 | SERPINA1 | 2.12 | NDUFA1 | 2.6.30 | SMR3A | 2.12.51 |
| TOMM7 | 2.6 | ARAP1 | 2.12 | RPL14 | 2.6.30 | SCARF2 | 2.12.51 |
| RPL28 | 2.6 | CHPF | 2.12 | COX7A2 | 2.6.30 | CHKB | 2.12.51 |
| PTPRA | 2.6 | NADK | 2.12 | RPS2 | 2.6.30 | KLHL17 | 2.12.51 |
| RPL26 | 2.6 | FAM53B | 2.12 | RPS24 | 2.6.30 | BEX2 | 2.12.51 |
| GPX4 | 2.6 | U2AF2 | 2.12 | UBL5 | 2.6.30 | DKKL1 | 2.12.51 |
| LIMA1 | 2.6 | CD40 | 2.12 | FAU | 2.6.30 | EVA1B | 2.12.51 |
| KLF10 | 2.6 | TMEM63B | 2.12 | RPLP1 | 2.6.30 | LRRC25 | 2.12.51 |
| TXN | 2.6 | RGS19 | 2.12 | HINT1 | 2.6.30 | OR11H12 | 2.12.51 |
| DCAF12 | 2.6 | ABI3 | 2.12 | DAD1 | 2.6.30 | VASH1 | 2.12.51 |
| RPS14 | 2.6 | SPATA20 | 2.12 | VAMP5 | 2.6.30 | DNMT1 | 2.12.51 |
| CHSY1 | 2.6 | TMEM185B | 2.12 | PFDN5 | 2.6.30 | OR11H1 | 2.12.51 |
| PSMB7 | 2.6 | PTPN23 | 2.12 | POLR2L | 2.6.30 | MTA1 | 2.12.51 |
| RPS15A | 2.6 | RNF166 | 2.12 | RPL36AL | 2.6.30 | CBX6 | 2.12.51 |
| EEF1B2 | 2.6 | ZBTB7B | 2.12 | OST4 | 2.6.30 | SGSM3 | 2.12.51 |
| NDUFA4 | 2.6 | TMEM175 | 2.12 | MYL12B | 2.6.30 | VAC14 | 2.12.51 |
| NDUFAF3 | 2.6 | SCAF1 | 2.12 | RPL35 | 2.6.30 | SETDIA | 2.12.51 |
| NUTF2 | 2.6 | RNF126 | 2.12 | LPAR1 | 2.6.30 | PLEKHA2 | 2.12.51 |
| RPL41 | 2.6 | IL1RN | 2.12 | RPS19 | 2.6.30 | STX10 | 2.12.51 |
| ATP6V1E1 | 2.6 | APOC1 | 2.12 | POLR2K | 2.6.30 | VWA1 | 2.12.51 |
| LXN | 2.6 | RHOT2 | 2.12 | RPS27A | 2.6.30 | IL3RA | 2.12.51 |
| ECE1 | 2.6 | ARHGAP45 | 2.12 | PFDN2 | 2.6.30 | MEN1 | 2.12.51 |
| BRK1 | 2.6 | EPSTI1 | 2.12 | RPS15 | 2.6.30 | AVPI1 | 2.12.51 |
| MFSD14B | 2.6 | TNS3 | 2.12 | CWC15 | 2.6.30 | TOLLIP | 2.12.51 |
| FKBP3 | 2.6 | APOBEC3A | 2.12 | RPL5 | 2.6.30 | TPST2 | 2.12.51 |
| RNF181 | 2.6 | SLC11A1 | 2.12 | SSR4 | 2.6.30 | GIPC1 | 2.12.51 |
| SNRPD2 | 2.6 | APOE | 2.12 | RPL13 | 2.6.30 | GAGE2A | 2.12.51 |
| RPS29 | 2.6 | SDF2L1 | 2.12 | EIF3H | 2.6.30 | ZNF580 | 2.12.51 |
| RPS10 | 2.6 | ARHGAP4 | 2.12 | RPL39 | 2.6.30 | C15orf39 | 2.12.51 |
| EIF2S3 | 2.6 | MOGS | 2.12 | CNBP | 2.6.30 | CLCN7 | 2.12.51 |
| GOLGA7 | 2.6 | CYP2S1 | 2.12 | MT1E | 2.6.30 | ZC3H12A | 2.12.51 |
| DUSP7 | 2.6 | SIGLEC14 | 2.12 | RPL27A | 2.6.30 | MEX3D | 2.12.51 |
| NDUFB4 | 2.6 | TGM2 | 2.12 | VAMP8 | 2.6.30 | THOP1 | 2.12.51 |
| EIF4B | 2.6 | SIRPB2 | 2.12 | RPL30 | 2.6.30 | PRR5 | 2.12.51 |
| SP2 | 2.6 | SAMD1 | 2.12 | EDF1 | 2.6.30 | TMEM204 | 2.12.51 |
| HSPE1 | 2.6 | IER5L | 2.12 | RPS4X | 2.6.30 | OVOL3 | 2.12.51 |
| NDUFA13 | 2.6 | NECTIN2 | 2.12 | COA3 | 2.6.30 | MTX1 | 2.12.51 |
| LAMTOR5 | 2.6 | IMPDH1 | 2.12 | RPL37 | 2.6.30 | PRAMEF1 | 2.12.51 |
| MRPS18A | 2.6 | ANO9 | 2.12 | RPLPO | 2.6.30 | FAM24B | 2.12.51 |
| PPIC | 2.6 | DNAH1 | 2.12 | COX7C | 2.6.30 | ITGB2 | 2.12.53 |
| TADA3 | 2.6 | MRPS2 | 2.12 | RPL7A | 2.6.30 | THEMIS2 | 2.12.53 |
| LBH | 2.6 | ARHGEF1 | 2.12 | TMEM258 | 2.6.30 | MYO1F | 2.12.53 |
| S100A16 | 2.6 | NUDT14 | 2.12 | UQCR10 | 2.6.30 | FMNL1 | 2.12.53 |
| NDUFS6 | 2.6 | OR10Q1 | 2.12 | RPL18A | 2.6.30 | NFAM1 | 2.12.53 |
| SLC35B2 | 2.6 | FSCN1 | 2.12 | TIMM8B | 2.6.30 | VOPP1 | 2.12.53 |
| COX6A1 | 2.6 | CCL8 | 2.12 | C19orf53 | 2.6.30 | COTL1 | 2.12.53 |
| KNCN | 2.6 | TRABD | 2.12 | MSRB1 | 2.6.30 | VASP | 2.12.53 |
| RGS2 | 2.6 | INAFM1 | 2.12 | ID3 | 2.6.30 | PTPN6 | 2.12.53 |
| NR2F6 | 2.6 | RPS6KA4 | 2.12 | KRCC1 | 2.6.30 | RUNX3 | 2.12.53 |
| MAP2K3 | 2.6 | ACHE | 2.12 | POP7 | 2.6.30 | ITGAX | 2.12.53 |
| MT1M | 2.6 | HMG20B | 2.12 | CDC26 | 2.6.30 | NCF1 | 2.12.53 |
| TMEM179B | 2.6 | KCNE2 | 2.12 | RWDD1 | 2.6.30 | PREX1 | 2.12.53 |
| OR4D11 | 2.6 | RTN4RL1 | 2.12 | MORF4L2 | 2.6.30 | EFHD2 | 2.12.53 |
| INSM2 | 2.6 | PARP12 | 2.12 | COX7A1 | 2.6.30 | SERPINA1 | 2.12.53 |
| ZNF358 | 2.6 | ABHD14A | 2.12 | SF3B5 | 2.6.30 | NADK | 2.12.53 |
| SWI5 | 2.6 | PRSS36 | 2.12 | MRPL20 | 2.6.30 | CD40 | 2.12.53 |
| LRRC32 | 2.6 | CDA | 2.12 | NDUFB3 | 2.6.30 | RGS19 | 2.12.53 |
| SLC31A2 | 2.6 | TPRN | 2.12 | UQCRH | 2.6.30 | ABI3 | 2.12.53 |
| VN1R4 | 2.6 | GIGYF1 | 2.12 | MED30 | 2.6.30 | RNF166 | 2.12.53 |
| FNDC4 | 2.6 | CRACR2B | 2.12 | MZT2B | 2.6.30 | ZBTB7B | 2.12.53 |
| MRPL41 | 2.6 | KCTD17 | 2.12 | RPL29 | 2.6.30 | ARHGAP45 | 2.12.53 |
| DCD | 2.6 | TRAF7 | 2.12 | ROMO1 | 2.6.30 | SLC11A1 | 2.12.53 |
| KLHL35 | 2.6 | UNC13D | 2.12 | TMA7 | 2.6.30 | ARHGAP4 | 2.12.53 |
| LAGE3 | 2.6 | GRP | 2.12 | LAMTOR4 | 2.6.30 | SIGLEC14 | 2.12.53 |
| OR2T34 | 2.6 | TSPAN32 | 2.12 | TMEM256 | 2.6.30 | SIRPB2 | 2.12.53 |
| TMEM54 | 2.6 | CRIP1 | 2.12 | COX4I1 | 2.6.30 | NECTIN2 | 2.12.53 |
| ABR | 2.6 | ADAMTS10 | 2.12 | NAA38 | 2.6.30 | IMPDH1 | 2.12.53 |
| TMEM86B | 2.6 | SPINT3 | 2.12 | CRYBB3 | 2.6.30 | ARHGEF1 | 2.12.53 |
| POM121L12 | 2.6 | INO80E | 2.12 | MRPL21 | 2.6.30 | NUDT14 | 2.12.53 |
| LAPTM4A | 2.7 | GZMH | 2.12 | IGFBP1 | 2.6.30 | TRABD | 2.12.53 |
| RHOA | 2.7 | ZC3H3 | 2.12 | SRMS | 2.6.30 | PRSS36 | 2.12.53 |
| PABPC1 | 2.7 | PCSK4 | 2.12 | RPS5 | 2.6.30 | TPRN | 2.12.53 |
| RAB7A | 2.7 | TFAP2E | 2.12 | RPL12 | 2.6.30 | UNC13D | 2.12.53 |
| EIF4G2 | 2.7 | ANO8 | 2.12 | TOMM7 | 2.6.30 | GZMH | 2.12.53 |
| CSDE1 | 2.7 | SMR3A | 2.12 | RPL28 | 2.6.30 | ZC3H3 | 2.12.53 |
| PTTG1IP | 2.7 | IFNA5 | 2.12 | PTPRA | 2.6.30 | TFAP2E | 2.12.53 |
| TPM4 | 2.7 | RAB42 | 2.12 | RPL26 | 2.6.30 | ANO8 | 2.12.53 |
| HNRNPK | 2.7 | PGA5 | 2.12 | GPX4 | 2.6.30 | RAB42 | 2.12.53 |
| SRP14 | 2.7 | ARHGAP39 | 2.12 | KLF10 | 2.6.30 | CCDC86 | 2.12.53 |
| CAPZB | 2.7 | SCARF2 | 2.12 | TXN | 2.6.30 | SDS | 2.12.53 |
| NUCKS1 | 2.7 | SOGA3 | 2.12 | RPS14 | 2.6.30 | CLDN24 | 2.12.53 |
| ANXA2 | 2.7 | CHKB | 2.12 | RPS15A | 2.6.30 | PCYT2 | 2.12.53 |
| HSP90AA1 | 2.7 | KLHL17 | 2.12 | EEF1B2 | 2.6.30 | CYTH1 | 2.12.53 |
| PIGT | 2.7 | BEX2 | 2.12 | NDUFA4 | 2.6.30 | DESI1 | 2.12.53 |
| ANXA1 | 2.7 | CCDC86 | 2.12 | NDUFAF3 | 2.6.30 | FGR | 2.12.53 |
| PTP4A2 | 2.7 | PAQR4 | 2.12 | RPL41 | 2.6.30 | PPP6R1 | 2.12.53 |
| S100A4 | 2.7 | SDS | 2.12 | ATP6V1E1 | 2.6.30 | PIK3R5 | 2.12.53 |
| HSPA8 | 2.7 | CLDN24 | 2.12 | BRK1 | 2.6.30 | RIN3 | 2.12.53 |
| PEA15 | 2.7 | DKKL1 | 2.12 | FKBP3 | 2.6.30 | BCL3 | 2.12.53 |
| CALR | 2.7 | CAMK1 | 2.12 | SNRPD2 | 2.6.30 | ACRBP | 2.12.53 |
| CAPNS1 | 2.7 | EVA1B | 2.12 | RPS29 | 2.6.30 | APOBR | 2.12.53 |
| ZBTB4 | 2.7 | ASB16 | 2.12 | RPS10 | 2.6.30 | TMEM158 | 2.12.53 |
| ARL6IP5 | 2.7 | LRRC25 | 2.12 | EIF2S3 | 2.6.30 | GJA4 | 2.12.53 |
| CD99 | 2.7 | MMP28 | 2.12 | GOLGA7 | 2.6.30 | CRYGD | 2.12.53 |
| IL6ST | 2.7 | PCYT2 | 2.12 | NDUFB4 | 2.6.30 | ISYNA1 | 2.12.53 |
| MYL6 | 2.7 | OR11H12 | 2.12 | EIF4B | 2.6.30 | SIRPB1 | 2.12.53 |
| EPB41L2 | 2.7 | VASH1 | 2.12 | HSPE1 | 2.6.30 | EPHB6 | 2.12.53 |
| S100A10 | 2.7 | DNMT1 | 2.12 | MRPS18A | 2.6.30 | AP3D1 | 2.13.55 |
| APLP2 | 2.7 | CNIH2 | 2.12 | PPIC | 2.6.30 | POLR2A | 2.13.55 |
| VCAN | 2.7 | OR11H1 | 2.12 | LBH | 2.6.30 | BRD4 | 2.13.55 |
| RAC1 | 2.7 | DEGS2 | 2.12 | S100A16 | 2.6.30 | STAT6 | 2.13.55 |
| ARPC2 | 2.7 | CYTH1 | 2.12 | NDUFS6 | 2.6.30 | INPP5D | 2.13.55 |
| NFE2L2 | 2.7 | MTA1 | 2.12 | KNCN | 2.6.30 | UBA7 | 2.13.55 |
| S100A11 | 2.7 | CBX6 | 2.12 | NR2F6 | 2.6.30 | PIEZO1 | 2.13.55 |
| CDV3 | 2.7 | SGSM3 | 2.12 | MT1M | 2.6.30 | ZMIZ2 | 2.13.55 |
| DYNLL1 | 2.7 | DESI1 | 2.12 | ZNF358 | 2.6.30 | KMT2D | 2.13.55 |
| ITM2B | 2.7 | VAC14 | 2.12 | SWI5 | 2.6.30 | FLOT2 | 2.13.55 |
| LUM | 2.7 | SETDIA | 2.12 | SLC31A2 | 2.6.30 | CPNE1 | 2.13.55 |
| GPNMB | 2.7 | FGR | 2.12 | VN1R4 | 2.6.30 | LASP1 | 2.13.55 |
| LDHA | 2.7 | PLEKHA2 | 2.12 | FNDC4 | 2.6.30 | FXYD5 | 2.13.55 |
| MYADM | 2.7 | PPP6R1 | 2.12 | MRPL41 | 2.6.30 | MEF2D | 2.13.55 |
| TGFBI | 2.7 | PIK3R5 | 2.12 | OR2T34 | 2.6.30 | ARIDIA | 2.13.55 |
| SAT1 | 2.7 | RIN3 | 2.12 | POM121L12 | 2.6.30 | WBP2 | 2.13.55 |
| DAZAP2 | 2.7 | STX10 | 2.12 | HEG1 | 2.6.31 | CAPN1 | 2.13.55 |
| SDC2 | 2.7 | VWA1 | 2.12 | NACA | 2.6.31 | ACADVL | 2.13.55 |
| ARF4 | 2.7 | IL3RA | 2.12 | EIF3L | 2.6.31 | PRR14 | 2.13.55 |
| CALM1 | 2.7 | MEN1 | 2.12 | SHISA5 | 2.6.31 | SNRNP70 | 2.13.55 |
| IFI16 | 2.7 | CMPK2 | 2.12 | CD59 | 2.6.31 | NUP62 | 2.13.55 |
| PCOLCE | 2.7 | AVPI1 | 2.12 | CRTAP | 2.6.31 | SIPA1 | 2.13.55 |
| A2M | 2.7 | TOLLIP | 2.12 | SULF2 | 2.6.31 | HNRNPL | 2.13.55 |
| YWHAZ | 2.7 | BCL3 | 2.12 | DYNLRB1 | 2.6.31 | SRRT | 2.13.55 |
| YWHAB | 2.7 | OR6K2 | 2.12 | C12orf57 | 2.6.31 | NOTCH1 | 2.13.55 |
| HIF1A | 2.7 | TPST2 | 2.12 | SUPT4H1 | 2.6.31 | INTS1 | 2.13.55 |
| CLIC4 | 2.7 | GIPC1 | 2.12 | IDH2 | 2.6.31 | ELF4 | 2.13.55 |
| GNB1 | 2.7 | GAGE2A | 2.12 | TMUB2 | 2.6.31 | USF2 | 2.13.55 |
| SPTAN1 | 2.7 | BMF | 2.12 | PHB2 | 2.6.31 | NPIPB3 | 2.13.55 |
| APP | 2.7 | ZNF580 | 2.12 | MAPK3 | 2.6.31 | ARHGEF2 | 2.13.55 |
| PECAM1 | 2.7 | C15orf39 | 2.12 | FKBP10 | 2.6.31 | MAPIS | 2.13.55 |
| SUMO3 | 2.7 | CLCN7 | 2.12 | TOB2 | 2.6.31 | WAS | 2.13.55 |
| GOLIM4 | 2.7 | ACRBP | 2.12 | COX8A | 2.6.31 | ULK1 | 2.13.55 |
| OS9 | 2.7 | ZC3H12A | 2.12 | COX14 | 2.6.31 | TNRC18 | 2.13.55 |
| TGOLN2 | 2.7 | MEX3D | 2.12 | SCAP | 2.6.31 | GTPBP6 | 2.13.55 |
| PLXDC2 | 2.7 | THOP1 | 2.12 | PODN | 2.6.31 | CDK2AP2 | 2.13.55 |
| EFEMP1 | 2.7 | APOBR | 2.12 | MRPL51 | 2.6.31 | RXRA | 2.13.55 |
| HDLBP | 2.7 | PRR5 | 2.12 | EIF5A | 2.6.31 | RNF44 | 2.13.55 |
| PSMC3 | 2.7 | B3GAT3 | 2.12 | DCTN6 | 2.6.31 | RELA | 2.13.55 |
| NONO | 2.7 | TMEM204 | 2.12 | CLTB | 2.6.31 | MXD4 | 2.13.55 |
| UBE2D3 | 2.7 | OVOL3 | 2.12 | BLOC1S1 | 2.6.31 | CYTH4 | 2.13.55 |
| TGFBR2 | 2.7 | MTX1 | 2.12 | SELENBP1 | 2.6.31 | NME3 | 2.13.55 |
| CAVI | 2.7 | TMEM158 | 2.12 | B3GNT9 | 2.6.31 | SASH3 | 2.13.55 |
| SLC38A2 | 2.7 | GJA4 | 2.12 | RGS10 | 2.6.31 | DDX41 | 2.13.55 |
| TPI1 | 2.7 | CRYGD | 2.12 | GALNT10 | 2.6.31 | IRF9 | 2.13.55 |
| XRCC6 | 2.7 | ISYNA1 | 2.12 | ZDHHC1 | 2.6.31 | ADAM8 | 2.13.55 |
| MAT2A | 2.7 | SMIM1 | 2.12 | IL2 | 2.6.31 | DCAF15 | 2.13.55 |
| MARCKS | 2.7 | PRAMEF1 | 2.12 | RCN3 | 2.6.31 | PCNX3 | 2.13.55 |
| ATP1A1 | 2.7 | FAM24B | 2.12 | AFF1 | 2.6.31 | ATP13A1 | 2.13.55 |
| ECM2 | 2.7 | SIRPB1 | 2.12 | LIMA1 | 2.6.31 | CRTC2 | 2.13.55 |
| USP22 | 2.7 | EPHB6 | 2.12 | CHSY1 | 2.6.31 | JUP | 2.13.55 |
| FKBP1A | 2.7 | GOLGA6L10 | 2.12 | NUTF2 | 2.6.31 | DHCR24 | 2.13.55 |
| EXOSC10 | 2.7 | AP3D1 | 2.13 | ECE1 | 2.6.31 | TMEM104 | 2.13.55 |
| NFKBIA | 2.7 | POLR2A | 2.13 | DUSP7 | 2.6.31 | EHD1 | 2.13.55 |
| CAP1 | 2.7 | BRD4 | 2.13 | SP2 | 2.6.31 | GMIP | 2.13.55 |
| SURF4 | 2.7 | STAT6 | 2.13 | TADA3 | 2.6.31 | FBRSL1 | 2.13.55 |
| DSTN | 2.7 | INPP5D | 2.13 | SLC35B2 | 2.6.31 | IER2 | 2.13.55 |
| HNRNPUL1 | 2.7 | UBA7 | 2.13 | COX6A1 | 2.6.31 | REEP4 | 2.13.55 |
| WDR1 | 2.7 | SYNPO | 2.13 | INSM2 | 2.6.31 | SREBF1 | 2.13.55 |
| RBM39 | 2.7 | PIEZO1 | 2.13 | LRRC32 | 2.6.31 | HELZ2 | 2.13.55 |
| AP2M1 | 2.7 | ZMIZ2 | 2.13 | LAGE3 | 2.6.31 | KIAA2013 | 2.13.55 |
| SF3B1 | 2.7 | MED15 | 2.13 | TMEM54 | 2.6.31 | IRF5 | 2.13.55 |
| TRPS1 | 2.7 | KMT2D | 2.13 | ABR | 2.6.31 | SPPL2B | 2.13.55 |
| DAB2 | 2.7 | FLOT2 | 2.13 | ATP6VOE1 | 2.6.32 | GYS1 | 2.13.55 |
| JMJDIC | 2.7 | CPNE1 | 2.13 | NDUFS5 | 2.6.32 | TMEM115 | 2.13.55 |
| IFITM3 | 2.7 | SLC29A1 | 2.13 | PRDX1 | 2.6.32 | TPCN1 | 2.13.55 |
| UBA1 | 2.7 | CIITA | 2.13 | NDUFA12 | 2.6.32 | IL4R | 2.13.55 |
| YBX1 | 2.7 | LASP1 | 2.13 | EIF3K | 2.6.32 | NFKBIE | 2.13.55 |
| NFAT5 | 2.7 | FXYD5 | 2.13 | TMEM50A | 2.6.32 | ABTB1 | 2.13.55 |
| CHMP4B | 2.7 | MEF2D | 2.13 | NDUFB1 | 2.6.32 | MCOLN1 | 2.13.55 |
| LGMN | 2.7 | ARIDIA | 2.13 | SLIRP | 2.6.32 | KLF16 | 2.13.55 |
| SNX3 | 2.7 | SLC6A6 | 2.13 | SEC61B | 2.6.32 | TFE3 | 2.13.55 |
| CD55 | 2.7 | AKNA | 2.13 | COX7B | 2.6.32 | DTX2 | 2.13.55 |
| COL5A2 | 2.7 | WBP2 | 2.13 | FAM32A | 2.6.32 | AGRN | 2.13.55 |
| OXA1L | 2.7 | CAPN1 | 2.13 | COX6C | 2.6.32 | FHL3 | 2.13.55 |
| LMAN2 | 2.7 | RHBDF2 | 2.13 | VPS29 | 2.6.32 | PIGQ | 2.13.55 |
| ZMIZ1 | 2.7 | ACADVL | 2.13 | NDUFB2 | 2.6.32 | NAALADL1 | 2.13.55 |
| HCFC1 | 2.7 | ACTN1 | 2.13 | UQCR11 | 2.6.32 | LCAT | 2.13.55 |
| GTF3A | 2.7 | PRR14 | 2.13 | TMEM126B | 2.6.32 | RHBDD2 | 2.13.55 |
| TMED9 | 2.7 | SNRNP70 | 2.13 | NDUFB11 | 2.6.32 | TREM2 | 2.13.55 |
| TMEM259 | 2.7 | NUP62 | 2.13 | UXT | 2.6.32 | RABGGTA | 2.13.55 |
| STAU1 | 2.7 | SIPA1 | 2.13 | NDUFC2 | 2.6.32 | PET100 | 2.13.55 |
| YWHAH | 2.7 | HNRNPL | 2.13 | CRYGB | 2.6.32 | EMILIN1 | 2.13.55 |
| GRINA | 2.7 | SRRT | 2.13 | GPR34 | 2.6.32 | KCNK7 | 2.13.55 |
| ESD | 2.7 | CSRP1 | 2.13 | MRPS36 | 2.6.32 | ARSA | 2.13.55 |
| PSMA7 | 2.7 | DLGAP4 | 2.13 | CD177 | 2.6.32 | SLC12A7 | 2.13.55 |
| STOM | 2.7 | NOTCH1 | 2.13 | PSMB7 | 2.6.32 | CTNS | 2.13.55 |
| CDC42 | 2.7 | MYO1G | 2.13 | LXN | 2.6.32 | NEUROG2 | 2.13.55 |
| WBP1L | 2.7 | SLC17A9 | 2.13 | MFSD14B | 2.6.32 | C8orf82 | 2.13.55 |
| WASF2 | 2.7 | INTS1 | 2.13 | RNF181 | 2.6.32 | ANKRD23 | 2.13.55 |
| SBDS | 2.7 | ELF4 | 2.13 | NDUFA13 | 2.6.32 | ZNF497 | 2.13.55 |
| HSPB1 | 2.7 | USF2 | 2.13 | LAMTOR5 | 2.6.32 | ZNF628 | 2.13.55 |
| CITED2 | 2.7 | CORO1A | 2.13 | TMEM179B | 2.6.32 | IFNA14 | 2.13.55 |
| NSA2 | 2.7 | NR1H3 | 2.13 | OR4D11 | 2.6.32 | TRIM60 | 2.13.55 |
| DAP | 2.7 | NPIPB3 | 2.13 | DCD | 2.6.32 | FUT7 | 2.13.55 |
| P4HB | 2.7 | PTK2B | 2.13 | LAPTM4A | 2.7.35 | NBPF6 | 2.13.55 |
| CXCL12 | 2.7 | ARHGEF2 | 2.13 | RHOA | 2.7.35 | UGT2B28 | 2.13.55 |
| ENG | 2.7 | ARHGAP9 | 2.13 | PABPC1 | 2.7.35 | XRCC3 | 2.13.55 |
| NOP10 | 2.7 | CXCL9 | 2.13 | RAB7A | 2.7.35 | PRRC2B | 2.13.55 |
| PRNP | 2.7 | MAPIS | 2.13 | EIF4G2 | 2.7.35 | STK40 | 2.13.55 |
| IRAK1 | 2.7 | IL4I1 | 2.13 | CSDE1 | 2.7.35 | NXF1 | 2.13.55 |
| TUBB4B | 2.7 | WAS | 2.13 | TPM4 | 2.7.35 | TOM1 | 2.13.55 |
| ECM1 | 2.7 | ULK1 | 2.13 | HNRNPK | 2.7.35 | CCDC69 | 2.13.55 |
| H6PD | 2.7 | TNRC18 | 2.13 | SRP14 | 2.7.35 | CMIP | 2.13.55 |
| MDH2 | 2.7 | SLC9A1 | 2.13 | CAPZB | 2.7.35 | RBCK1 | 2.13.55 |
| FGL2 | 2.7 | GTPBP6 | 2.13 | NUCKS1 | 2.7.35 | SRF | 2.13.55 |
| CSF1 | 2.7 | B3GNT7 | 2.13 | HSP90AA1 | 2.7.35 | CDC25B | 2.13.55 |
| YIPF3 | 2.7 | CDK2AP2 | 2.13 | ANXA1 | 2.7.35 | TOMM34 | 2.13.55 |
| DDX6 | 2.7 | TESK1 | 2.13 | PTP4A2 | 2.7.35 | BCL9L | 2.13.55 |
| XRCC5 | 2.7 | RXRA | 2.13 | S100A4 | 2.7.35 | OTUB1 | 2.13.55 |
| ITGB1 | 2.7 | RNF44 | 2.13 | HSPA8 | 2.7.35 | OR6C70 | 2.13.55 |
| MKNK2 | 2.7 | POU2F2 | 2.13 | CALR | 2.7.35 | ABHD12 | 2.13.55 |
| PSMD8 | 2.7 | PILRA | 2.13 | CAPNS1 | 2.7.35 | PNKD | 2.13.55 |
| TPD52L2 | 2.7 | PIK3CD | 2.13 | ARL6IP5 | 2.7.35 | KAT2A | 2.13.55 |
| MYL12A | 2.7 | SBF1 | 2.13 | CD99 | 2.7.35 | ERAL1 | 2.13.55 |
| VEGFB | 2.7 | SLAMF8 | 2.13 | IL6ST | 2.7.35 | HPCAL1 | 2.13.55 |
| LMF2 | 2.7 | RELA | 2.13 | MYL6 | 2.7.35 | ATG16L2 | 2.13.55 |
| MAN1A1 | 2.7 | ARHGEF10L | 2.13 | EPB41L2 | 2.7.35 | SEMA4C | 2.13.55 |
| MYL9 | 2.7 | MXD4 | 2.13 | S100A10 | 2.7.35 | NFKBID | 2.13.55 |
| SAMHD1 | 2.7 | CYTH4 | 2.13 | VCAN | 2.7.35 | ALDH3B1 | 2.13.55 |
| DST | 2.7 | PLA2G2D | 2.13 | RAC1 | 2.7.35 | CAPZA3 | 2.13.55 |
| PEBP1 | 2.7 | SNX20 | 2.13 | ARPC2 | 2.7.35 | CCDC124 | 2.13.55 |
| RABAC1 | 2.7 | GRK6 | 2.13 | NFE2L2 | 2.7.35 | DEFB134 | 2.13.55 |
| EPAS1 | 2.7 | JAK3 | 2.13 | S100A11 | 2.7.35 | UGT1A6 | 2.13.55 |
| RAN | 2.7 | SLC2A5 | 2.13 | CDV3 | 2.7.35 | PILRB | 2.13.55 |
| GPR108 | 2.7 | PLCB2 | 2.13 | DYNLL1 | 2.7.35 | BOP1 | 2.13.55 |
| SET | 2.7 | ABCA2 | 2.13 | ITM2B | 2.7.35 | PARD6A | 2.13.55 |
| BTG1 | 2.7 | SCYL1 | 2.13 | LUM | 2.7.35 | UGT2A2 | 2.13.55 |
| RTF1 | 2.7 | APOBEC3G | 2.13 | MYADM | 2.7.35 | KRTAP5-9 | 2.13.55 |
| JAK1 | 2.7 | NME3 | 2.13 | SAT1 | 2.7.35 | CIITA | 2.13.57 |
| NUCB1 | 2.7 | CHERP | 2.13 | DAZAP2 | 2.7.35 | AKNA | 2.13.57 |
| C6orf62 | 2.7 | IKZF3 | 2.13 | SDC2 | 2.7.35 | RHBDF2 | 2.13.57 |
| PTBP1 | 2.7 | PTOV1 | 2.13 | ARF4 | 2.7.35 | MYO1G | 2.13.57 |
| CCND1 | 2.7 | SASH3 | 2.13 | CALM1 | 2.7.35 | SLC17A9 | 2.13.57 |
| TPPP3 | 2.7 | DDX41 | 2.13 | IFI16 | 2.7.35 | CORO1A | 2.13.57 |
| VKORC1 | 2.7 | LIMD2 | 2.13 | A2M | 2.7.35 | NR1H3 | 2.13.57 |
| IQSEC1 | 2.7 | EEFSEC | 2.13 | YWHAZ | 2.7.35 | PTK2B | 2.13.57 |
| FOXC1 | 2.7 | IRF9 | 2.13 | YWHAB | 2.7.35 | ARHGAP9 | 2.13.57 |
| ALKBH5 | 2.7 | ADAM8 | 2.13 | HIF1A | 2.7.35 | CXCL9 | 2.13.57 |
| RAB5C | 2.7 | DCAF15 | 2.13 | CLIC4 | 2.7.35 | IL4I1 | 2.13.57 |
| OLFML2B | 2.7 | PTAFR | 2.13 | PECAM1 | 2.7.35 | POU2F2 | 2.13.57 |
| GM2A | 2.7 | PCNX3 | 2.13 | GOLIM4 | 2.7.35 | PILRA | 2.13.57 |
| CLPTM1L | 2.7 | ATP13A1 | 2.13 | TGOLN2 | 2.7.35 | PIK3CD | 2.13.57 |
| GIMAP4 | 2.7 | SEMA4A | 2.13 | PLXDC2 | 2.7.35 | SLAMF8 | 2.13.57 |
| NDST1 | 2.7 | CRTC2 | 2.13 | NONO | 2.7.35 | PLA2G2D | 2.13.57 |
| TMEM109 | 2.7 | ZER1 | 2.13 | UBE2D3 | 2.7.35 | SNX20 | 2.13.57 |
| SMARCC2 | 2.7 | RUSC2 | 2.13 | TGFBR2 | 2.7.35 | GRK6 | 2.13.57 |
| S100A6 | 2.7 | PDLIM7 | 2.13 | CAVI | 2.7.35 | JAK3 | 2.13.57 |
| GNAS | 2.7 | GMPPB | 2.13 | SLC38A2 | 2.7.35 | SLC2A5 | 2.13.57 |
| VMP1 | 2.7 | JUP | 2.13 | TPI1 | 2.7.35 | PLCB2 | 2.13.57 |
| LAMP2 | 2.7 | FGFRL1 | 2.13 | XRCC6 | 2.7.35 | APOBEC3G | 2.13.57 |
| ELOVL1 | 2.7 | DHCR24 | 2.13 | MAT2A | 2.7.35 | IKZF3 | 2.13.57 |
| GLG1 | 2.7 | PITPNM1 | 2.13 | ATP1A1 | 2.7.35 | LIMD2 | 2.13.57 |
| TM9SF2 | 2.7 | HMGA1 | 2.13 | ECM2 | 2.7.35 | PTAFR | 2.13.57 |
| HSP90AB1 | 2.7 | MARCKSL1 | 2.13 | FKBP1A | 2.7.35 | SEMA4A | 2.13.57 |
| CSNK1E | 2.7 | BCKDK | 2.13 | EXOSC10 | 2.7.35 | PITPNM1 | 2.13.57 |
| OTUD5 | 2.7 | LIMS2 | 2.13 | NFKBIA | 2.7.35 | HMGA1 | 2.13.57 |
| TNC | 2.7 | TMEM104 | 2.13 | CAP1 | 2.7.35 | MARCKSL1 | 2.13.57 |
| ILF2 | 2.7 | ACAP1 | 2.13 | DSTN | 2.7.35 | ACAP1 | 2.13.57 |
| MFAP4 | 2.7 | EHD1 | 2.13 | HNRNPUL1 | 2.7.35 | NBEAL2 | 2.13.57 |
| PSMC5 | 2.7 | GMIP | 2.13 | SF3B1 | 2.7.35 | SLC29A3 | 2.13.57 |
| YWHAG | 2.7 | FBRSL1 | 2.13 | TRPS1 | 2.7.35 | ARHGAP30 | 2.13.57 |
| UBE2B | 2.7 | NBEAL2 | 2.13 | DAB2 | 2.7.35 | SLC7A5 | 2.13.57 |
| LDB1 | 2.7 | SLC29A3 | 2.13 | JMJDIC | 2.7.35 | KCNAB2 | 2.13.57 |
| PDIA3 | 2.7 | IER2 | 2.13 | IFITM3 | 2.7.35 | SIGLEC10 | 2.13.57 |
| DGUOK | 2.7 | DNASE1L1 | 2.13 | YBX1 | 2.7.35 | NPS | 2.13.57 |
| RAD23A | 2.7 | REEP4 | 2.13 | NFAT5 | 2.7.35 | LBP | 2.13.57 |
| BNIP3L | 2.7 | SREBF1 | 2.13 | LGMN | 2.7.35 | EDIL3 | 2.13.57 |
| ACTRIA | 2.7 | HELZ2 | 2.13 | SNX3 | 2.7.35 | MUC3A | 2.13.57 |
| NINJ1 | 2.7 | KIAA2013 | 2.13 | CD55 | 2.7.35 | NAT14 | 2.13.57 |
| EPHX1 | 2.7 | IRF5 | 2.13 | COL5A2 | 2.7.35 | SLC25A22 | 2.13.57 |
| FURIN | 2.7 | SPPL2B | 2.13 | OXA1L | 2.7.35 | TUBA4A | 2.13.57 |
| CHD4 | 2.7 | ARHGAP30 | 2.13 | LMAN2 | 2.7.35 | SLA2 | 2.13.57 |
| CD248 | 2.7 | GYS1 | 2.13 | GTF3A | 2.7.35 | RNF113A | 2.13.57 |
| SERINC3 | 2.7 | TMEM115 | 2.13 | STAU1 | 2.7.35 | PRF1 | 2.13.57 |
| ERLEC1 | 2.7 | TPCN1 | 2.13 | GRINA | 2.7.35 | GADD45G | 2.13.57 |
| ITGB5 | 2.7 | SLC7A5 | 2.13 | ESD | 2.7.35 | ALYREF | 2.13.57 |
| CREB5 | 2.7 | KCNAB2 | 2.13 | PSMA7 | 2.7.35 | SPAG4 | 2.13.57 |
| MOBIA | 2.7 | IL4R | 2.13 | STOM | 2.7.35 | SMCO4 | 2.13.57 |
| GTF2F1 | 2.7 | NFKBIE | 2.13 | CDC42 | 2.7.35 | CRELD2 | 2.13.57 |
| BHLHE40 | 2.7 | SIGLEC10 | 2.13 | SBDS | 2.7.35 | DENND6B | 2.13.57 |
| PLVAP | 2.7 | ABTB1 | 2.13 | HSPB1 | 2.7.35 | CD300E | 2.13.57 |
| HNRNPH1 | 2.7 | GIT1 | 2.13 | CITED2 | 2.7.35 | OR2A7 | 2.13.57 |
| ARHGAP1 | 2.7 | RBPMS2 | 2.13 | NSA2 | 2.7.35 | LPAR2 | 2.13.57 |
| FSTL1 | 2.7 | APEH | 2.13 | P4HB | 2.7.35 | IGFLR1 | 2.13.57 |
| CEBPB | 2.7 | MCOLN1 | 2.13 | CXCL12 | 2.7.35 | RTEL1 | 2.13.57 |
| CLPTM1 | 2.7 | NPS | 2.13 | ENG | 2.7.35 | PARVG | 2.13.57 |
| HDGF | 2.7 | KLF16 | 2.13 | NOP10 | 2.7.35 | SLC15A3 | 2.13.57 |
| EIF2AK1 | 2.7 | FSTL3 | 2.13 | MDH2 | 2.7.35 | MAST3 | 2.13.57 |
| FOSL2 | 2.7 | LBP | 2.13 | FGL2 | 2.7.35 | GALNS | 2.13.57 |
| TNFSF10 | 2.7 | TFE3 | 2.13 | DDX6 | 2.7.35 | SULTIE1 | 2.13.57 |
| C1RL | 2.7 | ZNF524 | 2.13 | XRCC5 | 2.7.35 | CD82 | 2.13.57 |
| PPT1 | 2.7 | EDIL3 | 2.13 | ITGB1 | 2.7.35 | LGALS2 | 2.13.57 |
| MAF1 | 2.7 | PPL | 2.13 | MKNK2 | 2.7.35 | ACE | 2.13.57 |
| TAB2 | 2.7 | MUC3A | 2.13 | MYL12A | 2.7.35 | NUAK2 | 2.13.57 |
| ARFGAP3 | 2.7 | DTX2 | 2.13 | MAN1A1 | 2.7.35 | CXCL2 | 2.13.57 |
| CALU | 2.7 | UBALD1 | 2.13 | MYL9 | 2.7.35 | METRN | 2.13.57 |
| RAB31 | 2.7 | MYL5 | 2.13 | SAMHD1 | 2.7.35 | RAB20 | 2.13.57 |
| INPPL 1 | 2.7 | AGRN | 2.13 | DST | 2.7.35 | C19orf38 | 2.13.57 |
| CYP4F8 | 2.7 | FHL3 | 2.13 | PEBP1 | 2.7.35 | PNPO | 2.13.57 |
| CTDSP2 | 2.7 | RECQL4 | 2.13 | EPAS1 | 2.7.35 | DCXR | 2.13.57 |
| ATP1B3 | 2.7 | PIGQ | 2.13 | RAN | 2.7.35 | P2RY11 | 2.13.57 |
| MAGED2 | 2.7 | NAT14 | 2.13 | SET | 2.7.35 | MPC1L | 2.13.57 |
| TAGLN2 | 2.7 | NAALADL1 | 2.13 | RTF1 | 2.7.35 | BIK | 2.13.57 |
| CPSF1 | 2.7 | LCAT | 2.13 | JAK1 | 2.7.35 | DEFA6 | 2.13.57 |
| SLC34A1 | 2.7 | RHBDD2 | 2.13 | C6orf62 | 2.7.35 | USP9Y | 3.17.65 |
| ID1 | 2.7 | STX1A | 2.13 | PTBP1 | 2.7.35 | UTY | 3.17.65 |
| KDELR1 | 2.7 | PLK3 | 2.13 | TPPP3 | 2.7.35 | DDX3Y | 3.17.65 |
| UBXN1 | 2.7 | TREM2 | 2.13 | FOXC1 | 2.7.35 | KDM5D | 3.17.65 |
| C3 | 2.7 | SLC25A22 | 2.13 | RAB5C | 2.7.35 | RPS4Y1 | 3.17.65 |
| ALDH2 | 2.7 | RABGGTA | 2.13 | OLFML2B | 2.7.35 | NLGN4Y | 3.17.65 |
| SNRPD3 | 2.7 | TUBA4A | 2.13 | CLPTMIL | 2.7.35 | PTCHD1 | 3.17.65 |
| TMBIM1 | 2.7 | PET100 | 2.13 | S100A6 | 2.7.35 | IGSF1 | 3.17.65 |
| NRBP2 | 2.7 | SLA2 | 2.13 | GNAS | 2.7.35 | AFF2 | 3.17.65 |
| PLD3 | 2.7 | RNF113A | 2.13 | LAMP2 | 2.7.35 | GUCY2F | 3.17.65 |
| APMAP | 2.7 | CNFN | 2.13 | ELOVL1 | 2.7.35 | DCX | 3.17.65 |
| S1PR1 | 2.7 | PRF1 | 2.13 | TM9SF2 | 2.7.35 | CREG1 | 3.17.65 |
| USB1 | 2.7 | KLRC2 | 2.13 | HSP90AB1 | 2.7.35 | ACE2 | 3.17.65 |
| DYNC1I2 | 2.7 | EMILIN1 | 2.13 | ILF2 | 2.7.35 | ADGRG4 | 3.17.65 |
| EIF6 | 2.7 | SCAND1 | 2.13 | MFAP4 | 2.7.35 | DGKK | 3.17.65 |
| ITM2C | 2.7 | KCNK7 | 2.13 | YWHAG | 2.7.35 | F9 | 3.17.65 |
| CAB39 | 2.7 | GADD45G | 2.13 | UBE2B | 2.7.35 | BRS3 | 3.17.65 |
| SFRP1 | 2.7 | ASIC3 | 2.13 | DGUOK | 2.7.35 | CAPN6 | 3.17.65 |
| CRAT | 2.7 | ZDHHC11B | 2.13 | BNIP3L | 2.7.35 | DACH2 | 3.17.65 |
| COL4A2 | 2.7 | ARSA | 2.13 | CD248 | 2.7.35 | USP26 | 3.17.65 |
| HMGN2 | 2.7 | ALYREF | 2.13 | ERLEC1 | 2.7.35 | PHEX | 3.17.65 |
| VDAC3 | 2.7 | GJC2 | 2.13 | CREB5 | 2.7.35 | FRMPD4 | 3.17.65 |
| LRP10 | 2.7 | SPAG4 | 2.13 | MOBIA | 2.7.35 | ARSF | 3.17.65 |
| ACKR1 | 2.7 | SLC12A7 | 2.13 | PLVAP | 2.7.35 | ZC3H12B | 3.17.65 |
| TCEAL8 | 2.7 | TRIM49C | 2.13 | HNRNPH1 | 2.7.35 | TKTL1 | 3.17.65 |
| OCIAD1 | 2.7 | SMCO4 | 2.13 | FSTL1 | 2.7.35 | ZFY | 3.17.65 |
| TCEAL9 | 2.7 | RFNG | 2.13 | CEBPB | 2.7.35 | MAP7D2 | 3.17.65 |
| UBALD2 | 2.7 | CTNS | 2.13 | HDGF | 2.7.35 | TMEM255A | 3.17.65 |
| BTG2 | 2.7 | CRELD2 | 2.13 | EIF2AK1 | 2.7.35 | AR | 3.17.65 |
| JCHAIN | 2.7 | SPNS1 | 2.13 | PPT1 | 2.7.35 | DDX53 | 3.17.65 |
| SEPHS2 | 2.7 | CSN1S1 | 2.13 | TAB2 | 2.7.35 | PAK3 | 3.17.65 |
| CTDSP1 | 2.7 | ZNF593 | 2.13 | ARFGAP3 | 2.7.35 | FOXR2 | 3.17.65 |
| OGN | 2.7 | MOB2 | 2.13 | RAB31 | 2.7.35 | SSX3 | 3.17.65 |
| CDK16 | 2.7 | NEUROG2 | 2.13 | CTDSP2 | 2.7.35 | NOX1 | 3.17.65 |
| ZBTB47 | 2.7 | DENND6B | 2.13 | ATP1B3 | 2.7.35 | ATP1B4 | 3.17.65 |
| TNFAIP1 | 2.7 | FAM110D | 2.13 | TAGLN2 | 2.7.35 | GPR119 | 3.17.65 |
| PTMS | 2.7 | C8orf82 | 2.13 | ID1 | 2.7.35 | HEPH | 3.17.65 |
| DVL3 | 2.7 | ACADS | 2.13 | C3 | 2.7.35 | DCAF8L2 | 3.17.65 |
| ANKH | 2.7 | TIMM10 | 2.13 | ALDH2 | 2.7.35 | EGFL6 | 3.17.65 |
| CYC1 | 2.7 | ANKRD23 | 2.13 | SNRPD3 | 2.7.35 | TAF7L | 3.17.65 |
| PLPP1 | 2.7 | SMTN | 2.13 | APMAP | 2.7.35 | FAM156A | 3.17.65 |
| ANGPTL1 | 2.7 | ZNF497 | 2.13 | S1PR1 | 2.7.35 | SAGE1 | 3.17.65 |
| SKIL | 2.7 | ZNF628 | 2.13 | USB1 | 2.7.35 | FRMD7 | 3.17.65 |
| CIZ1 | 2.7 | AMTN | 2.13 | DYNC1I2 | 2.7.35 | EIF1AY | 3.17.65 |
| NPIPB5 | 2.7 | RTN4R | 2.13 | EIF6 | 2.7.35 | TMEM35A | 3.17.65 |
| SNRPB2 | 2.7 | NDUFA7 | 2.13 | CAB39 | 2.7.35 | MAGEB3 | 3.17.65 |
| SLC35F6 | 2.7 | IFNA14 | 2.13 | CRAT | 2.7.35 | GPC3 | 3.17.65 |
| AKAP17A | 2.7 | KRTAP22-1 | 2.13 | HMGN2 | 2.7.35 | PASD1 | 3.17.65 |
| ERRFI1 | 2.7 | CD300E | 2.13 | VDAC3 | 2.7.35 | GPR143 | 3.17.65 |
| CALD1 | 2.7 | TRIM60 | 2.13 | ACKR1 | 2.7.35 | RBX1 | 3.17.65 |
| SRSF2 | 2.7 | FUT7 | 2.13 | TCEAL8 | 2.7.35 | PLP1 | 3.17.65 |
| TRMT112 | 2.7 | FOXO6 | 2.13 | OCIAD1 | 2.7.35 | NLRP2B | 3.17.65 |
| BCAP31 | 2.7 | OR2A7 | 2.13 | TCEAL9 | 2.7.35 | OTC | 3.17.65 |
| DAPK3 | 2.7 | LTB4R2 | 2.13 | UBALD2 | 2.7.35 | AKAP14 | 3.17.65 |
| UBAP1 | 2.7 | NBPF6 | 2.13 | BTG2 | 2.7.35 | NCBP2L | 3.17.65 |
| CENPB | 2.7 | LPAR2 | 2.13 | JCHAIN | 2.7.35 | PABPC1L2B | 3.17.65 |
| CDON | 2.7 | UGT2B28 | 2.13 | SEPHS2 | 2.7.35 | KLHL34 | 3.17.65 |
| RGS5 | 2.7 | IGFLR1 | 2.13 | CTDSP1 | 2.7.35 | PLAC1 | 3.17.65 |
| MLLT1 | 2.7 | XRCC3 | 2.13 | OGN | 2.7.35 | SLITRK2 | 3.17.65 |
| UBE2L3 | 2.7 | PHKG1 | 2.13 | PTMS | 2.7.35 | SLC9A3R2 | 3.17.65 |
| CD276 | 2.7 | RTEL1 | 2.13 | DVL3 | 2.7.35 | PCDH11Y | 3.17.65 |
| RPL10 | 2.7 | PHC2 | 2.13 | PLPP1 | 2.7.35 | TEX13B | 3.17.65 |
| ZDHHC22 | 2.7 | PRRC2B | 2.13 | ANGPTL1 | 2.7.35 | FMRINB | 3.17.65 |
| MXRA5 | 2.7 | PARVG | 2.13 | SKIL | 2.7.35 | MAGEB1 | 3.17.65 |
| AP2A2 | 2.7 | STK40 | 2.13 | NPIPB5 | 2.7.35 | TCEAL2 | 3.17.65 |
| TP53I11 | 2.7 | NXF1 | 2.13 | SNRPB2 | 2.7.35 | VGLL1 | 3.17.65 |
| ARPC3 | 2.7 | TOM1 | 2.13 | AKAP17A | 2.7.35 | PNMA5 | 3.17.65 |
| NPTN | 2.7 | CCDC69 | 2.13 | CALD1 | 2.7.35 | SYTL5 | 3.17.65 |
| VMA21 | 2.7 | CMIP | 2.13 | SRSF2 | 2.7.35 | GH2 | 3.17.65 |
| NDUFB10 | 2.7 | RBCK1 | 2.13 | TRMT112 | 2.7.35 | GTSF1L | 3.17.65 |
| ARPCIA | 2.7 | SRF | 2.13 | BCAP31 | 2.7.35 | PAGE2 | 3.17.65 |
| C11orf68 | 2.7 | CDC25B | 2.13 | CENPB | 2.7.35 | TP53TG3D | 3.17.65 |
| GDNF | 2.7 | TOMM34 | 2.13 | CDON | 2.7.35 | MAGEA12 | 3.17.65 |
| PBXIP1 | 2.7 | BCL9L | 2.13 | RGS5 | 2.7.35 | C5orf49 | 3.17.65 |
| IRF2BPL | 2.7 | OTUB1 | 2.13 | MLLT1 | 2.7.35 | VMO1 | 3.17.65 |
| GAS6 | 2.7 | SLC15A3 | 2.13 | AP2A2 | 2.7.35 | IL4 | 3.17.65 |
| PLIN1 | 2.7 | MAST3 | 2.13 | VMA21 | 2.7.35 | PAGE2B | 3.17.65 |
| LYSMD2 | 2.7 | OR6C70 | 2.13 | ARPC1A | 2.7.35 | KRTAP11-1 | 3.17.65 |
| TUBG1 | 2.7 | ABHD12 | 2.13 | C11orf68 | 2.7.35 | MAGEB18 | 3.17.65 |
| PRAF2 | 2.7 | SGTA | 2.13 | PBXIP1 | 2.7.35 | CAMP | 3.17.65 |
| GLTP | 2.7 | PNKD | 2.13 | IRF2BPL | 2.7.35 | PPEF1 | 3.17.65 |
| VDAC1 | 2.7 | PDPK1 | 2.13 | GAS6 | 2.7.35 | TRPC5 | 3.17.65 |
| ZNF418 | 2.7 | GALNS | 2.13 | LYSMD2 | 2.7.35 | VSIG1 | 3.17.65 |
| ARMCX6 | 2.7 | CDKNIA | 2.13 | PRAF2 | 2.7.35 | HS6ST2 | 3.17.65 |
| ID2 | 2.7 | SULT1E1 | 2.13 | VDAC1 | 2.7.35 | SRY | 3.17.65 |
| ATP6AP2 | 2.7 | KAT2A | 2.13 | ZNF418 | 2.7.35 | IL1RAPL1 | 3.17.65 |
| COPS7A | 2.7 | ERAL1 | 2.13 | ARMCX6 | 2.7.35 | BEND2 | 3.17.65 |
| PPP1R14B | 2.7 | HPCAL1 | 2.13 | ATP6AP2 | 2.7.35 | HTR2C | 3.17.65 |
| CLIC5 | 2.7 | ATG16L2 | 2.13 | PPP1R14B | 2.7.35 | GAGE1 | 3.17.65 |
| LY96 | 2.7 | SEMA4C | 2.13 | LY96 | 2.7.35 | ARSH | 3.17.65 |
| MCAM | 2.7 | IQSEC2 | 2.13 | MCAM | 2.7.35 | OR51T1 | 3.17.65 |
| LRRFIP1 | 2.7 | NFKBID | 2.13 | LRRFIP1 | 2.7.35 | MTIF3 | 3.17.65 |
| ECSCR | 2.7 | ALDH3B1 | 2.13 | ECSCR | 2.7.35 | CHRDL1 | 3.17.65 |
| SHANK1 | 2.7 | CD82 | 2.13 | COX5A | 2.7.35 | MAGEB4 | 3.17.65 |
| MAP2K2 | 2.7 | LGALS2 | 2.13 | PRPS1 | 2.7.35 | TBL1Y | 3.17.65 |
| COX5A | 2.7 | ACE | 2.13 | IGFBP3 | 2.7.35 | OR1E2 | 3.17.65 |
| CCT8 | 2.7 | CAPZA3 | 2.13 | TIMM13 | 2.7.35 | OR4K15 | 3.17.65 |
| UBTD1 | 2.7 | NUAK2 | 2.13 | SLCO2A1 | 2.7.35 | MAGEA8 | 3.17.65 |
| JMJD8 | 2.7 | TMEM129 | 2.13 | DOK1 | 2.7.35 | FGF16 | 3.17.65 |
| SERF2 | 2.7 | CXCL2 | 2.13 | OR6P1 | 2.7.35 | MRGPRX3 | 3.17.65 |
| PNPLA2 | 2.7 | DMPK | 2.13 | OR7A17 | 2.7.35 | MAGEA4 | 3.17.65 |
| SYNE4 | 2.7 | CCDC124 | 2.13 | PDZD11 | 2.7.35 | KRTAP15-1 | 3.17.65 |
| OAF | 2.7 | ZBTB7C | 2.13 | TAGLN | 2.7.35 | TRIM43 | 3.17.65 |
| PRPS1 | 2.7 | DEFB134 | 2.13 | HNRNPA2B1 | 2.7.35 | EPHA2 | 3.17.65 |
| IGFBP3 | 2.7 | TREX1 | 2.13 | ORIG1 | 2.7.35 | RAB40AL | 3.17.65 |
| TIMM13 | 2.7 | UGT1A6 | 2.13 | ACTA2 | 2.7.35 | WNK3 | 3.17.66 |
| GPR139 | 2.7 | METRN | 2.13 | SNU13 | 2.7.35 | KLHL4 | 3.17.66 |
| SLCO2A1 | 2.7 | P4HTM | 2.13 | FGFBP2 | 2.7.35 | FAM9B | 3.17.66 |
| TUBG2 | 2.7 | SLC25A1 | 2.13 | CCDC167 | 2.7.35 | GLRA2 | 3.17.66 |
| PLAU | 2.7 | RAB20 | 2.13 | MIDN | 2.7.35 | COL4A5 | 3.17.66 |
| OR4P4 | 2.7 | PILRB | 2.13 | SSBP1 | 2.7.35 | CNKSR2 | 3.17.66 |
| ADAM15 | 2.7 | BOP1 | 2.13 | THBD | 2.7.35 | FAM133A | 3.17.66 |
| DOK1 | 2.7 | GNG3 | 2.13 | CT62 | 2.7.35 | XK | 3.17.66 |
| OR6P1 | 2.7 | TAC4 | 2.13 | EMC7 | 2.7.35 | MCF2 | 3.17.66 |
| OR7A17 | 2.7 | C19orf38 | 2.13 | HCN4 | 2.7.35 | SCML2 | 3.17.66 |
| UROC1 | 2.7 | PNPO | 2.13 | DNAJC3 | 2.7.35 | LANCL3 | 3.17.66 |
| PDZD11 | 2.7 | SLC7A4 | 2.13 | ADARB2 | 2.7.35 | RNF128 | 3.17.66 |
| TAGLN | 2.7 | CYP26C1 | 2.13 | DES | 2.7.35 | FAM9C | 3.17.66 |
| TAF13 | 2.7 | CHMP6 | 2.13 | BCAN | 2.7.35 | SLC6A14 | 3.17.66 |
| HNRNPA2B1 | 2.7 | PARD6A | 2.13 | TWF2 | 2.7.35 | AGTR2 | 3.17.66 |
| OR1G1 | 2.7 | DCXR | 2.13 | TPM2 | 2.7.35 | PCDH11X | 3.17.66 |
| SLC35C1 | 2.7 | P2RY11 | 2.13 | CHIC2 | 2.7.35 | DCAF8L1 | 3.17.66 |
| STK17B | 2.7 | DEXI | 2.13 | HIPK3 | 2.7.35 | LRCH2 | 3.17.66 |
| ACTA2 | 2.7 | MPC1L | 2.13 | FKBP2 | 2.7.35 | TMSB4Y | 3.17.66 |
| MTCH1 | 2.7 | GTF2IRD2B | 2.13 | UBAP2L | 2.7.35 | ASB11 | 3.17.66 |
| DLX4 | 2.7 | UGT2A2 | 2.13 | AKR1C2 | 2.7.35 | ZIC3 | 3.17.66 |
| PRKCSH | 2.7 | KRTAP5-9 | 2.13 | PLIN3 | 2.7.35 | CFAP47 | 3.17.66 |
| SNU13 | 2.7 | BIK | 2.13 | CENPM | 2.7.35 | PGP | 3.17.66 |
| AKT1 | 2.7 | DEFA6 | 2.13 | TAPBP | 2.7.35 | PHKA1 | 3.17.66 |
| LSP1 | 2.7 | SPRR2F | 2.13 | CYP2A13 | 2.7.35 | HMGN5 | 3.17.66 |
| SDC1 | 2.7 | ARHGAP11B | 2.13 | CST9 | 2.7.35 | CCDC160 | 3.17.66 |
| PXN | 2.7 | FLG2 | 2.14 | CT55 | 2.7.35 | POF1B | 3.17.66 |
| F8A1 | 2.7 | FLG | 2.14 | RRBP1 | 2.7.35 | PAGE5 | 3.17.66 |
| FGFBP2 | 2.7 | KRT1 | 2.14 | CUL1 | 2.7.35 | PAGE4 | 3.17.66 |
| CCDC167 | 2.7 | KRT2 | 2.14 | DUSP23 | 2.7.35 | CT83 | 3.17.66 |
| MIDN | 2.7 | DSP | 2.14 | CKB | 2.7.35 | RGPD3 | 3.17.66 |
| SSBP1 | 2.7 | KRT5 | 2.14 | ARL8A | 2.7.35 | Clorf146 | 3.17.66 |
| THBD | 2.7 | KRT10 | 2.14 | RUFY1 | 2.7.35 | XAGE2 | 3.17.66 |
| NLRP5 | 2.7 | KRT14 | 2.14 | TAAR5 | 2.7.35 | CSAG1 | 3.17.66 |
| OR6B1 | 2.7 | CASP14 | 2.14 | CHMP1A | 2.7.35 | CLDN23 | 3.17.66 |
| MEA1 | 2.7 | DSG1 | 2.14 | CPZ | 2.7.35 | JOSD2 | 3.17.66 |
| CT62 | 2.7 | SBSN | 2.14 | HSPA9 | 2.7.35 | FOXQ1 | 3.17.66 |
| TMEM184B | 2.7 | SFN | 2.14 | SPINK4 | 2.7.35 | NRK | 3.17.66 |
| CAPN15 | 2.7 | KRTDAP | 2.14 | MLF2 | 2.7.35 | IL1RAPL2 | 3.17.66 |
| EMC7 | 2.7 | DMKN | 2.14 | OR51I2 | 2.7.35 | LPAR4 | 3.17.66 |
| HBEGF | 2.7 | PKP1 | 2.14 | GPR45 | 2.7.35 | CHIC1 | 3.17.66 |
| HCN4 | 2.7 | CALML5 | 2.14 | IFNGR1 | 2.7.35 | FAM9A | 3.17.66 |
| DNAJC3 | 2.7 | CDHR1 | 2.14 | C10orf71 | 2.7.35 | TNMD | 3.17.66 |
| PLOD1 | 2.7 | LYPD3 | 2.14 | LEFTY1 | 2.7.35 | CXorf58 | 3.17.66 |
| ADARB2 | 2.7 | TRIM29 | 2.14 | TIMM9 | 2.7.35 | XAGE5 | 3.17.66 |
| DES | 2.7 | NCCRP1 | 2.14 | GABARAPL2 | 2.7.35 | RANBP1 | 3.17.66 |
| BCAN | 2.7 | DSC1 | 2.14 | CD164L2 | 2.7.35 | LUZP4 | 3.17.66 |
| TWF2 | 2.7 | KLK5 | 2.14 | SSBP4 | 2.7.35 | PAGE3 | 3.17.66 |
| TPM2 | 2.7 | ALDH3B2 | 2.14 | SPANXN1 | 2.7.35 | TNFRSF18 | 3.17.66 |
| NACC1 | 2.7 | LY6K | 2.14 | CDC123 | 2.7.35 | MTMR10 | 3.17.66 |
| ELL | 2.7 | LY6D | 2.14 | MYOG | 2.7.35 | DRP2 | 3.17.67 |
| TEX22 | 2.7 | FGFR3 | 2.14 | MAP7D1 | 2.7.35 | GYG2 | 3.17.67 |
| CHIC2 | 2.7 | WFDC5 | 2.14 | C2CD4D | 2.7.35 | GABRQ | 3.17.67 |
| HIPK3 | 2.7 | PSAPL1 | 2.14 | HDAC10 | 2.7.35 | PCYT1B | 3.17.67 |
| MRPS26 | 2.7 | TSNARE1 | 2.14 | ZNF703 | 2.7.35 | SYP | 3.17.67 |
| SNAPC2 | 2.7 | HID1 | 2.14 | MAPK11 | 2.7.35 | RS1 | 3.17.67 |
| POMGNT2 | 2.7 | PKP3 | 2.14 | CYGB | 2.7.35 | MAP3K15 | 3.17.67 |
| FAM83C | 2.7 | ASPRVI | 2.14 | HOXD11 | 2.7.35 | XKRX | 3.17.67 |
| FKBP2 | 2.7 | PTK6 | 2.14 | GRK1 | 2.7.35 | CACNAIF | 3.17.67 |
| UBAP2L | 2.7 | GPIHBP1 | 2.14 | FAM90A26 | 2.7.35 | GABRA3 | 3.17.67 |
| DOTIL | 2.7 | EPPK1 | 2.14 | TBX20 | 2.7.35 | TCEAL5 | 3.17.67 |
| RAB11FIP5 | 2.7 | LCE1C | 2.14 | GADD45GIP1 | 2.7.35 | AWAT2 | 3.17.67 |
| WBP1 | 2.7 | PF4V1 | 2.14 | SRSF5 | 2.7.35 | RIBC1 | 3.17.67 |
| AKR1C2 | 2.7 | MIB2 | 2.14 | CCL21 | 2.7.35 | GJB1 | 3.17.67 |
| PLIN4 | 2.7 | ZNF750 | 2.14 | PRAMEF17 | 2.7.35 | PABPC1L2A | 3.17.67 |
| PLIN3 | 2.7 | KRTAP5-10 | 2.14 | XAGE3 | 2.7.35 | AKAP4 | 3.17.67 |
| AAMP | 2.7 | PERP | 2.14 | PAM | 2.7.35 | SSX5 | 3.17.67 |
| CENPM | 2.7 | Clorf68 | 2.14 | CRABP1 | 2.7.35 | CATSPERD | 3.17.67 |
| SCD | 2.7 | KLK7 | 2.14 | POTEI | 2.7.35 | CNGA2 | 3.17.67 |
| TTC9B | 2.7 | KPRP | 2.14 | LCE1E | 2.7.35 | GDPD2 | 3.17.67 |
| TAPBP | 2.7 | SLURP1 | 2.14 | OR7G3 | 2.7.35 | POU3F4 | 3.17.67 |
| GPD1 | 2.7 | FAM83G | 2.14 | OR5A1 | 2.7.35 | ESX1 | 3.17.67 |
| CROCC2 | 2.7 | ZAR1 | 2.14 | ADAM32 | 2.7.35 | SLC30A2 | 3.17.67 |
| SOX3 | 2.7 | ALOX12B | 2.14 | DCAF12L2 | 2.7.35 | EPHA8 | 3.17.67 |
| SERPINH1 | 2.7 | CBX8 | 2.14 | OR11L1 | 2.7.35 | SATL1 | 3.17.67 |
| CYP2A13 | 2.7 | RAB25 | 2.14 | TMEM238 | 2.7.35 | CLDN2 | 3.17.67 |
| CST9 | 2.7 | LCE1A | 2.14 | TMEM95 | 2.7.35 | COL4A6 | 3.17.67 |
| CT55 | 2.7 | PEMT | 2.14 | RTP2 | 2.7.35 | ITGB1BP2 | 3.17.67 |
| GSTT1 | 2.7 | LPCAT1 | 2.14 | AVPR2 | 2.7.35 | SLC38A5 | 3.17.67 |
| RRBP1 | 2.7 | LCE2D | 2.14 | S100G | 2.7.35 | OR10H2 | 3.17.67 |
| CUL1 | 2.7 | CD22 | 2.16 | KRBA2 | 2.7.35 | ARHGAP36 | 3.17.67 |
| LRRC3C | 2.7 | RAC2 | 2.16 | LMNA | 2.7.35 | OR51D1 | 3.17.67 |
| DUSP23 | 2.7 | CXCR4 | 2.16 | GNRH2 | 2.7.35 | SPANXB1 | 3.17.67 |
| MEOX1 | 2.7 | SPOCK2 | 2.16 | GGN | 2.7.35 | CITED1 | 3.17.67 |
| CKB | 2.7 | TCF7 | 2.16 | MT1X | 2.7.35 | COMTD1 | 3.17.67 |
| GAPDHS | 2.7 | IL2RG | 2.16 | CCL17 | 2.7.35 | SOHLH1 | 3.17.67 |
| FASTK | 2.7 | CXCL13 | 2.16 | PRG2 | 2.7.35 | TSNAXIP1 | 3.17.67 |
| ARL8A | 2.7 | CD2 | 2.16 | RARRES2 | 2.7.35 | PLPPR3 | 3.17.67 |
| RUFY1 | 2.7 | CD19 | 2.16 | C8orf58 | 2.7.35 | SHROOM2 | 3.17.67 |
| FABP4 | 2.7 | MS4A1 | 2.16 | PODXL | 2.7.35 | CATIP | 3.17.67 |
| TAAR5 | 2.7 | CD52 | 2.16 | OBP2A | 2.7.35 | MAGEE1 | 3.17.67 |
| CHMP1A | 2.7 | NUP210 | 2.16 | POTEG | 2.7.35 | ERAS | 3.17.67 |
| DAGLA | 2.7 | IL2RB | 2.16 | OR4F4 | 2.7.35 | MAGEB2 | 3.17.67 |
| CPZ | 2.7 | ATP2A3 | 2.16 | HIGD1B | 2.7.35 | MAGEE2 | 3.17.67 |
| HSPA9 | 2.7 | CCR7 | 2.16 | ADAP1 | 2.7.35 | RGN | 3.17.67 |
| ABRACL | 2.7 | FCMR | 2.16 | CCDC74A | 2.7.35 | MAGEA1 | 3.17.67 |
| SPINK4 | 2.7 | IL27RA | 2.16 | HSPB7 | 2.7.35 | TMEM221 | 3.17.67 |
| GLI4 | 2.7 | SPIB | 2.16 | TUBB2A | 2.7.35 | ART1 | 3.17.67 |
| TSR3 | 2.7 | CBFA2T3 | 2.16 | FAM43B | 2.7.35 | TEX28 | 3.17.67 |
| MLF2 | 2.7 | CD37 | 2.16 | OCM | 2.7.35 | LRIT1 | 3.17.67 |
| B4GALNT4 | 2.7 | CXCL1 | 2.16 | PIK3R2 | 2.7.35 | MC5R | 3.17.67 |
| OR51A4 | 2.7 | TMC8 | 2.16 | TAL2 | 2.7.35 | FAM47C | 3.17.67 |
| INPP5E | 2.7 | CD3D | 2.16 | DEFB135 | 2.7.35 | DGAT2L6 | 3.17.67 |
| CIDEC | 2.7 | SELL | 2.16 | YWHAQ | 2.7.35 | MAGEA3 | 3.17.67 |
| RBM42 | 2.7 | RASAL3 | 2.16 | RTN4 | 2.7.35 | BRF2 | 3.17.67 |
| RAMP2 | 2.7 | MMP9 | 2.16 | PHACTR2 | 2.7.35 | WFIKKN2 | 3.17.67 |
| RRP7A | 2.7 | FOXP3 | 2.16 | ANXA5 | 2.7.35 | OCM2 | 3.17.67 |
| OR51I2 | 2.7 | IL21R | 2.16 | ELK3 | 2.7.35 | GATA1 | 3.17.67 |
| OR4S2 | 2.7 | IL7R | 2.16 | RUNX1 | 2.7.35 | MAGEB16 | 3.17.67 |
| GPR45 | 2.7 | FCER2 | 2.16 | TUBA1A | 2.7.35 | CCDC70 | 3.17.67 |
| IFNGR1 | 2.7 | CD5 | 2.16 | MORF4L1 | 2.7.35 | GDF10 | 3.17.67 |
| TNFRSF12A | 2.7 | SIT1 | 2.16 | CHP1 | 2.7.35 | F10 | 3.17.67 |
| CTSK | 2.7 | IGFL1 | 2.16 | THRAP3 | 2.7.35 | SNAI3 | 3.17.67 |
| C10orf71 | 2.7 | KLHDC7B | 2.16 | EID1 | 2.7.35 | SPANXN5 | 3.17.67 |
| LEFTY1 | 2.7 | CD7 | 2.16 | PDLIM1 | 2.7.35 | RAB41 | 3.17.67 |
| TIMM9 | 2.7 | VPREB3 | 2.16 | ITPRIPL2 | 2.7.35 | TCF23 | 3.17.67 |
| MUC1 | 2.7 | TNFRSF13C | 2.16 | ENAH | 2.7.35 | BMP15 | 3.17.67 |
| GABARAPL2 | 2.7 | S1PR4 | 2.16 | CD93 | 2.7.35 | PPP1R3F | 3.17.67 |
| CD164L2 | 2.7 | TCL1A | 2.16 | PRDX6 | 2.7.35 | FAM47A | 3.17.67 |
| SSBP4 | 2.7 | CD79B | 2.16 | RRAGA | 2.7.35 | RNASE7 | 3.17.67 |
| SPANXN1 | 2.7 | PAX5 | 2.16 | WSB1 | 2.7.35 | GPR173 | 3.17.67 |
| NINJ2 | 2.7 | DUSP2 | 2.16 | LIX1L | 2.7.35 | ZCCHC18 | 3.17.67 |
| GABRD | 2.7 | DENND2D | 2.16 | ARRDC3 | 2.7.35 | PSG4 | 3.17.67 |
| TWIST1 | 2.7 | MAP4K1 | 2.16 | PDGFRA | 2.7.35 | PIP5KL1 | 3.17.67 |
| OR4C11 | 2.7 | CD3G | 2.16 | RTN3 | 2.7.35 | MLNR | 3.17.67 |
| GFER | 2.7 | ZC3H12D | 2.16 | NUB1 | 2.7.35 | PAGE1 | 3.17.67 |
| CDC123 | 2.7 | LDHB | 2.16 | ISCU | 2.7.35 | SPANXN3 | 3.17.67 |
| MYOG | 2.7 | ZAP70 | 2.16 | HNRNPUL2 | 2.7.35 | WDR38 | 3.17.67 |
| AGAP9 | 2.7 | DEFB114 | 2.16 | PAIP2 | 2.7.35 | OR9I1 | 3.17.67 |
| MAP7D1 | 2.7 | CCL19 | 2.16 | EBF1 | 2.7.35 | BCKDHA | 3.17.67 |
| C2CD4D | 2.7 | ARHGEF35 | 2.16 | FBN1 | 2.7.35 | TLR9 | 3.17.67 |
| ADIPOQ | 2.7 | WNT11 | 2.16 | ZNF217 | 2.7.35 | SPDYE5 | 3.17.67 |
| PARK7 | 2.7 | SPINK14 | 2.16 | HNRNPF | 2.7.35 | KIAA1210 | 3.17.68 |
| HDAC10 | 2.7 | USP9Y | 3.17 | UGP2 | 2.7.35 | ARR3 | 3.17.68 |
| ZNF703 | 2.7 | UTY | 3.17 | GSTP1 | 2.7.35 | NXF3 | 3.17.68 |
| SEMA6B | 2.7 | DDX3Y | 3.17 | NOLC1 | 2.7.35 | FRMPD3 | 3.17.68 |
| MAPK11 | 2.7 | KDM5D | 3.17 | TXLNA | 2.7.35 | ATP2B3 | 3.17.68 |
| CYGB | 2.7 | RPS4Y1 | 3.17 | FBXW5 | 2.7.35 | ITIH6 | 3.17.68 |
| DPEP1 | 2.7 | NLGN4Y | 3.17 | LMBRD1 | 2.7.35 | SCNN1A | 3.17.68 |
| HOXD11 | 2.7 | PTCHD1 | 3.17 | CXXC5 | 2.7.35 | SLC22A14 | 3.17.68 |
| GRK1 | 2.7 | WNK3 | 3.17 | ZMAT2 | 2.7.35 | PNCK | 3.17.68 |
| TMEM200B | 2.7 | KLHL4 | 3.17 | MEDAG | 2.7.35 | GPR101 | 3.17.68 |
| MFSD3 | 2.7 | DRP2 | 3.17 | HERPUD2 | 2.7.35 | GPR50 | 3.17.68 |
| HRCT1 | 2.7 | IGSF1 | 3.17 | TAF7 | 2.7.35 | ASB12 | 3.17.68 |
| FAM90A26 | 2.7 | FAM9B | 3.17 | HNRNPDL | 2.7.35 | TRPM5 | 3.17.68 |
| TBX20 | 2.7 | AFF2 | 3.17 | UBE2D2 | 2.7.35 | LRRC8E | 3.17.68 |
| GADD45GIP1 | 2.7 | GUCY2F | 3.17 | GNPTG | 2.7.35 | OR13H1 | 3.17.68 |
| SRSF5 | 2.7 | DCX | 3.17 | STK25 | 2.7.35 | KCND1 | 3.17.68 |
| CCL21 | 2.7 | CREG1 | 3.17 | PSMF1 | 2.7.35 | ACTL7A | 3.17.68 |
| PRAMEF17 | 2.7 | ACE2 | 3.17 | PACSIN2 | 2.7.35 | RNF186 | 3.17.68 |
| XAGE3 | 2.7 | ADGRG4 | 3.17 | TTC1 | 2.7.35 | CXorf65 | 3.17.68 |
| PAM | 2.7 | DGKK | 3.17 | ETS2 | 2.7.35 | EPO | 3.17.68 |
| SYDE1 | 2.7 | GLRA2 | 3.17 | TES | 2.7.35 | FAM187B | 3.17.68 |
| CRABP1 | 2.7 | GYG2 | 3.17 | UGDH | 2.7.35 | CCKBR | 3.17.68 |
| RASD1 | 2.7 | F9 | 3.17 | GINM1 | 2.7.35 | DMRT3 | 3.17.68 |
| POTEI | 2.7 | BRS3 | 3.17 | MAGEH1 | 2.7.35 | ANKRD24 | 3.17.68 |
| P2RX1 | 2.7 | CAPN6 | 3.17 | TAF12 | 2.7.35 | OR52I1 | 3.17.68 |
| LCE1E | 2.7 | COL4A5 | 3.17 | PSMD4 | 2.7.35 | MSGN1 | 3.17.68 |
| OR7G3 | 2.7 | DACH2 | 3.17 | PARP11 | 2.7.35 | TPSG1 | 3.17.68 |
| OR5A1 | 2.7 | USP26 | 3.17 | PFKFB1 | 2.7.35 | XPNPEP2 | 3.17.68 |
| NENF | 2.7 | KIAA1210 | 3.17 | TMEM14C | 2.7.35 | ZNF625 | 3.17.68 |
| ADAM32 | 2.7 | CNKSR2 | 3.17 | SIRT2 | 2.7.35 | ABHD1 | 3.17.68 |
| DCAF12L2 | 2.7 | PHEX | 3.17 | PKD2L1 | 2.7.35 | SMIM10L2A | 3.17.68 |
| IFNA10 | 2.7 | FRMPD4 | 3.17 | SRSF7 | 2.7.35 | SNX32 | 3.17.68 |
| OR11L1 | 2.7 | GABRQ | 3.17 | ATG9A | 2.7.35 | GGTLC1 | 3.17.68 |
| TMEM238 | 2.7 | ARSF | 3.17 | C1QBP | 2.7.35 | OR2Y1 | 3.17.68 |
| TMEM95 | 2.7 | ZC3H12B | 3.17 | MAP1B | 2.7.35 | CT47B1 | 3.17.68 |
| RTP2 | 2.7 | FAM133A | 3.17 | PLAC9 | 2.7.35 | IL17REL | 3.17.68 |
| FZD9 | 2.7 | XK | 3.17 | GNG11 | 2.7.35 | ARL6IP4 | 3.17.68 |
| NEU2 | 2.7 | MCF2 | 3.17 | CFI | 2.7.35 | CSMD3 | 3.18.70 |
| AVPR2 | 2.7 | TKTL1 | 3.17 | ECH1 | 2.7.35 | CDH9 | 3.18.70 |
| S100G | 2.7 | SCML2 | 3.17 | RBMX2 | 2.7.35 | SYCP1 | 3.18.70 |
| PODNL1 | 2.7 | LANCL3 | 3.17 | C1GALT1C1 | 2.7.35 | MGAT4C | 3.18.70 |
| LCN2 | 2.7 | PCYT1B | 3.17 | IFFO1 | 2.7.35 | RPS6KA6 | 3.18.70 |
| KRBA2 | 2.7 | ZFY | 3.17 | POLR2I | 2.7.35 | CCDC73 | 3.18.70 |
| OR52N5 | 2.7 | RNF128 | 3.17 | CDO1 | 2.7.35 | SYCP2 | 3.18.70 |
| LMNA | 2.7 | MAP7D2 | 3.17 | EMD | 2.7.35 | TECRL | 3.18.70 |
| GNRH2 | 2.7 | FAM9C | 3.17 | IQCJ | 2.7.35 | CADM2 | 3.18.70 |
| AMHR2 | 2.7 | TMEM255A | 3.17 | OR4Q3 | 2.7.35 | LRRIQ3 | 3.18.70 |
| GGN | 2.7 | AR | 3.17 | SF3A2 | 2.7.35 | GABRG1 | 3.18.70 |
| MT1X | 2.7 | DDX53 | 3.17 | NRBF2 | 2.7.35 | CDH7 | 3.18.70 |
| CCL17 | 2.7 | SLC6A14 | 3.17 | C11orf96 | 2.7.35 | SPOCK3 | 3.18.70 |
| FBXL14 | 2.7 | AGTR2 | 3.17 | GADD45A | 2.7.35 | GLIPRIL2 | 3.18.70 |
| PSPN | 2.7 | PAK3 | 3.17 | SCGB2A2 | 2.7.35 | FSTL5 | 3.18.70 |
| TTLL12 | 2.7 | PCDH11X | 3.17 | MED18 | 2.7.35 | LRRIQ1 | 3.18.70 |
| PRG2 | 2.7 | FOXR2 | 3.17 | PYURF | 2.7.35 | TMEFF2 | 3.18.70 |
| RARRES2 | 2.7 | SSX3 | 3.17 | TPSB2 | 2.7.35 | SI | 3.18.70 |
| C8orf58 | 2.7 | ARR3 | 3.17 | CBX4 | 2.7.35 | MGAT4D | 3.18.70 |
| NICN1 | 2.7 | SYP | 3.17 | OR10A2 | 2.7.35 | EPHA5 | 3.18.70 |
| PODXL | 2.7 | RS1 | 3.17 | PCEDIA | 2.7.35 | STATH | 3.18.70 |
| OBP2A | 2.7 | NOX1 | 3.17 | HOXC12 | 2.7.35 | MYBPC1 | 3.18.70 |
| POTEG | 2.7 | ATP1B4 | 3.17 | JPH2 | 2.7.35 | ZNF711 | 3.18.70 |
| KLK12 | 2.7 | MAP3K15 | 3.17 | RPL10L | 2.7.35 | EPYC | 3.18.70 |
| OR4F4 | 2.7 | GPR119 | 3.17 | LDOC1 | 2.7.35 | PI15 | 3.18.70 |
| HIGD1B | 2.7 | HEPH | 3.17 | KDM4E | 2.7.35 | PRR27 | 3.18.70 |
| ASIC5 | 2.7 | XKRX | 3.17 | TRAPPC12 | 2.7.35 | SNX16 | 3.18.70 |
| ADAP1 | 2.7 | DCAF8L1 | 3.17 | ERICH5 | 2.7.35 | C14orf39 | 3.18.70 |
| NUPR2 | 2.7 | DCAF8L2 | 3.17 | CDC42EP2 | 2.7.35 | ANGPTL3 | 3.18.70 |
| PPP1R27 | 2.7 | LRCH2 | 3.17 | DCTPP1 | 2.7.35 | LUZP2 | 3.18.70 |
| CCDC74A | 2.7 | EGFL6 | 3.17 | ABHD11 | 2.7.35 | SLC6A15 | 3.18.70 |
| HSPB7 | 2.7 | CACNAIF | 3.17 | SOAT2 | 2.7.35 | PLPPR5 | 3.18.70 |
| GOLGA6L4 | 2.7 | NXF3 | 3.17 | RNF113B | 2.7.35 | MICU3 | 3.18.70 |
| TUBB2A | 2.7 | GABRA3 | 3.17 | TCEAL1 | 2.7.35 | GPR22 | 3.18.70 |
| FAM43B | 2.7 | TAF7L | 3.17 | NTSR2 | 2.7.35 | KLRC1 | 3.18.70 |
| PUSL1 | 2.7 | FAM156A | 3.17 | IFI35 | 2.7.35 | OSTN | 3.18.70 |
| OCM | 2.7 | TCEAL5 | 3.17 | ILK | 2.7.35 | CDH10 | 3.18.70 |
| TMEM59L | 2.7 | SAGE1 | 3.17 | ASTL | 2.7.35 | KIF18A | 3.18.70 |
| TBC1D10C | 2.7 | FRMPD3 | 3.17 | CST9L | 2.7.35 | HCN1 | 3.18.70 |
| CYB561D2 | 2.7 | FRMD7 | 3.17 | AGT | 2.7.35 | CSN3 | 3.18.70 |
| RSPH10B2 | 2.7 | EIF1AY | 3.17 | PRDM13 | 2.7.35 | RGS21 | 3.18.70 |
| SCRT1 | 2.7 | ATP2B3 | 3.17 | THTPA | 2.7.35 | CFHR1 | 3.18.70 |
| PIK3R2 | 2.7 | TMEM35A | 3.17 | OR56A4 | 2.7.35 | NECAB1 | 3.18.70 |
| RBP4 | 2.7 | MAGEB3 | 3.17 | DVL1 | 2.7.35 | TAPBPL | 3.18.70 |
| AKAP8 | 2.7 | AWAT2 | 3.17 | KRTAP4-3 | 2.7.35 | FANCB | 3.18.70 |
| TAL2 | 2.7 | GPC3 | 3.17 | CELA2B | 2.7.35 | NOL4 | 3.18.70 |
| DEFB135 | 2.7 | PASD1 | 3.17 | KCNQ2 | 2.7.35 | GJE1 | 3.18.70 |
| CBARP | 2.7 | GPR143 | 3.17 | NPY | 2.7.35 | F13B | 3.18.70 |
| YWHAQ | 2.7 | RIBC1 | 3.17 | SMIM10 | 2.7.35 | MYOT | 3.18.70 |
| RTN4 | 2.7 | GJB1 | 3.17 | SCGB1D4 | 2.7.35 | PLSCR5 | 3.18.70 |
| PHACTR2 | 2.7 | PABPC1L2A | 3.17 | CAMK2N1 | 2.7.35 | RAB3D | 3.18.70 |
| ANXA5 | 2.7 | RBX1 | 3.17 | GAL3ST1 | 2.7.35 | SLCO1B3 | 3.18.70 |
| ELK3 | 2.7 | AKAP4 | 3.17 | HES6 | 2.7.35 | UGT2B7 | 3.18.70 |
| DYNC1H1 | 2.7 | ITIH6 | 3.17 | CACNA1H | 2.7.35 | TRDN | 3.18.70 |
| RUNX1 | 2.7 | TMSB4Y | 3.17 | CLEC14A | 2.7.35 | MYL1 | 3.18.70 |
| TUBA1A | 2.7 | SSX5 | 3.17 | ABHD8 | 2.7.35 | KHDRBS2 | 3.18.70 |
| MORF4L1 | 2.7 | PLP1 | 3.17 | RFPL1 | 2.7.35 | NTS | 3.18.70 |
| CHP1 | 2.7 | CATSPERD | 3.17 | TPTE | 2.7.35 | UGT2B11 | 3.18.70 |
| THRAP3 | 2.7 | ASB11 | 3.17 | C16orf86 | 2.7.35 | TEX11 | 3.18.70 |
| HINRNPC | 2.7 | NLRP2B | 3.17 | ARHGEF18 | 2.7.35 | ZDHHC24 | 3.18.70 |
| HUWE1 | 2.7 | ZIC3 | 3.17 | PTTG1IP | 2.7.36 | VPS37C | 3.18.70 |
| EID1 | 2.7 | CNGA2 | 3.17 | ANXA2 | 2.7.36 | PRX | 3.18.70 |
| PDLIM1 | 2.7 | GDPD2 | 3.17 | PIGT | 2.7.36 | MYEF2 | 3.18.70 |
| ITPRIPL2 | 2.7 | POU3F4 | 3.17 | PEA15 | 2.7.36 | XKR3 | 3.18.70 |
| ENAH | 2.7 | SCNN1A | 3.17 | APLP2 | 2.7.36 | HECTD3 | 3.18.70 |
| CD93 | 2.7 | SLC22A14 | 3.17 | GPNMB | 2.7.36 | GKN1 | 3.18.70 |
| PRDX6 | 2.7 | CFAP47 | 3.17 | LDHA | 2.7.36 | SPO11 | 3.18.70 |
| RRAGA | 2.7 | OTC | 3.17 | TGFBI | 2.7.36 | SGSM2 | 3.18.70 |
| RIN2 | 2.7 | PNCK | 3.17 | PCOLCE | 2.7.36 | SLC37A2 | 3.18.70 |
| WSB1 | 2.7 | ESX1 | 3.17 | GNB1 | 2.7.36 | USP11 | 3.18.70 |
| LIX1L | 2.7 | GPR101 | 3.17 | APP | 2.7.36 | SH3BP1 | 3.18.70 |
| CHMP2A | 2.7 | AKAP14 | 3.17 | SUMO3 | 2.7.36 | HEXIM2 | 3.18.70 |
| CKAP4 | 2.7 | NCBP2L | 3.17 | EFEMP1 | 2.7.36 | ST14 | 3.18.70 |
| ARRDC3 | 2.7 | PABPC1L2B | 3.17 | PSMC | 2.7.36 | PLCD1 | 3.18.70 |
| PDGFRA | 2.7 | SLC30A2 | 3.17 | SURF4 | 2.7.36 | AQP3 | 3.18.70 |
| PRPF6 | 2.7 | PGP | 3.17 | AP2M1 | 2.7.36 | TRMT61A | 3.18.70 |
| RTN3 | 2.7 | PHKA1 | 3.17 | CHMP4B | 2.7.36 | LIPI | 3.18.70 |
| NUB1 | 2.7 | HMGN5 | 3.17 | TMED9 | 2.7.36 | IL5 | 3.18.70 |
| GOLGA3 | 2.7 | GPR50 | 3.17 | YWHAH | 2.7.36 | CAPS | 3.18.70 |
| ISCU | 2.7 | CCDC160 | 3.17 | WBP1L | 2.7.36 | DGKQ | 3.18.70 |
| ZNF362 | 2.7 | ASB12 | 3.17 | PRNP | 2.7.36 | OR10AG1 | 3.18.70 |
| FOXO1 | 2.7 | EPHA8 | 3.17 | ECM1 | 2.7.36 | TRIM48 | 3.18.70 |
| HNRNPUL2 | 2.7 | TRPM5 | 3.17 | YIPF3 | 2.7.36 | IFNA17 | 3.18.70 |
| PAIP2 | 2.7 | LRRC8E | 3.17 | VEGFB | 2.7.36 | FRG2B | 3.18.70 |
| EBF1 | 2.7 | POF1B | 3.17 | GPR108 | 2.7.36 | AREG | 3.18.70 |
| FBN1 | 2.7 | PAGE5 | 3.17 | VKORCI | 2.7.36 | SLCO4A1 | 3.18.70 |
| ZNF217 | 2.7 | OR13H1 | 3.17 | TMEM109 | 2.7.36 | PLEKHJ1 | 3.18.70 |
| HNRNPF | 2.7 | KLHL34 | 3.17 | GLG1 | 2.7.36 | KRTAP21-3 | 3.18.70 |
| UGP2 | 2.7 | PLAC1 | 3.17 | TNC | 2.7.36 | POLRMT | 3.18.70 |
| PPP1CA | 2.7 | SATL 1 | 3.17 | PSMC5 | 2.7.36 | DEFB112 | 3.18.70 |
| TCF25 | 2.7 | CLDN2 | 3.17 | PDIA3 | 2.7.36 | TMSB15B | 3.18.70 |
| RARA | 2.7 | SLITRK2 | 3.17 | ACTRIA | 2.7.36 | NDST3 | 3.18.70 |
| GSTP1 | 2.7 | KCND1 | 3.17 | NINJ1 | 2.7.36 | TREML1 | 3.18.70 |
| NOLC1 | 2.7 | ACTL7A | 3.17 | EPHX1 | 2.7.36 | SPINK1 | 3.18.70 |
| TXLNA | 2.7 | COL4A6 | 3.17 | FURIN | 2.7.36 | FOSL1 | 3.18.70 |
| FBXW5 | 2.7 | ITGB1BP2 | 3.17 | SERINC3 | 2.7.36 | ALG12 | 3.18.70 |
| LMBRD1 | 2.7 | SLC38A5 | 3.17 | ITGB5 | 2.7.36 | FBXO2 | 3.18.70 |
| DHRS3 | 2.7 | ADRA2B | 3.17 | BHLHE40 | 2.7.36 | TEX12 | 3.18.70 |
| ARF3 | 2.7 | OR10H2 | 3.17 | C1RL | 2.7.36 | HLA-DRB5 | 3.18.70 |
| CXXC5 | 2.7 | SLC9A3R2 | 3.17 | MAF1 | 2.7.36 | LRRC29 | 3.18.70 |
| ATRAID | 2.7 | PAGE4 | 3.17 | CALU | 2.7.36 | C1QL4 | 3.18.70 |
| B4GALT5 | 2.7 | ARHGAP36 | 3.17 | KDELR1 | 2.7.36 | OGFOD2 | 3.18.70 |
| ZMAT2 | 2.7 | OR51D1 | 3.17 | TMBIM1 | 2.7.36 | REEP6 | 3.18.70 |
| GABARAPL1 | 2.7 | PCDH11Y | 3.17 | PLD3 | 2.7.36 | EEF2KMT | 3.18.70 |
| MEDAG | 2.7 | TEX13B | 3.17 | SFRP1 | 2.7.36 | CFHR3 | 3.18.70 |
| HERPUD2 | 2.7 | SPANXB1 | 3.17 | LRP10 | 2.7.36 | NPIPB11 | 3.18.70 |
| TAF7 | 2.7 | CITED1 | 3.12 | TNFAIP1 | 2.7.36 | POTEB3 | 3.18.70 |
| DNAJB1 | 2.7 | RNF186 | 3.17 | ANKH | 2.7.36 | CARD9 | 3.18.70 |
| HNRNPDL | 2.7 | FMRINB | 3.17 | CIZ1 | 2.7.36 | VCX3A | 3.18.70 |
| TALDO1 | 2.7 | NECAB2 | 3.17 | SLC35F6 | 2.7.36 | FUOM | 3.18.70 |
| UBE2D2 | 2.7 | MAGEB1 | 3.17 | ERRFI1 | 2.7.36 | LRRC70 | 3.18.70 |
| GNPTG | 2.7 | TCEAL2 | 3.17 | DAPK3 | 2.7.36 | SAC3D1 | 3.18.70 |
| STK25 | 2.7 | CXorf65 | 3.17 | UBAP1 | 2.7.36 | CYLC2 | 3.18.70 |
| SHC1 | 2.7 | COMTD1 | 3.17 | UBE2L3 | 2.7.36 | CST1 | 3.18.70 |
| PSMF1 | 2.7 | SOHLH1 | 3.17 | CD276 | 2.7.36 | NBPF4 | 3.18.70 |
| PACSIN2 | 2.7 | TSNAXIP1 | 3.17 | MXRA5 | 2.7.36 | EPHA7 | 3.18.70 |
| Clorf43 | 2.7 | EPO | 3.17 | TP53I11 | 2.7.36 | OR5H15 | 3.18.70 |
| NT5E | 2.7 | PNMA3 | 3.17 | NPTN | 2.7.36 | CTNNBIP1 | 3.18.70 |
| CSGALNACT1 | 2.7 | FAM187B | 3.17 | NDUFB10 | 2.7.36 | GGT1 | 3.18.70 |
| TTC1 | 2.7 | VGLL1 | 3.17 | TUBG1 | 2.7.36 | KRTAP20-4 | 3.18.70 |
| FCGRT | 2.7 | CCKBR | 3.12 | GLTP | 2.7.36 | TSSK6 | 3.18.70 |
| ETS2 | 2.7 | PLPPR3 | 3.17 | ID2 | 2.7.36 | E2F1 | 3.18.70 |
| TES | 2.7 | RNF112 | 3.17 | COPS7A | 2.7.36 | COX7B2 | 3.18.70 |
| EIF4H | 2.7 | PNMA5 | 3.17 | CLIC5 | 2.7.36 | SLITRK5 | 3.18.70 |
| UGDH | 2.7 | CT83 | 3.17 | SERF2 | 2.7.36 | HFM1 | 3.18.70 |
| GINM1 | 2.7 | UNC5A | 3.17 | PLAU | 2.7.36 | TMPRSS11A | 3.18.70 |
| POLDIP2 | 2.7 | DMRT3 | 3.1 | MTCH1 | 2.7.36 | CYLC1 | 3.18.70 |
| MAGEH1 | 2.7 | SHROOM2 | 3.17 | DLX4 | 2.7.36 | PLCZ1 | 3.18.70 |
| CIB1 | 2.7 | CATIP | 3.17 | LSP1 | 2.7.36 | CFHR4 | 3.18.70 |
| DEDD2 | 2.7 | SYTL5 | 3.17 | PXN | 2.7.36 | ZSWIM2 | 3.18.70 |
| TAF12 | 2.7 | C16orf82 | 3.17 | MEA1 | 2.7.36 | GLIPRIL1 | 3.18.70 |
| TIMM23 | 2.7 | MAGEE1 | 3.15 | HBEGF | 2.7.36 | CDK1 | 3.18.70 |
| PSMD4 | 2.7 | ERAS | 3.12 | PLOD1 | 2.7.36 | GRB14 | 3.18.70 |
| KIAA0040 | 2.7 | MAGEB2 | 3.17 | ELL | 2.7.36 | ZCWPW2 | 3.18.70 |
| PARP11 | 2.7 | ANKRD24 | 3.17 | MRPS26 | 2.7.36 | OR5M3 | 3.18.70 |
| PFKFB1 | 2.7 | LCN9 | 3.17 | SNAPC2 | 2.7.36 | LRRC72 | 3.18.70 |
| TMEM14C | 2.7 | RGPD3 | 3.17 | POMGNT2 | 2.7.36 | DDA1 | 3.18.70 |
| NME4 | 2.7 | NRN1L | 3.17 | RAB11FIP5 | 2.7.36 | FABP2 | 3.18.70 |
| COL4A1 | 2.7 | MAGEE2 | 3.17 | AAMP | 2.7.36 | MAEL | 3.18.70 |
| SIRT2 | 2.7 | MADCAM1 | 3.17 | SCD | 2.7.36 | DDX51 | 3.18.70 |
| GLIPR2 | 2.7 | GH2 | 3.17 | SERPINH1 | 2.7.36 | RIPK3 | 3.18.70 |
| PKD2L1 | 2.7 | GTSF1L | 3.12 | GSTT1 | 2.7.36 | CCDC137 | 3.18.70 |
| SRSF7 | 2.7 | RGN | 3.17 | FASTK | 2.7.36 | STARD10 | 3.18.70 |
| DCTN2 | 2.7 | OR5211 | 3.17 | GLI4 | 2.7.36 | DEFB113 | 3.18.70 |
| TXN2 | 2.7 | ARHGEF25 | 3.17 | OR51A4 | 2.7.36 | BORCS6 | 3.18.70 |
| HYAL2 | 2.7 | MAGEA1 | 3.17 | RAMP2 | 2.7.36 | PMCH | 3.18.70 |
| ATG9A | 2.7 | MSGN1 | 3.17 | TNFRSF12A | 2.7.36 | CCDC179 | 3.18.70 |
| C1QBP | 2.7 | C1orf146 | 3.17 | CTSK | 2.7.36 | ZNF404 | 3.18.70 |
| GUK1 | 2.7 | PAGE2 | 3.17 | AGAP9 | 2.7.36 | SLC19A1 | 3.18.70 |
| TPRG1L | 2.7 | TMEM221 | 3.17 | PARK7 | 2.7.36 | GCSH | 3.18.70 |
| MAP1B | 2.7 | DLL3 | 3.17 | SEMA6B | 2.7.36 | FOXJ1 | 3.18.70 |
| PLAC9 | 2.7 | TP53TG3D | 3.17 | MFSD3 | 2.7.36 | USP17L1 | 3.18.70 |
| HNF4A | 2.7 | ART1 | 3.17 | RASD1 | 2.7.36 | NUTM2B | 3.18.70 |
| ADGRA2 | 2.7 | XAGE2 | 3.17 | P2RX1 | 2.7.36 | KRTAP12-3 | 3.18.70 |
| BEX3 | 2.7 | MAGEA12 | 3.17 | NENF | 2.7.36 | LBX2 | 3.18.70 |
| GNG11 | 2.7 | TEX28 | 3.17 | FBXL14 | 2.7.36 | TTN | 3.18.71 |
| CFI | 2.7 | AMDHD2 | 3.17 | CYB561D2 | 2.7.36 | NEB | 3.18.71 |
| ECH1 | 2.7 | C5orf49 | 3.17 | RBP4 | 2.7.36 | MYH7 | 3.18.71 |
| PROCR | 2.7 | LRIT1 | 3.17 | AKAP8 | 2.7.36 | MYH1 | 3.18.71 |
| RBMX2 | 2.7 | MC5R | 3.17 | RIN2 | 2.7.36 | CA3 | 3.18.71 |
| SUN2 | 2.7 | VMO1 | 3.17 | CKAP4 | 2.7.36 | CKM | 3.18.71 |
| OSR2 | 2.7 | FAM47C | 3.17 | FOXO1 | 2.7.36 | ACTA1 | 3.18.71 |
| CLUH | 2.7 | DGAT2L6 | 3.17 | PPP1CA | 2.7.36 | MB | 3.18.71 |
| FPGS | 2.7 | MAGEA3 | 3.17 | RARA | 2.7.36 | MYL2 | 3.18.71 |
| C1GALT1C1 | 2.7 | IL4 | 3.17 | DHRS3 | 2.7.36 | TNNI1 | 3.18.71 |
| IFFO1 | 2.7 | CSAG1 | 3.17 | ARF3 | 2.7.36 | ATP2A1 | 3.18.71 |
| POLR2I | 2.7 | BRF2 | 3.17 | ATRAID | 2.7.36 | TNNT1 | 3.18.71 |
| PPIH | 2.7 | ZMAT5 | 3.17 | B4GALT5 | 2.7.36 | XIRP1 | 3.18.71 |
| PKN1 | 2.7 | ENTPD8 | 3.17 | GABARAPL1 | 2.7.36 | ENO3 | 3.18.71 |
| ARF5 | 2.7 | TPSG1 | 3.17 | Clorf43 | 2.7.36 | SMPX | 3.18.71 |
| TMEM147 | 2.7 | WFIKKN2 | 3.17 | NT5E | 2.7.36 | HDAC11 | 3.18.71 |
| PPM1M | 2.7 | OCM2 | 3.17 | CSGALNACT1 | 2.7.36 | TNNC1 | 3.18.71 |
| CRYAB | 2.7 | GATA1 | 3.17 | POLDIP2 | 2.7.36 | TNNT3 | 3.18.71 |
| TAX1BP3 | 2.7 | CLDN23 | 3.17 | CIB1 | 2.7.36 | MYL3 | 3.18.71 |
| CDO1 | 2.7 | JOSD2 | 3.17 | TIMM23 | 2.7.36 | PRR36 | 3.18.71 |
| EMD | 2.7 | PAGE2B | 3.17 | KIAA0040 | 2.7.36 | EEF1A2 | 3.18.71 |
| IQCJ | 2.7 | FOXQ1 | 3.17 | NME4 | 2.7.36 | TCAP | 3.18.71 |
| MAGEC3 | 2.7 | XPNPEP2 | 3.17 | DCTN2 | 2.7.36 | TNNC2 | 3.18.71 |
| OR4Q3 | 2.7 | MAGEB16 | 3.17 | TXN2 | 2.7.36 | MYH2 | 3.18.71 |
| SF3A2 | 2.7 | KRTAP11-1 | 3.17 | HYAL2 | 2.7.36 | MYOZ1 | 3.18.71 |
| NRBF2 | 2.7 | MAGEB18 | 3.17 | GUK1 | 2.7.36 | MYBPC2 | 3.18.71 |
| C11orf96 | 2.7 | HOXA6 | 3.17 | BEX3 | 2.7.36 | PYGM | 3.18.71 |
| GADD45A | 2.7 | CCDC70 | 3.17 | PROCR | 2.7.36 | C1QTNF9B | 3.18.71 |
| RGS16 | 2.7 | GDF10 | 3.17 | TMEM147 | 2.7.36 | TNNI2 | 3.18.71 |
| IDUA | 2.7 | CAMP | 3.17 | PPM1M | 2.7.36 | FITM1 | 3.18.71 |
| SCGB2A2 | 2.7 | F10 | 3.17 | TAX1BP3 | 2.7.36 | DNAAF3 | 3.18.71 |

### Example 7: Molecular Endotypes of RA Patient Whole Blood Sample

A method is carried out to characterize the molecular landscape of patients with rheumatoid arthritis (RA) by analyzing gene expression profiles from whole blood samples. Whole blood samples are collected from 2 patient populations: (1) patients with rheumatoid arthritis (RA) that are incomplete responders (IR) to disease-modifying antirheumatic drug (DMARD) treatment (DMARD-IR), and (2) patients with rheumatoid arthritis (RA) that are incomplete responders (IR) to TNF Inhibitor treatment (TNF-IR).

Full transcriptomic RNA sequencing is carried out on whole blood samples collected from 2 patient populations as compared to healthy patients (control).

In brief, whole blood is collected in PAXgene Blood RNA tubes. After removal of ribosomal RNA and globin transcripts with the Ribo-Zero Globin Removal kit (Illumina), stranded libraries are prepared with the TruSeq Library prep kit (Illumina) and hybridized to a flow cell for sequencing with the Illumina HiSeq platform. Raw RNAseq output counts are log 2 normalized using the R DESeq2 package. The top 5,000 row variance (top5k rowVar) genes determined using standard deviation between samples are retained for further analysis.

The top 5,000 row variance genes are analyzed by a suite of gene expression technologies, including Multiscale Embedded Gene Co-expression Network Analysis (MEGENA) to generate gene coexpression modules which are functionally annotated and correlated to various demographic traits, clinical features, and laboratory assays.

In brief, the MEGENA R package is used to generate a gene coexpression network by inputting the top5k rowVar genes. MEGENA multi-scale clustering analysis (MCA) formed lineages of gene modules followed by identification of densely intraconnected hub genes using multi-scale hub analysis (MHA). Modules are assigned “lineage” names based on their multiscale pedigree from the root MEGENA module. The prcomp package is utilized to perform singular value decomposition and calculate MEGENA module eigengenes (MEs), equivalent to the first principal component calculated amongst the variance of a given MEGENA module. MEGENA MEs are correlated to the numerically encoded sample traits.

A heatmap is generated using ComplexHeatmap visualizing the top sample trait correlations to the MEGENA modules that are significantly correlated to cohort (or cluster). Module gene symbols are used to programmatically query the STRING database and calculate the percentage of genes within a given module predicted to have known protein-protein interactions (PPI) ranging from 0 to 100%.

A gene set variation analysis (GSVA) (GSVA (V1.25.0) R software package) is carried out as a non-parametric, unsupervised method for estimating the variation of pre-defined gene sets over all MEGENA module log 2 gene expression values. Input genes are employed only if the interquartile range (IQR) of their expression across the samples is greater than 0. Enrichment scores (GSVA scores) are calculated non-parametrically using a Kolmogorov Smirnoff (KS)-like random walk statistic. The enrichment scores(ES) are the largest positive and negative random walk deviations from zero, respectively, for a particular sample amongst the module gene set. The GSVA scores are used as input for unsupervised stable k-means clustering, and different disease phenotypes or clusters are identified. GSVA is performed using the significant MEGENA modules as gene signatures.

The MEs of the significant MEGENA modules are correlated to mean gene expression of a given module per patient and visualized using Complex heatmap. The heatmap's columns show different disease phenotypes or clusters are identified after patients are clustered using idealized k-means clustering. The heatmap's rows show various immune cell type and process modules (e.g., modules associated to inflammation, modules associated to cells, modules associated to JAK/TYK2, modules associated to steroids) used to identify the different disease phenotypes/clusters/patient subsets. In brief, the modules, including those for steroid response and JAK/TYK pathways (e.g., steroid gene markers, JAK1, JAK2, and TYK2) can be associated to subsets with enriched treatment targets. Overall, stable k-means clustering of gene coexpression modules effectively segregates patient subsets into different disease phenotypes which may be associated to different treatment targets and/or responsive to different treatments.

### Example 8: Molecular Endotypes of RA Patient Synovium Sample

A method is carried out to characterize the molecular landscape of patients with rheumatoid arthritis (RA) by analyzing gene expression profiles from synovium samples. Synovium samples are collected from 2 patient populations: (1) patients with rheumatoid arthritis (RA) that are incomplete responders (IR) to disease-modifying antirheumatic drug (DMARD) treatment (DMARD-IR), and (2) patients with rheumatoid arthritis (RA) that are incomplete responders (IR) to TNF Inhibitor treatment (TNF-IR).

Full transcriptomic RNA sequencing is carried out on synovium samples collected from 2 patient populations as compared to healthy patients (control).

In brief, synovium samples are collected. After removal of ribosomal RNA and globin transcripts with the Ribo-Zero Globin Removal kit (Illumina), stranded libraries are prepared with the TruSeq Library prep kit (Illumina) and hybridized to a flow cell for sequencing with the Illumina HiSeq platform. Raw RNAseq output counts are log 2 normalized using the R DESeq2 package. The top 5,000 row variance (top5k rowVar) genes determined using standard deviation between samples are retained for further analysis.

The top 5,000 row variance genes are analyzed by a suite of gene expression technologies, including Multiscale Embedded Gene Co-expression Network Analysis (MEGENA) to generate gene coexpression modules which are functionally annotated and correlated to various demographic traits, clinical features, and laboratory assays.

In brief, the MEGENA R package is used to generate a gene coexpression network by inputting the top5k rowVar genes. MEGENA multi-scale clustering analysis (MCA) formed lineages of gene modules followed by identification of densely intraconnected hub genes using multi-scale hub analysis (MHA). Modules are assigned “lineage” names based on their multiscale pedigree from the root MEGENA module. The prcomp package is utilized to perform singular value decomposition and calculate MEGENA module eigengenes (MEs), equivalent to the first principal component calculated amongst the variance of a given MEGENA module. MEGENA MEs are correlated to the numerically encoded sample traits.

A heatmap is generated using ComplexHeatmap visualizing the top sample trait correlations to the MEGENA modules that are significantly correlated to cohort (or cluster). Module gene symbols are used to programmatically query the STRING database and calculate the percentage of genes within a given module predicted to have known protein-protein interactions (PPI) ranging from 0 to 100%.

A gene set variation analysis (GSVA) (GSVA (V1.25.0) R software package) is carried out as a non-parametric, unsupervised method for estimating the variation of pre-defined gene sets over all MEGENA module log 2 gene expression values. Input genes are employed only if the interquartile range (IQR) of their expression across the samples is greater than 0. Enrichment scores (GSVA scores) are calculated non-parametrically using a Kolmogorov Smirnoff (KS)-like random walk statistic. The enrichment scores(ES) are the largest positive and negative random walk deviations from zero, respectively, for a particular sample amongst the module gene set. The GSVA scores are used as input for unsupervised stable k-means clustering, and different disease phenotypes or clusters are identified. GSVA is performed using the significant MEGENA modules as gene signatures.

The MEs of the significant MEGENA modules are correlated to mean gene expression of a given module per patient and visualized using Complex heatmap. The heatmap's columns show different disease phenotypes or clusters are identified after patients are clustered using idealized k-means clustering. The heatmap's rows show various immune cell type and process modules (e.g., modules associated to inflammation, modules associated to cells, modules associated to JAK/TYK2, modules associated to steroids) used to identify the different disease phenotypes/clusters/patient subsets. In brief, the modules, including those for steroid response and JAK/TYK pathways (e.g., steroid gene markers, JAK1, JAK2, and TYK2) can be associated to subsets with enriched treatment targets. Overall, stable k-means clustering of gene coexpression modules effectively segregates patient subsets into different disease phenotypes which may be associated to different treatment targets and/or responsive to different treatments.

### Example 9: Molecular Endotypes of RA Patient Synovium Samples Before and After Treatments

A method is carried out to characterize the molecular landscape of patients with rheumatoid arthritis (RA) by analyzing gene expression profiles from synovium samples. Synovium samples are collected from 3 patient populations: (1) patients with rheumatoid arthritis (RA); (2) patients with rheumatoid arthritis (RA) that are incomplete responders (IR) to disease-modifying antirheumatic drug (DMARD) treatment (DMARD-IR); (3) RA DMARD-IR patients that are treated with TNFi.

Full transcriptomic RNA sequencing is carried out on synovium samples collected from 3 patient populations as compared to healthy patients (control).

In brief, synovium samples are collected. After removal of ribosomal RNA and globin transcripts with the Ribo-Zero Globin Removal kit (Illumina), stranded libraries are prepared with the TruSeq Library prep kit (Illumina) and hybridized to a flow cell for sequencing with the Illumina HiSeq platform. Raw RNAseq output counts are log 2 normalized using the R DESeq2 package. The top 5,000 row variance (top5k rowVar) genes determined using standard deviation between samples are retained for further analysis.

The top 5,000 row variance genes are analyzed by a suite of gene expression technologies, including Multiscale Embedded Gene Co-expression Network Analysis (MEGENA) to generate gene coexpression modules which are functionally annotated and correlated to various demographic traits, clinical features, and laboratory assays.

In brief, the MEGENA R package is used to generate a gene coexpression network by inputting the top5k rowVar genes. MEGENA multi-scale clustering analysis (MCA) formed lineages of gene modules followed by identification of densely intraconnected hub genes using multi-scale hub analysis (MHA). Modules are assigned “lineage” names based on their multiscale pedigree from the root MEGENA module. The prcomp package is utilized to perform singular value decomposition and calculate MEGENA module eigengenes (MEs), equivalent to the first principal component calculated amongst the variance of a given MEGENA module. MEGENA MEs are correlated to the numerically encoded sample traits.

A heatmap is generated using ComplexHeatmap visualizing the top sample trait correlations to the MEGENA modules that are significantly correlated to cohort (or cluster.) Module gene symbols are used to programmatically query the STRING database and calculate the percentage of genes within a given module predicted to have known protein-protein interactions (PPI) ranging from 0 to 100%.

A gene set variation analysis (GSVA) (GSVA (V1.25.0) R software package) is carried out as a non-parametric, unsupervised method for estimating the variation of pre-defined gene sets over all MEGENA module log 2 gene expression values. Input genes are employed only if the interquartile range (IQR) of their expression across the samples was greater than 0. Enrichment scores (GSVA scores) are calculated non-parametrically using a Kolmogorov Smirnoff (KS)-like random walk statistic. The enrichment scores(ES) are the largest positive and negative random walk deviations from zero, respectively, for a particular sample amongst the module gene set. The GSVA scores are used as input for unsupervised stable k-means clustering, and different disease phenotypes or clusters are identified. GSVA is performed using the significant MEGENA modules as gene signatures.

The MEs of the significant MEGENA modules are correlated to mean gene expression of a given module per patient and visualized using Complex heatmap. The heatmap's columns show different disease phenotypes or clusters are identified after patients are clustered using idealized k-means clustering. The heatmap's rows show various immune cell type and process modules (e.g., modules associated to inflammation, modules associated to cells, modules associated to JAK/TYK2, modules associated to steroids, modules associated to GC steroids, LUGENE modules, MEGENA modules) used to identify the different disease phenotypes/clusters/patient subsets. The heatmap color intensity visualizes the enrichment of gene signature for each patient as compared to one or more modules. In brief, the various immune cell type and process modules can be associated to subsets with enriched treatment targets. Overall, stable k-means clustering of gene coexpression modules effectively segregates patient subsets into different disease phenotypes which may be associated to different treatment targets and/or responsive to different treatments.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-120. (canceled)

121. A method for classifying an endotype of a subject with rheumatoid arthritis, the method comprising:
(a) analyzing a subject data set comprising or derived from gene expression data of at least 2 genes or orthologs thereof from a biological sample obtained or derived from the subject, wherein the at least 2 genes are selected from at least 2 different gene sets, and wherein the at least 2 different gene sets are selected from:
TABLE 2,
TABLE 3,
TABLE 4, or
TABLE 5; and
(b) classifying the subject as having the endotype of rheumatoid arthritis, using a machine-learning model configured to process gene expression levels of at least a subset of the at least 2 gene sets in the gene expression data.

122. The method of claim 121, further comprising (c) administering a treatment to the subject based on the subject being classified as having the endotype of rheumatoid arthritis, wherein the treatment comprises a immunoregulator, a immunosuppressant, a steroid, an anti-inflammatory, a JAK inhibitor, a TNF inhibitor, a baricitinib, a corticosteroid, a nonsteroidal anti-inflammatory drug (NSAID), a tofacitinib, a upadacitinib, a deucravacitinib, a brepocitinib, a disease-modifying anti-rheumatic drug (DMARD), a conventional synthetic DMARD (csDMARD), a targeted synthetic DMARD (tsDMARD), a biologic DMARD (bDMARD), a biologic treatment, a TYK2 inhibitor, a TYK2/JAK inhibitor, a combination inhibitor, a monoclonal antibody, an anti-TNF biologic, an anti-IL-6 biologic, an anti-IL-17 biologic, an anti-IL-12/23 biologic, an anti-CD28 biologic, or a combination thereof.

123. The method of claim 122, wherein the administering comprises oral administration of the treatment to the subject.

124. The method of claim 121, wherein the at least 2 genes comprise a plurality of genes from each gene set of the at least 2 different gene sets.

125. The method of claim 123, wherein the at least 2 genes comprise each gene from the at least 2 different gene sets.

126. The method of claim 121, wherein the at least 2 different gene sets are selected from Table 2.

127. The method of claim 121, wherein the at least 2 different gene sets are selected from Table 3.

128. The method of claim 121, wherein the at least 2 different gene sets are selected from Table 4.

129. The method of claim 121, wherein the at least 2 different gene sets are selected from Table 5.

130. The method of claim 121, wherein the at least 2 genes are selected from at least 20 different gene sets.

131. The method of claim 121, wherein the at least 2 genes are selected from at least 50 different gene sets.

132. The method of claim 121, wherein the at least 2 genes comprise at least 20 genes.

133. The method of claim 121, wherein the at least 2 genes comprise at least 50 genes.

134. The method of claim 121, further comprising, prior to (a), obtaining the biological sample from the subject.

135. The method of claim 134, wherein the biological sample comprises a blood sample.

136. The method of claim 134, wherein the biological sample comprises a synovial sample.

137. The method of claim 134, further comprising performing ribonucleic acid sequencing (RNA-seq) analysis on the biological sample to generate the subject data set.

138. The method of claim 134, further comprising performing quantitative polymerase chain reaction (qPCR) on the biological sample to generate the subject data set.

139. The method of claim 121, further comprising generating an electronic report indicative of the endotype of the subject.

140. The method of claim 139, wherein the electronic report further comprises a treatment recommendation for the subject, determined based at least in part of the endotype of the subject.
